(19) United States
(12) Patent Application Publication
(10) Pub. No.: US 2003/0073824 A1
(43) Pub. Date: Apr. 17, 2003

(54) DNA encoding methymycin and pikromycin

(76) Inventors: David H. Sherman, St. Louis Park, MN (US); Hung-Wen Liu, Austin, TX (US); Yongquan Xue, St. Paul, MN (US); Lishan Zhao, Carlsbad, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/988,384

(22) Filed: Nov. 19, 2001

Related U.S. Application Data
(63) Continuation of application No. PCT/US99/14398, filed on Jun. 25, 1999.

(51) Int. Cl. C07H 17/08; C07H 21/04; C12P 19/60; C12P 21/02; C12N 1/21; C12N 15/74

(52) U.S. Cl. 536/7.1; 536/23.2; 435/252.3; 435/320.1; 435/69.1; 435/75

(57) ABSTRACT

A biosynthetic gene cluster for methymycin and pikromycin as well as a biosynthetic gene cluster for desosamine is provided.

### STATEMENT OF GOVERNMENT RIGHTS

This invention was made with a grant from the Government of the United States of America (grants GM48562, GM35906 and GM54346 from the National Institutes of Health and a grant from the Office of Naval Research). The Government may have certain rights in the invention.

### BACKGROUND OF THE INVENTION

Polyhydroxyalkanoates (PHAs) are one class of biodegradable polymers. The first identified member of the PHAs thermoplastics was polyhydroxybutyrate (PHB), the polymeric ester of D(−)-3-hydroxybutyrate. The biosynthetic pathway of PHB in the gram negative bacterium Alcaligenes eutrophus is depicted in FIG. 1. PHAs related to PHB differ in the structure of the pendant arm, R (FIG. 2). For example, R═CH₃ in PHB, while R═CH₂CH₃ in polyhydroxyvalerate, and R═(CH₂)₄CH₃ in polyhydroxyoctanoate.

The genes responsible for PHB synthesis in A. eutrophus have been cloned and sequenced. (Peoples et al., J. Biol. Chem., 264, 15293 (1989); Peoples et al., J. Biol. Chem., 264, 15298 (1989)). Three enzymes: β-ketothiolase (phbA), acetoacetyl-CoA reductase (phbB), and PHB synthase (phbC) are involved in the conversion of acetyl-CoA to PHB. The PHB synthase gene encodes a protein of Mᵣ=63,900 which is active when introduced into E. coli (Peoples et al., J. Biol. Chem., 264, 15298 (1989)).

Although PHB represents the archetypical form of a biodegradable thermoplastic, its physical properties preclude significant use of the homopolymer form. Pure PHB is highly crystalline and, thus, very brittle. However, unique physical properties resulting form the structural characteristics of the R groups in a PHA copolymer may result in a polymer with more desirable characteristics. These characteristics include altered crystallinity, UV weathering resistance, glass to rubber transition temperature (T_{g}), melting temperature of the crystalline phase, rigidity and durability (Holmes et al., EPO 00052 459; Anderson et al., Microbiol. Rev., 54, 450 (1990)). Thus, these polyesters behave as thermoplastics, with melting temperatures of 50-180° C., which can be processed by conventional extension and molding equipment.

Traditional strategies for producing random PHA copolymers involve feeding short- and long-chain fatty acid monomers to bacterial cultures. However, this technology is limited by the monomer units which can be incorporated into a polymer by the endogenous PHA synthase and the expense of manufacturing PHAs by existing fermentation methods (Haywood et al., FEMS Microbiol. Lett., 57, 1 (1989); Poi et al., Int. J. Biol. Macrornol., 12, 106 (1990); Steinbuchel et al., In: Novel Biomaterials from Biological Sources. D. Byron (ed.), MacMillan, N.Y. (1991); Valentin et al., Appl. Microbiol. Biotechnical, 36, 507 (1992)).

The production of diverse hydroxyacylCoA monomers for homo- and co-polymeric PHAs also occurs in some bacteria through the reduction and condensation pathway of fatty acids. This pathway employs a fatty acid synthase (FAS) which condenses malonate and acetate. The resulting β-keto group undergoes three processing steps, β-keto reduction, dehydration, and enoyl reduction, to yield a fully saturated butyryl unit. However, this pathway provides only a limited array of PHA monomers which vary in alkyl chain length but not in the degree of alkyl group branching, saturation, or functionalization along the acyl chain.

The biosynthesis of polyketides, such as erythromycin, is mechanistically related to formation of long-chain fatty acids. However, polyketides, in contrast to FASs, retain ketone, hydroxyl, or olefinic functions and contain methyl or ethyl side groups interspersed along an acyl chain comparable in length to that of common fatty acids. This asymmetry in structure implies that the polyketide synthase (PKS), the enzyme system responsible for formation of these molecules, although mechanistically related to a FAS, results in an end product that is structurally very different than that of a long-chain fatty acid.

Because PHAs are biodegradable polymers that have the versatility to replace petrochemical-based thermoplastics, it is desirable that new, more economical methods be provided for the production of defined PHAs. Thus, what is needed are methods to produce recombinant PHA monomer synthases for the generation of PHA polymers.

Moreover, there is a continuing need for the identification and isolation of novel polyketide synthase genes, e.g., a polyketide synthase which encodes polypeptides that synthesize an antibiotic such as a macrolide.

### SUMMARY OF THE INVENTION

The invention provides an isolated and purified nucleic acid segment comprising a nucleic acid sequence comprising a sugar (desosamine) biosynthetic gene cluster, a biologically active variant or fragment thereof, wherein the nucleic acid sequence is not derived from the eryC gene cluster of Saccharopolyspora erythraea. As described hereinbelow, the desosamine biosynthetic gene cluster from Streptomyces venezuelae was isolated, cloned and sequenced. The isolated nucleic acid segment comprising the gene cluster preferably includes a nucleic acid sequence comprising SEQ ID NO:3, or a fragment or variant thereof. The cluster was found to encode nine polypeptides including DesI (e.g., SEQ ID NO:8 encoded by SEQ ID NO:7), DesII (e.g., SEQ ID NO:10 encoded by SEQ ID NO:9), DesIII (e.g., SEQ ID NO:12 encoded by SEQ ID NO:11), DesIV (e.g., SEQ ID NO:14 encoded by SEQ ID NO:13), DesV (e.g., SEQ ID NO:16 encoded by SEQ ID NO:15), DesVI (e.g., SEQ ID NO: 18 encoded by SEQ ID NO: 17), DesVII (e.g., SEQ ID NO:20 encoded by SEQ ID NO:19), DesVIII (e.g., SEQ ID NO:22 encoded by SEQ ID NO:21), and DesR (e.g., SEQ ID NO:24 encoded by SEQ ID NO:23) (see FIG. 24). It is also preferred that the nucleic acid segment of the invention encoding DesR is not derived from the eryB gene cluster of Saccharopolyspora erythraea or the oleD gene from Streptomyces antibioticus. Preferably, the nucleic acid segment comprising the desosamine biosynthetic gene cluster hybridizes under moderate, or more preferably stringent, hybridization conditions to SEQ ID NO:3, or a fragment thereof. Moderate and stringent hybridization conditions are well known to the art, see, for example sections 9.47-9.51 of Sambrook et al. (Molecular Cloning: A Laboratory Manual, Cold Spring Harbor Laboratory, Cold Spring Harbor, N.Y. (1989). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS.

The invention also provides a variant polypeptide having at least about 80%, more preferably at least about 90%, and even more preferably at least about 95%, but less than 100%, contiguous amino acid sequence identity to the polypeptide having an amino acid sequence comprising SEQ ID NO:8, SEQ ID NO:10, SEQ ID NO:12, SEQ ID NO:14, SEQ ID NO: 16, SEQ ID NO: 18, SEQ ID NO:20, SEQ ID NO:22, SEQ ID NO:24, or a fragment thereof A preferred variant polypeptide, or a subunit or fragment of a polypeptide, of the invention includes a variant or subunit polypeptide having at least about 1%, more preferably at least about 10%, and even more preferably at least about 50%, the activity of the polypeptide having the amino acid sequence comprising SEQ ID NO:8, SEQ ID NO:10, SEQ ID NO:12, SEQ ID NO:14, SEQ ID NO:16, SEQ ID NO:18, SEQ ID NO:20, SEQ ID NO:22, or SEQ ID NO:24. Thus, for example, the glycosyltransferase activity of a polypeptide of SEQ ID NO:20 can be compared to a variant of SEQ ID NO:20 having at least one amino acid substitution, insertion, or deletion relative to SEQ ID NO:20.

A variant nucleic acid sequence of the invention has at least about 80%, more preferably at least about 90%, and even more preferably at least about 95%, but less than 100%, contiguous nucleic acid sequence identity to a nucleic acid sequence comprising SEQ ID NO:3, SEQ D NO:7, SEQ ED NO:9, SEQ ID NO:11, SEQ ID NO:13, SEQ ID NO:15, SEQ ID NO:17, SEQ ID NO:19, SEQ ID NO:21, SEQ ID NO:23, or a fragment thereof.

Also provided is an expression cassette comprising a nucleic acid sequence comprising a desosamine biosynthetic gene cluster, a biologically active variant or fragment thereof operably linked to a promoter functional in a host cell, as well as host cells comprising an expression cassette of the invention. Thus, the expression cassettes of the invention are useful to express individual genes within the cluster, e.g., the desR gene which encodes a glycosidase or the des VII gene which encodes a glycosyltransferase having relaxed substrate specificity for polyketides and deoxysugars, i.e., the glycosyltransferase processes sugar substrates other than TDP-desosamine. Thus, the desVII gene can be employed in combinatorial biology approaches to synthesize a library of macrolide compounds having various polyketide and deoxysugar structures. Moreover, the expression of a glycosylase in a host cell which synthesizes a macrolide antibiotic may be useful in a method to reduce toxicity of, e.g., inactivate, the antibiotic. For example, a host cell which produces the antibiotic is transformed with an expression cassette encoding the glycosyltransferase. The recombinant glycosyltransferase is expressed in an amount that reversibly inactivates the antibiotic. To activate the antibiotic, the antibiotic, preferably the isolated antibiotic which is recovered from the host cell, is contacted with an appropriate native or recombinant glycosidase.

Preferably, the nucleic acid segment encoding desosamine in the expression cassette of the invention is not derived form the eryC gene cluster of Saccharopolyspora erythraea. Preferred host cells are prokaryotic cells, although eukaryotic host cells are also envisioned. These host cells are useful to express desosamine, analogs or derivatives thereof as well as individual polypeptides which can then be isolated from the host cell. Also provided is an expression cassette or host cell comprising antisense sequences from at least a portion of the desosamine biosynthetic gene cluster.

Another embodiment of the invention is a recombinant host cell, e.g., a bacterial cell, in which at least a portion of a nucleic acid sequence encoding desosamine in the host chromosome is disrupted, e.g., deleted or interrupted (e.g., by an insertion) with heterologous sequences, or substituted with a variant nucleic acid sequence of the invention, so as to alter, preferably so as to result in a decrease or lack of, desosamine synthesis and/or so as to result in the synthesis of an analog or derivative of desosamine. Preferably, the nucleic acid sequence which is disrupted is not derived from the eryC gene cluster of Saccharopolyspora erythraea. Thus, the recombinant host cell of the invention has at least one gene, i.e., desI, desII, desIII, desIV, desV desVI, desVII, desVIII or desR, which is disrupted. One embodiment of the invention includes a recombinant host cell in which the desVI gene, which encodes an N-methyltransferase, is disrupted, for example, by replacement with an antibiotic resistance gene. Preferably, such a host cell produces an aglycone having an N-acetylated aminodeoxy sugar, 10-deoxy-methylonide, a compound of formula (7), a compound of formula (8), or a combination thereof. Thus, the deletion or disruption of the desVI gene may be useful in a method for preparing novel sugars.

Another preferred embodiment of the invention is a recombinant bacterial host cell in which the desR gene, which encodes a glycosidase such as β-glucosidase, is disrupted. Preferably, the host cell synthesizes C-2′β-glucosylated macrolide antibiotics, for example, a compound of formula (13), a compound of formula (14), or a combination thereof. Therefore, the invention further provides a compound of formula (8), (9), (13) or (14). It will be appreciated by those skilled in the art that each atom of the compounds of the invention having a chiral center may exist in and be isolated in optically active and racemic forms. Some compounds may exhibit polymorphism. It is to be understood that the present invention encompasses any racemic, optically active, polymorphic or stereoisomeric form, or mixtures thereof, of a compound of the invention, which possess the useful properties described herein, it being well known in the art how to prepare optically active forms (for example, by resolution of the racemic form by recrystallization techniques, by synthesis from optically active starting materials, by chiral synthesis, or by chromatographic separation using a chiral stationary phase) and how to determine activity using the standard tests described herein, or using other similar tests which are well known in the art.

Also provided is a method for directing the biosynthesis of specific glycosylation-modified polyketides by genetic manipulation of a polyketide-producing microorganism. The method comprises introducing into a polyketide-producing microorganism a DNA sequence encoding enzymes in desosamine biosynthesis, e.g., a DNA sequence comprising SEQ ID NO:3, a variant or fragment thereof, so as to yield a microorganism that produces specific glycosylation-modified polyketides. Alternatively, an anti-sense DNA sequence of the invention may be employed. Then the glycosylation-modified polyketides are isolated from the microorganism. It is preferred that the DNA sequence is modified so as to result in the inactivation of at least one enzymatic activity in sugar biosynthesis or in the attachment of the sugar to a polyketide.

Further provided is an isolated and purified nucleic acid segment comprising a nucleic acid sequence comprising a macrolide biosynthetic gene cluster (the “met/pik” or “pik” gene cluster) encoding polypeptides that synthesize methymycin, pikromycin, neomethymycin, narbomycin, or a combination thereof, or a biologically active variant or fragment thereof. It is preferred that the nucleic acid segment comprises SEQ ID NO:5, or a fragment or variant thereof, or hybridizes under moderate or more preferably stringent, conditions to SEQ ID NO:5 or a fragment thereof. It is also preferred that the isolated and purified nucleic acid segment is from Streptomyces sp., such as Streptomyces venezuelae (e.g., ATCC 15439, ATCC 15068, MCRL 0306, SC 2366 or 3629), Streptomyces narbonensis (e.g., ATCC 19790), Streptomyces eurocidicus, Streptomyces zaomyceticus (MCRL 0405), Streptomyces flavochromogens, Streptomyces sp. AM400, and Streptomyces felleus, although isolated and purified nucleic acid from other organisms which produce methymycin, narbomycin, neomethymycin and/or pikromycin are also within the scope of the invention. The cloned genes can be introduced into an expression system and genetically manipulated so as to yield novel macrolide antibiotics, e.g., ketolides, as well as monomers for polyhydroxyalkanoate (PHA) biopolymers. Preferably, the nucleic acid sequence encodes PikR1 (e.g., SEQ ID NO:27 encoded by SEQ ID NO:26), PikR2 (e.g., SEQ ID NO:29 encoded by SEQ ID NO:28), PikAI (e.g., SEQ ID NO:31 encoded by SEQ ID NO:30), PikAII (e.g., SEQ ID NO:33 encoded by SEQ ID NO:32), PikARII (e.g., SEQ ID NO:35 encoded by SEQ ID NO:34), PikAIV (e.g., SEQ ID NO:37 encoded by SEQ ID NO:36), PikB (which is the desosamine gene cluster described above), PikC (e.g., SEQ ID NO:39 encoded by SEQ ID NO:38), and PikD (e.g., SEQ ID NO:41 encoded by SEQ ID NO:40), a variant or a fragment thereof, or hybridizes under moderate or preferably stringent conditions to such a nucleic acid sequence.

The invention also provides a variant polypeptide having at least about 80%, more preferably at least about 90%, and even more preferably at least about 95%, but less than 100%, contiguous amino acid sequence identity to the polypeptide having an amino acid sequence comprising SEQ ID NO:27, SEQ ID NO:29, SEQ ID NO:31, SEQ ID NO:33, SEQ ID NO:35, SEQ ID NO:37, SEQ ID NO:39, SEQ ID NO:41, or a fragment thereof. A preferred variant polypeptide, or a subunit or fragment of a polypeptide, of the invention includes a variant or subunit polypeptide having at least about 1%, more preferably at least about 10%, and even more preferably at least about 50%, the activity of the polypeptide having the amino acid sequence comprising SEQ ID NO:27, SEQ ID NO:29, SEQ ID NO:31, SEQ ID NO:33, SEQ ID NO:35, SEQ ID NO:37, SEQ ID NO:39, or SEQ ID NO:41. The activities of polypeptides of the macrolide biosynthetic pathway of the invention are described below.

A variant nucleic acid sequence of the pik biosynthetic gene cluster of the invention has at least about 80%, more preferably at least about 90%, and even more preferably at least about 95%, but less than 100%, contiguous nucleic acid sequence identity to a nucleic acid sequence comprising SEQ ID NO:5, SEQ ID NO:26, SEQ ID NO:28, SEQ ID NO:30, SEQ ID NO:32, SEQ ID NO:34, SEQ ID NO:36, SEQ ID NO:38, SEQ ID NO:40, or a fragment thereof.

The pikA gene encodes a polyketide synthase which synthesizes macrolactone 10-deoxymethonolide and narbolide, pikB encodes desosamine synthases which catalyze the formation and transfer of a deoxysugar moiety onto aglycones, the pikC gene encodes a P450 hydoxylase which catalyzes the conversion of YC-17 and narbomycin into methymycin, neomethymycin, and pikromycin, and the pikR 1, pikR2 (possibly one for a 12-membered ring and the other for a 14-membered ring) and desR genes which encode enzymes associated with bacterial self-protection. Thus, the isolated nucleic acid molecule of the invention encodes four active macrolide antibiotics two of which have a 12-membered ring while the other two have a 14-membered ring. The genetic mechanism underlying the alternative tennination of polyketide synthesis may be useful to prepare novel compounds, e.g., antibiotics, and PHA monomers. The invention further provides isolated and purified nucleic acid segments, e.g., in the form of an expression cassette, for each of the individual genes in the macrolide biosynthetic gene cluster. For example, the invention provides an isolated and purified pikAV gene that encodes a thioesterase II. In particular, the thioesterase may be useful to enhance the structural diversity of antibiotics and in PHA production, as the thioesterase modulates chain release and cyclization. For example, a thioesterase II gene having acyl-ACP coenzyme A transferase activity (e.g., a mutant pik TEII, bacterial, fungal or plant medium-chain-length thioesterase, an animal fatty acid thioesterase or a thioesterase from a polyketide synthase) is introduced at the end of a recombinant monomer synthase (see FIG. 36), which, in the presence of a PHA synthase, e.g., phaC1, produces a novel polyhydroxyalkanoate polymer. Alternatively, in the absence of a TEII domain, a fusion of a portion of PKS gene cluster with a PHA synthase may result in the transfer of an acyl chain from the PHA to the polymerase.

Also provided is a pikC gene that encodes a hydroxylase which is active at two positions on a 12-membered ring or at one position on a 14-membered ring. Such a gene may be particularly useful to prepare novel compounds through bioconversion or biotransformation.

The invention also provides an expression cassette comprising a nucleic acid segment comprising a macrolide biosynthetic gene cluster encoding polypeptides that synthesize methymycin, pikromycin, neomethymycin, narbomycin, or a combination thereof, or a biologically active variant or fragment thereof, operably linked to a promoter functional in a host cell. Further provided is a host cell comprising the nucleic acid segment encoding methymycin, pikromycin, neomethymycin, narbomycin, or a combination thereof, or a biologically active variant or fragment thereof Moreover, the invention provides isolated and purified polypeptides of the invention, preferably obtained from host cells having the nucleic acid molecules of the invention. In addition, expression cassettes and host cells comprising antisense sequences of at least a portion of the macrolide biosynthetic gene cluster of the invention are envisioned.

Yet another embodiment of the invention is a recombinant host cell, e.g., a bacterial cell, in which a portion of the macrolide biosynthetic gene cluster of the invention is disrupted or replaced with a heterologous sequence or a variant nucleic acid segment of the invention, so as to alter, preferably so as to result in a decrease or lack of methymycin, pikromycin, neomethymycin, narbomycin, or a combination thereof, and/or so as to result in the synthesis of novel macrolides. Therefore, the invention provides a recombinant host cell in which a pikAI gene, a pikAlI gene, a pikAIlI gene (12-membered rings), apikIV gene (14-membered rings), a pikB gene cluster, a pikAV gene, a pikC gene, a pikD gene, a pikR1 gene, a pikR2 gene, or a combination thereof, is disrupted or replaced. A preferred embodiment of the invention is a host cell wherein the pikB (e.g., the desVI and desV genes), pikA1, pikAV or pikC gene, is disrupted.

Although the sixth (final) condensation cycle is not required for 10-deoxymethynolide formation, as described hereinbelow genetic disruption of Pik module 6 (encoded by pikAIV) prevented production of both the 12- as well as the 14-membered ring macrolactones. Thus, expression of alternative forms of PikAIV controls the final step in polyketide chain elongation and termination. Specifically, an N-termninal truncated form of PikAIV leads to 10-deoxymethynolide formation while full-length PikAIV results in narbonolide production. The expression of a truncated PKS module represents a novel method of polyketide chain length determination. Moreover, as the expression of such a module may produce multiple polyketides, the use of such a module may result in the more rapid identification of novel products.

The invention also provides a method for combinatorial biosynthesis. The method comprises expressing in a host cell an expression cassette comprising a DNA fragment of a biosynthetic gene cluster, e.g., a polyketide synthase gene wherein the expression cassette is present on a plasmid, wherein the genome of the host cell comprises a portion of the gene which is different than the portion of the gene present on the plasmid. Preferably, the DNA fragment and the portion of the gene which is one the host chromosome together comprise the entire gene. Synchronized expression of genes from the plasmid and the chromosome thus creates a combinatorial pathway that produces a product. The smaller size of the plasmid facilitates gene manipulation so that a large library of recombinant pathways can thus be generated in a short time. Preferably, the DNA fragment and the portion of the gene cluster on the host chromosome are linked to the native promoter, e.g., pik genes are linked to PpikA.

Moreover, as the nucleic acid segment comprising the macrolide biosynthetic gene cluster of the invention encodes a polyketide synthase, modules of that synthase are useful in methods to prepare recombinant polyhydroxyalkanoate monomer synthases and polymers in addition to macrolide antibiotics and derivatives thereof.

Thus, the invention provides an isolated and purified DNA molecule comprising a first DNA segment encoding a first module and a second DNA segment encoding a second module, wherein the DNA segments together encode a recombinant polyhydroxyalkanoate monomer synthase, and wherein at least one DNA segment is derived from the pikA gene cluster of Streptomyces venezuelae. Preferably, no more than one DNA segment is derived from the eryA gene cluster of Saccharopolyspora erythraea. In one embodiment of the invention, the 3′ most DNA segment of the isolated DNA molecule of the invention encodes a thioesterase II. Also provided is an expression cassette comprising a nucleic acid molecule encoding the polyhydroxyalkanoate monomer synthase operably linked to a promoter fumctional in a host cell.

Yet another embodiment of the invention is a method of providing a polyhydroxyalkanoate monomer. The method comprises introducing into a host cell a DNA molecule comprising a DNA segment encoding a recombinant polyhydroxyalkanoate monomer synthase operably linked to a promoter functional in the host cell. The DNA molecule comprises a plurality of DNA segments, e.g., a first module and a second module, wherein at least one DNA segment is derived from the pikA gene cluster of Streptomyces venezuelae. The DNA encoding the recombinant polyhydroxyalkanoate monomer synthase is then expressed in the host cell so as to generate a polyhydroxyalkanoate monomer. Optionally, a second DNA molecule may be introduced into the host cell. The second DNA molecule comprises a DNA segment encoding a polyhydroxyalkanoate synthase operably linked to a promoter functional in the host cell. The two DNA molecules are expressed in the host cell so as to generate a polyhydroxyalkanoate polymer.

Another embodiment of the invention is an isolated and purified DNA molecule comprising a first DNA segment encoding a fatty acid synthase and a second DNA segment encoding a module from the pikA gene cluster of Streptomyces venezuelae. Such a DNA molecule can be employed in a method of providing a polyhydroxyalkanoate monomer. Thus, a DNA molecule comprising a first DNA segment encoding a fatty acid synthase and a second DNA segment encoding a polyketide synthase is introduced into a host cell. The first DNA segment is 5′ to the second DNA segment and the first DNA segment is operably linked to a promoter functional in the host cell. The first DNA segment is linked to the second DNA segment so that the linked DNA segments express a fusion protein. The DNA molecule is expressed in the host cell so as to generate a polyhydroxyalkanoate monomer.

Further provided is a method of providing a polyhydroxyalkanoate monomer synthase. The method comprises introducing an expression cassette comprising a DNA molecule encoding a polyhydroxyalkanoate synthase operably linked to a promoter functional in a host cell. The DNA molecule comprises a first DNA segment encoding a first module and a second DNA segment encoding a second module wherein the DNA segments together encode a polyhydroxyalkanoate monomer synthase. At least one DNA segment is derived from the pikA gene cluster of Streptomyces venezuelae. The DNA molecule is expressed in the host cell. Optionally, the DNA molecule further comprises a DNA segment encoding a polyhydroxyalkanoate synthase. Alternatively, a second, separate DNA molecule encoding a polyhydroxyalkanoate synthase is introduced into the host cell.

A further embodiment of the invention is an isolated and purified DNA molecule comprising a DNA segment which encodes a Streptomyces venezuelae polyketide synthase, e.g., a polyhydroxyalkanoate monomer synthase, a biologically active variant or subunit (fragment) thereof. Preferably, the DNA segment encodes a polypeptide having an amino acid sequence comprising SEQ ID NO:2. Preferably, the DNA segment comprises SEQ ID NO:1. The DNA molecules of the invention are double stranded or single stranded. A preferred embodiment of the invention is a DNA molecule that has at least about 70%, more preferably at least about 80%, and even more preferably at least about 90%, but less than 100%, contiguous sequence identity to the DNA segment comprising SEQ ID NO:1, e.g., a “variant” DNA molecule. A variant DNA molecule of the invention can be prepared by methods well known to the art, including oligonucleotide-mediated mutagenesis. See Adelman et al., DNA, 2, 183 (1983) and Sambrook et al., Molecular Cloning. A Laboratory Manual (1989).

The invention also provides an isolated, purified polyhydroxyalkanoate monomer synthase, e.g., a polypeptide having an amino acid sequence comprising SEQ ID NO:2, a biologically active subunit, or a biologically active variant thereof. Thus, the invention provides a variant polypeptide having at least about 80%, more preferably at least about 90%, and even more preferably at least about 95%, but less than 100%, contiguous amino acid sequence identity to the polypeptide having an amino acid sequence comprising SEQ ID NO:2. A preferred variant polypeptide, or a subunit of a polypeptide, of the invention includes a variant or subunit polypeptide having at least about 10%, more preferably at least about 50%, and even more preferably at least about 90%, the activity of the polypeptide having the amino acid sequence comprising SEQ ID NO:2. Preferably, a variant polypeptide of the invention has one or more conservative amino acid substitutions relative to the polypeptide having the amino acid sequence comprising SEQ ID NO:2. For example, conservative substitutions include aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. The biological activity of a polypeptide of the invention can be measured by methods well known to the art, including but not limited to, methods described hereinbelow.

Thus, the modules encoded by the nucleic acid segments of the invention may be employed in the methods described hereinabove to prepare polyhydroxyalkanoates of varied chain length or having various side chain substitutions and/or to prepare glycosylated biopolymers.

The compounds produced by the recombinant host cells of the invention are useful as biopolymers, e.g., in packaging or biomedical applications, to engineer PHA monomer synthases, or to prepare biologically active agents, such as those useful to prepare a medicament for the treatment of a pathological condition or a symptom in a mammal, e.g., a human. The agents include pharmaceuticals such as chemotherapeutic agents, immunosuppressants, agents to treat asthma, chronic obstructive pulmonary disease as well as other diseases involving respiratory inflammation, cholesterol-lowering agents, or macrolide-based antibiotics which are active against a variety of organisms, e.g., bacteria, including multi-drug-resistant pneumococci and other respiratory pathogens, as well as viral and parasitic pathogens; or as crop protection agents (e.g., fungicides or insecticides) via expression of polyketides in plants. Methods employing these compounds, e.g., to treat a mammal, bird or fish in need of such therapy, such as a patient having a bacterial, viral or parasitic infection, cancer, respiratory disease, or in need of immunosuppression, e.g., during cell, tissue or organ transplantation, are also envisioned.

### BRIEF DESCRIPTION OF THE FIGURES

FIG. 1. The PHB biosynthetic pathway in A. eutrophus.

FIG. 2. Molecular structure of common bacterial PHAs. Most of the known PHAs are polymers of 3-hydroxy acids possessing the general formula shown. For example, R═CH₃ in PHB, T═CH₂CH₃ in polyhydroxyvalerate (PHV), and R═(CH₂)₄CH₃ in polyhydroxyoctanoate (PHO).

FIG. 3. Comparison of the natural and recombinant pathways for PHB synthesis. The three enzymatic steps of PHB synthesis in bacteria involving 3-ketothiolase, acetoacetyl-CoA reductase, and PHB synthase are shown on the left. The two enzymatic steps involved in PHB synthesis in the pathway in Sf21 cells containing a rat fatty acid synthase with an inactivated dehydrase domain (ratFAS206) are shown on the right.

FIG. 4. Schematic diagram of the molecular organization of the tyl polyketide synthase (PKS) gene cluster. Open arrows correspond to individual open reading frames (ORFs) and numbers above an ORF denote a multifunctional module or synthase unit (SU). AT=acyltransferase; ACP=acyl carrier protein; KS=β-ketoacyl synthase; KR=ketoreductase; DH=dehydrase; ER=enoyl reductase; TE=thioesterase; MM=methylmalonylCoA; M=malonyl CoA; EM=ethylmalonyl CoA. Module 7 in tyl is also known as Module F.

FIG. 5. Schematic diagram of the molecular organization of the met PKS gene cluster.

FIG. 6. Strategy for producing a recombinant PHA monomer synthase by domain replacement.

FIG. 7. (A) 10% SDS-PAGE gel showing samples from various stages of the purification of PHA synthase; lane 1, molecular weight markers; lane 2, total protein of uninfected insect cells; lane 3, total protein or insect cells expressing a rat FAS (200 kDa; Joshi et al., Biochem. J., 296, 143 (1993)); lane 4, total protein of insect cells expressing PHA synthase; lane 5, soluble protein from sample in lane 4; lane 6, pooled hydroxylapatite (HA) fractions containing PHA synthase. (B) Western analysis of an identical gel using rabbit-α-PHA synthase antibody as probe. Bands designated with arrows are: a, intact PHB synthase with N-terminal alanine at residue 7 and serine at residue 10 (A7/S10); b, 44 kDa fragment of PHB synthase with N-terminal alanine at residue 181 and asparagine at residue 185 (A181/N185); c, PHB synthase fragment of approximately 30 kDa apparently blocked based on resistance to Edman degradation; d, 22 kDa fragment with N-terminal glycine at residue 187 (G 187). Band d apparently does not react with rabbit-α-PHB synthase antibody (B, lane 6). The band of similar size in B, lane 4 was not further identified.

FIG. 8. N-terrninal analysis of PHA synthase purified from insect cells. (a) The expected N-terrninal 25 amino acid sequence of A. eutrophus PHA synthase. (b&c) The two N-terminal sequences determined for the A. eutrophus PHA synthase produced in insect cells. The bolded sequences are the actual N-termini determined.

FIG. 9. Spectrophotometric scans of substrate, 3-hydroxybutyrate CoA (HBCoA) and product, CoA. The wavelength at which the direct spectrophotometric assays were carried out (232 nm) is denoted by the arrow; substrate, HBCoA (●) and product, CoA (◯).

FIG. 10. Velocity of the hydrolysis of HBCoA as a function of substrate concentration. Assays were carried out in 40 or 200 μl assay volumes with enzyme concentration remaining constant at 0.95 mg/ml (3.8 μg/40 μl assay). Velocities were calculated from the linear portions of the assay curves subsequent to the characteristic lag period. The substrate concentration at half-optimal velocity, the apparent Kₘ value, was estimated to be 2.5 mM from this data.

FIG. 11. Double reciprocal plot of velocity versus substrate concentration. The concave upward shape of this plot is similar to results obtained by Fukui et al. (Arch. Microbiol., 110, 149 (1976)) with granular PHA synthase from Z. ramigera.

FIG. 12. Velocity of the hydrolysis of HBCoA as a function of enzyme concentration. Assays were carried out in 40 μl assay volumes with the concentration HBCoA remaining constant at 8 μM.

FIG. 13. Specific activity of PHA synthase as a function of enzyme concentration.

FIG. 14. pH activity curve for soluble PHA synthase produced using the baculovirus system. Reactions were carried out in the presence of 200 mM Pᵢ. Buffers of pH<10 were prepared with potassium phosphate, while buffers of pH>10 were prepared with the appropriate proportion of Na₃PO₄.

FIG. 15. Assays of the hydrolysis of HBCoA with varying amounts of PHA synthase. Assays were carried out in 40 μl assay volumes with the concentration of HBCoA remaining constant at 8 μM. Initial A₂₃₂ values, originally between 0.62 and 0.77, were normalized to 0.70. Enzyme amounts used in these assays were, from the uppermost curve, 0.38, 0.76, 1.14, 1.52, 1.90, 2.28, 2.66, 3.02, 3.42, 7.6, and 15.2 μg, respectively.

FIG. 16. SDS/PAGE analysis of proteins synthesized at various time points during infection of Sf21 cells. Approximately 0.5 mg of total cellular protein from various samples was fractionated on a 10% polyacrylamide gel. Samples include: uninfected cells, lanes 1-4, days 0, 1, 2, 3, respectively; infection with BacPAK6::phbC alone, lanes 5-8, days, 0, 1, 2, 3, respectively, infection with baculoviral clone containing ratFAS206 alone, lanes 9-12, days 0, 1, 2, 3, respectively; and ratFAS206 and BacPAK6 infected cells, lanes 13-16, days 0, 1, 2, 3, respectively. A=mobility of FAS, B=mobility of PHA synthase. Molecular weight standard lanes are marked M.

FIG. 17. Gas chromatographic evidence for PHB accumulation in Sf21 cells. Gas chromatograms from various samples are superimposed. PHB standard (Sigma) is chromatogram #7 showing a propylhydroxybutyrate elution time of 10.043 minutes (s, arrow). The gas chromatograms of extracts of the uninfected (#1); singly infected with ratFAS206 (#2, day 3); and singly infected with PHA synthase (#3, day 3) are shown at the bottom of the figure. Gas chromatograms of extracts of dual-infected cells at day 1 (#4), 2 (#5), and 3 (#6) are also shown exhibiting a peak eluting at 10.096 minutes (x, arrow). The peak of dual-infected, day 3 extract (#6) was used for mass spectrometry (MS) analysis.

FIG. 18. Gas chromatography-mass spectrometry analysis of PHB. The characteristic fragmentation of propylhydroxybutyrate at m/z of 43, 60, 87, and 131 is shown. A) standard PHB from bacteria (Sigma), and B) peak X from ratFAS206 and BacPAK6: phbC baculovirus infected, day 3 (#6, FIG. 17) Sf21 cells expressing rat FAS dehydrase inactivated protein and PHA synthase.

FIG. 19. Map of the vep (Streptomyces venezuelae polyene encoding) gene cluster.

FIG. 20. Plasmid map of pDHS502.

FIG. 21. Plasmid map of pDHS505.

FIG. 22. Cloning protocol for pDHS505.

FIG. 23. Nucleotide sequence (SEQ ID NO:1) and corresponding amino acid sequence (SEQ ID NO:22) of vep ORFI.

FIG. 24. Schematic diagram of the desosamine biosynthetic pathway and the enzymatic activity associated with each of the desosamine biosynthetic polypeptides.

FIG. 25. Schematic of the conversion of the inactive (diglycosylated) form of methymycin and pikromycin to the active form of methymycin and pikromycin.

FIG. 26. Schematic diagram of the desosamine biosynthetic pathway.

FIG. 27. Pathway for the synthesis of a compound of formula 7 and 8 in desVI mutants of Streptomyces.

FIG. 28. Structure and biosynthesis of methymycin, pikromycin, and related compounds in Streptomyces venezuelae ATCC 15439. Methymycin: R₁═OH, R₂═H, neomethymycin: R₁═H, R₂ ═OH; pikromycin: R₃═OH, narbomycin: R₃ ═H. Polyketide synthase components PikAI, PikAII, PikAIII, PikAIV, and PikAV are represented by solid bars. Each circle represents an enzymatic domain in the Pik PKS system. KS: β-ketoacyl-ACP synthase, AT: acyltransferase, ACP: acyl carrier protein, KR: β-ketoacyl-ACP reductase, DH: β-hydroxyl-thioester dehydratase, ER: enoyl reductase, KS^{Q}: a KS-like domain, KR with a cross: nonfunctional KR, TE: thioesterase domain, and TEII: type II thioesterase. Des represents all eight enzymes for desosamine biosynthesis and transfer and PikC is the cytochrome P450 monooxygenase responsible for hydroxylation at R₁, R₂, and R₃ positions (Xu et al., 1998).

FIG. 29. Organization of the pik cluster in S. venezuelae. Each arrow represents an open reading frame (ORF). The direction of transcription and relative sizes of the ORFs deduced from nucleotide sequence are indicated. The cluster is composed of four genetic loci: pikA, pikB (des), pikC, and pikR. Cosmid clones are denoted as overlapping lines.

FIG. 30. Conversion of YC-17 and narbomycin by PikC P450 hydroxylase.

FIG. 31. Nucleotide sequence (SEQ ID NO:5) and inferred amino acid sequence (SEQ ID NO:6) of the pik gene cluster.

FIG. 32. Nucleotide sequence (SEQ ID NO:3) and inferred amino acid sequence (SEQ ID NO:4) of the desosamine gene cluster.

FIG. 33. S. venezuelae AX916 construct useful to prepare a polyketide having a shorter chain length compared to wild-type pikA. pik module 2 is fused to pik module 5, and module 3 and 4 are deleted, so as to encode a three module PKS which produces two macrolides, a triketide and a tetraketide.

FIG. 34. Recombinant PKS having a wild-type thioesterase II.

FIG. 35. pAX703 construct, an expression and complementation vector. The PikTEII gene can be replaced with an EcoRI-NsiI fragment. The phaC1 gene can be replaced with a PacI-DraI fragment.

FIG. 36. Strategy for C7 polymer production. mTEII is a mutant pikTEII, an acyl-ACP CoA transferase; phaC1 is a PHA polymerase 1 from P. olivarus which may have racemase activity. In a strain having these constructs, AX916, a PHA polymer is produced.

FIG. 37. Strategy for C5 polymer production. A PHA polymerase gene phaC1 is directly fused to pik module 2, so as to result in a fusion that transfers an acyl chain from the PKS protein directly to the polymerase by the prosthetic group on the ACP domain of the PKS.

FIG. 38. Codons for specified amino acids.

FIG. 39. Exemplary and preferred amino acid substitutions.

FIG. 40. Plasmid complementation of S. venezuelae AX912. The relevant genotype (on the chromosome and on the plasmid) is listed on the left side and the corresponding phenotype is listed on the right side. The pikA genes are indicated by open arrows with divided boxes indicating domains in the PKS. An internal alternative translation start site for PikAIV is indicated by an * above the KS₆ domain and a hexa-histidine was introduced into mutant AX912 chromosome position marked by a) to facilitate the detection of PikAIV expression. Antibiotic production was determined following complementation of mutant AX912 with the corresponding plasmids. Antibiotic production was normalized by using AX912 as 0% and full-length pikAIV complementation (pDHS707) as 100% standards.

FIG. 41. Mechanistic models for alternative termination by PikAIV. Proteins PikAIII and PikAIV are stacked one on top of the other according to their order in polyketide biosynthesis (PikAI and PikAII are not shown). A sphere represents an enzymatic domain in the PKSs with its diameter proportional to the size of the domain. Each PKS module/protein was first dimerized (each peptide chain is shown as either red or blue) and then twisted 180 degrees to form a half helix following the model for erythromycin PKS (Staunton et al., 1999). Two sets of independent active sites are thus formed along two grooves of the helix that lead to the production of two polyketides in each biosynthetic cycle. A) Wild type S. venezuelae under culture conditions for pikromycin production. B) Wild type S. venezuelae under culture conditions for methymycin production. C) S. venezuelae AX912 (pDHS704) under culture conditions for methyrycin production. D) S. venezuelae AX912 (pDHS704) under culture conditions for pikromycin production. E) S. venezuelae AX912 (pDHS708) under culture conditions for pikromycin production. F) S. venezuelae AX912 (pDHS708) under culture conditions for methymycin production. Gene products expressed from the plasmid construct used for complementation are underlined.

FIG. 42. Pathway for desosamine biosynthesis.

FIG. 43. Schematic of pathway leading to methymycin/neomethymycin analogs 18 and 19.

FIG. 44. Macrolide having D-quinovose.

FIG. 45. Products produced by desI mutant.

FIG. 46. Pik sequences from Streptomyces spp. A) PikA3-pikA4 from S. venezulae ATCC 15068 (SEQ ID NO:54). B) PikA3-pikA4 from S. narbonesis ATCC 19790 (SEQ ID NO:55). C) TEII gene from S. venezulae ATCC 15068 (SEQ ID NO:56). D) TEII gene from S. narbonesis ATCC 19790 (SEQ ID NO:57).

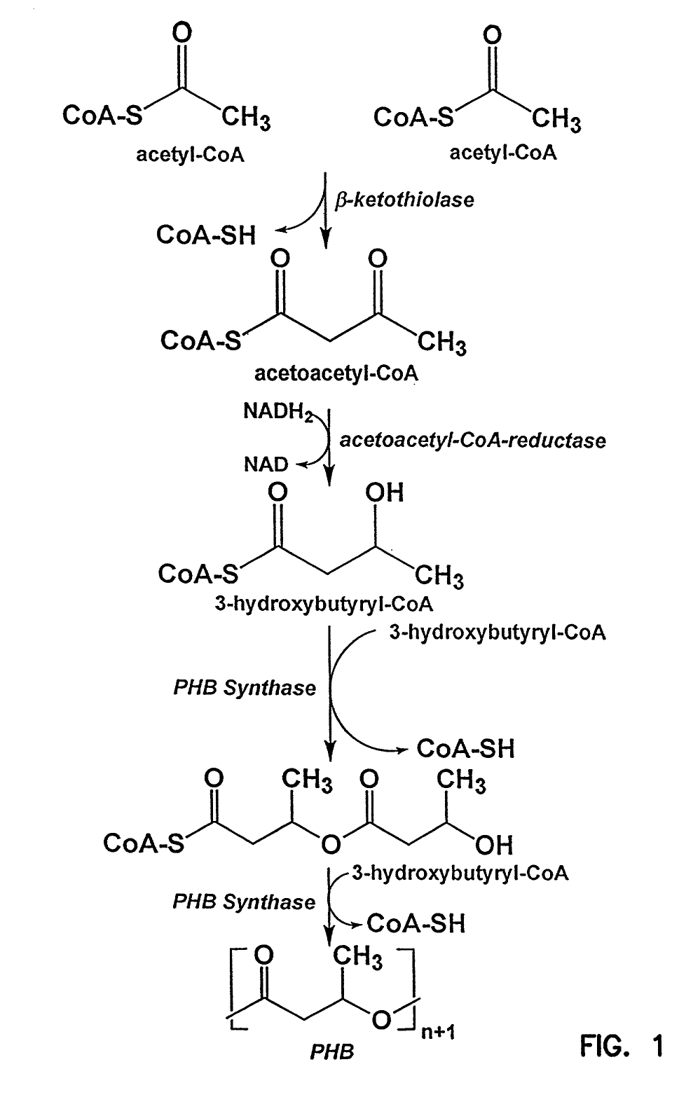

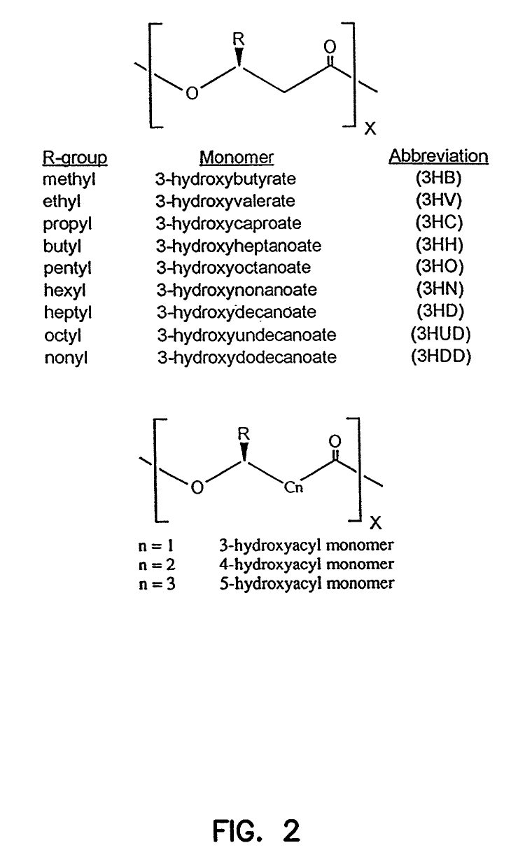

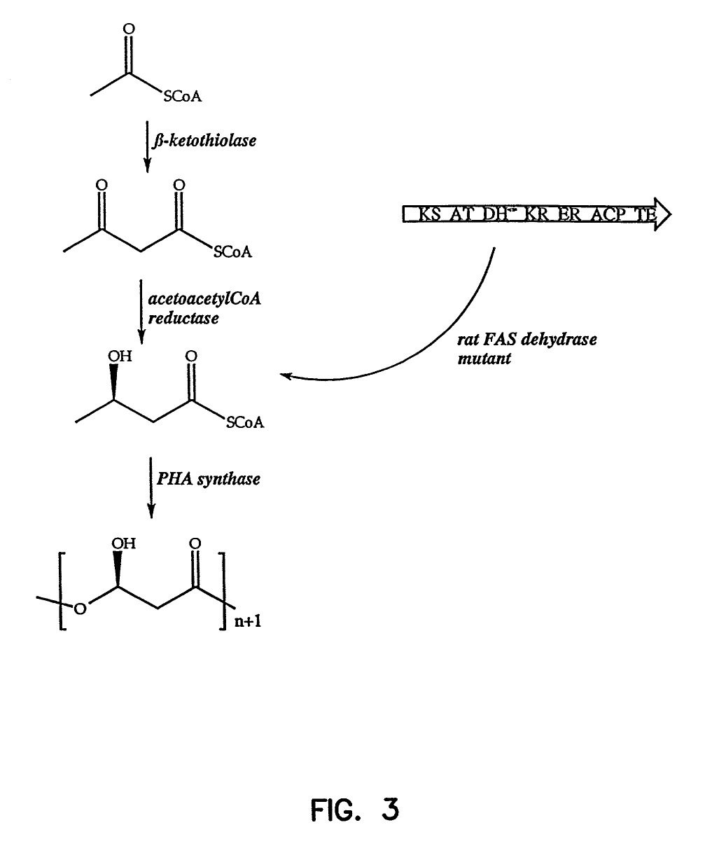

### DETAILED DESCRIPTION OF THE INVENTION

Definitions

As used herein, a “linker region” is an amino acid sequence present in a multifunctional protein which is less well conserved in an amino acid sequence than an amino acid sequence with catalytic activity.

As used herein, an “extender unit” catalytic or enzymatic domain is an acyl transferase in a module that catalyzes chain elongation by adding 2-4 carbon units to an acyl chain and is located carboxy-terminal to another acyl transferase. For example, an extender unit with methyhnalonylCoA specificity adds acyl groups to a methylmalonylCoA molecule.

As used herein, a “polyhydroxyalkanoate” or “PHA“polymer includes, but is not limited to, linked units of related, preferably heterologous, hydroxyalkanoates such as 3-hydroxybutyrate, 3-hydroxyvalerate, 3-hydroxycaproate, 3-hydroxyheptanoate, 3-hydroxyhexanoate, 3-hydroxyoctanoate, 3-hydroxyundecanoate, and 3-hydroxydodecanoate, and their 4-hydroxy and 5-hydroxy counterparts.

As used herein, a “Type I polyketide synthase” is a single polypeptide with a single set of iteratively used active sites. This is in contrast to a Type II polyketide synthase which employs active sites on a series of polypeptides.

As used herein, a “recombinant” nucleic acid or protein molecule is a molecule where the nucleic acid molecule which encodes the protein has been modified in vitro, so that its sequence is not naturally occurring, or corresponds to naturally occurring sequences that are not positioned as they would be positioned in a genome which has not been modified.

A “recombinant” host cell of the invention has a genome that has been manipulated in vitro so as to alter, e.g., decrease or disrupt, or, alternatively, increase, the function or activity of at least one gene in the macrolide or desosamine biosynthetic gene cluster of the invention.

As used herein, a “multiflnctional protein” is one where two or more enzymatic activities are present on a single polypeptide.

As used herein, a “module” is one of a series of repeated units in a multifunctional protein, such as a Type I polyketide synthase or a fatty acid synthase.

As used herein, a “premature termination product” is a product which is produced by a recombinant multifunctional protein which is different than the product produced by the non-recombinant multifunctional protein. In general, the product produced by the recombinant multifunctional protein has fewer acyl groups.

As used herein, a DNA that is “derived from” a gene cluster is a DNA that has been isolated and purified in vitro from genomic DNA, or synthetically prepared on the basis of the sequence of genomic DNA.

As used herein, the “pik” or “pik/met” gene cluster includes sequences encoding a polyketide synthase (pikA), desosamine biosynthetic enzymes (pikB, also referred to as des), a cytochrome P450 (pikC), regulatory factors (pikD) and enzymes for cellular self-resistance (pikR).

As used herein, the terms “isolated and/or purified” refer to in vitro isolation of a DNA or polypeptide molecule from its natural cellular environment, and from association with other components of the cell, such as nucleic acid or polypeptide, so that is can be sequenced, replicated and/or expressed. Moreover, the DNA may encode more than one recombinant Type I polyketide synthase and/or fatty acid synthase. For example, “an isolated DNA molecule encoding a polyhydroxyalkanoate monomer synthase” is RNA or DNA containing greater than 7, preferably 15, and more preferably 20 or more sequential nucleotide bases that encode a biologically active polypeptide, fragment, or variant thereof, that is complementary to the non-coding, or complementary to the coding strand, of a polyhydroxyalkanoate monomer synthase RNA, or hybridizes to the RNA or DNA encoding the polyhydroxyalkanoate monomer synthase and remains stably bound under stringent conditions, as defined by methods well known to the art, e.g., in Sambrook et al., supra.

An “antibiotic” as used herein is a substance produced by a microorganism which, either naturally or with limited chemical modification, will inhibit the growth of or kill another microorganism or eukaryotic cell.

An “antibiotic biosynthetic gene” is a nucleic acid, e.g., DNA, segment or sequence that encodes an enzymatic activity which is necessary for an enzymatic reaction in the process of converting primary metabolites into antibiotics.

An “antibiotic biosynthetic pathway” includes the entire set of antibiotic biosynthetic genes necessary for the process of converting primary metabolites into antibiotics. These genes can be isolated by methods well known to the art, e.g., see U.S. Pat. No. 4,935,340.

Antibiotic-producing organisms include any organism, including, but not limited to, Actinoplanes, Actinomadura, Bacillus, Cephalosporium, Micromonospora, Penicillium, Nocardia, and Streptomyces, which either produces an antibiotic or contains genes which, if expressed, would produce an antibiotic.

An antibiotic resistance-conferring gene is a DNA segment that encodes an enzymatic or other activity which confers resistance to an antibiotic.

The term “polyketide” as used herein refers to a large and diverse class of natural products, including but not limited to antibiotic, antifungal, anticancer, and anti-helminthic compounds. Antibiotics include, but are not limited to anthracyclines and macrolides of different types (polyenes and avermectins as well as classical macrolides such as erythromycins). Macrolides are produced by, for example, S. erytheus, S. antibioticus, S. venezuelae, S. fradiae and S. narbonensis.

The term “glycosylated polyketide” refers to any polyketide that contains one or more sugar residues.

The term “glycosylation-modified polyketide” refers to a polyketide having a changed glycosylation pattern or configuration relative to that particular polyketide's unmodified or native state.

The term “polyketide-producing microorganism” as used herein includes any microorganism that can produce a polyketide naturally or after being suitably engineered (i.e., genetically). Examples of actinomycetes that naturally produce polyketides include but are not limited to Micromonospora rosaria, Micromonospora megalomicea, Saccharopolyspora erythraea, Streptomyces antibioticus,, Streptomyces albereticuli, Streptomyces ambofaciens, Streptomyces avermitilis, Streptomyces fradiae, Streptomyces griseus, Streptomyces hydroscopicus, Streptomyces tsukulubaensis, Streptomyces mycarofasciens, Streptomyces platenesis, Streptomyces violaceoniger, Streptomyces violaceoniger, Streptomyces thermotolerans, Streptomyces rimosus, Streptomyces peucetius, Streptomyces coelicolor, Streptomyces glaucescens, Streptomyces roseofulvus, Streptomyces cinnamonensis, Streptomyces curacoi, and Amycolatopsis mediterranei (see Hopwood, D. A. and Sherman, D. H., Annu. Rev. Genet., 24:37-66 (1990), incorporated herein by reference). Other examples of polyketide-producing microorganisms that produce polyketides naturally include various Actinomadura, Dactylosporangium and Nocardia strains.

The term “sugar biosynthesis genes” as used herein refers to nucleic acid sequences from organisms such as Streptomyces venezuelae that encode sugar biosynthesis enzymes and is intended to include sequences of DNA from other polyketide-producing microorganisms which are identical or analogous to those obtained from Streptomyces venezuelae.

The term “sugar biosynthesis enzymes” as used herein refers to polypeptides which are involved in the biosynthesis and/or attachment of polyketide-associated sugars and their derivatives and intermediates.

The term “polyketide-associated sugar” refers to a sugar that is known to attach to polyketides or that can be attached to polyketides by the processes described herein.

The term “sugar derivative” refers to a sugar which is naturally associated with a polyketide but which is altered relative to the unmodified or native state, including but not limited to, N-3-α-desdimethyl D-desosamine.

The term “sugar intermediate” refers to an intermediate compound produced in a sugar biosynthesis pathway.

As used herein, the term “derivative” means that a particular compound produced by a host cell of the invention or prepared in vitro using polypeptides encoded by the nucleic acid molecules of the invention, is modified so that it comprises other moieties, e.g., peptide or polypeptide molecules, such as antibodies or fragments thereof, nucleic acid molecules, sugars, lipids, fats, a detectable signal molecule such as a radioisotope, e.g., gamma emitters, small chemicals, metals, salts, synthetic polymers, e.g., polylactide and polyglycolide, surfactants and glycosaminoglycans, which are covalently or non-covalently attached or linked to the compound.

A “recombinant” host cell of the invention has a genome that has been manipulated in vitro so as to alter, e.g., decrease or disrupt, or alternatively, increase, the function or activity of at least one gene, e.g., in the pik biosynthetic gene cluster, of the invention.

As used herein, the term “derivative” means that a particular compound produced by a host cell of the invention or prepared in vitro using polypeptides encoded by the nucleic acid molecules of the invention, is modified so that it comprises other moieties, e.g., peptide or polypeptide molecules, such as antibodies or fragments thereof, nucleic acid molecules, sugars, lipids, fats, a detectable signal molecule such as a radioisotope, e.g., gamma emitters, small chemicals, metals, salts, synthetic polymers, e.g., polylactide and polyglycolide, surfactants and glycosaminoglycans, which are covalently or non-covalently attached or linked to the compound.

It will be appreciated by those skilled in the art that each atom of the compounds of the invention having a chiral center may exist in and be isolated in optically active and racemic forms. Some compounds may exhibit polymorphism. It is to be understood that the present invention encompasses any racemic, optically active, polymorphic or stereoisomeric form, or mixtures thereof, of a compound of the invention, which possess the useful properties described herein, it being well known in the art how to prepare optically active forms (for example, by resolution of the racemic form by recrystallization techniques, by synthesis from optically active starting materials, by chiral synthesis, or by chromatographic separation using a chiral stationary phase) and how to determine activity using the standard tests described herein, or using other similar tests which are well known in the art.

The term “sequence homology” or “sequence identity” means the proportion of base matches between two nucleic acid sequences or the proportion amino acid matches between two amino acid sequences. When sequence homology is expressed as a percentage, e.g., 50%, the percentage denotes the proportion of matches over the length of sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete CDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

In accordance with the present invention there is provided an isolated and purified nucleic acid molecule which encodes the entire pathway for methymycin, pikromycin, neomethymycin, narbomycin, or a combination thereof, which includes sugar biosynthetic genes that are linked thereto. Desirably, the nucleic acid molecule is DNA isolated from Streptomyces spp. The present invention further includes isolated and purified nucleic acid sequences which hybridize under standard or stringent conditions to the nucleic acid molecules of the invention. It is also understood that the invention encompasses isolated and purified polypeptides which may be encoded by the nucleic acid molecules of the invention.

The invention described herein can be used for the production of a diverse range of novel compounds including polyketides, e.g., antibiotics, and biodegradable PHA polymers through genetic redesign of DNA encoding a FAS or a PKS such as that found in Streptomyces spp. Thus, the isolation and characterization of this gene cluster allows for the selective production of antibiotics, the overproduction or under production of particular compounds, e.g., overproduction of certain antibiotics, and the production of novel compounds. For example, combinational biosynthetic-based modification of compounds may be accomplished by selective activation or disruption of specific genes within the cluster or incorporation of the genes into biased biosynthetic libraries which are assayed for a wide range of biological activities, to derive greater chemical diversity. A further example includes the introduction of biosynthetic gene(s) into a particular host cell so as to result in the production of a novel compound due to the activity of the biosynthetic gene(s) on other metabolites, intermediates or components of the host cells.

Further, different PHA synthases can be tested for their ability to polymerize monomers produced by the recombinant PKS or PHA monomer synthase into a biodegradable polymer. The invention also provides a method by which various PHA synthases can be tested for their specificity with respect to different monomer substrates.

The potential uses and applications of PHAs produced by PHA monomer synthases and PHA synthases include both medical and industrial applications. Medical applications of PHAs include surgical pins, sutures, staples, swabs, wound dressings, blood vessel replacements, bone replacements and plates, stimulation of bone growth by piezoelectric properties, and biodegradable carrier for long-term dosage of pharmaceuticals. Industrial applications of PHAs include disposable items such as baby diapers, packaging containers, bottles, wrappings, bags, and films, and biodegradable carriers for long-term dosage of herbicides, fungicides, insecticides, or fertilizers.

In animals, the biosynthesis of fatty acids de novo from malonyl-CoA is catalyzed by FAS. For example, the rat FAS is a homodimer with a subunit structure consisting of 2505 amino acid residues having a molecular weight of 272,340 Da. Each subunit consists of seven catalytic activities in separate physical domains (Amy et al., Proc. Natl. Acad. Sci. USA, 6, 3114 (1989)). The physical location of six of the catalytic activities, ketoacyl synthase (KS), malonyl/acetyltransferase (M/AT), enoyl reductase (ER), ketoreductase (KR), acyl carrier protein (ACP), and thioesterase (TE), has been established by (1) the identification of the various active site residues within the overall amino acid sequence by isolation of catalytically active fragments from limited proteolytic digests of the whole FAS, (2) the identification of regions within the FAS that exhibit sequence similarity with various monofunctional proteins, (3) expression of DNA encoding an amino acid sequence with catalytic activity to produce recombinant proteins, and (4) the identification of DNA that does not encode catalytic activity, i.e., DNA encoding a linker region. (Smith et al., Proc. Natl. Acad. Sci. USA, 73, 1184 (1976); Tsukamoto et al., J. Biol. Chem., 263, 16225 (1988); Rangan et al., J. Biol. Chem., 226, 19180 (1991)).

The seventh catalytic activity, dehydrase (DH), was identified as physically residing between AT and ER by an amino acid comparison of FAS with the amino acid sequences encoded by the three open reading frames of the eryA polyketide synthase (PKS) gene cluster of Saccharopolyspora erythraea. The three polypeptides that comprise this PKS are constructed from “modules” which resemble animal FAS, both in terms of their amino acid sequence and in the ordering of the constituent domains (Donadio et al.,Gene, 111, 51 (1992); Benh et al., Eur. J. Biochem., 204, 39 (1992)).

One embodiment of the invention employs a FAS in which the DH is inactivated (FAS DH-). The FAS DH- employed in this embodiment of the invention is preferably a eukaryotic FAS DH- and, more preferably, a mammalian FAS DH-. The most preferred embodiment of the invention is a FAS where the active site in the DH has been inactivated by mutation. For example, Joshi et al. (J. Biol. Chem., 268, 22508 (1993)) changed the His⁸⁷⁸ residue in the rat FAS to an alanine residue by site-directed mutagenesis. In vitro studies showed that a FAS with this change (ratFAS206) produced 3-hydroxybutyrylCoA as a premature termination product from acetyl-CoA, malonyl-CoA and NADPH.

As shown below, a FAS DH- effectively replaces the β-ketothiolase and acetoacetyl-CoA reductase activities of the natural pathway by producing D(−)-3-hydroxybutyrate as a premature termination product, rather than the usual 16-carbon product, palmitic acid. This premature termination product can then be incorporated into PHB by a PHB synthase (See Example 2).

Another embodiment of the invention employs a recombinant Streptomyces spp. PKS to produce a variety of β-hydroxyCoA esters that can serve as monomers for a PHA synthase. One example of a DNA encoding a Type I PKS is the eryA gene cluster, which governs the synthesis of erythromycin aglycone deoxyerythronolide B (DEB). The gene cluster encodes six repeated units, termed modules or synthase units (SUs). Each module or SU, which comprises a series of putative FAS-like activities, is responsible for one of the six elongation cycles required for DEB formation. Thus, the processive synthesis of asymmetric acyl chains found in complex polyketides is accomplished through the use of a programmed protein template, where the nature of the chemical reactions occurring at each point is determined by the specificities in each SU.

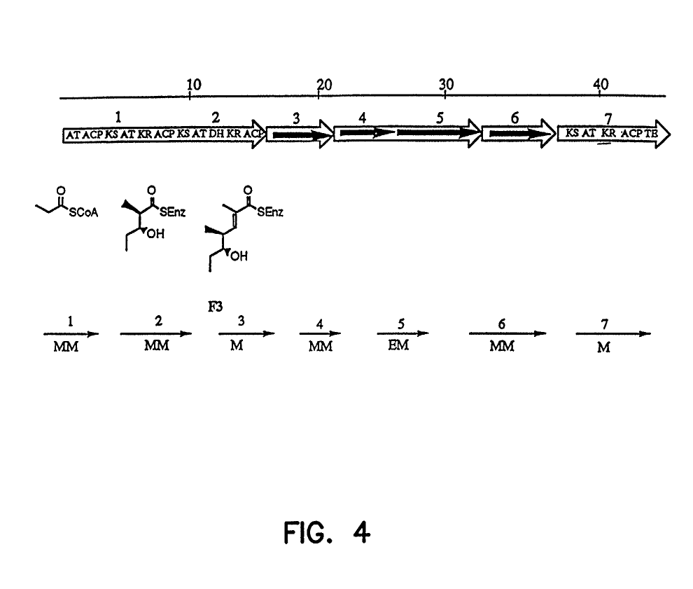

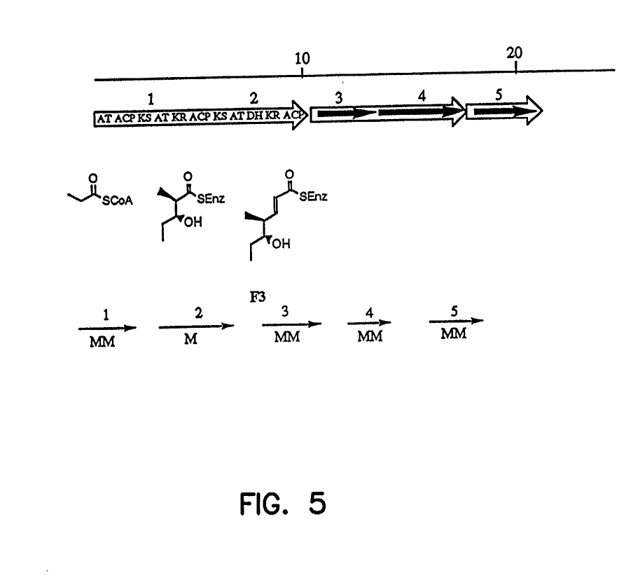

Two other Type I PKS are encoded by the tyl (tylosin) (FIG. 4) and met (methymycin) (FIG. 5) gene clusters. The macrolide multifunctional synthases encoded by tyl and met provide a greater degree of metabolic diversity than that found in the eryA gene cluster. The PKSs encoded by the eryA gene cluster only catalyze chain elongation with methylmalonylCoA, as opposed to tyl and met PKSs, which catalyze chain elongation with malonylCoA, methylmalonylCoA and ethylmalonylCoA. Specifically, the tyl PKS includes two malonylCoA extender units and one ethylmalonylCoA extender unit, and the met PKS includes one malonylCoA extender unit. Thus, a preferred embodiment of the invention includes, but is not limited to, replacing catalytic activities encoded in met PKS open reading frame 1 (ORF1) to provide a DNA encoding a protein that possesses the required keto group processing capacity and short-chain acylCoA ester starter and extender unit specificity necessary to provide a saturated β-hydroxyhexanoylCoA or unsaturated β-hydroxyhexenoylCoA monomer.

In order to manipulate the catalytic specificities within each module, DNA encoding a catalytic activity must remain undisturbed. To identify the amino acid sequences between the amino acid sequences with catalytic activity, the “linker regions,” amino acid sequences of related modules, preferably those encoded by more than one gene cluster, are compared. Linker regions are amino acid sequences which are less well conserved than amino acid sequences with catalytic activity. Witkowski et al., Eur. J. Biochem., 198, 571 (1991).

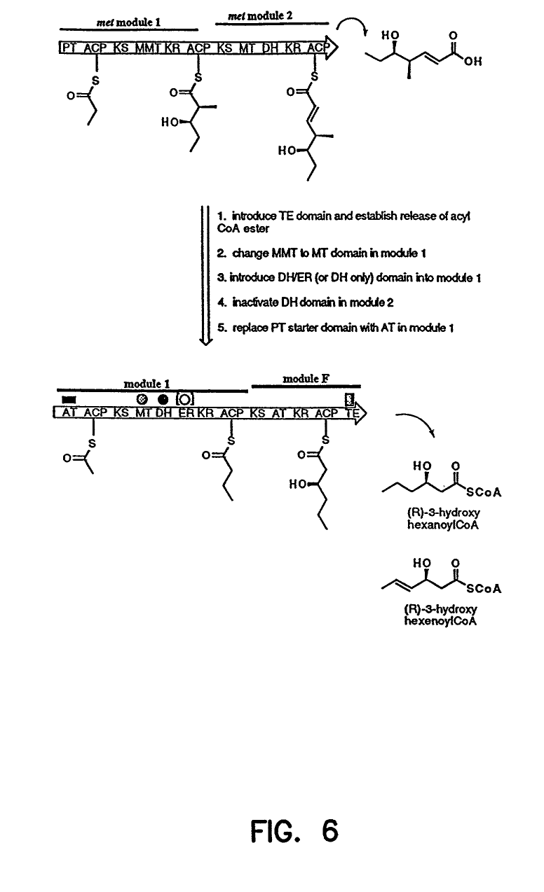

In an alternative embodiment of the invention, to provide a DNA encoding a Type I PKS module with a TE and lacking a functional DH, a DNA encoding a module F, containing KS, MT, KR, ACP, and TE catalytic activities, is introduced at the 3′ end of a DNA encoding a first module (FIG. 6). Module F introduces the final (R)-3-hydroxyl acyl group at the final step of PHA monomer synthesis, as a result of the presence of a TE domain. DNA encoding a module F is not present in the eryA PKS gene cluster (Donadio et al., supra, 1991).

A DNA encoding a recombinant monomer synthase is inserted into an expression vector. The expression vector employed varies depending on the host cell to be transformed with the expression vector. That is, vectors are employed with transcription, translation and/or post-translational signals, such as targeting signals, necessary for efficient expression of the genes in various host cells into which the vectors are introduced. Such vectors are constructed and transformed into host cells by methods well known in the art. See Sambrook et al., Molecular Cloning: A Laboratory Manual, Cold Spring Harbor (1989). Preferred host cells for the vectors of the invention include insect, bacterial, and plant cells. Preferred insect cells include Spodoptera frugiperda cells such as Sf21, and Trichoplusia ni cells. Preferred bacterial cells include Escherichia coli, Streptomyces and Pseudomonas. Preferred plant cells include monocot and dicot cells, such as maize, rice, wheat, tobacco, legumes, carrot, squash, canola, soybean, potato, and the like.

Moreover, the appropriate subcellular compartment in which to locate the enzyme in eukaryotic cells must be considered when constructing eukaryotic expression vectors. Two factors are important: the site of production of the acetyl-CoA substrate, and the available space for storage of the PHA polymer. To direct the enzyme to a particular subcellular location, targeting sequences may be added to the sequences encoding the recombinant molecules.

The baculovirus system is particularly amenable to the introduction of DNA encoding a recombinant FAS or a PKS monomer synthase because an increasing variety of transfer plasmids are becoming available which can accommodate a large insert, and the virus can be propagated to high titers. Moreover, insect cells are adapted readily to suspension culture, facilitating relatively large-scale recombinant protein production. Further, recombinant proteins tend to be produced exclusively as soluble proteins in insect cells, thus, obviating the need for refolding, a task that might be particularly daunting in the case of a large multifunctional protein. The Sf21/baculovirus system has routinely expressed milligram quantities of catalytically active recombinant fatty acid synthase. Finally, the baculovirus/insect cell system provides the ability to construct and analyze different synthase proteins for the ability to polymerize monomers into unique biodegradable polymers.

A further embodiment of the invention is the introduction of at least one DNA encoding a PHA synthase and a DNA encoding a PHA monomer synthase into a host cell. Such synthases include, but are not limited to, A. eutrophus 3-hydroxy, 4-hydroxy, and 5-hydroxy alkanoate synthases, Rhodococcus ruber C₃-C₅ hydroxyalkanoate synthases, Pseudomonas oleororans C₆-C₁₄ hydroxyalkanoate synthases, P. putida C₆-C₁₄ hydroxyalkanoate synthases, P. aeruginosa C₅-C₁₀ hydroxyalkanoate synthases, P. resinovorans 6l C₄-C₁₀ hydroxyalkanoate synthases, Rhodospirillum rubrum C₄-C₇ hydroxyalkanoate syntheses, R. gelatinorus C₄-C₇, Thiocapsa pfennigii C₄-C₈ hydroxyalkanoate synthases, and Bacillus megaterium C₄-C, hydroxyalkanoate synthases.

The introduction of DNA(s) encoding more than one PHA synthase may be necessary to produce a particular PHA polymer due to the specificities exhibited by different PHA synthases. As multifunctional proteins are altered to produce unusual monomeric structures, synthase specificity may be problematic for particular substrates. Although the A. eutrophus PHB synthase utilizes only C4 and C5 compounds as substrates, it appears to be a good prototype synthase for initial studies since it is known to be capable of producing copolymers of 3-hydroxybutyrate and 4-hydroxybutyrate (Kunioka et al., Macromolecules, 22, 694 (1989)) as well as copolymers of 3-hydroxyvalerate, 3-hydroxybutyrate, and 5-hydroxyvalerate (Doi et al., Macromolecules, 19, 2860 (1986)). Other synthases, especially those of Pseudomonas aeruginosa (Timm et al., Eur, J. Biochem., 209, 15 (1992)) and Rhodococcus ruber (Pieper et al., FEMS Microbiol. Lett., 96, 73 (1992)), can also be employed in the practice of the invention. Synthase specificity may be alterable through molecular biological methods.

In yet another embodiment of the invention, a DNA encoding a FAS and a PHA synthase can be introduced into a single expression vector, obviating the need to introduce the genes into a host cell individually.

A further embodiment of the invention is the generation of a DNA encoding a recombinant multifunctional protein, which comprises a FAS, of either eukaryotic or prokaryotic origin, and a PKS module F. Module F will carry out the final chain extension to include two additional carbons and the reduction of the β-keto group, which results in a (R)-3-hydroxy acyl CoA moiety.

To produce this recombinant protein, DNA encoding the FAS TE is replaced with a DNA encoding a linker region which is normally found in the ACP-KS interdomain region of bimodular ORFs. DNA encoding a module F is then inserted 3′ to the DNA encoding the linker region. Different linker regions, such as those described below which vary in length and amino acid composition, can be tested to determine which linker most efficiently mediates or allows the required transfer of the nascent saturated fatty acid intermediate to module F for the final chain elongation and keto reduction steps. The resulting DNA encoding the protein can then be tested for expression of long-chain β-hydroxy fatty acids in insect cells, such as Sf21 cells, or Streptomyces, or Pseudomonas. The expected 3-hydroxy C-18 fatty acid can serve as a potential substrate for PHA synthases which are able to accept long-chain alkyl groups. A preferred embodiment of the invention is a FAS that has a chain length specificity between 4-22 carbons.

Examples of linker regions that can be employed in this embodiment of the invention include, but are not limited to, the ACP-KS linker regions encoded by the tyl ORFI (ACP₁-KS₂; ACP₂-KS₃), and ORF3 (ACP₅-KS₆), and eryA ORFI (ACP₁-KS₁; ACP₂-KS₂), ORF2 (ACP₃-KS₄) and ORF3 (ACP₅-KS₆).

This approach can also be used to produce shorter chain fatty acid groups by limiting the ability of the FAS unit to generate long-chain fatty acids. Mutagenesis of DNA encoding various FAS catalytic activities, starting with the KS, may result in the synthesis of short-chain (R)-3-hydroxy fatty acids.

The PHA polymers are then recovered from the biomass. Large-scale solvent extraction can be used, but is expensive. An alternative method involving heat shock with subsequent enzymatic and detergent digestive processes is also available (Byron, Trends Biotechnical, 5, 246 (1987); Holmes, In: Developments in Crystalline Polymers, D. C. Bassett (ed.), pp. 1-65 (1988)). PHB and other PHAs are readily extracted from microorganisms by chlorinated hydrocarbons. Refluxing with chloroform has been extensively used; the resulting solution is filtered to remove debris and concentrated, and the polymer is precipitated with methanol or ethanol, leaving low-molecular-weight lipids in solution. Longer side-chain PHAs show a less restricted solubility than PHB and are, for example, soluble in acetone. Other strategies adopted include the use of ethylene carbonate and propylene carbonate as disclosed by Lafferty et al. (Chem. Rundschau, 30, 14 (1977)) to extract PHB from biomass. Scandola et al. (int. J. Biol. Microbiol., 10, 373 (1988)) reported that 1 M HCl-chloroform extraction of Rhizobium meliloti yielded PHB of M_{w}=6×10⁴ compared with 1.4×10⁶ when acetone was used.

Methods are well known in the art for the determination of the PHB or PHA content of microorganisms, the composition of PHAs, and the distribution of the monomer units in the polymer. Gas chromatography and high-pressure liquid chromatography are widely used for quantitative PHB analysis. See Anderson et al., Microbiol. Rev., 54, 450 (1990) for a review of such methods. NMR techniques can also be used to determine polymer composition, and the distribution of monomer units.

Preparation of Variant Nucleic Acid Molecules and Variant Polypeptides of the Invention

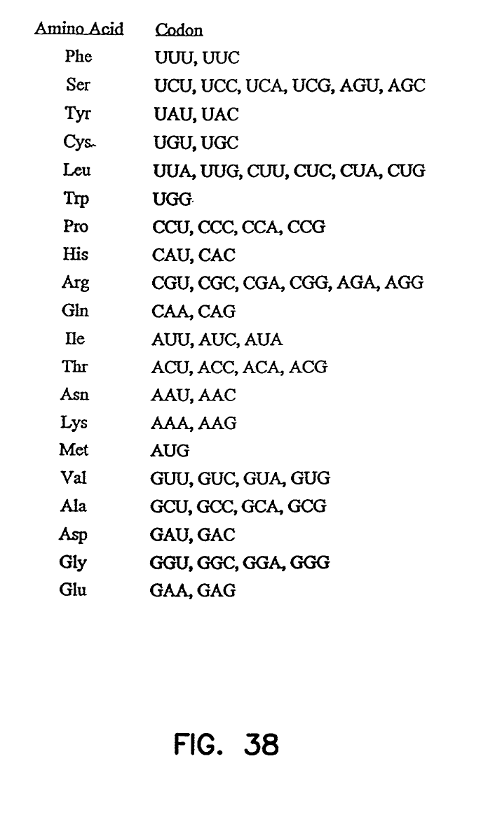

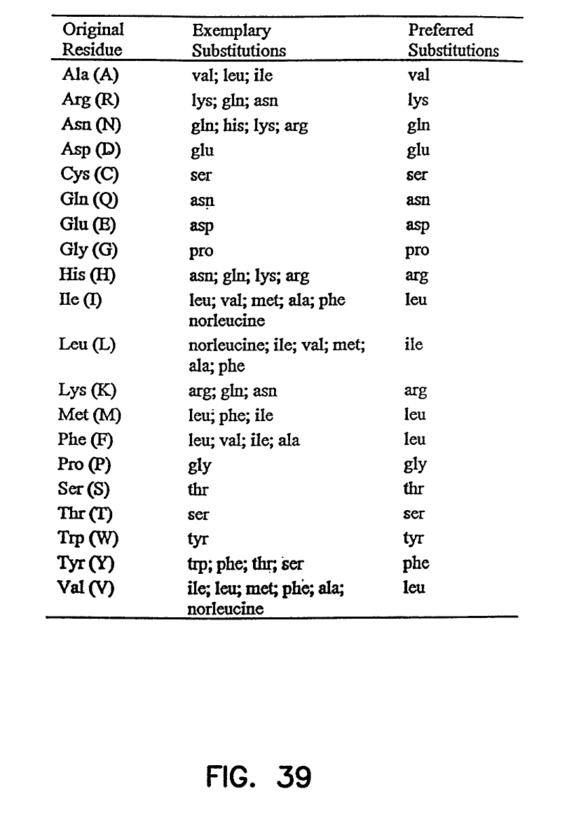

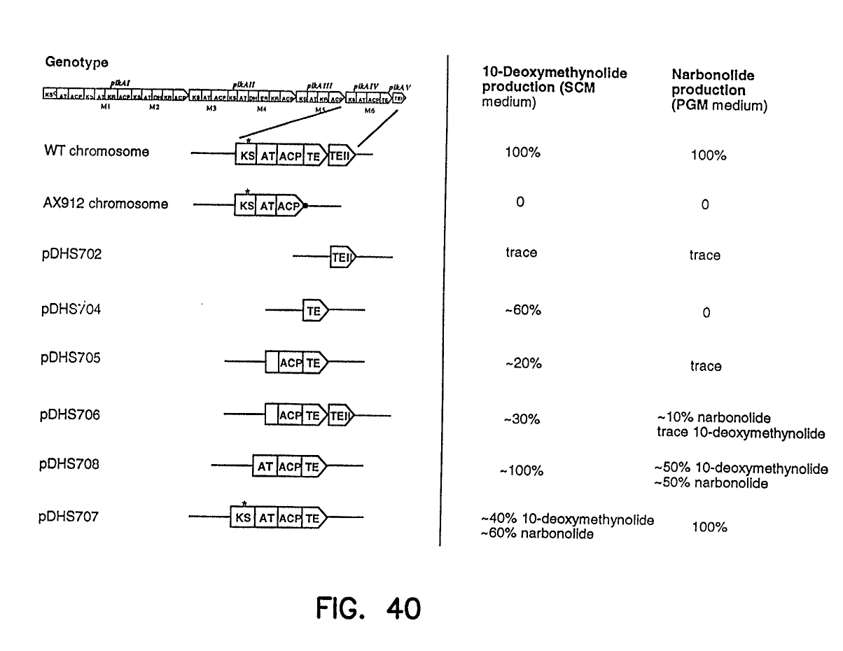

The present invention also contemplates nucleic acid sequences which hybridize under stringent hybridization conditions to the nucleic acid sequences set forth herein. Stringent hybridization conditions are well known in the art and define a degree of sequence identity greater than about 80 to about 90%. Thus, nucleic acid sequences encoding variant polypeptides (FIG. 38), or nucleic acid sequences having conservative (silent) nucleotide substitutions (FIG. 37), are within the scope of the invention. Preferably, variant polypeptides encoded by the nucleic acid sequences of the invention are biologically active. The present invention also contemplates naturally occurring allelic variations and mutations of the nucleic acid sequences described herein.

As is well known in the art, because of the degeneracy of the genetic code, there are numerous other DNA and RNA molecules that can code for the same polypeptides as those encoded by the exemplified biosynthetic genes and fragments thereof. The present invention, therefore, contemplates those other DNA and RNA molecules which, on expression, encode the polypeptides of, for example, portions of SEQ ID NO:4 or SEQ ID NO:6. Having identified the amino acid residue sequence encoded by a sugar biosynthetic or macrolide biosynthetic gene, and with knowledge of all triplet codons for each particular amino acid residue, it is possible to describe all such encoding RNA and DNA sequences. DNA and RNA molecules other than those specifically disclosed herein and, which molecules are characterized simply by a change in a codon for a particular amino acid, are within the scope of this invention.

The 20 common amino acids and their representative abbreviations, symbols and codons are well known in the art (see, for example, Molecular Biology of the Cell, Second Edition, B. Alberts et al., Garland Publishing Inc., New York and London, 1989). As is also well known in the art, codons constitute triplet sequences of nucleotides in mRNA molecules and as such, are characterized by the base uracil (U) in place of base thymidine (T) which is present in DNA molecules. A simple change in a codon for the same amino acid residue within a polynucleotide will not change the structure of the encoded polypeptide. By way of example, it can be seen from SEQ ID NO:6 that a TCT codon for serine exists at nucleotide positions 1735-1737. However, it can also be seen from that same sequence that serine can be encoded by a TCA codon (see, e.g., nucleotide positions 1738-1740) and a TCC codon (see, e.g., nucleotide positions 1874-1876). Substitution of the latter codons for serine with the TCT codon for serine or vice versa, does not substantially alter the DNA sequence of SEQ ID NO:6 and results in production of the same polypeptide. In a similar manner, substitutions of the recited codons with other equivalent codons can be made in a like manner without departing from the scope of the present invention.

A nucleic acid molecule, segment or sequence of the present invention can also be an RNA molecule, segment or sequence. An RNA molecule contemplated by the present invention corresponds to, is complementary to or hybridizes under stringent conditions to any of the DNA sequences set forth herein. Exemplary and preferred RNA molecules are mRNA molecules that encode sugar biosynthetic or macrolide biosynthetic enzymes of this invention.

Mutations can be made to the native nucleic acid sequences of the invention and such mutants used in place of the native sequence, so long as the mutants are able to function with other sequences to collectively catalyze the synthesis of an identifiable polyketide or macrolide. Such mutations can be made to the native sequences using conventional techniques such as by preparing synthetic oligonucleotides including the mutations and inserting the mutated sequence into the gene using restriction endonuclease digestion. (See, e.g., Kunkel, T. A. Proc. Natl. Acad, Sci. USA (1985) 82:448; Geisselsoder et al. BioTechniques (1987) 5:786.) Alternatively, the mutations can be effected using a mismatched primer (generally 10-20 nucleotides in length) which hybridizes to the native nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. Zoller and Smith, Methods Enzymol., (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al., Proc. Natl. Acad. Sci. USA (1982) 79:6409. PCR mutagenesis will also find use for effecting the desired mutations.

Random mutagenesis of the nucleotide sequence can be accomplished by several different techniques known in the art, such as by altering sequences within restriction endonuclease sites, inserting an oligonucleotide linker randomly into a plasmid, by irradiation with X-rays or ultraviolet light, by incorporating incorrect nucleotides during in vitro DNA synthesis, by error-prone PCR mutagenesis, by preparing synthetic mutants or by damaging plasmid DNA in vitro with chemicals. Chemical mutagens include, for example, sodium bisulfite, nitrous acid, hydroxylamine, agents which damage or remove bases thereby preventing normal base-pairing such as hydrazine or formic acid, analogues of nucleotide precursors such as nitrosoguanidine, 5-bromouracil, 2-aminopurine, or acridine intercalating agents such as proflavine, acriflavine, quinacrine, and the like. Generally, plasmid DNA or DNA fragments are treated with chemicals, transformed into E. coli and propagated as a pool or library of mutant plasmids.

Large populations of random enzyme variants can be constructed in vivo using “recombination-enhanced mutagenesis.”This method employs two or more pools of, for example, 10⁶ mutants each of the wild-type encoding nucleotide sequence that are generated using any convenient mutagenesis technique and then inserted into cloning vectors.

The gene sequences can be inserted into one or more expression vectors, using methods known to those of skill in the art. Expression vectors may include control sequences operably linked to the desired genes. Suitable expression systems for use with the present invention include systems which function in eukaryotic and prokaryotic host cells. Prokaryotic systems are preferred, and in particular, systems compatible with Streptomyces spp. are of particular interest. Control elements for use in such systems include promoters, optionally containing operator sequences, and ribosome binding sites. Particularly usefuil promoters include control sequences derived from the gene clusters of the invention. However, other bacterial promoters, such as those derived from sugar metabolizing enzymes, such as galactose, lactose (lac) and maltose, will also find use in the expression cassettes encoding desosamine. Preferred promoters are Streptomyces promoters, including but not limited to the ermE*, pikA, and tipA promoters. Additional examples include promoter sequences derived from biosynthetic enzymes such as tryptophan (trp), the β-lactamase (bla) promoter system, bacteriophage lambda PL, and T5. In addition, synthetic promoters, such as the tac promoter (U.S. Pat. No. 4,551,433), which do not occur in nature, also function in bacterial host cells.

Other regulatory sequences may also be desirable which allow for regulation of expression of the genes relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes which confer antibiotic resistance or sensitivity to the plasmid. Alternatively, several polyketides are naturally colored and this characteristic provides a built-in marker for selecting cells successfully transformed by the present constructs.

The various subunits of interest can be cloned into one or more recombinant vectors as individual cassettes, with separate control elements, or under the control of, e.g., a single promoter. The subunits can include flanking restriction sites to allow for the easy deletion and insertion of other subunits so that hybrid PKSs can be generated. The design of such unique restriction sites is known to those of skill in the art and can be accomplished using the techniques described above, such as site-directed mutagenesis and PCR.

For sequences generated by random mutagenesis, the choice of vector depends on the pool of mutant sequences, i.e., donor or recipient, with which they are to be employed. Furthermore, the choice of vector determines the host cell to be employed in subsequent steps of the claimed method. Any transducible cloning vector can be used as a cloning vector for the donor pool of mutants. It is preferred, however, that phagemids, cosmids, or similar cloning vectors be used for cloning the donor pool of mutant encoding nucleotide sequences into the host cell. Phagemids and cosmids, for example, are advantageous vectors due to the ability to insert and stably propagate therein larger fragments of DNA than in M13 phage and λ phage, respectively. Phagemids which will find use in this method generally include hybrids between plasmids and filamentous phage cloning vehicles. Cosmids which will find use in this method generally include λ phage-based vectors into which cos sites have been inserted. Recipient pool cloning vectors can be any suitable plasmid. The cloning vectors into which pools of mutants are inserted may be identical or may be constructed to harbor and express different genetic markers (see, e.g., Sambrook et al., supra). The utility of employing such vectors having different marker genes may be exploited to facilitate a determination of successful transduction.

Thus, for example, the cloning vector employed may be an E. coli/Streptomyces shuttle vector (see, for example, U.S. Pat. Nos. 4,416,994, 4,343,906, 4,477,571, 4,362,816, and 4,340,674), a cosmid, a plasmid, an artificial bacterial chromosome (see, e.g., Zhang and Wing, Plant Mol. Biol., 35, 115 (1997); Schalkwyk et al., Curr. Op. Biotech., 6, 37 91995); and Monaco and Lavin, Trends in Biotech., 12, 280 (1994), or a phagemid, and the host cell may be a bacterial cell such as E. coli, Penicillium patulum, and Streptomyces spp. such as S. lividans, S. venezuelae , or S. lavendulae, or a eukaryotic cell such as fungi, yeast or a plant cell, e.g., monocot and dicot cells, preferably cells that are regenerable.

Moreover, recombinant polypeptides having a particular activity may be prepared via “gene-shuffling”. See, for example, Crameri et al., Nature, 391, 288 (1998); Patten et al., Curr. Op. Biotech., 8, 724 (1997), U.S. Pat. Nos. 5,837,458, 5,834,252, 5,830,727, 5,811,238, 5,605,793).

For phagemids, upon infection of the host cell which contains a phagemid, single-stranded phagemid DNA is produced, packaged and extruded from the cell in the form of a transducing phage in a manner similar to other phage vectors. Thus, clonal amplification of mutant encoding nucleotide sequences carried by phagemids is accomplished by propagating the phagemids in a suitable host cell.

Following clonal amplification, the cloned donor pool of mutants is infected with a helper phage to obtain a mixture of phage particles containing either the helper phage genome or phagemids mutant alleles of the wild-type encoding nucleotide sequence.

Infection, or transfection, of host cells with helper phage is generally accomplished by methods well known in the art (see., e.g., Sambrook et al., supra; and Russell et al. (1986) Gene 45:333-338).

The helper phage may be any phage which can be used in combination with the cloning phage to produce an infective transducing phage. For example, if the cloning vector is a cosmid, the helper phage will necessarily be a λ phage. Preferably, the cloning vector is a phagemid and the helper phage is a filamentous phage, and preferably phage M13.

If desired after infecting the phagemid with helper phage and obtaining a mixture of phage particles, the transducing phage can be separated from helper phage based on size difference (Barnes et al. (1983) Methods Enzymol. 101:98-122), or other similarly effective technique.

The entire spectrum of cloned donor mutations can now be transduced into clonally amplified recipient cells into which has been transduced or transformed a pool of mutant encoding nucleotide sequences. Recipient cells which may be employed in the method disclosed and claimed herein may be, for example, E. coli, or other bacterial expression systems which are not recombination deficient. A recombination deficient cell is a cell in which recombinatorial events is greatly reduced, such as rec- mutants of E. coli (see, Clark et al. (1965) Proc. Natl. Acad. Sci. USA 53:451-459).

These transductants can now be selected for the desired expressed protein property or characteristic and, if necessary or desirable, amplified. Optionally, if the phagemids into which each pool of mutants is cloned are constructed to express different genetic markers, as described above, transductants may be selected by way of their expression of both donor and recipient plasmid markers.

The recombinants generated by the above-described methods can then be subjected to selection or screening by any appropriate method, for example, enzymatic or other biological activity.

The above cycle of amplification, infection, transduction, and recombination may be repeated any number of times using additional donor pools cloned on phagemids. As above, the phagemids into which each pool of mutants is cloned may be constructed to express a different marker gene. Each cycle could increase the number of distinct mutants by up to a factor of 10^{6.} Thus, if the probability of occurrence of an inter-allelic recombination event in any individual cell is f (a parameter that is actually a function of the distance between the recombining mutations), the transduced culture from two pools of 10⁶ allelic mutants will express up to 10¹² distinct mutants in a population of 10¹²/f cells.

The invention will be further described by the following non-limiting examples.

### I. Experimental Procedures

Materials and Methods

Materials. Sodium R-(−)-3-hydroxybutyrate, coenzyme-A, ethylchloroforrnate, pyridine and diethyl ether were purchased from Sigma Chemical Co. Amberlite IR-120 was purchased from Mallinckrodt Inc. 6-O-(N-Heptylcarbamoyl)methyl α-D-glycopyranoside (Hecameg) was obtained from Vegatec (Villeejuif, France). Two-piece spectrophotometer cells with pathlengths of 0.1 (#20/0-Q-1) and 0.01 cm (#20/0-Q-0.1) were obtained from Stama Cells Inc. (Atascadero, Calif.). Rabbit anti-A. eutrophus PHA synthase antibody was a gracious gift from Dr. F. Srienc and S. Stoup (Biological Process Technology Institute, University of Minnesota). Sf21 cells and T. ni cells were kindly provided by Greg Franzen (R&D Systems, Minneapolis, Minn.) and Stephen Harsch (Department of Veterinary Pathobiology, University of Minnesota), respectively.

Plasmid pFAS206 and a recombinant baculoviral clone encoding FAS206 (Joshi et al., J. Biol. Chem., 268, 22508 (1993)) were generous gifts of A. Joshi and S. Smith. Plasmid pAet41(Peoples et al., J. Biol. Chem., 264, 15298 (1989)), the source of theA. eutrophus PHB synthase, was obtained from A. Sinskey. Baculovirus transfer vector, pBacPAK9, and linearized baculoviral DNA, were obtained from Clontech Inc. (Palo Alto, Calif.). Restriction enzymes, T4 DNA ligase, E. coli DH5α competent cells, molecular weight standards, lipofectin reagent, Grace's insect cell medium, fetal bovine serum (FBS), and antibiotic/antimycotic reagent were obtained from GIBCO-BRL (Grand Island, N.Y.). Tissue culture dishes were obtained from Coming Inc. Spinner flasks were obtained from Bellco Glass Inc. Seaplaque agarose GTG was obtained from FMC Bioproducts Inc.

Methods

Preparation of R-3HBCoA. R-(−)-3 HBCoA was prepared by the mixed anhydride method described by Haywood et al., FEMS Microbiol. Lett., 57, 1(1989). 60 mg (0.58 nmol) of R-(−)-3 hydroxybutyric acid was freeze dried and added to a solution of 72 mg of pyridine in 10 ml diethyl ether at 0° C. Ethylchloroformate (100 mg) was added, and the mixture was allowed to stand at 4° C. for 60 minutes. Insoluble pyridine hydrochloride was removed by centrifugation. The resulting anhydride was added, dropwise with mixing, to a solution of 100 mg coenzyme-A (0.13 mmol) in 4 ml 0.2 M potassium bicarbonate, pH 8.0 at 0° C. The reaction was monitored by the nitroprusside test of Stadtman, Meth., Enzynol., 3, 931 (1957), to ensure sufficient anhydride was added to esterify all the coenzyme-A. The concentration of R-3-HBCoA was determined by measuring the absorbance at 260 nm (e=16.8 nM¹ cm¹; 18).

Construction of pBP-phbC. The phbC gene (approximately 1.8 kb) was excised from pAet41(Peoples et al., J. Biol. Chem., 264, 15293 (1989)) by digestion with BstBI and StuI, purified as described by Williams et al. (Gene, 109, 445 (1991)), and ligated to pBacPAK9 digested with BstBI and StuI. This resulted in pBP-phbC, the baculovirus transfer vector used in formation of recombinant baculovirus particles carrying phbC.

Large-scale expression of PHA synthase. A 1 L culture of T. ni cells (1.2×10⁶ cells/ml) in logarithmic growth was infected by the addition of 50 ml recombinant viral stock solution (2.5×10⁸ pfu/ml) resulting in a multiplicity of infection (MOI) of 10. This infected culture was split between two Bellco spinners (350 ml/500 ml spinner, 700 ml/1 L spinner) to facilitate oxygenation of the culture. These cultures were incubated at 28° C. and stirred at 60 rpm for 60 hours. Infected cells were harvested by centrifugation at 1000×g for 10 minutes at 4° C. Cells were flash frozen in liquid N₂ and stored in 4 equal aliquots, at −80° C. until purification.

Insect cell maintenance and recombinant baculovirus formation. Sf21 cells were maintained at 26-28° C. in Grace's insect cell medium supplemented with 10% FBS, 1.0% pluronic F68, and 1.0% antibiotic/antimycotic (GIBCO-BRL). Cells were typically maintained in suspension at 0.2-2.0×10⁶/ml in 60 ml total culture volume in 100 ml spinner flasks at 55-65 rpm. Cell viability during the culture period was typically 95-100%. The procedures for use of the transfer vector and baculovirus were essentially those described by the manufacturer (Clontech, Inc.). Purified pBP-phbC and linearized baculovirus DNA were used for cotransfection of Sf21 cells using the liposome-mediated method (Felgner et al., Proc. Natl. Acad. Sci. USA, 84, 7413 (1987)) utilizing Lipofectin (GIBCO-BRL). Four days later cotransfection supernatants were utilized for plaque purification. Recombinant viral clones were purified from plaque assay plates containing 1.5% Seaplaque GTG after 5-7 days at 28° C. Recombinant viral clone stocks were then amplified in T25-flask cultures (4 ml, 3×10⁶/ml on day 0) for 4 days; infected cells were determined by their morphology and size and then screened by SDS/PAGE using 10% polyacrylamide gels (Laemmli, Nature, 222, 680 (1970)) for production of PHA synthase.

Purification of PHA synthase from BTI-TN-5BI-4 T. ni cells. Purification of PHA synthase was performed according to the method of Gerngross et al., Biochemistry 33, 9311 (1994) with the following alterations. One aliquot (110 mg protein) of frozen cells was thawed on ice and resuspended in 10 mM KPi (pH 7.2), 5% glycerol, and 0.05% Hecameg (Buffer A) containing the following protease inhibitors at the indicated final concentrations: benzarnidine (2 mM), phenylmethylsulfonyl fluoride (PMSF, 0.4 mM), pepstatin (2 mg/ml), leupeptin (2.5 mg/ml), and Na-p-tosyl-l-lysine chloromethyl ketone (TLCK, 2 mM). EDTA was omitted at this stage due to its incompatibility with hydroxylapatite (HA). This mixture was homogenized with three series of 10 strokes each in two Thomas homogenizers while partially submerged in an ice bath and then sonicated for 2 minutes in a Branson Sonifier 250 at 30% cycle, 30% power while on ice. All subsequent procedures were carried out at 4° C.

The lysate was immediately centrifuged at 100000×g in a Beckman 50.2 Ti rotor for 80 minutes, and the resulting supernatant (10.5 ml, 47 mg) was immediately filtered through a 0.45 mm Uniflow filter (Schleicher and Schuell Inc., Keene, N.H.) to remove any remaining insoluble matter. Aliquots of the soluble fraction (1.5 ml, 7 mg) were loaded onto a 5 ml BioRad Econo-Pac HTP column that had been equilibrated with Buffer A (+ protease inhibitor mix) attached to a BioRad Econo-system, and the column was washed with 30 ml Buffer A. All chromatographic steps were carried out at a flow rate of 0.8 ml/minute. PHA synthase was eluted form the HA column with a 32×32 ml linear gradient from 10 to 300 mM KPi.

Fraction collection tubes were prepared by addition of 30 ml of 100 mM EDTA to provide a metalloprotease inhibitor at 1 mM immediately after HA chromatography. PHA synthase was eluted in a broad peak between 110-180 mM KPi. Fractions (3 ml) containing significant PHA synthase activity were pooled and stored at 0° C. until the entire soluble fraction had been run through the chromatographic process. Pooled fractions then were concentrated at 4° C. by use of a Centriprep-30 concentrator (Amicon) to 3.8 mg/ml. Aliquots (0.5 ml) were either flash frozen and stored in liquid N₂ or glycerol was added to a final concentration of 50% and samples (1.9 mg/ml) were stored at −20° C.

Western analysis. Samples of T. ni cells were fractionated by SDS-PAGE on 10% polyacrylamide gels, and the proteins then were transferred to 0.2 mm nitrocellulose membranes using a BioRad Transblot SD Semi-Dry electrophoretic transfer cell according to the manufacturer. Proteins were transferred for 1 hour at 15 V. The membrane was rinsed with doubly distilled H₂O, dried, and treated with phosphate-buffered saline (PBS) containing 0.05% Tween-20 (PBS-Tween) and 3% nonfat dry milk to block non-specific binding sites. Primary antibody (rabbit anti-PHA synthase) was applied in fresh blocking solution and incubated at 25° C. for 2 hours. Membranes were then washed four times for 10 minutes with PBS-Tween followed by the addition of horseradish peroxidase-conjugated goat-anti-rabbit antibody (Boehringer-Mannheim) diluted 10,000× in fresh blocking solution and incubated at 25° C. for 1 hour. Membranes were washed finally in three changes (10 minutes) of PBS, and the immobilized peroxidase label was detected using the chemiluminescent LumiGLO substrate kit (Kirkegaard and Perry, Gaithersburg, Md.) and X-ray film.

N-ternninal analysis. Approximately 10 mg of purified PHA synthase was run on a 10% SDS-polyacrylamide gel, transferred to PVDF (Immobilon-PSQ, Millipore Corporation, Bedford, Mass.), stained with Amido Black, and sequenced on a 494 Procise Protein Sequencer (Perkin-Elmer, Applied Biosystems Division, Foster City, Calif.).

Double-infection protocol. Four 100 ml spinner flasks were each inoculated with 8×10⁷ cells in 50 ml of fresh insect medium. To flask 1, an additional 20 ml of fresh insect medium was added (uninfected control); to flask 2, 10 ml BacPAK6::phbC viral stock (1×10⁸ pfu/ml) and 10 ml fresh insect medium were added; to flask 3, 10 ml BacPAK6::FAS206 viral stock (1×10⁸ pfu/ml) and 10 ml fresh insect medium were added; and to flask 4, 10 ml BacPAK6::phbC viral stock (1×10⁸ pf/ml) and 10 ml BacPAK6::FAS206 viral stock (1×10⁸ pfu/ml) were added. These viral infections were carried out at a multiplicity of infection of approximately 10. Cultures were maintained under normal growth conditions and 15 ml samples were removed at 24, 48, and 72 hour time points. Cells were collected by gentle centrifuigation at 1000×g for 5 minutes, the medium was discarded, and the cells were immediately stored at −70° C.

PHA synthase assays. Coenzyme A released by PHA synthase in the process of polymerization was monitored precisely as described by Gerngross et al. (supra) using 5,5′-dithiobis (2-nitrobenzoic acid, DTNB) (Ellman, Arch. Biochem. Biophys., 82, 70 (1959)).

The presence of HBCoA was monitored spectrophotometrically. Assays were performed at 25° C. in a Hewlett Packard 8452A diode array spectrophotometer equipped with a water-jacketed cell holder. Two-piece Starna Spectrosil spectrophotometer cells with pathlengths of 0.1 and 0.01 cm were employed to avoid errors arising from the compression of the absorbance scale at higher values. Absorbance was monitored at 232 nm, and E₂₃₂ nm of 4.5×103 M⁻¹ cm⁻¹ was used in calculations. One unit (U) of enzyme is the amount required to hydrolyze 1 mmol of substrate minute⁻¹. Buffer (0.15 M KPi, pH 7.2) and substrate were equilibrated to 25° C. and then combined in an Eppendorf tube also at 25° C. Enzyme was added and mixed once in the pipet tip used to transfer the entire mixture to the spectrophotometer cell. The two-piece cell was immediately assembled, placed in the spectrophotometer with the cell holder (type CH) adapted for the standard 10 mm pathlength cell holder of the spectrophotometer. Manipulations of sample, from mixing to initiation of monitoring, took only 10-15 seconds. Absorbance was continually monitored for up to 10 minutes. Calibration of reactions was against a solution of buffer and enzyme (no substrate) which led to absorbance values that represented substrate only.

PHBs assay. PHB was assayed from Sf21 cell samples according to the propanolysis method of Riis et al., J. Chromo., 445, 285 (1988). Cell pellets were thawed on ice, resuspended in 1 ml cold ddH₂O and transferred to 5 ml screwtop test tubes with teflon seals. Two ml of ddH₂O were added, the cells were washed and centrifuged and then 3 ml of acetone were added and the cells washed and centrifuged. The samples were then desiccated by placing them in a 94° C. oven for 12 hours. The following day 0.5 ml of 1,2-dichloroethane, 0.5 ml acidified propanol (20 ml HCl, 80 ml 1-propanol) and 50 ml benzoic acid standard were added and the sealed tubes were heated to 100° C. in a boiling water bath for 2 hours with periodic vortexing. The tubes were cooled to room temperature and the organic phase was used for gas-chromatographic (GC) analysis using a Hewlett Packard 5890A gas chromatograph equipped with a Hewlett Packard 7673A automatic injector and a fused silica capillary column, DB-WAX 30W of 30 meter length. Positive samples were further subjected to GC-mass spectrometric (MS) analysis for the presence of propylhydroxybutyrate using a Kratos MS25 GC/MS. The following parameters were used: source temperature, 210° C.; voltage, 70 eV; and accelerating voltage, 4 KeV.

Catalytic activities

Ketoacyl synthase (KS) activity was assessed radiochemically by the condensation-¹⁴CO₂ exchange reaction (Smith et al., PNAS USA, 73, 1184 (1976)).

Transferase (AT) activity was assayed, using malonyl-CoA as donor and pantetheine as acceptor, by determining spectrophotometrically the free CoA released in a coupled ATP citrate-lyase-malate dehydrogenase reaction (see, Rangen et al., J. Biol. Chem., 266, 19180 (1991).

Ketoreductase (KR) was assayed spectrophotometrically at 340 nm: assay systems contained 0.1 M potassium phosphate buffer (pH 7), 0.15 mM NADPH, enzyme and either 10 mM trans-1-decalone or 0.1 mM acetoacetyl-CoA substrate.

Dehydrase (DH) activity was assayed spectrophotometrically at 270 nm using S-DL-β-hydyroxybutyryl N-acetylcysteamine as substrate (Kumar et al., J. Biol, Chem., 245, 4732 (1970)).

Enoyl reductase (ER) activity was assayed spectrophotometrically at 340 nm essentially as described by Strom et al. (J. Biol, Chem, 254, 8159 (1979)); the assay system contained 0.1 M potassium phosphate buffer (pH 7), 0.15 mM NADPH, 0.375 nM crotonoyl-CoA, 20 μM CoA and enzyme.

Thioesterase (TE) activity was assessed radiochemically by extracting and assaying the [¹⁴C]palmitic acid formed from [1-¹⁴C]palmitoyl-CoA during a 3 minute incubation Smith, Meth. Enzymol., 71C, 181 (1981); the assay was in a final volume of 0.1 ml, 25 mM potassium phosphate buffer (pH 8), 20 μM [1-¹⁴C]palmitoyl-CoA (20 nCi) and enzyme.

Assay of overall fatty acid synthase activity was performed spectrophotometrically as described previously by Smith et al. (Meth. Enzymol., 35, 65 (1975)). All enzyme activities were assayed at 37° C. except the transferase, which was assayed at 20° C. Activity units indicate umol of substrate consumed/minute. All assays were conducted, at a minimum, at two different protein concentrations with the appropriate enzyme and substrate blanks included.

### II. EXAMPLES

### Example 1

### Expression of A. Eutrophus PHA Synthase Using a Baculovirus System

Recent work has shown that PHA synthase from A. eutrophus can be overexpressed in E. coli, in the absence of 3-ketothiolase and acetoacetyl-CoA reductase (Gerngross et al., supra) and can be expressed in plants (See Poirier et al., Biotech, 13, 142 (1995) for a review). Isolation of the soluble form of PHA synthase provides opportunities to examine the mechanistic details of the priming and initiation reactions. Because the baculovirus system has been successful for the expression of a number of prokaryotic genes as soluble proteins, and insect cells, unlike bacterial expression systems, carry out a wide array of post-translational modifications, the baculovirus expression system appeared ideal for the expression of large quantities of soluble PHA synthase, a protein that must be modified by phosphopantetheine in order to be catalytically active (Gerngross et al., supra).

Purification of PHA synthase. The purification procedure employed for PHA synthase is a modification of Gerngross et al. (supra) involving the elimination of the second liquid chromatographic step and inclusion of a protease-inhibitor cocktail in all buffers. All steps were carried out on ice or at 4° C. except where noted. Frozen cells were thawed on ice in 10 ml of Buffer A (10 mM KPi, pH 7.2, 05% glycerol, and 0.05% Hecameg) and then immediately homogenized prior to centrifugation and HA chromatography.

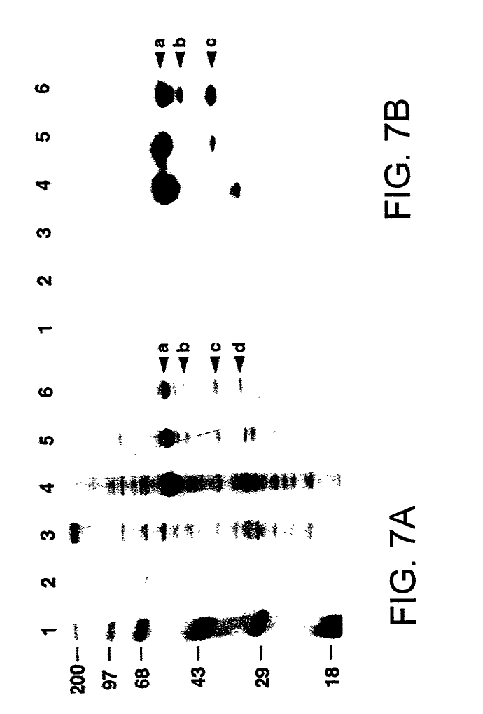

The results of these efforts are summarized in Table 1 and FIG. 7. A prominent band at 64 kDa is visible in total, soluble, and HA eluate protein samples fractionated by SDS/PAGE (lanes 4, 5, and 6 of FIG. 7, respectively). The initial specific activity of the isolated PHA synthase was 20-fold higher than previous attempts at expression and purification of this polypeptide. Approximately 1000 units of PHB synthase have been purified, based on calculations from the direct spectrophotometric assay detailed below, with an overall recovery of activity of 70%. The large proportion of synthase present in the membrane fraction, and the fact that over 90% of the initial activity was found in the soluble fraction, suggest either that the synthase in the membrane fraction is in an inactive form or that the direct assay is not applicable to the initial, 12 U/mg, crude extract.

TABLE 1
Purification of PHA Synthase

| | | | | protein specific | | |
|---|---|---|---|---|---|---|
| sample | total units | vol (mL) | (mg) | (mg/ml) | activity | recovery |
| total | 1430 | 11.5 | 113 | 9.8 | 12.7 | 100 |
| protein | | | | | | |
| soluble | 1340 | 10.5 | 47 | 4.5 | 28.6 | 93 |
| protein | | | | | | |
| pooled | 1020 | 7.9 | 30 | 3.8 | 34.2 | 71 |
| HA | | | | | | |
| fractions | | | | | | |

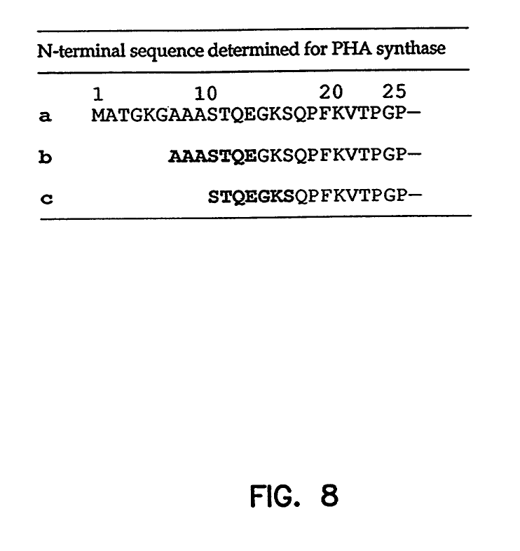

N-terminal sequencing of the 64 kDa protein confirmed its identity as PHA synthase (FIG. 8). Two prominent N-termini, at amino acid residue 7 (alanine) and residue 10 (serine) were obtained in a 3:2 ratio. This heterogeneous N-terninus presumably is the result of aminopeptidase activity. Western analysis using a rabbit-anti-PHA synthase antibody corroborated the results of the sequencing and indicated the presence of at least three bands that resulted from proteolysis of PHA synthase (FIG. 7B, lanes 4-6). The antibody was specific for PHA synthase since neither T. ni nor baculoviral proteins showed reactivity (FIG. 7B, lanes 2 and 3). N-terminal protein sequencing (FIG. 8) showed directly that the 44 kDa (band b) and 32 kDa (band d) proteins were derived from PHA synthase (fragments beginning at A181/N185 and at G387, respectively). The 35-40 kDa (band c) protein gave low sequencing yields and may contain a blocked N-terminus. Inspection of FIG. 7B suggests that most degradation occurs following cell disruption since the total protein sample of this gel (lane 4) was prepared by boiling intact cells directly in SDS sample buffer while the HA sample (lane 6) went through the purification procedure described above.

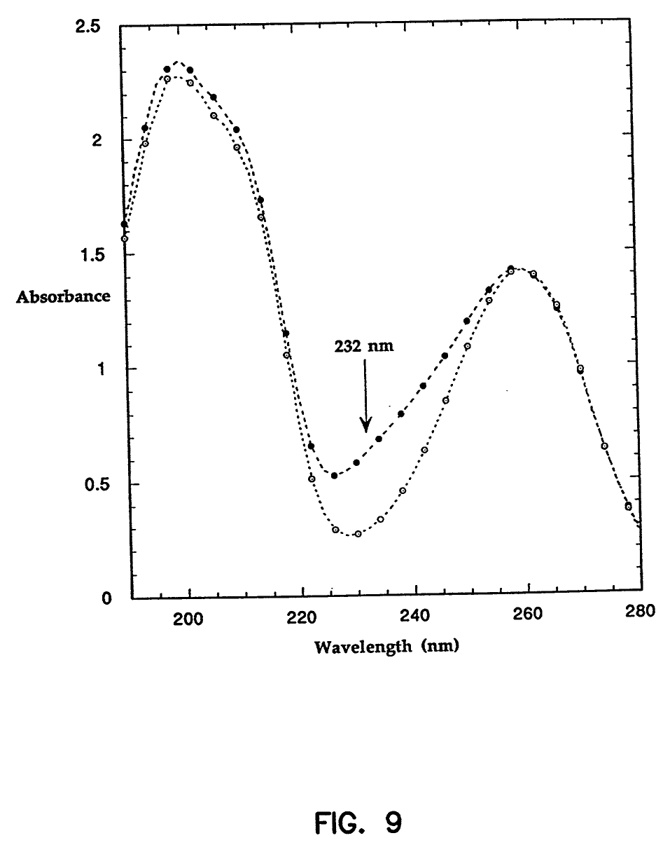

Assay of synthase activity. Due to the significant level of expression obtained using the baculovirus system, the synthase activity could be assayed spectrophotometrically by monitoring hydrolysis of the thioester bond at 232 nm, the wavelength at which there is a maximum decrease in absorbance upon hydrolysis. The difference between substrate (HBCoA) and product (CoA) at this wavelength is shown in FIG. 9. Absorbance of HBCoA and CoA at 232 nm occurs at a trough between two well-separated peaks. Assays were carried out at pH 7.2 for comparative analysis with previous studies (Gerngross et al., supra). Substrate (R-(−)3-HBCoA) substrate for these studies was prepared using the mixed anhydride method (Haywood et al., supra), and its concentration was determined by measuring A₂₆₀. The short pathlength cells (0.1 cm and 0.01 cm) allowed use of relatively high reaction concentrations while conserving substrate and enzyme. Assay results showed an initial lag period of 60 seconds prior to the linear decrease in A₂₃₂, and velocities were determined from the slope of these linear regions of the assay curves. The length of the lag period was variable and was inversely related to enzyme concentration. These data are consistent with those using PHA synthase purified from E. coli (Gerngross et al., supra).

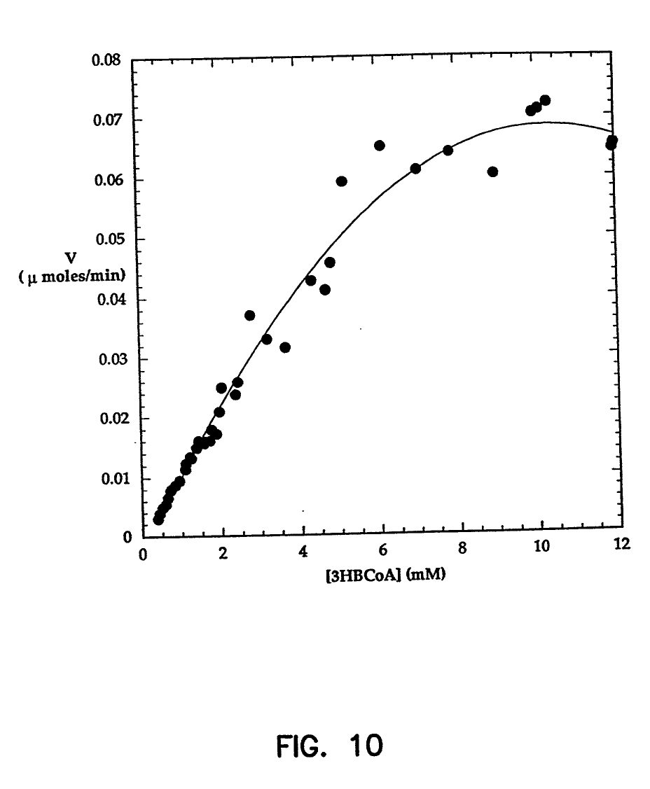

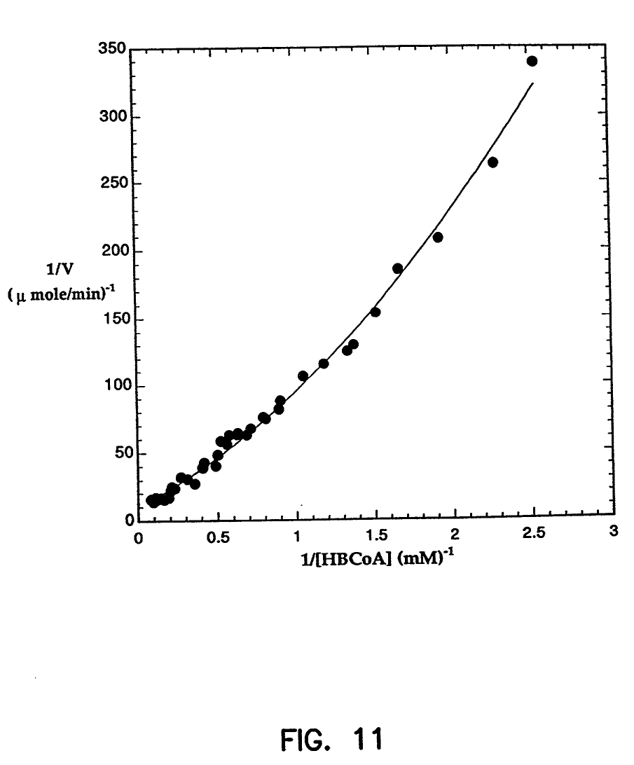

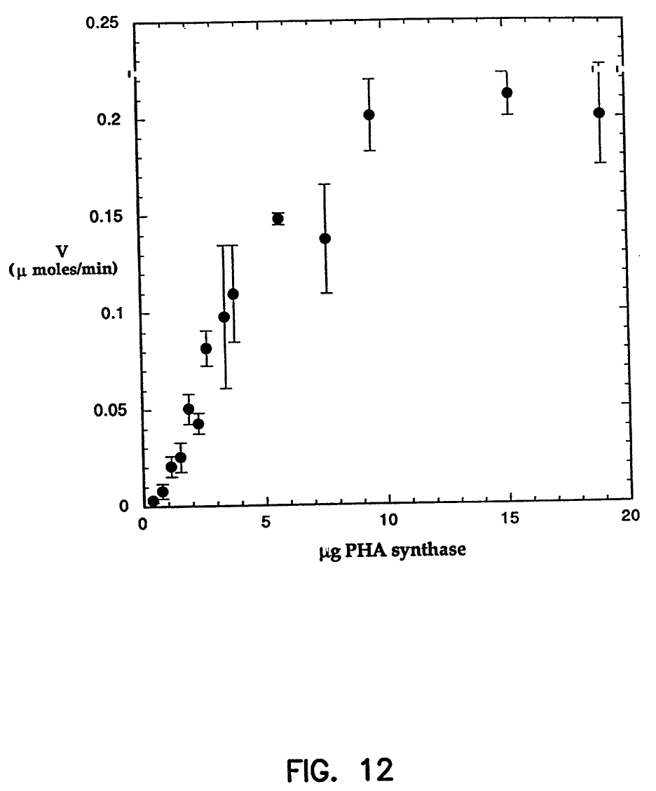

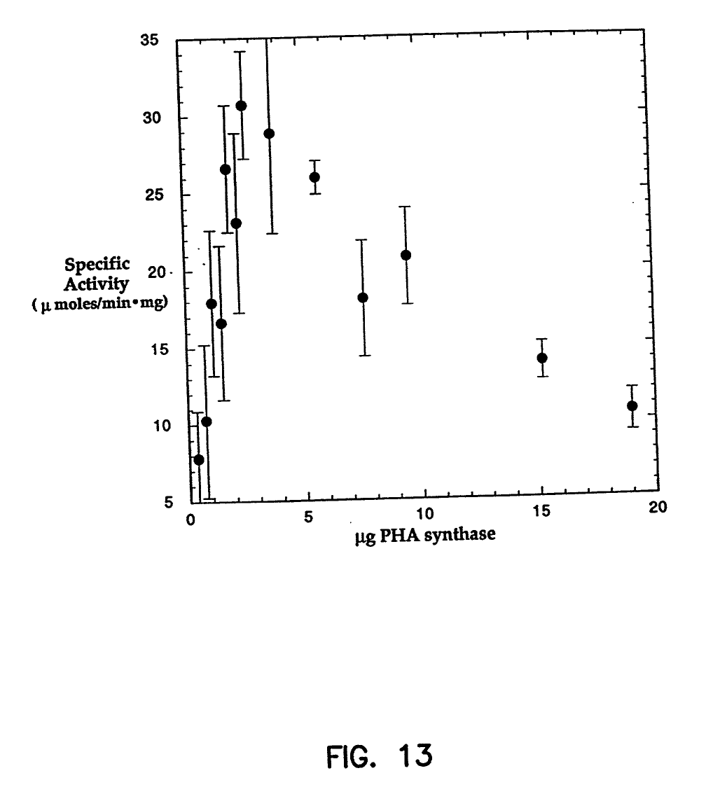

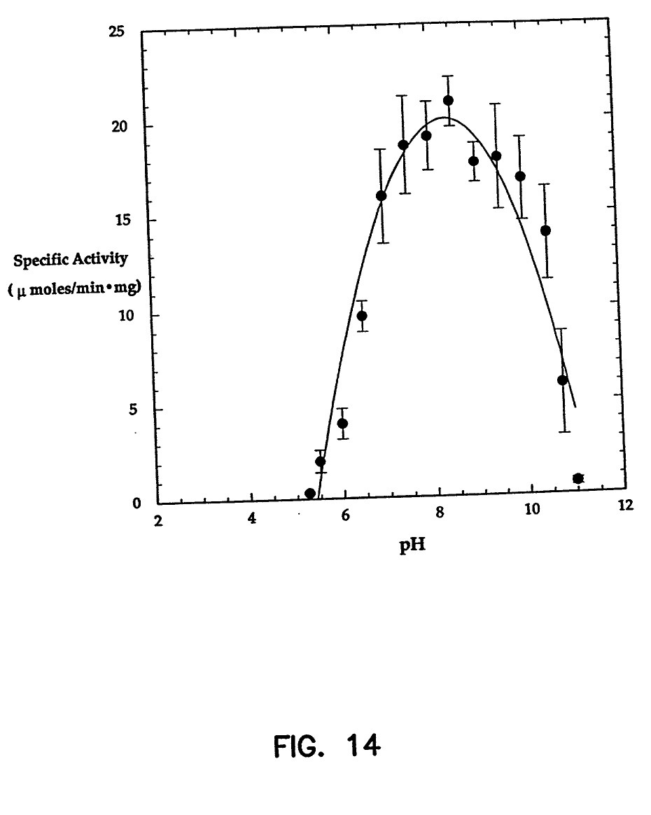

FIGS. 10 and 11 show the V versus S and 1/V versus 1/S plots, respectively. The double reciprocal plot was concave upward which is similar to results obtained from studies of the granular PHA synthase from Zooglea ramigera (Fukui et al., Arch. Microbiol., 110, 149 (1976)) and suggests a complex reaction mechanism. Examinations of velocity and specific activity as a function of enzyme concentration are shown in FIGS. 12 and 13. These results confirm that specific activity of the synthase depends upon enzyme concentration. The pH activity curve for A. eutrophus PHA synthase purified from T. ni cells is shown in FIG. 14. The curve shows a broad activity maximum centered around pH 8.5. This result agrees well with prior work on the A. eutrophus PHB synthase although it is significantly different than results obtained for the PHB synthase from Z. ramigera for which the optimum was determined to be pH 7.0.

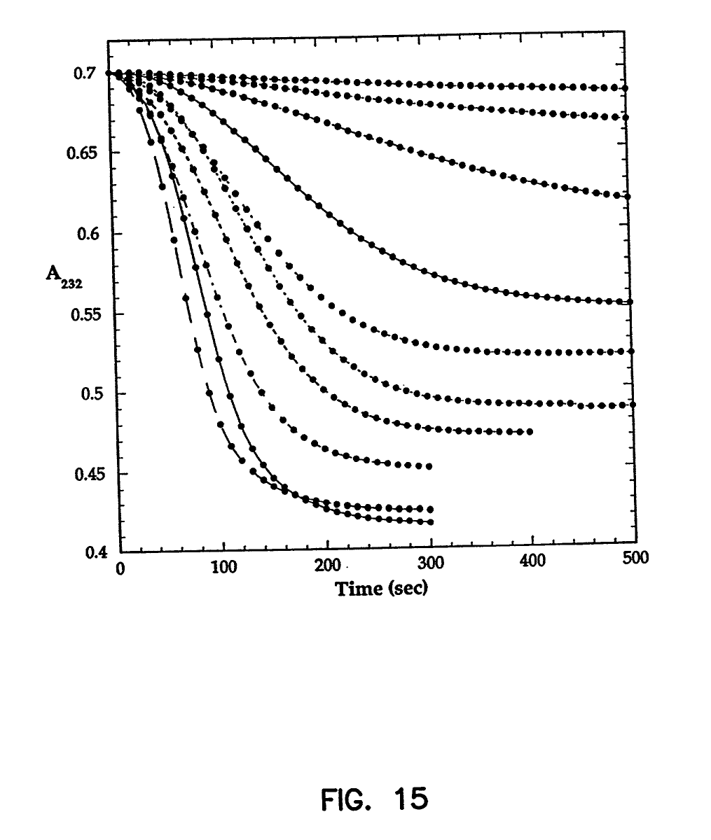

The effect of varying enzyme concentration in the presence of a fixed amount of substrate revealed an intriguing trend (FIG. 15). From these data it appears that the extent of polymerization is dependent on the amount of enzyme included in the reaction mixture. This could be explained if there is a “terminal length” limitation of the polymer, which, once reached, cannot be extended any further. If this is the case, it would also suggest that termination of the polymerization reaction, the release of the synthase from the polymer, and/or reinitiation of polymerization by the newly released synthase are relatively slow events since no evidence of these reactions are seen within the time course of these studies. The phenomenon observed in FIG. 15 is not the result of decay of the enzyme over the course of the assay since virtually identical results are obtained following a 10 minute preincubation of the synthase at 25° C.

It must also be noted that comparisons of the direct spectrophotometric assays used here and the more common assay involving the use of Ellman's reagent, DTNB, (Ellman, supra) in the formation of thiolate of coenzyme-A showed that the values determined by the direct method were approximately 70% of the values determined using Ellman's reagent. This may be due to phase separation occurring in the cuvettes as the relatively insoluble polymer is formed. In support of this notion, a faint haze or opalescence in the cuvette developed during the course of the reaction, particularly at higher substrate concentrations.

PHA synthase purified from insect cells appears to be relatively stable. Examination of activity following storage, in liquid N₂ and at −20° C. in the presence of 50% glycerol showed that approximately 50% of synthase activity remained after 7 weeks when stored in liquid N₂ and approximately 75% of synthase activity remained after 7 weeks when stored at −20° C. in the presence of 50% glycerol.

The expression of PHA synthase from A. eutrophus in a baculovirus expression system results in the synthase constituting approximately 50% of total protein 60 hours post-infection; however, approximately 50-75% of the synthase is observed in the membrane-associated fraction. This elevated level of expression allowed purification of the soluble PHA synthase using a single chromatographic step on HA. The purity of this preparation is estimated to be approximately 90% (intact PHA synthase and 3 proteolysis products).

The initial specific activity of 12 U/mg was approximately 20-fold higher than the most successful previous efforts at overexpression of A. eutrophus PHA synthase. The synthase reported here was isolated from a 250 ml culture with 70% recovery which represents an improvement of 500-fold (1000 U /64 U×8 L /0.25 L) when compared to an 8 L E. coli culture with 40% recovery. This high expression level should provide sufficient PHA synthase for extensive structural, fuictional, and mechanistic studies. Furthermore, it is clear that the baculovirus expression system is an attractive option for isolation of other PHA synthases from various sources.

PHA synthase produced in the baculovirus system was of sufficient potency to allow direct spectrophotometric analysis of the hydrolysis of the thioester bond of HBCoA at 232 nm. These assays revealed a lag period of approximately 60 seconds, the length of which was variable and inversely related to enzyme concentration. Such a lag period presumably reflects a slow step in the reaction, perhaps correlating to dimerization of the enzyme, the priming, and/or initiation steps in formation of PHB. Size exclusion chromatographic examination of the PHB synthase native MW indicated two forms of the synthase. One form showed a MW of approximately 100-160 kDa and the other showed a MW of approximately 50-80 kDA; these two forms likely represent the dimer and monomer of PHA synthase, respectively. Similar results have been reported previously in which two forms of approximately 60 and 130 kDa were observed. Comparisons of the direct assay reported here and the indirect assay using DTNB revealed that the former resulted in values that were 70% of the values determined by the DTNB indirect assay. Although the reason for this difference has not been examined in detail, it is probable that the apparent phase separation that occurred upon PHB formation in the short pathlength cuvettes used, particularly with high [HBCoA], results in this discrepancy.

Enzymatic analyses of the PHA synthase have found that the enzyme has a broad pH optimum centered at pH 8.5; however, the studies described herein have been performed at pH 7.2 to provide comparative values with the results of others. Moreover, the specific activity of this enzyme is dependent upon enzyme concentration which confirms and extends earlier results (Gerngross et al., supra).

In studies intended to examine the dependence of activity upon enzyme concentration, it became apparent that the extent of the polymerization reaction is dependent on the amount of enzyme included in the reaction mixture. Specifically, decreasing the amount of enzyme leads not only to decreased velocity of reaction but also to a decreased extent of condensation (FIG. 15). One possible explanation is that the enzyme is thermally labile; however, identical assays in which the enzyme is preincubated at 25° C. for 10 minutes prior to initiation of the reaction had similar results. Another possibility is that a terminal-length of the polymer is reached precluding further condensations until the particular synthase molecule is released from the terminal-length polymer.

This work clearly demonstrates the value of the baculovirus expression system for the production of A. eutrophus PHA synthase and for the potential application to studies of other PHA synthases. Furthermore, the high level of expression obtained using the baculoviral system should allow convenient analysis for substrate-specificity and structure-function studies of PHA synthases from relatively crude insect cell extracts.

### Example 2

### Co-expression of Rat FAS Dehydrase Mutant cDNA and PHB Synthase Gene in Insect Cells

Expression of a rat FAS DH- cDNA in Sf9 cells has been reported previously (Rangan et al., J. Biol. Chem, 266, 19180 (1991); Joshi et al., Biochem, J., 226, 143 (1993)). Once activity of the phbC gene product had been established in insect cells (see Example 1), baculovirus clones containing the rat FAS DH- cDNA and BacPAK6:.phbC were employed in a double-infection strategy to determine if PHB would be produced in insect cells. It was not known if an intracellular pool of R(−)-3-hydroxybutyrate would be stable or available as a substrate for the PHB synthase. In order for the R-(−)-3-hydroxybutyrylCoA to be available as a substrate, the R-(−)-3-hydroxybutyrylCoA released from rat FAS DH- protein must be trapped by the PHB synthase and incorporated into a polymer at a rate faster than β-oxidation, which would regenerate acetylCoA. It was also not known if the stereochemical configuration of the 3-hydroxyl group, which must be in the R form, would be recognized as a substrate by PHB synthase. Fortunately, previous biochemical studies on eukaryotic FASs indicated that the R form of 3-hydroxybutyrylCoA would be generated (Wakil et al., J. Biol. Chem., 237, 687 (1962)).

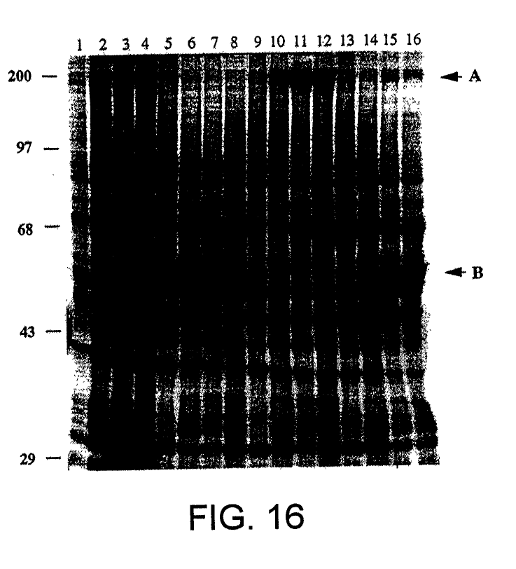

SDS-PAGE of protein samples from a time course of uninfected, single-infected, and dual-infected Sf21 cells was performed (FIG. 16). From these data, it is clear that the rat FAS DH mutant and PHB synthase polypeptides are efficiently co-expressed in Sf21 cells. However, co-expression results in ˜50% reduced levels of both polypeptides compared to Sf21 cells that are producing the individual proteins. Western analysis using anti-rat FAS (Rangan et al., supra) and anti-PHA synthase antibodies confirmed simultaneous production of the corresponding proteins.

To provide further evidence that PHB was being synthesized in insect cells, T. ni cells which had been infected with a baculovirus vector encoding rat FAS DH⁰ and/or a baculovirus vector encoding PHA synthase were analyzed for the presence of granules. Infected cells were fixed in paraformaldehyde and incubated with anti-PHA synthase antibodies (Williams et al., Protein Exp. Purif., 7, 203 (1996)). Granules were observed only in doubly infected cells (Williams et al., App. Environ. Micro., 62 2540 (1996)).

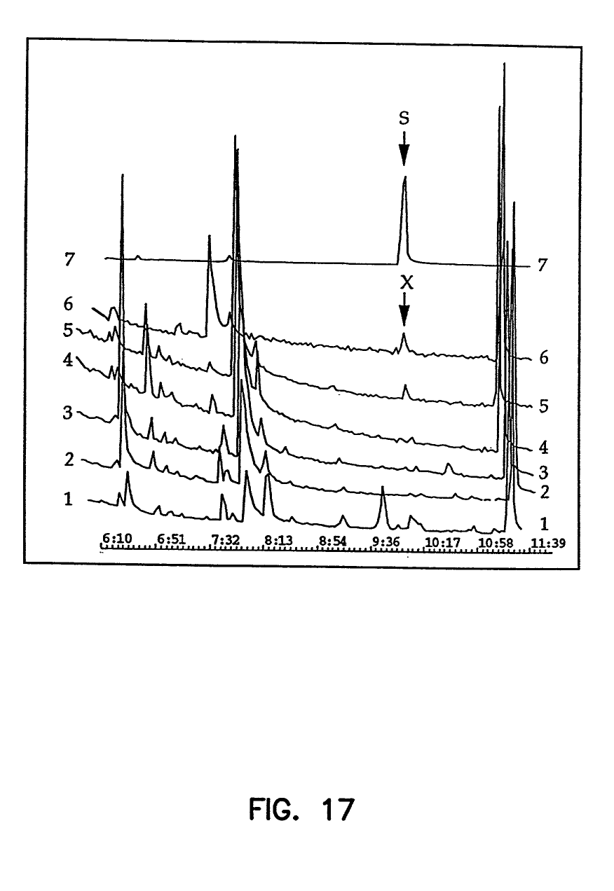

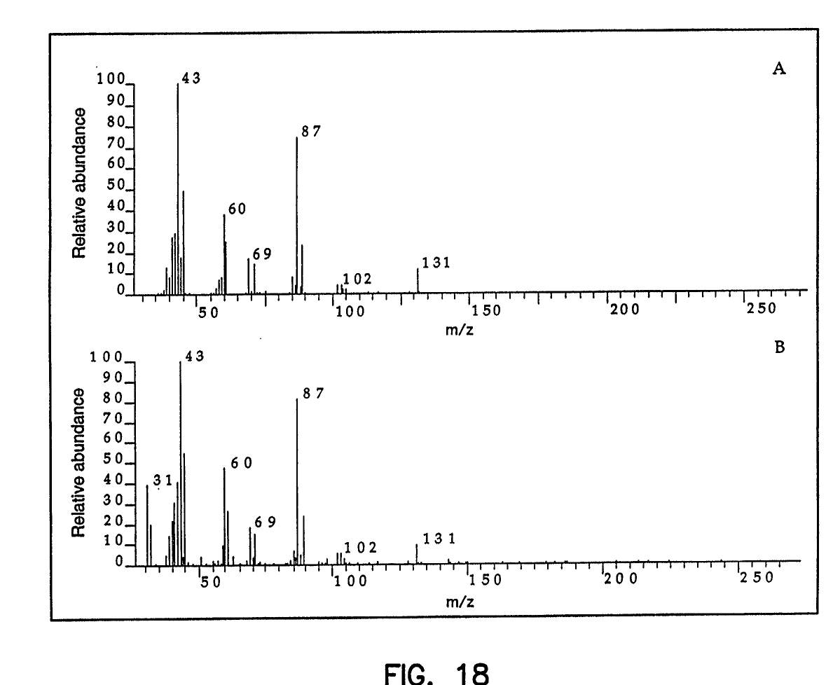

Characterization of PHB production in insect cells. In order to determine if de novo synthesis of PHB was occurring in Sf21 cells that co-express the rat FAS DH mutant and PHB synthase, fractions of these samples were extracted, the extract subjected to propanolysis, and analyzed for the presence of propylhydroxybutyrate by gas chromatography (FIG. 17). A unique peak with a retention time that coincided with a propylhydroxybutyrate standard was detected only in the double infection samples at 48 and 72 hours, in contrast to the individually expressed gene products and uninfected controls, which were negative. These samples were analyzed further by GC/MS to confirm the identity of the product. FIG. 18 shows mass spectroscopy data corresponding to the material obtained from peak 10.1 in the gas chromatograph compared to a propylhydroxybutyrate standard. The results show that PHB synthesis is occurring only in Sf21 cells co-expressing the rat FAS DH mutant cDNA and the phbC gene from A. eutrophus. Integration of the peak in the gas chromatograph corresponding to propylhydroxybutyrate revealed that approximately 1 mg of PHB was isolated from 1 liter culture of Sf21 cells (approximately 600 mg dry cell weight of Sf21 cells). Thus, the ratFAS206 protein effectively replaces the β-ketothiolase and acetoacetyl-CoA reductase functions, resulting in the production of PHB by a novel pathway.

The approach described here provides a new strategy to combine metabolic pathways that are normally engaged in primary anabolic functions for production of polyesters. The premature termination of the normal fatty acid biosynthetic pathway to provide suitably modified acylCoA monomers for use in PHA synthesis can be applied to both prokaryotic and eukaryotic expression since the formation of polymer will not be dependent on specialized feedstocks. Thus, once a recombinant PHA monomer synthase is introduced into a prokaryotic or eukaryotic system, and co-expressed with the appropriate PHA synthase, novel bipolymer formation can occur.

### Example 3

### Cloning and Sequencing of the Vep ORFI PKS Gene Cluster

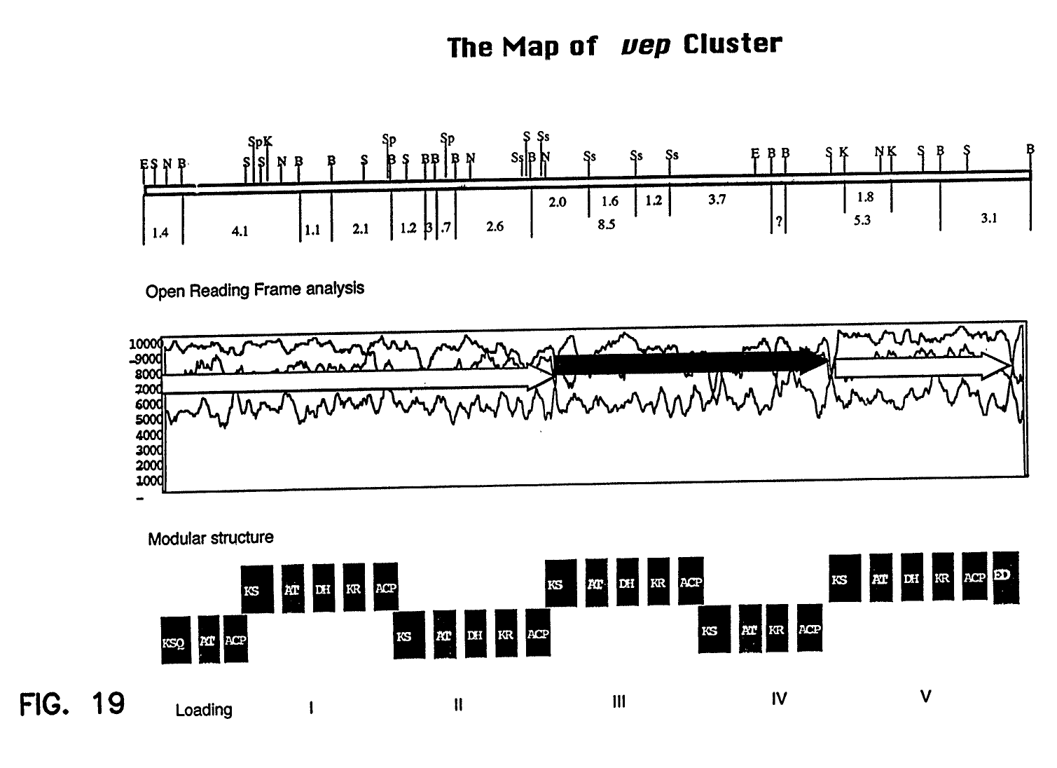

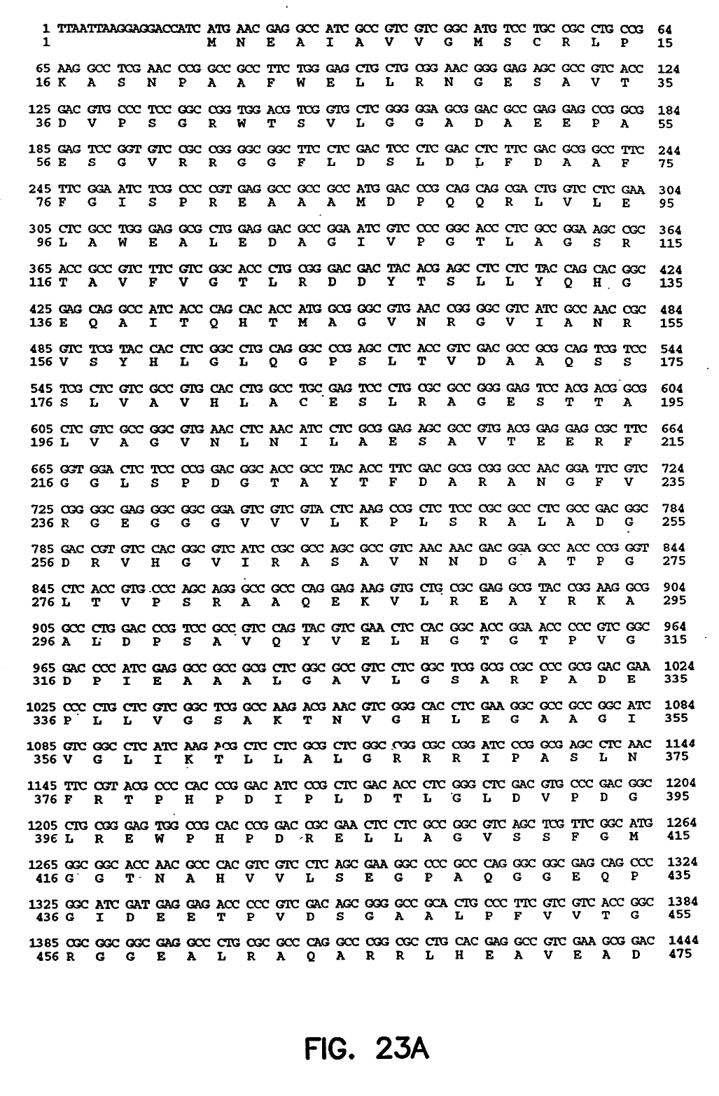

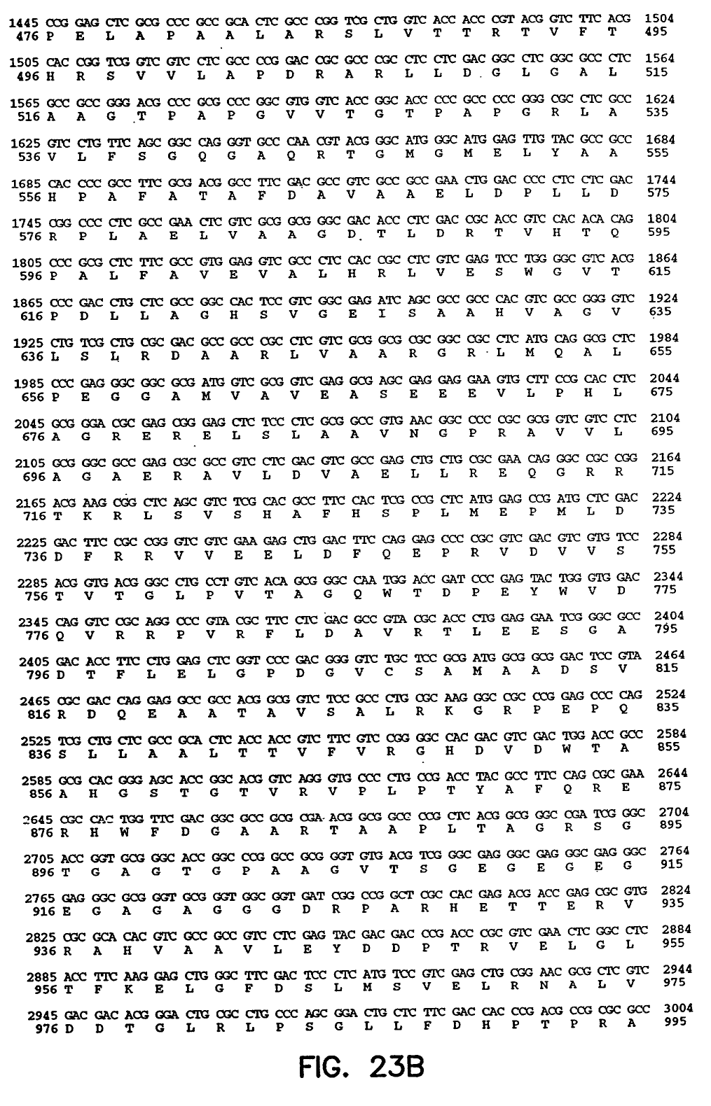

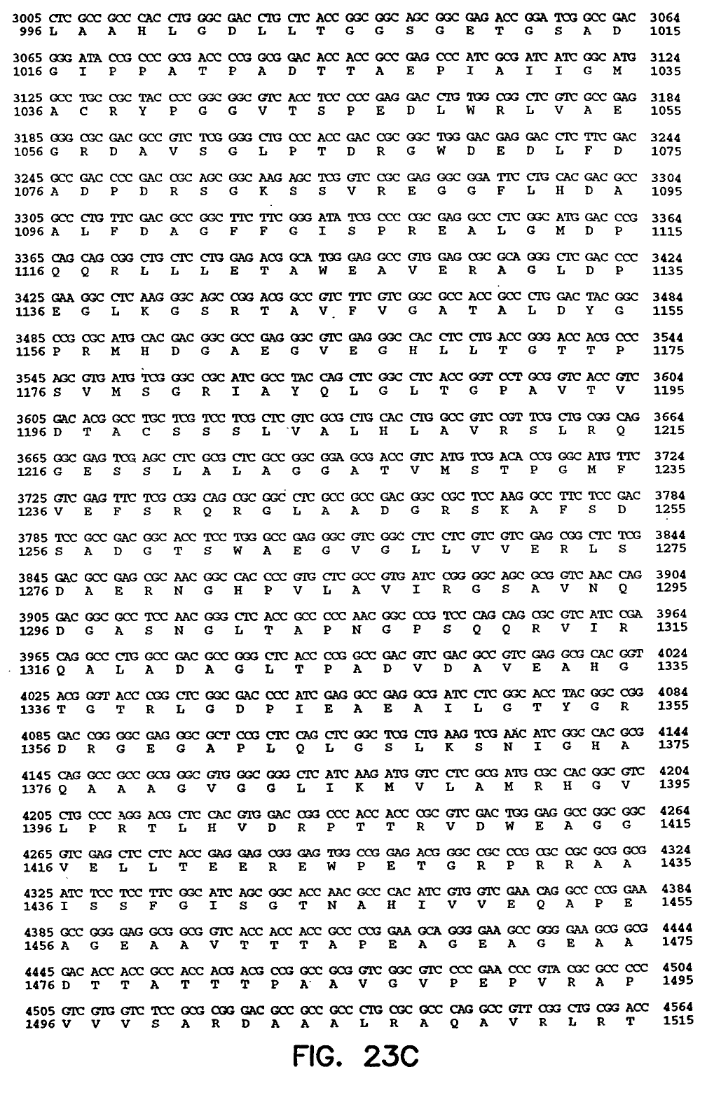

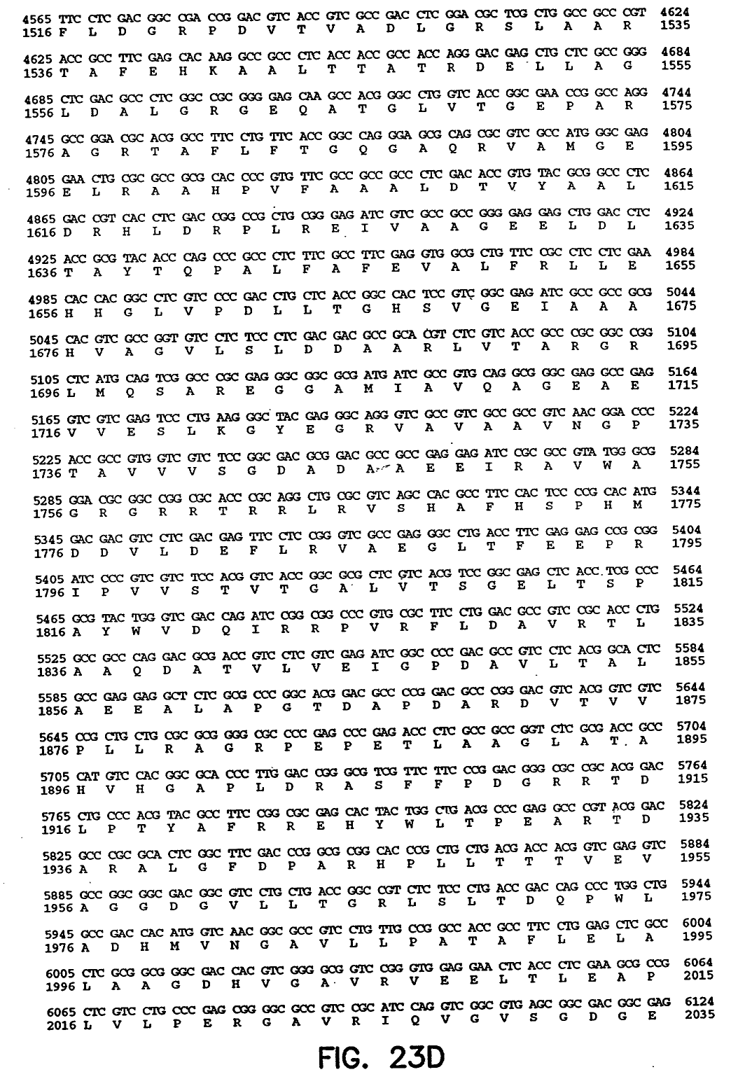

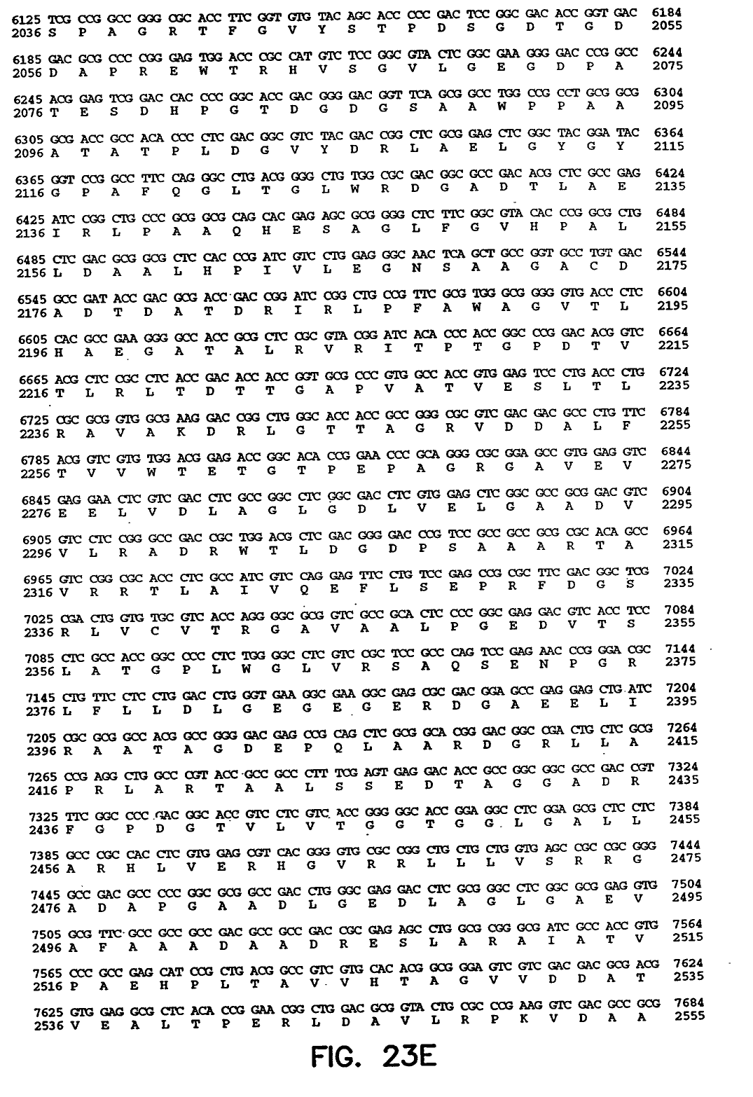

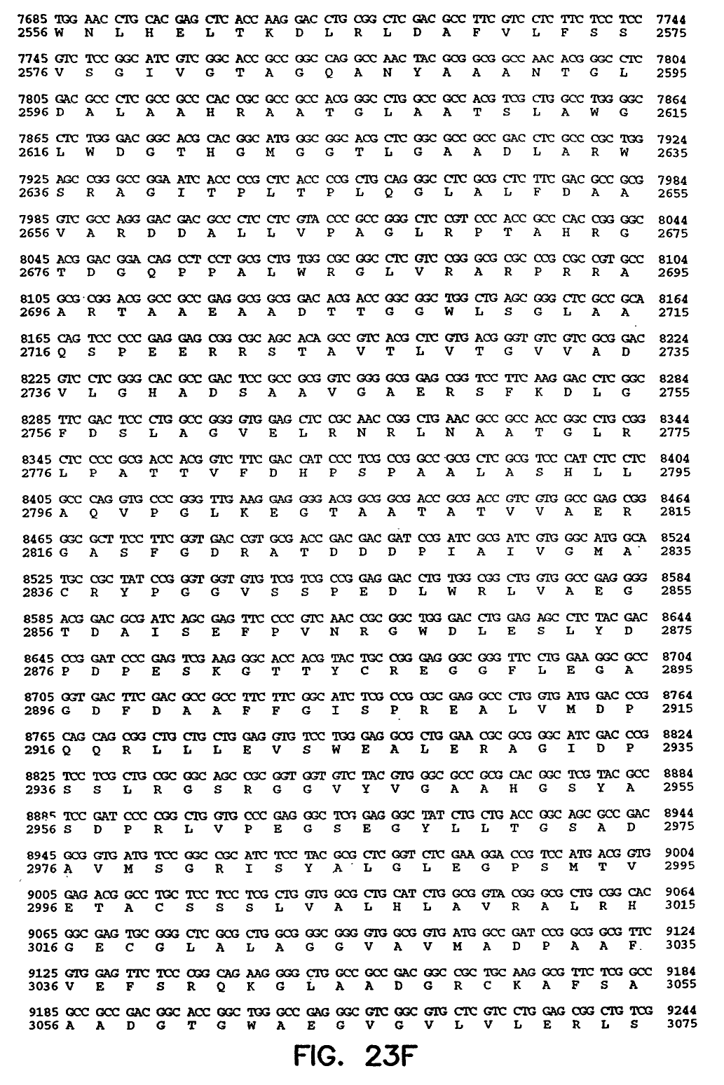

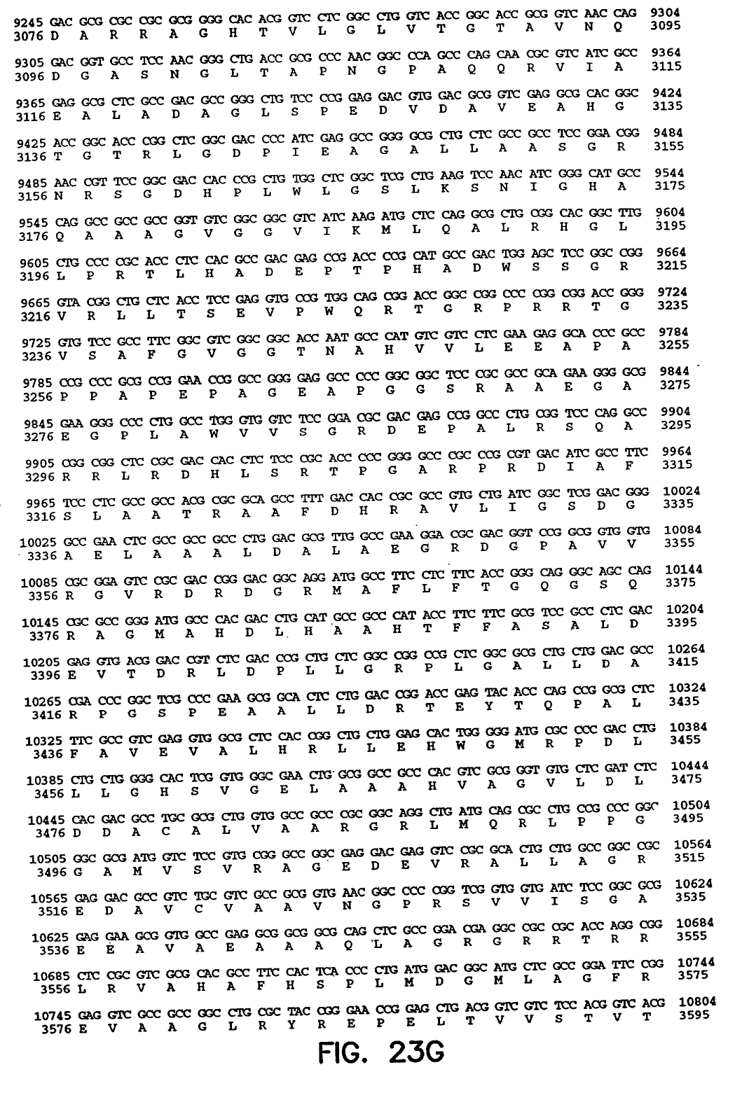

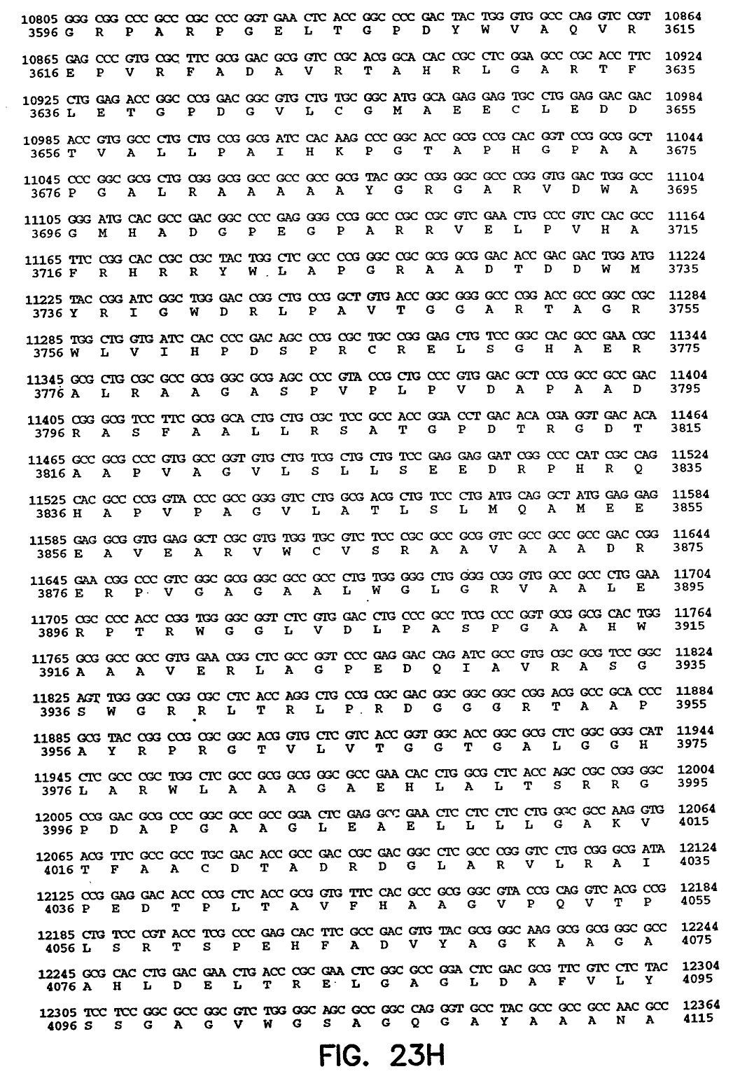

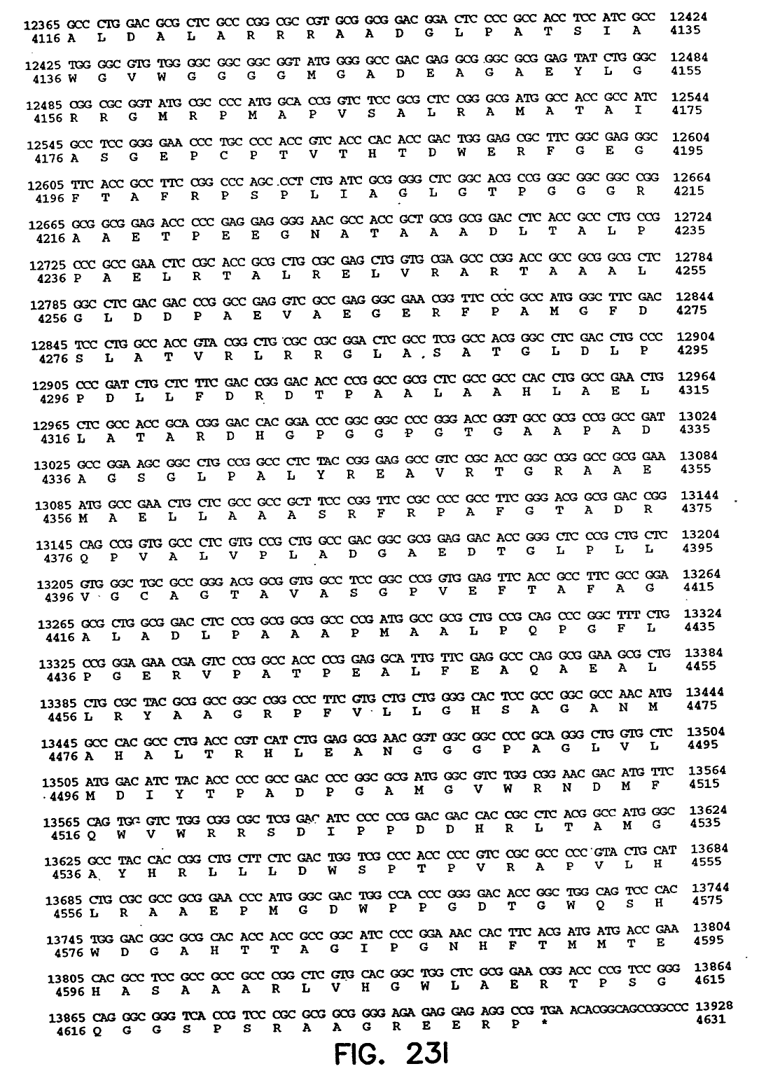

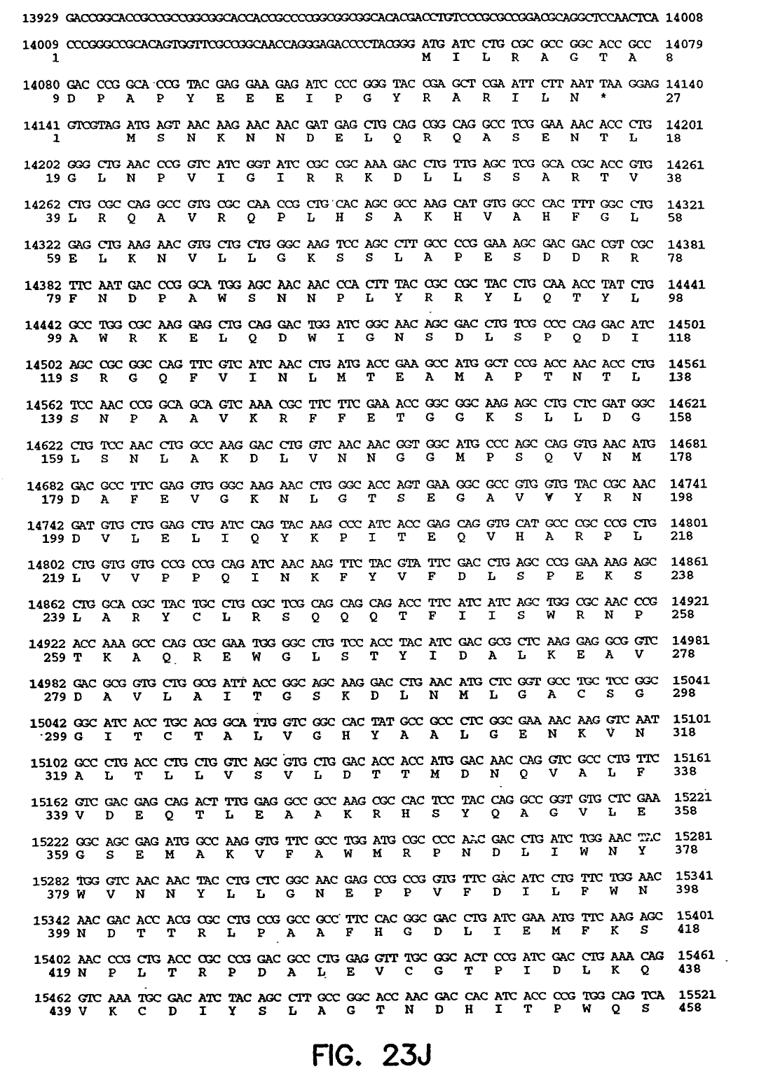

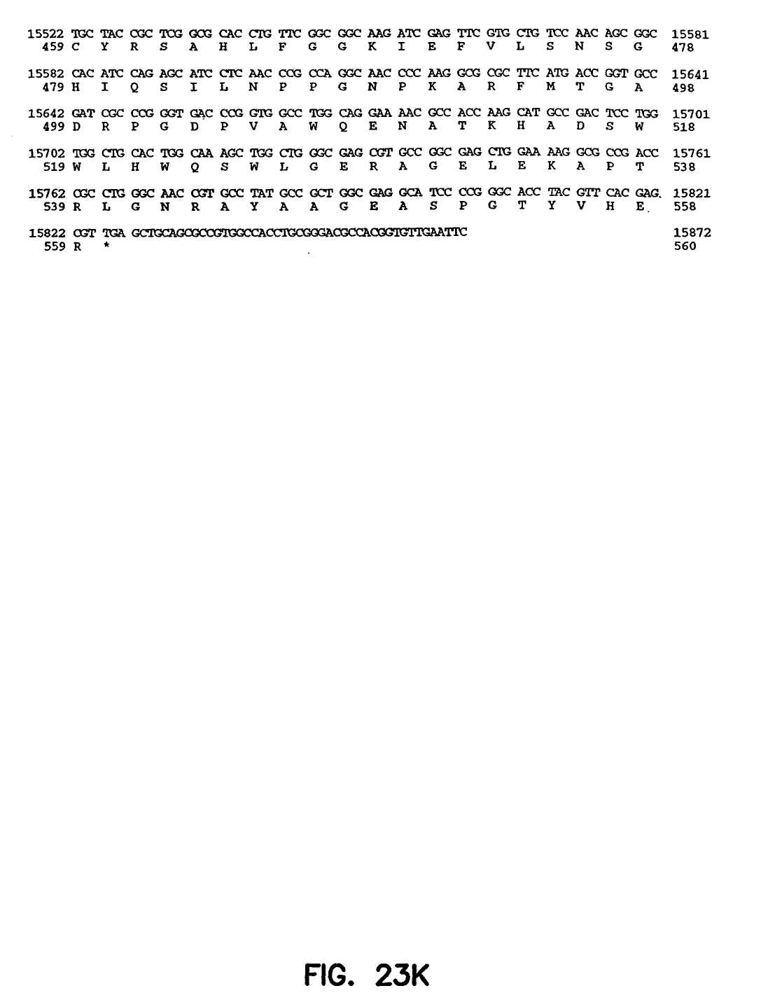

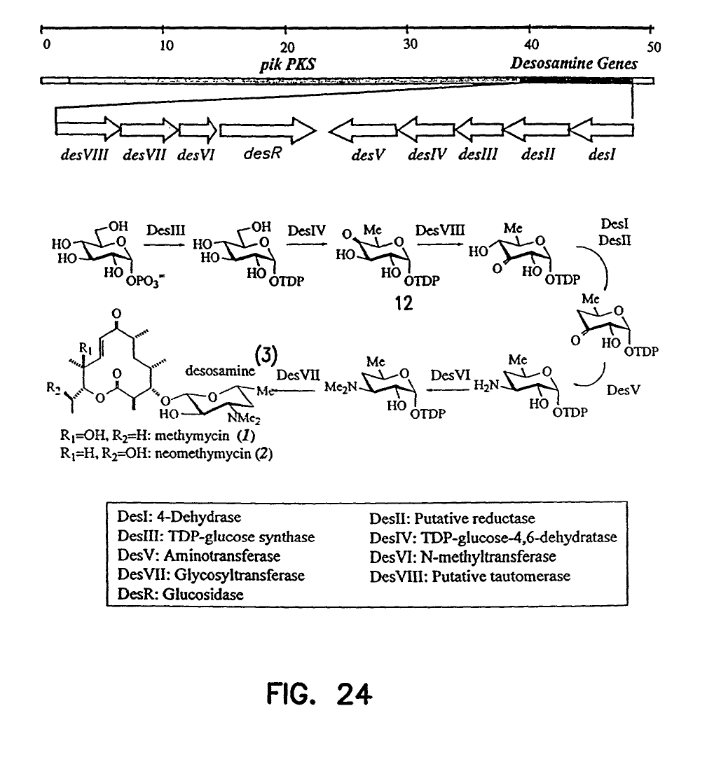

The entire PKS cluster form Streptomyces venezuelae was cloned using a heterologous hybridization strategy. A 1.2 kb DNA fragment that hybridized strongly to a DNA encoding an eryA PKS β-ketoacyl synthase domain was cloned and used to generate a plasmid for gene disruption. This method generated a mutant strain blocked in the synthesis of the antibiotic. A S. venezuelae genomic DNA library was generated and used to clone a cosmid containing the complete methymycin aglycone PKS DNA. Fine-mapping analysis was performed to identify the order and sequence of catalytic domains along the multifunctional PKS (FIG. 19). DNA sequence analysis of the vep ORFI showed that the order of catalytic domains is KS^{Q}/AT/ACP/KS/AT/KR/ACP/KS/AT/DH/KR/ACP. The complete DNA sequence, and corresponding amino acid sequence, of the vep ORFI is shown in FIG. 23 (SEQ ID NO:1 and SEQ ID NO:2, respectively).

The sequence data indicated that the PKS gene cluster encodes a polyene of twelve carbons. The vep gene cluster contains 5 polyketide synthase modules, with a loading module at its 5′ end and an ending domain at its 3′ end. Each of the sequenced modules includes a keto-ACP (KS), an acyltransferase (AT), a dehydratase (DH), a keto-reductase (KR), and an acyl carrier protein domain. The six acyltransferase domains in the cluster are responsible for the incorporation of six acetyl-CoA moieties into the product. The loading module contains a KSQ, an AT and an ACP domain. KSQ refers to a domain that is homologous to a KS domain except that the active site cysteine (C) is replaced by glutamine (Q). There is no counterpart to the KSQ domain in the PKS clusters which have been previously characterized.

The ending domain (ED) is an enzyme which is responsible for the attachment of the nascent polyketide chain onto another molecule. The amino acid sequence of ED resembles an enzyme, HetM, which is involved in Anabaena heterocyst formation. The homology between vep and HetM suggests that the polypeptide encoded by the vep gene cluster may synthesize a polyene-containing composition which is present in the spore coat or cell wall of its natural host, S. venezuelae.

### Example 4

### Preparation of a Vector Encoding a Saturated β-hydroxyhexanoyl CoA Monomer or an Unsaturated β-hydroxyhexanoyl CoA Monomer

To provide a recombinant monomer synthase that generates a saturated β-hydroxyhexanoylCoA or unsaturated β-hydroxyhexanoylCoA monomer, the linear correspondence between the genetic organization of the Type I macrolide PKS and the catalytic domain organization in the multifunctional proteins is assessed (Donadio et al., supra, 1991; Katz et al., Ann. Rev. Microbiol., 47, 875 (1993)). First, a DNA encoding a TE is added to the 3′ end of an ORFI of a Type I PKS, preferably the met ORFI (FIG. 6) as recently described by Cortes et al. (Science, 268, 1487 (1995)) in the erythromycin system. To ensure that the DNA encoding the TE is completely active, DNA encoding a linker region separating a normal ACP-TE region in a PKS, for example, the one found in met PKS ORF5 (FIG. 5), will be incorporated into the DNA. The resulting vector can be introduced into a host cell and the TE activity, rate of release of the CoA product, and identity of the fatty acid chain determined.

The acyl chain that is most likely to be released is the CoA ester, specifically the 3-hydroxy-4-methyl heptenoylCoA ester, since the filly elongated chain is presumably released in this form prior to macrolide cyclization. If the CoA form of the acyl chain is not observed, then a gene encoding a CoA ligase will be cloned and co-expressed in the host cell to catalyze formation of the desired intermediate.

There is clear precedent for release of the predicted premature termination products from mutant strains of macrolide-producing Streptomyces that produce intermediates in macrolide synthesis (Huber et al., Antimicrob. Agents Chemother., 34, 1535 (1990); Kinoshita et al., J. Chem. Soc., Chem. Comm., 14, 943 (1988)). The structure of these intermediates is consistent with the linear organization of functional domains in macrolide PKSs, particularly those related to eryA, tyl, and met. Other known PKS gene clusters include, but are not limited to, the gene cluster encoding 6-methylsalicylic acid synthase (Beck et al., Eur. J. Biochem., 192, 487 (1990)), soraphen A (Schupp et al., J. Bacteriol., 177, 3673 (1995)), and sterigmatocystin (Yu et al., J. Bacteriol., 177, 4792 (1995)).

Once the release of the 3-hydroxy-4-methyl heptenoylCoA ester is established, DNA encoding the extender unit AT in met module 1 is replaced to change the specificity from methylnalonylCoA to malonylCoA (FIGS. 4-6). This change eliminates methyl group branching in the β-hydroxy acyl chain. While comparison of known AT amino acid sequences shows high overall amino acid sequence conservation, distinct regions are readily apparent where significant deletions or insertions have occurred. For example, comparison of malonyl and methylmalonyl amino acid sequences reveals a 37 amino acid deletion in the central region of the malonyltransferase. Thus, to change the specificity of the methylmalonyl transferase to malonyl transferase, the met ORFI DNA encoding the 37 amino acid sequence of MMT will be deleted, and the resulting gene will be tested in a host cell for production of the desmethyl species, 3-hydroxyheptenoylCoA. Alternatively, the DNA encoding the entire MMT can be replaced with a DNA encoding an intact MT to affect the desired chain construction.

After replacing MMT with MT, DNA encoding DH/ER will be introduced into DNA encoding met ORFI module 1. This modification results in a multifunctional protein that generates a methylene group at C-3 of the acyl chain (FIG. 6). The DNA encoding DH/ER will be PCR amplified from the available eryA or tyl PKS sequences, including the DNA encoding the required linker regions, employing a primer pair to conserved sequences 5′ and 3′ of the DNA encoding DH/ER. The PCR fragment will then be cloned into the met ORFI. The result is a DNA encoding a multifunctional protein (MT* DH/ER*TE*). This protein possesses the full complement of keto group processing steps and results in the production of heptenoylCoA.

The DNA encoding dehydrase in met module 2 is then inactivated, using site-directed mutagenesis in a scheme similar to that used to generate the rat FAS DH- described above (Joshi et al., J. Biol. Chem., 268, 22508 (1993)). This preserves the required (R)-3-hydroxy group which serves as the substrate for PHA synthases and results in (R)-3-hydroxyheptanoylCoA species.

The final domain replacement will involve the DNA encoding the starter unit acyltransferase in met module 1 (FIG. 5), to change the specificity from propionyl CoA to acetyl CoA. This shortens the (R)-3-hydroxy acyl chain from heptanoyl to hexanoyl. The DNA encoding the catalytic domain will need to be generated based on a FAS or 6-methylsalicylic acid synthase model (Beck et al., Eur,. J. Biochem., 192, 487 (1990)) or by using site-directed mutagenesis to alter the specificity of the resident met PKS propionyltransferase sequence. Limiting the initiator species to acetylCoA can result in the use of this starter unit by the monomer synthase. Previous work with macrolide synthases have shown that some are able to accept a wide range of starter unit carboxylic acids. This is particularly well documented for avermectin synthase, where over 60 new compounds have been produced by altering the starter unit substrate in precursor feeding studies (Dutton et al., J. Antibiotics, 44, 357 (1991)).

### Example 5

### Preparation of a Vector Encoding a Recombinant Monomer Synthase that Synthesizes 3-hydroxy-4-hexenoic Acid

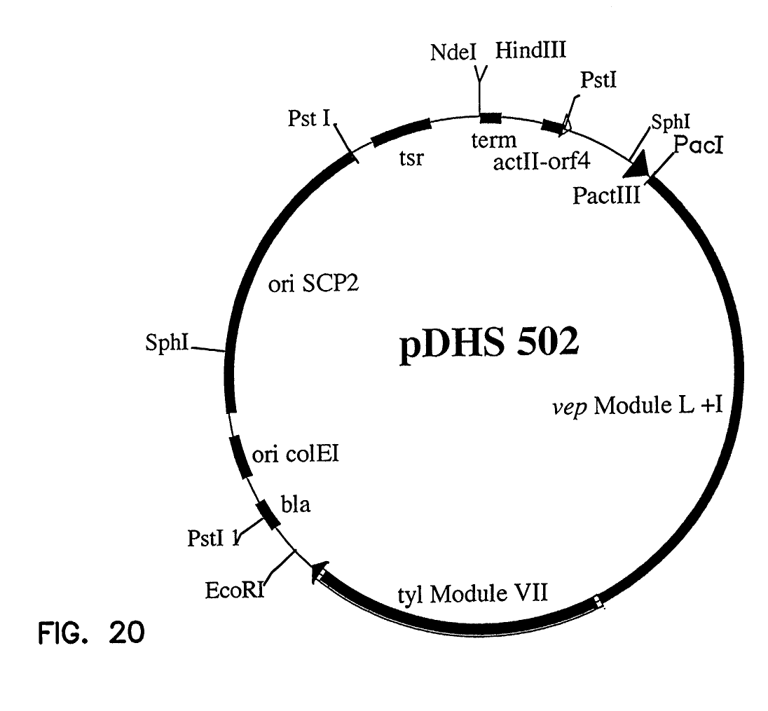

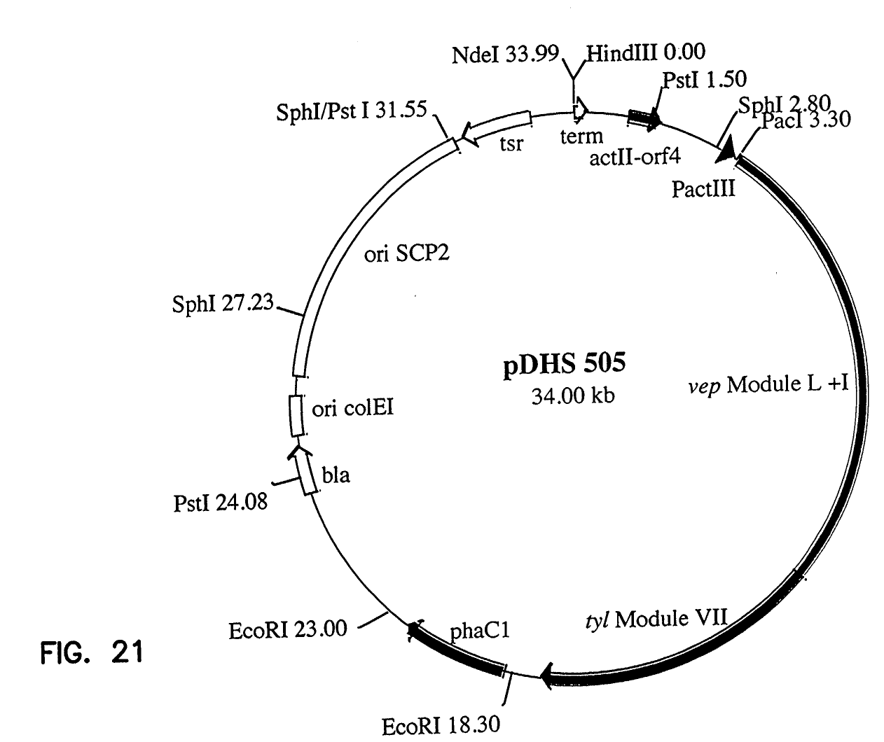

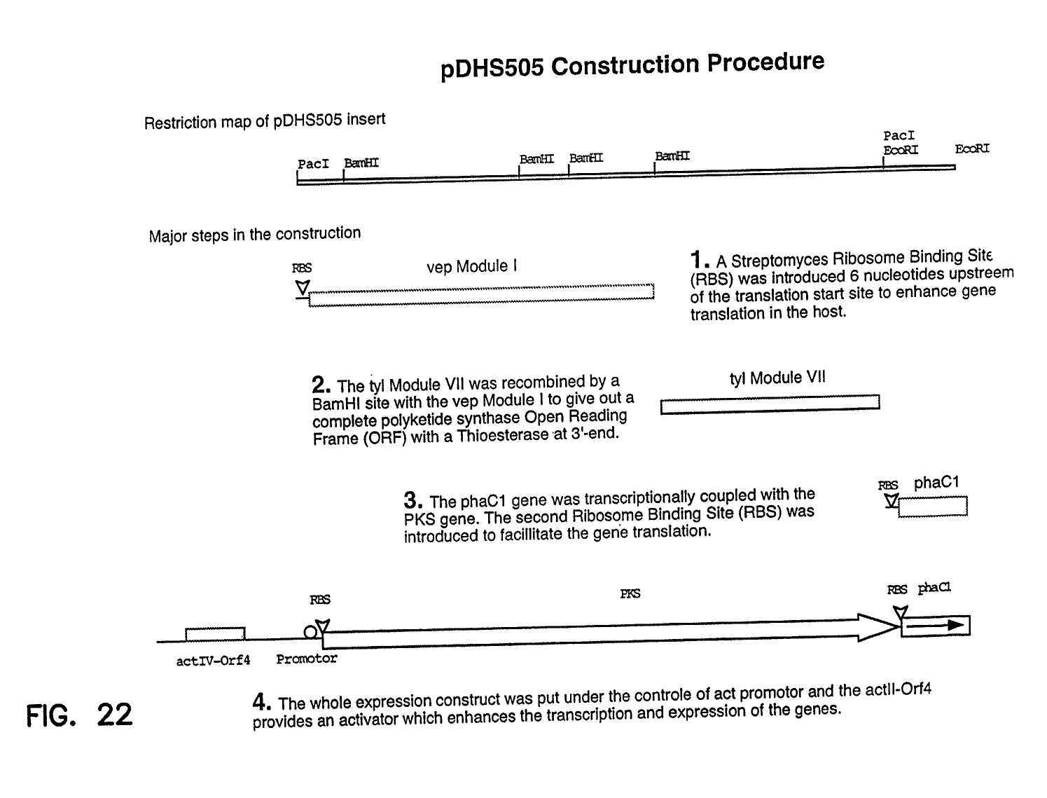

To provide a recombinant monomer synthase that synthesizes 3-hydroxyl-4-hexenoic acid, a precursor for polyhydroxyhexenoate, the DNA segment encoding the loading and the first module of the vep gene cluster was linked to the DNA segment encoding module 7 of the tyl gene cluster so as to yield a recombinant DNA molecule encoding a fusion polypeptide which has no amino acid differences relative to the corresponding amino acid sequence of the parent modules. The fusion polypeptide catalyzes the synthesis of 3-hydroxyl-4-hexenoic acid. The recombinant DNA molecule was introduced into SCP2, a Streptomyces vector, under the control of the act promoter (pDHS502, FIG. 20). A polyhydroxyalkanoate polymerase gene, phacI from Pseudomonas oleavorans, was then introduced downstream of the recombinant PKS cluster (pDHS505; FIGS. 22 and 23). The DNA segment encoding the polyhydroxyalkanoate polymerase is linked to the DNA segment encoding the recombinant PKS synthase so as to yield a fusion polypeptide which synthesizes polyhydroxyhexenoate in Streptomyces. Polyhydroxyhexenoate, a biodegradable thermoplastic, is not naturally synthesized in Streptomyces, or as a major product in any other organism. Moreover, the unsaturated double bond in the side chain of polyhydroxyhexenoate may result in a polymer which has superior physical properties as a biodegradable thermoplastic over the known polyhydroxyalkanoates.

### Example 6

### Deletion of the desR Gene of the Desosamine Biosynthetic Gene Cluster

As some macrolides have more than one attached sugar moiety, the assignment of sugar biosynthetic genes to the appropriate sugar biosynthetic pathway can be quite difficult. Since methymycin (a compound of formula (1)) and neomethymycin (a compound of formula (2)) (FIG. 24) (Donin et al., 1953; Djerassi et al., 1956), two closely related macrolide antibiotics produced by Streptomyces venezuelae, contain desosamine as their sole sugar component, the organization of the sugar biosynthetic genes in the methymycin/neomethymycin gene cluster may be less complicated. Thus, this system was chosen for the study of the biosynthesis of desosamine, a NN-dimethylamino-3,4,6-trideoxyhexose, which also exists in the erythromycin structure (Flinn et al., 1954).

To study the formation of this unusual sugar, a DNA library was constructed by partially digesting the genomic DNA of S. venezuelae (ATCC 15439) with Sau3A I into 35-40 kb fragments which were ligated into the cosmid vector pNJ1 (Tuan et al., 1990). The recombinant DNA was packaged into bacteriophage λ which was used to transfect E. coli DH5α. The resulting cosmid library was screened for desired clones using the tylA1 and tylA2 genes from the tylosin biosynthetic cluster as probes (Baltz et al., 1988; Merson-Davies et al., 1994). These two probes are specific for sugar biosynthetic genes whose products catalyze the first two steps universally followed by all unusual 6-deoxyhexoses studied thus far. The initial reaction involves conversion of glucose-l-phosphate to TDP-D-glucose by α-D-glucose-1-phosphate thymidylyltransferase (TylA1) and subsequently, TDP-D-glucose is transformed to TDP-4-keto-6-deoxy-D-glucose by TDP-D-glucose 4,6-dehydratase (TylA2). Three cosmids were found to contain genes homologous to tylA1 and tylA2. Further analysis of these cosmids led to the identification of nine open reading frames (ORFs) downstream of the PKS genes (FIG. 24). Based on sequence similarities to other sugar biosynthetic genes, especially those derived form the erythromycin cluster (Gaisser et al., 1997; Summers et al., 1997), eight of these nine ORFs are believed to be involved in the biosynthesis of TDP-D-desosamine. Interestingly, the ery cluster lacks homologs of the tylA1 and tylA2 genes that are responsible for the first two steps in desosamine pathway. It is possible that the erythromycin biosynthetic machinery may rely on a general cellular pool of TDP-4-keto-6-deoxy-D-glucose for mycarose and desosamine formation. Depicted in FIG. 24 is a biosynthetic pathway for TDP-D-desosamine.

Although eight of the nine ORFs have been assigned to desosamine formation, the presence of desR, which shows strong sequence homology to β-glucosidases (as high as 39% identity and 46% similarity) (Castle et al., 1998), within the desosamine gene cluster is puzzling. To investigate the function of DesR relative to the biosynthesis of methymycin/neomethymycin, a disruption plasmid (pBL1005) derived from pKC1139 (containing an apramycin resistance marker) (Bierman et al., 1992) was constructed in which a 1.0 kb NcoI/XhoI fragment of the desR gene was deleted and replaced by the thiostrepton Iresistance (tsr) gene (1.1 kb) (Bibb et al., 1985) via blunt-end ligation. This plasmid was used to transform E. coli S17-1, which serves as the donor strain to introduce the pBL1005 construct through conjugal transfer into the wild-type S. venezuelae (Biertnan et al., 1992). The double crossover mutants in which chromosomal desR had been replaced with the disrupted gene were selected according to their thiostrepton-resistant and apramycin-sensitive characteristics. Southern blot hybridization analysis was used to confirm the gene replacement.

The desired mutant was first grown at 29° C. in seed medium for 48 hours, and then inoculated and grown in vegetative medium for another 48 hours (Cane et al., 1993). After the fermentation broth was centrifuiged at 10,000 g to remove cellular debris and mycelia, the supernatant was adjusted to pH 9.5 with concentrated KOH, and extracted with an equivolume of chloroform (four times). The organic layer was dried over sodium sulfate and evaporated to dryness. The amber oil-like crude products were first subjected to flash chromatography on silica gel using a gradient of 0-40% methanol in chloroform, followed by HPLC purification on a C₁₈ column eluted isocratically with 45% acetonitrile in 57 mM ammonium acetate (pH 6.7). In addition to methymycin (a compound of formula (1)) and neomethymycin (a compound of formula (2)), two new products were isolated. The yield of a compound of formula (13) and a compound of formula (14) was each in the range of 5-10 mg/L of fermentation broth. However, a compound of formula (1) and a compound of formula (2) remained to be the major products. High-resolution FAB-MS revealed that both compounds have identical molecular compositions that differ from methymycin/neomethymycin by an extra hexose. The chemical nature of these two new compounds were elucidated to be C-2′ β-glucosylated methymycin and neomethymycin (a compound of formula (13) and formula (14), respectively) by extensive spectral analysis.

The spectral data of (13): ¹H NMR (acetone-d₆) δ 6.56 (1H, d, J=16.0, 9-H), 6.46 (1H, d, J=16.0, 8-H), 4.67 (1H, dd, J=10.8, 2.0, 11-H), 4.39 (1H, d, J=7.5, 1′H), 4.32 (1H, d, J=8.0, 1″-H), 3.99 (1H, dd, J=11.5, 2.5, 6″-H), 3.72 (1H, dd, J=11.5, 5.5, 6″-H), 3.56 (1H, m, 5′-H), 3.52 (1H, d, J=10.0, 3-H), 3.37 (1H, t, J=8.5, 3″-H), 3.33 (1H, m, 5″-H), 3.28 (1H, t, J=8.5, 4″-H), 3.23 (1H, dd, J=10.5, 7.5, 2′-H), 3.15 (1H, dd, J=8.5, 8.0, 2″-H), 3.10 (1H, m, 2-H), 2.75 (1H, 3′-H, buried under H₂O peak), 2.42 (1H, m, 6-H), 2.28 (6H, s, NMe₂), 1.95 (1H, m, 12-H), 1.9 (1H, m, 5-H), 1.82 (1H, m, 4′-H), 1.50 (1H, m, 12-H), 1.44 (3H, d, J=7.0, 2-Me), 1.4 (1H, m, 5-H), 1.34 (3H, s, 10-Me), 1.3 (1H, m, 4-H), 1.25 (1H, m, 4′-H), 1.20 (3H, d, J=6.0, 5′-Me), 1.15 (3H, d, J=7.0, 6-Me), 0.95 (3H, d, J=6.0, 4-Me), 0.86 (3H, t, J=7.5, 12-Me). High-resolution FAB-MS: calc for C₃₁H₅₄NO₁₂ (M+H)⁺632.3646, found 632.3686.

Spectral data of (14): ¹H NMR (acetone-d₆) δ 6.69 (1H, dd, J=16.0, 5.5 Hz, 9-H), 6.55 (1H, dd, J=16.0, 1.3, 8-H), 4.71 (1H, dd, J=9.0, 2.0, 11-H), 4.37 (1H, d, J=7.0, 1′-H), 4.31 (1H, d, J=8.0, 1″-H), 3.97 (1H, dd, J=11.5, 2.5, 6″-H), 3.81 (1H, dq, J=9.0, 6.0, 12-H), 3.72 (1H, dd, J=11.5, 5.0, 6″-H), 3.56 (1H, m, 5′-H), 3.50 (1H, bd, J=10.0, 3-H), 3.36 (1H, t, J=8.5, 3″-H), 3.32 (1H, m, 5″-H), 3.30 (1H, t, J=8.5, 4″-H), 3.23 (1-H, dd, J=10.2, 7.0, 2′-H), 3.13, (1H, dd, J=8.5, 8.0, 2″-H), 3.09 (1H, m, 2-H), 3.08 (1H, m, 10-H), 2.77 (1H, ddd, J=12.5, 10.2, 4.5, 3′-H), 2.41 (1H, m, 6-H), 2.28 (6H, s, NMe₂), 1.89 (1H, t, J=13.0, 5-H), 1.83 (1H, ddd, J=12.5, 4.5, 1.5, 4′-H), 1.41 (3H, d, J=7.0, 2-Me), 1.3 (1H, m, 4-H), 1.25 (1H, m, 5-H), 1.2 (1H, m 4′-H, 1.20 (3H, d, J=6.0 5′-Me), 1.17 (6H, d, J=7.0, 6-Me, 10-Me), 1.12 (3H, d, J=6.0, 12-me), 0.96 (3H, d, J=6.0, 4-Me). ¹³C NMR (acetone-d₆) δ 204.1 (C-7), 175.8 (C-1), 148.2 (C-9), 126.7 (C-8), 108.3 (C-1″), 104.2 (C-1′) 85.1 (C-3), 83.0 (C-2′), 78.2 (C-3″), 78.1 (C-5″), 76.6 (C-2″), 76.4 (C-11), 71.8 (C-4″), 69.3 (C-5′), 66.1 (C-12), 66.0 (C-3′), 63.7 (C-6″), 46.2 (C-6), 44.4 (C-2), 40.8 (NMe₂), 36.4 (C-10), 34.7 (C-5), 34.0 (C-4), 29.5 (C-4′), 21.5 (5′-Me), 21.5 (12-Me), 17.9 (6-Me), 17.7 (4-Me), 17.2 (2-Me), 9.9 (10-Me). High-resolution FAB-MS: calc for C₃₁H₅₄NO₁₂ (M+H)⁺632.3646, found 632.3648.

The coupling constant (d, J=8.0 Hz) of the anomeric hydrogen (1″-H) of the added glucose and the magnitude of the downfield shift (11.8 ppm) of C-2′of desosamine are all consistent with the assigned C-2′β-configuration (Seo et al., 1978).

The antibiotic activity of a compound of formula (13) and (14) against Streptococcus pyogenes was examined by separately applying 20 μL of each sample (1.6 mM in MeOH) to sterilized filter paper discs which were placed onto the surface of S. pyogenes grown on Mueller-Hinton agar plates (Mangahas, 1996). After being grown overnight at 37° C., the plates of the controls (a compound of formula (1) and (2)) showed clearly visible inhibition zones. In contrast, no such clearings were discernible around the discs of a compound of formula (13) and (14). Evidently, β-glucosylation at C-2′of desosamine in methymycin/neomethymycin renders these antibiotics inactive.

It should be noted that similar phenomena involving inactivation of macrolide antibiotics by glycosylation are known (Celmer et al., 1985; Kuo et al., 1989; Sasaki et al., 1996). For example, it was found that when erythromycin was given to Streptomyces lividans, which contains a macrolide glycosyltransferase (MgtA), the bacterium was able to defend itself by glycosylating the drug (Cundliffe, 1992; Jenkins et al., 1991). Such a macrolide glycosyltransferase activity has been detected in 15 out of a total of 32 actinomycete strains producing various polyketide antibiotics (Sasaki et al., 1996). Interestingly, the co-existence of a macrolide glycosyltransferase (OleD) capable of deactivating oleandomycin by glucosylation (Hernandez et al., 1993), and an extracellular β-glucosidase capable of removing the added glucose from the deactivated oleandomycin in Streptomyces antibioticus (Vilches et al., 1992) has led to the speculation of glycosylation as a possible self-resistance mechanism in S. antibioticus. Although the genes of the aforementioned glycosyltransferases have been cloned in a few cases, such as mgtA of S. lividans and oleD of S. antibioticus, the whereabouts of macrolide β-glycosidase genes remain obscure. Interestingly, the recently released eryBI sequence, which is part of the erythromycin biosynthetic cluster, is highly homologous to desR (55% identity) (Gaisser et al., 1997).

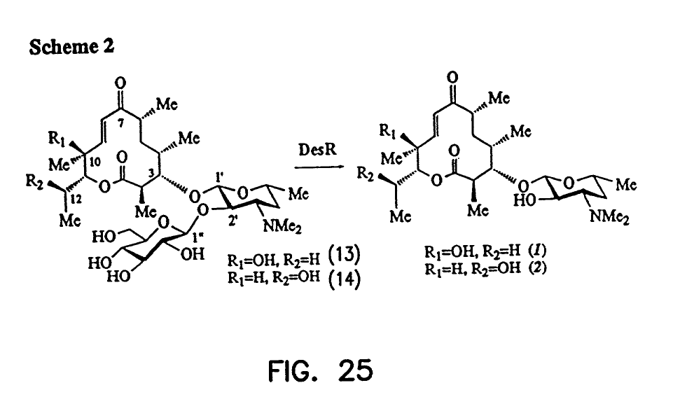

The discovery of desR, a macrolide β-glucosidase gene, within the desosamine gene cluster is thus significant, and the accumulation of deactivated compounds of formula (13) and (14) after desR disruption provides direct molecular evidence indicating that a similar self-defense mechanism via glycosylation/deglycosylation may also be operative in S. venezuelae. However, because a significant amount of methymycin and neomethymycin also exist in the fermentation broth of the mutant strain, glucosylation of desosamine may not be the primary self-resistance mechanism in S. venezuelae. Indeed, an rRNA methyltransferase gene found upstream from the PKS genes in this cluster may confer the primary self-resistance protection. Thus, these results are consistent with the fact that antibiotic producing organisms generally have more than one defensive option (Cundliffe, 1989). In light of this observation, it is conceivable that methymycin/neomethymycin may be produced in part as the inert diglycosides (a compound of formula (13) or (14)), and the macrolide β-glucosidase encoded by desR is responsible for transforming methymycin/neomethymycin from their dormant state to their active form. Supporting this idea, the translated desR gene has a leader sequence characteristic of secretory proteins (von Heijne, 1986; von Heijne, 1989). Thus, DesR may be transported through the cell membrane and hydrolyze the modified antibiotics extracellularly to activate them (FIG. 25).

Summary

Inspired by the complex assembly and the enzymology of aminodeoxy sugars that are frequently found as essential components of macrolide antibiotics, the entire desosamine biosynthetic gene cluster from the methymycin and neomethymycin producing strain Streptomyces venezuelae was cloned, sequenced, and mapped. Eight of the nine mapped genes were assigned to the biosynthesis of TDP-D-desosamine based on sequence similarities to those derived from the erythromycin cluster. The remaining gene, designated desR, showed strong sequence homology to β-glucosidases.

To investigate the function of the encoded protein (DesR), a disruption mutant was constructed in which a NcoI/XhoI fragment of the desR gene was deleted and replaced by the thiostrepton resistance (tsr) gene. In addition to methymycin and neomethymycin, two new products were isolated from the fermentation of the mutant strain. These two new compounds, which are biologically inactive, were found to be C-2′ β-glucosylated methymycin and neomethymycin. Since the translated desR gene has a leader sequence characteristic of secretory proteins, the DesR protein may be an extracellular β-glucosidase capable of removing the added glucose from the modified antibiotics to activate them. Thus, the occurrence of desR within the desosamine gene cluster and the accumulation of deactivated glucosylated methymycin/neomethymycin upon disruption of desR provide strong molecular evidence suggesting that a self-resistance mechanism via glucosylation may be operative in S. venezuelae.

Thus, the desR gene can be used as a probe to identify homologs in other antibiotic biosynthetic pathways. Deletion of the corresponding macrolide glycosidase gene in other antibiotic biosynthetic pathways may lead to the accumulation of the glycosylated products which may be used as prodrugs with reduced cytotoxicity. Glycosylation also holds promise as a tool to regulate and/or minimize the potential toxicity associated with new macrolide antibiotics produced by genetically engineered microorganisms. Moreover, the availability of macrolide glycosidases, which can be used for the activation of newly formed antibiotics that have been deliberately deactivated by engineered glycosyltransferases, may be useful in the development of novel antibiotics using the combinatorial biosynthetic approach (Hopwood et al., 1990; Katz et al., 1993; Hutchinson et al., 1995; Carreras et al., 1997; Kramer et al., 1996; Khosla et al., 1996; Jacobsen et al., 1997; Marsden et al., 1998).

### Example 7

### Deletion of the desVI Gene of the Desosamine Biosynthetic Gene Cluster

The emergence of pathogenic bacteria resistant to many commonly used antibiotics poses a serious threat to human health and has been the impetus of the present resurgent search for new antimicrobial agents (Box et al., 1997; Davies, 1996; Service, 1995). Since the first report on using genetic engineering techniques to create “hybrid” polyketides (Hopwood et al., 1995), the potential of manipulating the genes governing the biosynthesis of secondary metabolites to create new bioactive compounds, especially macrolide antibiotics, has received much attention (Kramer et al., 1996; Khosla et al., 1996). This class of clinically important drugs consists of two essential structural components: a polyketide aglycone and the appended deoxy sugars (Omura, 1984). The aglycone is synthesized via sequential condensations of acyl thioesters catalyzed by a highly organized multi-enzyme complex, polyketide synthase (PKS) (Hopwood et al., 1990; Katz, 1993; Hutchinson et al., 1995; Carreras et al., 1997). Recent advances in the understanding of the polyketide biosynthesis have allowed recombination of the PKS genes to construct an impressive array of novel skeletons (Kramer et al., 1996; Khosla et al., 1996; Hopwood et al., 1990; Katz, 1993; Hutchinson et al., 1995; Carreras et al., 1997; Epp et al., 1989; Donadio et al., 1993; Arisawa et al., 1994; Jacobsen et al., 1997; Marsden et al., 1998). Without the sugar components, however, these new compounds are usually biologically impotent. Hence, if one plans to make new macrolide antibiotics by a combinatorial biosynthetic approach, two immediate challenges must be overcome: assembling a repertoire of novel sugar structures and then having the capacity to couple these sugars to the structurally diverse macrolide aglycones.

Unfortunately, knowledge of the formation of the unusual sugars in these antibiotics remains limited (Liu et al., 1994; Kirschning et al., 1997; Johnson et al., 1998). Part of the reason for this comes from the fact that the sugar genes are generally scattered at both ends of the PKS genes. Such an organization within the macrolide biosynthetic gene cluster makes it difficult to distinguish the sugar genes from those encoding regulatory proteins or aglycone modification enzymes that are also interspersed in the same regions. The task can be made even more formidable if the macrolides contain multiple sugar components. In view of the “scattered” nature of the sugar biosynthetic genes, the antibiotic methymycin (a compound of formula (1) in FIG. 24) and its co-metabolite, neomethymycin (a compound of formula (2) in FIG. 24)), of Streptomyces venezuelae present themselves as an attractive system to study the formation of deoxy sugars (Donin et al., 1953; Djerassi et al., 1956). First, they carry D-desosanime (a compound of formula (3)) a prototypical aminodeoxy sugar that also exists in erythromycin. Second, since desosamine is the only sugar attached to the macrolactone of formula (1) and (2), identification of the sugar biosynthetic genes within the methymycin/neomethymycin gene cluster should be possible with much more certainty.

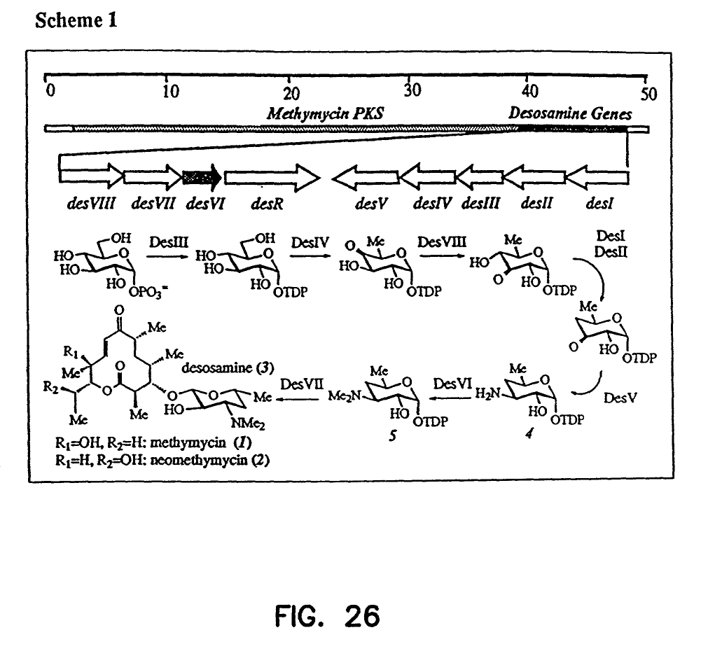

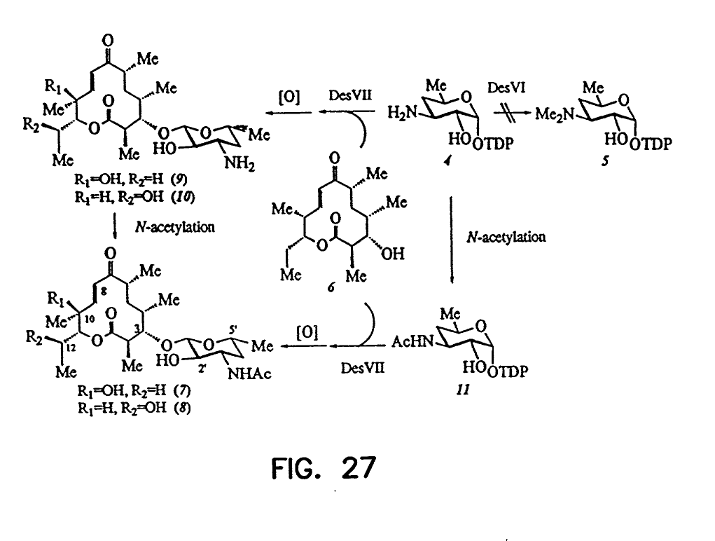

A 10 kb stretch of DNA downstream from the methymycin/neomethymycin gene cluster, which is about 60 kb in length, was found to harbor the entire desosamine biosynthetic gene cluster (FIG. 26). Among the nine open reading frames (ORFs) mapped in this segment, eight are likely to be involved in desosamine formation, while the remaining one, desR, encodes a macrolide β-glycosidase that may be involved in a self-resistance mechanism. Their identities, shown in FIG. 26, are assigned based on sequence similarities to other sugar biosynthetic genes (Gaisser et al., 1997; Summers et al., 1997). The proposed pathway is well founded on literature precedent and mechanistic intuition for the construction of aminodeoxy sugars (Liu et al., 1994; Kirschning et al., 1997; Johnson et al., 1998).

To determine whether new methymycin/neomethymycin analogues carrying modified sugars could be generated by altering the desosamine biosynthetic genes, the desVI gene, which has been predicted to encode the N-methyltransferase, was chosen as a target (Gaisser et al., 1997; Summers et al., 1997). The deduced desVI product is most closely related to that of eryCVI from the erythromycin producing strain Saccharopolyspora erythraea (70% identity), and also strongly resembles the predicted products of rdmD from the rhodomycin cluster of Streptomyces purpurascens (Niemi et al., 1995), srmX from the spiromycin cluster of Streptomyces ambofaciens (Geistlich et al., 1992), and tylM1 from the tylosin cluster of Streptomyces fradiae (Gandecha et al., 1997). All of these enzymes contain the consensus sequence LLDV(I)ACGTG (SEQ ID NO:25) (Gaisser et al., 1997; Summers et al., 1997), near their N-terminus, which is part of the S-adenosylmethionine binding site (Ingrosso et al., 1989; Haydock et al., 1991).

The deletion of desVI should have little polar effect (Lin et al., 1984) on the expression of other desosamine biosynthetic genes because the ORF (desR) lying immediately downstream from desVI is not directly involved in desosamine formation, and those lying further downstream are transcribed in the opposite direction. Second, since N,N-dimenthylation is almost certainly the last step in the desosamine biosynthetic pathway (Liu et al., 1994; Kirschning et al., 1997; Johnson et al., 1998; Gaisser et al., 1997; Summers et al., 1997), perturbing this step may lead to the accumulation of a compound of formula (4), which stands the best chance among all other intermediates of being recognized by the glycosyltransferase (DesVII) for successful linkage to the macrolactone of formula (6) (FIG. 25). Deletion and/or disruption of a single biosynthetic gene often affects the pathway at more than one specific step. In fact, disruption of eryCVI, the desVI equivalent in the erythromycin cluster, which has been predicted to encode a similar N-methylase to make desosamine in erythromycin (Gaisser et al., 1997; Summers et al., 1997), led to the accumulation of an intermediate devoid of the entire desosamine moiety (Summers et al., 1997).

A plasmid pBL3001, in which desVI was replaced by the thiostrepton gene (tsr) (Bibb et al., 1985), was constructed and introduced into wild type S. venezuelae by conjugal transfer using E. coli S17-1 (Bierman et al., 1992). Two identical double crossover mutants, KdesVI-21 and KdesVI-22 with phenotypes of thiostrepton resistance (Thio^{R}) and apamycin sensitivity (Apm^{S}) were obtained. Southern blot hybridization using tsr or a 1.1 kb HincII fragment from the desVII region further confirmed that the desVI gene was indeed replaced by tsr on the chromosome of these mutants. The KdesVI-21 mutant was first grown at 29° C. in seed medium (100 mL) for 48 hours, and then inoculated and grown in vegetative medium (3 L) for another 48 hours (Cane et al., 1993). The fermentation broth was centrifuged to remove the cellular debris and mycelia, and the supernatant was adjusted to pH 9.5 with concentrated KOH, followed by extraction with chloroform. No methymycin or neomethymycin was found; instead, the 10-deoxy-methynolide (6) (350 mg) (Lambalot et al., 1992) and two new macrolides containing an N-acetylated amino sugar, a compound of formula (7) (20 mg) and a compound of formula (8) (15 mg), were isolated. Their structures were determined by spectral analyses and high-resolution MS.

Spectral data of formula 7 are: ¹H NMR (CDCl₃) δ 6.62 (1H, d, J=16.0, H-9), 6.22 (1H, d, J=16.0, H-8), 5.75 (1 H, d, J=7.5, N-H), 4.75 (1H, dd, J=10.8, 2.2, H-11), 4.28 (1H, d, J=7.5, H-1′), 3.95 (1H, m, H-3′), 3.64 (1H, d, J=10.5, H-3), 3.56 (1H, m, H-5′), 3.16 (1H, dd, J=10.0, 7.5, H-2′), 2.84 (1H, dq, J=10.5, 7.0, H-2), 2.55 (1H, m, H-6), 2.02 (3H, s, NAc), 1.95 (1H, m, H-12), 1.90 (1H, m, H-4′), 1.66 (1H, m, H-5), 1.50 (1H, m, H-12), 1.41 (3H, d, J=7.0, 2-Me), 1.40 (1H, m, H-5), 1.34 (3H, s, 10-Me), 1.25 (1H, m, H-4), 1.22 (1H, m, H-4′), 1.21 (3H, d, J=6.0, H-6′), 1.17 (3H, d, J=7.0, 6-Me), 1.01 (3H, d, J=6.5, 4-Me), 0.89 (3H, t, J=7.2, 12-Me); ¹³C NMR (CDCl₃) δ 204.3 (C-7), 175.1 (C-1), 171.8 (Me—C═O), 149.1 (C-9), 125.3 (C-8), 104.4 (C-1′), 85.4 (C-3), 76.3 (C-11), 75.4 (C-2′), 74.1 (C-10), 68.6 (C-5′), 51.9 (C-3′), 45.0 (C-6), 44.0 (C-2), 38.5 (C-4′), 33.8 (C-5), 33.3 (C-4), 23.1 (Me—C═O), 21.1 (C-12), 20.6 (C-6′), 19.2 (10-Me), 17.5 (6-Me), 17.2 (4-Me), 16.2 (2-Me), 10.6 (12-Me). High-resolution FABMS: calc for C₂₅H₄₃O₈N (M+H)⁺484.2910, found 484.2903.

Spectral data of formula 8 are: ¹H NMR (CDCl₃) 67 6.76 (1H, dd, J=16.0, 5.5, H-9), 6.44 (1H, dd, J=16.0, 1.5, H-8), 5.50 (1H, d, J=6.5, N-H), 4.80 (1H, dd, J=9.0, 2.0, H-11), 4.28 (1H, d, J=7.5, H-1′), 3.95 (1H, m, H-3′), 3.88 (1H, m, H-12), 3.62 (1H, d, J=11.0, H-3), 3.57 (1H, m, H-5′), 3.18 (1H, dd, J=10.0, 7.5, H-2′), 3.06 (1H, m, H-10), 2.86 (1H, dq, J=11.0, 7.0, H-2), 2.54 (1H, m, H-6), 2.04 (3H, s, NAc), 1.98 (1H, m, H-4′), 1.67 (1H, m, H-5), 1.40 (1H, m, H-5), 1.39 (3H, d, J=7.0, 2-Me), 1.25 (1H, m, H-4), 1.22 (1H, m, H-4′), 1.22 (3H, d, J=6.0, H-6′), 1.21 (3H, d, J=6.0, 6-Me), 1.19 (3H, d, J=7.0, 12-Me), 1.16 (3H, d, J=6.5, 10-Me), 1.01 (3H, d, J=6.5, 4-Me); ″¹³C NMR (CDCl₃) δ 205.1 (C-7), 174.6 (C-1), 171.9 (Me—C═O), 147.2 (C-9), 126.2 (C-8), 104.4 (C-1′), 85.3 (C-3), 75.7 (C-11), 75.4 (C-2′), 68.7 (C-5′), 66.4 (C-12), 52.0 (C-3′), 45.1 (C-6), 43.8 (C-2), 38.6 (C-4′) 35.4 (C-10), 34.1 (C-5), 33.4 (C-4), 23.1 (Me—C═O), 21.0 (12-Me), 20.7 (C-6′), 17.7 (6-Me), 17.4 (4-Me), 16.1 (2-Me), 9.8 (10-Me). High-resolution FABMS: calc for C₂₅H₄₃O₈N (M+H)⁺484.2910, found 484.2892.

The fact that compounds of formula (7) and (8) bearing modified desosamine are produced by the desVI-deletion mutant is a thrilling discovery. However, this result is also somewhat surprising since the sugar component in the products is expected to be the aminodeoxy hexose (4). As illustrated in FIG. 27, it is possible that a compound of formula (7) and (8) are derived from the predicted compound of formula (9) and (10), respectively, by a post-synthetic nonspecific acetylation of the attached aminodeoxy sugar. It is also conceivable that N-acetylation of (4) occurs first, followed by coupling of the resulting sugar (11) to the 10-deoxymethynolide (6). Nevertheless, the lack of N-methylation of the sugar component in these new products provides convincing evidence sustaining the assignment of desVI as the N-methyltransferase gene. Most significantly, the production of a compound of formula (7) and (8) by the desVI-deletion mutant attests to the fact that the glycosyltransferase (DesVII) in methymycin/neomethymycin pathway is capable of recognizing and processing sugar substrates other than TDP-desosamine (5).

Since both compounds of formula (7) and (8) are new compounds synthesized in vivo by the S. venezuelae mutant strain, the observed N-acetylation might be a necessary step for self-protection (Cundliffe, 1989). In view of these results, the potential toxicity associated with new macrolide antibiotics produced by genetically engineered microorganisms can be minimized and newly formed antibiotics that have been deactivated (either deliberately or not) during production can be activated. Such an approach can be part of an overall strategy for the development of novel antibiotics using the combinatorial biosynthetic approach. Indeed, purified compounds of formula (7) and (8) are inactive against Streptococcus pyogenes grown on Mueller-Hinton agar plates (Mangahas, 1996), while the controls (a compound of formula (1) and (2)) show clearly visible inhibition zones.

It should be pointed out that a few glycosyltransferases involved in the biosynthesis of antibiotics have been shown to have relaxed specificity towards modified macrolactones (Jacobsen et al., 1997; Marsden et al., 1998; Weber et al., 1991). However, a similar relaxed specificity toward sugar substrates has only been reported for the daunorubicin glycosyltransferase, which is able to recognize a modified daunosamine and catalyze its coupling to the aglycone, ε-rhodomycinone (Madduri et al., 1998). Thus, the fact that the methymycin/neomethymycin glycosyltransferase can also tolerate structural variants of its sugar substrate indicates that at least some glycosyltransferases in antibiotic biosynthetic pathways may be useful to create biologically active hybrid natural products via genetic engineering.

Summary

The appended sugars in macrolide antibiotics are indispensable to the biological activities of these clinically important drugs. Therefore, the development of new antibiotics via a biological combinatorial approach requires detailed knowledge of the biosynthesis of these unusual sugars, as well as the ability to manipulate the biosynthetic genes to create novel sugars that can be incorporated into the final macrolide structures. A targeted deletion of the desVI gene of Streptomyces venezuelae, which has been predicted to encode an N-methyltransferase based on sequence comparison, was prepared to determine whether new methymycin/neomethymycin analogues bearing modified sugars can be generated by altering the desosamine biosynthetic genes. Growth of the S. venezuelae deletion mutant strain resulted in the accumulation of a methymycin/neomethymycin analogue carrying an N-acetylated aminodeoxy sugar. Isolation and characterization of these derivatives not only provide the first direct evidence confirming the identity of desVI as the N-methyltransferase gene, but also demonstrate the feasibility of preparing novel sugars by the gene deletion approach. Most significantly, the results also revealed that the glycosyltransferase of methymycin/neomethymycin exhibits a relaxed specificity towards its sugar substrates.

### Example 8

### Cloning and Sequencing of the Met/Pik Biosynthetic Gene Cluster Materials and Methods

Bacterial Strains and Media. E. coli DH5α was used as a cloning host. E. coli LE392 was the host for a cosmid library derived from S. venezuelae genomic DNA. LB medium was used in E. coli propagation. Streptomyces venezuelae ATCC 15439 was obtained as a freeze-dried pellet from ATCC. Media for vegetative growth and antibiotic production were used as described (Lambalot et al., 1992). Briefly, SGGP liquid medium was for propagation of S. venezuelae mycelia. Sporulation agar (SPA) was used for production of S. venezuelae spores. Methymycin production was conducted in either SCM or vegetative medium and pikromycin production was performed in Suzuki glucose-peptone medium.

Vectors, DNA Manipulation and Cosmid Library Construction. pUC119 was the routine cloning vector, and pNJ1 was the cosmid vector used for genomic DNA library construction. Plasmid vectors for gene disruption were either pGM160 (Muth et al., 1989) or pKC1139 (Bierman et al., 1992). Plasmid, cosmid, and genomic DNA preparation, restriction digestion, fragment isolation, and cloning were performed using standard procedures (Sambrook et al., 1989; Hopwood et al., 1985). The cosmid library was made according to instructions from the Packagene λ-packaging system (Promega).

DNA Sequencing and Analysis. An Exonuclease III (ExoIII) nested deletion series combined with PCR-based double stranded DNA sequencing was employed to sequence the pik cluster. The ExolIl procedure followed the Erase-a-Base protocol (Stratagene) and DNA sequencing reactions were performed using the Dye Primer Cycle Sequencing Ready Reaction Kit (Applied Biosystems). The nucleotide sequences were read from an ABI PRISM 377 sequencer on both DNA strands. DNA and deduced protein sequence analyses were performed using GeneWorks and GCG sequence analysis package. All analyses were performed using the specific program default parameters.

Gene Disruption. A replicative plasmid-mediated homologous recombination approach was developed to conduct gene disruption in S. venezuelae. Plasmids for insertional inactivation were constructed by cloning a kanamycin resistance marker into target genes, and plasmid for gene deletion/replacement was constructed by replacing the target gene with a kanamycin or thiostrepton resistance gene in the plasmid. Disruption plasmids were introduced into S. venezuelae by either PEG-mediated protoplast transformation (Hopwood et al., 1985) or RK2-mediated conjugation (Bierman et al., 1992). Then, spores from individual transformants or transconjugants were cultured on non-selective plates to induce recombination. The cycle was repeated three times to enhance the opportunity for recombination. Double crossovers yielding targeted gene disruption mutants were selected and screened using the appropriate combination of antibiotics and finally confirmed by Southern hybridization.

Antibiotic Extraction and Analysis. Methymycin, pikromycin, and related compounds were extracted following published procedures (Cane et al., 1993). Thin layer chromatography (TLC) was routinely used to detect methymycin, neomethymycin, narbomycin and pikromycin. Further purification was conducted using flash column chromatography and HPLC, and the purified compounds were analyzed by ¹H, ¹³C NMR spectroscopy and MS spectrometry.

Results

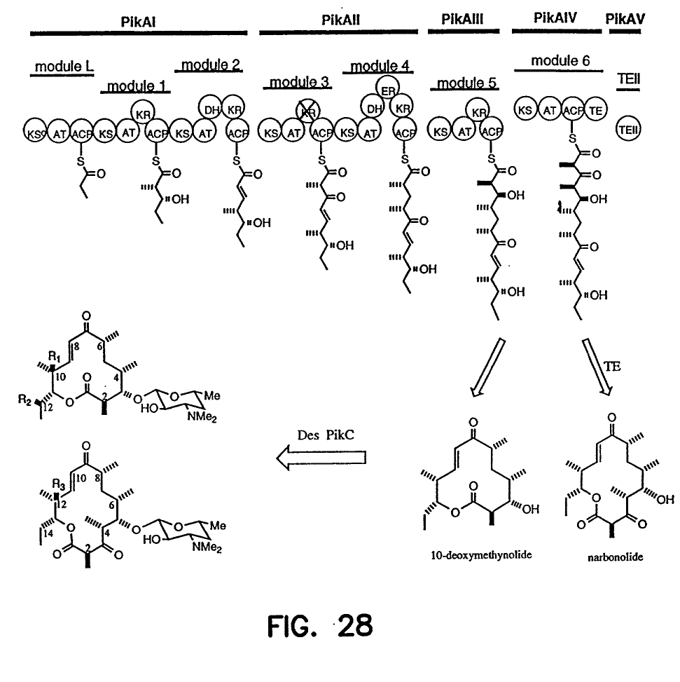

Cloning and Identification of the pik Cluster. Heterologous hybridization was used to identify genes for methymycin, neomethymycin, narbomycin and pikromycin biosynthesis in S. venezuelae. Initial Southern blot hybridization analysis using a type I PKS DNA probe revealed two multifunctional PKS clusters of uncharacterized function in the genome. Since these four antibiotics are all comprised of an identical desosamine residue, a tylAI α-D-glucose-1-phosphate thymidylyltransferase DNA probe (for mycaminose/mycorose/mycinose biosynthesis in the tylosin pathway) (Merson-Davies et al., 1994) was used to locate the corresponding biosynthetic gene cluster(s). This analysis established that only one of the PKS pathways contained a cluster of desosamine biosynthetic genes. Nine overlapping cosmid clones were isolated spanning over 80 kilobases (kb) on the bacterial chromosome that encompassed the entire gene cluster (pik) for methymycin, neomethymycin, narbomycin and pikromycin biosynthesis (FIG. 28). Through subsequent gene disruption, the other PKS cluster (vep, devoid of linked desosamine biosynthetic genes) was found to play no role in production of methymycin, neomethymycin, narbomycin or pikromycin.

Nucleotide Sequence of the pik Cluster. The nucleotide sequence of the pik cluster was completely determined and shown to contain 18 open reading frames (ORFs) that span approximately 60 kb. Central to the cluster are four large ORFs, pikAI, pikAII, pikAIII, and pikAIV, encoding a multifunctional PKS (FIG. 28). Analysis of the six modules comprising the pik PKS indicated that it would specify production of narbonolide, the 14-membered ring aglycone precursor of narbomycin and pikromycin (FIG. 28).

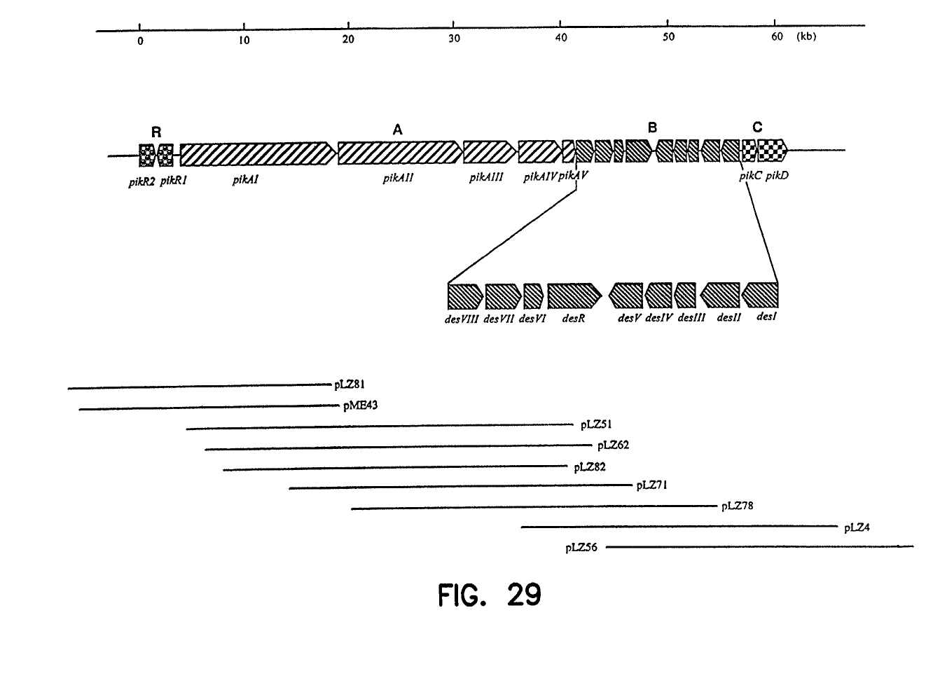

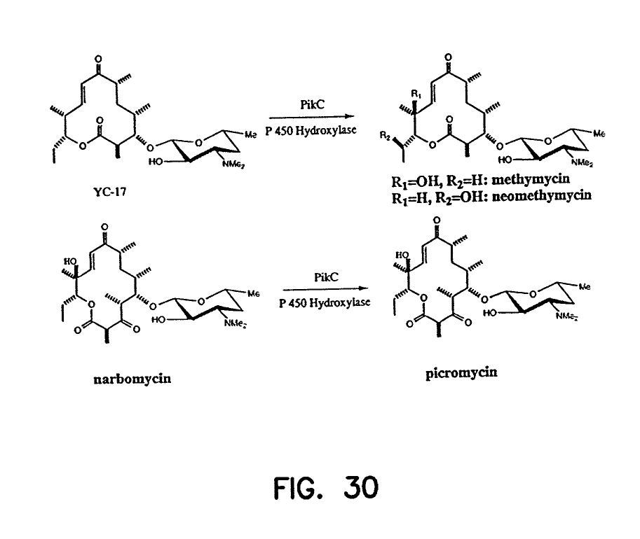

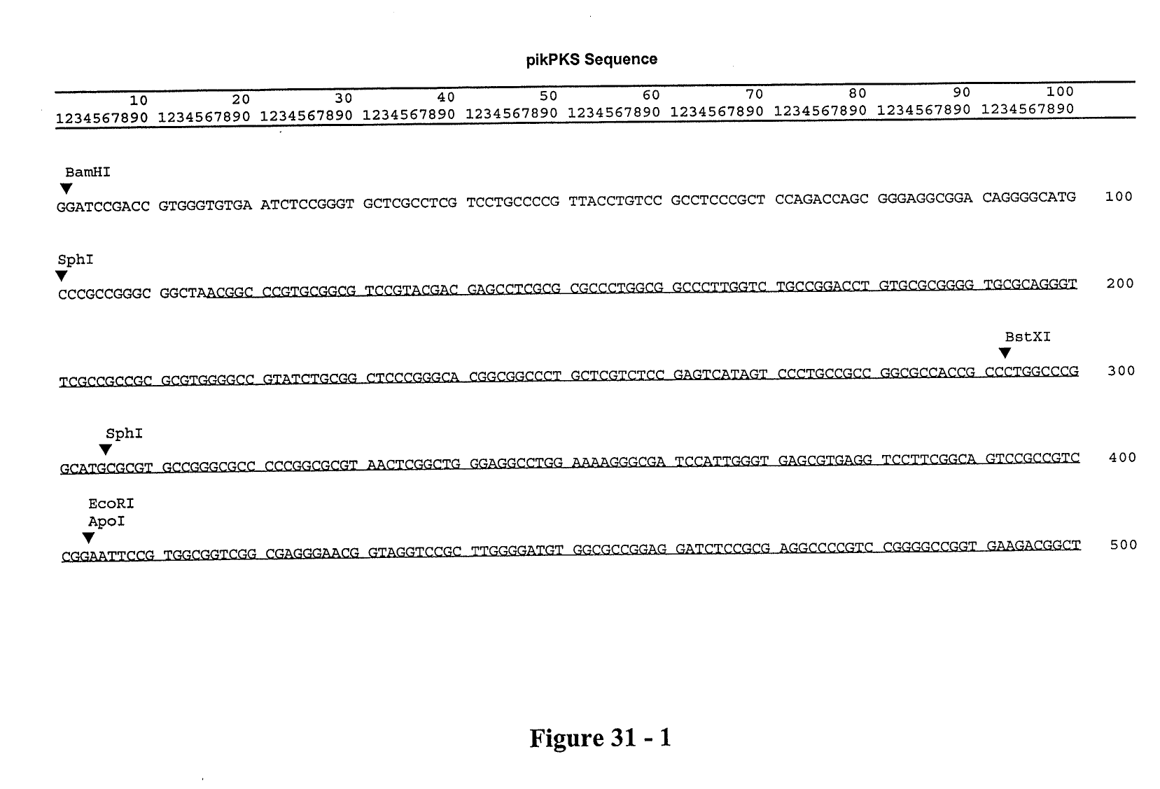

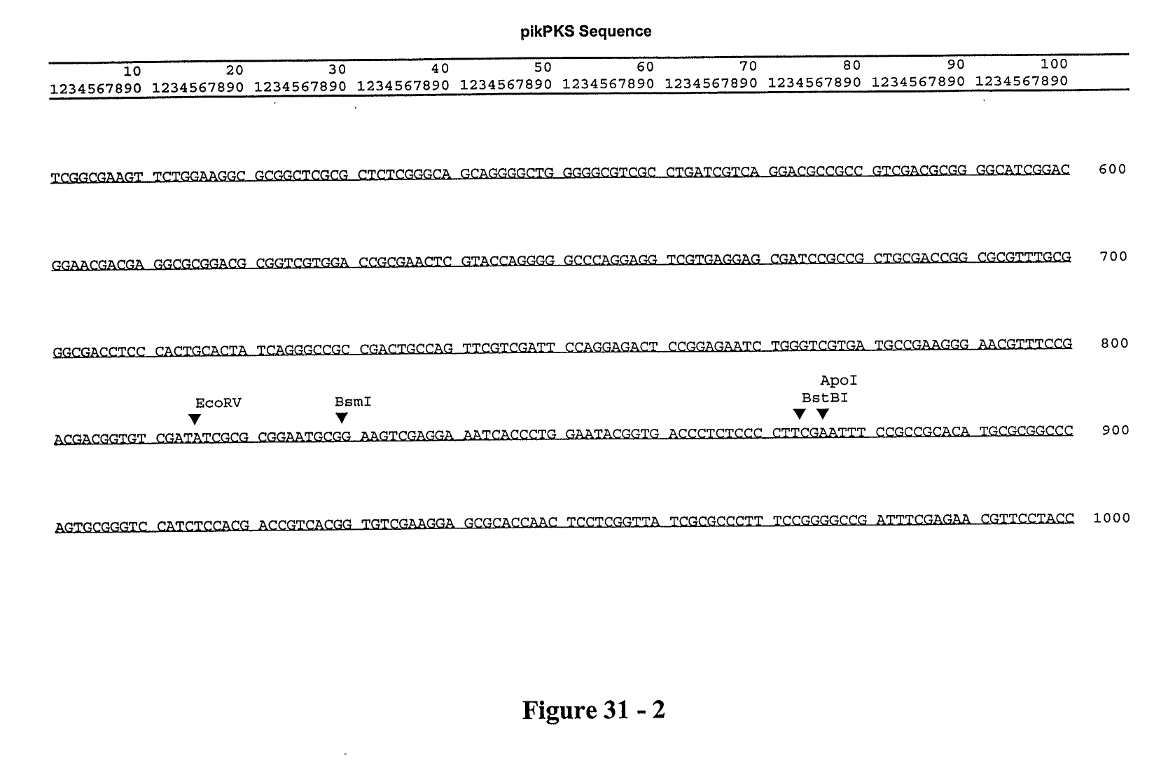

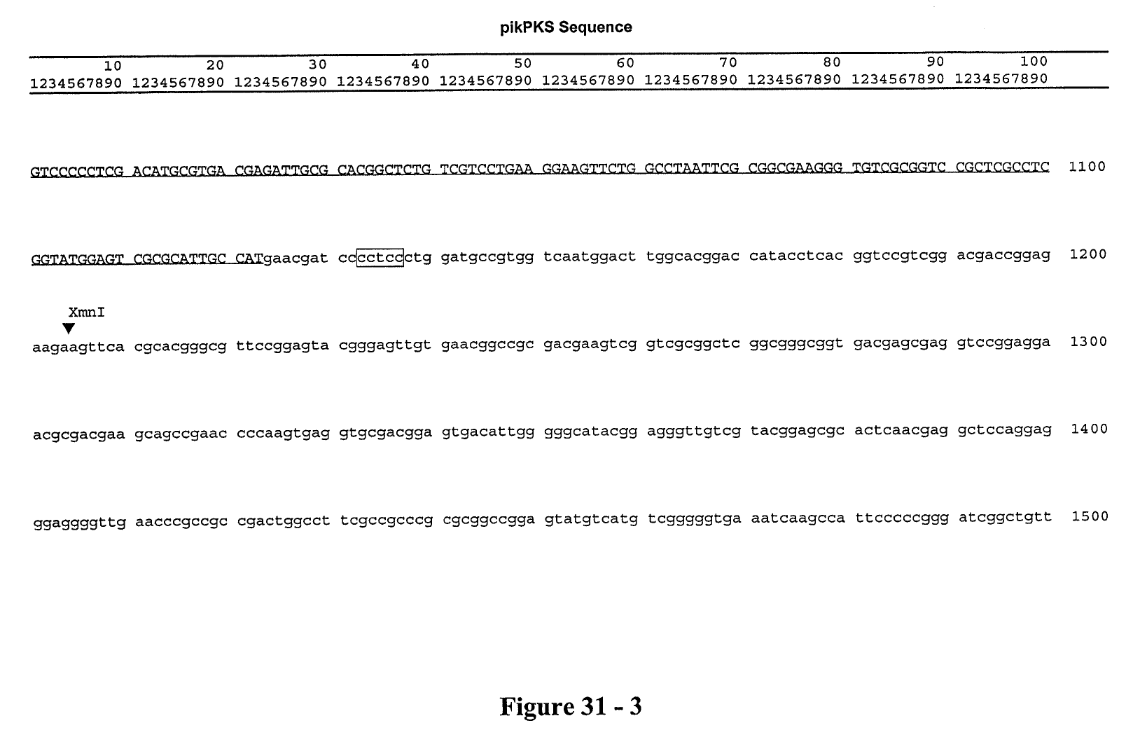

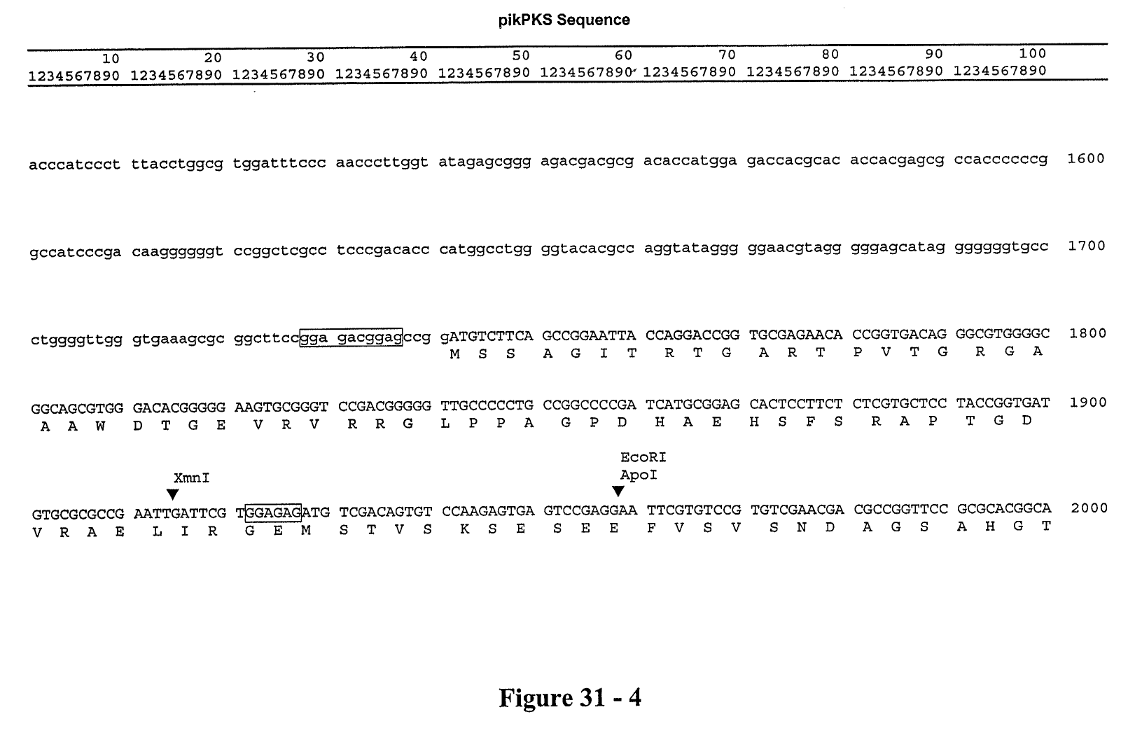

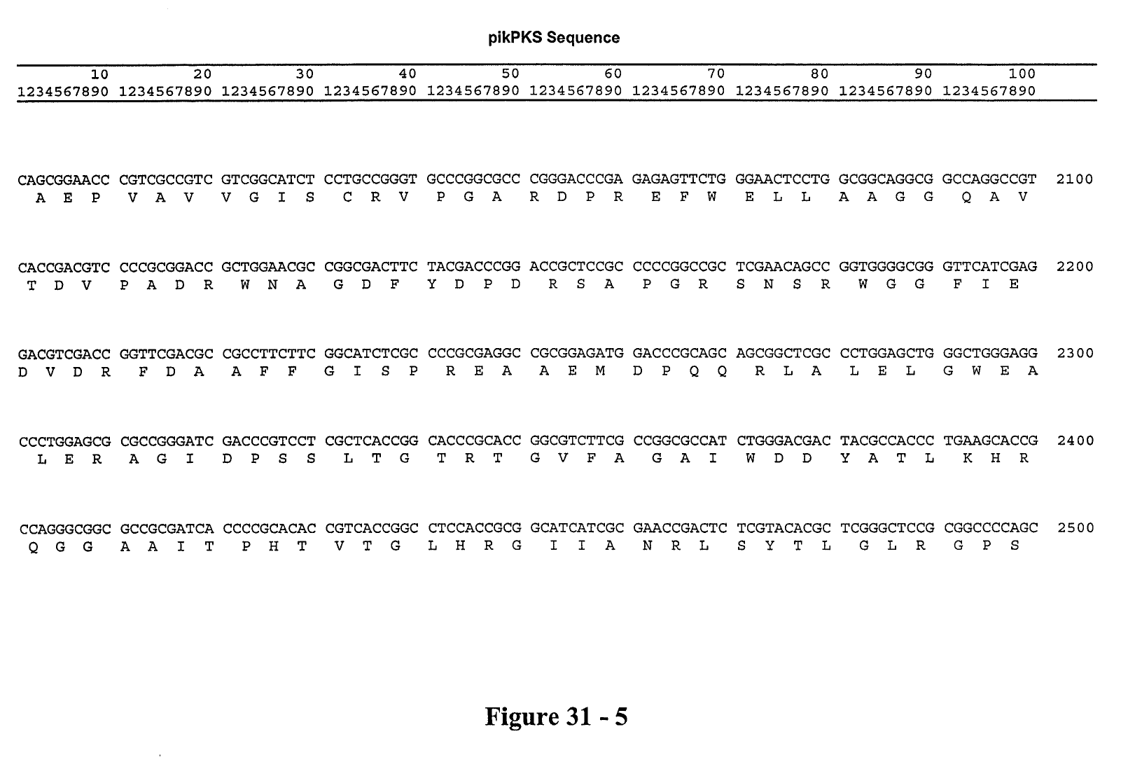

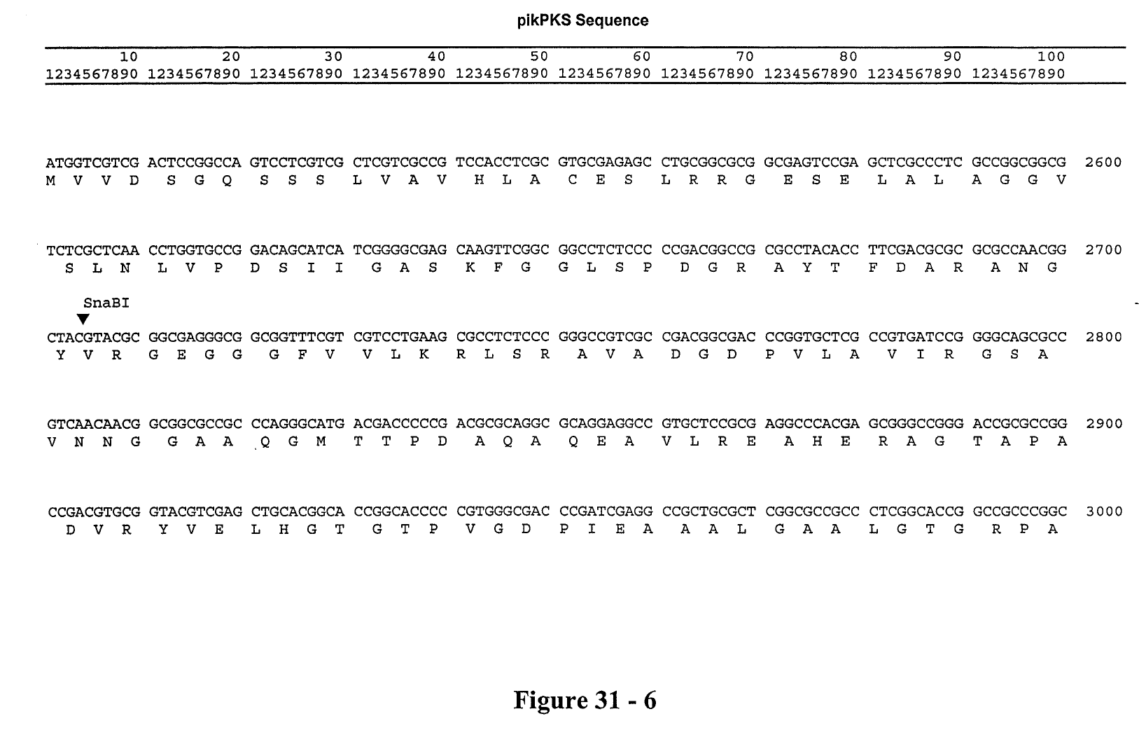

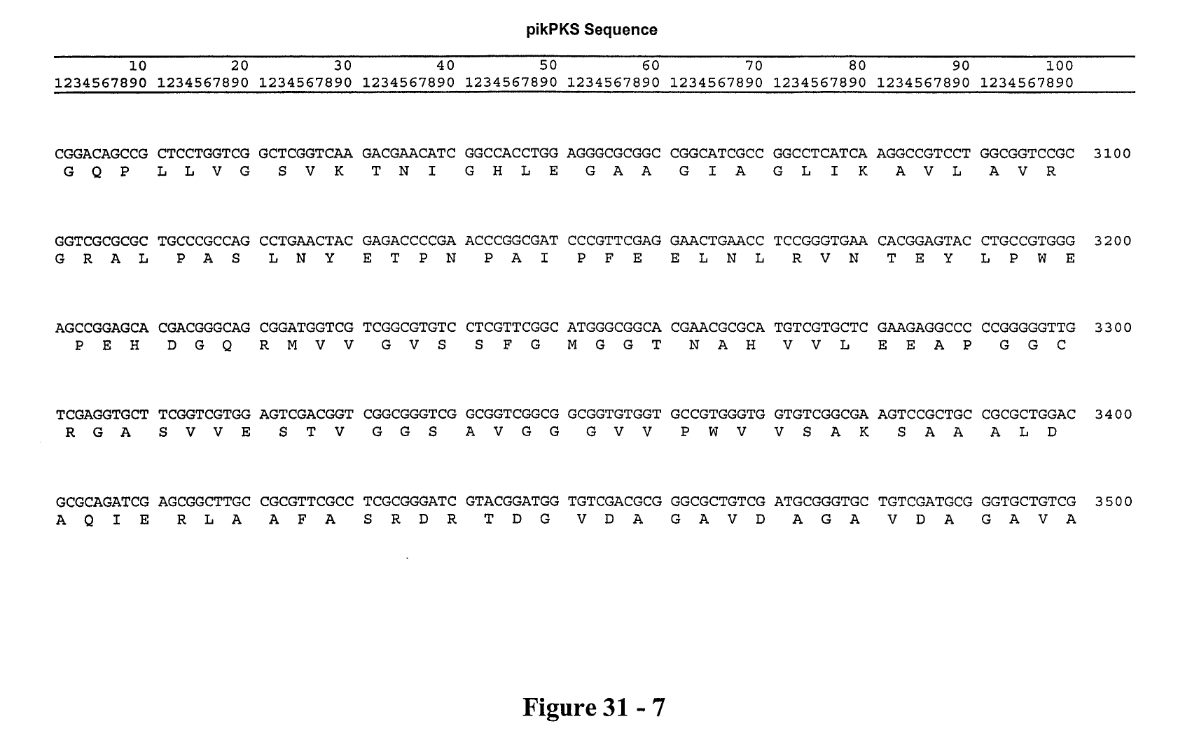

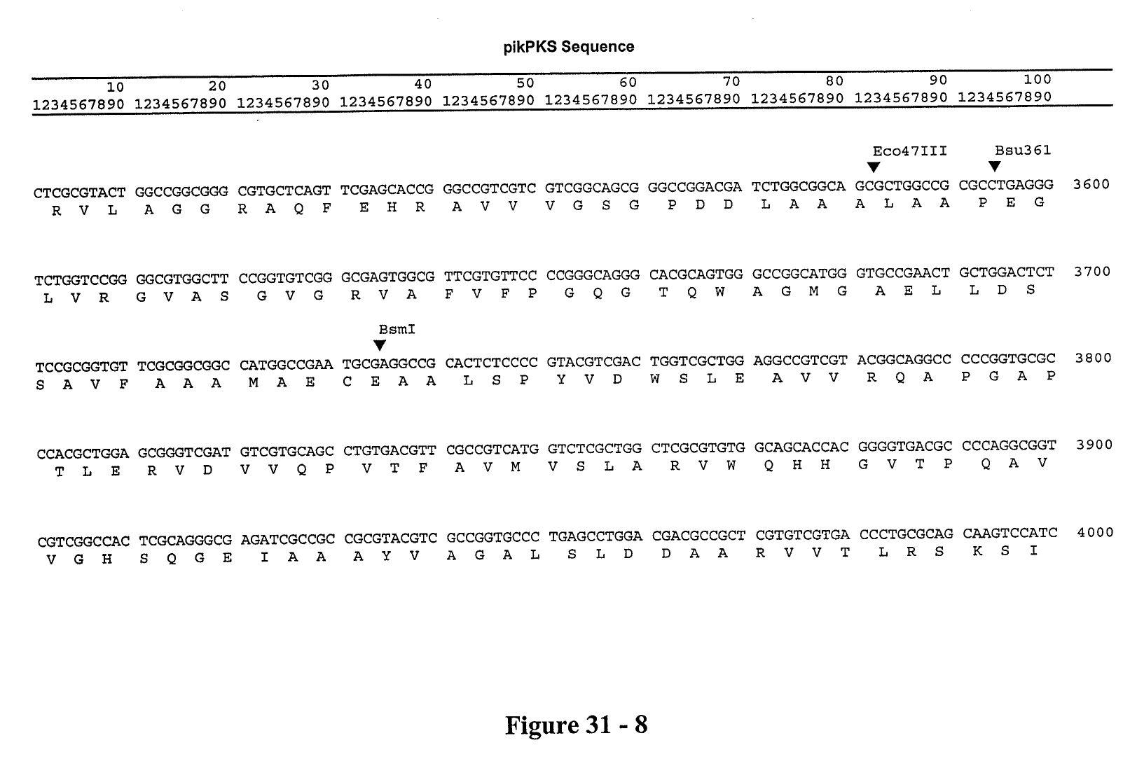

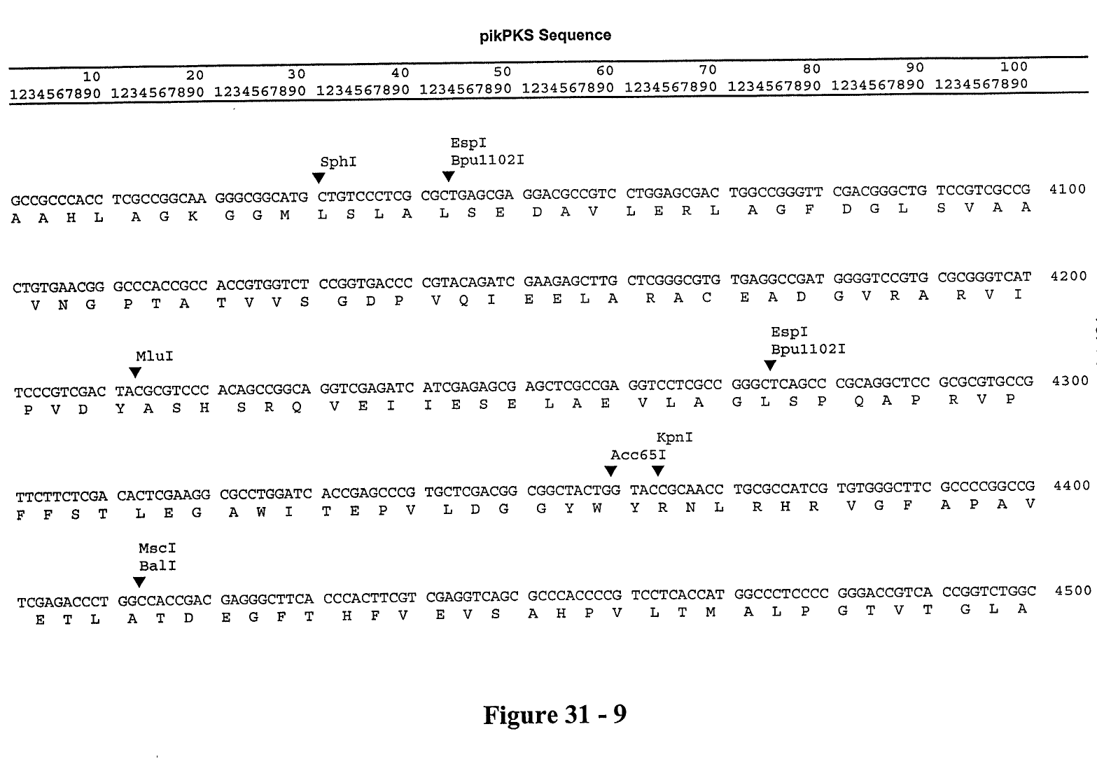

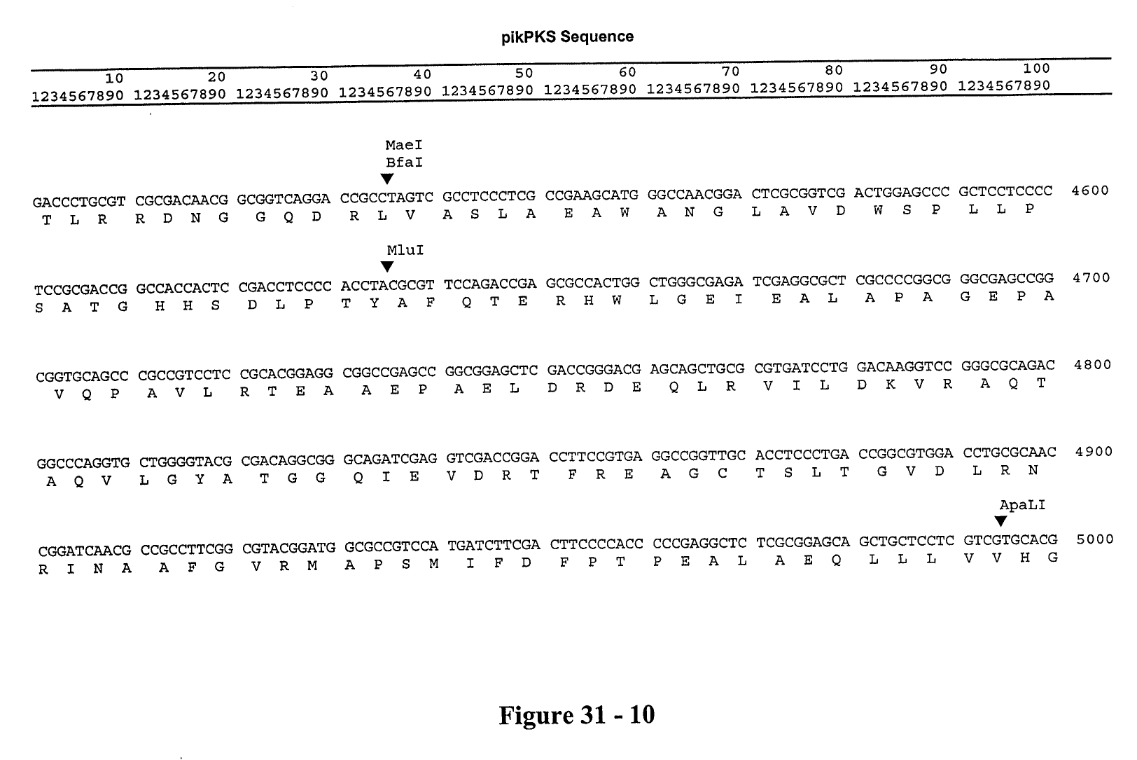

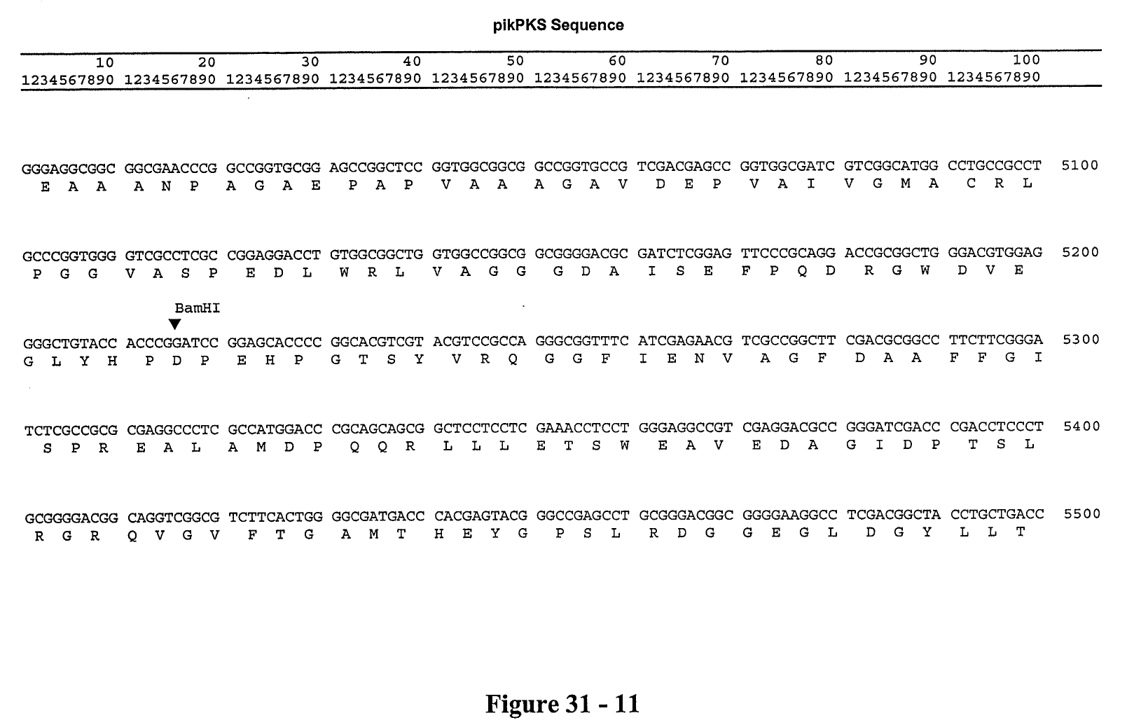

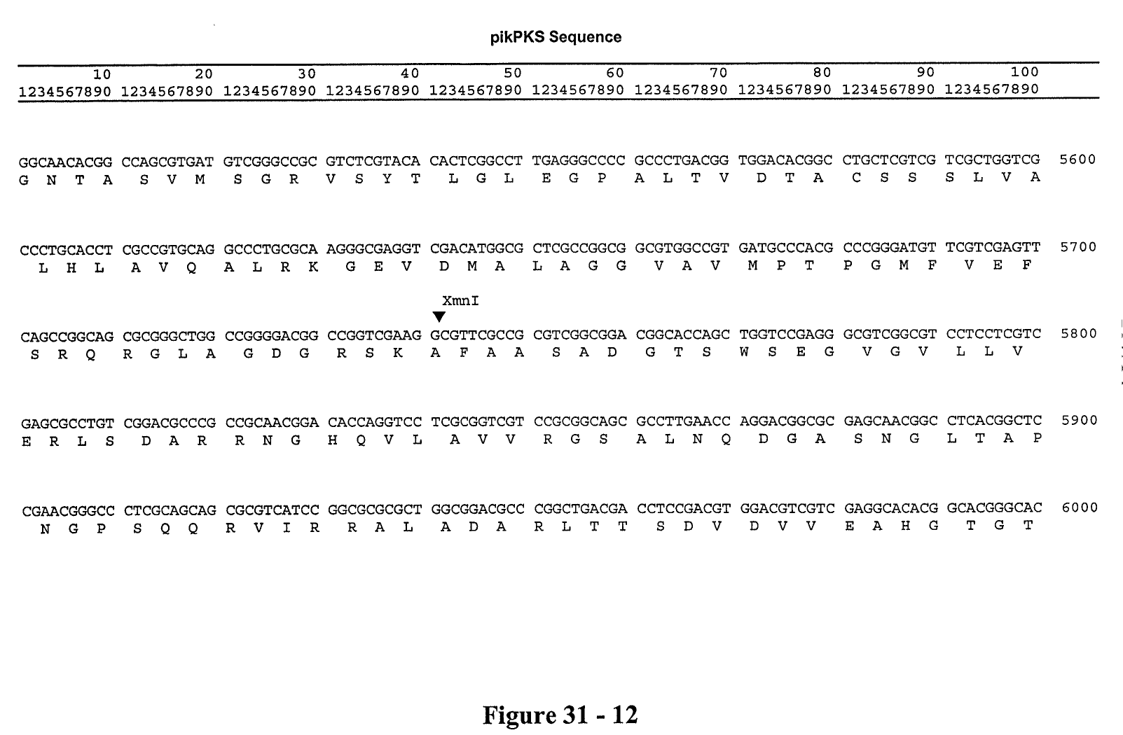

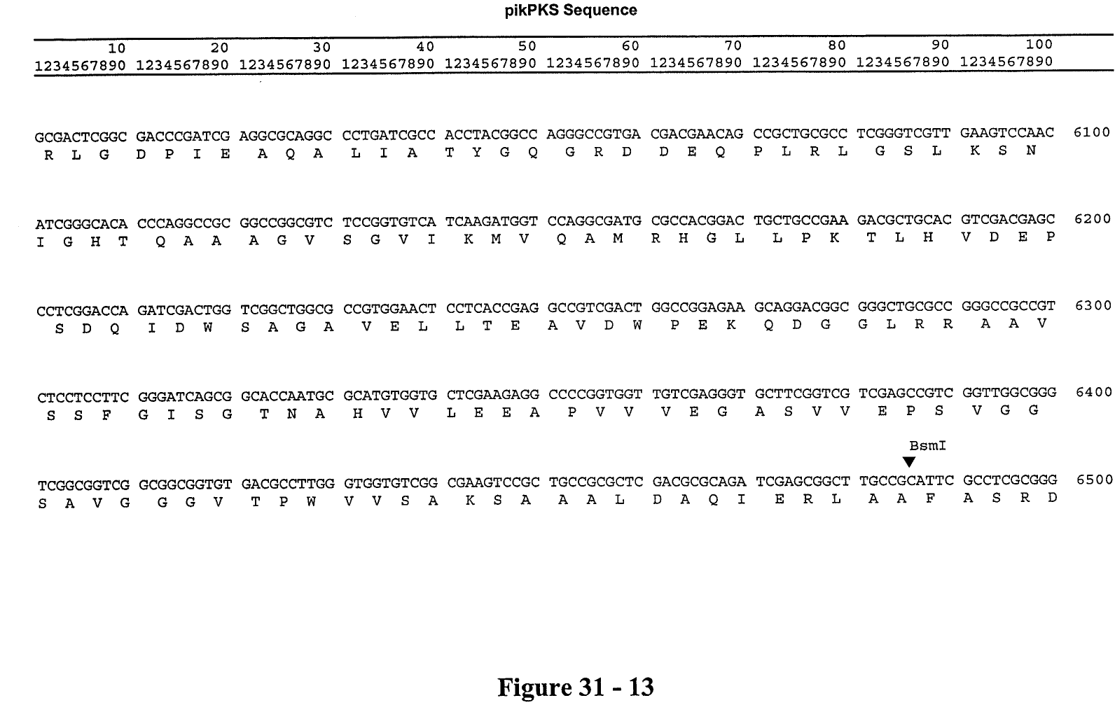

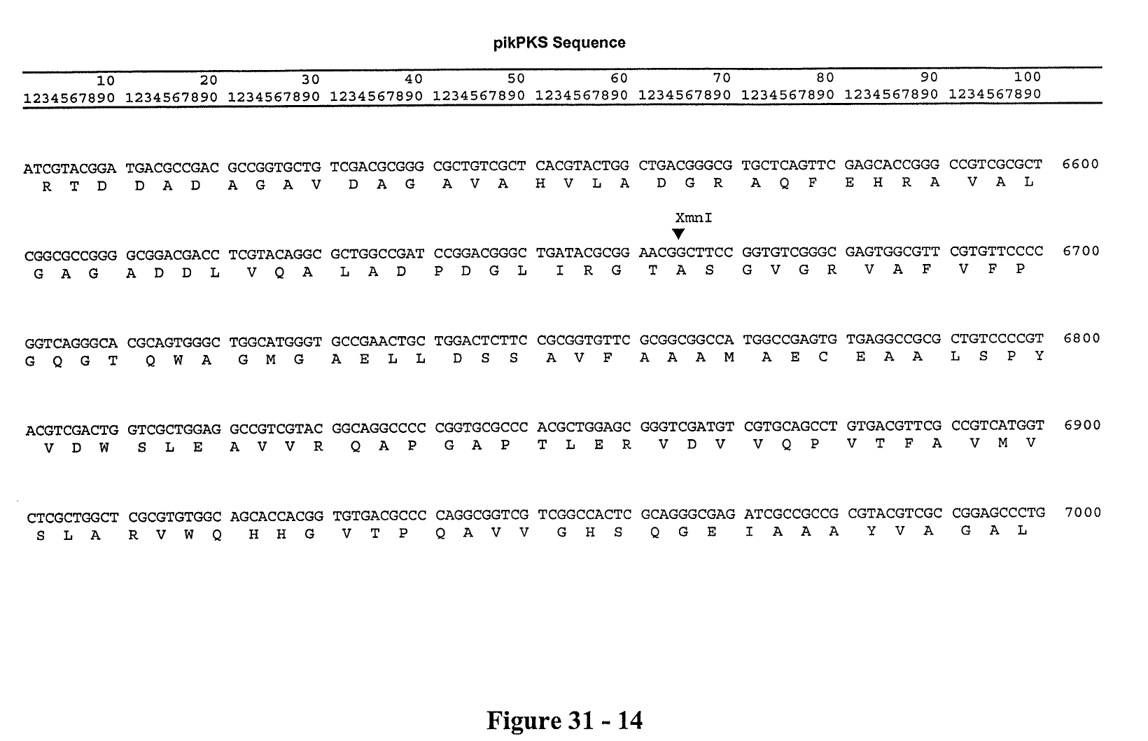

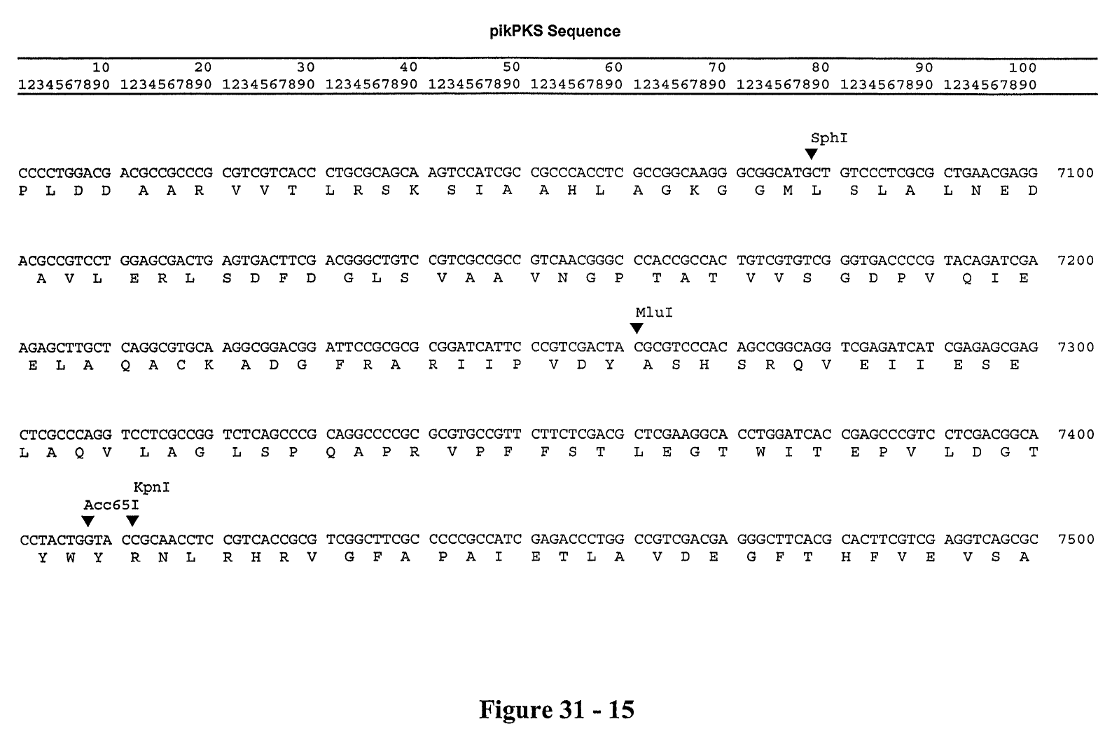

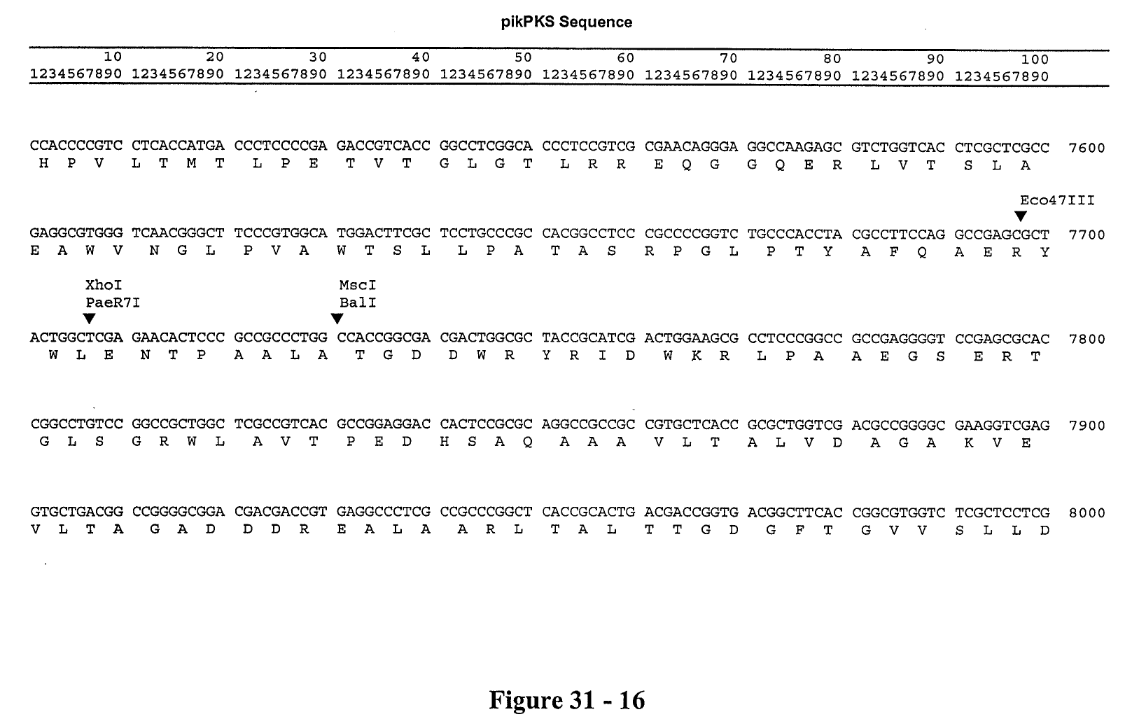

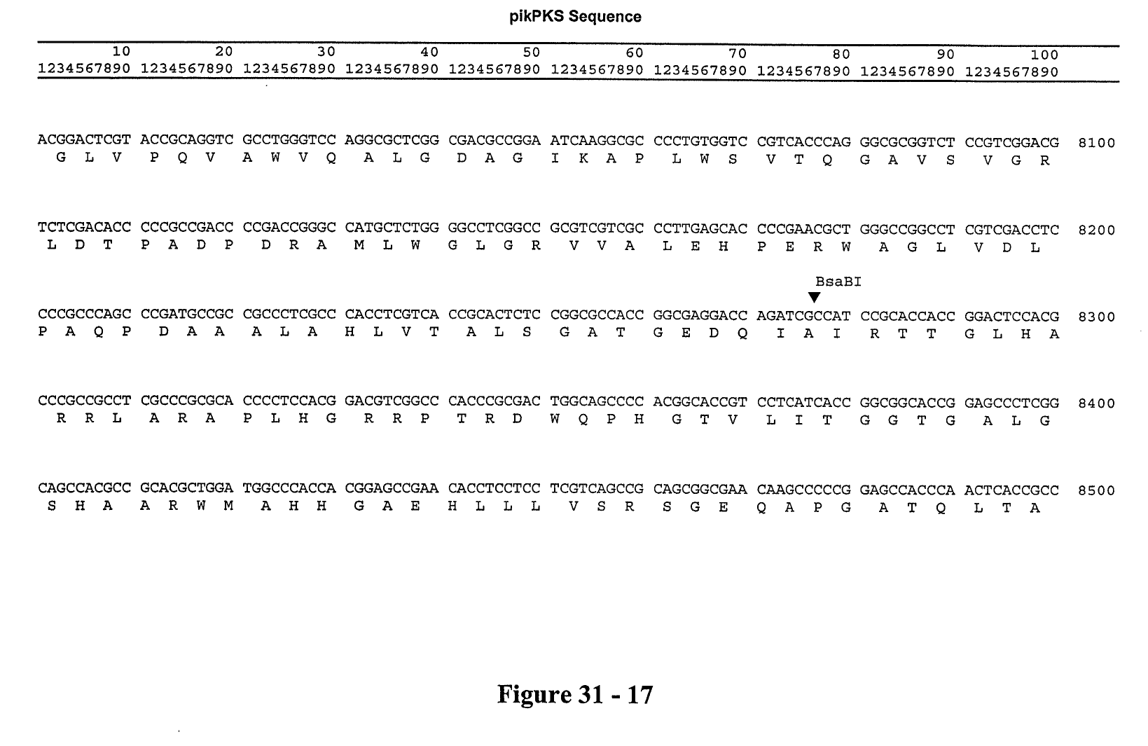

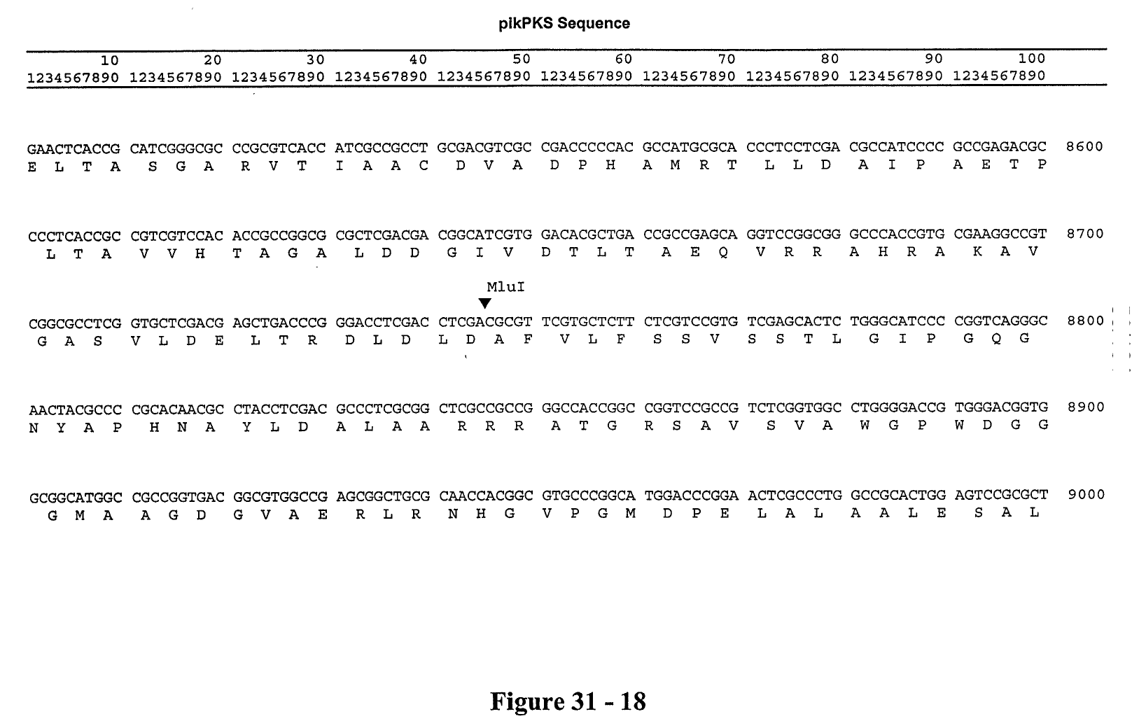

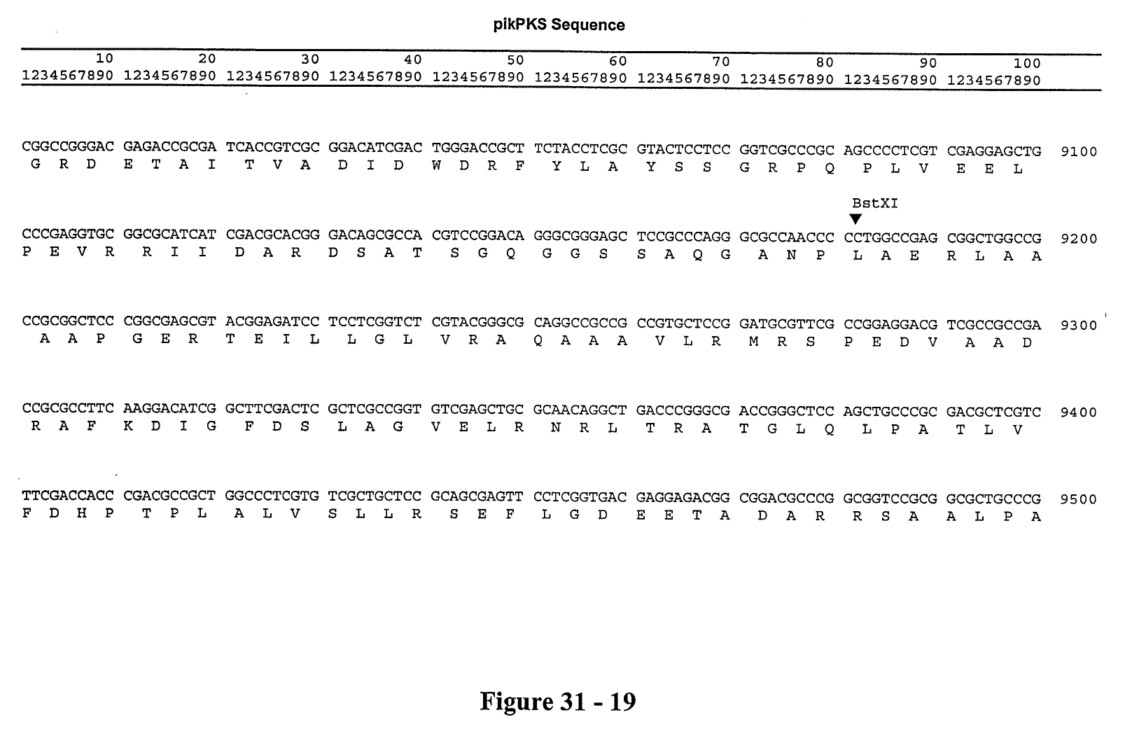

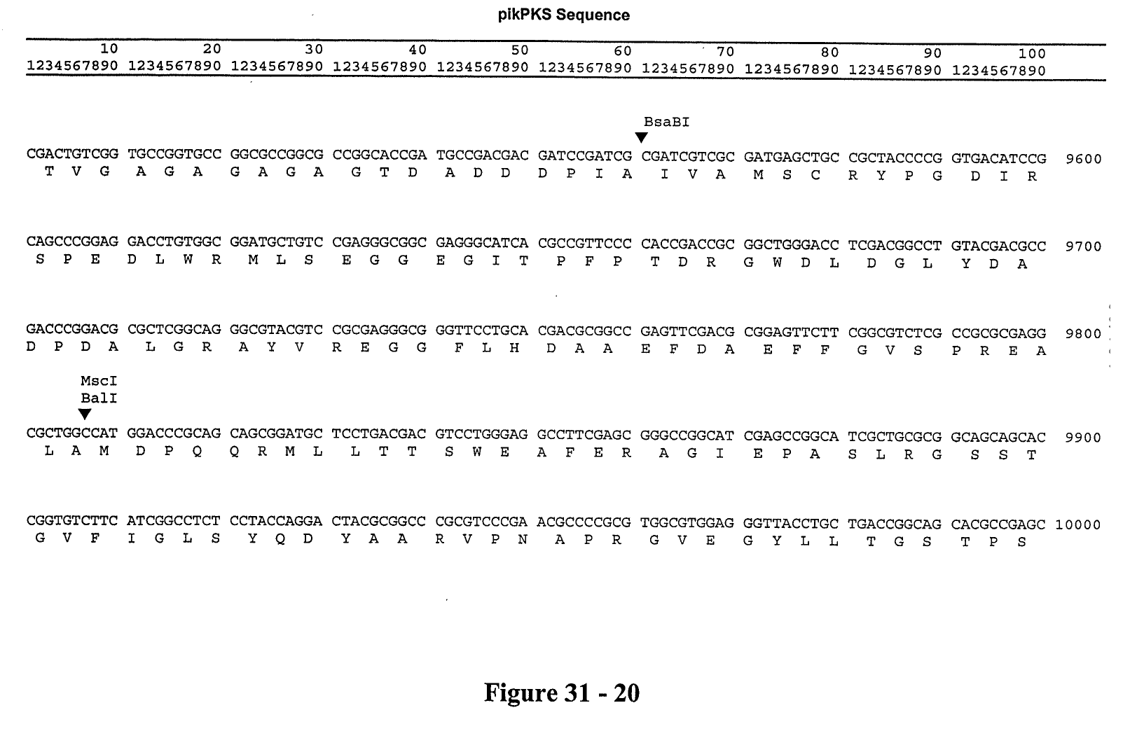

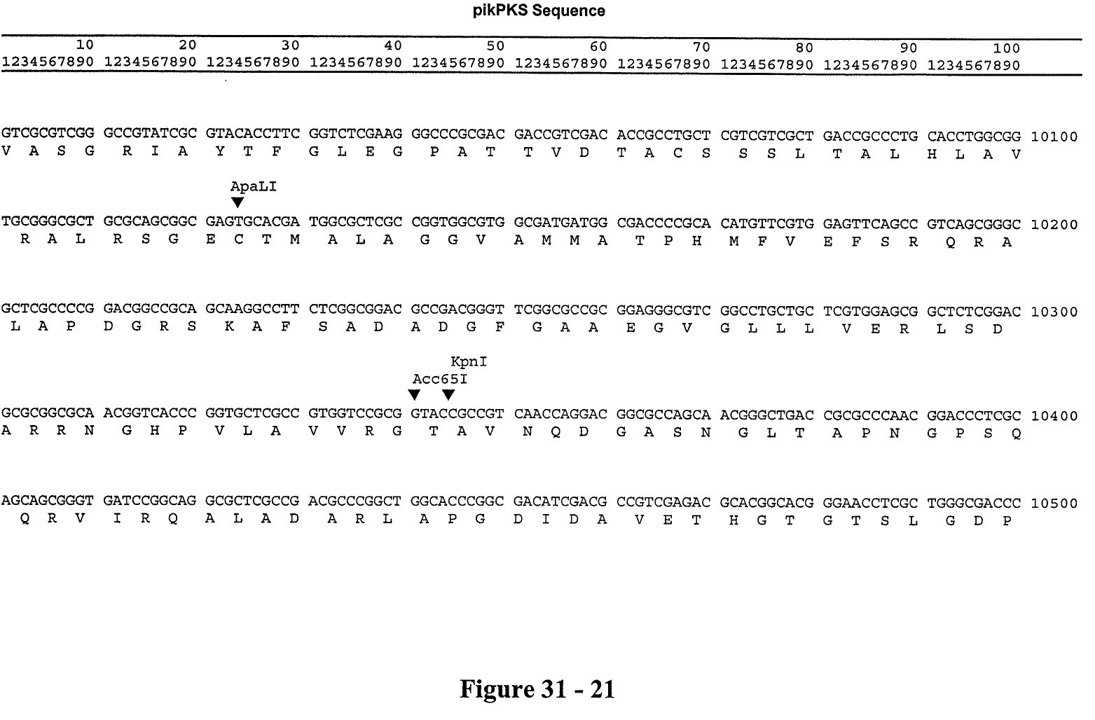

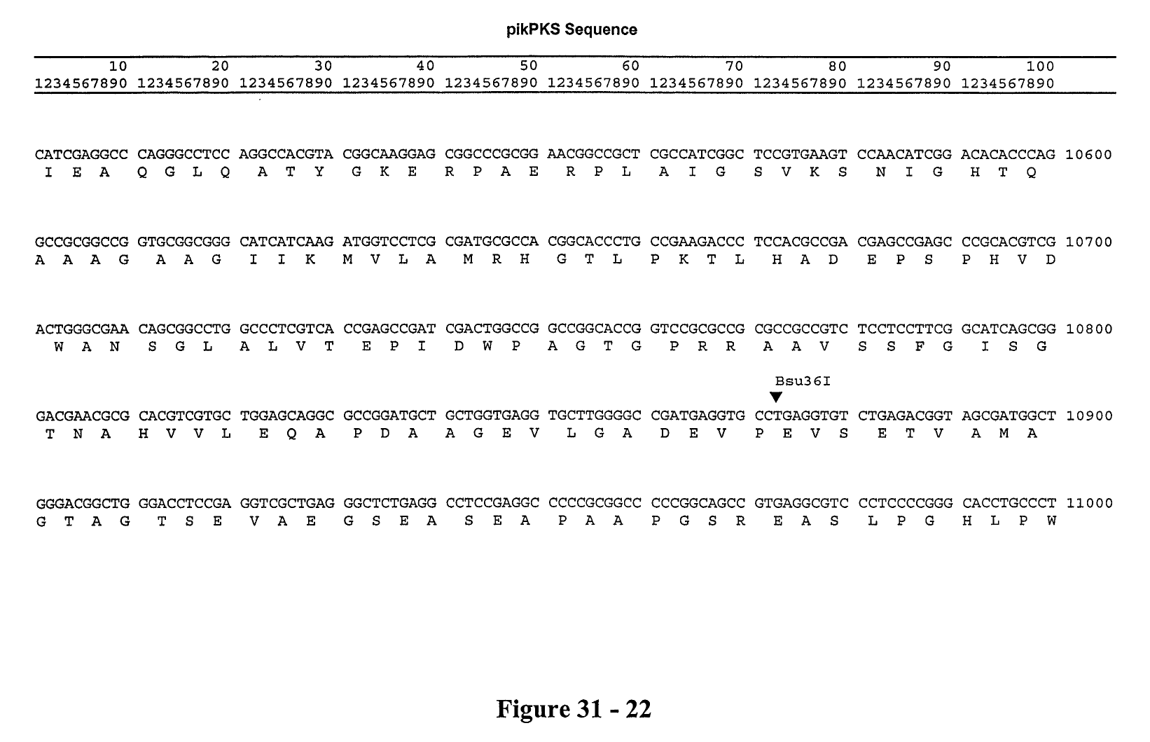

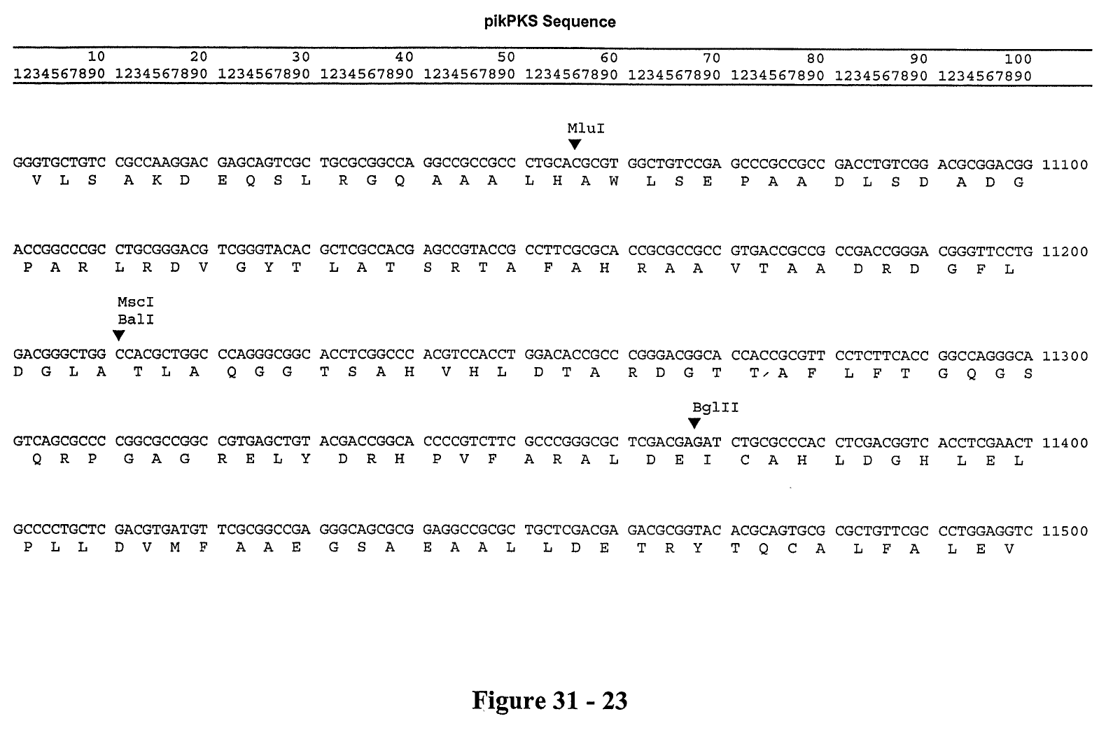

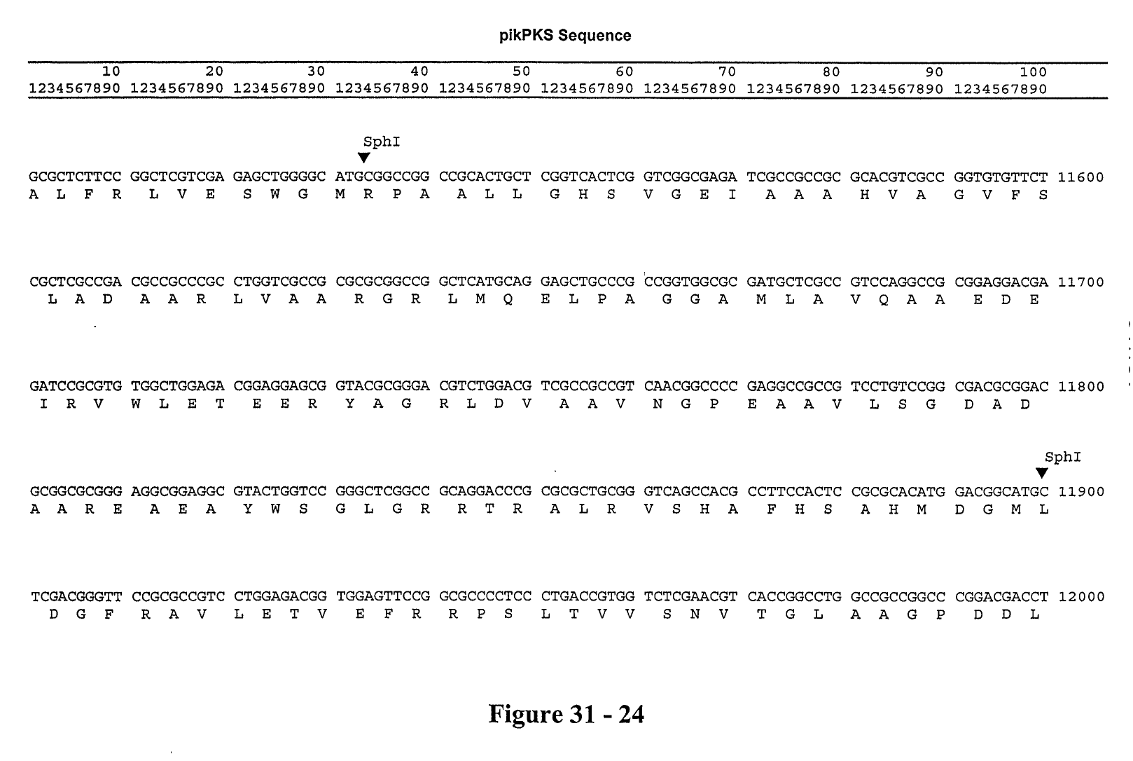

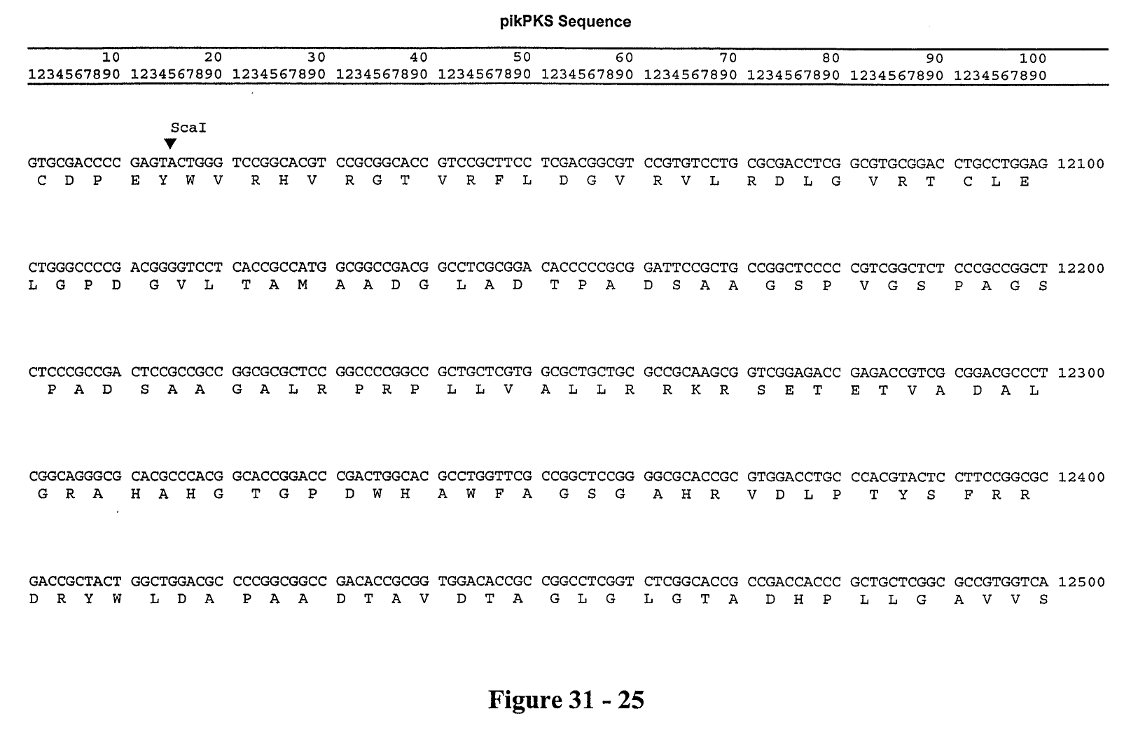

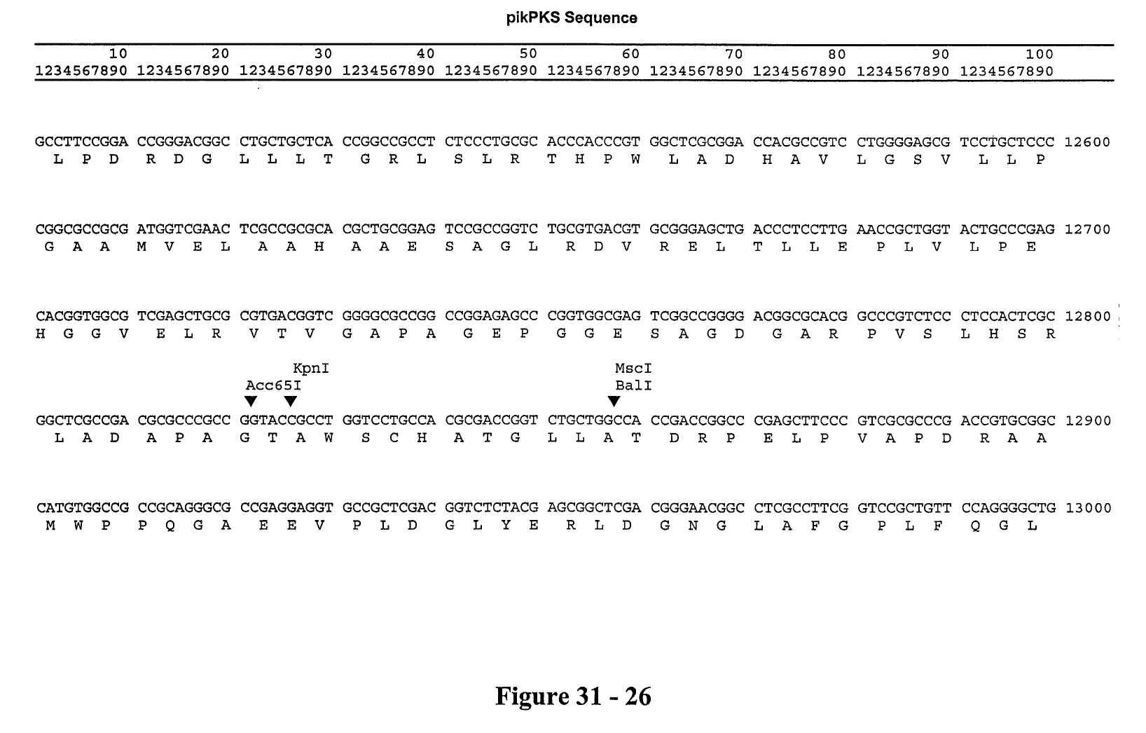

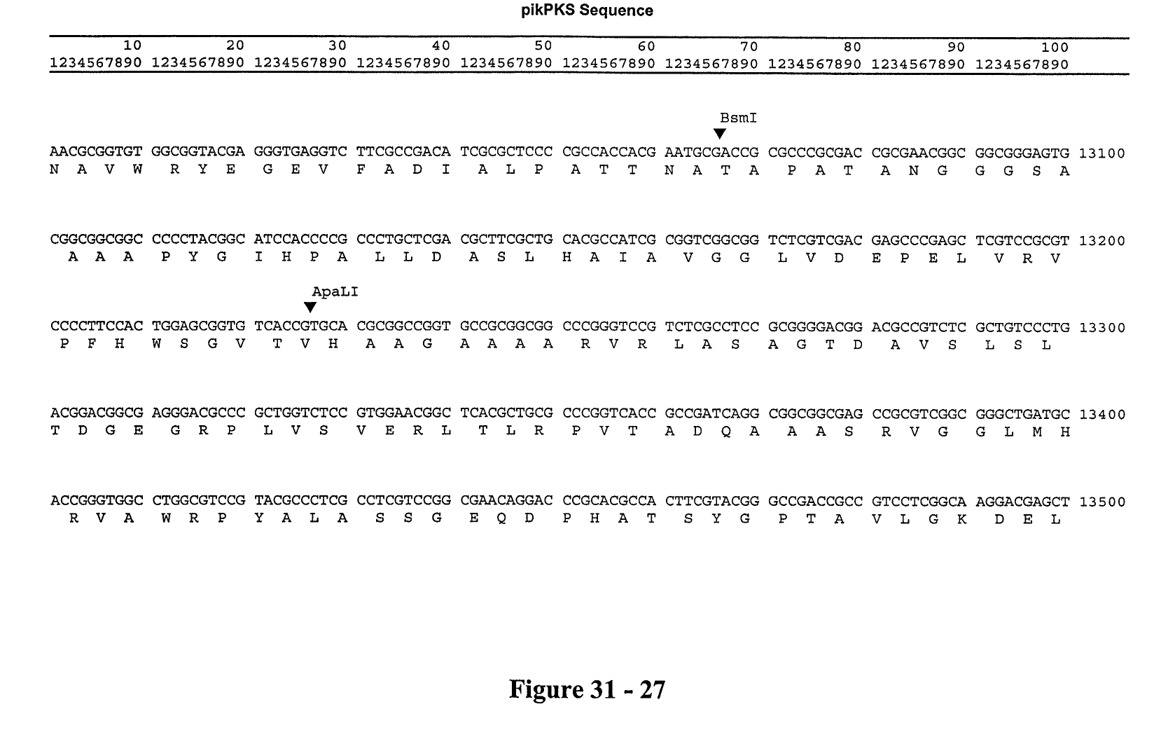

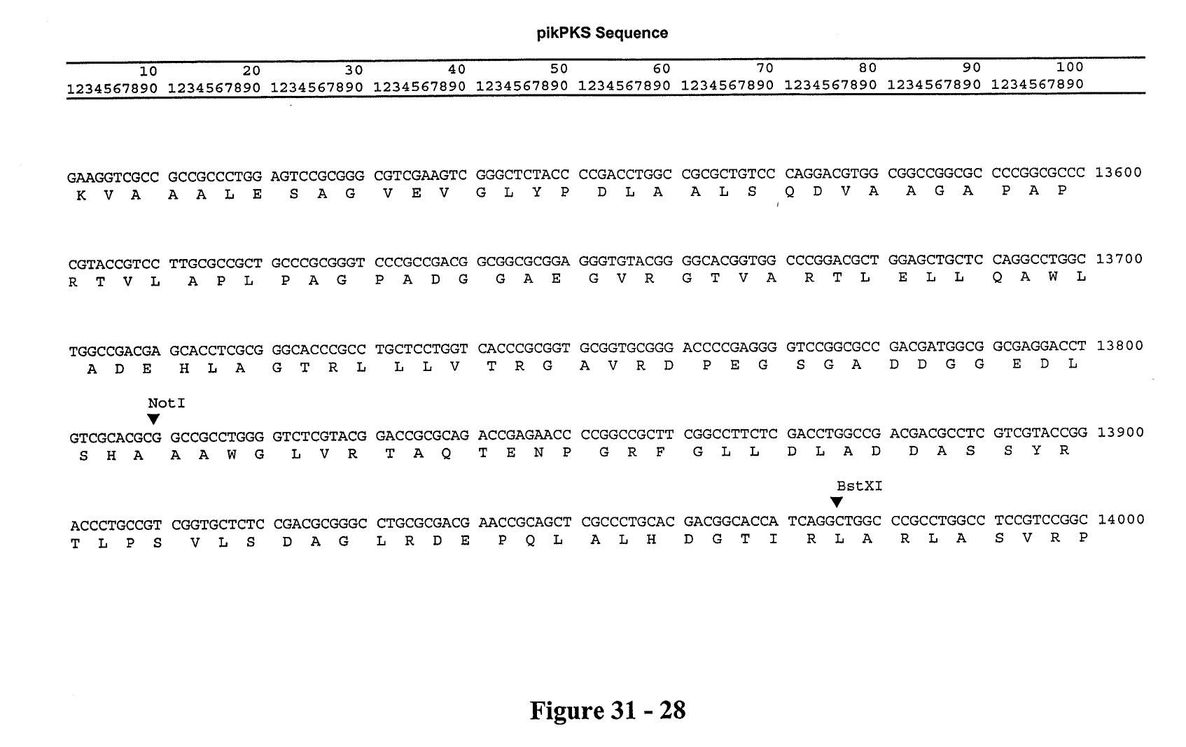

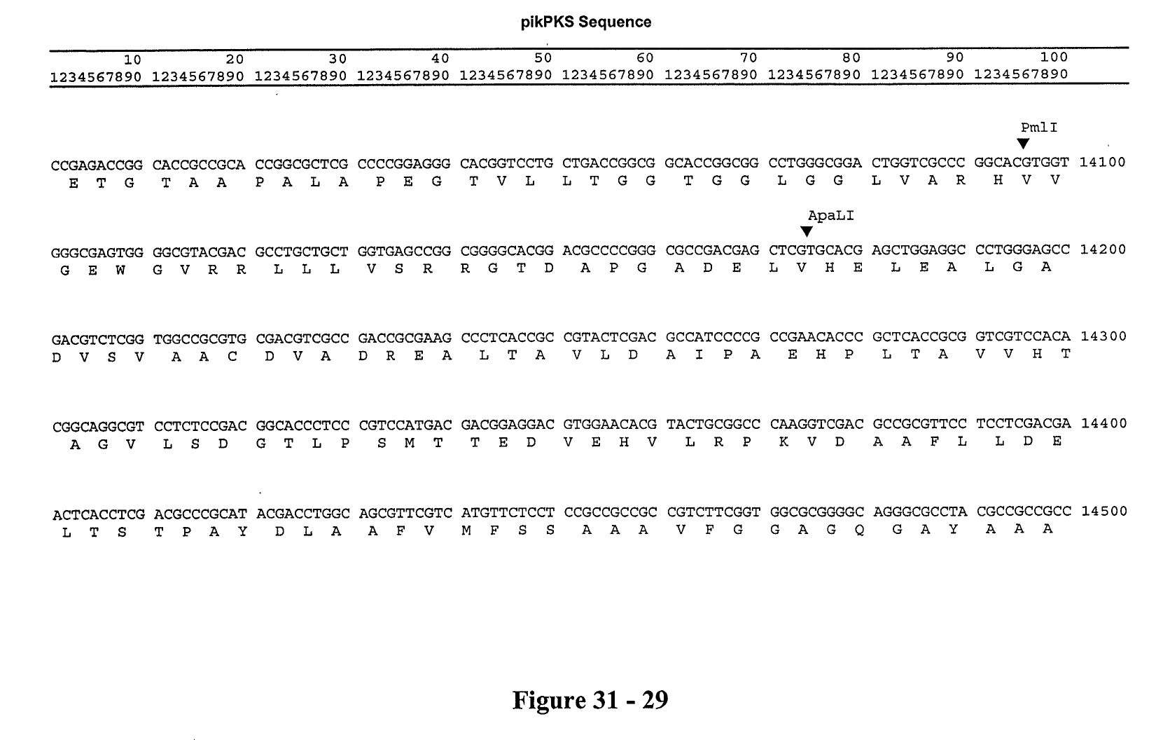

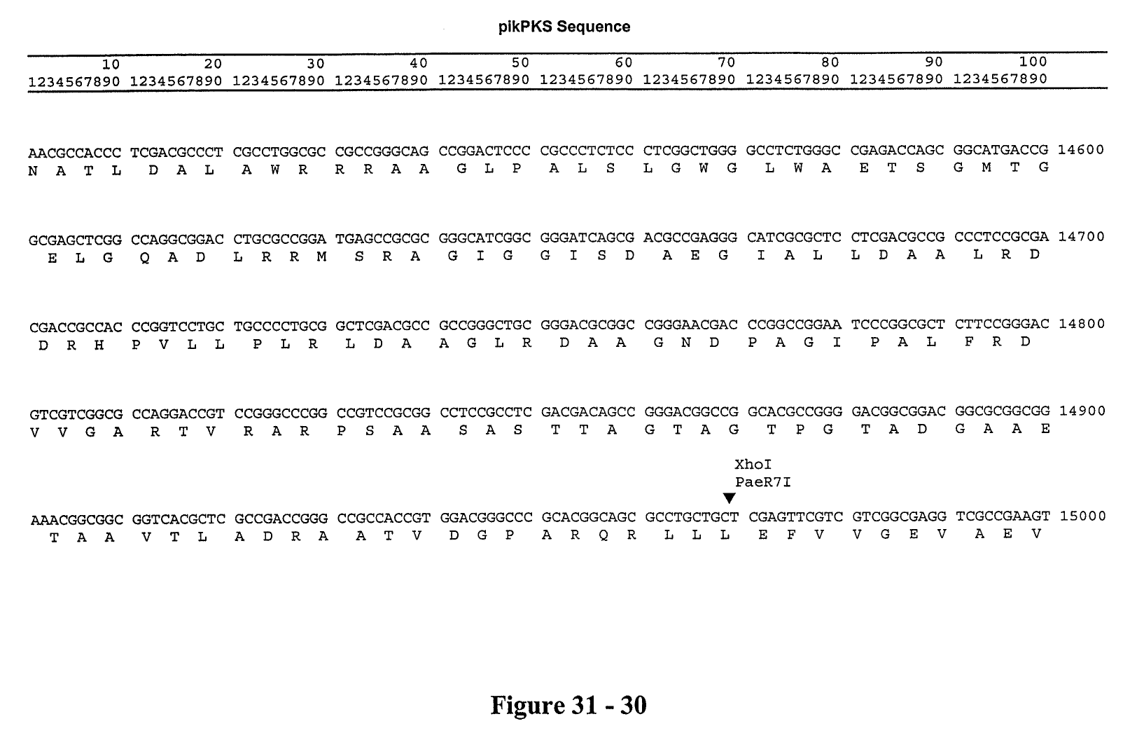

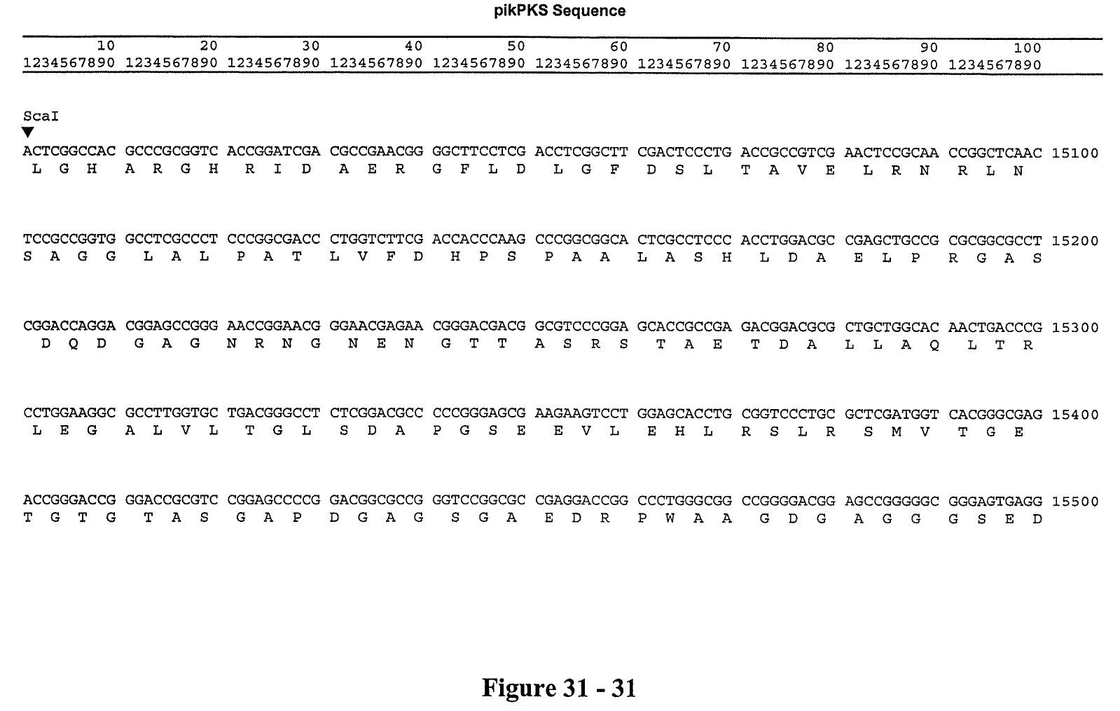

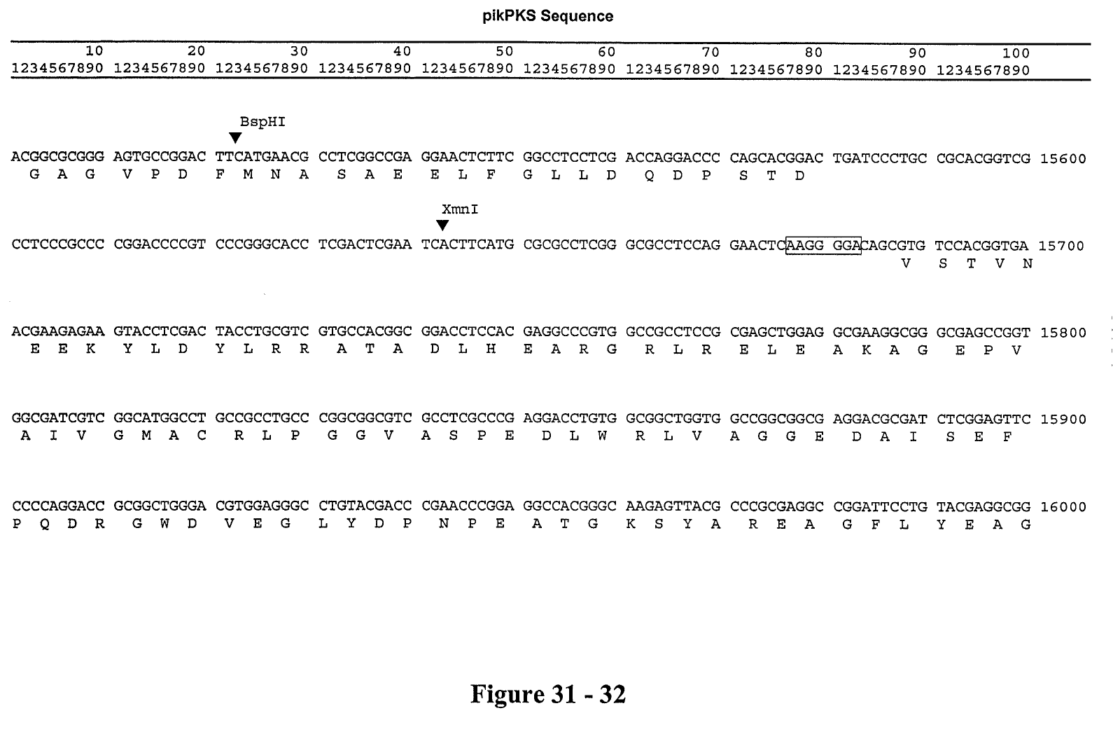

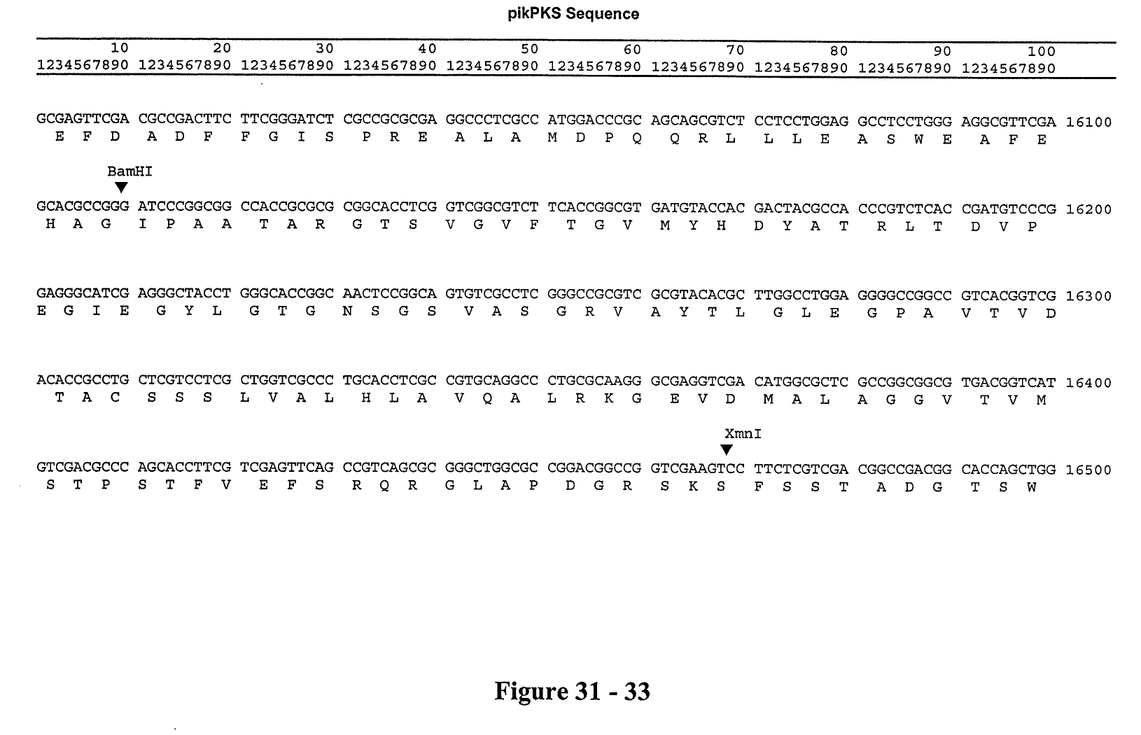

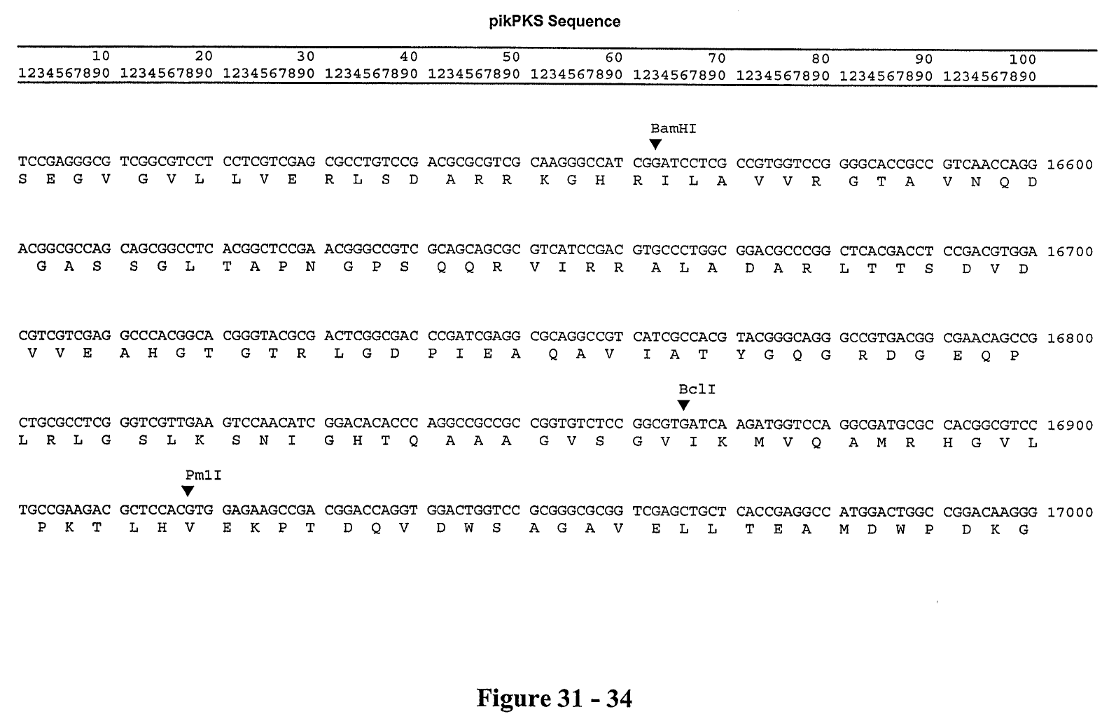

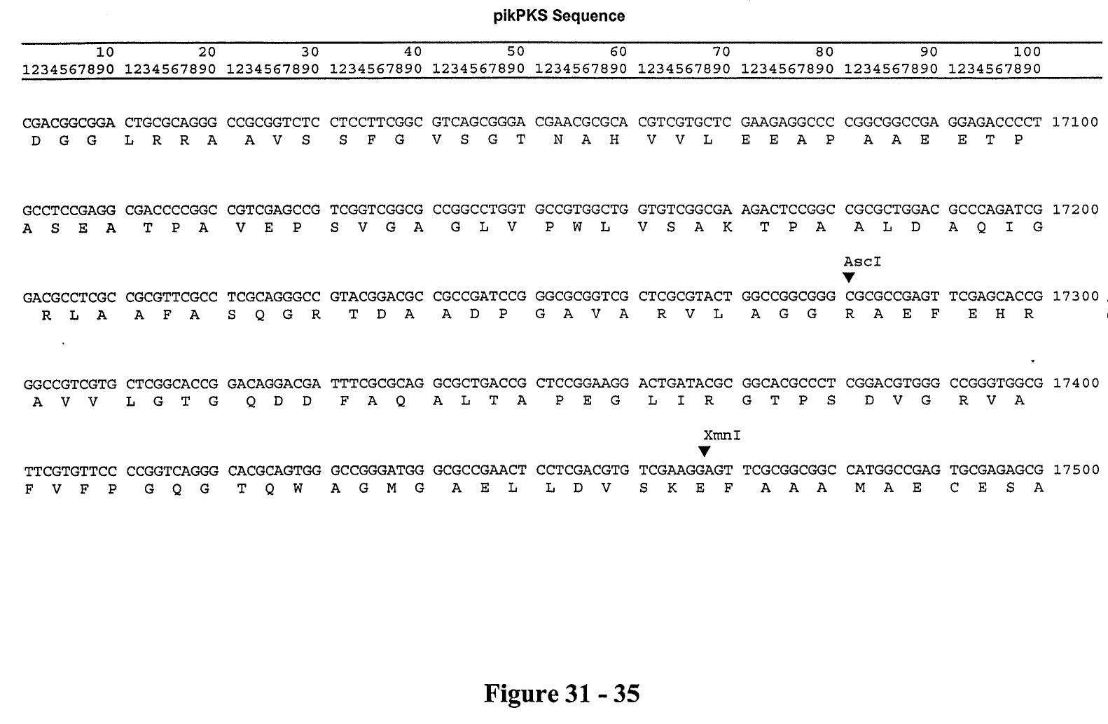

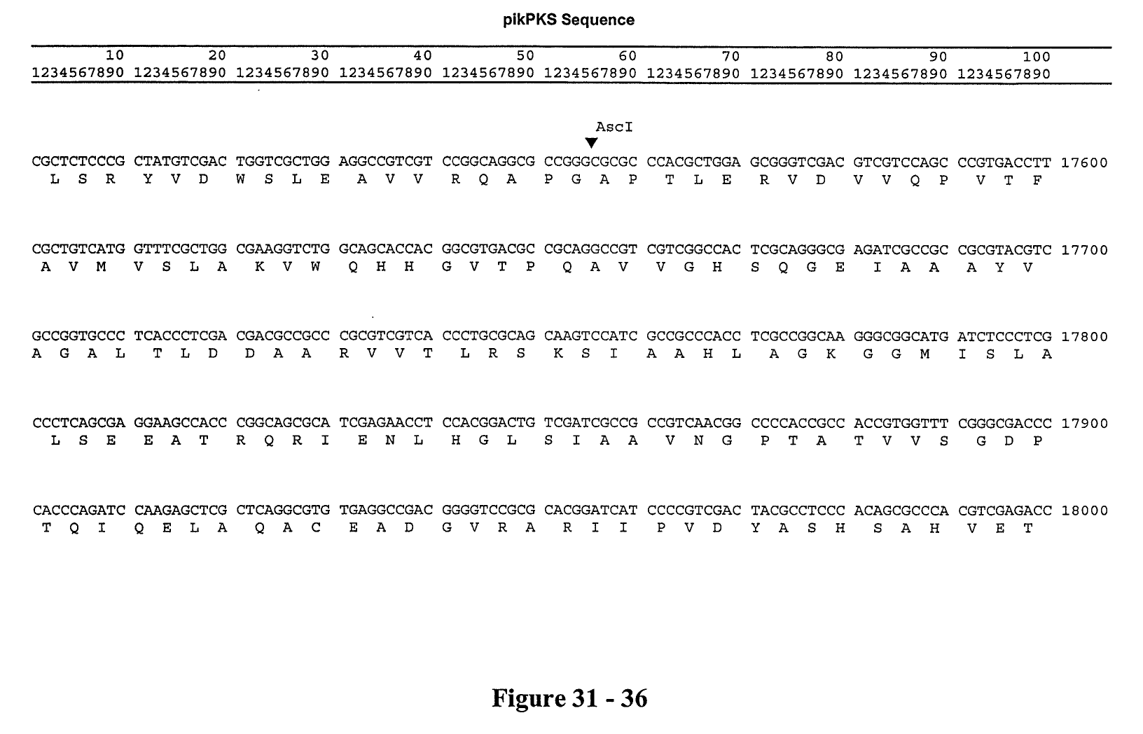

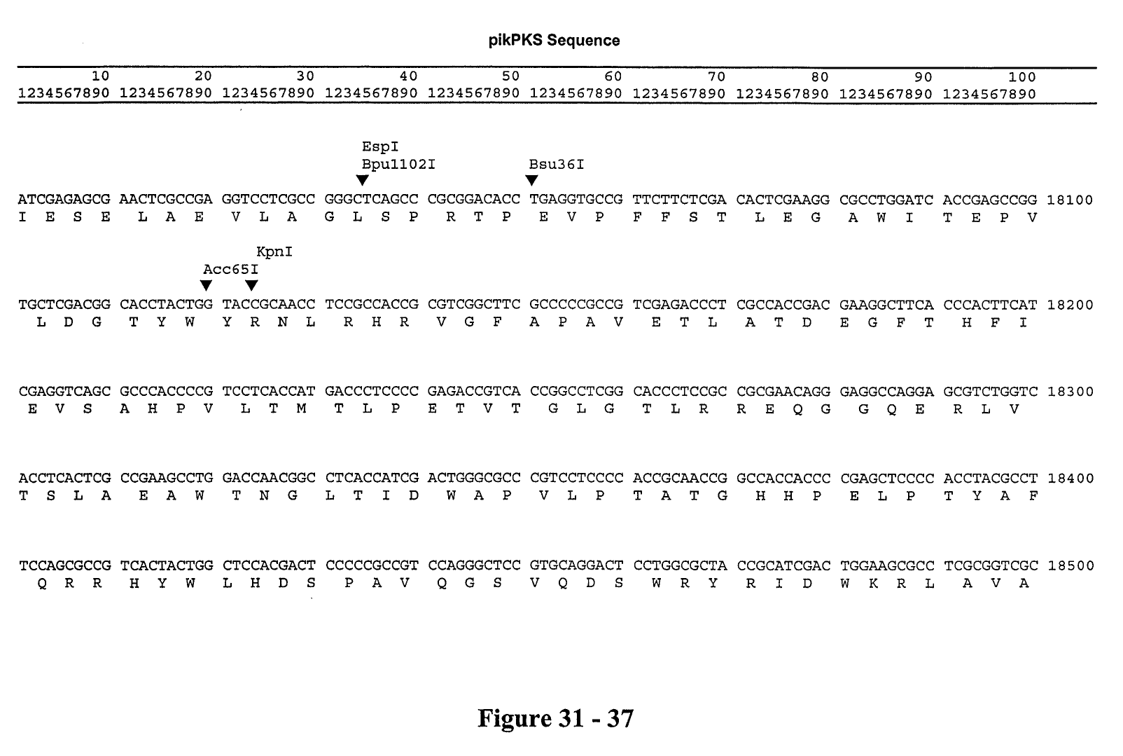

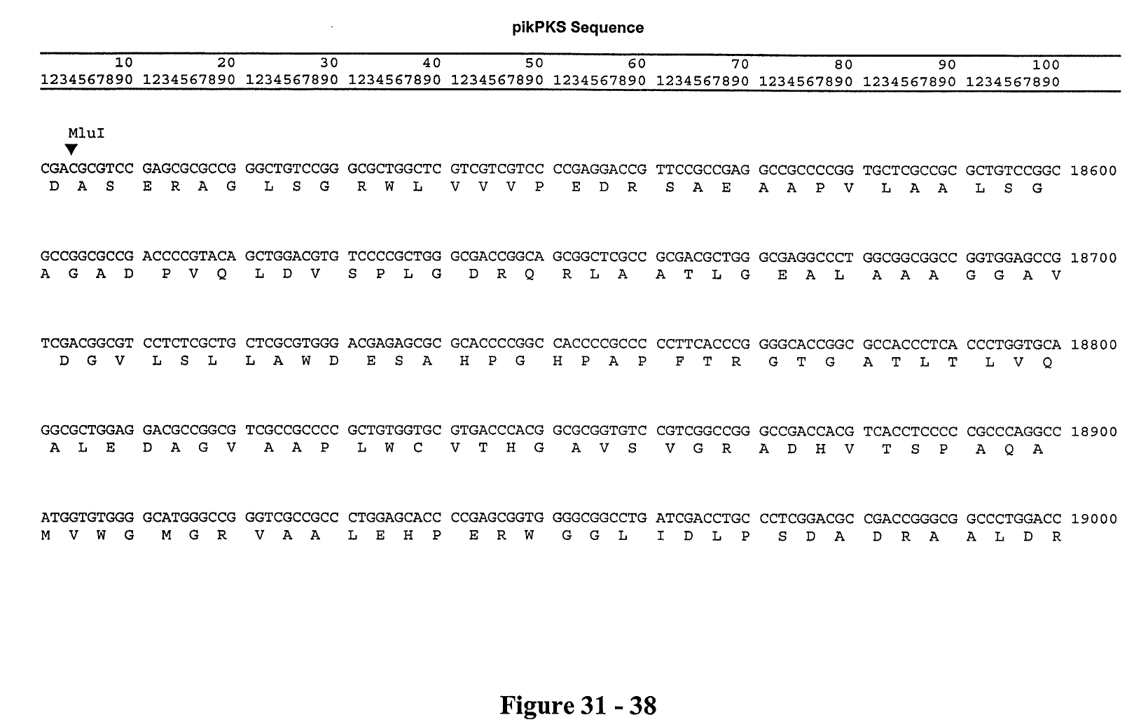

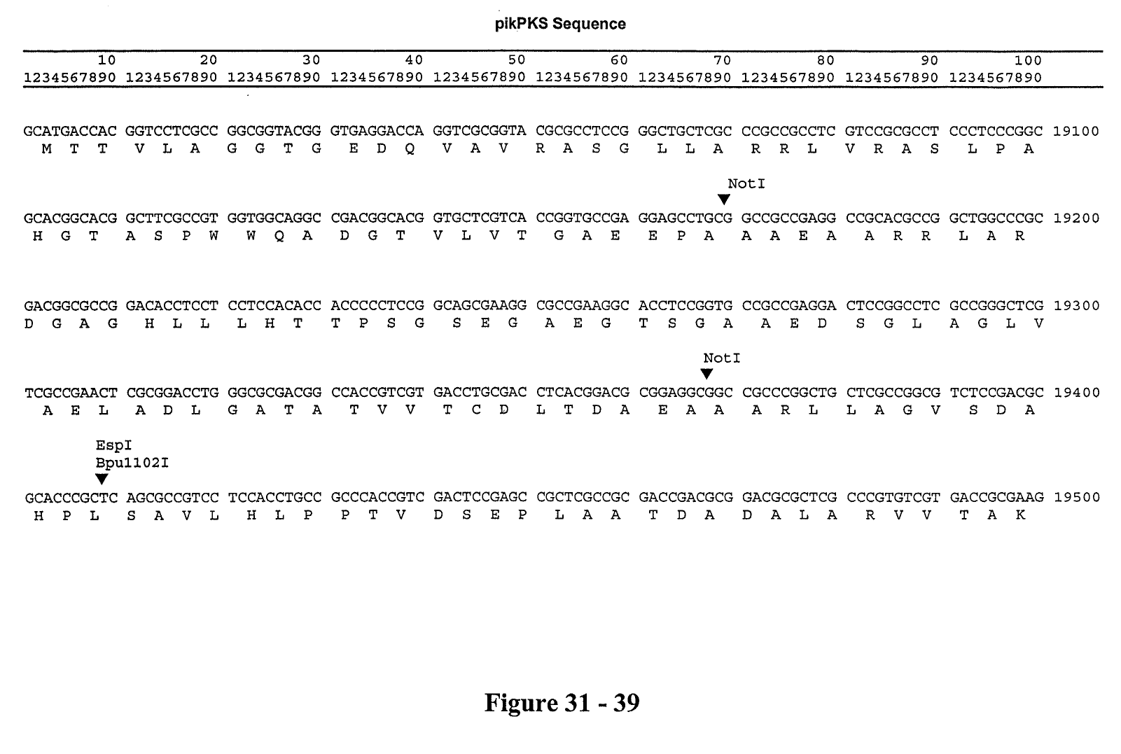

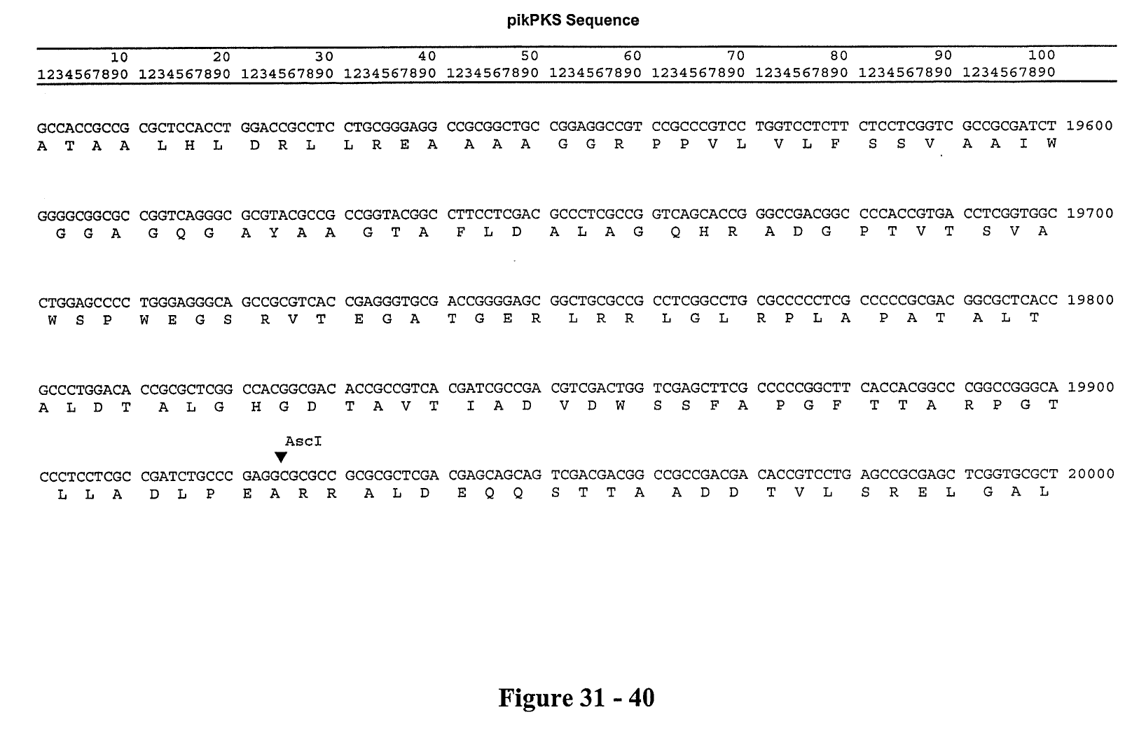

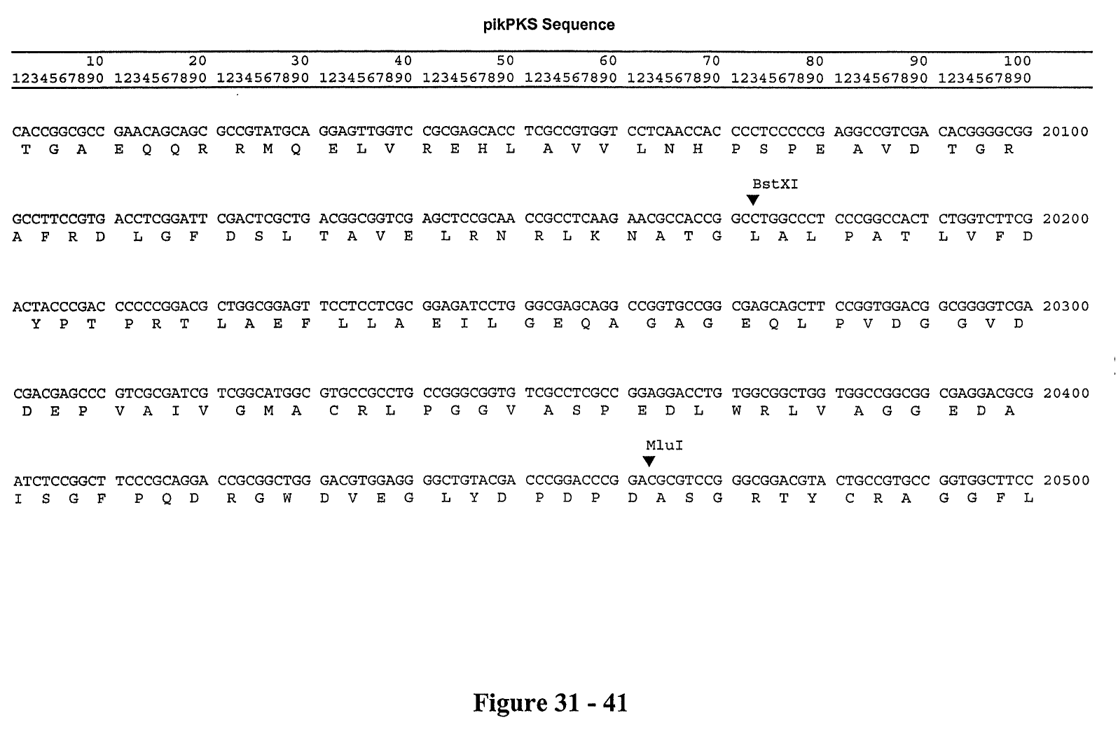

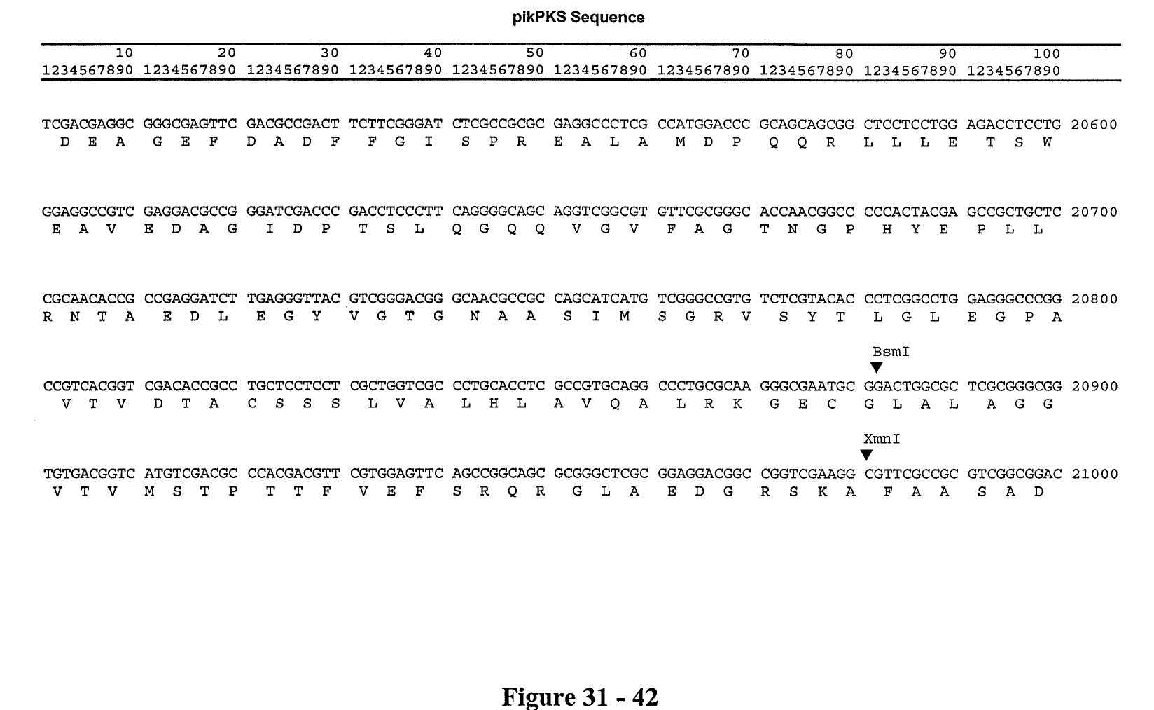

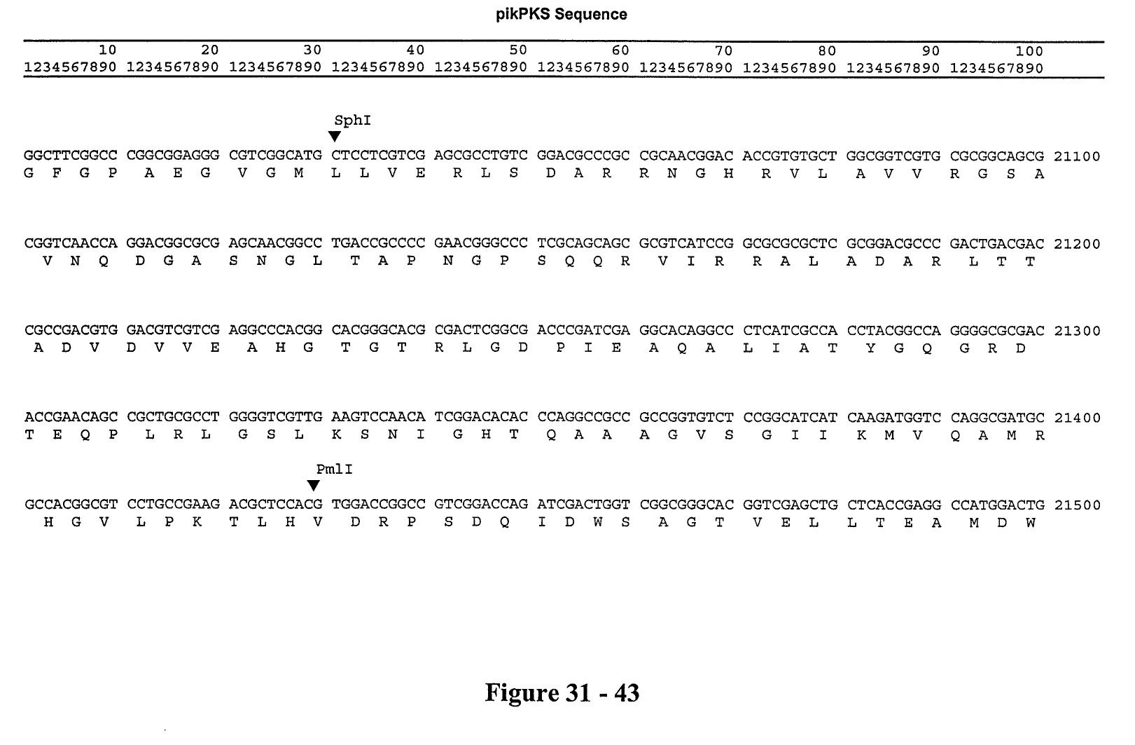

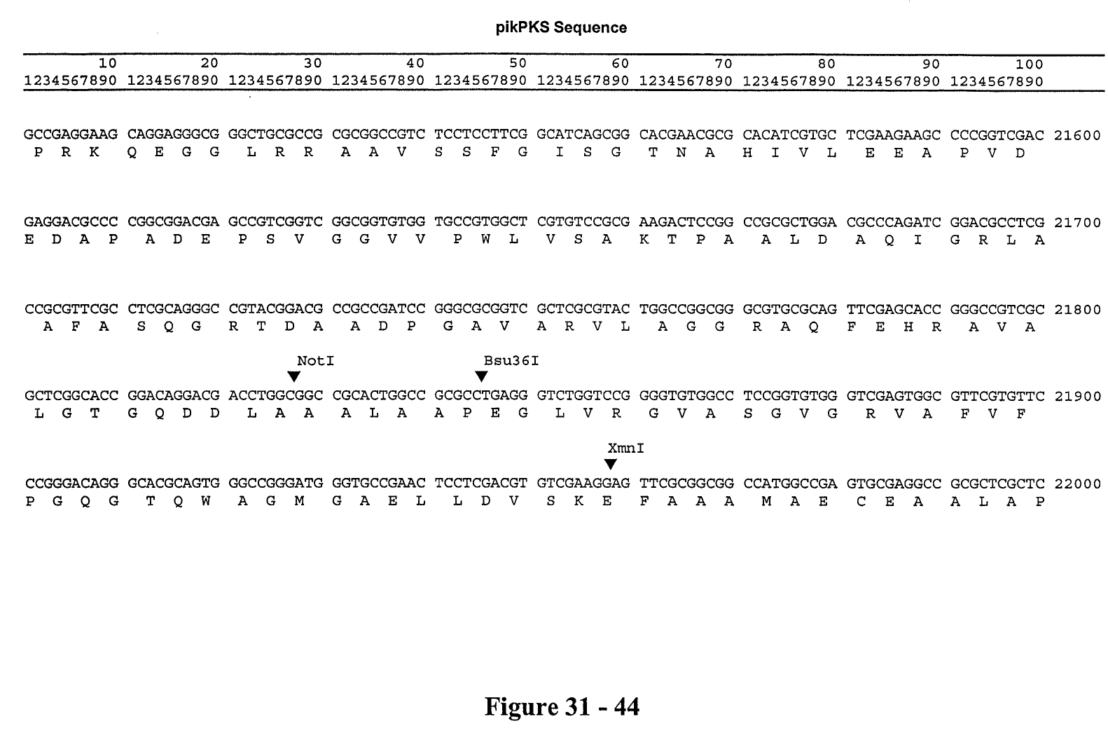

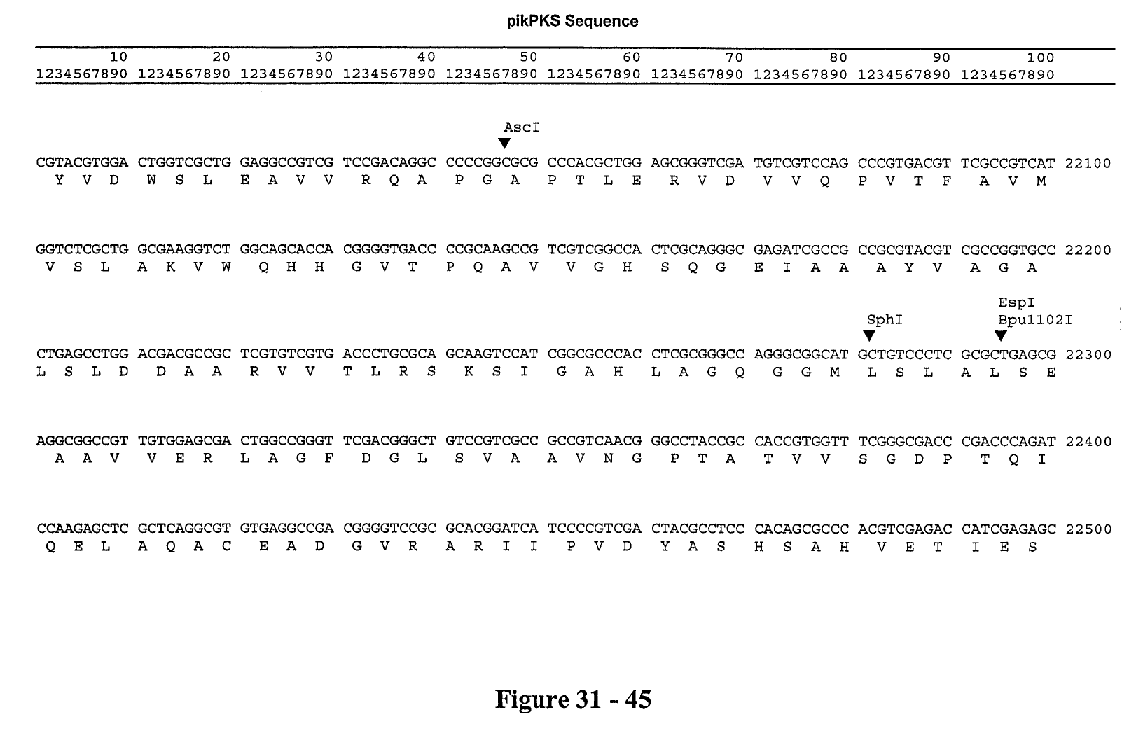

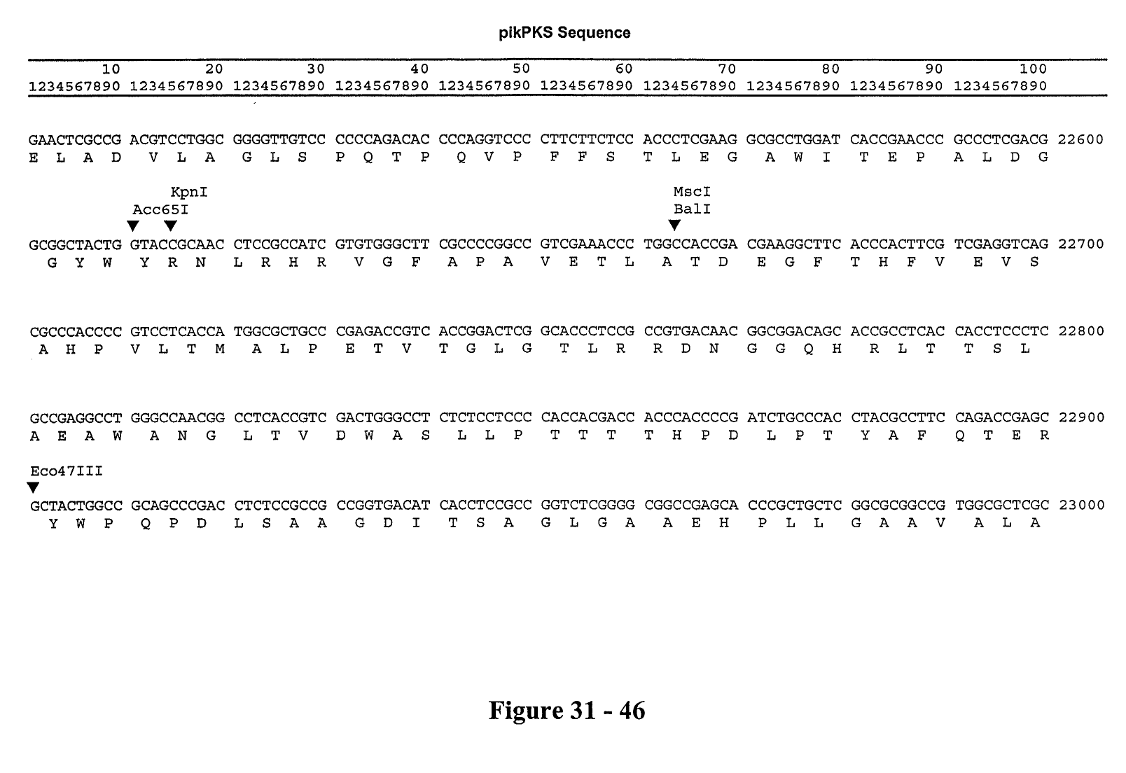

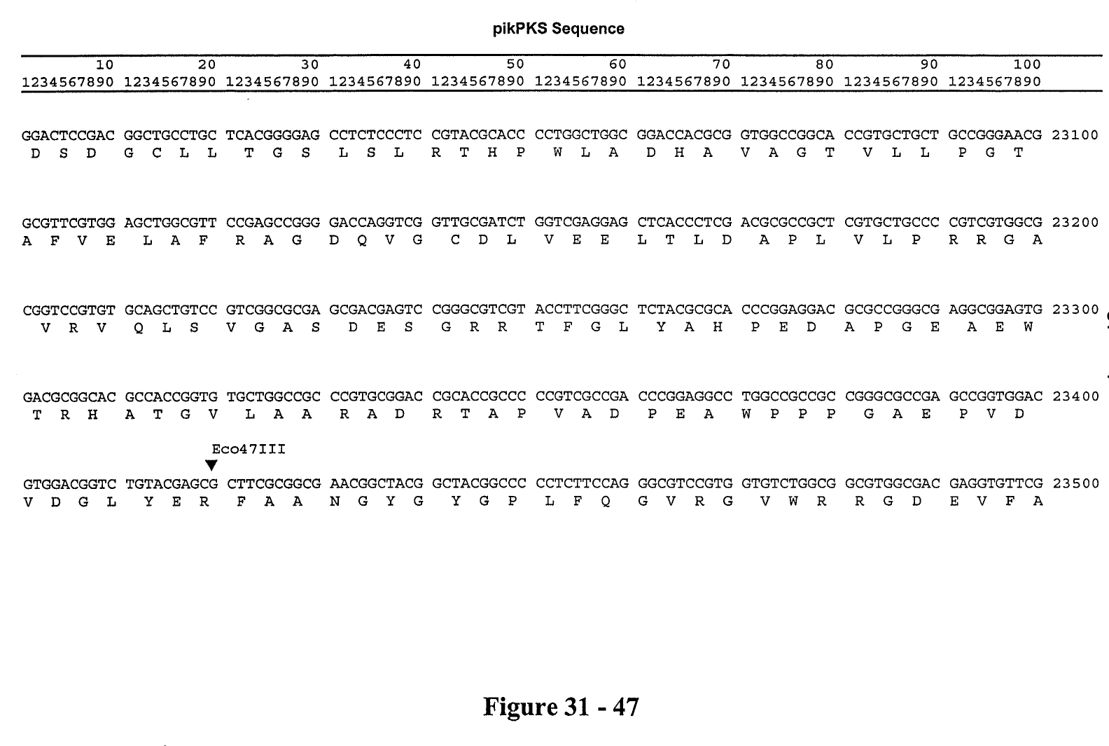

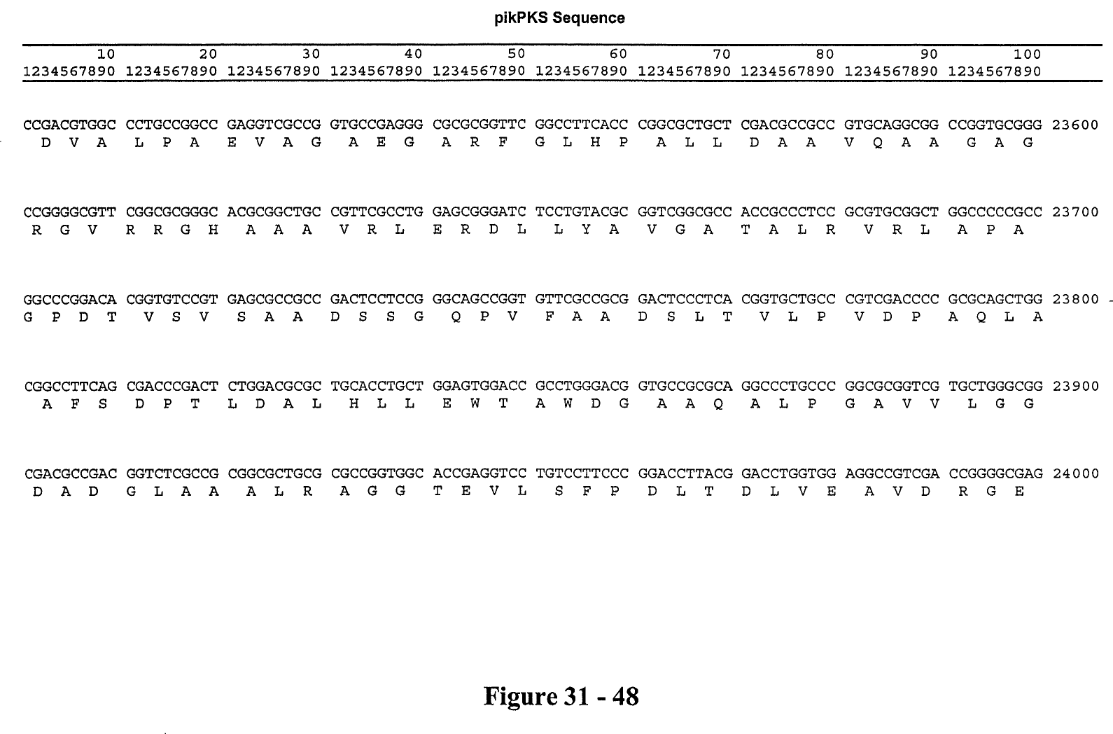

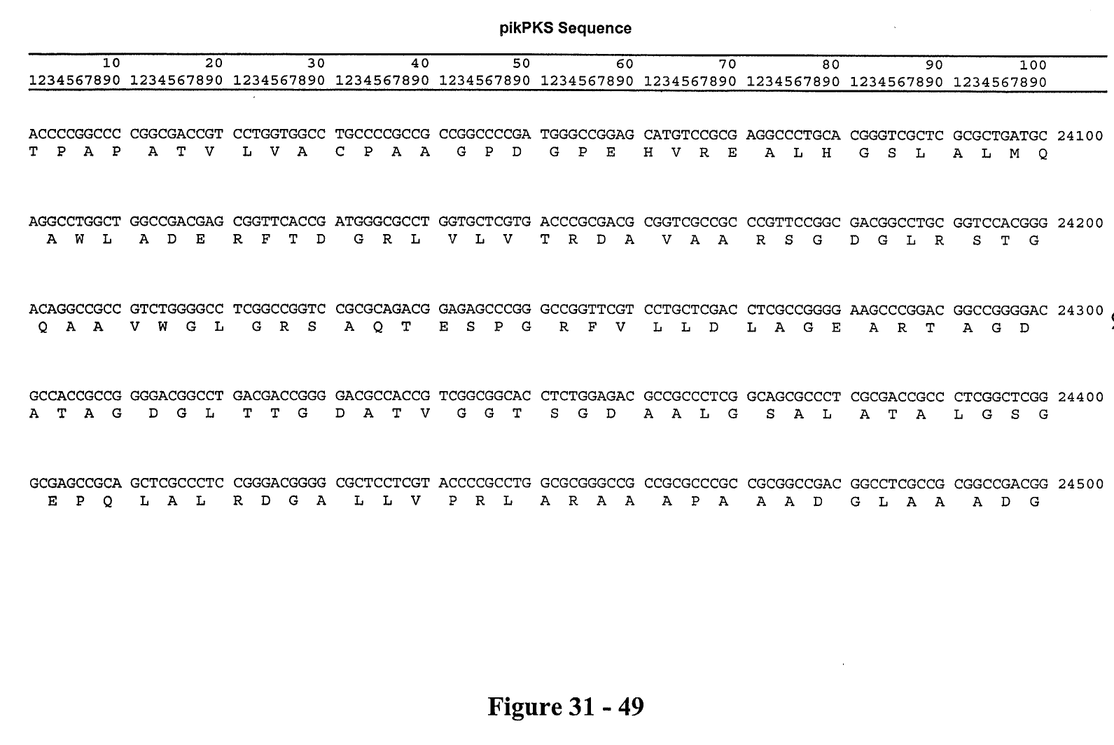

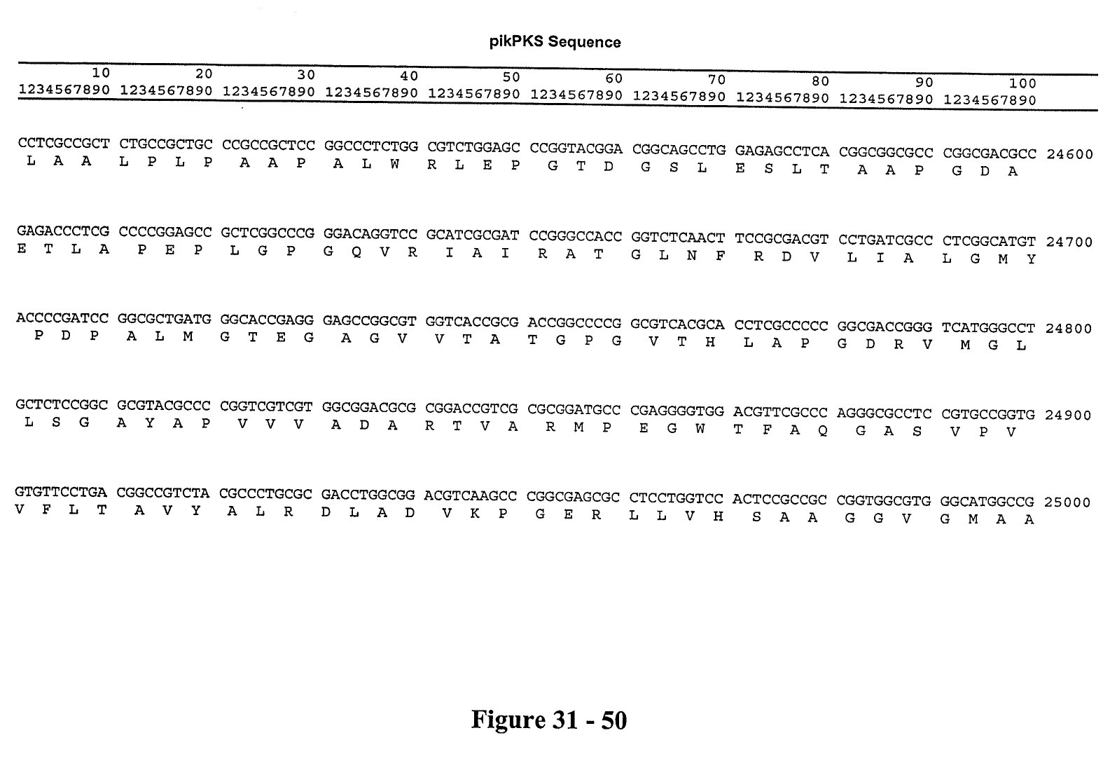

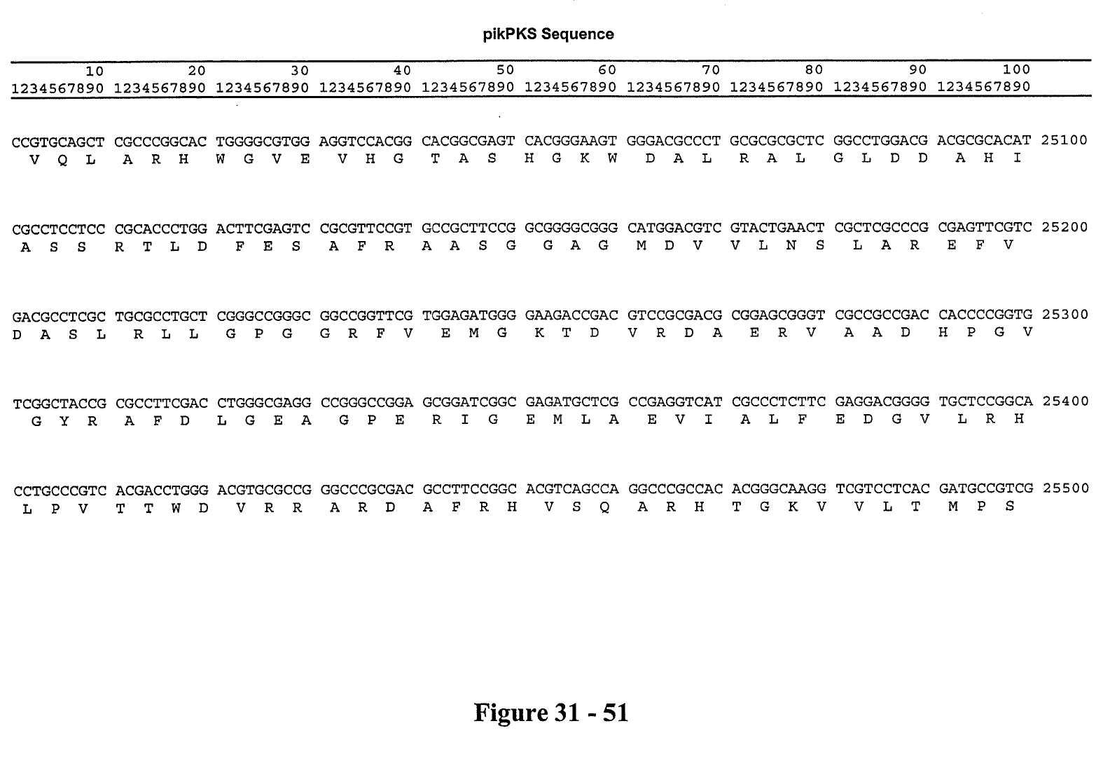

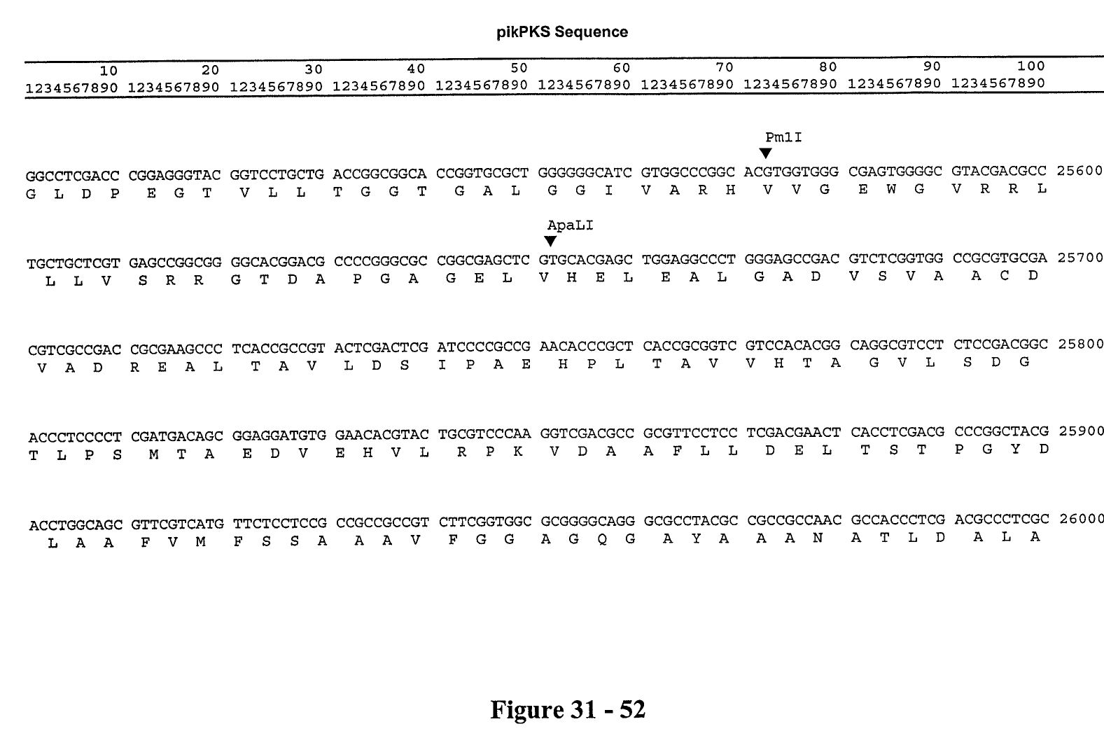

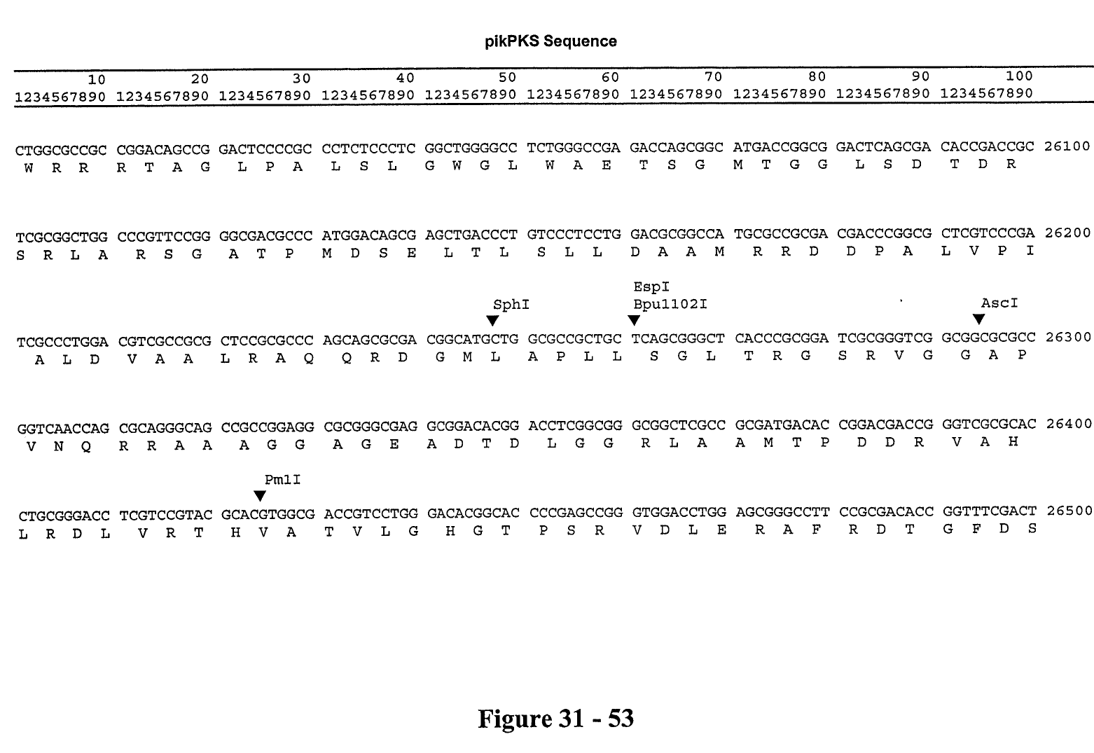

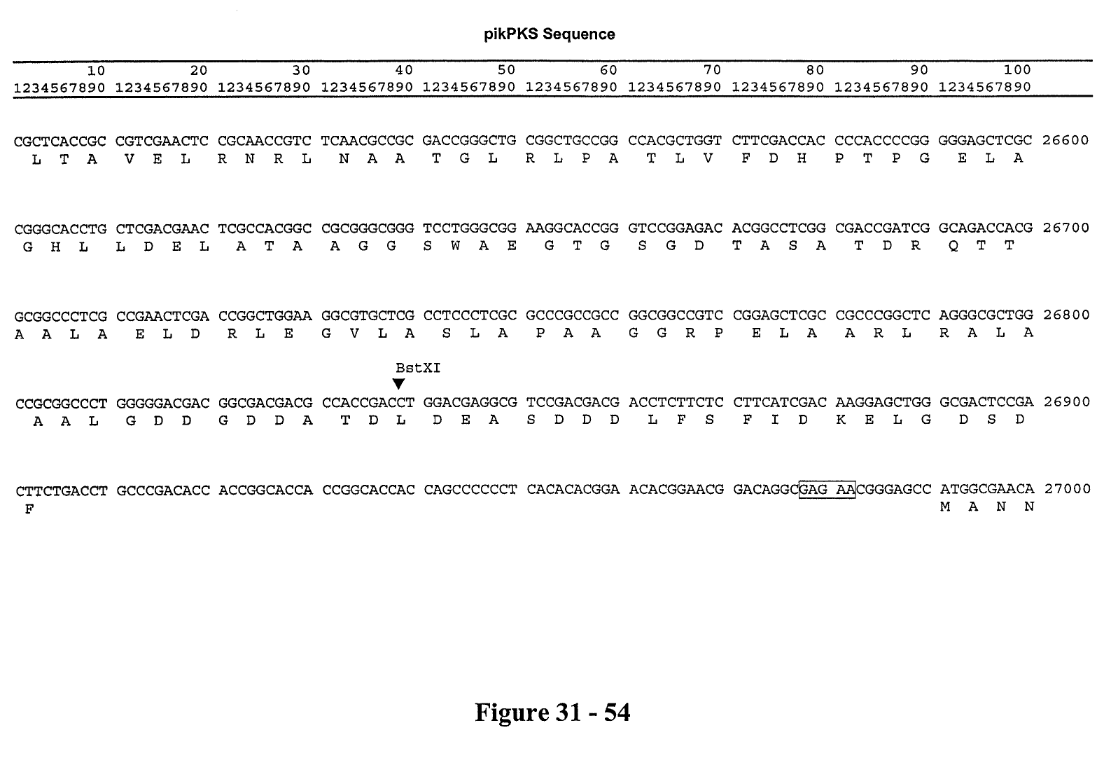

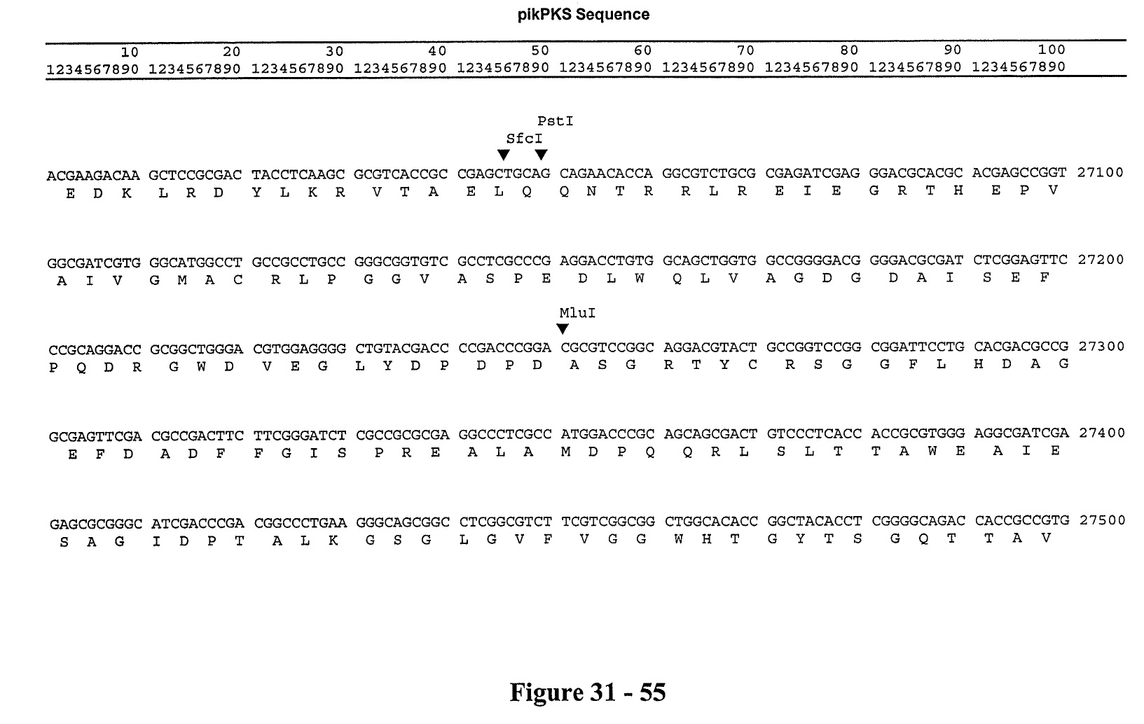

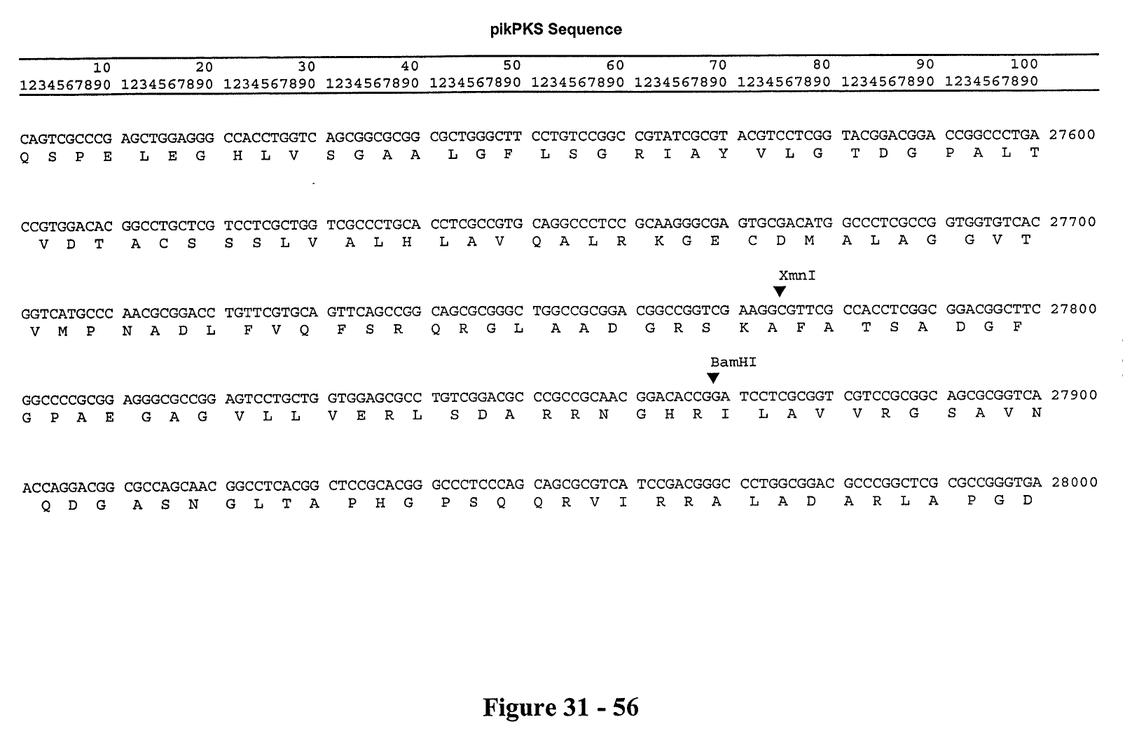

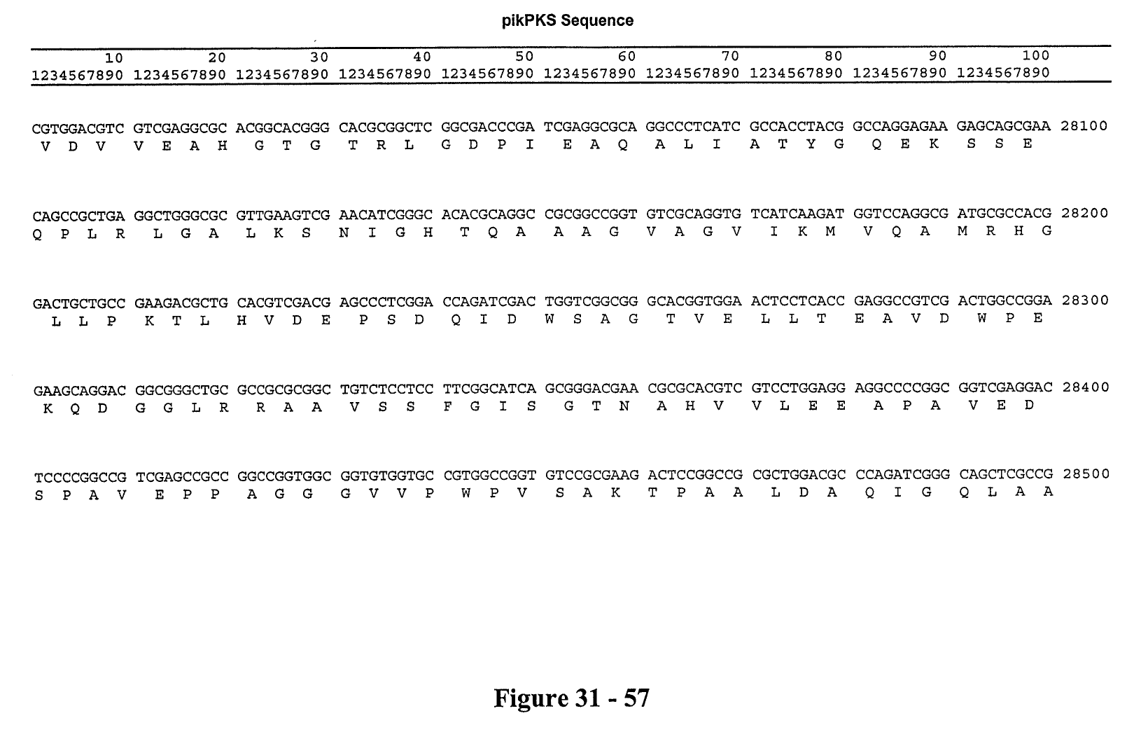

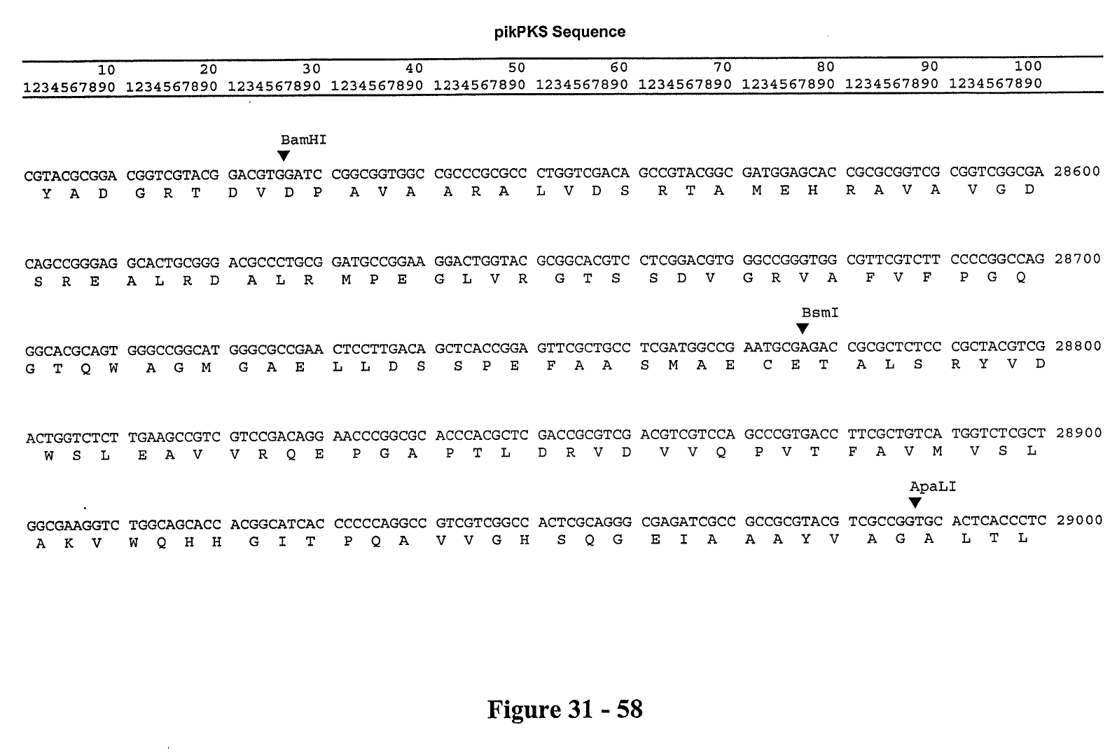

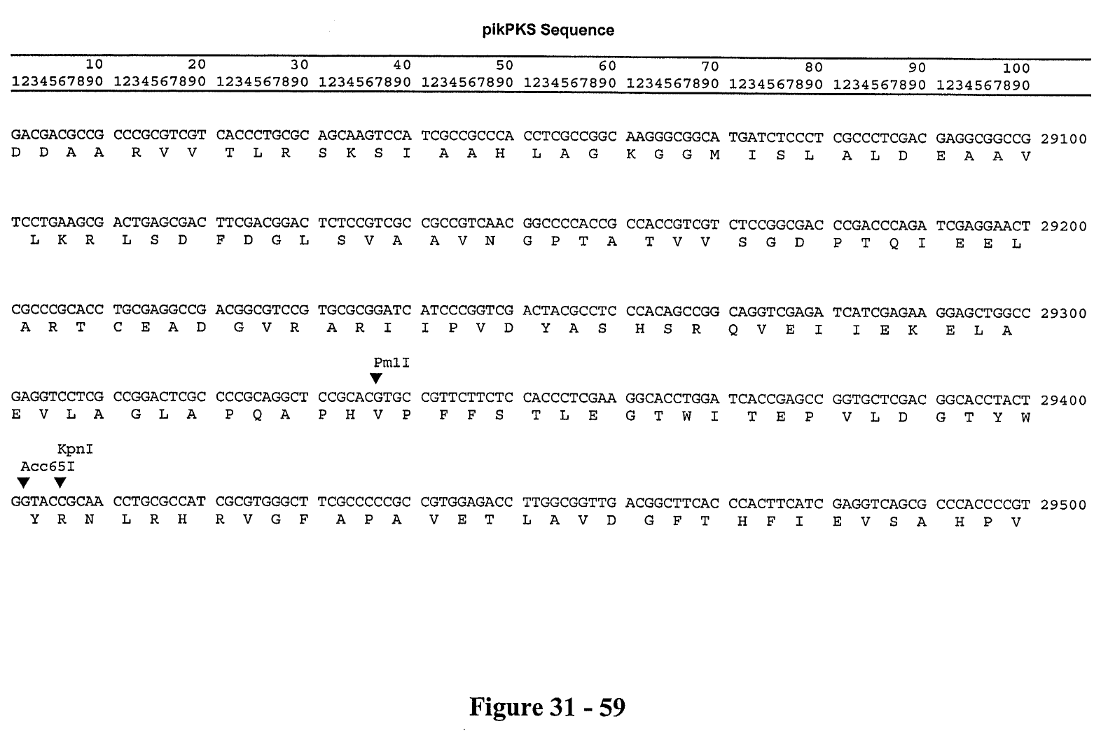

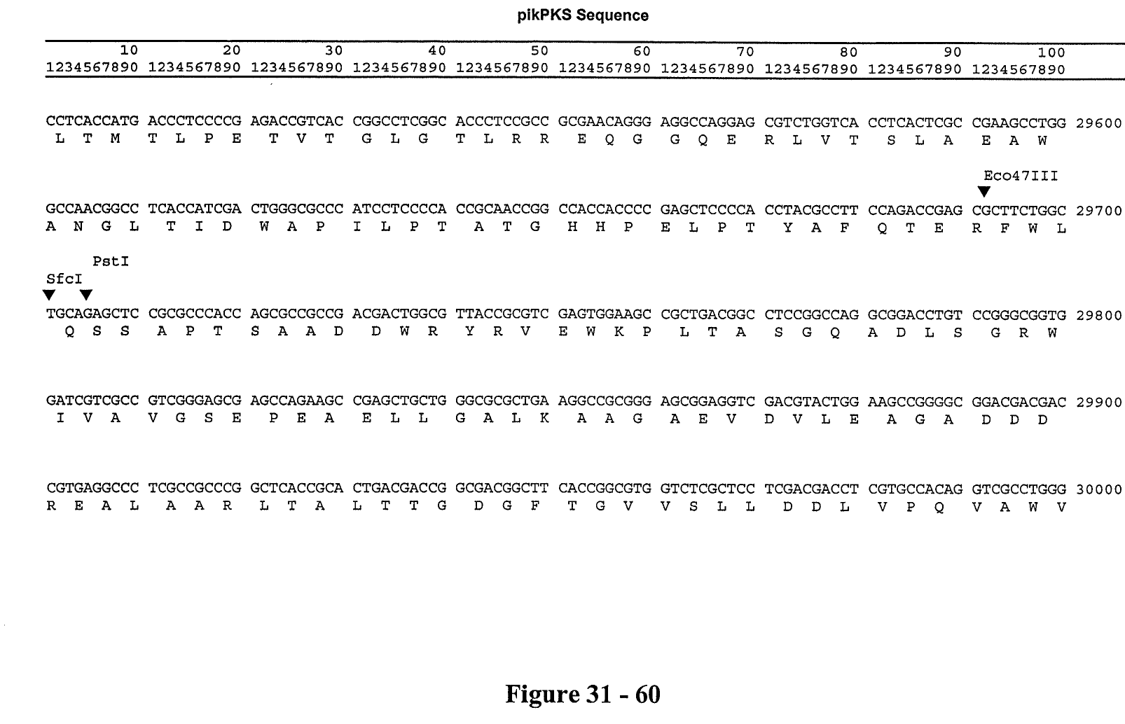

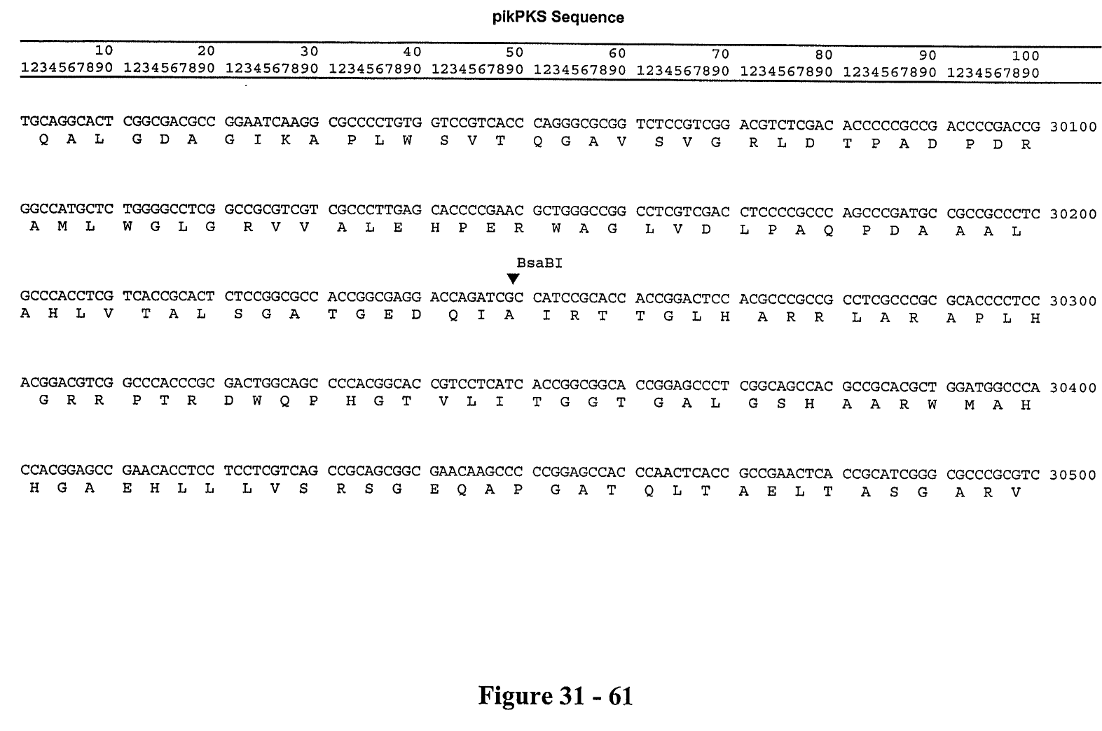

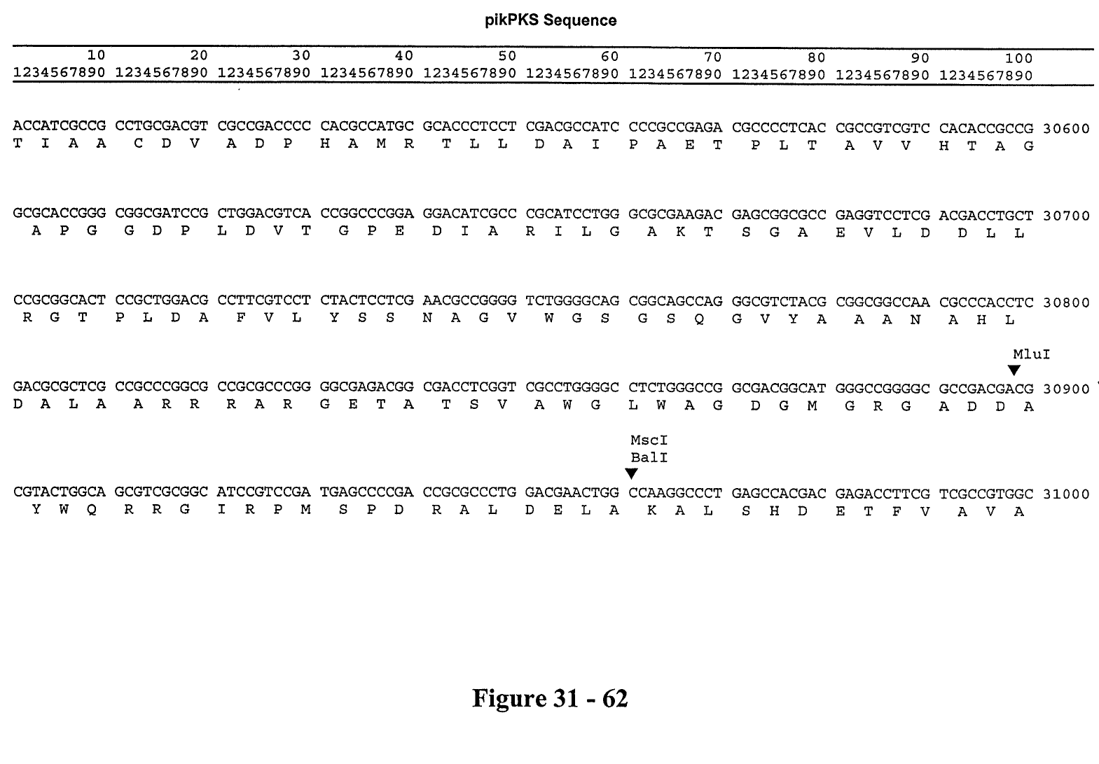

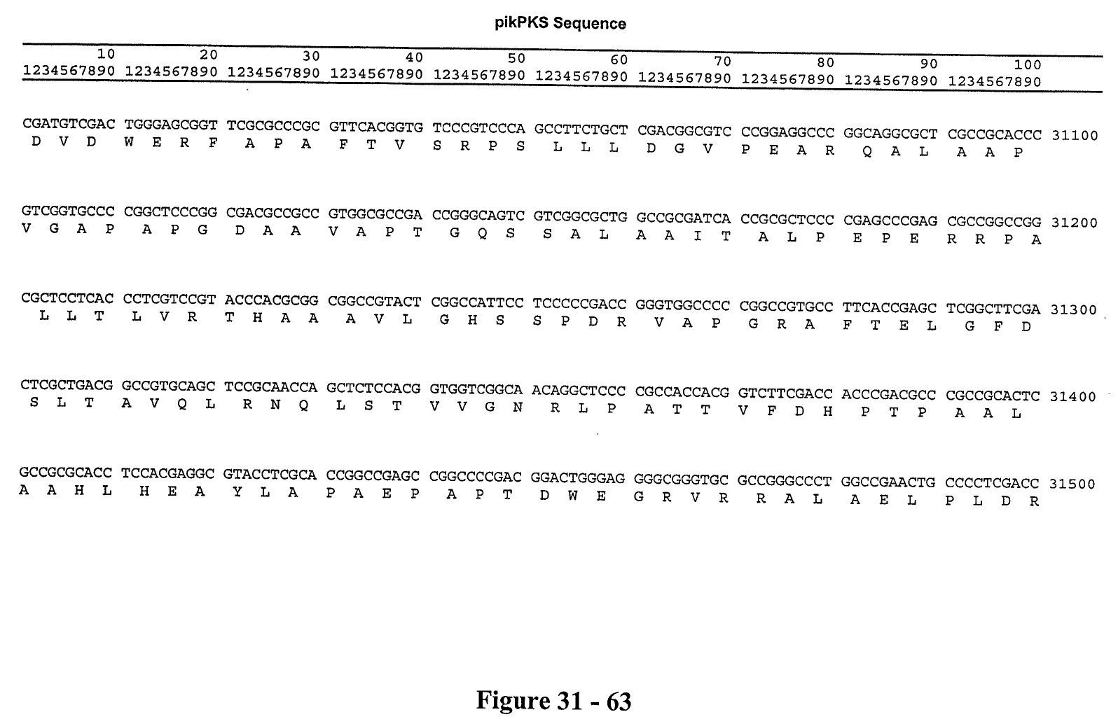

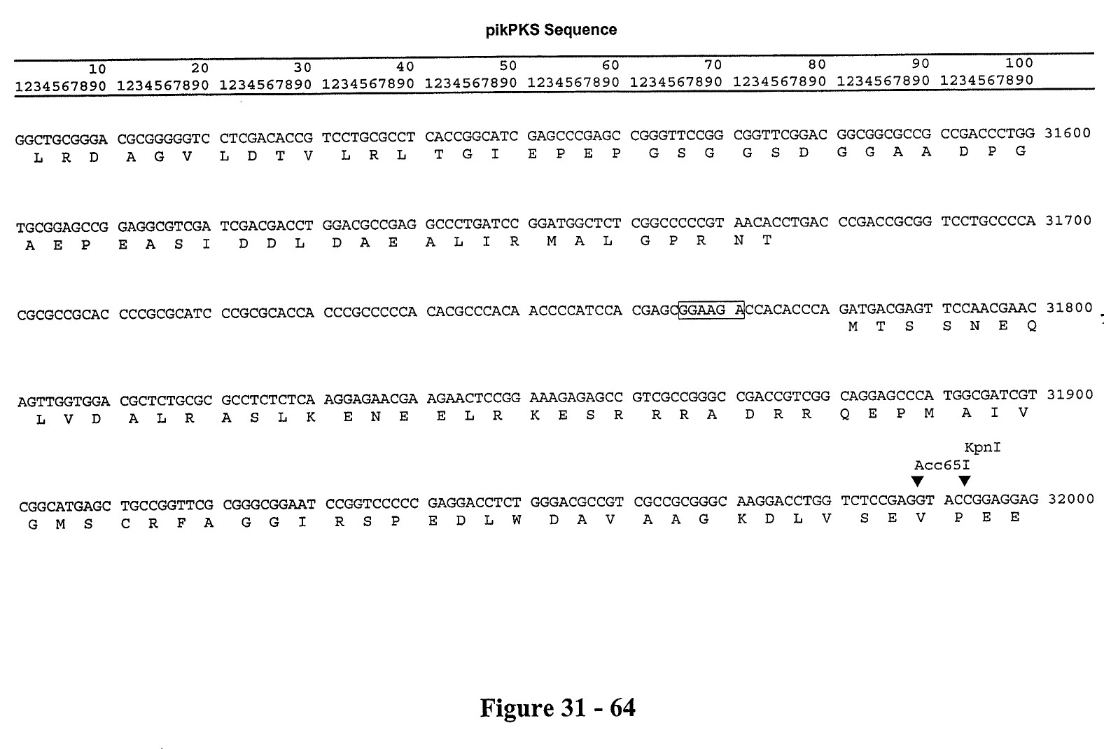

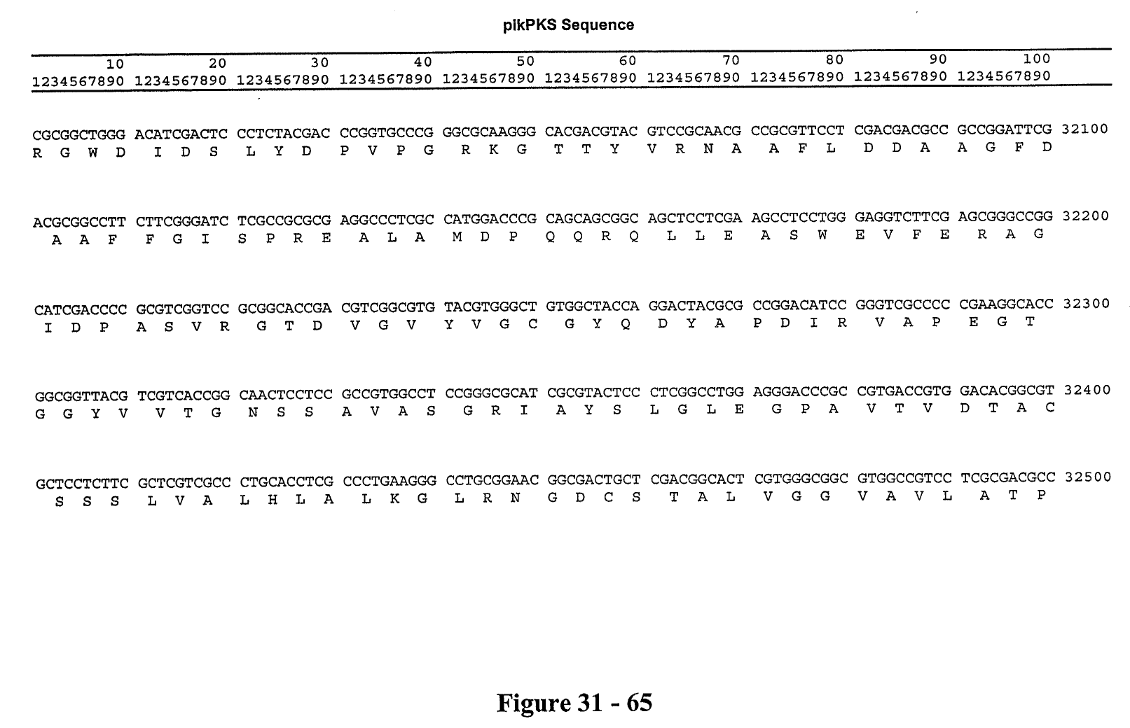

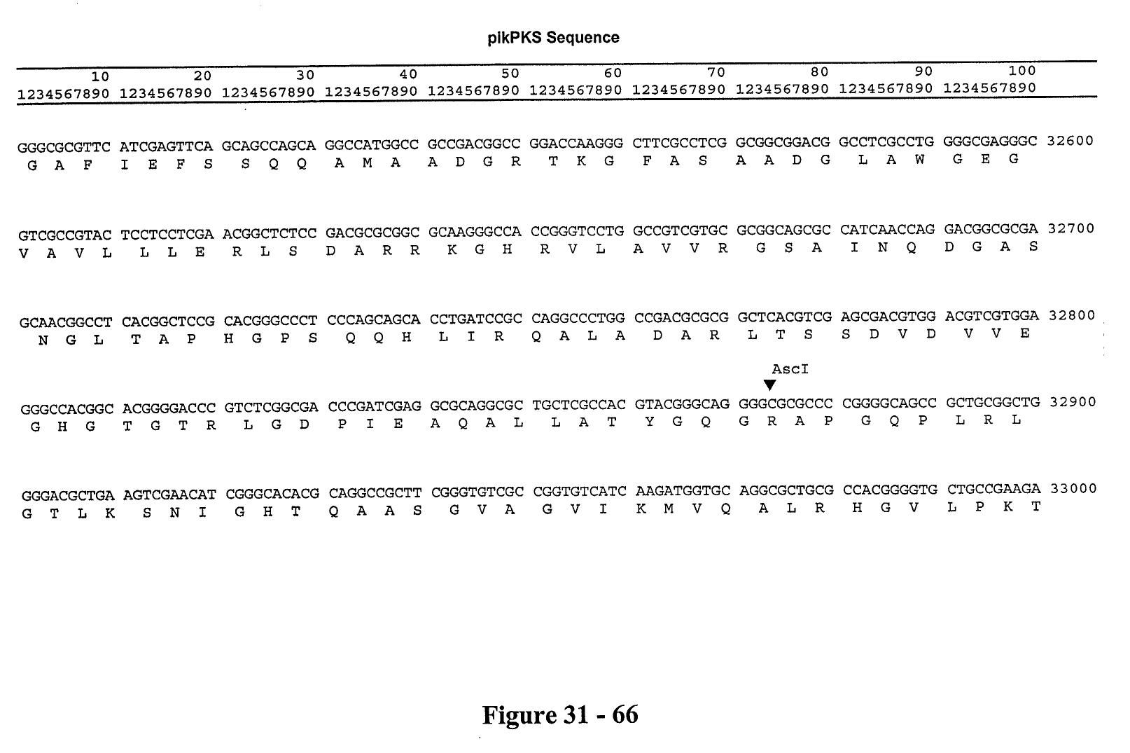

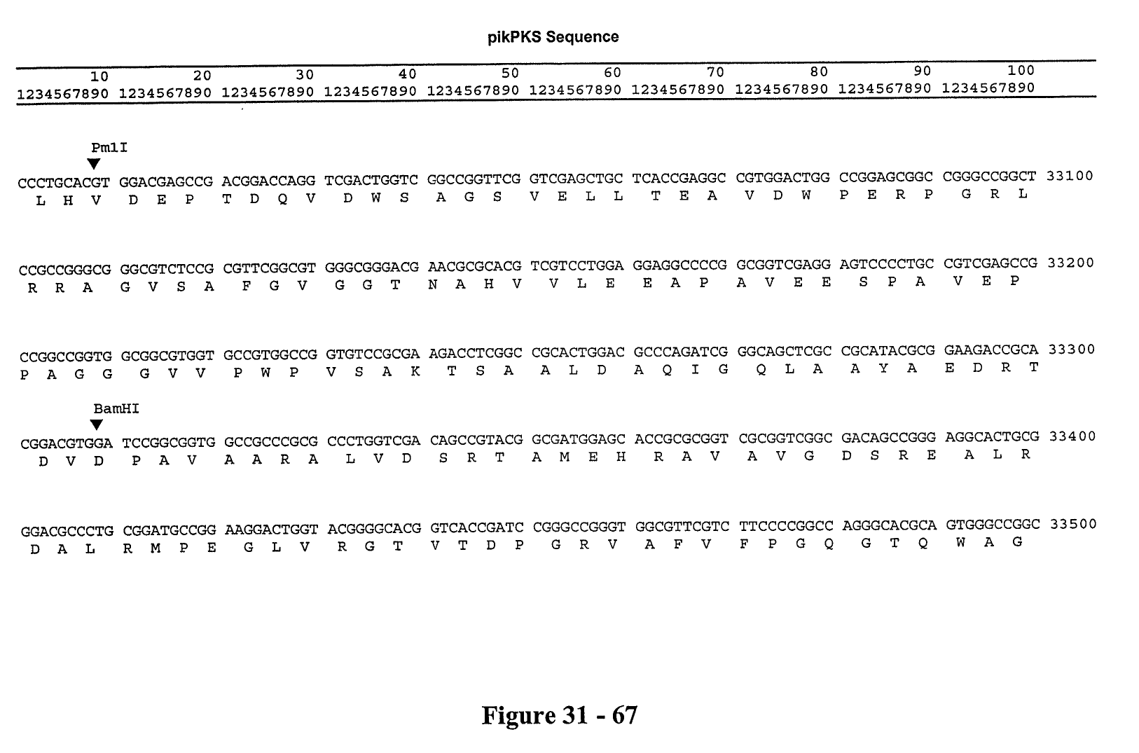

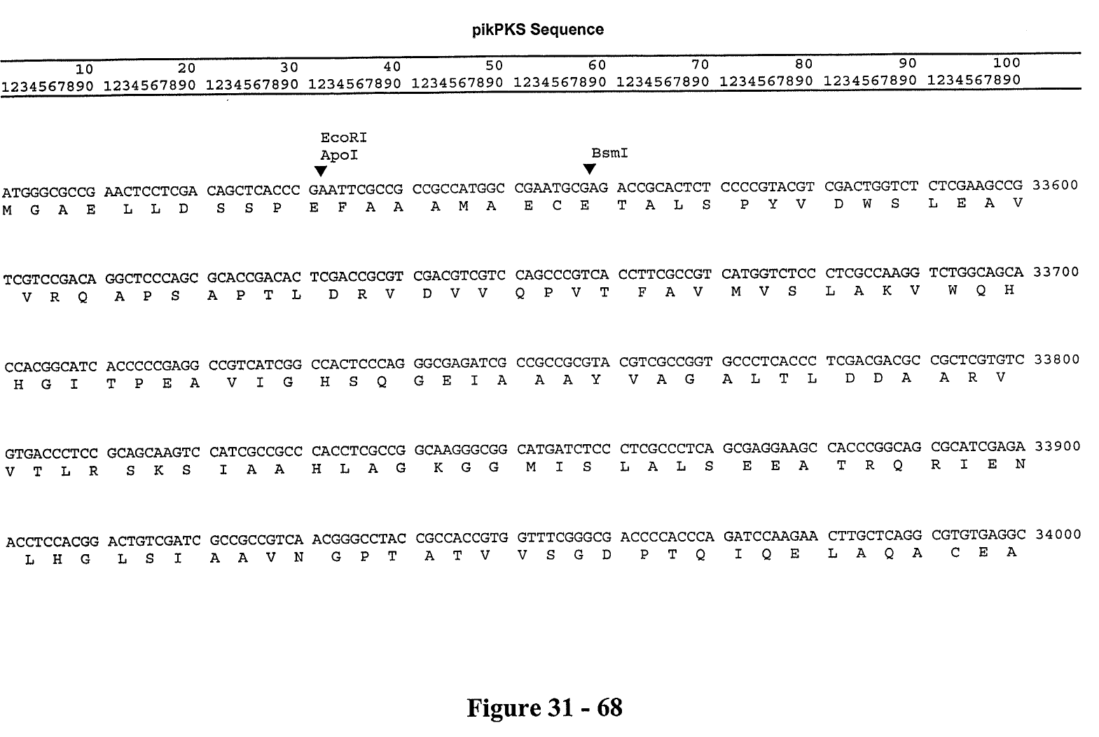

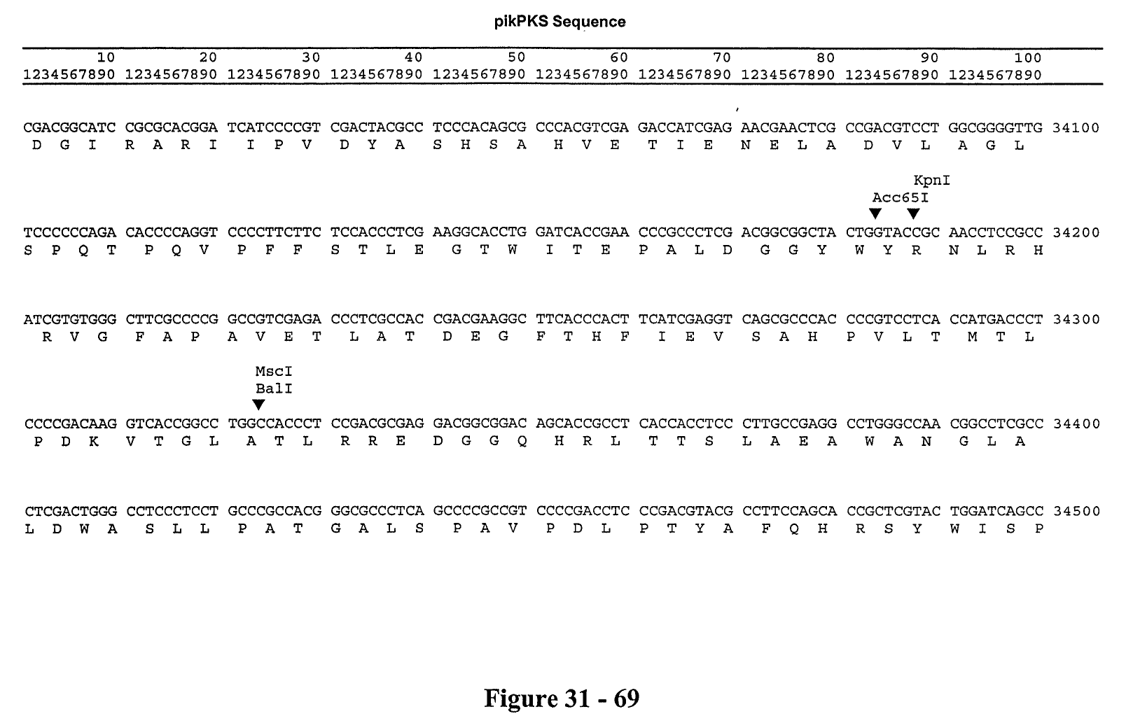

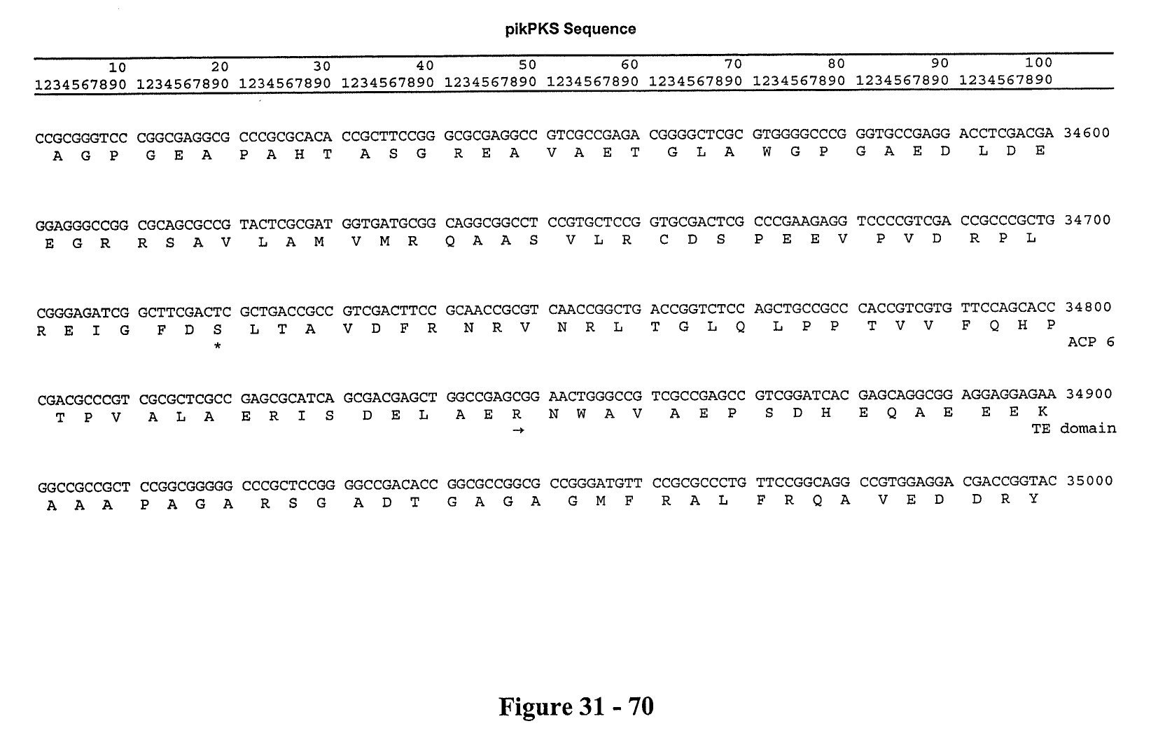

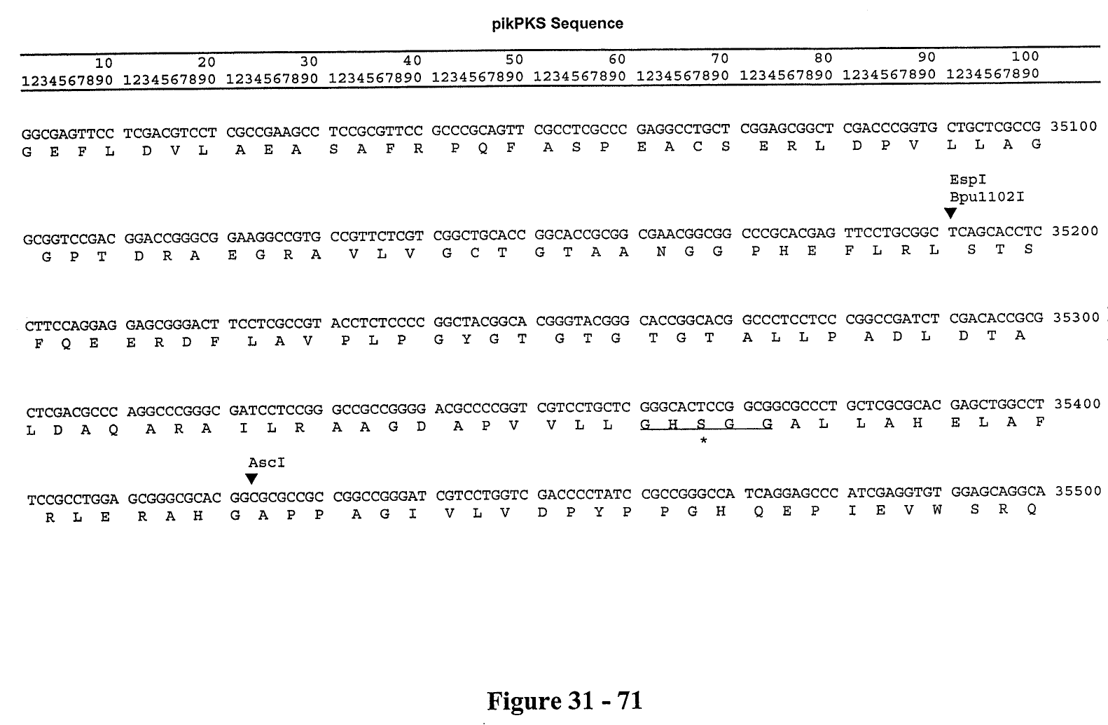

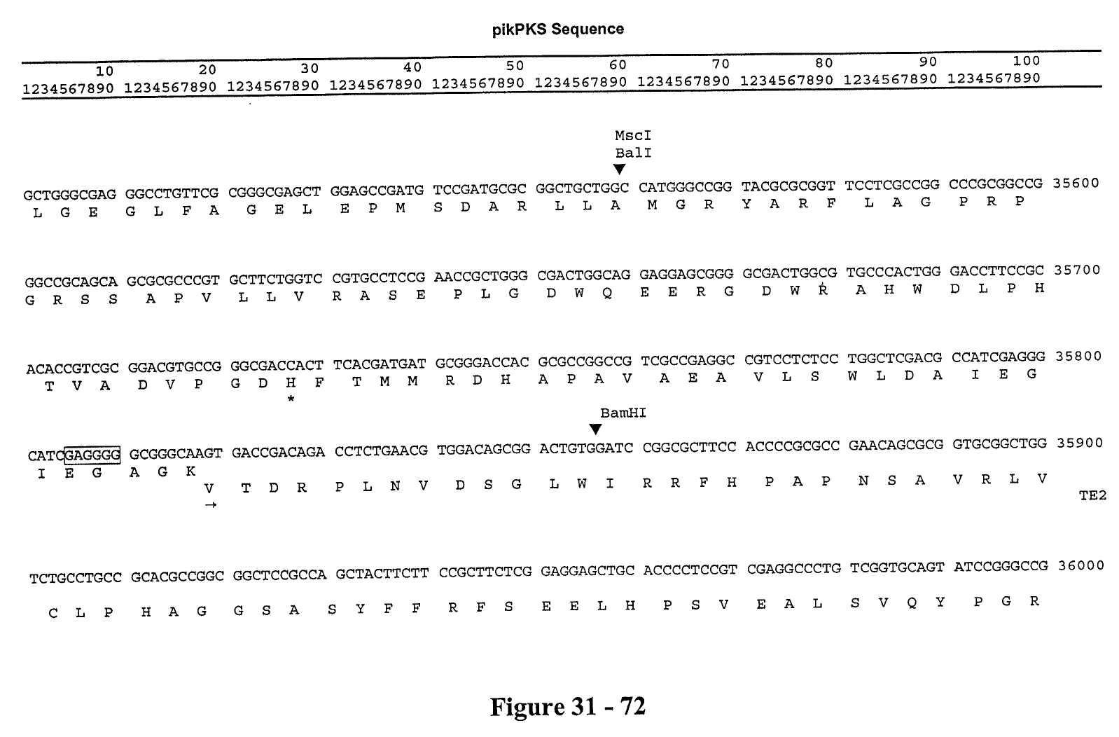

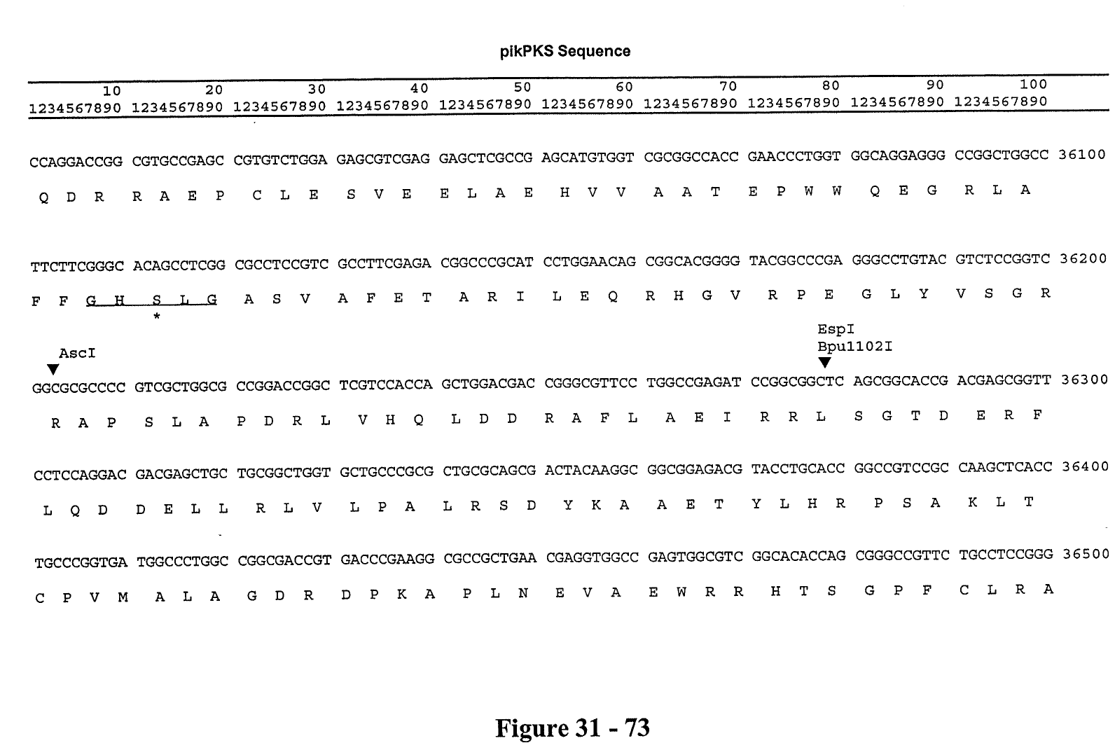

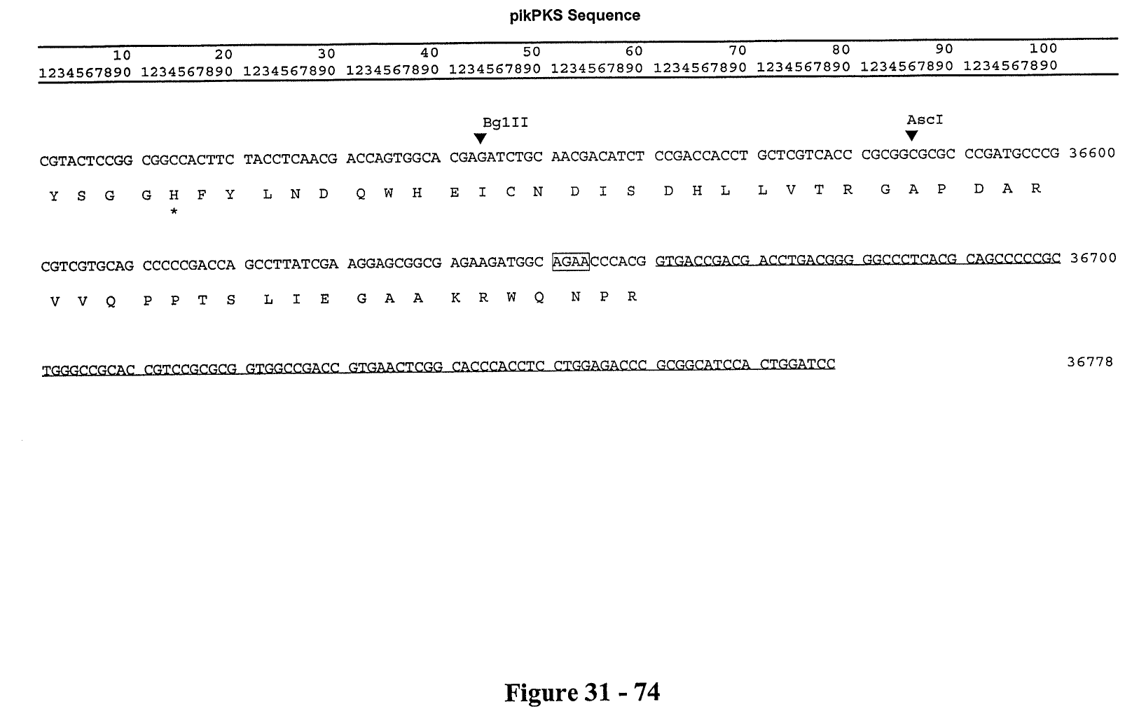

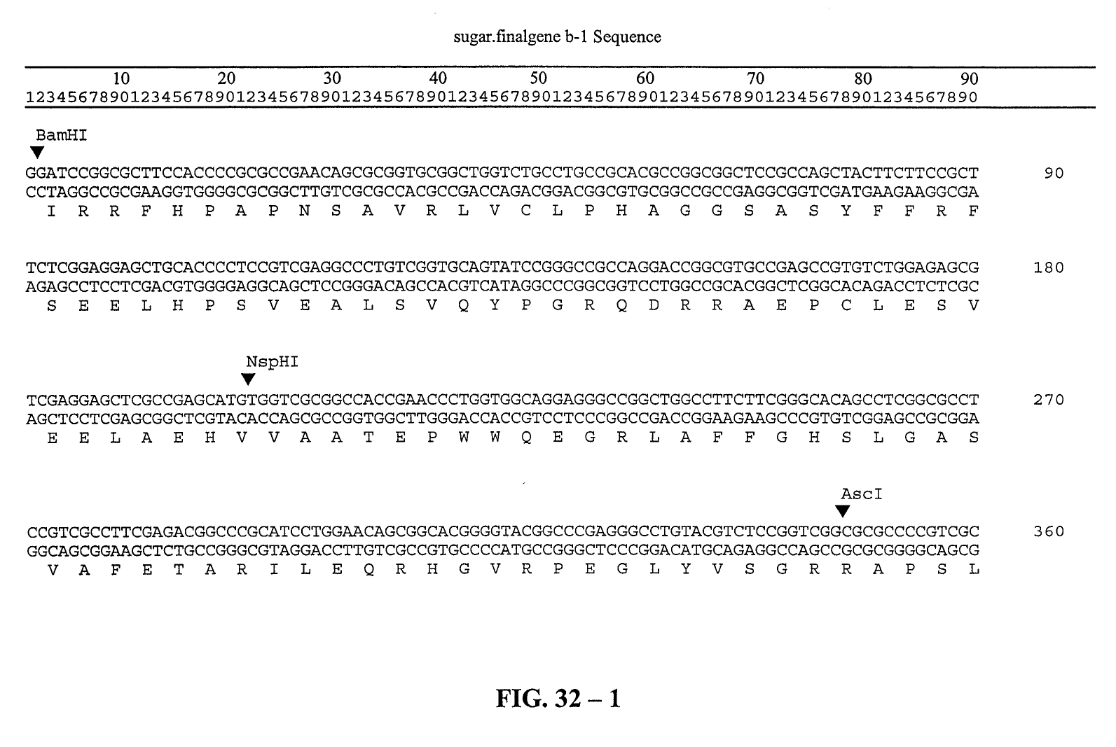

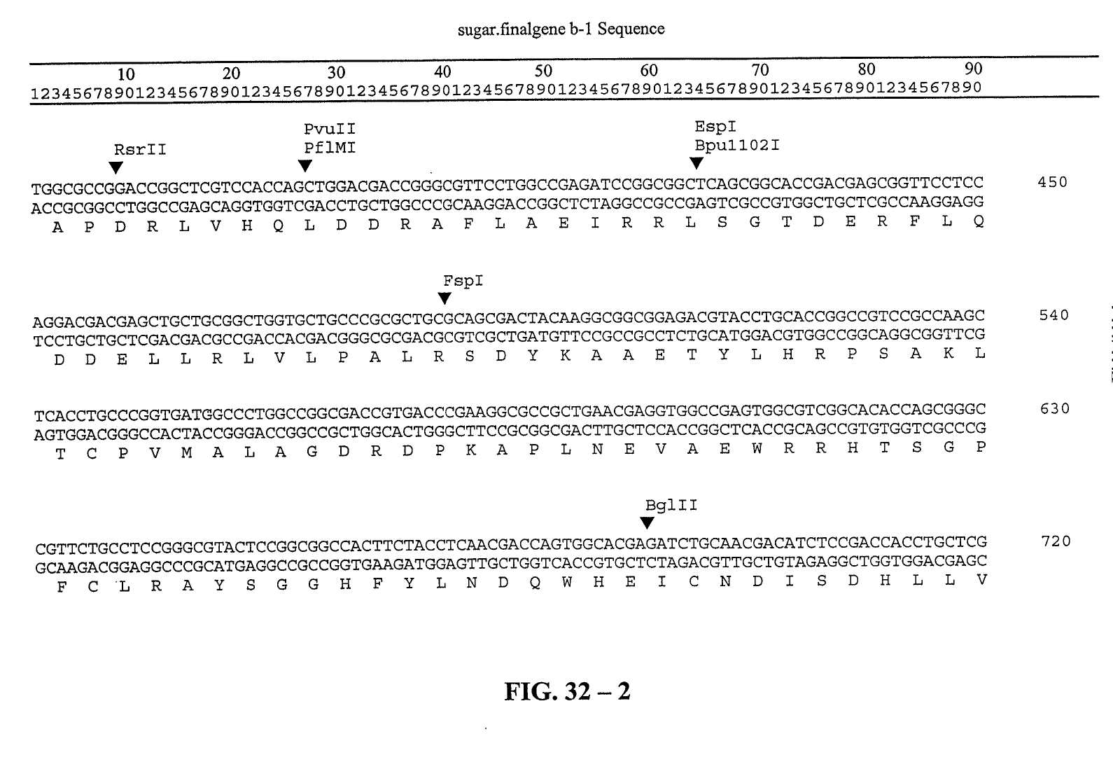

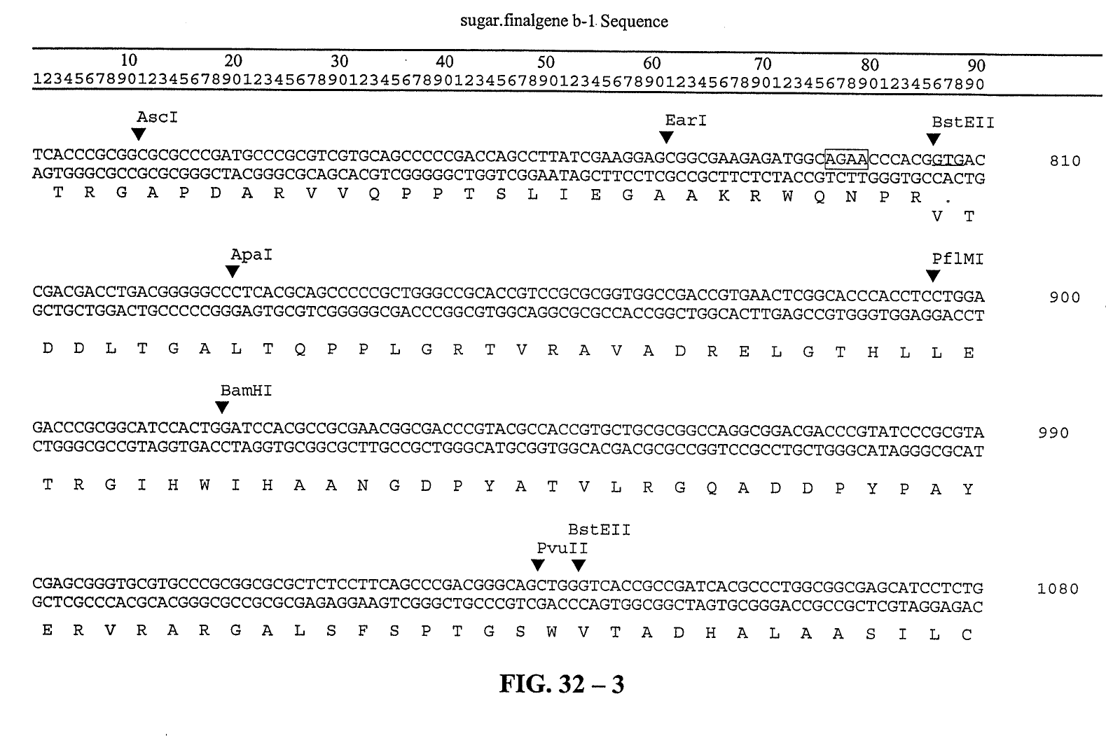

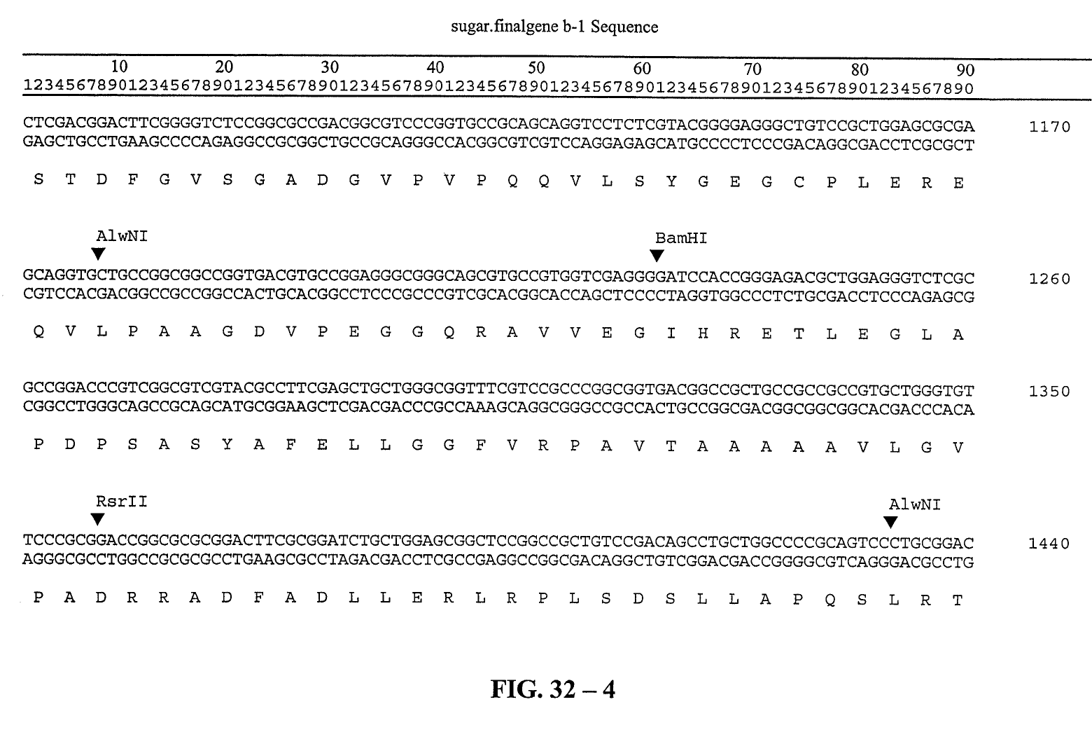

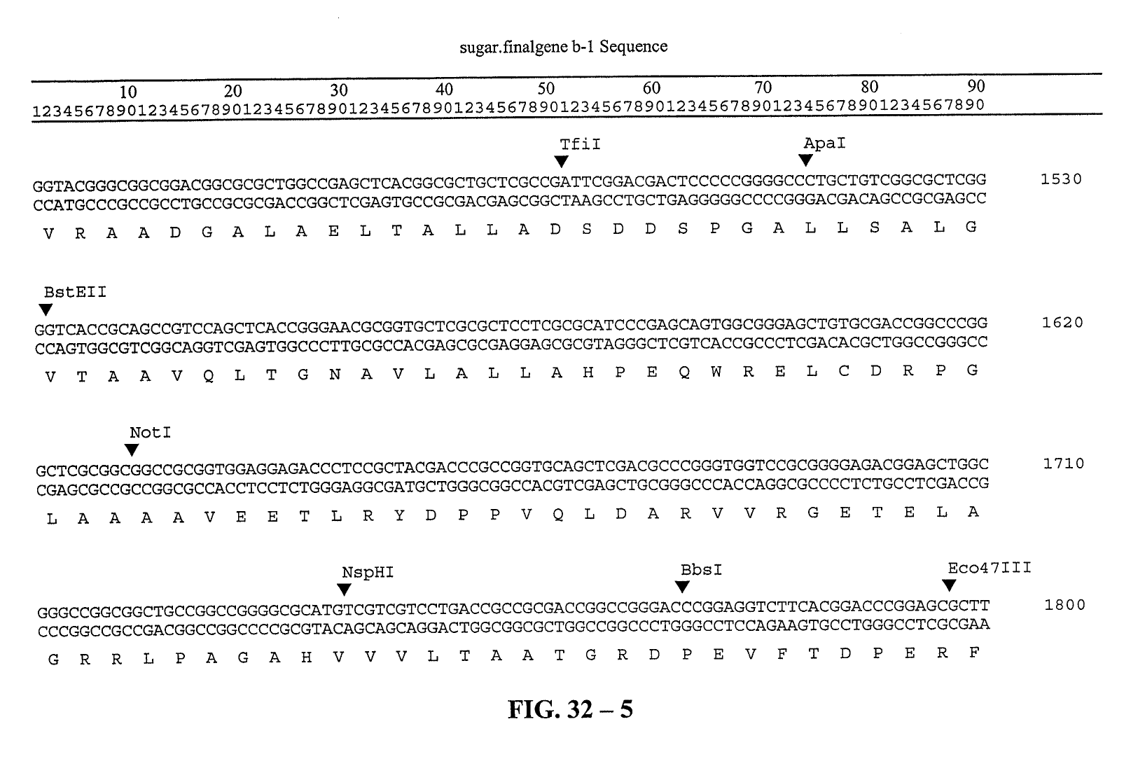

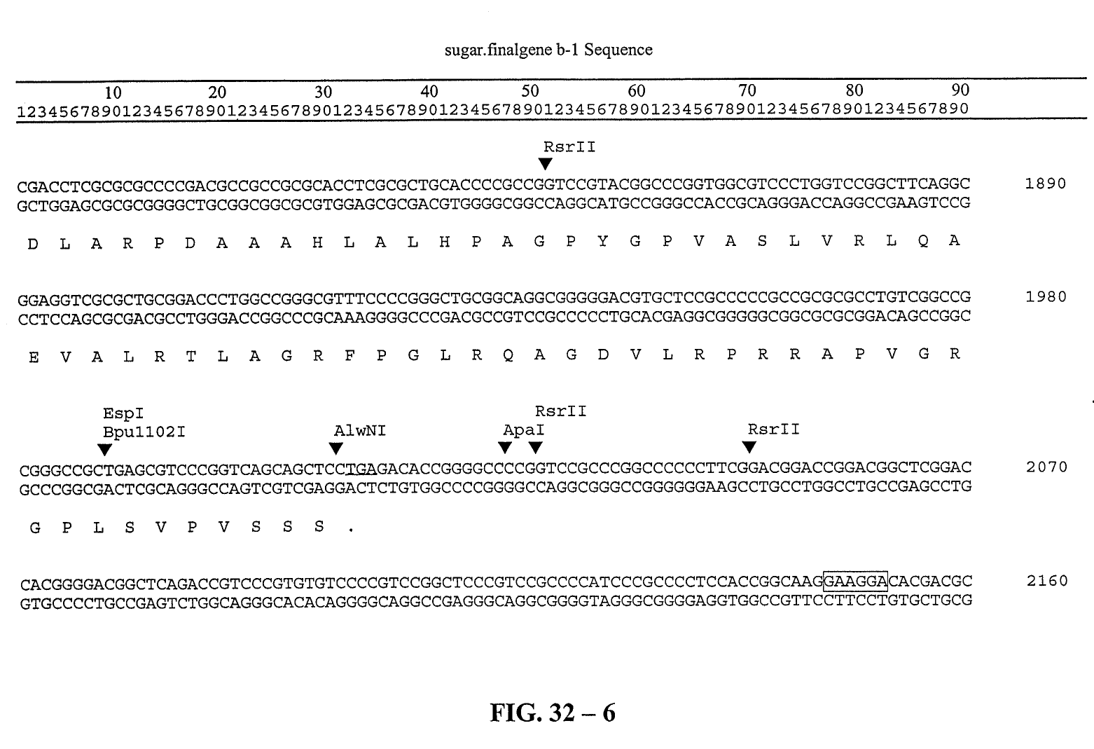

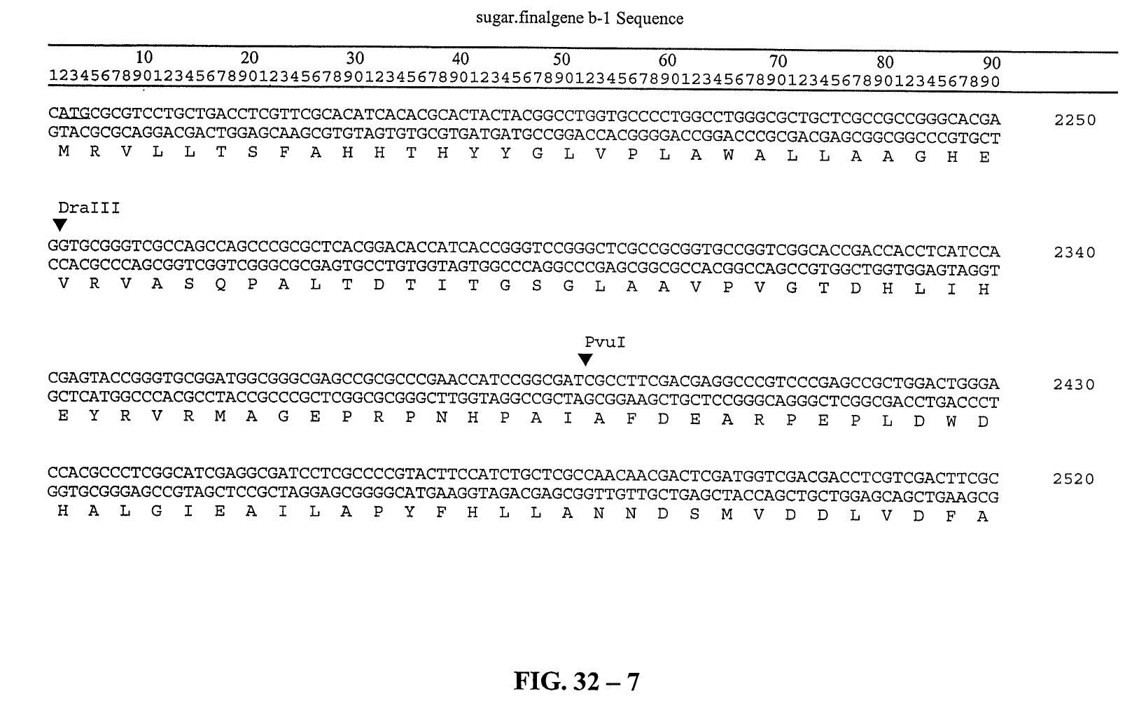

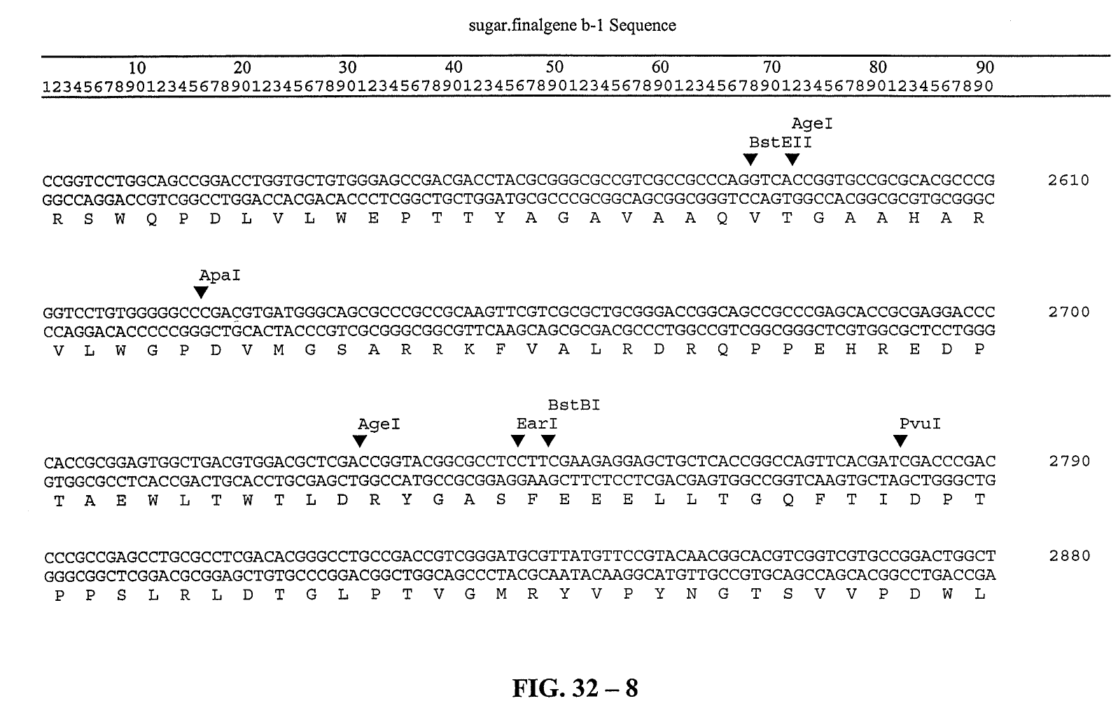

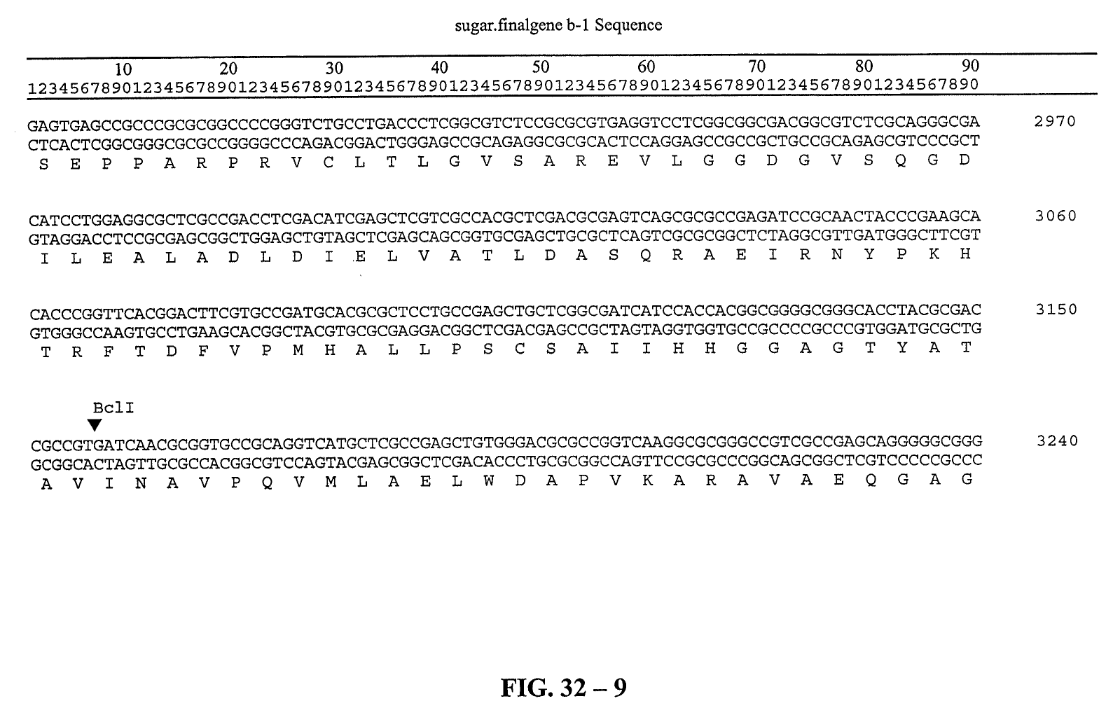

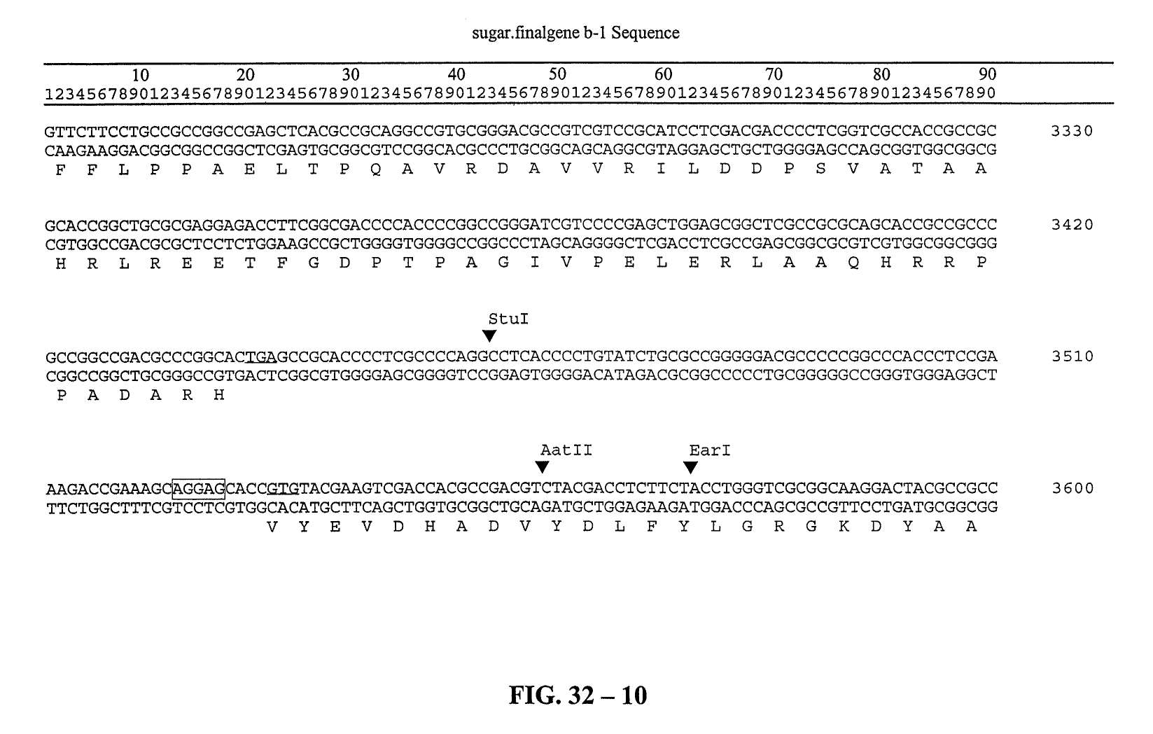

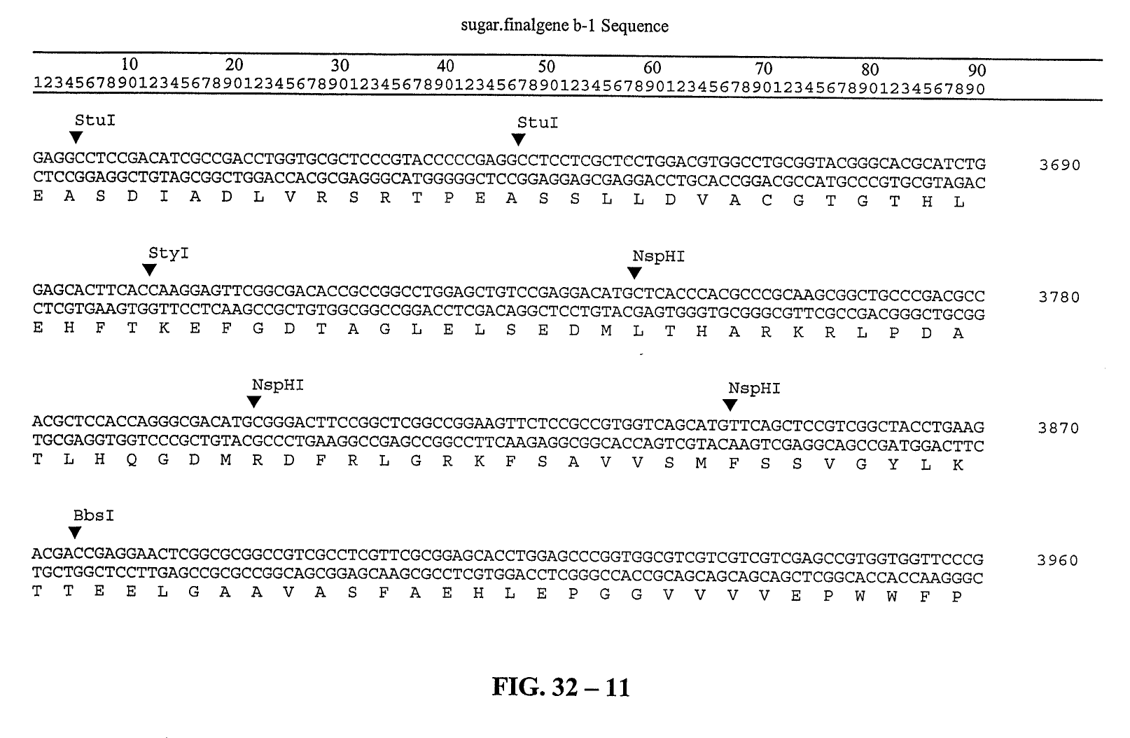

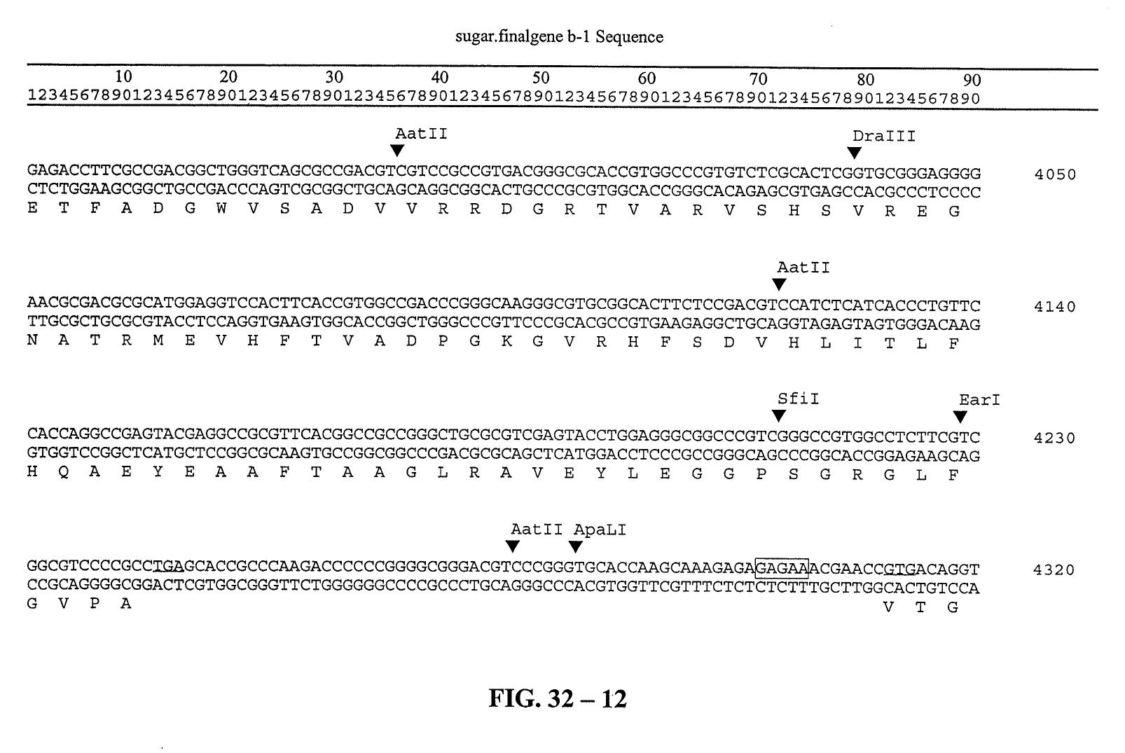

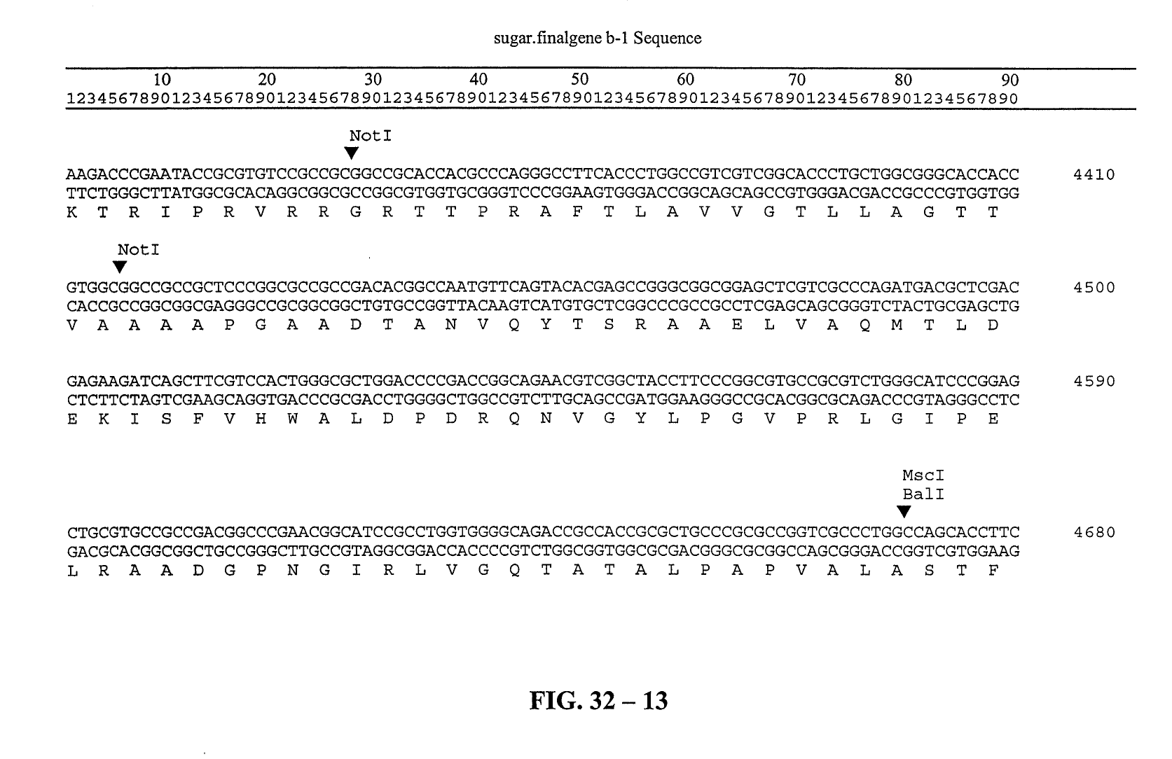

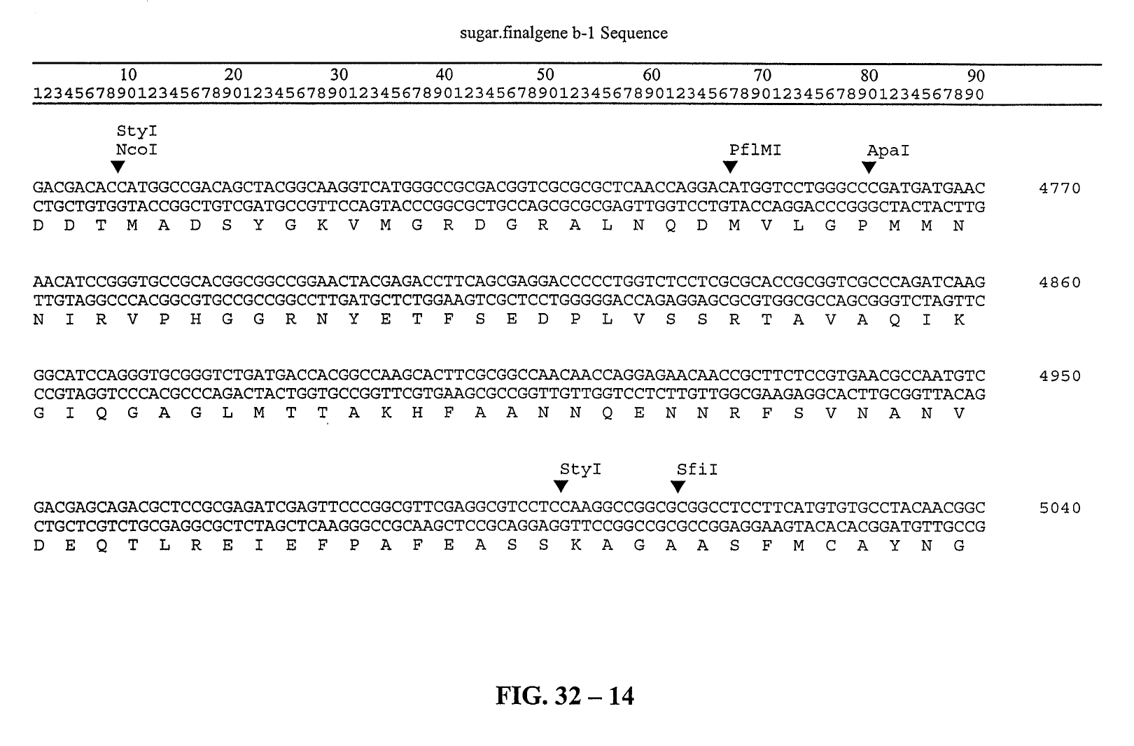

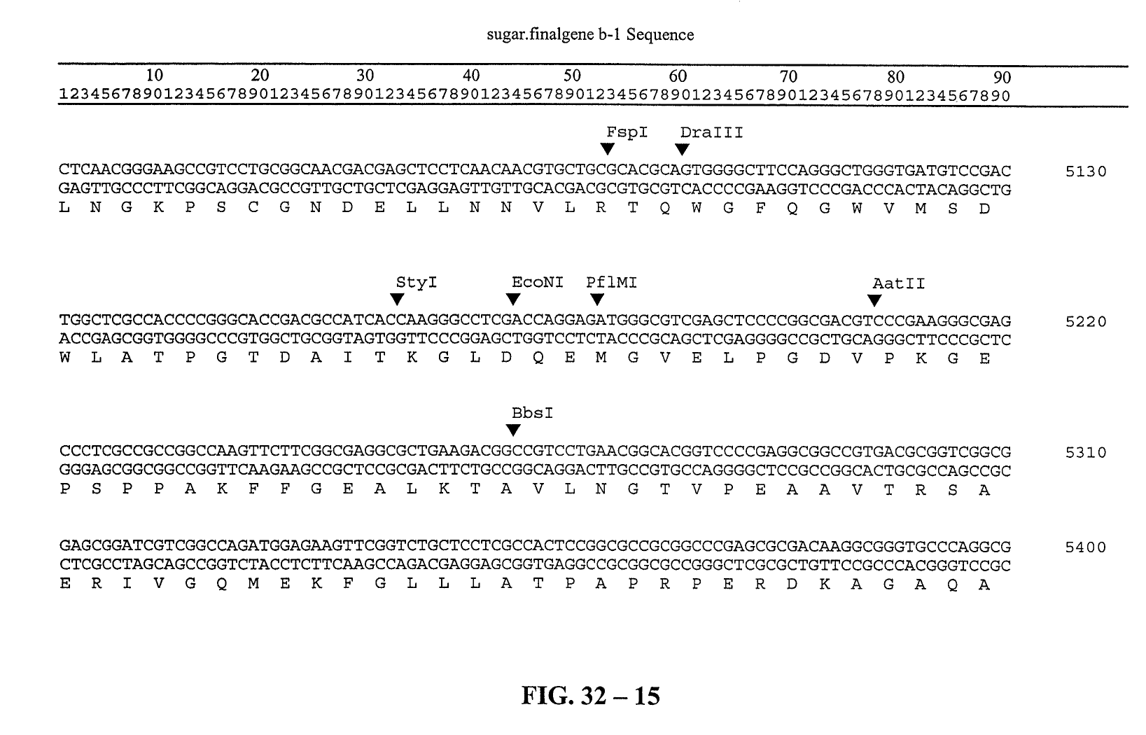

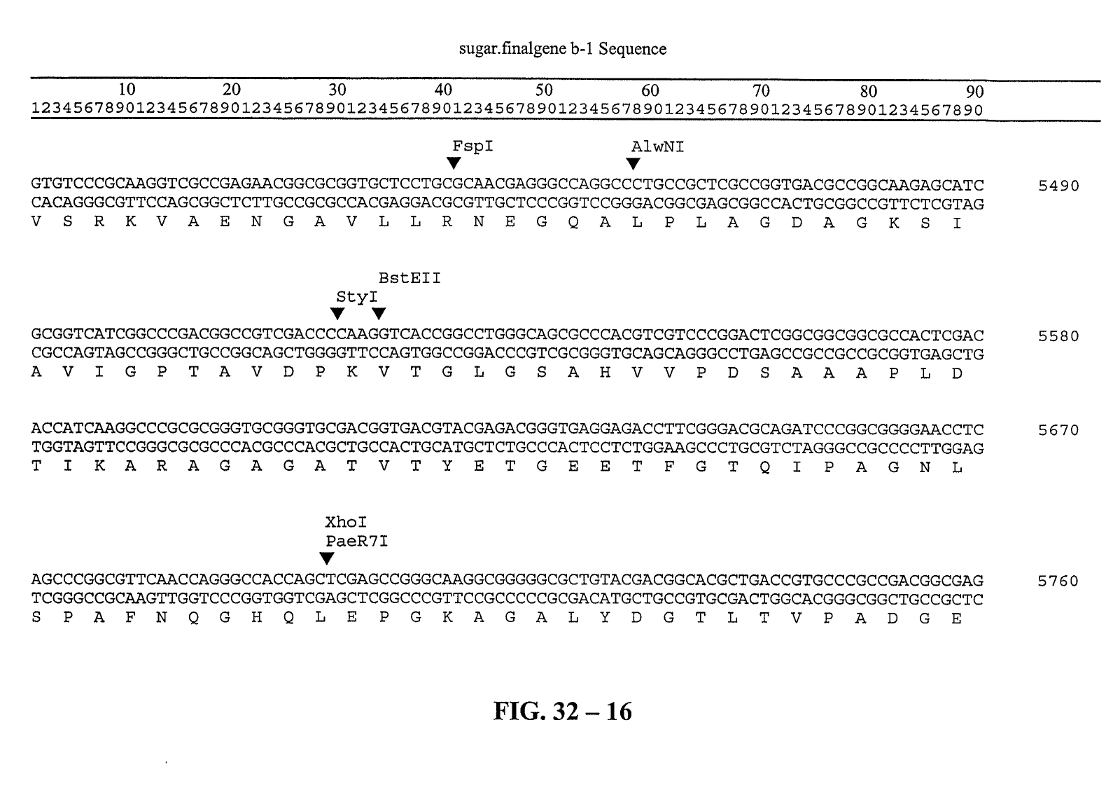

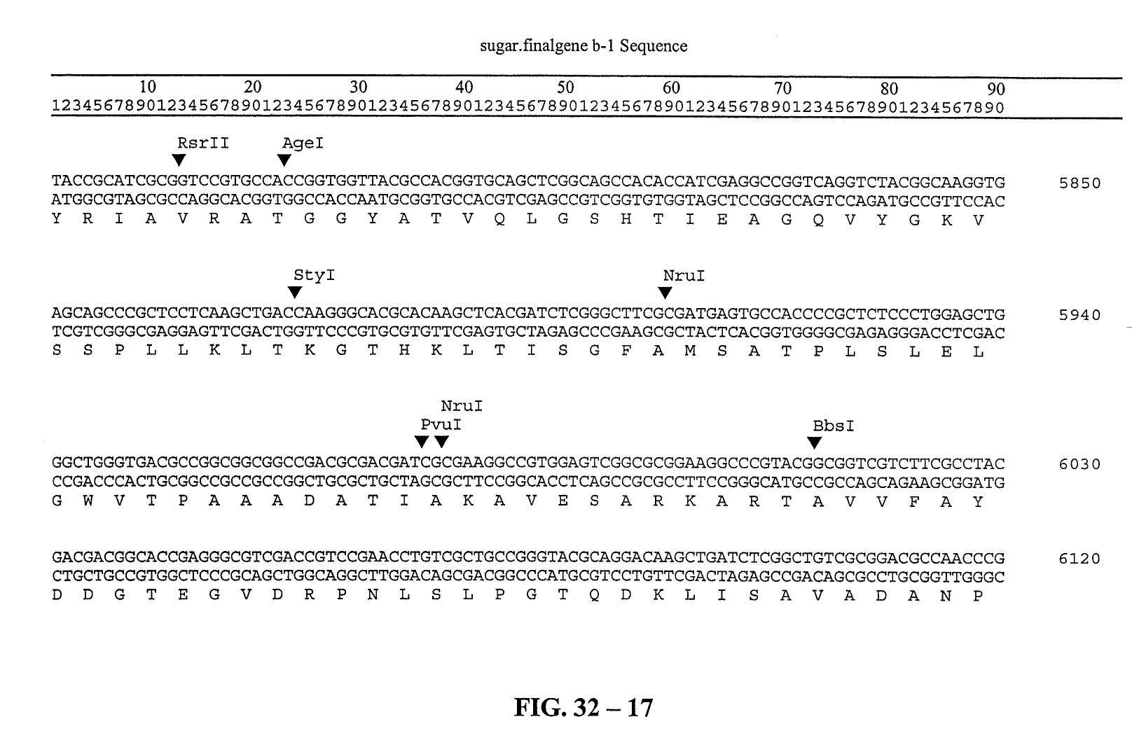

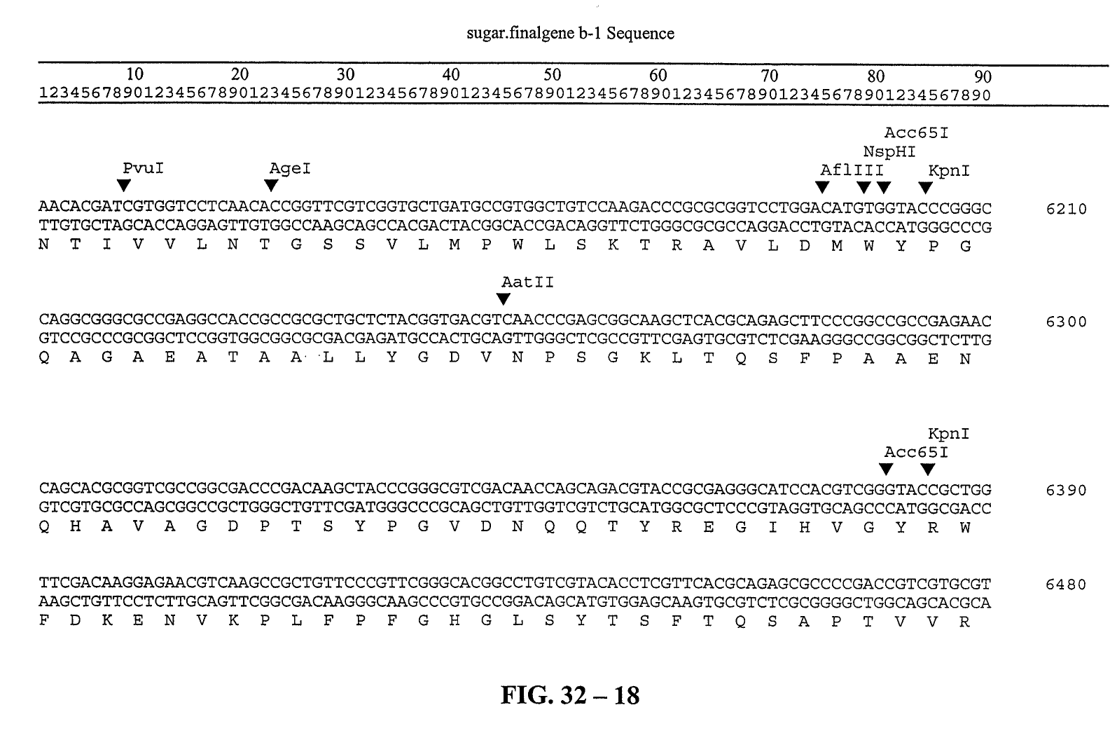

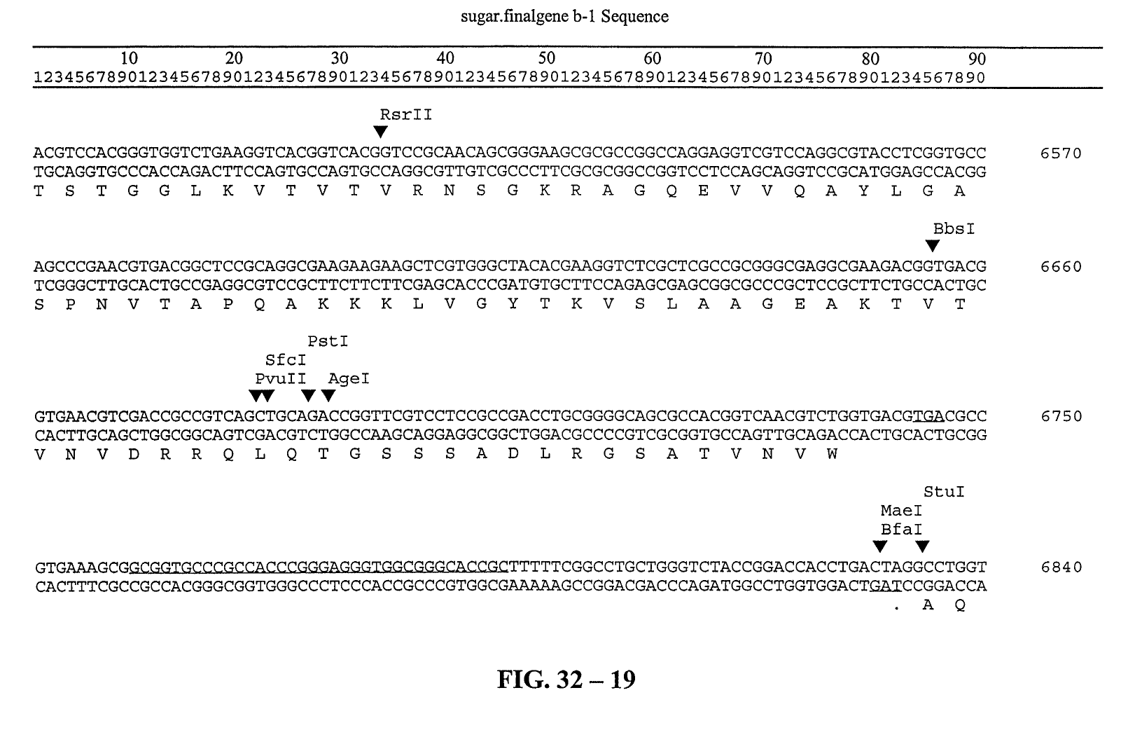

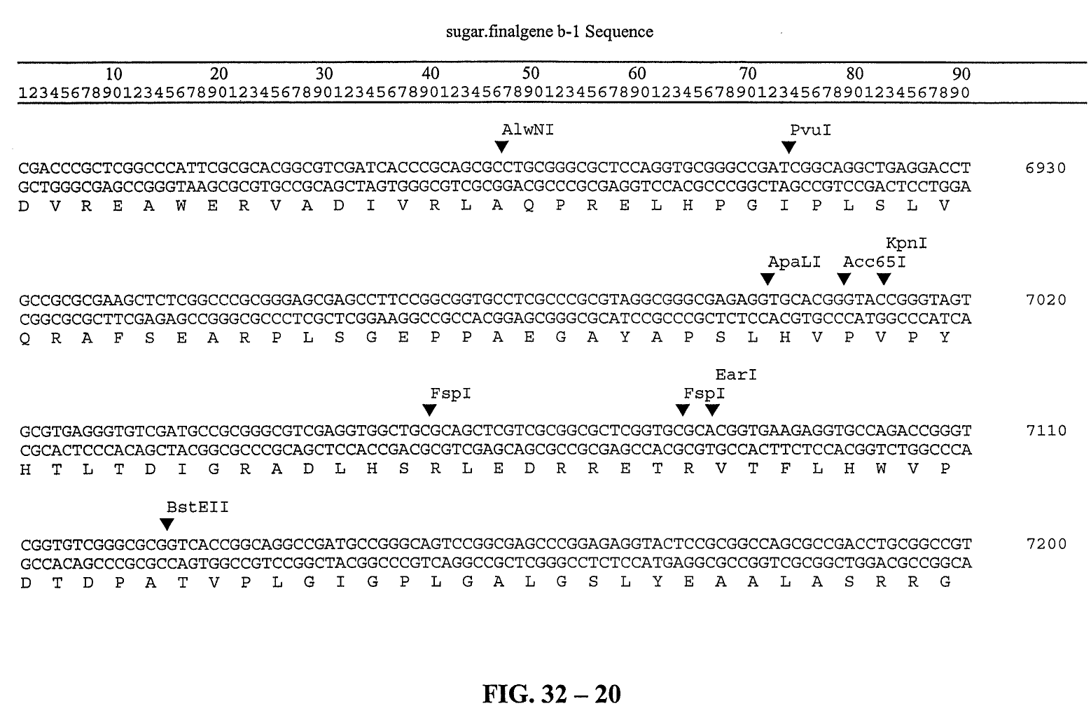

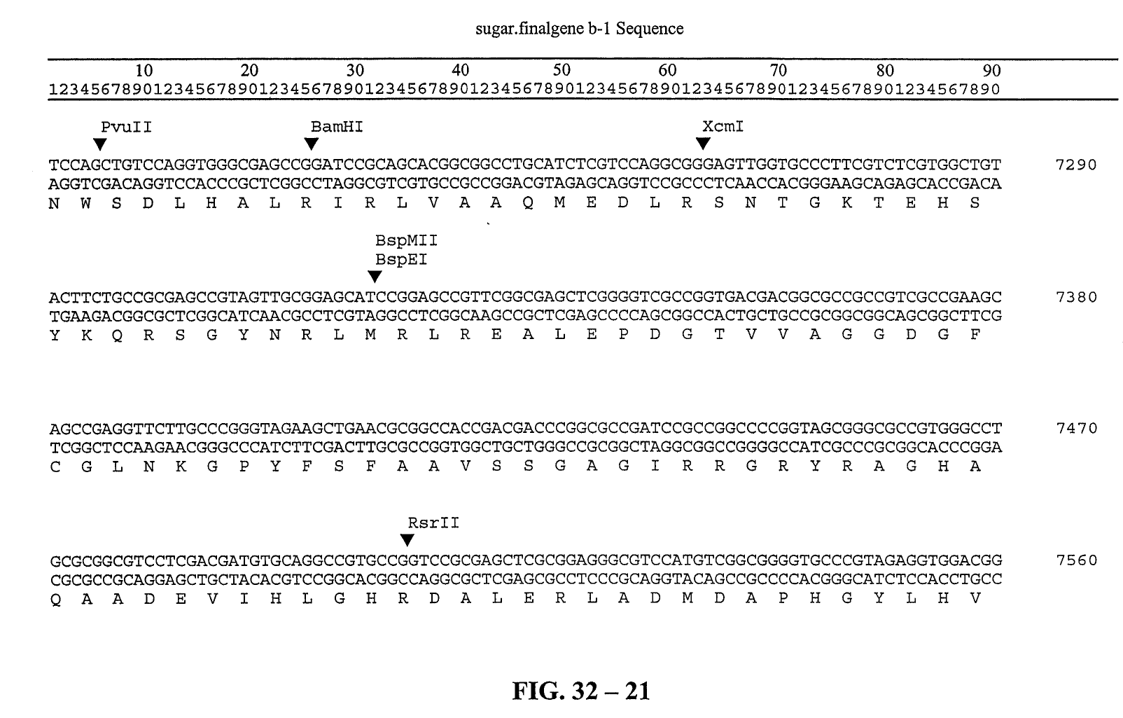

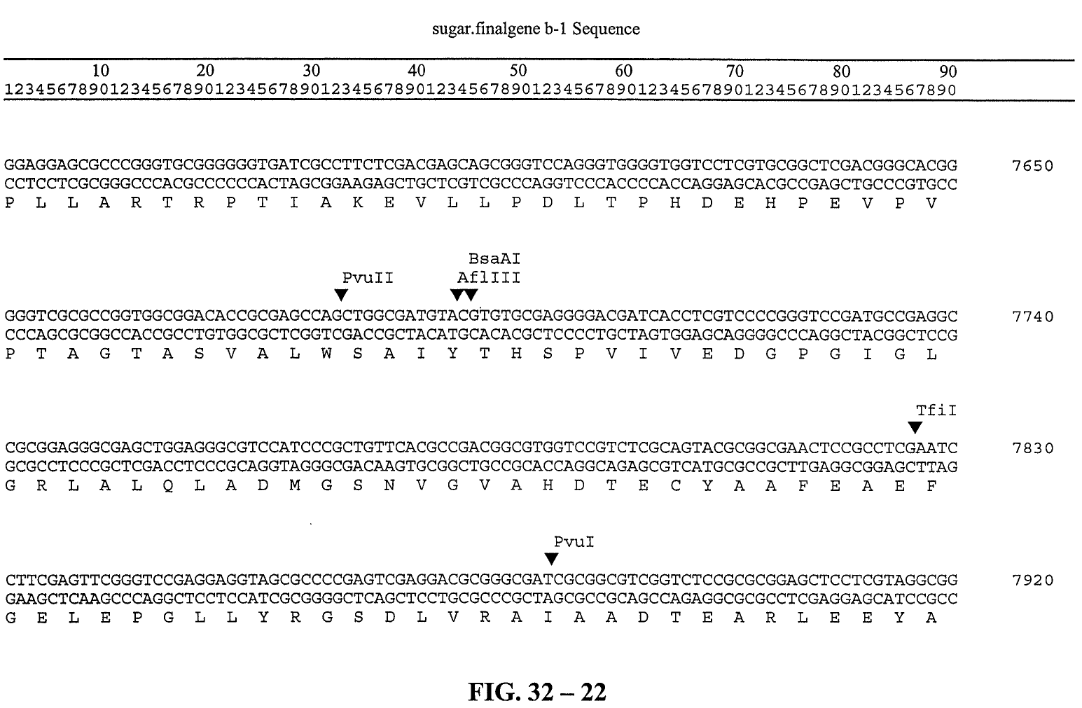

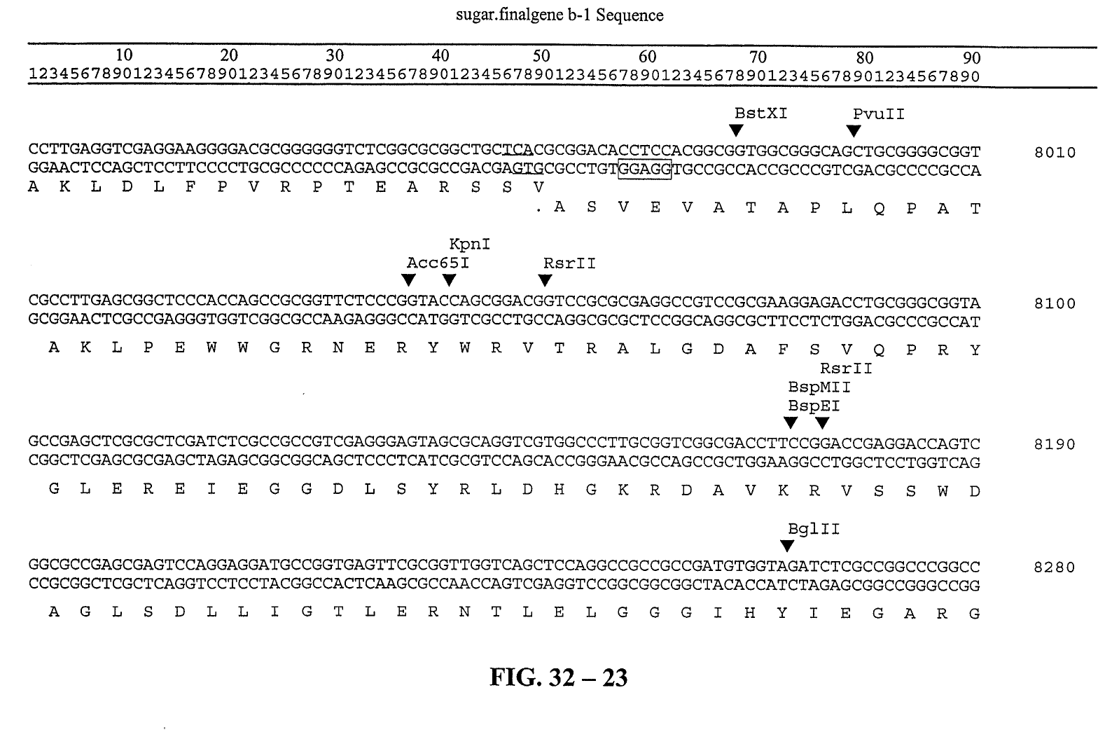

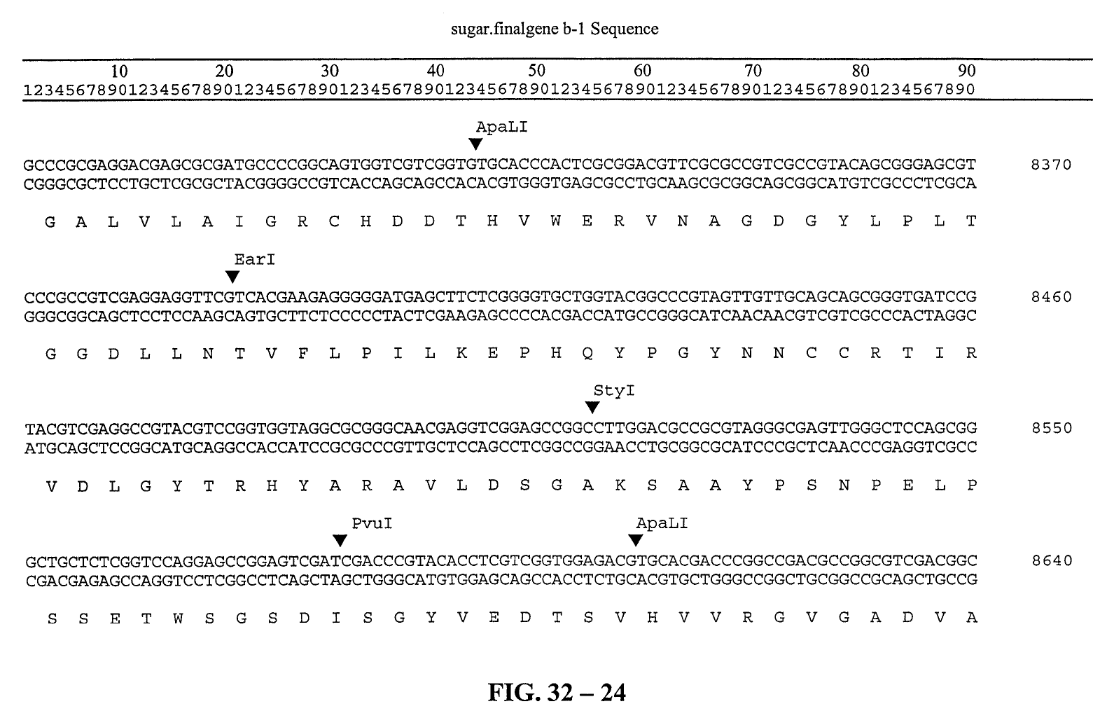

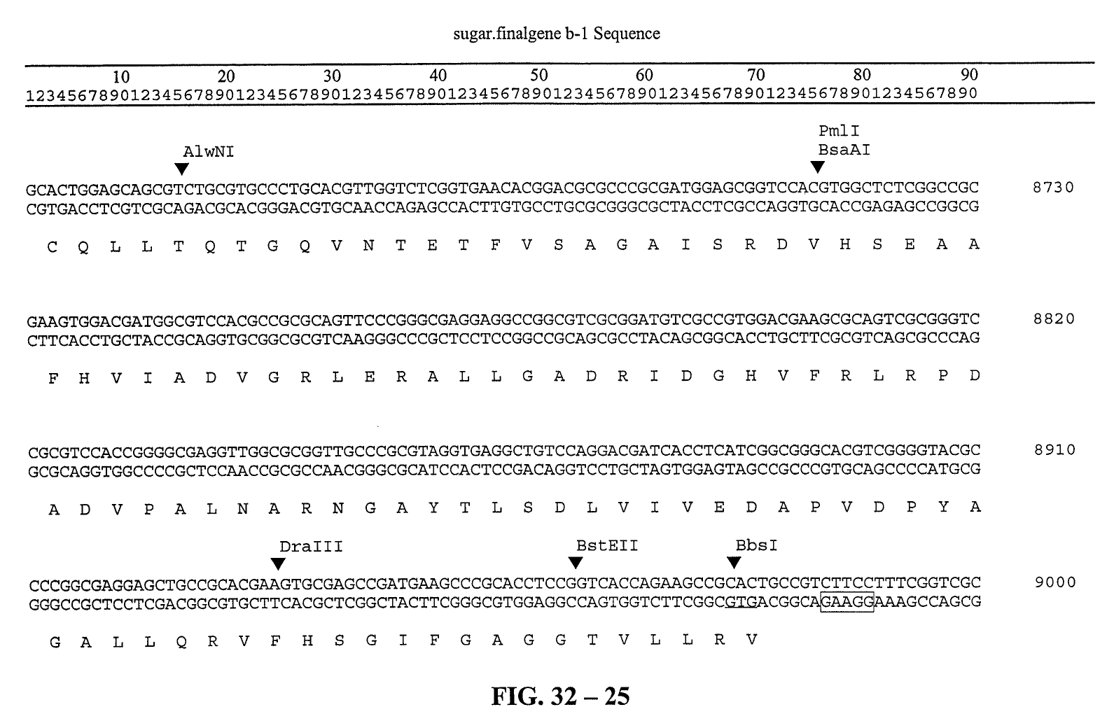

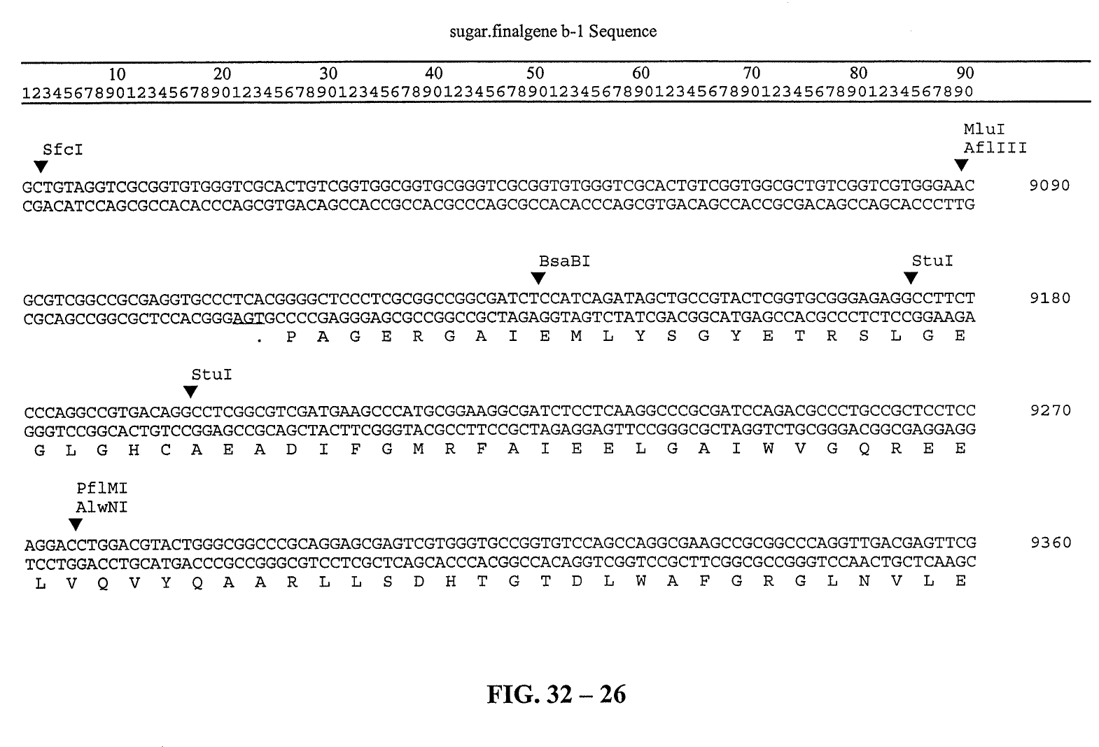

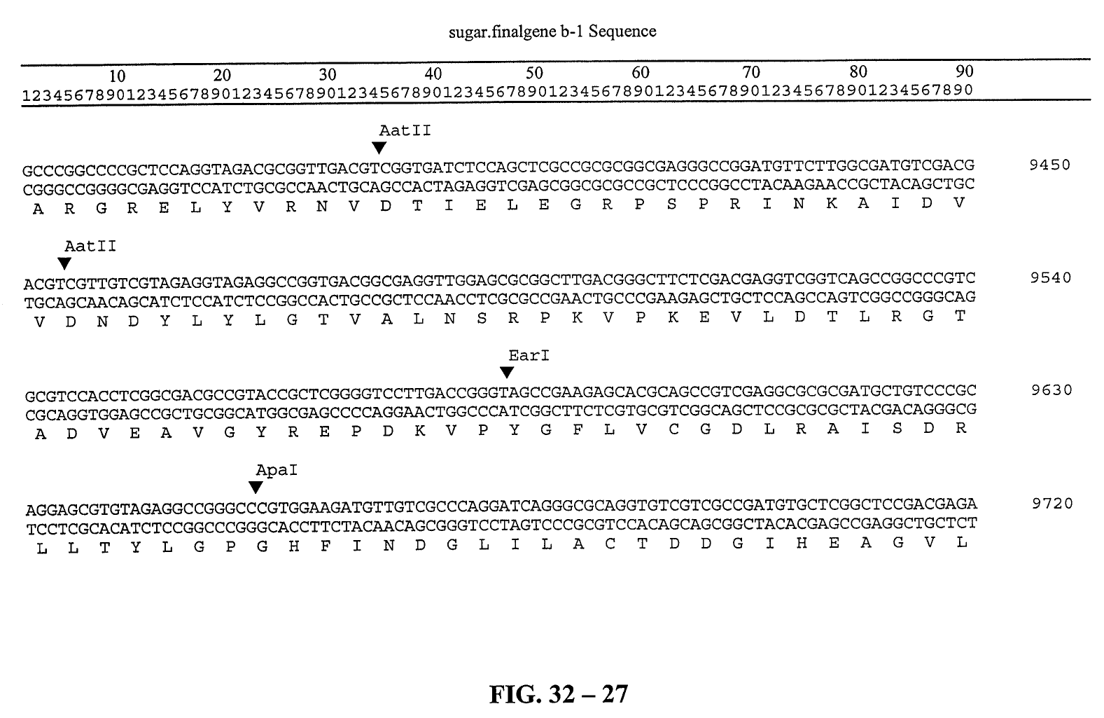

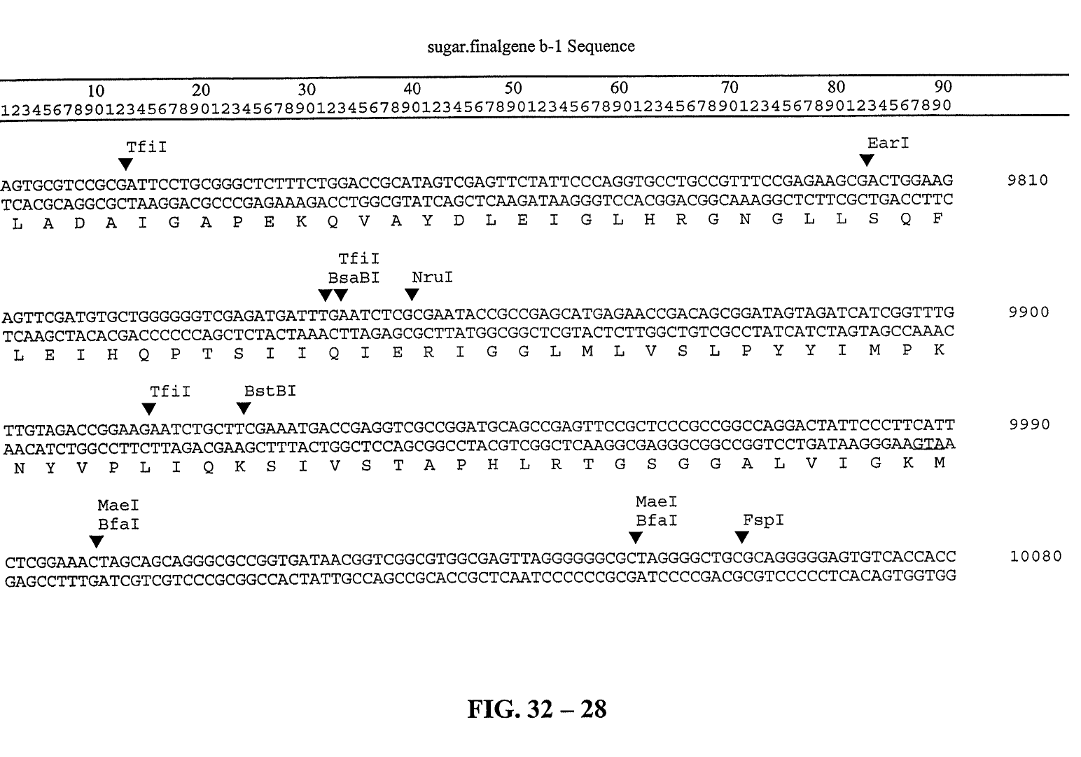

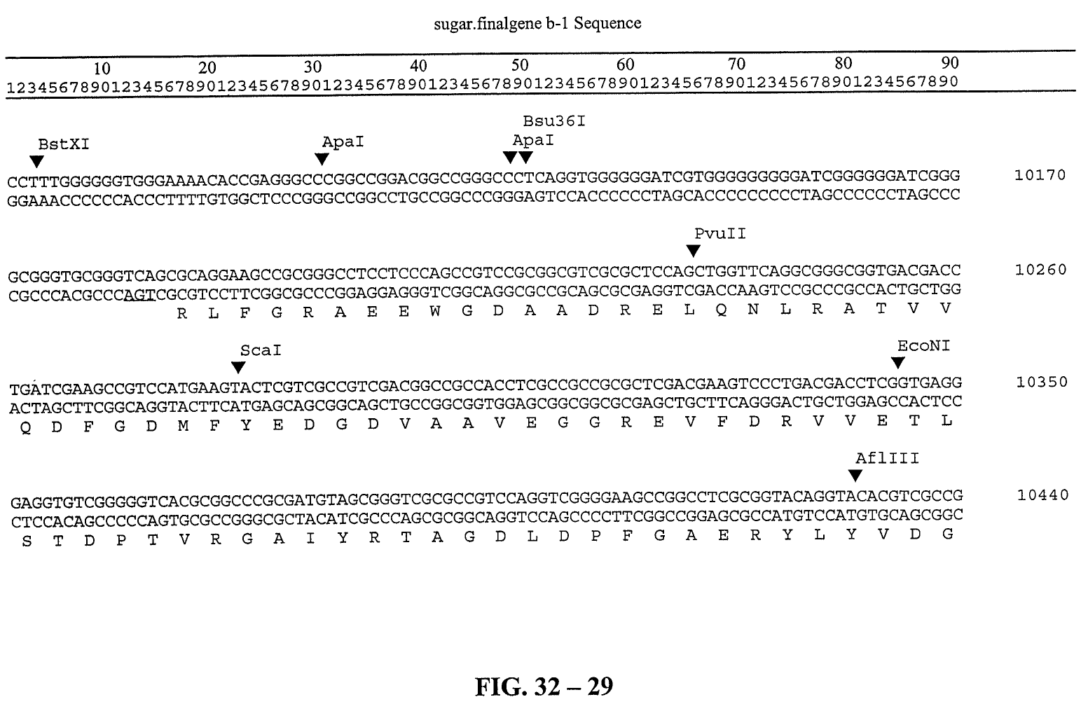

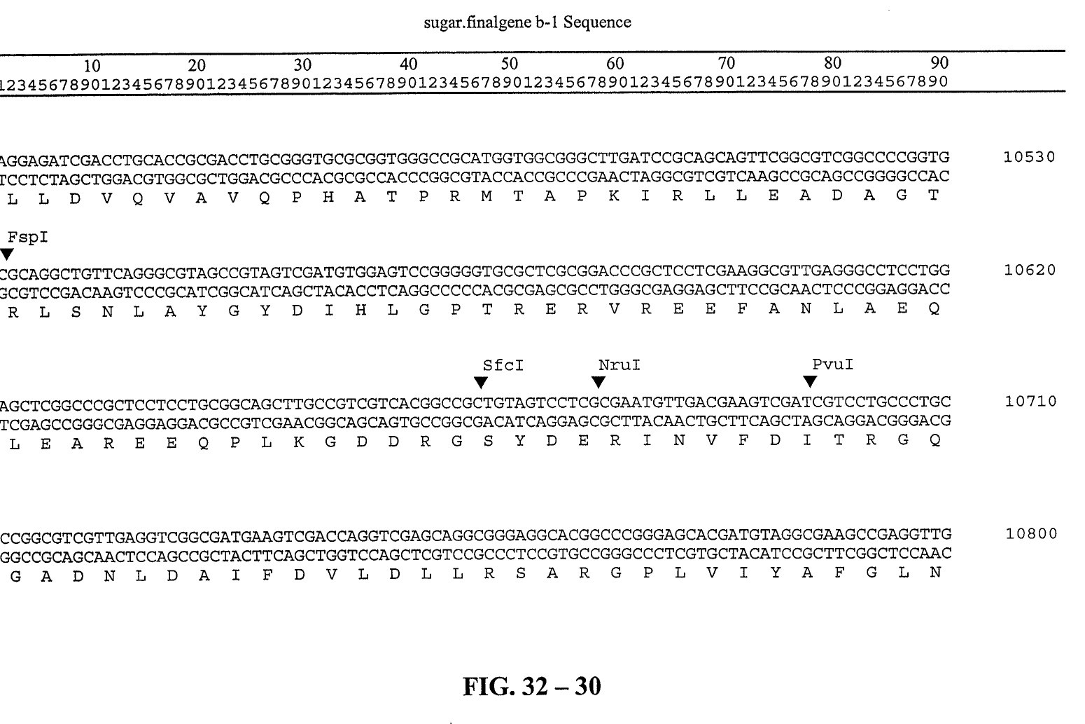

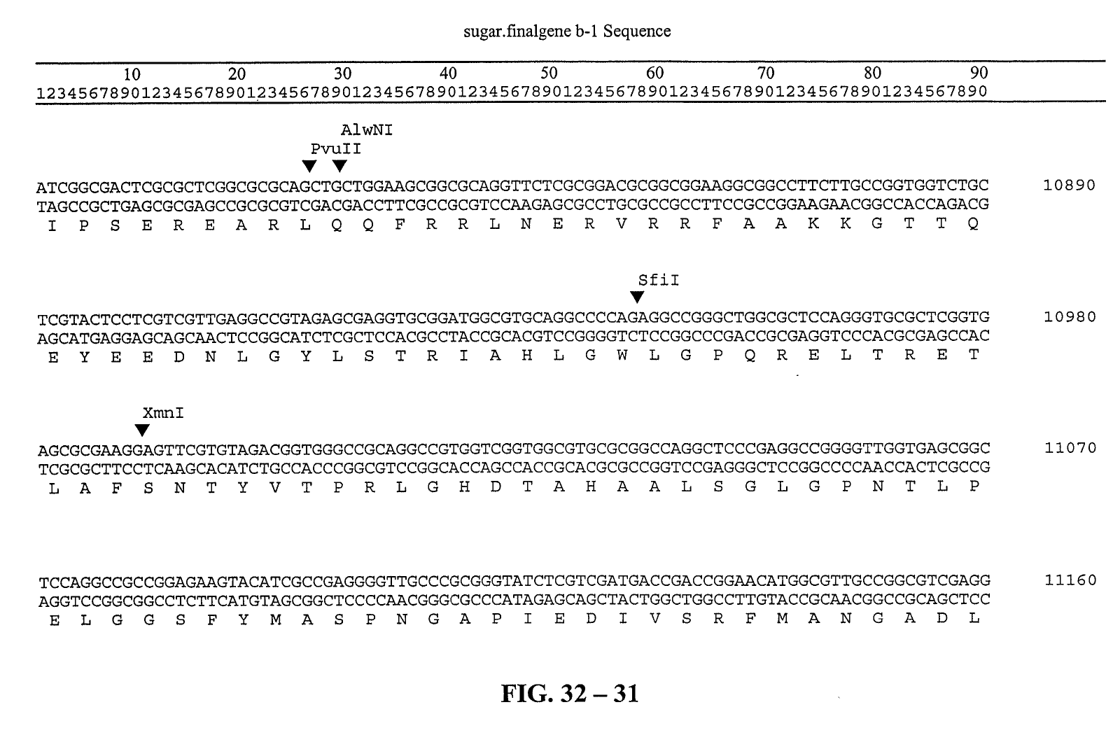

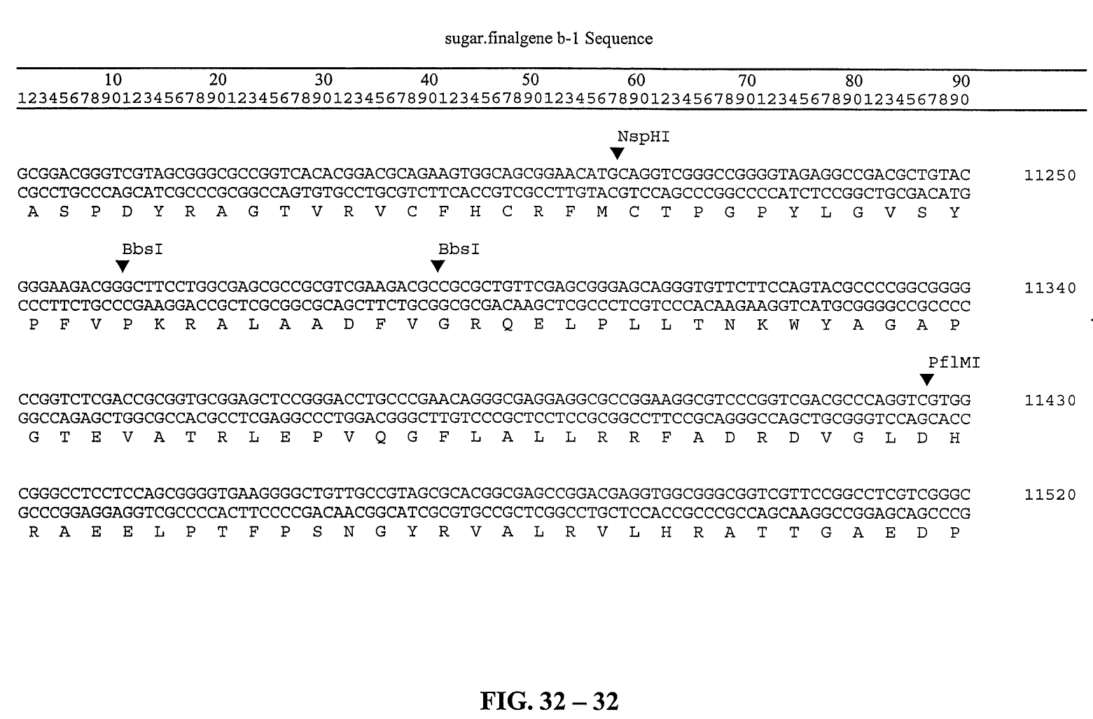

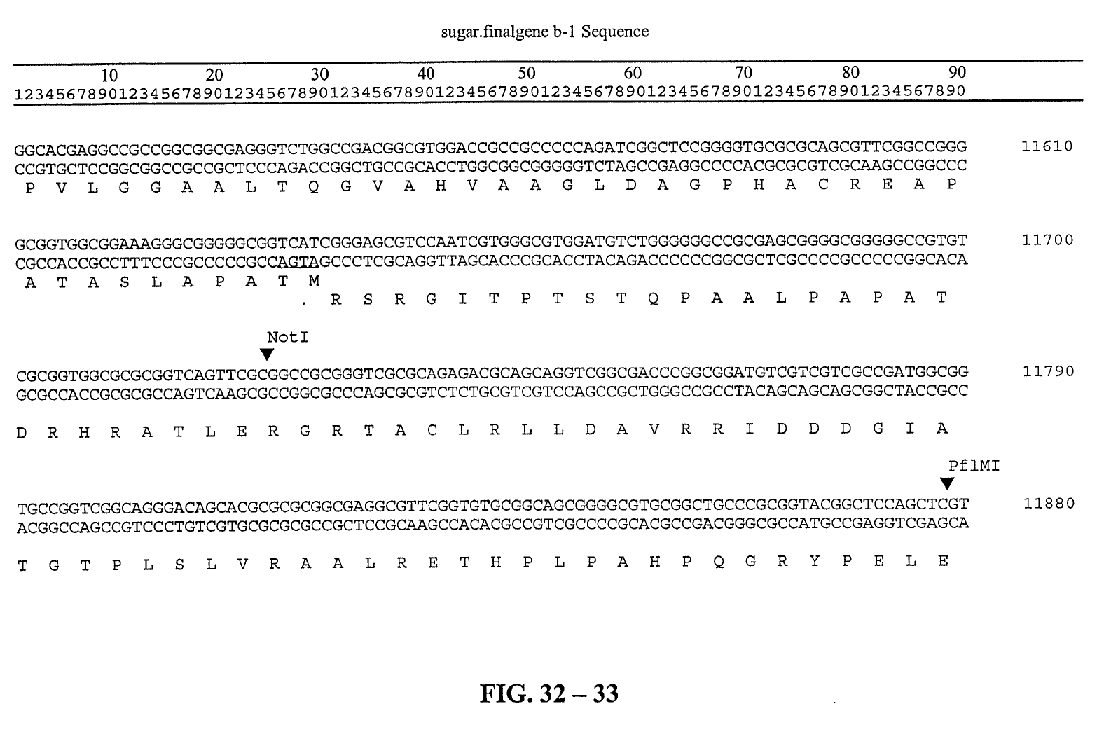

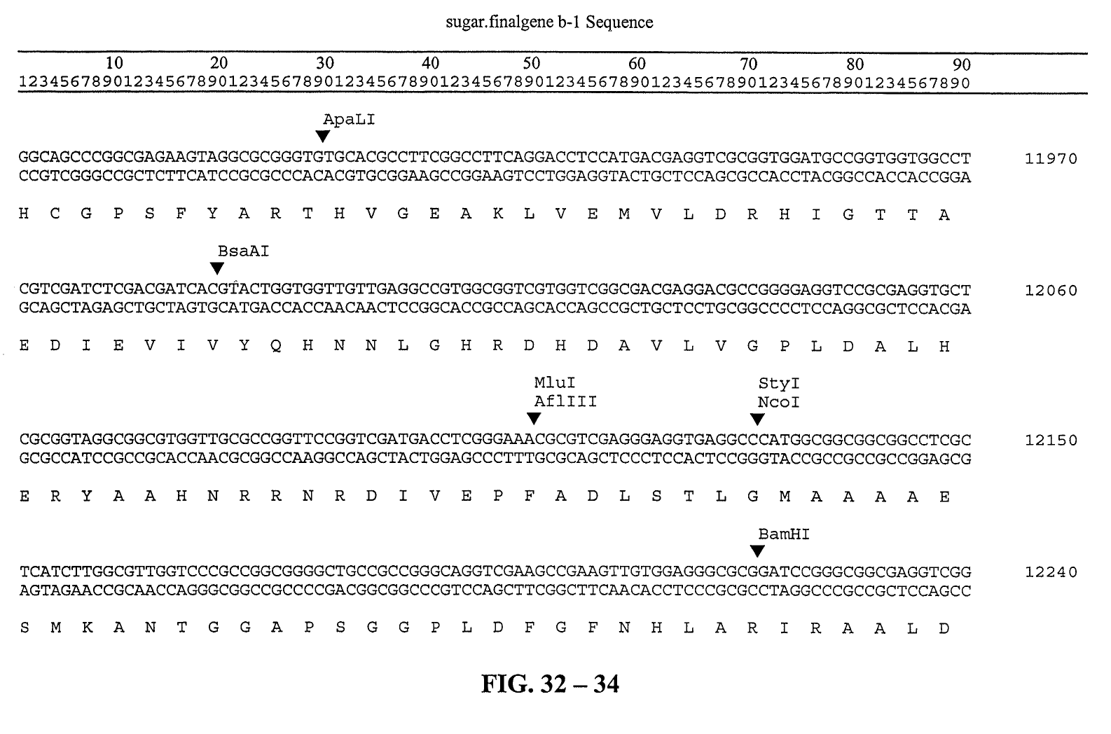

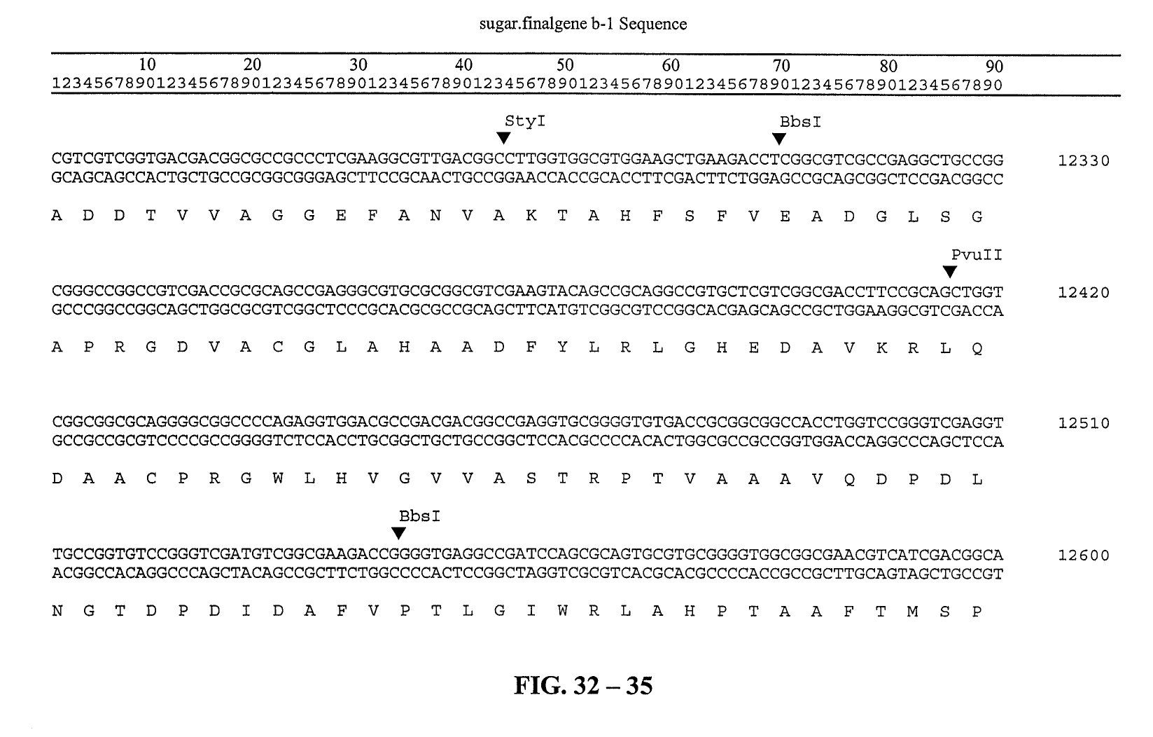

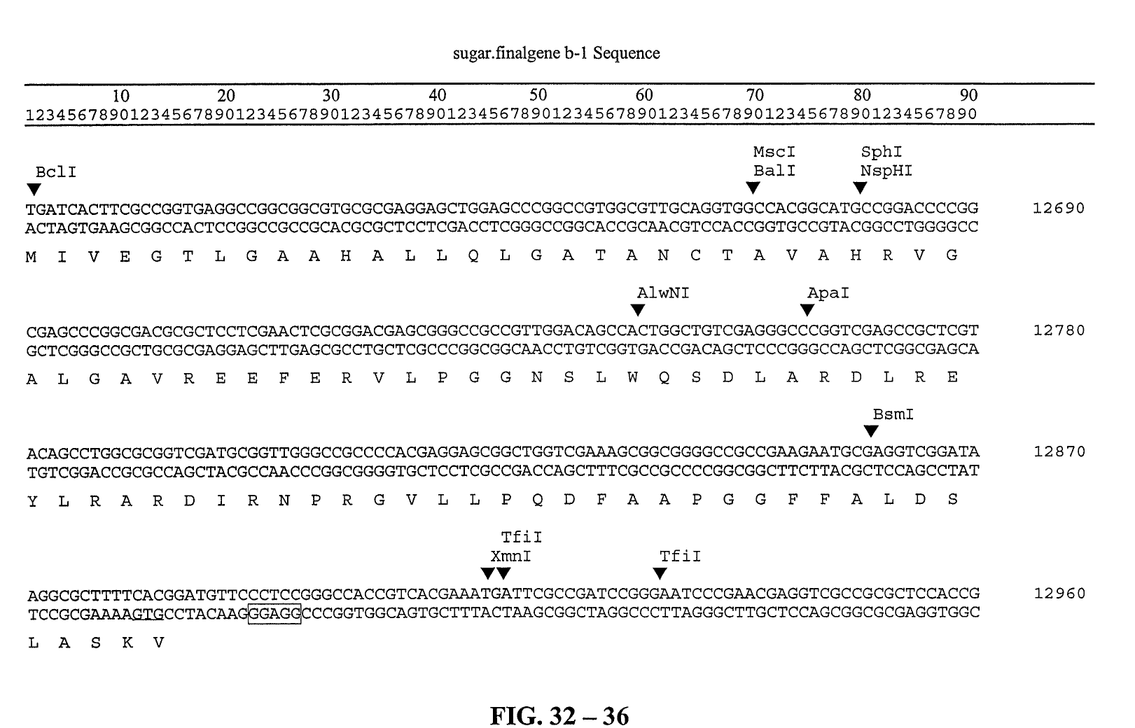

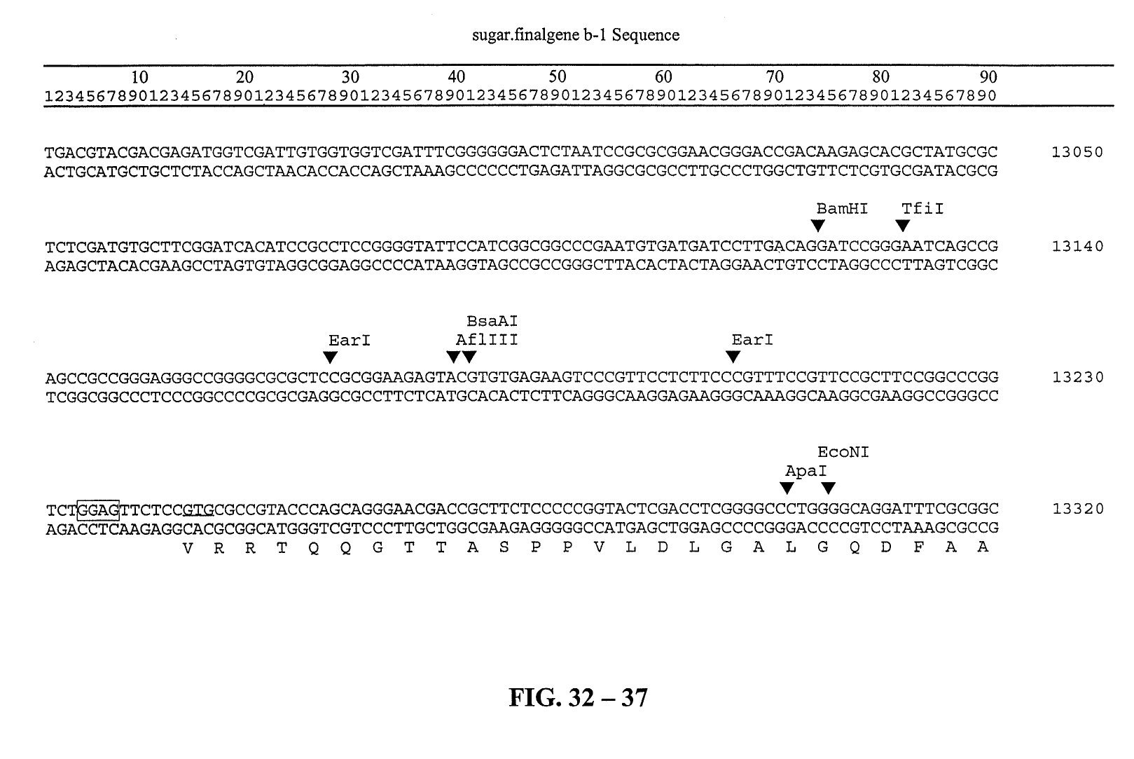

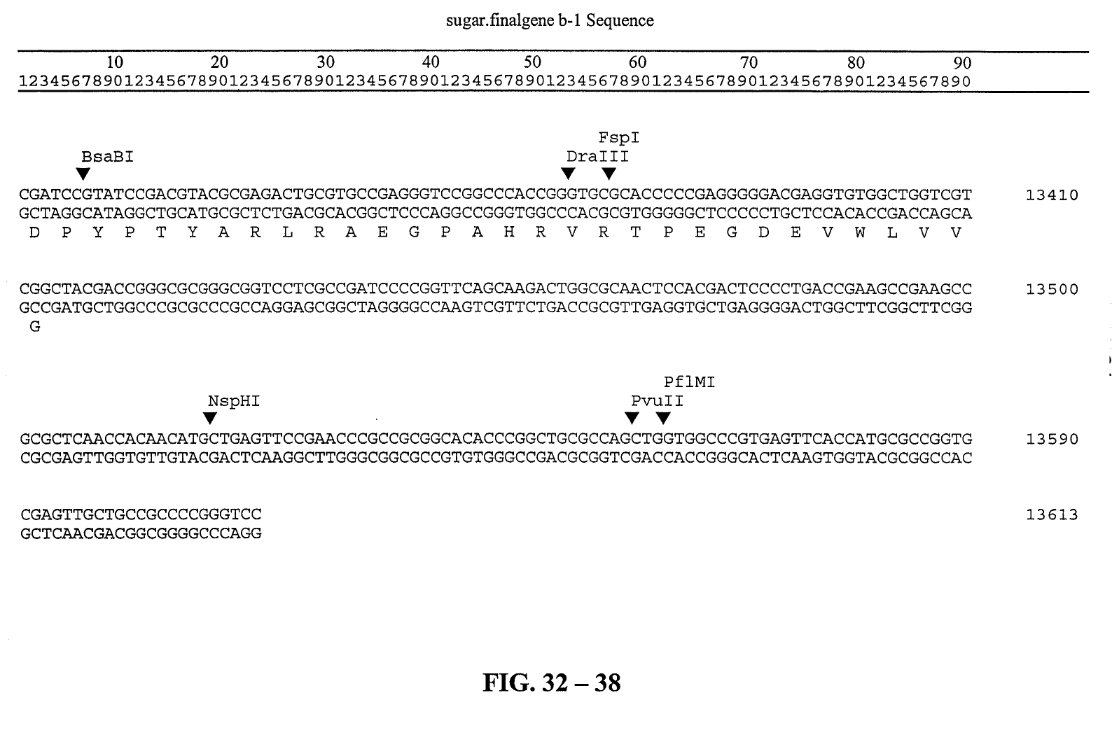

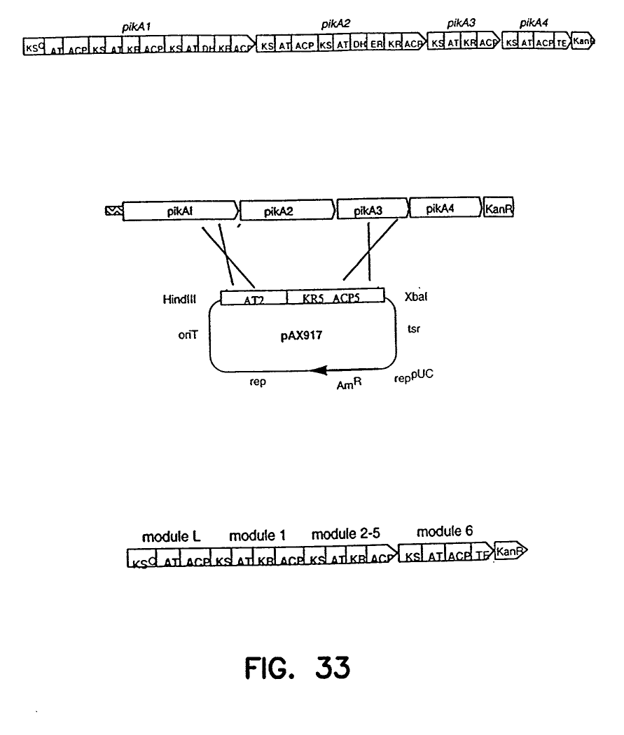

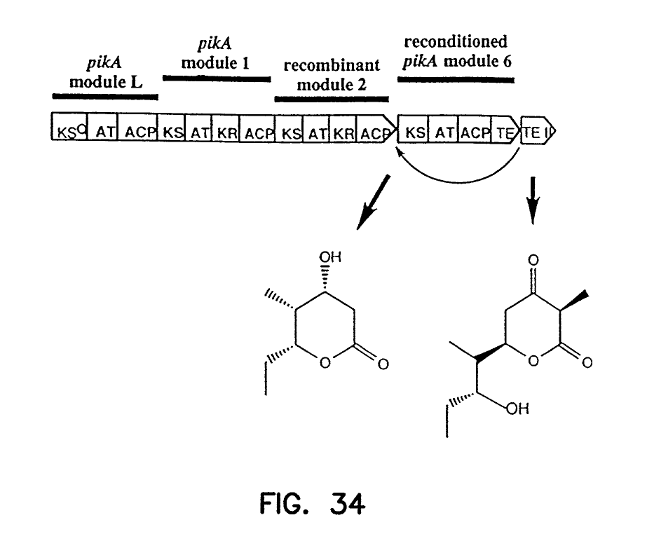

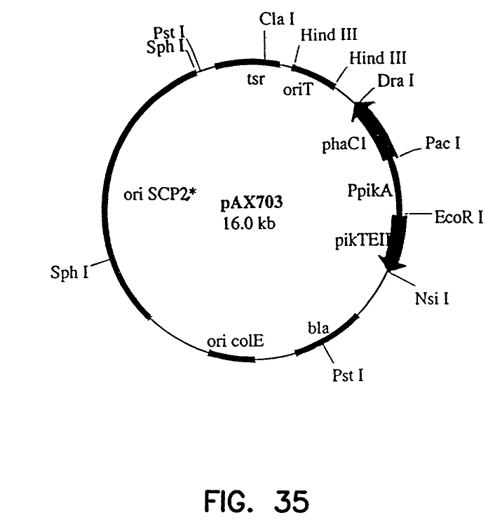

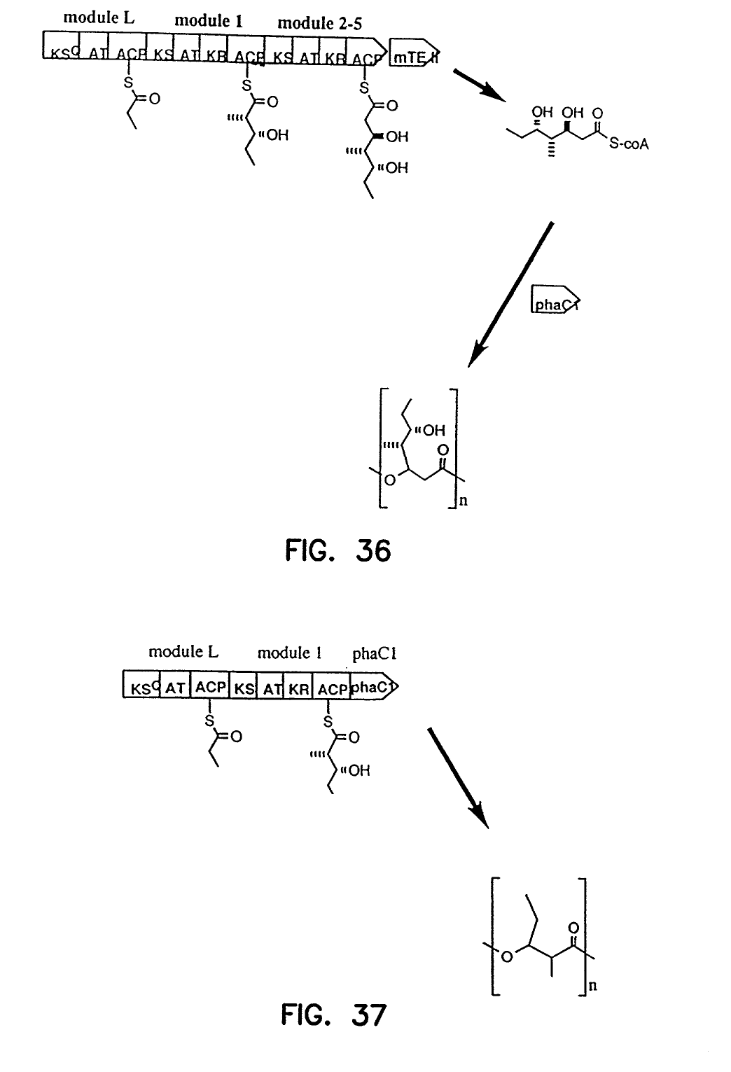

Initial analysis unveiled two significant architectural differences in the pikA-encoded PKS. First, compared with eryA (Donadio et al., 1998) and oleA (Swan et al., 1994), two PKS clusters that produce 14-membered ring macrolides erythromycin and oleadomycin similar to pikromycin, the presence of separate ORFs, pikAII and pikAIV, encoding Pik module 5 and Pik module 6 (as individual modules) as opposed to one bimodular protein as in eryAIII and oleAIII is striking. Secondly, the presence of a type II thioesterase immediately downstream of the type I PKS cluster is also unprecedented (FIG. 28). These two characteristics suggest that pikA may produce the 12-membered ring macrolactone 10-deoxymethynolide as well. Indeed, the domain organization of PikAI-AIII (module L-5) is consistent with the predicted biosynthesis of 10-deoxymethynolide except for the absence of a TE function at the C-terminus of Pik module 5 (PikAIII). The lack of a TE domain in PikAIII may be compensated by the type II TE (encoded by pikAV) immediately downstream of pikaAIV. Consistent with the supposition that two distinct polyketide ring systems are assembled from the pik PKS, two macrolide-lincosamide-streptogramin B type resistant genes, pikR1 and pikR2, are found upstream of the pik PKS (FIG. 29), which presumably provide cellular self-protection for S. venezuelae.

The genetic locus for desosamine biosynthesis and glycosyl transfer are immediately downstream of pikA. Seven genes, desI, desII, desIII, desIV, desV, desVI, and desVIII, are responsible for the biosynthesis of the deoxysugar, and the eighth gene, desVII, encodes a glycosyltransferase that apparently catalyzes transfer of desosamine onto the alternate (12- and 14-membered ring) polyketide aglycones. The existence of only one set of desosaimine genes indicates that DesVIII can accept both 10-deoxymethynolide and narbonolide as substrates (Jacobsen et al., 1997). The largest ORF in the des locus, desR, encodes a α-glycosidase that is involved in a drug inactivation-reactivation cycle for bacterial self-protection.

Just downstream of the des locus is a gene (pikC) encoding a cytochrome P450 hydroxylase similar to eryF (Andersen et al., 1992), and eryK (Stassi et al., 1993), PikC, and a gene (pikD) encoding a putative regulator protein, PikD (FIG. 28). Interestingly, PikC is the only P450 hydroxylase identified in the entire pik cluster, suggesting that the enzyme can accept both 12- and 14-membered ring macrolide substrates and, more remarkably, it is active on both C-10 and C-12 of the YC-17 (12-membered ring intermediate) to produce methymycin and neomethymycin (FIG. 30). PikD is a putative regulatory protein similar to ORFH in the rapamycin gene cluster (Schwecke et al., 1995).

The combined functionality coded by the eighteen genes in the pik cluster predicts biosynthesis of methymycin, neomethymycin, narbomycin and pikromycin (Table 2). Flanking the pik cluster locus are genes presumably involved in primary metabolism and genes that may be involved in both primary and secondary metabolism. An S-adenosyl-methionine synthase gene is located downstream of pikD that may help to provide the methyl group in desosamine synthesis. A threonine dehydratase gene was identified upstream of pikR1 that may provide precursors for polyketide biosynthesis. It is not apparent that any of these genes are dedicated to antibiotic biosynthesis and they are not directly linked to the pik cluster.

TABLE 2
Deduced function of ORFs in the pik cluster

| Polypeptide | Amino | Proposed function or | | | | | | |
|---|---|---|---|---|---|---|---|---|
| (ORF) | acids, no. | sequence similarity detected | | | | | | |
| PikAI | 4,613 | PKS | | | | | | |
| Loading | | KS^{Q} | AT(P) | | | | ACP | |
| module | | | | | | | | |
| Module 1 | | KS | AT(P) | | | KR | ACP | |
| Module 2 | | KS | AT(A) | DH | | KR | ACP | |
| PikAII | 3,739 | PKS | | | | | | |
| Module 3 | | KS | AT(P) | | | KR⁰ | ACP | |
| Module 4 | | KS | AT(P) | DH | ER | KR | ACP | |
| PikAIII | 1,562 | PKS | | | | | | |
| Module 5 | | KS | AT(P) | | | KR | ACP | |
| PikAIV | 1,346 | PKS | | | | | | |
| Module 6 | | KS | AT(P) | | | | ACP | TE |

| | | |
|---|---|---|
| PikAV | 281 | Thioesterase II (TEII) |
| DesI | 415 | 4-Dehydrase |
| DesII | 485 | Reductase? |
| DesIII | 292 | α-D-Glucose-1-phosphate |
| | | thymidylyltransferase |
| DesIV | 337 | TDP-glucose 4,6-dehydratase |
| DesV | 379 | Transaminase |
| DesVI | 237 | N,N-dimethyltransferase |
| DesVII | 426 | Glycosyl transferase |
| DesVIII | 402 | Tautomerase? |
| DesR | 809 | β-Glucosidase (involved in resistance |
| | | mechanism) |
| PikC | 418 | P450 hydroxylase |
| PikD | 945? | Putative regulator |
| PikR1 | 336 | rRNA methyltransferase (mls resistance) |
| PikR2 | 288? | rRNA methyltransferase (mls resistance) |

AT(A), acyltransferase incorporating an acetate extender unit;
AT(P), acyltransferase incorporating a propionate extender unit.
KR⁰, an inactive KR.
Enzymes of uncertain function are denoted with a question mark.

TABLE 3
Summary of mutational analyses of the pik cluster

| | | | Antibiotic production/ | |
|---|---|---|---|---|
| | Type of | Target | Intermediate accumulation | |
| Mutant | mutation | gene | Met & neomethymycin | Pikromycin |
| AX903 | Insertion | pikAI | No/No | No/No |
| LZ3001 | Deletion/ | desVI | No/10-deoxy- | No/narbonolide |
| | replacement | | methynolide | |
| LZ4001 | Deletion/ | desV | No/10-deoxy- | No/narbonolide |
| | replacement | | methynolide | |
| AX905 | Deletion/ | pikAV | <5%/No | <5%/No |
| | replacement | | | |
| AX906 | Insertion | pikC | No/YC-17 | No/narbomycin |

Mutational Analysis of the pik Cluster. Extensive disruption of genes in the pik cluster were carried out to address the role of key enzymes in antibiotic production (Table 3). First, PikAI, the first putative enzyme involved in the biosynthesis of 10-deoxymethynolide and narbonolide was inactivated by insertional mutagenesis. The resulting mutant, AX903, produced neither methymycin or neomethymycin, nor narbomycin or pikromycin, indicating that pikA encodes a PKS required for both 12- and 14-membered ring macrolactone formation.

Second, deletion of both desVI and desV abolished methymycin, neomethymycin, narbomycin and pikromycin production, and the resulting mutants, LZ3001 and LZ4001, accumulate 10-deoxymethynolide and narbonolide in their culture broth, indicating that enzymes for desosamine synthesis and transfer are also shared by the 12- and 14-membered ring macrolides.

In order to understand the mechanism of polyketide chain termination at PikAII (PIKAIII (module 5) is presumed to be the termination point in construction of 10-deoxymethynolide), the pik TEII gene, pikAV, was deleted. The deletion/replacement mutant, AX905, produces less than 5% of methymycin, neomethymycin, and less than 5% of pikromycin compared to wild type S. venezuelae. This abrogation in product formation occurs without significant accumulation of the expected aglycone intermediates, suggesting thatpik TEII is involved in the termination of 12- as well as 14-membered ring macrolides at PikAIII and PikAIV, respectively. Although the polar effects may influence the observed phenotype in AX905, this has been ruled out after the consideration of mutant LZ3001, in which mutation in an enzyme downstream of pikA V accumulated 10-deoxymethynolide and narbonolide. The fact that mutant AX905 failed to accumulate these intermediates suggested that the polyketide chains were not efficiently released from this PKS protein in the absence of Pik TEII. Therefore, Pik TEII plays a crucial role in polyketide chain release and cyclization, and it presumably provides the mechanism for alternative termination in pik polyketide biosynthesis.

Finally, disruption of pikC confirmed that PikC is the sole enzyme catalyzing hydroxylation of both YC-17 (at C-10 and C-12) and narbomycin (at C-12). The relaxed substrate specificity of PikC and its regional specificity at C-10 and C-12 provide another layer of metabolite diversity in the pik-encoded biosynthetic system.

Discussion

The work described herein has established that methymycin, neomethymycin, narbomycin and pikromycin biosynthesis is encoded by the pik cluster in S. venezuelae. Three key enzymes as well as the unique architecture of the cluster enable this relatively compact system to produce multiple macrolide antibiotics. Foremost, the presence of pik module 5 and 6 as separate proteins, PikAIII and PikAIV, and the activity of pik TEII enable the bacterium to terminate the polyketide chain at two different points of assembly, thereby producing two macrolactones of different ring size. Second, DesVII, the glycosyltransferase in the pik cluster, can accept both 12- and 14-membered ring macrolactones as substrates. Finally, PikC, the P450 hydroxylase, has a remarkable substrate and regiochemical specificity that introduces another layer of diversity into the system.

It is interesting to consider that pikA evolved in a line analogous to eryA and oleA since each of these PKSs specify the synthesis of 14-membered ring macrolactones. Therefore, pik may have acquired the capacity to generate methymycin when a mutation in the primordial pikAIII-pikAIV linker region caused splitting of Pik module 5 and 6 into two separate gene products. This notion is raised by two features of the nucleotide sequence. First, the intergenic region between pikAIII and pikAIV, which is 105 bp, may be the remanent of an intramodular linker peptide of 35 amino acids. Moreover, the potential for independently regulated expression of pikAIV is implied by the presence of a 100 nucleotide region at the 5′ end of the gene that is relatively AT-rich (62% as comparing 74% G+C content in coding region). Thus, as the mutation in an original ORF encoding the bimodular multifunctional protein (PikAII-PikAIV) occurred, so too may have evolved a mechanism for regulated synthesis of the new gene product (PikAIV).

The role of Pik TEII in alternative termination of polyketide chain elongation intermediates provides a unique aspect of diversity generation in natural product biosynthesis. Engineered polyketides of different chain length are typically generated by moving the TE catalytic domain to alternate positions in a modular PKS (Cortes et al., 1995). Repositioning of the TE domain necessarily abolishes production of the original full-length polyketide so only one macrolide is produced each time. In contrast to the fixed-position TE domain, the independent Pik TEII polypeptide presumably has the flexibility to catalyze termination at different stages of polyketide assembly, therefore enabling the system to produce multiple products of variant chain length. Combinatorial biology technologies can now exploit this system for generating molecular diversity through construction of novel PKS systems with TEIIs for simultaneous production of several new molecules as opposed to the TE domains alone that limit catalysis to a single termination step.

It is noteworthy that sequences similar to Pik TEII are found in almost all known polyketide and non-ribosomal polypeptide biosynthetic systems (Marahiel et al., 1997). Currently, the pik TEII is the first to be characterized in a modular PKS. However, recent work on a TEII gene in the lipopeptide surfactin biosynthetic cluster (Schneider et al., 1998) demonstrated that srf-TEII plays an important role in polypeptide chain release, and may suggest that srf-TEII reacts at multiple stages in peptide assembly as well (Marahiel et al., 1997).

The enzymes involved in post-polyketide assembly of 10-deoxymethynolide and narbonolide are particularly intriguing, especially the glycosyltransferase, DesVII, and P450 hydroxylase, PikC. Both have the remarkable ability to accept substrates with significant structural variability. Moreover, disruption of desVI demonstrated that DesVII also tolerates variations in deoxysugar structure (Example 6). Likewise, PikC has recently been shown to convert YC-17 to methymycin/neomethymycin and narbomycin to pikromycin in vitro.

Targeted gene disruption of ORF1 abolished both pikromycin and methymycin production, indicating that the single cluster is responsible for biosynthesis of both antibiotics. Deletion of the TE2 gene substantially reduced methymycin and pikromycin production, which demonstrates that TE2, in contrast to the position-fixed TE1 domain, has the capacity to release polyketide chain at different points during the assembly process, thereby producing polyketides of different chain length.

The results described above were unexpected in that it was surprising that one PKS cluster produces two macrolides which differ in the number of atoms in their ring structure, that module 5 and module 6 of the PKS are in ORFs that are separated by a spacer region, that PikAIII lacked TE, that there was a Type II thioesterase, that TEI domain was not separate, and that 2 resistance genes were identified which may be specific for either a 12- or 14-membered ring.

With eighteen genes spanning less than 60 kb of DNA capable of producing four active macrolide antibiotics, the pik cluster represents the least complex yet most versatile modular PKS system so far investigated. This simplicity provides the basis for a compelling expression system in which novel active ketoside products are engineered and produced with considerable facility for discovery of a diverse range of new biologically active compounds.

### Summary

Complex polyketide synthesis follows a processive reaction mechanism, and each module within a PKS harbors a string of three to six enzymatic domains that catalyze reactions in nearly linear order as described in particular detail for the erythromycin-producing PKS (Katz, 1997; Khosla, 1997; Staunton et al. 1997). The combined set of PKS modules and catalytic domains along with genes that encode enzymes for post-polyketide tailoring (e.g., glycosyl transferases, hydroxylases) typically limits a biosynthetic system to the generation of a single polyketide product.

Combinatorial biology involves the genetic manipulation of multistep biosynthetic pathways to create molecular diversity in natural products for use in novel drug discovery. PKSs represent one of the most amenable systems for combinatorial technologies because of their inherent genetic organization and ability to produce polyketide metabolites, a large group of natural products generated by bacteria (primarily actinomycetes and myxobacteria) and fungi with diverse structures and biological activities. Complex polyketides are produced by multifunctional PKSs involving a mechanism similar to long-chain fatty acid synthesis in animals (Hopwood et al., 1990). Pioneering studies (Cortes et al., 1990; Donadio et al., 1991) on the erythromycin PKS in Saccharopolyspora erythraea revealed a modular organization. Characterization of this multidomain protein system, followed by molecular analysis of rapamycin (Aparicio et al., 1996), FK506 (Motamedi et al., 1997), soraphen A (Schupp et al., 1995), niddamycin (Kakavas et al., 1997), and rifarnycin (August et al., 1998) PKSs, demonstrated a co-linear relationship between modular structure of a multifunctional bacterial PKS and the structure of its polyketide product.

In a survey of microbial systems capable of generating unusual metabolite structural variability, Streptomyces venezuelae ATCC 15439 is notable in its ability to produce two distinct groups of macrolide antibiotics. Methymycin and neomethymycin are derived from the 12-membered ring macrolactone 10-deoxymethynolide, while narbomycin and pikromycin are derived from the 14-membered ring macrolactone, narbonolide. The cloning and characterization of the biosynthetic gene cluster for these antibiotics reveals the key role of a type II thioesterase in forming a metabolic branch through which polyketides of different chain length are generated by the pikromycin multifunctional polyketide synthase (PKS). Immediately downstream of the PKS genes (pikA) are a set of genes for desosamine (des) biosynthesis and macrolide ring hydroxylation. The glycosyl transferase (encoded by desVIII) has the remarkable ability to catalyze glycosylation of both the 12- and 14-membered ring macrolactones. Moreover, the pikC-encoded P450 hydroxylase provides yet another layer of structural variability by introducing regiochemical diversity into the macrolide ring systems.

### Example 9

### Strategies Employing Modular PKS as PHA Monomer Providers

One strategy to exploit modular PKSs, e.g., modules of pikA or a FAS, to provide PHA monomers is to harvest polyketide intermediates as CoA derivatives using a TEII which is converted to an acyl-CoA transferase (mTEII). PikTEII is a small enzyme (281 amino acids) encoded by pikAV in S. venezuelae. The primary function of the wild-type enzyme is to catalyze the release of a polyketide chain at the fifth module in the pikA pathway as 10-deoxymethonolide. The enzyme most likely binds to the fifth module (PikAIIl) ACP (ACP₅) and releases the acyl chain attached to it. This relationship, TEII and its cognate ACP₅, can be exploited to produce a polyketide having different chain lengths by moving Pik ACP₅ to a different position in the cluster. For example, by moving ACP₅ into the second module in place of ACP₂, a triketide instead of hexoketide may be produced by the cluster. Further, moving KR5 together with ACP₅ into the second module, and replacing the DH, KR, and ACP domains, a 3-hydroxyl triketide is produced that is structurally suitable as PHA monomer. A mutant TEII (mTEII) catalyzes the release of the triketide as CoA form. The triketide-CoA, 3,5-dihydroxyl-4-methyl-heptonyl-CoA, is a substrate for PHA polymerase, e.g., PhaC1 from P. olivarus, which, in turn, can incorporate the monomer into a polymer.

A second strategy includes the harvesting of a polyketide intermediate as a CoA derivative using a TEI which has been converted to an acyl-CoA transferase (mTE). Thus, the second strategy for 3-hydroxyacyl-CoA monomer production is to exploit the TE domain (TEI) within the PKS module. It has been demonstrated that the TE domain can release polyketide intermediates attached to the ACP domain within the same module. Moving the TEI to a different position in a PKS cluster results in the production of a polyketide having a different chain length. Similarly, a mutant TEI (mTEI) (i.e., one which is an acyl-CoA transferase) releases the polyketide intermediate to acyl-CoA, which then is polymerized by PHA synthetase. Preferably, a mutant TE domain in the pikA4 gene cluster is moved into pik module 1, fusing it immediately downstream of ACP 1. The recombinant enzyme produces 2-(S)-methyl-3(R)-hydroxylveleratyl-CoA, which is a suitable substrate for PHA polymerase PhaC1. Therefore, the coexpression of the polymerase with the recombinant PKS produces a polymer.

A third strategy is to directly collect polyketide intermediates as substrates for PHA synthesis by fusing a PHA polymerase with a polyketide synthase. The first two strategies produce 3-hydroxylacyl-CoA as a substrate for PHA synthesis by employing a mutant PKS enzyme (TEI or TEII). As PHA polymerase may be active on acyl-ACP itself if the acyl-ACP is properly oriented, the third strategy fuses a PHA polymerase downstream of an ACP in a PKS protein. The PHA synthetase then serves as a domain within the chimeric multifunctional enzyme in place of a TE domain. The PKS portion of the protein catalyzes the synthesis of a 3-hydroxylacyl-ACP intermediate and then the PHA synthetase domain accepts it as substrate and adds the 3-hydroxylacyl monomer to the growing polyhydroxyalkanoate chain. The process regenerates ACP function so that the reaction can go on repeatedly to synthesize a PHA of multiple units. For example, a phaC1 gene is fused directly downstream of pik ACP1 so as to produce a chimeric enzyme that catalyzes the synthesis of a polymer.

The strategies described above can produce PHAs of complex structure, and having superior properties. In addition, the structure can be easily fine-tuned by modifying the PKS gene, thus resulting in PHAs having desired properties or functions.

### Example 10

### Control of Macrolactone Structure by Alternative Expression of a Modular Polyketide Synthase

Material and Methods

Media. Streptomyces venezuelae ATCC 15439 produces two groups of macrolide antibiotics: the 12-membered ring macrolides methymycin and neomethymycin, and the 14-membered ring macrolides pikromycin and narbomycin (FIG. 28). Methymycin and neomethymycin are derived from the 12-membered ring macrolactone 10-deoxymethynolide and are produced in SCM medium (Lambalot et al., 1992), whereas pikromycin and narbomycin are derived from the 14-membered ring macrolactone narbonolide and are produced in PGM medium (Xue et al., 1998).

Genetic Manipulation of S. venezuela. Mutant AX910 and AX912 were created by targeted gene replacement. The mutation plasmid pDHS910 was created by ligating two DNA fragments flanking the TE domain so that the TE domain was deleted and a hexa-histidine sequence was introduced at its position. The primer pairs that were used to amplified the flanking DNA in polymerase chain reaction (PCR) are 5′-CCCGAATTCGCCGCCGCCATGGCCGAA-3′ (SEQ ID NO:42) and 5′-GTGATGCATCGGCTCGGCGACGGCCCAGTTCCGCT-3′ (SEQ ID NO:43); and 5′-ATGCATCACCACCACCACCACTGAGGGGGCGGGCAAGTGACCGAC-3′ (SEQ ID NO:44) and 5′-GGGTCTAGAGCTGCACCGGCGGGTCGTAGCGGA-3′ (SEQ ID NO:45). Plasmid pDHS910 was introduced into S. venezuelae AX905 (Xue et al., 1998) which has a kanamycin resistance marker at the position of pikAV. Following procedures established by Xue et al. (1998), mutant AX910 (12 colonies) was isolated by screening for a kanamycin sensitive phenotype. The expected genotype of the mutant was confimned by genomic Southern hybridization. Mutation plasmid pDHS912 was generated by replacing a BamHI-BglII fragment (the DNA fragment corresponding to the pikA V gene immediately downstream of the TE domain) in pDHS910 with a kanamycin resistance gene (Denis et al., 1992). Thus, the TE domain as well as the TEII gene pikAV were disrupted in the mutant AX912. Plasmid pDHS912 was transferred into wild type S. venezuelae and mutant AX912 (12 colonies) was selected according to the procedures of Xue et al. (1998).

Western Blot Analysis. Western blot analysis of PikAIV followed standard procedures (Sambrook et al., 1989). The total protein of S. venezuelae AX910, AX912, or wild type was first prepared from a four-day culture in either SCM or PGM medium. The protein extract was separated on a 10% SDS-PAGE, transferred to PVDF membrane (Bio-Rad, Hercules, Calif.), hybridized with anti-6xHis antibody (Qiagen, Valencia, Calif.), and visualized using a secondary antibody conjugated to alkaline phophatase (Sigma, St. Louis, Mo.).

Construction of Complementation Plasmids. The pikA promoter, PpikA, was isolated as an EcoRV-EcoRI fragment between pikAI and pikRI in the pik cluster (Xue et al., 1998). To create a plasmid for complementation, a DNA fragment encoding PikAV was first PCR-amplified and placed downstream of the EcoRI site in such a way that PikAV was translationally coupled to the leader sequence of pikAI in PpikA to give plasmid pDHS702. Then, plasmids pDHS704, pDHS705, pDHS706, pDHS707, and pDHS708 were constructed by cloning various lengths of the pikAIV-pikAV region into pDHS702 replacing pikAV. The various lengths of pikAIV were PCR-amplified from cosmid pLZ51 (Xue et al., 1998) by the following primer pairs: prepared with primers 5′

| | |
|---|---|
| 5′-GAATTCATCGAGGGGGCGGGCAAGTGA-3′ | (SEQ ID NO:46) |
| and | |
| 5′-ATGCATCAGGTCGTCGGTCACCGTGGGTTCT- | (SEQ ID NO:47) |
| 3′ for pDHS7O2; | |
| 5′-GGATCCGCGCCGGGATGTTCCGCGCCCTGT-3′ | (SEQ ID NO:48) |
| and | |
| 5′-AAAATGCATCAGAGGTCTGTCGGTCACTTGC- | (SEQ ID NO:49) |
| 3′, for pDHS704; | |
| 5′-AAAAGATCTTGATGGTGCAGGCGCTGCGCCACG | (SEQ ID NO:50) |
| GGGTGCTG-3′ and | |
| 5′-AAAATGCATCAGAGGTCTGTCGGTCACTTGC- | (SEQ ID NO:49) |
| 3′ for pDHS708; and | |
| 5′-AAAAGATCTCCAACGAACAGTTGGTGGACGCT- | (SEQ ID NO:51) |
| 3′ and | |
| 5′-AAAATGCATCAGAGGTCTGTCGGTCACTTGC- | (SEQ ID NO:49) |
| 3′ for pDHS707. | |

The fragment in pDHS705 (EcoRI-BamH1) and pDHS706 (EcoRI-BglII) was isolated directly from restriction digestion of cosmid pLZ51 (Xue et al., 1998) and ligated into EcoRI-BglII treated pDHS702.

Antibiotic Extraction and Identification. Extraction, identification, and quantitation of methymycin and related compounds followed a procedure developed by Cane et al. (1993), which is summarized in Xue et al. (1998).

Results and Discussion

Deletion of the TE Domain from PikAIV. Production of both 10-deoxymethynolide and narbonolide is mediated by a single PKS cluster (pikA) in S. venezuelae (Xue et al., 1998). The pikA-encoded PKS is composed of PikAI, PikAII, PikAIII, and PikAIV (FIG. 28) multifunctional proteins similar to EryAI-AIII except that PikAIII and PikAIV each contain a single module in contrast to the bimodular EryAIII (Donadio et al., 1991). Moreover, PikAV is an independent thioesterase (TEII) that is distinct from the thioesterase domain (TE) located at the C-terminus of PikAIV. The modular organization of PikA indicates that PikAI-PikAIII produces a hexaketide that cyclizes into 10-deoxymethynolide, and that PikAI-PikAIV produces a heptaketide that cyclizes into narbonolide (FIG. 28). Termination of polyketide assembly at the heptaketide stage is likely catalyzed by the C-terminal TE domain in PikAIV, which is analogous to chain termination in the erythromycin pathway. However, it was not clear how the PikA system terminates polyketide assembly to produce the 12-membered ring aglycone, 10-deoxymethynolide. Genetic evidence excluded PikAV (TEH) as the determining factor in alternative termination since deletion of pikAV reduced the production of both macrolactones (Xue et al, 1998).

To study the role of PikAIV in alternative termination, two mutant strains of S. venezuelae were created in which PikAIV was disrupted by deleting the C-terminal thioesterase (TE) domain. In mutant AX910, an inframe deletion was engineered to remove the TE domain from S. venezuelae chromosome. In a second mutant, AX912, the TE domain as well as the downstream TEII gene (pikAV) was removed from the bacterial chromosome. As expected, S. venezuelae AX912 is devoid of antibiotic production since the mutant lacks the thioesterase activities that are necessary to release the polyketide chain from the Pik PKS protein. It was expected that the AX910 mutant strain would at least produce the 12-membered ring macrolides methymycin and neomethymycin because the sixth condensation cycle catalyzed by PikAIV is not required for 10-deoxymethynolide formation. Surprisingly, mutant AX910 produced trace amounts of pikromycin, however, methymycin and neomethymycin were completely absent from the fermentation broth. Since the AX910 mutant contains an inframe deletion of the pikAIV-encoded TE domain, the potential for a downstream polar effect (on the pikAV-encoded TEII enzyme) was avoided. This result suggested that PikAIV, or at least the TE domain within PikAIV, is involved directly in the production of the 12- as well as 14-membered ring macrolactones.

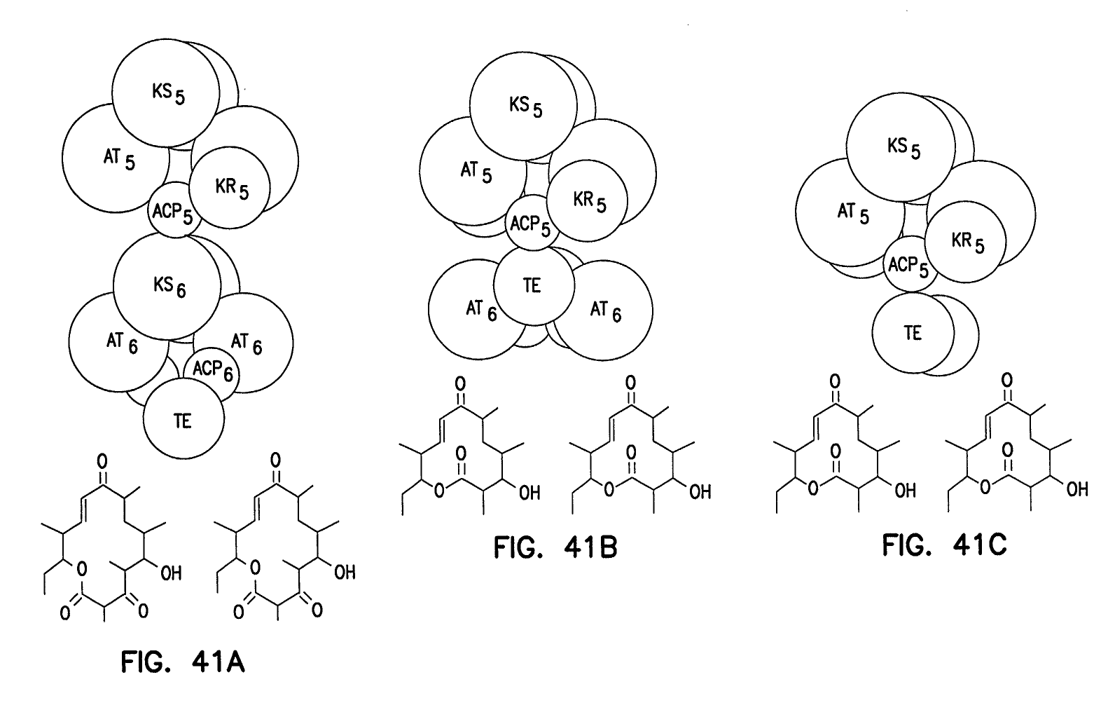

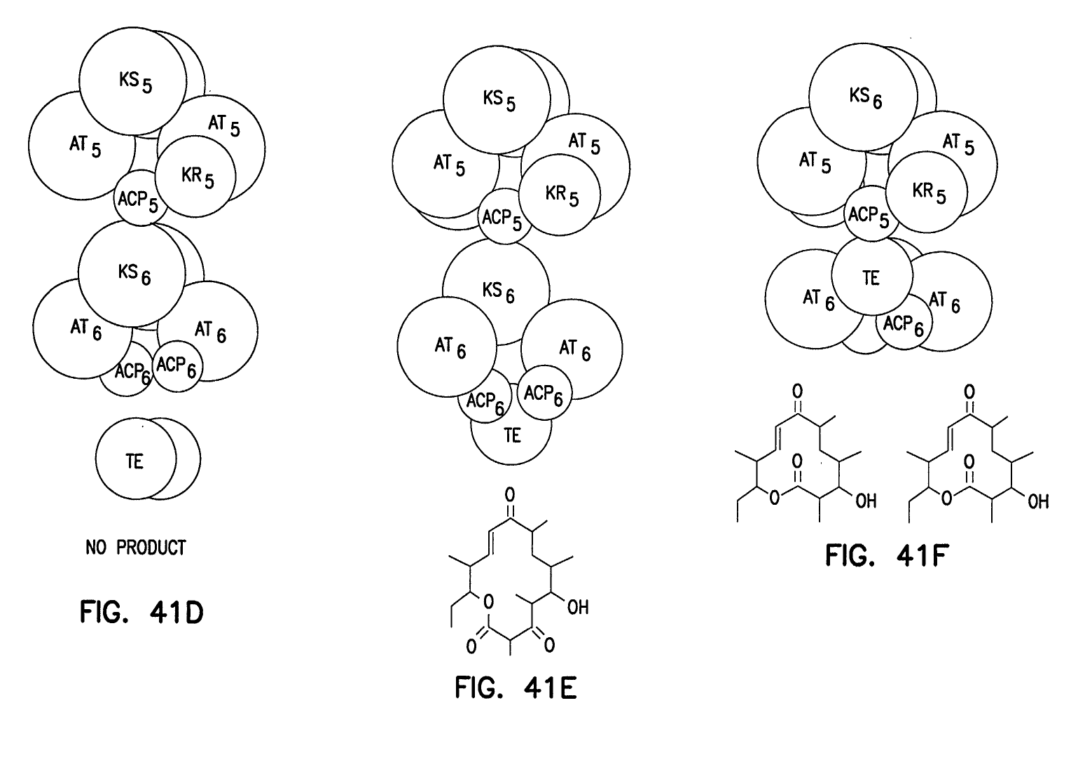

Probing the expression of PikAIV. To investigate the differential expression of pikAIV using culture conditions for methymycin (SCM medium) or pikromycin (PGM medium) production, the PikAIV protein was first tagged by a hexa-histidine sequence replacing the TE domain at its C-terminus. Expression of PikAIV was then probed with anti-6×His antibody in a Western blot that revealed a single protein band under conditions for either methymycin or pikromycin production in the mutant strains (AX910 and AX912). Interestingly, the protein detected from cell extracts obtained under culture conditions for methymycin production (SCM medium) was approximately 25 kDa lower in molecular weight compared to the protein detected under conditions for pikromycin production (PGM medium). The molecular weight of the protein detected under pikromycin culture conditions is 110 kDa, which is consistent with the predicted TE-truncated (6×His-tag replaced) form of PikAIV. Therefore, the protein detected under conditions for methymycin production must be an N-terminal truncated form of PikAIV (FIG. 41). Indeed, two potential alternative translation start sites have been located in the pikAIV sequence, with either predicted to generate the truncated form of PikAIV. The presumed alternative expression of pikAIV creates a protein product that contains only half of the Pik module 6 KS (KS₆) domain (FIG. 41). This result immediately pointed to a mechanism for alternative termination in the PikA system. Since the KS₆ domain is responsible for the condensation of the final extender unit, a PKS that is unable to catalyze this reaction could only produce the 12-membered ring macro lactone.

Complementation analysis of PikAIV. To investigate the functioning of the truncated form of PikAIV, the contribution of various domains in the multifunctional protein was tested by genetic complementation of S. venezuelae mutant strain AX912. An SCP2* -based low copy number plasmid (Lydiate et al., 1985) was designed and the target gene (comprised of alternative-length forms of pikAIV) was placed under the control of the native pikA promoter (Xue et al., 1998). Using this system, the expression of pikAIV from the plasmid would most closely resemble its normal temporal expression profile, and would also be synchronized with expression ofthe pikA cluster encoded on the S. venezuelae chromosome. This system was used to test the ability of alternative forms of the pikAIV-pikAV region (FIG. 41) to complement the TE-TEII double mutant strain AX912.

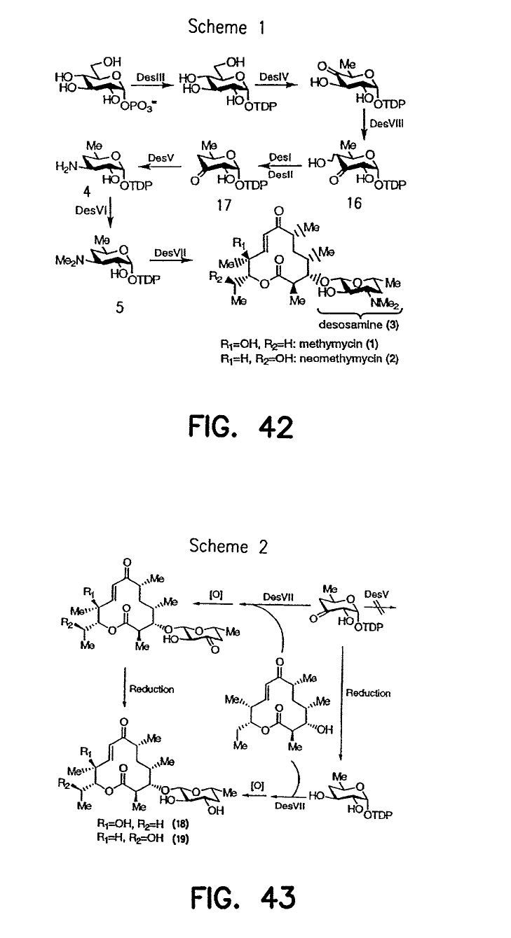

The results clearly demonstrated that the TE domain in PikAIV is critical for 10-deoxymethynolide formation. Specifically, all of the plasmid constructs that contain the TE domain including, pDHS704 (TE alone), pDHS705 (ACP₆-TE), pDHS706 (ACP₆-TE::TEII), pDHS708 (AT₆-ACP₆-TE), and pDHS707 (KS₆-AT₆-ACP₆-TE), complemented mutant AX912 to give 10-deoxymethynolide. Interestingly, other domains in the truncated form of PikAIV, especially the AT domain, were necessary for effective production of 10-deoxymethynolide. The most efficient production of 10-deoxymethynolide resulted from complementation by pDHS708 (AT₆-ACP₆-TE), which contains the AT domain and closely mimics the truncated form of PikAIV detected in wild type S. venezuelae under conditions for methymycin production (FIG. 41). The relatively efficient complementation by the TE domain alone (pDHS704) leading to 10-deoxymethynolide is especially intriguing and may result from two possible (or one of the two) complementation scenarios. Specifically, it may involve interaction of the TE domain directly with PikAIII (FIG. 42C) and/or formation of a wild type-like PKS complex (FIG. 42B) by the TE domain expressed from the plasmid interacting with the rest of PikAIV (expressed from the corresponding AX912 chromosomal allele) through noncovalent interactions.

Interestingly, the TE domain alone did not complement AX912 (TE-TEII double mutant) to give narbonolide production (FIG. 41). This is consistent with a recent result (Gokhale et al., 1999) obtained from the erythromycin PKS system suggesting that the TE domain may not interact significantly with it natural endogenous module (e.g., EryAIII or PikAIV) but must be covalently linked to be functional. However, the failure to complement may be due in part to introduction of the hexa-histidine at the C-terminus of the engineered PikAIV protein in AX912. Interestingly, pDHS708 (AT₆-ACP₆-TE) did complement AX912 under culture conditions for pikromycin production resulting in equal amounts of 10-deoxymethynolide and narbonolide (FIG. 41). This product pattern occurs due to formation of hetero- and homodimeric structures of PikAIV as shown in FIG. 42E and FIG. 42F, respectively. These results are in accord with a model in which an N-terminal truncated form of PikAIV is responsible for 10-deoxymethynolide formation while expression of full-length PikAIV is responsible for narbonolide production.

Comparing the complementation of pDHS705 (ACP₆-TE) and pDHS706 (ACP₆-TE::TEII) further revealed the activity of pik TEII. Although TEII alone is not sufficient for polyketide termination (as shown in pDHS702 complementation, see FIG. 41), the independent thioesterase did enhance the production of both 10-deoxymethynolide and narbonolide (FIG. 41). Particularly in the case of narbonolide formation, the presence of TEII in pDHS706 (ACP₆-TE::TEII) complementation helped to boost polyketide production to a level that was otherwise undetectable in AX912 (pDHS705 (ACP₆-TE)). This accessory role of TEII is consistent with previous observations in the pikromycin system (Xue et al., 1998), as well as with other PKS (Rangaswamy et al., 1998) and non-ribosomal peptide synthetase (NRPS) systems (Schneider et al., 1998).

Mechanistic Models for the Alternative Termination by PikAIV. The complementation experiments described above strongly suggest that TE is the key enzymatically active domain in the truncated PikAIV polypeptide, although the entire protein (including AT, ACP, TE, and probably a partial KS domain) is much more effective for polyketide production. A structural model based on the proposed helical form of the erythromycin PKS complex (Staunton et al., 1996) was developed to illustrate the role of PikAIV in alternative termination in the pik-encoded PKS. Under conditions for pikromycin production, wild type S. venezuelae expresses a full length PikAIV module, which interacts with PikAIII and elongates the growing polyketide chain on ACP₅ by adding a methylmalonate unit (the activity of KS₆) to ultimately produce the 14-membered ring macrolactone, narbonolide (FIG. 42A). On the other hand, the truncated form of PikAIV that lacks KS₆ is expressed under culture conditions for methymycin production. The molecular space left unoccupied by KS₆ truncation is then presumably filled by the TE domain that would be aligned to interact directly with ACP₅ to release the 12-membered ring macrolactone (FIG. 42B). In both cases, the main part of PikAIV is predicted to remain fixed. A small movement of the TE domain into the unoccupied space (left by KS₆ truncation) would result in the bypass of the AT₆-ACP₆ catalytic domains in the truncated PikAIV, while retaining thioesterase activity. Evidently, the main function of truncated PikAIV is to serve as a scaffold that orients the TE domain and stabilizes the interacting complex between PikAIiU and PikAIV, therefore, greatly increasing the production of 10-deoxymethynolide.

Efficient production of 10-deoxymethynolide by a truncated form of PikAIV suggests that the AT, rather than the KS domain plays a pivotal role in the structure and function of modular PKS. The KS₆-truncated form of PikAIV generated from the pDHS708 (AT₆-ACP₆-TE) complementation plasmid probably forms a heterodimer with the product of the corresponding AX912 chromosomal allele to generate narbonolide (FIG. 42E), and it also efficiently forms a homodimer to produce 10-deoxymethynolide (FIG. 42F). However, this dimerization capacity was severely limited when the AT₆ domain was truncated in pDHS705 (ACP₆-TE). Furthermore, the complete absence of complementation by pDHS704 (TE alone) to give narbonolide (under culture conditions for pikromycin production) suggests that a dominant interaction exists between KS₆ and PikAII (FIG. 42D), which may be the primary basis of module-module recognition and docking in multifunctional PKS systems. The pikA system in S. venezuelae provides a unique opportunity as well as a powerful tool to study these fundamental interactions in further detail.

It is valuable to compare alternative termination by differential expression of PikAIV in S. venezuelae with engineered polyketide chain-length manipulations from other PKS systems. In the erythromycin PKS, the TE domain from EryAII was moved to upstream domains and covalently linked to alternative ACPs resulting in truncated polyketides (Cortes et al, 1995; Kao et al., 1995). In each case, the capacity for producing the full-length polyketide product was subsequently eliminated. In contrast, by linking the TE domain of PikAIV to an upstream module by protein-protein interactions, S. venezuelae retains the capacity to generate two alternative-sized macrolactones. Sequence analysis (Xue et al., 1998) suggested that the pikA may have evolved from a six-module PKS that generated a 14-membered ring macrolactone. It is, therefore, interesting to consider that the structural and regulatory evolution of pikA to produce the rare 12-membered ring macrolactone may be the result of endogenous genetic selection to overcome antibiotic resistance within the ecological milieu of the antibiotic producing microorganism. The pikA system provides a natural example of a branched metabolic pathway with the capacity to generate multiple macrolactone systems that may be readily exploited for combinatorial biosynthetic creation of novel natural products.

### Example 11

A mutant of S. venezuelae (KdesV-41) was constructed that had the desV gene disrupted (Zhao et al., J. Am. Chem. Soc., 120, 12159 (1998)). Since desV encodes the 3-aminotransferase that catalyzes the conversion of the 3-keto sugar 17 (FIG. 42) to the corresponding amino sugar 4, deletion of this gene should prevent C-3 transamination, resulting in the accumulation of 17. It was expected that if the glycosyltransferase (DesVII) of this pathway is capable of recognizing and processing the keto sugar intermediate 17, the macrolide product(s) produced by the KdesV-41 mutant should have an attached 3-keto sugar. Surprisingly, the two products isolated were the methymycin/neomethymycin analogues 18 and 19, each carrying a 4,6-dideoxyhexose (FIG. 43). While this result demonstrated a relaxed specificity for the glycosyltransferase toward its sugar substrate, it also indicated the existence of a pathway-independent reductase in S. venezuelae that can stereospecifically reduce the C-3 keto group of the sugar metabolite.

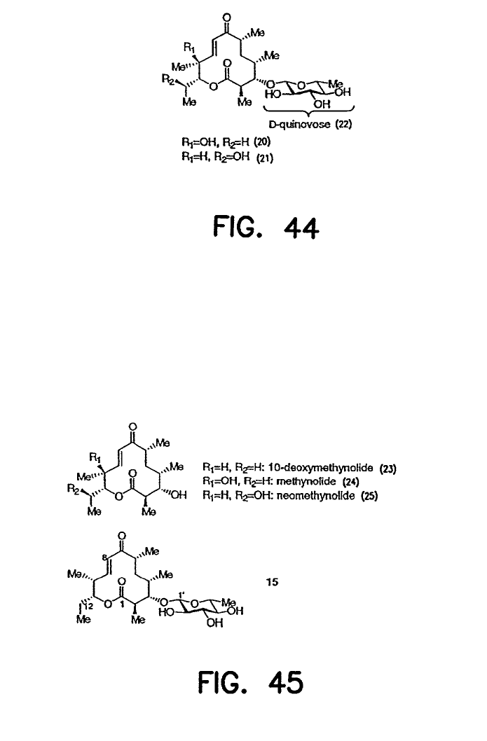

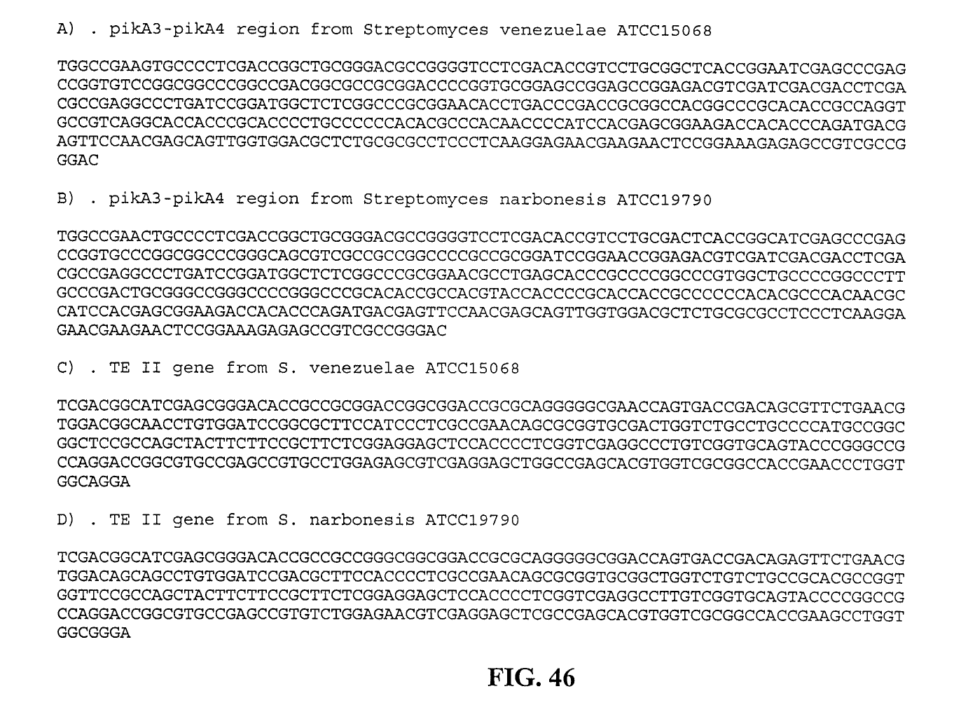

To explore the possibility of generating a mutant capable of synthesizing new macrolides of this class containing an engineered sugar, the desi gene, which has been proposed to encode the dehydrase responsible for the C-4 deoxygenation in the biosynthesis of desosamine, was altered with the prediction that it would lead to the incorporation of D-quinovose (22; FIG. 44), also known as 6-deoxy-D-glucose, into the final product(s). The rationale was based on the following: (1) Desosamine biosynthesis will be “terminated” at the C-4 deoxygenation step due to desI deletion and, thus, should result in the accumulation of 3-keto-6-deoxyhexose 16 (FIG. 42). (2) By taking advantage of the existence of a 3-ketohexose reductase in S. venezuelae, the sugar intermediate 15 is expected to be reduced stereospecifically to D-quinovose (22). (3) The glycosyltransferase (DesVII), with its relaxed specificity toward the sugar substrate, should catalyze the coupling of 22 to the macrolactones to give new macrolides 20 and 21 containing the engineered sugar D-quinovose (FIG. 44).

A disruption plasmid, pDesI-K, derived from pKC1139 that contains an apramycin resistant marker, was constructed in which desi was replaced by the neomycin resistance gene, which also confers resistance to kanamycin. This construct was then introduced into wild type S. venezuelae by conjugal transfer using Escherichia coli S17-1 as the donor strain (Bierman et al., 1992). Several double crossover mutants were identified on the basis of their phenotypes of kanamycin resistant (Kan^{R}) and apramycin sensitive (Apr^{S}). One mutant, KdesI-80, was selected and grown at 29° C. in seed medium (100 mL) for 48 hours and then inoculated and grown in vegetative medium (5 L) for another 48 hours (Cane et al., 1993). The fermentation broth was centrifuged to remove cellular debris and mycelia, and the supernatant was adjusted to pH 9.5 with concentrated potassium hydroxide solution. The resulting solution was extracted with chloroform, and the pooled organic extracts were dried over sodium sulfate and evaporated to dryness. The yellow oil was subjected to flash chromatography on silica gel using a gradient of 0-12% methanol in chloroform, and the isolated products were further purified by HPLC using a C₁₈ column eluted isocratically with 50% acetonitrile in water. As expected, no methymycin or neomethymycin was detected; instead, 10-deoxymethynolide 23 was found as the major product (approximately 600 mg). Significant quantities of methynolide 24 (approximately 40 mg) and neomethynolide 25 (approximately 2 mg) were also isolated (FIG. 45). A new macrolide 15 containing D-quinovose (3.2 mg) was produced by this mutant. Its structure was fuilly established by spectral analyses. Spectral data (J values are in hertz) for 15: ¹H NMR (CDCl₃) 67 6.76 (1H, dd, J=16.0, 5.5, 9-H), 6.43 (1H, d, J=16.0, 8-H), 4.97 (1H, ddd, J=8.4, 5.9, 2.5, 11-H), 4.29 (1H, d, J=8.0, 1′-H), 3.62 (1H, d, J=10.5, 3-H), 3.49 (1H, t, J=9.0, 3′-H), 3.36 (1H, dd, J=9.0, 8.0, 2′-H), 3.32 (1H, dq, J=8.5, 5.5, 5′-H), 3.23 (1H, dd, J=9.0, 8.5, 4′-H), 2.82 (1H, dq, J=10.5, 7.0, 2-H), 2.64 (1H, m, 10-H), 2.55 (1H, m, 6-H), 1.70 (1H, m, 12a-H), 1.66 (1H, bt, J=12.5, 5b-H), 1.56 (1H, m, 12b-H), 1.40 (1H, dd, J=12.5, 4.5, 5a-H), 1.35 (3H, d, J=7.0, 2-Me), 1.31 (3H, d, J=5.5, 5′-Me), 1.24 (1H, bdd, J=10.0, 4.5, 4-H), 1.21 (3H, d, J=7.0, 6-Me), 1.11 (3H, d, J=6.5, 10-Me), 1.00 (3H, d, J=7.0, 4-Me), 0.92 (3H, t, J=7.5, 12-Me); ¹³C NMR (CDCl₃) 67 205.0 (C-7), 174.7 (C-1), 146.9 (C-9), 125.9 (C-8), 102.9 (C-1′), 85.4 (C-3), 76.5 (C-3′), 75.5 (C-4′), 74.7 (C-2′), 73.9 (C-11), 71.6 (C-5′) 45.0 (C-6), 43.9 (C-2), 37.9 (C-10), 34.1 (C-5), 33.4 (C-4), 25.2 (C-12), 17.7 (6-Me), 17.5 (5′-Me), 17.4 (4-Me), 16.2 (2-Me), 10.3 (12-Me), 9.6 (10-Me); high-resolution FAB-MS calculated for C₂₃H₃₈O₈ (M+H)⁺443.2644, found 443.2661.

The fact that macrolide 15 containing D-quinovose is indeed produced by the desI mutant is significant. First, the formation of quinovose as predicted further corroborates the presence of a pathway-independent reductase in S. venezuelae that reduces the 3-keto sugars. Interestingly, this reductase is able to act on the 4,6-dideoxy sugar 17 as well as the 6-deoxy sugar 16, suggesting that it is oblivious to the presence of a hydroxyl group at C-4. However, it is not clear at this point whether the reduction occurs on the free sugar or after it is appended to the aglycone. Second, the retention of the 4-OH in quinovose as a result of desI deletion provides strong evidence supporting the assigned role of desi to encode a C-4 dehydrase. Moreover, the results again show that the glycosyltransferase (DesVII) of this pathway can recognize alternative sugar substrates whose structures are considerably different from the original amino sugar substrate desosamine. While the incorporation of quinovose is important, another noteworthy, albeit unexpected, result was the fact that the aglycone of the isolated macrolide 15 was 10-deoxy-methynolide 23 instead of methynolide 24 and neomethynolide 25. It is possible that the cytochrome P450 hydroxylase (PikC), which catalyzes the hydroxylation of 10-deoxy-methynolide at either its C-10 or C-12 position (Xue et al., Chem. Biol., 5, 661 (1998)), is sensitive to structural variations in the appended sugar. It could be argued that the presence of the 4-OH group in the sugar moiety is somehow responsible for decreasing or preventing hydroxylation of the macrolide.

Thus, the results demonstrate the feasibility of combining pathway-dependent genetic manipulations and pathway-independent enzymatic reactions to engineer a sugar of designed structure. It is conceivable that the pathway-independent enzymes could also be used in concert with the natural biosynthetic machinery to generate further structural diversity, which can provide an array of random compounds.

### REFERENCES

Andersen, J. R., Hutchinson, C. R. J. Bacteriol., 174:725-735 (1992).

Aparicio, J. F., Molnar, I., Schwecke, T., Konig, A., Haydock, S. F., Khaw, L. E., Staunton, J., Leadlay, P. F. Gene, 169:9-16 (1996).

Arisawa, A., Kawamura, N., Takeda, K., Tsunekawa, H., Okamura, K., Okamoto, R. Appl. Environ. Microbiol., 60:2657-2660 (1994).

August, P. R., Tang, L., Yoon, Y. J., Ning, S., Muller, R., Yu, T. W., Taylor, M., Hoffmnann, D., Kim, C. G., Zhang, X., Hutchinson, C. R. & Floss, H. G. Chem. Biol., 5:69-79 (1998).

Baltz, R. H., Seno, E. T. Annu. Rev. Microbiol., 42:547-574 (1988).

Bibb, M. J., Bibb, M. J., Ward, J. M., Cohen, S. N. Mol. Gen. Genet., 199:26-36 (1985).

Bierman, M., Logan, R., O'Brien, K., Seno, G., Nagaraja, R., Schoner, B. E. Gene, 116:43-49 (1992).

Box, R. P. Clin. Infect. Dis., 24:S151 (1997).

Cane, D. E., Lamnbalot, R. H., Prabhakaran, P. C., Ott, W. R. J. Am. Chem. Soc., 115:522-526 (1993).

Carreras, C. W., Pieper, R., Khosla, C. In Bioorganic Chemistry Deoxysugars, Polyketides & Related Classes: Synthiesis, Biosynthesis, Enzymes, Rohr, J. (ed.), Springer:Berlin, 85-126 (1997).

Castle, L. A., Smith, K. D., Morris, R. O. J. Bacteriol., 174:1478-1486 (1992).

Celmer, W. D., Nagel, A. A., Wadlow, J. W., Tatematsu, H., Ikenaga, S., Nakanishi, S. Abstracts of Papers of 24th Intersci. Conf. on Antimicrob. Agents Chemother., No. 1142, Washington, D.C. (1985).

Cortes, J. Haydock, S. F., Roberts, G. A., Bevitt, D. J., Leadlay, P. F. Nature, 348:176-8 (1990).

Cortes, J., Wiesmann, K. E., Roberts, G. A., Brown, M. J., Staunton, J., Leadlay, P. F. Science, 268:1487-9 (1995).

Cundliffe, E. C. Annu. Rev. Microbiol., 43:207-233 (1989).

Cundliffe, E. Antimicrob. Agents Chemother., 36:348-352 (1992).

Davies, J. Nature, 383:219-220 (1996).

Denis, F., Brzezinski, R. Gene, 111:115-118 (1992).

Djerassi, C., Zderic, J. A. J. Am. Chem. Soc., 78:6390-6395 (1956).

Donadio, S., McAlpine, J. B., Sheldon, P. J., Jackson, M., Katz, L. Proc. Natl. Acad. Sci, U.S.A., 90:7119-7123 (1993).

Donadio, S., Staver, M. J., McAlpine, J. B., Swanson, S. J., Katz, L. Science, 252:675-9 (1991).

Donadio, S., Katz, L. Gene, 111:51-60 (1992).

Donin, M. N., Pagano, J., Dutcher, J. D., McKee, C. M. Antibiotics Annu., 1:179-185 (1953-1954).

Epp, J., Huber, M. L. B., Tuner, J. R., Goodson, T., Schoner, B. E. Gene, 85:293-301 (1989).

Flinn, E. H., Sigal, M. V., Jr., Wiley, P. F., Gerzon, K. J. Am. Chem. Soc., 76:3121-3131(1954).

Gaisser, S., Bohm, G. A., Cortes, J., Leadlay, P. F. Mol. Gen. Genet., 256:239-251 (1997).

Gandecha, A. R., Large, S. L., Cundliffe, E. Gene, 184:197-203 (1997).

Geistlich, M., Losick, R., Turner, J. R., Rao, R. N. Mol. Microbiol., 6:2019-2029 (1992).

Gokhale, R. S., Hunziker, D., Cane, D. E., Khosla, C. Chem. Biol., 6:117-125 (1999).

Haydock, S. F., Dowson, J. A., Dhillon, N., Roberts, G. A., Cortes, J., Leadlay, P. F. Mol. Gen. Genet., 230:120-128 (1991).

Hernandez, C., Olano, C., Mendez, C., Salas, J. A. Gene, 134:139-140 (1993).

Hopwood, D. A., Sherman, D. H. Annu. Rev. Genet., 24:37-66 (1990).

Hopwood, D. A., Malpartida, F., Kieser, H. M., Ikeda, H., Duncan, J., Fujii, I., Rudd, B. A., Floss, H. G., Omura, S. Nature, 314:642-644 (1985).

Hopwood, D. A., Bibb, M. J., Chater, K. J., Kieser, T., Bruton, C. J., Kieser, H. M., Lydiate, D. J., Smith, C. P., Ward, J. M., Schrempf, H., Genetic Manipulation of Streptomyces: A Laboratory Manual (The John Innes Foundation) (1985).

Hori et al., Chem. Comm., 304 (1971).

Hutchinson, C. R., Fujii, I. Annu. Rev. Microbiol., 49:201-238 (1995).

Ingrosso, D., Fowler, A. V., Bleibaum, J., Clarke, S. J. Biol. Chem., 264:20130-20139 (1989).

Jacobsen, J. R., Hutchinson, C. R., Cane, D. E., Khosla, C. Science, 277:367-369 (1997).

Jenksins, G., Cundliffe, E. Gene, 108, 55-62 (1991).

Kakavas, S. J., Katz, L., Stassi, D. J. Bacteriol., 179:7515-22 (1997).

Kao, C.M., Luo, G.L., Katz, L., Cane, D.E., Khosla, C. J. Am. Chem. Soc., 117:9105-9106 (1995).

Katz, L., Donadio, S. Annu. Rev. Microbiol., 47:875-912 (1993).

Katz, L., Chem. Rev., 97:2557-2575 (1997).

Khosla, C., Chemn. Rev., 97:2577-2590 (1997).

Khosla, C., Zawada, R. J. Trends Biotechnol., 14:335-341 (1996).

Kirschning, A., Bechthold, A. F. -W., Rohr, J. In Bioorganic Chemistry Deoxysugars, Polyketides & Related Classes: Synthesis, Biosynthesis, Enzymes, Rohr, J. (ed.), Springer:Berlin 1-84 (1997).

Kramer, P. J., Khosla, C. Annu. N.Y. Acad. Sci., 799:32-45 (1996).

Kuo, M.-S., Chirby, D. G., Argoudelis, A. D., Cialdella, J. I., Coats, J. H., Marshall, V. P. Antimicrob. Agents Chemother., 33:2089-2091 (1989).

Lambalot, R. H., Cane, D. E. J. Antibiot., 45:1981-1982 (1992).

Lin, E. C. C., Goldstein, R., Syvanen, M. Bacteria, Plasmids, and Phages, An Introduction to Molecular Biology, Harvard University Press:Cambridge, p. 123 (1984).

Liu, H. -w., Thorson, J. S. Annu. Rev. Microbiol., 48:223-256 (1994).

Lydiate, D. J., Malpartida, F., Hopwood, D. A. Gene, 35:223-235 (1985).

Madduri, K., Kennedy, J., Rivola, G., Inventi-Solari, A., Filippini, S., Zanuso, G., Colombo, A. L., Gewain, K. M., Occi, J. L., MacNeil, D. J., Hutchinson, C. R. Nature Biotech., 16:69-74 (1998).

Mangahas, F.R. MS Thesis, University of Minnesota, 1996.

Marahiel, M. A., Stachelhaus, T., Mootz, H. D., Chem. Rev., 97:2651-2673 (1997).

Marsden, A. F. A., Wilkinson, B., Cortes, J., Dunster, N. J., Staunton, J., Leadlay, P. F. Science, 279:199-201 (1998).

Merson-Davies, L. A., Cundliffe, E. Mol. Microbiol., 13:349-355 (1994).

Merson-Davies, L. A., Cundliffe, E. Mol. Microbiol., 13:347-355 (1994).

Motamedi, H., Cai, S. J., Shafiee, A., Elliston, K. O. Eur. J. Biochem., 244:74-80 (1997).

Muth, G., Nubhaumer, B., Wohlleben, W., Puhler, A. Mol. Gene. Genet., 219:341-348 (1989).

Niemi, J., Mantsala, P. J. Bacteriol., 177:2942-2945 (1995).

Omura, S. (ed.) Macrolide Antibiotics, Chemistry, Biology, and Practice, Academic Press:New York (1984).

Omuras et al., J. Antibio., 29, 316 (1971).

Rangaswamy, V., Mitchell, R., Ullrich, M., Bender, C. J. Bacteriol., 180:3330-3338 (1998).

Sambrook, J., Fritsch, E. F., Maniatis, T. Molecular Cloning: A Laboratory Manual (Cold Spring Harbor Laboratory Press), 2nd edition (1989).

Sasaki, J., Mizoue, K., Morimoto, S., Omura, S. J. Antibiotics, 49:1110-1118 (1996).

Schneider, A., Marahiel, M. A., Arch. Microbiol., 169:404-410 (1998).

Schupp, T., Toupet, C., Cluzel, B., Neff, S., Hill, S., Beck, J. J., Ligon, J. M., J. Bacteriol., 177:3673-9 (1995).

Schwecke, T., Aparicio, J. F., Molnar, I., Konig, A., Khaw, L. E., Haydock, S. F., Oliynyk, M., Caffrey, P., Cortes, J., Lester, J. B., et al. Proc. Natl. Acad. Sci. U.S.A., 92:7839-7843 (1995).

Seo, S., Tomita, Y., Tori, K., Yoshimura, Y. J. Am. Chem. Soc., 100:3331-3339 (1978).

Service, R. F. Science, 270:724-727 (1995).

Stassi, D., Donadio, S., Staver, M. J., Katz, L. J. Bacteriol., 175:182-189 (1993).

Staunton, J., Caffrey, P., Aparicio, J. F., Roberts, G. A., Bethell, S. S., Leadlay, P. F. Nat. Struct. Biol., 3:188-192 (1996).

Staunton, J., Wilkinson, B., Chem. Rev., 97:2611-2629 (1997).

Summers, R. G., Donadio, S., Staver, M. J., Wendt-Pienkowski, E., Hutchinson, C. R., Katz, L. Microbiology, 143:3251-3262 (1997).

Swan, D. G., Rodriguez, A. M., Vilches, C., Mendez, C., Salas, J. A. Mol. Gen. Genet., 242:358-362 (1994).

Tuan, J. S., Weber, J. M., Staver, M. J., Leung, J. O., Donadio, S., Katz, L. Gene, 90:21-29 (1990).

Vilches, C., Hernandez, C., Mendez, C., Salas, J. A. J. Bacteriol., 174:161-165 (1992).

von Heijne, G. Nucleic Acids Res., 14:4683-4690 (1986).

von Heijne, G., Abrahmsen, L. FEBS Lett., 244:439-446 (1989).

Weber, J. M., Leung, J. O., Swanson, S. J., Idler, K. B., McAlpine, J. B. Science, 252:114-117 (1991).

Xue, Y., Zhao, L., Liu, H. -w., Sherman, D. H. Proc. Natl. Acad. Sci. U.S.A., 95: 12111-12116 (1998).

The complete disclosure of all patents, patent documents and publications cited herein are incorporated herein by reference as if individually incorporated. The foregoing detailed description and examples have been given for clarity of understanding only. No unnecessary limitations are to be understood therefrom. The invention is not limited to the exact details shown and described for variations obvious to one skilled in the art will be included within the invention defined by the claims.

What is claimed is:

1. An isolated and purified nucleic acid segment comprising a nucleic acid sequence comprising a desosamine biosynthetic gene cluster, a fragment or a biologically active variant thereof, wherein the nucleic acid sequence is not derived from the eryC gene cluster of Saccharopolyspora erythraea or Streptomyces antibioticus.

2. The isolated and purified nucleic acid segment of claim 1 comprising SEQ ID NO:3.

3. The isolated and purified nucleic acid segment of claim 1 which encodes DesI, DesII, DesIII, DesIV, DesV, DesVI, DesVII, DesVIII or DesR.

4. The isolated and purified nucleic acid segment of any one of claims 1 to 3 which is from Streptomyces venezuelae.

5. An expression cassette comprising the nucleic acid segment of any one of claims 1 to 4 operably linked to a promoter functional in a host cell.

6. A recombinant bacterial host cell in which at least a portion of a nucleic acid sequence encoding desosamine is disrupted so as to alter desosamine synthesis, wherein the nucleic acid sequence which is disrupted is not derived from the eryC gene cluster of Saccharopolyspora erythraea.

7. The host cell of claim 6 wherein the nucleic acid sequence which is disrupted encodes DesI, DesII, DesIII, DesIV, DesV, DesVI, DesVII, DesVIII or DesR.

8. A host cell, the genome of which is augmented with the expression cassette of claim 5.

9. A product produced by the host cell of any one of claims 6 to 8 which is not produced by the corresponding non-recombinant or non-augmented host cell.

10. The product of claim 9 which comprises a macrolide.

11. The product of claim 9 or 10 which is biologically active.

12. An isolated and purified nucleic acid segment comprising a nucleic acid sequence comprising a macrolide biosynthetic gene cluster encoding polypeptides which synthesize methymycin, pikromycin, neomethymycin, narbomycin, or a combination thereof, or a biologically active variant or fragment thereof.

13. The isolated and purified nucleic acid segment of claim 12 comprising SEQ ID NO:5.

14. The isolated and purified nucleic acid segment of claim 12 comprising a biologically active variant or fragment of SEQ ID NO:5.

15. The isolated and purified nucleic acid segment of claim 12 which encodes PikR1, PikR2, PikAI, PikAII, PikAIII, PikAIV, PikAV, PikC or PikD.

16. The isolated and purified nucleic acid segment of any one of claims 12 to 15 which is from Streptomyces venezuelae.

17. A host cell, the genome of which is augmented with the nucleic acid segment of any one of claims 12 to 16.

18. An isolated and purified nucleic acid sequence comprising SEQ ID NO:3, SEQ ID NO:5, a fragment thereof, the complement thereto, or which hybridizes thereto.

19. An isolated polypeptide encoded by the nucleic acid segment of any one of claims 1 to 4 or 12 to 16.

20. A recombinant host cell in which a pikAI gene, a pikAII gene, a pikAIII gene, a pikAIV gene, a pikB gene cluster, a pikAV gene cluster, a pikC gene, a pikR1 gene, a pikR2 gene, or a combination thereof, is disrupted so as to alter production of methymycin, neomethymycin, pikromycin, narbomycin, or a combination thereof.

21. A macrolide or polyketide product produced by the host cell of claim 17 or 20 which is not produced by the corresponding non-recombinant or non-augmented host cell.

22. The macrolide or polyketide of claim 21 which is biologically active.

23. An isolated and purified DNA molecule comprising a first DNA segment encoding a first module and a second DNA segment encoding a second module, wherein the DNA segments together encode a recombinant polyhydroxyalkanoate monomer synthase, and wherein at least one DNA segment is derived from the pikA gene cluster of Streptomyces venezuelae.

24. A method of providing a polyhydroxyalkanoate monomer, comprising:
(a) introducing into a host cell a DNA molecule comprising a DNA segment encoding a recombinant polyhydroxyalkanoate monomer synthase operably linked to a promoter functional in the host cell, wherein the recombinant polyhydroxyalkanoate monomer synthase comprises a first module and a second module, and wherein at least one DNA segment is derived from the pikA gene cluster of Streptomyces venezuelae; and
(b) expressing the DNA encoding the recombinant polyhydroxyalkanoate monomer synthase in the host cell so as to generate a polyhydroxyalkanoate monomer.

25. A recombinant vector comprising one or more modules of a polyketide synthase wherein at least one module is from Streptomyces venezuelae.

26. The method of claim 24 wherein the first module encodes a fatty acid synthase.

27. A method of providing a polyhydroxyalkanoate monomer, comprising:
(a) introducing into a host cell a DNA molecule encoding a fuision polypeptide, wherein the DNA molecule comprises a first DNA segment operably linked to a promoter functional in the host cell and a second DNA segment, wherein at least one DNA segment is derived from the pikA gene cluster of Streptomyces venezuelae; and
(b) expressing the DNA in the host cell so as to generate the fusion polypeptide.

28. The host cell of claim 8 or 17 the native genome of which does not comprise an intact macrolide biosynthetic gene cluster encoding polypeptides which synthesize methymycin, pikromycin, neomethymycin, or narbomycin.

29. A recombinant bacterial host cell comprising a deletion of the thioesterase domain of pikeAIV gene.

30. The recombinant host cell of claim 29 further comprising a deletion in the pikAV gene.

31. An isolated and purified DNA molecule comprising a DNA segment comprising a pikA promoter.

32. An expression cassette comprising a pikA promoter operably linked to a DNA molecule comprising a DNA segment comprising an open reading frame or a portion thereof.

33. The expression cassette of claim 32 wherein the DNA segment encodes the thioesterase domain of pikAIV.

34. The expression cassette of claim 32 wherein the DNA segment encodes the thioesterase II domain of pikAV.

35. The expression cassette of claim 33 further comprising an acyl carrier protein domain.

36. The expression cassette of claim 35 further comprising a thioesterase II domain.

37. The expression cassette of claim 35 fuirther comprising an acyl transferase domain.

38. The expression cassette of claim 37 further comprising a β-ketoacyl-acyl carrier protein synthase domain.

39. The expression cassette of any one of claims 32 to 38 wherein the DNA molecule comprises a second DNA segment comprising the leader sequence of pikAI operably linked to the first DNA segment.

40. A host cell transformed with a plasmid comprising the expression cassette of any one of claims 32 to 39.

41. The host cell of claim 40 which lacks the thioesterase domain of pikAIV gene cluster and the thioesterase II domain of pikAV gene.

42. A method to alter polyketide chain length, comprising: expressing in a host cell an expression cassette comprising at least a portion of a DNA segment that encodes a module that catalyzes the final condensation of a polyketide so as to yield a polyketide product which is of a different length relative to a polyketide produced by a host cell which does not express the module, wherein the DNA segment that encodes an intact module encodes two different polypeptides, one of which has a lower molecular weight than the other polypeptide.

43. The method of claim 42 wherein the intact module ispikA module 6.

44. The method of claim 42 wherein the expression cassette is present on a plasmid.

45. The method of claim 42 wherein the host cell is a polyketide-producing host cell.

46. A product produced by the method of claim 42 which is not produced by a host cell which does not express the module.

47. A method to prepare a polyketide product, comprising: expressing in a host cell an expression cassette comprising a promoter operably linked to a DNA segment comprising a portion of a first polyketide synthase gene so as to yield the product, wherein the expression cassette is present on a plasmid, wherein the chromosome of the host cell comprises at least a portion of a second polyketide synthase gene, and wherein both portions are operably linked to the native polyketide promoter of one of the polyketide genes.

48. The method of claim 47 wherein the portions are from the same polyketide gene and wherein the portion on the host cell chromosome is different than the portion that is on the plasmid.

49. The method of claim 48 wherein the portions together comprise the entire gene.

50. The method of claim 49 wherein the gene is the pikA gene cluster.

51. A host cell, the genome of which comprises at least a portion of a first polyketide synthase gene, comprising: a plasmid comprising a promoter operably linked to a DNA molecule comprising a DNA segment encoding a portion of a second polyketide synthase gene, wherein both portions are operably linked to the native promoter of one of the genes, and wherein the expression of both portions yields a polyketide.

52. The host cell of claim 51 wherein the portions are from the same polyketide gene and wherein the portion on the host cell genome is different than the portion that is on the plasmid.

53. The host cell of claim 52 wherein the portions together comprise the entire gene.

54. The host cell of claim 53 wherein the gene is the pikA gene cluster.

55. A polyketide produced by the host cell of claim 51.

56. Use of a product of claim 9, 21 or 46 for the manufacture of a medicament for the treatment of a pathological condition or symptom in a mammal.

57. The host cell of claim 7 wherein the nucleic acid sequence encoding DesR is disrupted.

58. A product produced by the host cell of claim 57.

59. The host cell of claim 7 wherein the nucleic acid sequence encoding DesI is disrupted.

60. A product produced by the host cell of claim 59.


SEQUENCE LISTING (53 sequences)

SEQ ID NO: 1 - DNA, 15872 bp, Streptomyces venezuelae
ttaattaaggaggaccatcatgaacgaggccatcgccgtcgtcggcatgtcctgccgcctgccgaaggcctcgaacccggccgccttctgggagctgctgcggaacggggagagcgccgtcaccgacgtgccctccggccggtggacgtcggtgctcgggggagcggacgccgaggagccggcggagtccggtgtccgccggggcggcttcctcgactccctcgacctcttcgacgcggccttcttcggaatctcgccccgtgaggccgccgccatggacccgcagcagcgactggtcctcgaactcgcctgggaggcgctggaggacgccggaatcgtccccggcaccctcgccggaagccgcaccgccgtcttcgtcggcaccctgcgggacgactacacgagcctcctctaccagcacggcgagcaggccatcacccagcacaccatggcgggcgtgaaccggggcgtcatcgccaaccgcgtctcgtaccacctcggcctgcagggcccgagcctcaccgtcgacgccgcgcagtcgtcctcgctcgtcgccgtgcacctggcctgcgagtccctgcgcgccggggagtccacgacggcgctcgtcgccggcgtgaacctcaacatcctcgcggagagcgccgtgacggaggagcgcttcggtggactctccccggacggcaccgcctacaccttcgacgcgcgggccaacggattcgtccggggcgagggcggcggagtcgtcgtactcaagccgctctcccgcgccctcgccgacggcgaccgtgtccacggcgtcatccgcgccagcgccgtcaacaacgacggagccaccccgggtctcaccgtgcccagcagggccgcccaggagaaggtgctgcgcgaggcgtaccggaaggcggccctggacccgtccgccgtccagtacgtcgaactccacggcaccggaacccccgtcggcgaccccatcgaggccgccgcgctcggcgccgtcctcggctcggcgcgccccgcggacgaacccctgctcgtcggctcggccaagacgaacgtcgggcacctcgaaggcgccgccggcatcgtcggcctcatcaagacgctcctcgcgctcggccggcgccggatcccggcgagcctcaacttccgtacgccccacccggacatcccgctcgacaccctcgggctcgacgtgcccgacggcctgcgggagtggccgcacccggaccgcgaactcctcgccggcgtcagctcgttcggcatgggcggcaccaacgcccacgtcgtcctcagcgaaggccccgcccagggcggcgagcagcccggcatcgatgaggagacccccgtcgacagcggggccgcactgcccttcgtcgtcaccggccgcggcggcgaggccctgcgcgcccaggcccggcgcctgcacgaggccgtcgaagcggacccggagctcgcgcccgccgcactcgcccggtcgctggtcaccacccgtacggtcttcacgcaccggtcggtcgtcctcgccccggaccgcgcccgcctcctcgacggcctcggcgccctcgccgccgggacgcccgcgcccggcgtggtcaccggcacccccgcccccgggcgcctcgccgtcctgttcagcggccagggtgcccaacgtacgggcatgggcatggagttgtacgccgcccaccccgccttcgcgacggccttcgacgccgtcgccgccgaactggaccccctcctcgaccggcccctcgccgaactcgtcgcggcgggcgacaccctcgaccgcaccgtccacacacagcccgcgctcttcgccgtggaggtcgccctccaccgcctcgtcgagtcctggggcgtcacgcccgacctgctcgccggccactccgtcggcgagatcagcgccgcccacgtcgccggggtcctgtcgctgcgcgacgccgcccgcctcgtcgcggcgcgcggccgcctcatgcaggcgctccccgagggcggcgcgatggtcgcggtcgaggcgagcgaggaggaagtgcttccgcacctcgcgggacgcgagcgggagctctccctcgcggccgtgaacggcccccgcgcggtcgtcctcgcgggcgccgagcgcgccgtcctcgacgtcgccgagctgctgcgcgaacagggccgccggacgaagcggctcagcgtctcgcacgccttccactcgccgctcatggagccgatgctcgacgacttccgccgggtcgtcgaagagctggacttccaggagccccgcgtcgacgtcgtgtccacggtgacgggcctgcctgtcacagcgggccaatggaccgatcccgagtactgggtggaccaggtccgcaggcccgtacgcttcctcgacgccgtacgcaccctggaggaatcgggcgccgacaccttcctggagctcggtcccgacggggtctgctccgcgatggcggcggactccgtacgcgaccaggaggccgccacggcggtctccgccctgcgcaagggccgcccggagccccagtcgctgctcgccgcactcaccaccgtcttcgtccggggccacgacgtcgactggaccgccgcgcacgggagcaccggcacggtcagggtgcccctgccgacctacgccttccagcgcgaacgccactggttcgacggcgccgcgcgaacggcggcgccgctcacggcgggccgatcgggcaccggtgcgggcaccggcccggccgcgggtgtgacgtcgggcgagggcgagggcgagggcgagggcgcgggtgcgggtggcggtgatcggccggctcgccacgagacgaccgagcgcgtgcgcgcacacgtcgccgccgtcctcgagtacgacgacccgacccgcgtcgaactcggcctcaccttcaaggagctgggcttcgactccctcatgtccgtcgagctgcggaacgcgctcgtcgacgacacgggactgcgcctgcccagcggactgctcttcgaccacccgacgccgcgcgccctcgccgcccacctgggcgacctgctcaccggcggcagcggcgagaccggatcggccgacgggataccgcccgcgaccccggcggacaccaccgccgagcccatcgcgatcatcggcatggcctgccgctaccccggcggcgtcacctcccccgaggacctgtggcggctcgtcgccgaggggcgcgacgccgtctcggggctgcccaccgaccgcggctgggacgaggacctcttcgacgccgaccccgaccgcagcggcaagagctcggtccgcgagggcggattcctgcacgacgccgccctgttcgacgccggcttcttcgggatatcgccccgcgaggccctcggcatggacccgcagcagcggctgctcctggagacggcatgggaggccgtggagcgcgcagggctcgaccccgaaggcctcaagggcagccggacggccgtcttcgtcggcgccaccgccctggactacggcccgcgcatgcacgacggcgccgagggcgtcgagggccacctcctgaccgggaccacgcccagcgtgatgtcgggccgcatcgcctaccagctcggcctcaccggtcctgcggtcaccgtcgacacggcctgctcgtcctcgctcgtcgcgctgcacctggccgtccgttcgctgcggcagggcgagtcgagcctcgcgctcgccggcggagcgaccgtcatgtcgacaccgggcatgttcgtcgagttctcgcggcagcgcggcctcgccgccgacggccgctccaaggccttctccgactccgccgacggcacctcctgggccgagggcgtcggcctcctcgtcgtcgagcggctctcggacgccgagcgcaacggccaccccgtgctcgccgtgatccggggcagcgcggtcaaccaggacggcgcctccaacgggctcaccgcccccaacggcccgtcccagcagcgcgtcatccgacaggccctggccgacgccgggctcaccccggccgacgtcgacgccgtcgaggcgcacggtacgggtacccggctcggcgaccccatcgaggccgaggcgatcctcggcacctacggccgggaccggggcgagggcgctccgctccagctcggctcgctgaagtcgaacatcggccacgcgcaggccgccgcgggcgtgggcgggctcatcaagatggtcctcgcgatgcgccacggcgtcctgcccaggacgctccacgtggaccggcccaccacccgcgtcgactgggaggccggcggcgtcgagctcctcaccgaggagcgggagtggccggagacgggccgcccgcgccgcgcggcgatctcctccttcggcatcagcggcaccaacgcccacatcgtggtcgaacaggccccggaagccggggaggcggcggtcaccaccaccgccccggaagcaggggaagccggggaagcggcggacaccaccgccaccacgacgccggccgcggtcggcgtccccgaacccgtacgcgcccccgtcgtggtctccgcgcgggacgccgccgccctgcgcgcccaggccgttcggctgcggaccttcctcgacggccgaccggacgtcaccgtcgccgacctcggacgctcgctggccgcccgtaccgccttcgagcacaaggccgccctcaccaccgccaccagggacgagctgctcgccgggctcgacgccctcggccgcggggagcaagccacgggcctggtcaccggcgaaccggccagggccggacgcacggccttcctgttcaccggccagggagcgcagcgcgtcgccatgggcgaggaactgcgcgccgcgcaccccgtgttcgccgccgccctcgacaccgtgtacgcggccctcgaccgtcacctcgaccggccgctgcgggagatcgtcgccgccggggaggagctggacctcaccgcgtacacccagcccgccctcttcgccttcgaggtggcgctgttccgcctcctcgaacaccacggcctcgtccccgacctgctcaccggccactccgtcggcgagatcgccgccgcgcacgtcgccggtgtcctctccctcgacgacgccgcacgtctcgtcaccgcccgcggccggctcatgcagtcggcccgcgagggcggcgcgatgatcgccgtgcaggcgggcgaggccgaggtcgtcgagtccctgaagggctacgagggcagggtcgccgtcgccgccgtcaacggacccaccgccgtggtcgtctccggcgacgcggacgccgccgaggagatccgcgccgtatgggcgggacgcggccggcgcacccgcaggctgcgcgtcagccacgccttccactccccgcacatggacgacgtcctcgacgagttcctccgggtcgccgagggcctgaccttcgaggagccgcggatccccgtcgtctccacggtcaccggcgcgctcgtcacgtccggcgagctcacctcgcccgcgtactgggtcgaccagatccggcggcccgtgcgcttcctggacgccgtccgcaccctggccgcccaggacgcgaccgtcctcgtcgagatcggccccgacgccgtcctcacggcactcgccgaggaggctctcgcgcccggcacggacgccccggacgcccgggacgtcacggtcgtcccgctgctgcgcgcggggcgccccgagcccgagaccctcgccgccggtctcgcgaccgcccatgtccacggcgcacccttggaccgggcgtcgttcttcccggacgggcgccgcacggacctgcccacgtacgccttccggcgcgagcactactggctgacgcccgaggcccgtacggacgcccgcgcactcggcttcgacccggcgcggcacccgctgctgacgaccacggtcgaggtcgccggcggcgacggcgtcctgctgaccggccgtctctccctgaccgaccagccctggctggccgaccacatggtcaacggcgccgtcctgttgccggccaccgccttcctggagctcgccctcgcggcgggcgaccacgtcggggcggtccgggtggaggaactcaccctcgaagcgccgctcgtcctgcccgagcggggcgccgtccgcatccaggtcggcgtgagcggcgacggcgagtcgccggccgggcgcaccttcggtgtgtacagcacccccgactccggcgacaccggtgacgacgcgccccgggagtggacccgccatgtctccggcgtactcggcgaaggggacccggccacggagtcggaccaccccggcaccgacggggacggttcagcggcctggccgcctgcggcggcgaccgccacacccctcgacggcgtctacgaccggctcgcggagctcggctacggatacggtccggccttccagggcctgacggggctgtggcgcgacggcgccgacacgctcgccgagatccggctgcccgcggcgcagcacgagagcgcggggctcttcggcgtacacccggcgctgctcgacgcggcgctccacccgatcgtcctggagggcaactcagctgccggtgcctgtgacgccgataccgacgcgaccgaccggatccggctgccgttcgcgtgggcgggggtgaccctccacgccgaaggggccaccgcgctccgcgtacggatcacacccaccggcccggacacggtcacgctccgcctcaccgacaccaccggtgcgcccgtggccaccgtggagtccctgaccctgcgcgcggtggcgaaggaccggctgggcaccaccgccgggcgcgtcgacgacgccctgttcacggtcgtgtggacggagaccggcacaccggaacccgcagggcgcggagccgtggaggtcgaggaactcgtcgacctcgccggcctcggcgacctcgtggagctcggcgccgcggacgtcgtcctccgggccgaccgctggacgctcgacggggacccgtccgccgccgcgcgcacagccgtccggcgcaccctcgccatcgtccaggagttcctgtccgagccgcgcttcgacggctcgcgactggtgtgcgtcaccaggggcgcggtcgccgcactccccggcgaggacgtcacctccctcgccaccggccccctctggggcctcgtccgctccgcccagtccgagaacccgggacgcctgttcctcctggacctgggtgaaggcgaaggcgagcgcgacggagccgaggagctgatccgcgcggccacggccggggacgagccgcagctcgcggcacgggacggccgactgctcgcgccgaggctggcccgtaccgccgccctttcgagtgaggacaccgccggcggcgccgaccgtttcggccccgacggcaccgtcctcgtcaccgggggcaccggaggcctcggagcgctcctcgcccgccacctcgtggagcgtcacggggtgcgccggctgctgctggtgagccgccgcggggccgacgcccccggcgcggccgacctgggcgaggacctcgcgggcctcggcgcggaggtggcgttcgccgccgccgacgccgccgaccgcgagagcctggcgcgggcgatcgccaccgtgcccgccgagcatccgctgacggccgtcgtgcacacggcgggagtcgtcgacgacgcgacggtggaggcgctcacaccggaacggctggacgcggtactgcgcccgaaggtcgacgccgcgtggaacctgcacgagctcaccaaggacctgcggctcgacgccttcgtcctcttctcctccgtctccggcatcgtcggcaccgccggccaggccaactacgcggcggccaacacgggcctcgacgccctcgccgcccaccgcgccgccacgggcctggccgccacgtcgctggcctggggcctctgggacggcacgcacggcatgggcggcacgctcggcgccgccgacctcgcccgctggagccgggccggaatcaccccgctcaccccgctgcagggcctcgcgctcttcgacgccgcggtcgccagggacgacgccctcctcgtacccgccgggctccgtcccaccgcccaccggggcacggacggacagcctcctgcgctgtggcgcggcctcgtccgggcgcgcccgcgccgtgccgcgcggacggccgccgaggcggcggacacgaccggcggctggctgagcgggctcgccgcacagtcccccgaggagcggcgcagcacagccgtcacgctcgtgacgggtgtcgtcgcggacgtcctcgggcacgccgactccgccgcggtcggggcggagcggtccttcaaggacctcggcttcgactccctggccggggtggagctccgcaaccggctgaacgccgccaccggcctgcggctccccgcgaccacggtcttcgaccatccctcgccggccgcgctcgcgtcccatctcctcgcccaggtgcccgggttgaaggaggggacggcggcgaccgcgaccgtcgtggccgagcggggcgcttccttcggtgaccgtgcgaccgacgacgatccgatcgcgatcgtgggcatggcatgccgctatccgggtggtgtgtcgtcgccggaggacctgtggcggctggtggccgaggggacggacgcgatcagcgagttccccgtcaaccgcggctgggacctggagagcctctacgacccggatcccgagtcgaagggcaccacgtactgccgggagggcgggttcctggaaggcgccggtgacttcgacgccgccttcttcggcatctcgccgcgcgaggccctggtgatggacccgcagcagcggctgctgctggaggtgtcctgggaggcgctggaacgcgcgggcatcgacccgtcctcgctgcgcggcagccgcggtggtgtctacgtgggcgccgcgcacggctcgtacgcctccgatccccggctggtgcccgagggctcggagggctatctgctgaccggcagcgccgacgcggtgatgtccggccgcatctcctacgcgctcggtctcgaaggaccgtccatgacggtggagacggcctgctcctcctcgctggtggcgctgcatctggcggtacgggcgctgcggcacggcgagtgcgggctcgcgctggcgggcggggtggcggtgatggccgatccggcggcgttcgtggagttctcccggcagaaggggctggccgccgacggccgctgcaaggcgttctcggccgccgccgacggcaccggctgggccgagggcgtcggcgtgctcgtcctggagcggctgtcggacgcgcgccgcgcggggcacacggtcctcggcctggtcaccggcaccgcggtcaaccaggacggtgcctccaacgggctgaccgcgcccaacggcccagcccagcaacgcgtcatcgccgaggcgctcgccgacgccgggctgtccccggaggacgtggacgcggtcgaggcgcacggcaccggcacccggctcggcgaccccatcgaggccggggcgctgctcgccgcctccggacggaaccgttccggcgaccacccgctgtggctcggctcgctgaagtccaacatcgggcatgcccaggccgccgccggtgtcggcggcgtcatcaagatgctccaggcgctgcggcacggcttgctgccccgcaccctccacgccgacgagccgaccccgcatgccgactggagctccggccgggtacggctgctcacctccgaggtgccgtggcagcggaccggccggccccggcggaccggggtgtccgccttcggcgtcggcggcaccaatgcccatgtcgtcctcgaagaggcacccgccccgcccgcgccggaaccggccggggaggcccccggcggctcccgcgccgcagaaggggcggaagggcccctggcctgggtggtctccggacgcgacgagccggccctgcggtcccaggcccggcggctccgcgaccacctctcccgcacccccggggcccgcccgcgtgacatcgccttctccctcgccgccacgcgcgcagcctttgaccaccgcgccgtgctgatcggctcggacggggccgaactcgccgccgccctggacgcgttggccgaaggacgcgacggtccggcggtggtgcgcggagtccgcgaccgggacggcaggatggccttcctcttcaccgggcagggcagccagcgcgccgggatggcccacgacctgcatgccgcccataccttcttcgcgtccgccctcgacgaggtgacggaccgtctcgacccgctgctcggccggccgctcggcgcgctgctggacgcccgacccggctcgcccgaagcggcactcctggaccggaccgagtacacccagccggcgctcttcgccgtcgaggtggcgctccaccggctgctggagcactgggggatgcgccccgacctgctgctggggcactcggtgggcgaactggcggccgcccacgtcgcgggtgtgctcgatctcgacgacgcctgcgcgctggtggccgcccgcggcaggctgatgcagcgcctgccgcccggcggcgcgatggtctccgtgcgggccggcgaggacgaggtccgcgcactgctggccggccgcgaggacgccgtctgcgtcgccgcggtgaacggcccccggtcggtggtgatctccggcgcggaggaagcggtggccgaggcggcggcgcagctcgccggacgaggccgccgcaccaggcggctccgcgtcgcgcacgccttccactcacccctgatggacggcatgctcgccggattccgggaggtcgccgccggcctgcgctaccgggaaccggagctgacggtcgtctccacggtcacggggcggcccgcccgccccggtgaactcaccggccccgactactgggtggcccaggtccgtgagcccgtgcgcttcgcggacgcggtccgcacggcacaccgcctcggagcccgcaccttcctggagaccggcccggacggcgtgctgtgcggcatggcagaggagtgcctggaggacgacaccgtggccctgctgccggcgatccacaagcccggcaccgcgccgcacggtccggcggctcccggcgcgctgcgggcggccgccgccgcgtacggccggggcgcccgggtggactgggccgggatgcacgccgacggccccgaggggccggcccgccgcgtcgaactgcccgtccacgccttccggcaccgccgctactggctcgccccgggccgcgcggcggacaccgacgactggatgtaccggatcggctgggaccggctgccggctgtgaccggcggggcccggaccgccggccgctggctggtgatccaccccgacagcccgcgctgccgggagctgtccggccacgccgaacgcgcgctgcgcgccgcgggcgcgagccccgtaccgctgcccgtggacgctccggccgccgaccgggcgtccttcgcggcactgctgcgctccgccaccggacctgacacacgaggtgacacagccgcgcccgtggccggtgtgctgtcgctgctgtccgaggaggatcggccccatcgccagcacgccccggtacccgccggggtcctggcgacgctgtccctgatgcaggctatggaggaggaggcggtggaggctcgcgtgtggtgcgtctcccgcgccgcggtcgccgccgccgaccgggaacggcccgtcggcgcgggcgccgccctgtgggggctggggcgggtggccgccctggaacgccccacccggtggggcggtctcgtggacctgcccgcctcgcccggtgcggcgcactgggcggccgccgtggaacggctcgccggtcccgaggaccagatcgccgtgcgcgcgtccggcagttggggccggcgcctcaccaggctgccgcgcgacggcggcggccggacggccgcacccgcgtaccggccgcgcggcacggtgctcgtcaccggtggcaccggcgcgctcggcgggcatctcgcccgctggctcgccgcggcgggcgccgaacacctggcgctcaccagccgccggggcccggacgcgcccggcgccgccggactcgaggccgaactcctcctcctgggcgccaaggtgacgttcgccgcctgcgacaccgccgaccgcgacggcctcgcccgggtcctgcgggcgataccggaggacaccccgctcaccgcggtgttccacgccgcgggcgtaccgcaggtcacgccgctgtcccgtacctcgcccgagcacttcgccgacgtgtacgcgggcaaggcggcgggcgccgcgcacctggacgaactgacccgcgaactcggcgccggactcgacgcgttcgtcctctactcctccggcgccggcgtctggggcagcgccggccagggtgcctacgccgccgccaacgccgccctggacgcgctcgcccggcgccgtgcggcggacggactccccgccacctccatcgcctggggcgtgtggggcggcggcggtatgggggccgacgaggcgggcgcggagtatctgggccggcgcggtatgcgccccatggcaccggtctccgcgctccgggcgatggccaccgccatcgcctccggggaaccctgccccaccgtcacccacaccgactgggagcgcttcggcgagggcttcaccgccttccggcccagccctctgatcgcggggctcggcacgccgggcggcggccgggcggcggagacccccgaggaggggaacgccaccgctgcggcggacctcaccgccctgccgcccgccgaactccgcaccgcgctgcgcgagctggtgcgagcccggaccgccgcggcgctcggcctcgacgacccggccgaggtcgccgagggcgaacggttccccgccatgggcttcgactccctggccaccgtacggctgcgccgcggactcgcctcggccacgggcctcgacctgccccccgatctgctcttcgaccgggacaccccggccgcgctcgccgcccacctggccgaactgctcgccaccgcacgggaccacggacccggcggccccgggaccggtgccgcgccggccgatgccggaagcggcctgccggccctctaccgggaggccgtccgcaccggccgggccgcggaaatggccgaactgctcgccgccgcttcccggttccgccccgccttcgggacggcggaccggcagccggtggccctcgtgccgctggccgacggcgcggaggacaccgggctcccgctgctcgtgggctgcgccgggacggcggtggcctccggcccggtggagttcaccgccttcgccggagcgctggcggacctcccggcggcggccccgatggccgcgctgccgcagcccggctttctgccgggagaacgagtcccggccaccccggaggcattgttcgaggcccaggcggaagcgctgctgcgctacgcggccggccggcccttcgtgctgctggggcactccgccggcgccaacatggcccacgccctgacccgtcatctggaggcgaacggtggcggccccgcagggctggtgctcatggacatctacacccccgccgaccccggcgcgatgggcgtctggcggaacgacatgttccagtgggtctggcggcgctcggacatccccccggacgaccaccgcctcacggccatgggcgcctaccaccggctgcttctcgactggtcgcccacccccgtccgcgcccccgtactgcatctgcgcgccgcggaacccatgggcgactggccacccggggacaccggctggcagtcccactgggacggcgcgcacaccaccgccggcatccccggaaaccacttcacgatgatgaccgaacacgcctccgccgccgcccggctcgtgcacggctggctcgcggaacggaccccgtccgggcagggcgggtcaccgtcccgcgcggcggggagagaggagaggccgtgaacacggcagccggcccgaccggcaccgccgccggcggcaccaccgccccggcggcggcacacgacctgtcccgcgccggacgcaggctccaactcacccgggccgcacagtggttcgccggcaaccagggagacccctacgggatgatcctgcgcgccggcaccgccgacccggcaccgtacgaggaagagatccccgggtaccgagctcgaattcttaattaaggaggtcgtagatgagtaacaagaacaacgatgagctgcagcggcaggcctcggaaaacaccctggggctgaacccggtcatcggtatccgccgcaaagacctgttgagctcggcacgcaccgtgctgcgccaggccgtgcgccaaccgctgcacagcgccaagcatgtggcccactttggcctggagctgaagaacgtgctgctgggcaagtccagccttgccccggaaagcgacgaccgtcgcttcaatgacccggcatggagcaacaacccactttaccgccgctacctgcaaacctatctggcctggcgcaaggagctgcaggactggatcggcaacagcgacctgtcgccccaggacatcagccgcggccagttcgtcatcaacctgatgaccgaagccatggctccgaccaacaccctgtccaacccggcagcagtcaaacgcttcttcgaaaccggcggcaagagcctgctcgatggcctgtccaacctggccaaggacctggtcaacaacggtggcatgcccagccaggtgaacatggacgccttcgaggtgggcaagaacctgggcaccagtgaaggcgccgtggtgtaccgcaacgatgtgctggagctgatccagtacaagcccatcaccgagcaggtgcatgcccgcccgctgctggtggtgccgccgcagatcaacaagttctacgtattcgacctgagcccggaaaagagcctggcacgctactgcctgcgctcgcagcagcagaccttcatcatcagctggcgcaacccgaccaaagcccagcgcgaatggggcctgtccacctacatcgacgcgctcaaggaggcggtcgacgcggtgctggcgattaccggcagcaaggacctgaacatgctcggtgcctgctccggcggcatcacctgcacggcattggtcggccactatgccgccctcggcgaaaacaaggtcaatgccctgaccctgctggtcagcgtgctggacaccaccatggacaaccaggtcgccctgttcgtcgacgagcagactttggaggccgccaagcgccactcctaccaggccggtgtgctcgaaggcagcgagatggccaaggtgttcgcctggatgcgccccaacgacctgatctggaactactgggtcaacaactacctgctcggcaacgagccgccggtgttcgacatcctgttctggaacaacgacaccacgcgcctgccggccgccttccacggcgacctgatcgaaatgttcaagagcaacccgctgacccgcccggacgccctggaggtttgcggcactccgatcgacctgaaacaggtcaaatgcgacatctacagccttgccggcaccaacgaccacatcaccccgtggcagtcatgctaccgctcggcgcacctgttcggcggcaagatcgagttcgtgctgtccaacagcggccacatccagagcatcctcaacccgccaggcaaccccaaggcgcgcttcatgaccggtgccgatcgcccgggtgacccggtggcctggcaggaaaacgccaccaagcatgccgactcctggtggctgcactggcaaagctggctgggcgagcgtgccggcgagctggaaaaggcgccgacccgcctgggcaaccgtgcctatgccgctggcgaggcatccccgggcacctacgttcacgagcgttgagctgcagcgccgtggccacctgcgggacgccacggtgttgaattc

SEQ ID NO: 2 - protein, 5215 aa, Streptomyces venezuelae
MNEAIAVVGMSCRLPKASNPAAFWELLRNGESAVTDVPSGRWTSVLGGADAEEPAESGVRRGGFLDSLDLFDAAFFGISPREAAAMDPQQRLVLELAWEALEDAGIVPGTLAGSRTAVFVGTLRDDYTSLLYQHGEQAITQHTMAGVNRGVIANRVSYHLGLQGPSLTVDAAQSSSLVAVHLACESLRAGESTTALVAGVNLNILAESAVTEERFGGLSPDGTAYTFDARANGFVRGEGGGVVVLKPLSRALADGDRVHGVIRASAVNNDGATPGLTVPSRAAQEKVLREAYRKAALDPSAVQYVELHGTGTPVGDPIEAAALGAVLGSARPADEPLLVGSAKTNVGHLEGAAGIVGLIKTLLALGRRRIPASLNFRTPHPDIPLDTLGLDVPDGLREWPHPDRELLAGVSSFGMGGTNAHVVLSEGPAQGGEQPGIDEETPVDSGAALPFVVTGRGGEALRAQARRLHEAVEADPELAPAALARSLVTTRTVFTHRSVVLAPDRARLLDGLGALAAGTPAPGVVTGTPAPGRLAVLFSGQGAQRTGMGMELYAAHPAFATAFDAVAAELDPLLDRPLAELVAAGDTLDRTVHTQPALFAVEVALHRLVESWGVTPDLLAGHSVGEISAAHVAGVLSLRDAARLVAARGRLMQALPEGGAMVAVEASEEEVLPHLAGRERELSLAAVNGPRAVVLAGAERAVLDVAELLREQGRRTKRLSVSHAFHSPLMEPMLDDFRRVVEELDFQEPRVDVVSTVTGLPVTAGQWTDPEYWVDQVRRPVRFLDAVRTLEESGADTFLELGPDGVCSAMAADSVRDQEAATAVSALRKGRPEPQSLLAALTTVFVRGHDVDWTAAHGSTGTVRVPLPTYAFQRERHWFDGAARTAAPLTAGRSGTGAGTGPAAGVTSGEGEGEGEGAGAGGGDRPARHETTERVRAHVAAVLEYDDPTRVELGLTFKELGFDSLMSVELRNALVDDTGLRLPSGLLFDHPTPRALAAHLGDLLTGGSGETGSADGIPPATPADTTAEPIAIIGMACRYPGGVTSPEDLWRLVAEGRDAVSGLPTDRGWDEDLFDADPDRSGKSSVREGGFLHDAALFDAGFFGISPREALGMDPQQRLLLETAWEAVERAGLDPEGLKGSRTAVFVGATALDYGPRMHDGAEGVEGHLLTGTTPSVMSGRIAYQLGLTGPAVTVDTACSSSLVALHLAVRSLRQGESSLALAGGATVMSTPGMFVEFSRQRGLAADGRSKAFSDSADGTSWAEGVGLLVVERLSDAERNGHPVLAVIRGSAVNQDGASNGLTAPNGPSQQRVIRQALADAGLTPADVDAVEAHGTGTRLGDPIEAEAILGTYGRDRGEGAPLQLGSLKSNIGHAQAAAGVGGLIKMVLAMRHGVLPRTLHVDRPTTRVDWEAGGVELLTEEREWPETGRPRRAAISSFGISGTNAHIVVEQAPEAGEAAVTTTAPEAGEAGEAADTTATTTPAAVGVPEPVRAPVVVSARDAAALRAQAVRLRTFLDGRPDVTVADLGRSLAARTAFEHKAALTTATRDELLAGLDALGRGEQATGLVTGEPARAGRTAFLFTGQGAQRVAMGEELRAAHPVFAAALDTVYAALDRHLDRPLREIVAAGEELDLTAYTQPALFAFEVALFRLLEHHGLVPDLLTGHSVGEIAAAHVAGVLSLDDAARLVTARGRLMQSAREGGAMIAVQAGEAEVVESLKGYEGRVAVAAVNGPTAVVVSGDADAAEEIRAVWAGRGRRTRRLRVSHAFHSPHMDDVLDEFLRVAEGLTFEEPRIPVVSTVTGALVTSGELTSPAYWVDQIRRPVRFLDAVRTLAAQDATVLVEIGPDAVLTALAEEALAPGTDAPDARDVTVVPLLRAGRPEPETLAAGLATAHVHGAPLDRASFFPDGRRTDLPTYAFRREHYWLTPEARTDARALGFDPARHPLLTTTVEVAGGDGVLLTGRLSLTDQPWLADHMVNGAVLLPATAFLELALAAGDHVGAVRVEELTLEAPLVLPERGAVRIQVGVSGDGESPAGRTFGVYSTPDSGDTGDDAPREWTRHVSGVLGEGDPATESDHPGTDGDGSAAWPPAAATATPLDGVYDRLAELGYGYGPAFQGLTGLWRDGADTLAEIRLPAAQHESAGLFGVHPALLDAALHPIVLEGNSAAGACDADTDATDRIRLPFAWAGVTLHAEGATALRVRITPTGPDTVTLRLTDTTGAPVATVESLTLRAVAKDRLGTTAGRVDDALFTVVWTETGTPEPAGRGAVEVEELVDLAGLGDLVELGAADVVLRADRWTLDGDPSAAARTAVRRTLAIVQEFLSEPRFDGSRLVCVTRGAVAALPGEDVTSLATGPLWGLVRSAQSENPGRLFLLDLGEGEGERDGAEELIRAATAGDEPQLAARDGRLLAPRLARTAALSSEDTAGGADRFGPDGTVLVTGGTGGLGALLARHLVERHGVRRLLLVSRRGADAPGAADLGEDLAGLGAEVAFAAADAADRESLARAIATVPAEHPLTAVVHTAGVVDDATVEALTPERLDAVLRPKVDAAWNLHELTKDLRLDAFVLFSSVSGIVGTAGQANYAAANTGLDALAAHRAATGLAATSLAWGLWDGTHGMGGTLGAADLARWSRAGITPLTPLQGLALFDAAVARDDALLVPAGLRPTAHRGTDGQPPALWRGLVRARPRRAARTAAEAADTTGGWLSGLAAQSPEERRSTAVTLVTGVVADVLGHADSAAVGAERSFKDLGFDSLAGVELRNRLNAATGLRLPATTVFDHPSPAALASHLLAQVPGLKEGTAATATVVAERGASFGDRATDDDPIAIVGMACRYPGGVSSPEDLWRLVAEGTDAISEFPVNRGWDLESLYDPDPESKGTTYCREGGFLEGAGDFDAAFFGISPREALVMDPQQRLLLEVSWEALERAGIDPSSLRGSRGGVYVGAAHGSYASDPRLVPEGSEGYLLTGSADAVMSGRISYALGLEGPSMTVETACSSSLVALHLAVRALRHGECGLALAGGVAVMADPAAFVEFSRQKGLAADGRCKAFSAAADGTGWAEGVGVLVLERLSDARRAGHTVLGLVTGTAVNQDGASNGLTAPNGPAQQRVIAEALADAGLSPEDVDAVEAHGTGTRLGDPIEAGALLAASGRNRSGDHPLWLGSLKSNIGHAQAAAGVGGVIKMLQALRHGLLPRTLHADEPTPHADWSSGRVRLLTSEVPWQRTGRPRRTGVSAFGVGGTNAHVVLEEAPAPPAPEPAGEAPGGSRAAEGAEGPLAWVVSGRDEPALRSQARRLRDHLSRTPGARPRDIAFSLAATRAAFDHRAVLIGSDGAELAAALDALAEGRDGPAVVRGVRDRDGRMAFLFTGQGSQRAGMAHDLHAAHTFFASALDEVTDRLDPLLGRPLGALLDARPGSPEAALLDRTEYTQPALFAVEVALHRLLEHWGMRPDLLLGHSVGELAAAHVAGVLDLDDACALVAARGRLMQRLPPGGAMVSVRAGEDEVRALLAGREDAVCVAAVNGPRSVVISGAEEAVAEAAAQLAGRGRRTRRLRVAHAFHSPLMDGMLAGFREVAAGLRYREPELTVVSTVTGRPARPGELTGPDYWVAQVREPVRFADAVRTAHRLGARTFLETGPDGVLCGMAEECLEDDTVALLPAIHKPGTAPHGPAAPGALRAAAAAYGRGARVDWAGMHADGPEGPARRVELPVHAFRHRRYWLAPGRAADTDDWMYRIGWDRLPAVTGGARTAGRWLVIHPDSPRCRELSGHAERALRAAGASPVPLPVDAPAADRASFAALLRSATGPDTRGDTAAPVAGVLSLLSEEDRPHRQHAPVPAGVLATLSLMQAMEEEAVEARVWCVSRAAVAAADRERPVGAGAALWGLGRVAALERPTRWGGLVDLPASPGAAHWAAAVERLAGPEDQIAVRASGSWGRRLTRLPRDGGGRTAAPAYRPRGTVLVTGGTGALGGHLARWLAAAGAEHLALTSRRGPDAPGAAGLEAELLLLGAKVTFAACDTADRDGLARVLRAIPEDTPLTAVFHAAGVPQVTPLSRTSPEHFADVYAGKAAGAAHLDELTRELGAGLDAFVLYSSGAGVWGSAGQGAYAAANAALDALARRRAADGLPATSIAWGVWGGGGMGADEAGAEYLGRRGMRPMAPVSALRAMATAIASGEPCPTVTHTDWERFGEGFTAFRPSPLIAGLGTPGGGRAAETPEEGNATAAADLTALPPAELRTALRELVRARTAAALGLDDPAEVAEGERFPAMGFDSLATVRLRRGLASATGLDLPPDLLFDRDTPAALAAHLAELLATARDHGPGGPGTGAAPADAGSGLPALYREAVRTGRAAEMAELLAAASRFRPAFGTADRQPVALVPLADGAEDTGLPLLVGCAGTAVASGPVEFTAFAGALADLPAAAPMAALPQPGFLPGERVPATPEALFEAQAEALLRYAAGRPFVLLGHSAGANMAHALTRHLEANGGGPAGLVLMDIYTPADPGAMGVWRNDMFQWVWRRSDIPPDDHRLTAMGAYHRLLLDWSPTPVRAPVLHLRAAEPMGDWPPGDTGWQSHWDGAHTTAGIPGNHFTMMTEHASAAARLVHGWLAERTPSGQGGSPSRAAGREERPMILRAGTADPAPYEEEIPGYRARILNMSNKNNDELQRQASENTLGLNPVIGIRRKDLLSSARTVLRQAVRQPLHSAKHVAHFGLELKNVLLGKSSLAPESDDRRFNDPAWSNNPLYRRYLQTYLAWRKELQDWIGNSDLSPQDISRGQFVINLMTEAMAPTNTLSNPAAVKRFFETGGKSLLDGLSNLAKDLVNNGGMPSQVNMDAFEVGKNLGTSEGAVVYRNDVLELIQYKPITEQVHARPLLVVPPQINKFYVFDLSPEKSLARYCLRSQQQTFIISWRNPTKAQREWGLSTYIDALKEAVDAVLAITGSKDLNMLGACSGGITCTALVGHYAALGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTPIDLKQVKCDIYSLAGTNDHITPWQSCYRSAHLFGGKIEFVLSNSGHIQSILNPPGNPKARFMTGADRPGDPVAWQENATKHADSWWLHWQSWLGERAGELEKAPTRLGNRAYAAGEASPGTYVHER

SEQ ID NO: 3 - DNA, 12441 bp, Streptomyces venezuelae
agatctgcaacgacatctccgaccacctgctcgtcacccgcggcgcgcccgatgcccgcgtcgtgcagcccccgaccagccttatcgaaggagcggcgaagagatggcagaacccacggtgaccgacgacctgacgggggccctcacgcagcccccgctgggccgcaccgtccgcgcggtggccgaccgtgaactcggcacccacctcctggagacccgcggcatccactggatccacgccgcgaacggcgacccgtacgccaccgtgctgcgcggccaggcggacgacccgtatcccgcgtacgagcgggtgcgtgcccgcggcgcgctctccttcagcccgacgggcagctgggtcaccgccgatcacgccctggcggcgagcatcctctgctcgacggacttcggggtctccggcgccgacggcgtcccggtgccgcagcaggtcctctcgtacggggagggctgtccgctggagcgcgagcaggtgctgccggcggccggtgacgtgccggagggcgggcagcgtgccgtggtcgaggggatccaccgggagacgctggagggtctcgcgccggacccgtcggcgtcgtacgccttcgagctgctgggcggtttcgtccgcccggcggtgacggccgctgccgccgccgtgctgggtgttcccgcggaccggcgcgcggacttcgcggatctgctggagcggctccggccgctgtccgacagcctgctggccccgcagtccctgcggacggtacgggcggcggacggcgcgctggccgagctcacggcgctgctcgccgattcggacgactcccccggggccctgctgtcggcgctcggggtcaccgcagccgtccagctcaccgggaacgcggtgctcgcgctcctcgcgcatcccgagcagtggcgggagctgtgcgaccggcccgggctcgcggcggccgcggtggaggagaccctccgctacgacccgccggtgcagctcgacgcccgggtggtccgcggggagacggagctggcgggccggcggctgccggccggggcgcatgtcgtcgtcctgaccgccgcgaccggccgggacccggaggtcttcacggacccggagcgcttcgacctcgcgcgccccgacgccgccgcgcacctcgcgctgcaccccgccggtccgtacggcccggtggcgtccctggtccggcttcaggcggaggtcgcgctgcggaccctggccgggcgtttccccgggctgcggcaggcgggggacgtgctccgcccccgccgcgcgcctgtcggccgcgggccgctgagcgtcccggtcagcagctcctgagacaccggggccccggtccgcccggccccccttcggacggaccggacggctcggaccacggggacggctcagaccgtcccgtgtgtccccgtccggctcccgtccgccccatcccgcccctccaccggcaaggaaggacacgacgccatgcgcgtcctgctgacctcgttcgcacatcacacgcactactacggcctggtgcccctggcctgggcgctgctcgccgccgggcacgaggtgcgggtcgccagccagcccgcgctcacggacaccatcaccgggtccgggctcgccgcggtgccggtcggcaccgaccacctcatccacgagtaccgggtgcggatggcgggcgagccgcgcccgaaccatccggcgatcgccttcgacgaggcccgtcccgagccgctggactgggaccacgccctcggcatcgaggcgatcctcgccccgtacttccatctgctcgccaacaacgactcgatggtcgacgacctcgtcgacttcgcccggtcctggcagccggacctggtgctgtgggagccgacgacctacgcgggcgccgtcgccgcccaggtcaccggtgccgcgcacgcccgggtcctgtgggggcccgacgtgatgggcagcgcccgccgcaagttcgtcgcgctgcgggaccggcagccgcccgagcaccgcgaggaccccaccgcggagtggctgacgtggacgctcgaccggtacggcgcctccttcgaagaggagctgctcaccggccagttcacgatcgacccgaccccgccgagcctgcgcctcgacacgggcctgccgaccgtcgggatgcgttatgttccgtacaacggcacgtcggtcgtgccggactggctgagtgagccgcccgcgcggccccgggtctgcctgaccctcggcgtctccgcgcgtgaggtcctcggcggcgacggcgtctcgcagggcgacatcctggaggcgctcgccgacctcgacatcgagctcgtcgccacgctcgacgcgagtcagcgcgccgagatccgcaactacccgaagcacacccggttcacggacttcgtgccgatgcacgcgctcctgccgagctgctcggcgatcatccaccacggcggggcgggcacctacgcgaccgccgtgatcaacgcggtgccgcaggtcatgctcgccgagctgtgggacgcgccggtcaaggcgcgggccgtcgccgagcagggggcggggttcttcctgccgccggccgagctcacgccgcaggccgtgcgggacgccgtcgtccgcatcctcgacgacccctcggtcgccaccgccgcgcaccggctgcgcgaggagaccttcggcgaccccaccccggccgggatcgtccccgagctggagcggctcgccgcgcagcaccgccgcccgccggccgacgcccggcactgagccgcacccctcgccccaggcctcacccctgtatctgcgccgggggacgcccccggcccaccctccgaaagaccgaaagcaggagcaccgtgtacgaagtcgaccacgccgacgtctacgacctcttctacctgggtcgcggcaaggactacgccgccgaggcctccgacatcgccgacctggtgcgctcccgtacccccgaggcctcctcgctcctggacgtggcctgcggtacgggcacgcatctggagcacttcaccaaggagttcggcgacaccgccggcctggagctgtccgaggacatgctcacccacgcccgcaagcggctgcccgacgccacgctccaccagggcgacatgcgggacttccggctcggccggaagttctccgccgtggtcagcatgttcagctccgtcggctacctgaagacgaccgaggaactcggcgcggccgtcgcctcgttcgcggagcacctggagcccggtggcgtcgtcgtcgtcgagccgtggtggttcccggagaccttcgccgacggctgggtcagcgccgacgtcgtccgccgtgacgggcgcaccgtggcccgtgtctcgcactcggtgcgggaggggaacgcgacgcgcatggaggtccacttcaccgtggccgacccgggcaagggcgtgcggcacttctccgacgtccatctcatcaccctgttccaccaggccgagtacgaggccgcgttcacggccgccgggctgcgcgtcgagtacctggagggcggcccgtcgggccgtggcctcttcgtcggcgtccccgcctgagcaccgcccaagaccccccggggcgggacgtcccgggtgcaccaagcaaagagagagaaacgaaccgtgacaggtaagacccgaataccgcgtgtccgccgcggccgcaccacgcccagggccttcaccctggccgtcgtcggcaccctgctggcgggcaccaccgtggcggccgccgctcccggcgccgccgacacggccaatgttcagtacacgagccgggcggcggagctcgtcgcccagatgacgctcgacgagaagatcagcttcgtccactgggcgctggaccccgaccggcagaacgtcggctaccttcccggcgtgccgcgtctgggcatcccggagctgcgtgccgccgacggcccgaacggcatccgcctggtggggcagaccgccaccgcgctgcccgcgccggtcgccctggccagcaccttcgacgacaccatggccgacagctacggcaaggtcatgggccgcgacggtcgcgcgctcaaccaggacatggtcctgggcccgatgatgaacaacatccgggtgccgcacggcggccggaactacgagaccttcagcgaggaccccctggtctcctcgcgcaccgcggtcgcccagatcaagggcatccagggtgcgggtctgatgaccacggccaagcacttcgcggccaacaaccaggagaacaaccgcttctccgtgaacgccaatgtcgacgagcagacgctccgcgagatcgagttcccggcgttcgaggcgtcctccaaggccggcgcggcctccttcatgtgtgcctacaacggcctcaacgggaagccgtcctgcggcaacgacgagctcctcaacaacgtgctgcgcacgcagtggggcttccagggctgggtgatgtccgactggctcgccaccccgggcaccgacgccatcaccaagggcctcgaccaggagatgggcgtcgagctccccggcgacgtcccgaagggcgagccctcgccgccggccaagttcttcggcgaggcgctgaagacggccgtcctgaacggcacggtccccgaggcggccgtgacgcggtcggcggagcggatcgtcggccagatggagaagttcggtctgctcctcgccactccggcgccgcggcccgagcgcgacaaggcgggtgcccaggcggtgtcccgcaaggtcgccgagaacggcgcggtgctcctgcgcaacgagggccaggccctgccgctcgccggtgacgccggcaagagcatcgcggtcatcggcccgacggccgtcgaccccaaggtcaccggcctgggcagcgcccacgtcgtcccggactcggcggcggcgccactcgacaccatcaaggcccgcgcgggtgcgggtgcgacggtgacgtacgagacgggtgaggagaccttcgggacgcagatcccggcggggaacctcagcccggcgttcaaccagggccaccagctcgagccgggcaaggcgggggcgctgtacgacggcacgctgaccgtgcccgccgacggcgagtaccgcatcgcggtccgtgccaccggtggttacgccacggtgcagctcggcagccacaccatcgaggccggtcaggtctacggcaaggtgagcagcccgctcctcaagctgaccaagggcacgcacaagctcacgatctcgggcttcgcgatgagtgccaccccgctctccctggagctgggctgggtgacgccggcggcggccgacgcgacgatcgcgaaggccgtggagtcggcgcggaaggcccgtacggcggtcgtcttcgcctacgacgacggcaccgagggcgtcgaccgtccgaacctgtcgctgccgggtacgcaggacaagctgatctcggctgtcgcggacgccaacccgaacacgatcgtggtcctcaacaccggttcgtcggtgctgatgccgtggctgtccaagacccgcgcggtcctggacatgtggtacccgggccaggcgggcgccgaggccaccgccgcgctgctctacggtgacgtcaacccgagcggcaagctcacgcagagcttcccggccgccgagaaccagcacgcggtcgccggcgacccgacaagctacccgggcgtcgacaaccagcagacgtaccgcgagggcatccacgtcgggtaccgctggttcgacaaggagaacgtcaagccgctgttcccgttcgggcacggcctgtcgtacacctcgttcacgcagagcgccccgaccgtcgtgcgtacgtccacgggtggtctgaaggtcacggtcacggtccgcaacagcgggaagcgcgccggccaggaggtcgtccaggcgtacctcggtgccagcccgaacgtgacggctccgcaggcgaagaagaagctcgtgggctacacgaaggtctcgctcgccgcgggcgaggcgaagacggtgacggtgaacgtcgaccgccgtcagctgcagaccggttcgtcctccgccgacctgcggggcagcgccacggtcaacgtctggtgacgtgacgccgtgaaagcggcggtgcccgccacccgggagggtggcgggcaccgctttttcggcctgctgggtctaccggaccacctgactaggcctggtcgacccgctcggcccattcgcgcacggcgtcgatcacccgcagcgcctgcgggcgctccaggtgcgggccgatcggcaggctgaggacctgccgcgcgaagctctcggcccgcgggagcgagccttccggcggtgcctcgcccgcgtaggcgggcgagaggtgcacgggtaccgggtagtgcgtgagggtgtcgatgccgcgggcgtcgaggtggctgcgcagctcgtcgcggcgctcggtgcgcacggtgaagaggtgccagaccgggtcggtgtcgggcgcggtcaccggcaggccgatgccgggcagtccggcgagcccggagaggtactccgcggccagcgccgacctgcggccgttccagctgtccaggtgggcgagccggatccgcagcacggcggcctgcatctcgtccaggcgggagttggtgcccttcgtctcgtggctgtacttctgccgcgagccgtagttgcggagcatccggagccgttcggcgagctcggggtcgccggtgacgacggcgccgccgtcgccgaagcagccgaggttcttgcccgggtagaagctgaacgcggccaccgacgacccggcgccgatccgccggccccggtagcgggcgccgtgggcctgcgcggcgtcctcgacgatgtgcaggccgtgccggtccgcgagctcgcggagggcgtccatgtcggcggggtgcccgtagaggtggacggggaggagcgcccgggtgcggggggtgatcgccttctcgacgagcagcgggtccagggtggggtggtcctcgtgcggctcgacgggcacgggggtcgcgccggtggcggacaccgcgagccagctggcgatgtacgtgtgcgaggggacgatcacctcgtccccgggtccgatgccgaggccgcggagggcgagctggagggcgtccatcccgctgttcacgccgacggcgtggtccgtctcgcagtacgcggcgaactccgcctcgaatccttcgagttcgggtccgaggaggtagcgccccgagtcgaggacgcgggcgatcgcggcgtcggtctccgcgcggagctcctcgtaggcggccttgaggtcgaggaaggggacgcggggggtctcggcgcggctgctcacgcggacacctccacggcggtggcgggcagctgcggggcggtcgccttgagcggctcccaccagccgcggttctcccggtaccagcggacggtccgcgcgaggccgtccgcgaaggagacctgcgggcggtagccgagctcgcgctcgatctcgccgccgtcgagggagtagcgcaggtcgtggcccttgcggtcggcgaccttccggaccgaggaccagtcggcgccgagcgagtccaggaggatgccggtgagttcgcggttggtcagctccaggccgccgccgatgtggtagatctcgccggcccggccgcccgcgaggacgagcgcgatgccccggcagtggtcgtcggtgtgcacccactcgcggacgttcgcgccgtcgccgtacagcgggagcgtcccgccgtcgaggaggttcgtcacgaagagggggatgagcttctcggggtgctggtacggcccgtagttgttgcagcagcgggtgatccgtacgtcgaggccgtacgtccggtggtaggcgcgggcaacgaggtcggagccggccttggacgccgcgtagggcgagttgggctccagcgggctgctctcggtccaggagccggagtcgatcgacccgtacacctcgtcggtggagacgtgcacgacccggccgacgccggcgtcgacggcgcactggagcagcgtctgcgtgccctgcacgttggtctcggtgaacacggacgcgcccgcgatggagcggtccacgtggctctcggccgcgaagtggacgatggcgtccacgccgcgcagttcccgggcgaggaggccggcgtcgcggatgtcgccgtggacgaagcgcagtcgcgggtccgcgtccaccggggcgaggttggcgcggttgcccgcgtaggtgaggctgtccaggacgatcacctcatcggcgggcacgtcggggtacgccccggcgaggagctgccgcacgaagtgcgagccgatgaagcccgcacctccggtcaccagaagccgcactgccgtcttcctttcggtcgcgctgtaggtcgcggtgtgggtcgcactgtcggtggcggtgcgggtcgcggtgtgggtcgcactgtcggtggcgctgtcggtcgtgggaacgcgtcggccgcgaggtgccctcacggggctccctcgcggccggcgatctccatcagatagctgccgtactcggtgcgggagaggccttctcccaggccgtgacaggcctcggcgtcgatgaagcccatgcggaaggcgatctcctcaaggcccgcgatccagacgccctgccgctcctccaggacctggacgtactgggcggcccgcaggagcgagtcgtgggtgccggtgtccagccaggcgaagccgcggcccaggttgacgagttcggcccggccccgctccaggtagacgcggttgacgtcggtgatctccagctcgccgcgcggcgagggccggatgttcttggcgatgtcgacgacgtcgttgtcgtagaggtagaggccggtgacggcgaggttggagcgcggcttgacgggcttctcgacgaggtcggtcagccggcccgtcgcgtccacctcggcgacgccgtaccgctcggggtccttgaccgggtagccgaagagcacgcagccgtcgaggcgcgcgatgctgtcccgcaggagcgtgtagaggccgggcccgtggaagatgttgtcgcccaggatcagggcgcaggtgtcgtcgccgatgtgctcggctccgacgagaagtgcgtccgcgattcctgcgggctctttctggaccgcatagtcgagttctattcccaggtgcctgccgtttccgagaagcgactggaagagttcgatgtgctggggggtcgagatgatttgaatctcgcgaataccgccgagcatgagaaccgacagcggatagtagatcatcggtttgttgtagaccggaagaatctgcttcgaaatgaccgaggtcgccggatgcagccgagttccgctcccgccggccaggactattcccttcattctcggaaactagcagcagggcgccggtgataacggtcggcgtggcgagttaggggggcgctaggggctgcgcagggggagtgtcaccacccctttggggggtgggaaaacaccgagggcccggccggacggccgggccctcaggtggggggatcgtgggggggggatcggggggatcggggcgggtgcgggtcagcgcaggaagccgcgggcctcctcccagccgtccgcggcgtcgcgctccagctggttcaggcgggcggtgacgacctgatcgaagccgtccatgaagtactcgtcgccgtcgacggccgccacctcgccgccgcgctcgacgaagtccctgacgacctcggtgagggaggtgtcgggggtcacgcggcccgcgatgtagcgggtcgcgccgtccaggtcggggaagccggcctcgcggtacaggtacacgtcgccgaggagatcgacctgcaccgcgacctgcgggtgcgcggtgggccgcatggtggcgggcttgatccgcagcagttcggcgtcggccccggtgcgcaggctgttcagggcgtagccgtagtcgatgtggagtccgggggtgcgctcgcggacccgctcctcgaaggcgttgagggcctcctggagctcggcccgctcctcctgcggcagcttgccgtcgtcacggccgctgtagtcctcgcgaatgttgacgaagtcgatcgtcctgccctgcccggcgtcgttgaggtcggcgatgaagtcgaccaggtcgagcaggcgggaggcacggcccgggagcacgatgtaggcgaagccgaggttgatcggcgactcgcgctcggcgcgcagctgctggaagcggcgcaggttctcgcggacgcggcggaaggcggccttcttgccggtggtctgctcgtactcctcgtcgttgaggccgtagagcgaggtgcggatggcgtgcaggccccagaggccgggctggcgctccagggtgcgctcggtgagcgcgaaggagttcgtgtagacggtgggccgcaggccgtggtcggtggcgtgcgcggccaggctcccgaggccggggttggtgagcggctccaggccgccggagaagtacatcgccgaggggttgcccgcgggtatctcgtcgatgaccgaccggaacatggcgttgccggcgtcgagggcggacgggtcgtagcgggcgccggtcacacggacgcagaagtggcagcggaacatgcaggtcgggccggggtagaggccgacgctgtacgggaagacgggcttcctggcgagcgccgcgtcgaagacgccgcgctgttcgagcgggagcagggtgttcttccagtacgccccggcggggccggtctcgaccgcggtgcggagctccgggacctgcccgaacagggcgaggaggcgccggaaggcgtcccggtcgacgcccaggtcgtggcgggcctcctccagcggggtgaaggggctgttgccgtagcgcacggcgagccggacgaggtggcgggcggtcgttccggcctcgtcgggcggcacgaggccgccggcggcgagggtctggccgacggcgtggaccgccgcccccagatcggctccggggtgcgcgcagcgttcggccggggcggtggcggaaagggcgggggcggtcatcgggagcgtccaatcgtgggcgtggatgtctggggggccgcgagcggggcgggggccgtgtcgcggtggcgcgcggtcagttcgcggccgcgggtcgcgcagagacgcagcaggtcggcgacccggcggatgtcgtcgtcgccgatggcggtgccggtcggcagggacagcacgcgcgcggcgaggcgttcggtgtgcggcagcggggcgtgcggctgcccgcggtacggctccagctcgtggcagcccggcgagaagtaggcgcgggtgtgcacgccttcggccttcaggacctccatgacgaggtcgcggtggatgccggtggtggcctcgtcgatctcgacgatcacgtactggtggttgttgaggccgtggcggtcgtggtcggcgacgaggacgccggggaggtccgcgaggtgctcgcggtaggcggcgtggttgcgccggttccggtcgatgacctcgggaaacgcgtcgagggaggtgaggcccatggcggcggcggcctcgctcatcttggcgttggtcccgccggcggggctgccgccgggcaggtcgaagccgaagttgtggagggcgcggatccgggcggcgaggtcggcgtcgtcggtgacgacggcgccgccctcgaaggcgttgacggccttggtggcgtggaagctgaagacctcggcgtcgccgaggctgccggcgggccggccgtcgaccgcgcagccgagggcgtgcgcggcgtcgaagtacagccgcaggccgtgctcgtcggcgaccttccgcagctggtcggcggcgcaggggcggccccagaggtggacgccgacgacggccgaggtgcggggtgtgaccgcggcggccacctggtccgggtcgaggttgccggtgtccgggtcgatgtcggcgaagaccggggtgaggccgatccagcgcagtgcgtgcggggtggcggcgaacgtcatcgacggcatgatcacttcgccggtgaggccggcggcgtgcgcgaggagctggagcccggccgtggcgttgcaggtggccacggcatgccggaccccggcgagcccggcgacgcgctcctcgaactcgcggacgagcgggccgccgttggacagccactggctgtcgagggcccggtcgagccgctcgtacagcctggcgcggtcgatgcggttgggccgccccacgaggagcggctggtcgaaagcggcggggccgccgaagaatgcgaggtcggataaggcgcttttcacggatgttccctccgggccaccgtcacgaaatgattcgccgatccgggaatcccgaacgaggtcgccgcgctccaccgtgacgtacgacgagatggtcgattgtggtggtcgatttcggggggactctaatccgcgcggaacgggaccgacaagagcacgctatgcgctctcgatgtgcttcggatcacatccgcctccggggtattccatcggcggcccgaatgtgatgatccttgacaggatcc

SEQ ID NO: 4 - protein, 3782 aa, Streptomyces venezuelae
MTDDLTGALTQPPLGRTVRAVADRELGTHLLETRGIHWIHAANGDPYATVLRGQADDPYPAYERVRARGALSFSPTGSWVTADHALAASILCSTDFGVSGADGVPVPQQVLSYGEGCPLEREQVLPAAGDVPEGGQRAVVEGIHRETLEGLAPDPSASYAFELLGGFVRPAVTAAAAAVLGVPADRRADFADLLERLRPLSDSLLAPQSLRTVRAADGALAELTALLADSDDSPGALLSALGVTAAVQLTGNAVLALLAHPEQWRELCDRPGLAAAAVEETLRYDPPVQLDARVVRGETELAGRRLPAGAHVVVLTAATGRDPEVFTDPERFDLARPDAAAHLALHPAGPYGPVASLVRLQAEVALRTLAGRFPGLRQAGDVLRPRRAPVGRGPLSVPVSSSMRVLLTSFAHHTHYYGLVPLAWALLAAGHEVRVASQPALTDTITGSGLAAVPVGTDHLIHEYRVRMAGEPRPNHPAIAFDEARPEPLDWDHALGIEAILAPYFHLLANNDSMVDDLVDFARSWQPDLVLWEPTTYAGAVAAQVTGAAHARVLWGPDVMGSARRKFVALRDRQPPEHREDPTAEWLTWTLDRYGASFEEELLTGQFTIDPTPPSLRLDTGLPTVGMRYVPYNGTSVVPDWLSEPPARPRVCLTLGVSAREVLGGDGVSQGDILEALADLDIELVATLDASQRAEIRNYPKHTRFTDFVPMHALLPSCSAIIHHGGAGTYATAVINAVPQVMLAELWDAPVKARAVAEQGAGFFLPPAELTPQAVRDAVVRILDDPSVATAAHRLREETFGDPTPAGIVPELERLAAQHRRPPADARHMYEVDHADVYDLFYLGRGKDYAAEASDIADLVRSRTPEASSLLDVACGTGTHLEHFTKEFGDTAGLELSEDMLTHARKRLPDATLHQGDMRDFRLGRKFSAVVSMFSSVGYLKTTEELGAAVASFAEHLEPGGVVVVEPWWFPETFADGWVSADVVRRDGRTVARVSHSVREGNATRMEVHFTVADPGKGVRHFSDVHLITLFHQAEYEAAFTAAGLRVEYLEGGPSGRGLFVGVPAMTGKTRIPRVRRGRTTPRAFTLAVVGTLLAGTTVAAAAPGAADTANVQYTSRAAELVAQMTLDEKISFVHWALDPDRQNVGYLPGVPRLGIPELRAADGPNGIRLVGQTATALPAPVALASTFDDTMADSYGKVMGRDGRALNQDMVLGPMMNNIRVPHGGRNYETFSEDPLVSSRTAVAQIKGIQGAGLMTTAKHFAANNQENNRFSVNANVDEQTLREIEFPAFEASSKAGAASFMCAYNGLNGKPSCGNDELLNNVLRTQWGFQGWVMSDWLATPGTDAITKGLDQEMGVELPGDVPKGEPSPPAKFFGEALKTAVLNGTVPEAAVTRSAERIVGQMEKFGLLLATPAPRPERDKAGAQAVSRKVAENGAVLLRNEGQALPLAGDAGKSIAVIGPTAVDPKVTGLGSAHVVPDSAAAPLDTIKARAGAGATVTYETGEETFGTQIPAGNLSPAFNQGHQLEPGKAGALYDGTLTVPADGEYRIAVRATGGYATVQLGSHTIEAGQVYGKVSSPLLKLTKGTHKLTISGFAMSATPLSLELGWVTPAAADATIAKAVESARKARTAVVFAYDDGTEGVDRPNLSLPGTQDKLISAVADANPNTIVVLNTGSSVLMPWLSKTRAVLDMWYPGQAGAEATAALLYGDVNPSGKLTQSFPAAENQHAVAGDPTSYPGVDNQQTYREGIHVGYRWFDKENVKPLFPFGHGLSYTSFTQSAPTVVRTSTGGLKVTVTVRNSGKRAGQEVVQAYLGASPNVTAPQAKKKLVGYTKVSLAAGEAKTVTVNVDRRQLQTGSSSADLRGSATVNVWMSSRAETPRVPFLDLKAAYEELRAETDAAIARVLDSGRYLLGPELEGFEAEFAAYCETDHAVGVNSGMDALQLALRGLGIGPGDEVIVPSHTYIASWLAVSATGATPVPVEPHEDHPTLDPLLVEKAITPRTRALLPVHLYGHPADMDALRELADRHGLHIVEDAAQAHGARYRGRRIGAGSSVAAFSFYPGKNLGCFGDGGAVVTGDPELAERLRMLRNYGSRQKYSHETKGTNSRLDEMQAAVLRIRLAHLDSWNGRRSALAAEYLSGLAGLPGIGLPVTAPDTDPVWHLFTVRTERRDELRSHLDARGIDTLTHYPVPVHLSPAYAGEAPPEGSLPRAESFARQVLSLPIGPHLERPQALRVIDAVREWAERVDQAMRLLVTGGAGFIGSHFVRQLLAGAYPDVPADEVIVLDSLTYAGNRANLAPVDADPRLRFVHGDIRDAGLLARELRGVDAIVHFAAESHVDRSIAGASVFTETNVQGTQTLLQCAVDAGVGRVVHVSTDEVYGSIDSGSWTESSPLEPNSPYAASKAGSDLVARAYHRTYGLDVRITRCCNNYGPYQHPEKLIPLFVTNLLDGGTLPLYGDGANVREWVHTDDHCRGIALVLAGGRAGEIYHIGGGLELTNRELTGILLDSLGADWSSVRKVADRKGHDLRYSLDGGEIERELGYRPQVSFADGLARTVRWYRENRGWWEPLKATAPQLPATAVEVSAMKGIVLAGGSGTRLHPATSVISKQILPVYNKPMIYYPLSVLMLGGIREIQIISTPQHIELFQSLLGNGRHLGIELDYAVQKEPAGIADALLVGAEHIGDDTCALILGDNIFHGPGLYTLLRDSIARLDGCVLFGYPVKDPERYGVAEVDATGRLTDLVEKPVKPRSNLAVTGLYLYDNDVVDIAKNIRPSPRGELEITDVNRVYLERGRAELVNLGRGFAWLDTGTHDSLLRAAQYVQVLEERQGVWIAGLEEIAFRMGFIDAEACHGLGEGLSRTEYGSYLMEIAGREGAPMTAPALSATAPAERCAHPGADLGAAVHAVGQTLAAGGLVPPDEAGTTARHLVRLAVRYGNSPFTPLEEARHDLGVDRDAFRRLLALFGQVPELRTAVETGPAGAYWKNTLLPLEQRGVFDAALARKPVFPYSVGLYPGPTCMFRCHFCVRVTGARYDPSALDAGNAMFRSVIDEIPAGNPSAMYFSGGLEPLTNPGLGSLAAHATDHGLRPTVYTNSFALTERTLERQPGLWGLHAIRTSLYGLNDEEYEQTTGKKAAFRRVRENLRRFQQLRAERESPINLGFAYIVLPGRASRLLDLVDFIADLNDAGQGRTIDFVNIREDYSGRDDGKLPQEERAELQEALNAFEERVRERTPGLHIDYGYALNSLRTGADAELLRIKPATMRPTAHPQVAVQVDLLGDVYLYREAGFPDLDGATRYIAGRVTPDTSLTEVVRDFVERGGEVAAVDGDEYFMDGFDQVVTARLNQLERDAADGWEEARGFLRMKSALSDLAFFGGPAAFDQPLLVGRPNRIDRARLYERLDRALDSQWLSNGGPLVREFEERVAGLAGVRHAVATCNATAGLQLLAHAAGLTGEVIMPSMTFAATPHALRWIGLTPVFADIDPDTGNLDPDQVAAAVTPRTSAVVGVHLWGRPCAADQLRKVADEHGLRLYFDAAHALGCAVDGRPAGSLGDAEVFSFHATKAVNAFEGGAVVTDDADLAARIRALHNFGFDLPGGSPAGGTNAKMSEAAAAMGLTSLDAFPEVIDRNRRNHAAYREHLADLPGVLVADHDRHGLNNHQYVIVEIDEATTGIHRDLVMEVLKAEGVHTRAYFSPGCHELEPYRGQPHAPLPHTERLAARVLSLPTGTAIGDDDIRRVADLLRLCATRGRELTARHRDTAPAPLAAPQTSTPTIGRSR

SEQ ID NO: 5 - DNA, 37948 bp, Streptomyces venezuelae
gggcccctcctcacgcgtctcgatcctcgcgcgtccgcgccttcccgcgccggcactcgcgctctcgcgttcgttcaggccctccgcttccgggtcccgccggtgcggctcttcgtgctgctccggctccgaacggtttcgcggagcagactcatggcattttccccgcagggcggccgacacgagctcggtcagaacttcctcgtcgaccggtcagtgatcgacgagatcgacggcctggtggccaggaccaagggtccgatactggagatcggtccgggtgacggcgccctgaccctgccgctgagcaggcacggcaggccgatcaccgccgtcgagctcgacggccggcgcgcgcagcgcctcggtgcccgcacccccggtcatgtgaccgtggtgcaccacgacttcctgcagtacccgctgccgcgcaacccgcatgtggtcgtcggcaacgtccccttccatctgacgacggcgatcatgcggcggctgctcgacgcccagcactggcacaccgccgtcctcctcgtccagtgggaggtcgcccggcgccgggccggcgtcggcgggtcgacgctgctgacggccggctgggcgccctggtacgagttcgacctgcactcccgggtccccgcgcgggccttccgtccgatgccgggcgtggacggaggagtactggccatccggcggcggtccgcgccgctcgtgggccaggtgaagacgtaccaggacttcgtacgccaggtgttcaccggcaaggggaacgggctgaaggagatcctgcggcggaccgggcggatctcgcagcgggacctggcgacctggctgcggaggaacgagatctcgccgcacgcgctgcccaaggacctgaagcccgggcagtgggcgtcgctgtgggagctgaccggcggcacggccgacggatccttcgacggtacggcgggcggtggcgcggccggatcgcacggggcggctcgggtcggggccggtcacccgggcggccgggtgtccgcgagccggcggggcgtgccgcaggcgcggcgcggccgggggcatgcggtacggagctccacggggaccgagccgaggtggggcagggggcgggcggagagcgcgtgagccgttctcgagcctgctgccgagccgctgctgagccggtgctgagccggatccgaccgtgggtgtgaatctccgggtgctcgcctcgtcctgccccgttacctgtccgcctcccgctccagaccagcgggaggcggacaggggcatgcccgccgggcggctaacggcccgtgcggcgtccgtacgacgagcctcgcgcgccctggcggcccttggtctgccggacctgtgcgcggggtgcgcagggttcgccgccgcgcgtggggccgtatctgcggctcccgggcacggcggccctgctcgtctccgagtcatagtccctgccgccggcgccaccgccctggcccggcatgcgcgtgccgggcgcccccggcgcgtaactcggctgggaggcctggaaaagggcgatccattgggtgagcgtgaggtccttcggcagtccgccgtccggaattccgtggcggtcggcgagggaacggtaggtccgcttggggatgtggcgccggaggatctccgcgaggccccgtccggggccggtgaagacggcttcggcgaagttctggaaggcgcggctcgcgctctcgggcagcaggggctgggggcgtcgcctgatcgtcaggacgccgccgtcgacgcggggcatcggacggaacgacgaggcgcggacgcggtcgtggaccgcgaactcgtaccagggggcccaggaggtcgtgaggagcgatccgccgctgcgaccggcgcgtttgcgggcgacctcccactgcactatcagggccgccgactgccagttcgtcgattccaggagactccggagaatctgggtcgtgatgccgaagggaacgtttccgacgacggtgtcgatatcgcgcggaatgcggaagtcgaggaaatcaccctggaatacggtgaccctctccccttcgaatttccgccgcacatgcgcggcccagtgcgggtccatctccacgaccgtcacggtgtcgaaggagcgcaccaactcctcggttatcgcgccctttccggggccgatttcgagaacgttcctaccgtccccctcgacatgcgtgacgagattgcgcacggctctgtcgtcctgaaggaagttctggcctaattcgcggcgaagggtgtcgcggtccgctcgcctcggtatggagtcgcgcattgccatgaacgatcccctccctggatgccgtggtcaatggacttggcacggaccatacctcacggtccgtcggacgaccggagaagaagttcacgcacgggcgttccggagtacgggagttgtgaacggccgcgacgaagtcggtcgcggctcggcgggcggtgacgagcgaggtccggaggaacgcgacgaagcagccgaaccccaagtgaggtgcgacggagtgacattgggggcatacggagggttgtcgtacggagcgcactcaacgaggctccaggagggaggggttgaacccgccgccgactggccttcgccgcccgcgcggccggagtatgtcatgtcgggggtgaaatcaagccattcccccgggatcggctgttacccatccctttacctggcgtggatttcccaacccttggtatagagcgggagacgacgcgacaccatggagaccacgcacaccacgagcgccaccccccggccatcccgacaaggggggtccggctcgcctcccgacacccatggcctggggtacacgccaggtatagggggaacgtagggggagcatagggggggtgccctggggttgggtgaaagcgcggcttccggagacggagccggatgtcttcagccggaattaccaggaccggtgcgagaacaccggtgacagggcgtggggcggcagcgtgggacacgggggaagtgcgggtccgacgggggttgccccctgccggccccgatcatgcggagcactccttctctcgtgctcctaccggtgatgtgcgcgccgaattgattcgtggagagatgtcgacagtgtccaagagtgagtccgaggaattcgtgtccgtgtcgaacgacgccggttccgcgcacggcacagcggaacccgtcgccgtcgtcggcatctcctgccgggtgcccggcgcccgggacccgagagagttctgggaactcctggcggcaggcggccaggccgtcaccgacgtccccgcggaccgctggaacgccggcgacttctacgacccggaccgctccgcccccggccgctcgaacagccggtggggcgggttcatcgaggacgtcgaccggttcgacgccgccttcttcggcatctcgccccgcgaggccgcggagatggacccgcagcagcggctcgccctggagctgggctgggaggccctggagcgcgccgggatcgacccgtcctcgctcaccggcacccgcaccggcgtcttcgccggcgccatctgggacgactacgccaccctgaagcaccgccagggcggcgccgcgatcaccccgcacaccgtcaccggcctccaccgcggcatcatcgcgaaccgactctcgtacacgctcgggctccgcggccccagcatggtcgtcgactccggccagtcctcgtcgctcgtcgccgtccacctcgcgtgcgagagcctgcggcgcggcgagtccgagctcgccctcgccggcggcgtctcgctcaacctggtgccggacagcatcatcggggcgagcaagttcggcggcctctcccccgacggccgcgcctacaccttcgacgcgcgcgccaacggctacgtacgcggcgagggcggcggtttcgtcgtcctgaagcgcctctcccgggccgtcgccgacggcgacccggtgctcgccgtgatccggggcagcgccgtcaacaacggcggcgccgcccagggcatgacgacccccgacgcgcaggcgcaggaggccgtgctccgcgaggcccacgagcgggccgggaccgcgccggccgacgtgcggtacgtcgagctgcacggcaccggcacccccgtgggcgacccgatcgaggccgctgcgctcggcgccgccctcggcaccggccgcccggccggacagccgctcctggtcggctcggtcaagacgaacatcggccacctggagggcgcggccggcatcgccggcctcatcaaggccgtcctggcggtccgcggtcgcgcgctgcccgccagcctgaactacgagaccccgaacccggcgatcccgttcgaggaactgaacctccgggtgaacacggagtacctgccgtgggagccggagcacgacgggcagcggatggtcgtcggcgtgtcctcgttcggcatgggcggcacgaacgcgcatgtcgtgctcgaagaggcccccgggggttgtcgaggtgcttcggtcgtggagtcgacggtcggcgggtcggcggtcggcggcggtgtggtgccgtgggtggtgtcggcgaagtccgctgccgcgctggacgcgcagatcgagcggcttgccgcgttcgcctcgcgggatcgtacggatggtgtcgacgcgggcgctgtcgatgcgggtgctgtcgatgcgggtgctgtcgctcgcgtactggccggcgggcgtgctcagttcgagcaccgggccgtcgtcgtcggcagcgggccggacgatctggcggcagcgctggccgcgcctgagggtctggtccggggcgtggcttccggtgtcgggcgagtggcgttcgtgttccccgggcagggcacgcagtgggccggcatgggtgccgaactgctggactcttccgcggtgttcgcggcggccatggccgaatgcgaggccgcactctccccgtacgtcgactggtcgctggaggccgtcgtacggcaggcccccggtgcgcccacgctggagcgggtcgatgtcgtgcagcctgtgacgttcgccgtcatggtctcgctggctcgcgtgtggcagcaccacggggtgacgccccaggcggtcgtcggccactcgcagggcgagatcgccgccgcgtacgtcgccggtgccctgagcctggacgacgccgctcgtgtcgtgaccctgcgcagcaagtccatcgccgcccacctcgccggcaagggcggcatgctgtccctcgcgctgagcgaggacgccgtcctggagcgactggccgggttcgacgggctgtccgtcgccgctgtgaacgggcccaccgccaccgtggtctccggtgaccccgtacagatcgaagagcttgctcgggcgtgtgaggccgatggggtccgtgcgcgggtcattcccgtcgactacgcgtcccacagccggcaggtcgagatcatcgagagcgagctcgccgaggtcctcgccgggctcagcccgcaggctccgcgcgtgccgttcttctcgacactcgaaggcgcctggatcaccgagcccgtgctcgacggcggctactggtaccgcaacctgcgccatcgtgtgggcttcgccccggccgtcgagaccctggccaccgacgagggcttcacccacttcgtcgaggtcagcgcccaccccgtcctcaccatggccctccccgggaccgtcaccggtctggcgaccctgcgtcgcgacaacggcggtcaggaccgcctagtcgcctccctcgccgaagcatgggccaacggactcgcggtcgactggagcccgctcctcccctccgcgaccggccaccactccgacctccccacctacgcgttccagaccgagcgccactggctgggcgagatcgaggcgctcgccccggcgggcgagccggcggtgcagcccgccgtcctccgcacggaggcggccgagccggcggagctcgaccgggacgagcagctgcgcgtgatcctggacaaggtccgggcgcagacggcccaggtgctggggtacgcgacaggcgggcagatcgaggtcgaccggaccttccgtgaggccggttgcacctccctgaccggcgtggacctgcgcaaccggatcaacgccgccttcggcgtacggatggcgccgtccatgatcttcgacttccccacccccgaggctctcgcggagcagctgctcctcgtcgtgcacggggaggcggcggcgaacccggccggtgcggagccggctccggtggcggcggccggtgccgtcgacgagccggtggcgatcgtcggcatggcctgccgcctgcccggtggggtcgcctcgccggaggacctgtggcggctggtggccggcggcggggacgcgatctcggagttcccgcaggaccgcggctgggacgtggaggggctgtaccacccggatccggagcaccccggcacgtcgtacgtccgccagggcggtttcatcgagaacgtcgccggcttcgacgcggccttcttcgggatctcgccgcgcgaggccctcgccatggacccgcagcagcggctcctcctcgaaacctcctgggaggccgtcgaggacgccgggatcgacccgacctccctgcggggacggcaggtcggcgtcttcactggggcgatgacccacgagtacgggccgagcctgcgggacggcggggaaggcctcgacggctacctgctgaccggcaacacggccagcgtgatgtcgggccgcgtctcgtacacactcggccttgagggccccgccctgacggtggacacggcctgctcgtcgtcgctggtcgccctgcacctcgccgtgcaggccctgcgcaagggcgaggtcgacatggcgctcgccggcggcgtggccgtgatgcccacgcccgggatgttcgtcgagttcagccggcagcgcgggctggccggggacggccggtcgaaggcgttcgccgcgtcggcggacggcaccagctggtccgagggcgtcggcgtcctcctcgtcgagcgcctgtcggacgcccgccgcaacggacaccaggtcctcgcggtcgtccgcggcagcgccttgaaccaggacggcgcgagcaacggcctcacggctccgaacgggccctcgcagcagcgcgtcatccggcgcgcgctggcggacgcccggctgacgacctccgacgtggacgtcgtcgaggcacacggcacgggcacgcgactcggcgacccgatcgaggcgcaggccctgatcgccacctacggccagggccgtgacgacgaacagccgctgcgcctcgggtcgttgaagtccaacatcgggcacacccaggccgcggccggcgtctccggtgtcatcaagatggtccaggcgatgcgccacggactgctgccgaagacgctgcacgtcgacgagccctcggaccagatcgactggtcggctggcgccgtggaactcctcaccgaggccgtcgactggccggagaagcaggacggcgggctgcgccgggccgccgtctcctccttcgggatcagcggcaccaatgcgcatgtggtgctcgaagaggccccggtggttgtcgagggtgcttcggtcgtcgagccgtcggttggcgggtcggcggtcggcggcggtgtgacgccttgggtggtgtcggcgaagtccgctgccgcgctcgacgcgcagatcgagcggcttgccgcattcgcctcgcgggatcgtacggatgacgccgacgccggtgctgtcgacgcgggcgctgtcgctcacgtactggctgacgggcgtgctcagttcgagcaccgggccgtcgcgctcggcgccggggcggacgacctcgtacaggcgctggccgatccggacgggctgatacgcggaacggcttccggtgtcgggcgagtggcgttcgtgttccccggtcagggcacgcagtgggctggcatgggtgccgaactgctggactcttccgcggtgttcgcggcggccatggccgagtgtgaggccgcgctgtccccgtacgtcgactggtcgctggaggccgtcgtacggcaggcccccggtgcgcccacgctggagcgggtcgatgtcgtgcagcctgtgacgttcgccgtcatggtctcgctggctcgcgtgtggcagcaccacggtgtgacgccccaggcggtcgtcggccactcgcagggcgagatcgccgccgcgtacgtcgccggagccctgcccctggacgacgccgcccgcgtcgtcaccctgcgcagcaagtccatcgccgcccacctcgccggcaagggcggcatgctgtccctcgcgctgaacgaggacgccgtcctggagcgactgagtgacttcgacgggctgtccgtcgccgccgtcaacgggcccaccgccactgtcgtgtcgggtgaccccgtacagatcgaagagcttgctcaggcgtgcaaggcggacggattccgcgcgcggatcattcccgtcgactacgcgtcccacagccggcaggtcgagatcatcgagagcgagctcgcccaggtcctcgccggtctcagcccgcaggccccgcgcgtgccgttcttctcgacgctcgaaggcacctggatcaccgagcccgtcctcgacggcacctactggtaccgcaacctccgtcaccgcgtcggcttcgcccccgccatcgagaccctggccgtcgacgagggcttcacgcacttcgtcgaggtcagcgcccaccccgtcctcaccatgaccctccccgagaccgtcaccggcctcggcaccctccgtcgcgaacagggaggccaagagcgtctggtcacctcgctcgccgaggcgtgggtcaacgggcttcccgtggcatggacttcgctcctgcccgccacggcctcccgccccggtctgcccacctacgccttccaggccgagcgctactggctcgagaacactcccgccgccctggccaccggcgacgactggcgctaccgcatcgactggaagcgcctcccggccgccgaggggtccgagcgcaccggcctgtccggccgctggctcgccgtcacgccggaggaccactccgcgcaggccgccgccgtgctcaccgcgctggtcgacgccggggcgaaggtcgaggtgctgacggccggggcggacgacgaccgtgaggccctcgccgcccggctcaccgcactgacgaccggtgacggcttcaccggcgtggtctcgctcctcgacggactcgtaccgcaggtcgcctgggtccaggcgctcggcgacgccggaatcaaggcgcccctgtggtccgtcacccagggcgcggtctccgtcggacgtctcgacacccccgccgaccccgaccgggccatgctctggggcctcggccgcgtcgtcgcccttgagcaccccgaacgctgggccggcctcgtcgacctccccgcccagcccgatgccgccgccctcgcccacctcgtcaccgcactctccggcgccaccggcgaggaccagatcgccatccgcaccaccggactccacgcccgccgcctcgcccgcgcacccctccacggacgtcggcccacccgcgactggcagccccacggcaccgtcctcatcaccggcggcaccggagccctcggcagccacgccgcacgctggatggcccaccacggagccgaacacctcctcctcgtcagccgcagcggcgaacaagcccccggagccacccaactcaccgccgaactcaccgcatcgggcgcccgcgtcaccatcgccgcctgcgacgtcgccgacccccacgccatgcgcaccctcctcgacgccatccccgccgagacgcccctcaccgccgtcgtccacaccgccggcgcgctcgacgacggcatcgtggacacgctgaccgccgagcaggtccggcgggcccaccgtgcgaaggccgtcggcgcctcggtgctcgacgagctgacccgggacctcgacctcgacgcgttcgtgctcttctcgtccgtgtcgagcactctgggcatccccggtcagggcaactacgccccgcacaacgcctacctcgacgccctcgcggctcgccgccgggccaccggccggtccgccgtctcggtggcctggggaccgtgggacggtggcggcatggccgccggtgacggcgtggccgagcggctgcgcaaccacggcgtgcccggcatggacccggaactcgccctggccgcactggagtccgcgctcggccgggacgagaccgcgatcaccgtcgcggacatcgactgggaccgcttctacctcgcgtactcctccggtcgcccgcagcccctcgtcgaggagctgcccgaggtgcggcgcatcatcgacgcacgggacagcgccacgtccggacagggcgggagctccgcccagggcgccaaccccctggccgagcggctggccgccgcggctcccggcgagcgtacggagatcctcctcggtctcgtacgggcgcaggccgccgccgtgctccggatgcgttcgccggaggacgtcgccgccgaccgcgccttcaaggacatcggcttcgactcgctcgccggtgtcgagctgcgcaacaggctgacccgggcgaccgggctccagctgcccgcgacgctcgtcttcgaccacccgacgccgctggccctcgtgtcgctgctccgcagcgagttcctcggtgacgaggagacggcggacgcccggcggtccgcggcgctgcccgcgactgtcggtgccggtgccggcgccggcgccggcaccgatgccgacgacgatccgatcgcgatcgtcgcgatgagctgccgctaccccggtgacatccgcagcccggaggacctgtggcggatgctgtccgagggcggcgagggcatcacgccgttccccaccgaccgcggctgggacctcgacggcctgtacgacgccgacccggacgcgctcggcagggcgtacgtccgcgagggcgggttcctgcacgacgcggccgagttcgacgcggagttcttcggcgtctcgccgcgcgaggcgctggccatggacccgcagcagcggatgctcctgacgacgtcctgggaggccttcgagcgggccggcatcgagccggcatcgctgcgcggcagcagcaccggtgtcttcatcggcctctcctaccaggactacgcggcccgcgtcccgaacgccccgcgtggcgtggagggttacctgctgaccggcagcacgccgagcgtcgcgtcgggccgtatcgcgtacaccttcggtctcgaagggcccgcgacgaccgtcgacaccgcctgctcgtcgtcgctgaccgccctgcacctggcggtgcgggcgctgcgcagcggcgagtgcacgatggcgctcgccggtggcgtggcgatgatggcgaccccgcacatgttcgtggagttcagccgtcagcgggcgctcgccccggacggccgcagcaaggccttctcggcggacgccgacgggttcggcgccgcggagggcgtcggcctgctgctcgtggagcggctctcggacgcgcggcgcaacggtcacccggtgctcgccgtggtccgcggtaccgccgtcaaccaggacggcgccagcaacgggctgaccgcgcccaacggaccctcgcagcagcgggtgatccggcaggcgctcgccgacgcccggctggcacccggcgacatcgacgccgtcgagacgcacggcacgggaacctcgctgggcgaccccatcgaggcccagggcctccaggccacgtacggcaaggagcggcccgcggaacggccgctcgccatcggctccgtgaagtccaacatcggacacacccaggccgcggccggtgcggcgggcatcatcaagatggtcctcgcgatgcgccacggcaccctgccgaagaccctccacgccgacgagccgagcccgcacgtcgactgggcgaacagcggcctggccctcgtcaccgagccgatcgactggccggccggcaccggtccgcgccgcgccgccgtctcctccttcggcatcagcgggacgaacgcgcacgtcgtgctggagcaggcgccggatgctgctggtgaggtgcttggggccgatgaggtgcctgaggtgtctgagacggtagcgatggctgggacggctgggacctccgaggtcgctgagggctctgaggcctccgaggcccccgcggcccccggcagccgtgaggcgtccctccccgggcacctgccctgggtgctgtccgccaaggacgagcagtcgctgcgcggccaggccgccgccctgcacgcgtggctgtccgagcccgccgccgacctgtcggacgcggacggaccggcccgcctgcgggacgtcgggtacacgctcgccacgagccgtaccgccttcgcgcaccgcgccgccgtgaccgccgccgaccgggacgggttcctggacgggctggccacgctggcccagggcggcacctcggcccacgtccacctggacaccgcccgggacggcaccaccgcgttcctcttcaccggccagggcagtcagcgccccggcgccggccgtgagctgtacgaccggcaccccgtcttcgcccgggcgctcgacgagatctgcgcccacctcgacggtcacctcgaactgcccctgctcgacgtgatgttcgcggccgagggcagcgcggaggccgcgctgctcgacgagacgcggtacacgcagtgcgcgctgttcgccctggaggtcgcgctcttccggctcgtcgagagctggggcatgcggccggccgcactgctcggtcactcggtcggcgagatcgccgccgcgcacgtcgccggtgtgttctcgctcgccgacgccgcccgcctggtcgccgcgcgcggccggctcatgcaggagctgcccgccggtggcgcgatgctcgccgtccaggccgcggaggacgagatccgcgtgtggctggagacggaggagcggtacgcgggacgtctggacgtcgccgccgtcaacggccccgaggccgccgtcctgtccggcgacgcggacgcggcgcgggaggcggaggcgtactggtccgggctcggccgcaggacccgcgcgctgcgggtcagccacgccttccactccgcgcacatggacggcatgctcgacgggttccgcgccgtcctggagacggtggagttccggcgcccctccctgaccgtggtctcgaacgtcaccggcctggccgccggcccggacgacctgtgcgaccccgagtactgggtccggcacgtccgcggcaccgtccgcttcctcgacggcgtccgtgtcctgcgcgacctcggcgtgcggacctgcctggagctgggccccgacggggtcctcaccgccatggcggccgacggcctcgcggacacccccgcggattccgctgccggctcccccgtcggctctcccgccggctctcccgccgactccgccgccggcgcgctccggccccggccgctgctcgtggcgctgctgcgccgcaagcggtcggagaccgagaccgtcgcggacgccctcggcagggcgcacgcccacggcaccggacccgactggcacgcctggttcgccggctccggggcgcaccgcgtggacctgcccacgtactccttccggcgcgaccgctactggctggacgccccggcggccgacaccgcggtggacaccgccggcctcggtctcggcaccgccgaccacccgctgctcggcgccgtggtcagccttccggaccgggacggcctgctgctcaccggccgcctctccctgcgcacccacccgtggctcgcggaccacgccgtcctggggagcgtcctgctccccggcgccgcgatggtcgaactcgccgcgcacgctgcggagtccgccggtctgcgtgacgtgcgggagctgaccctccttgaaccgctggtactgcccgagcacggtggcgtcgagctgcgcgtgacggtcggggcgccggccggagagcccggtggcgagtcggccggggacggcgcacggcccgtctccctccactcgcggctcgccgacgcgcccgccggtaccgcctggtcctgccacgcgaccggtctgctggccaccgaccggcccgagcttcccgtcgcgcccgaccgtgcggccatgtggccgccgcagggcgccgaggaggtgccgctcgacggtctctacgagcggctcgacgggaacggcctcgccttcggtccgctgttccaggggctgaacgcggtgtggcggtacgagggtgaggtcttcgccgacatcgcgctccccgccaccacgaatgcgaccgcgcccgcgaccgcgaacggcggcgggagtgcggcggcggccccctacggcatccaccccgccctgctcgacgcttcgctgcacgccatcgcggtcggcggtctcgtcgacgagcccgagctcgtccgcgtccccttccactggagcggtgtcaccgtgcacgcggccggtgccgcggcggcccgggtccgtctcgcctccgcggggacggacgccgtctcgctgtccctgacggacggcgagggacgcccgctggtctccgtggaacggctcacgctgcgcccggtcaccgccgatcaggcggcggcgagccgcgtcggcgggctgatgcaccgggtggcctggcgtccgtacgccctcgcctcgtccggcgaacaggacccgcacgccacttcgtacgggccgaccgccgtcctcggcaaggacgagctgaaggtcgccgccgccctggagtccgcgggcgtcgaagtcgggctctaccccgacctggccgcgctgtcccaggacgtggcggccggcgccccggcgccccgtaccgtccttgcgccgctgcccgcgggtcccgccgacggcggcgcggagggtgtacggggcacggtggcccggacgctggagctgctccaggcctggctggccgacgagcacctcgcgggcacccgcctgctcctggtcacccgcggtgcggtgcgggaccccgaggggtccggcgccgacgatggcggcgaggacctgtcgcacgcggccgcctggggtctcgtacggaccgcgcagaccgagaaccccggccgcttcggccttctcgacctggccgacgacgcctcgtcgtaccggaccctgccgtcggtgctctccgacgcgggcctgcgcgacgaaccgcagctcgccctgcacgacggcaccatcaggctggcccgcctggcctccgtccggcccgagaccggcaccgccgcaccggcgctcgccccggagggcacggtcctgctgaccggcggcaccggcggcctgggcggactggtcgcccggcacgtggtgggcgagtggggcgtacgacgcctgctgctggtgagccggcggggcacggacgccccgggcgccgacgagctcgtgcacgagctggaggccctgggagccgacgtctcggtggccgcgtgcgacgtcgccgaccgcgaagccctcaccgccgtactcgacgccatccccgccgaacacccgctcaccgcggtcgtccacacggcaggcgtcctctccgacggcaccctcccgtccatgacgacggaggacgtggaacacgtactgcggcccaaggtcgacgccgcgttcctcctcgacgaactcacctcgacgcccgcatacgacctggcagcgttcgtcatgttctcctccgccgccgccgtcttcggtggcgcggggcagggcgcctacgccgccgccaacgccaccctcgacgccctcgcctggcgccgccgggcagccggactccccgccctctccctcggctggggcctctgggccgagaccagcggcatgaccggcgagctcggccaggcggacctgcgccggatgagccgcgcgggcatcggcgggatcagcgacgccgagggcatcgcgctcctcgacgccgccctccgcgacgaccgccacccggtcctgctgcccctgcggctcgacgccgccgggctgcgggacgcggccgggaacgacccggccggaatcccggcgctcttccgggacgtcgtcggcgccaggaccgtccgggcccggccgtccgcggcctccgcctcgacgacagccgggacggccggcacgccggggacggcggacggcgcggcggaaacggcggcggtcacgctcgccgaccgggccgccaccgtggacgggcccgcacggcagcgcctgctgctcgagttcgtcgtcggcgaggtcgccgaagtactcggccacgcccgcggtcaccggatcgacgccgaacggggcttcctcgacctcggcttcgactccctgaccgccgtcgaactccgcaaccggctcaactccgccggtggcctcgccctcccggcgaccctggtcttcgaccacccaagcccggcggcactcgcctcccacctggacgccgagctgccgcgcggcgcctcggaccaggacggagccgggaaccggaacgggaacgagaacgggacgacggcgtcccggagcaccgccgagacggacgcgctgctggcacaactgacccgcctggaaggcgccttggtgctgacgggcctctcggacgcccccgggagcgaagaagtcctggagcacctgcggtccctgcgctcgatggtcacgggcgagaccgggaccgggaccgcgtccggagccccggacggcgccgggtccggcgccgaggaccggccctgggcggccggggacggagccgggggcgggagtgaggacggcgcgggagtgccggacttcatgaacgcctcggccgaggaactcttcggcctcctcgaccaggaccccagcacggactgatccctgccgcacggtcgcctcccgccccggaccccgtcccgggcacctcgactcgaatcacttcatgcgcgcctcgggcgcctccaggaactcaaggggacagcgtgtccacggtgaacgaagagaagtacctcgactacctgcgtcgtgccacggcggacctccacgaggcccgtggccgcctccgcgagctggaggcgaaggcgggcgagccggtggcgatcgtcggcatggcctgccgcctgcccggcggcgtcgcctcgcccgaggacctgtggcggctggtggccggcggcgaggacgcgatctcggagttcccccaggaccgcggctgggacgtggagggcctgtacgacccgaacccggaggccacgggcaagagttacgcccgcgaggccggattcctgtacgaggcgggcgagttcgacgccgacttcttcgggatctcgccgcgcgaggccctcgccatggacccgcagcagcgtctcctcctggaggcctcctgggaggcgttcgagcacgccgggatcccggcggccaccgcgcgcggcacctcggtcggcgtcttcaccggcgtgatgtaccacgactacgccacccgtctcaccgatgtcccggagggcatcgagggctacctgggcaccggcaactccggcagtgtcgcctcgggccgcgtcgcgtacacgcttggcctggaggggccggccgtcacggtcgacaccgcctgctcgtcctcgctggtcgccctgcacctcgccgtgcaggccctgcgcaagggcgaggtcgacatggcgctcgccggcggcgtgacggtcatgtcgacgcccagcaccttcgtcgagttcagccgtcagcgcgggctggcgccggacggccggtcgaagtccttctcgtcgacggccgacggcaccagctggtccgagggcgtcggcgtcctcctcgtcgagcgcctgtccgacgcgcgtcgcaagggccatcggatcctcgccgtggtccggggcaccgccgtcaaccaggacggcgccagcagcggcctcacggctccgaacgggccgtcgcagcagcgcgtcatccgacgtgccctggcggacgcccggctcacgacctccgacgtggacgtcgtcgaggcccacggcacgggtacgcgactcggcgacccgatcgaggcgcaggccgtcatcgccacgtacgggcagggccgtgacggcgaacagccgctgcgcctcgggtcgttgaagtccaacatcggacacacccaggccgccgccggtgtctccggcgtgatcaagatggtccaggcgatgcgccacggcgtcctgccgaagacgctccacgtggagaagccgacggaccaggtggactggtccgcgggcgcggtcgagctgctcaccgaggccatggactggccggacaagggcgacggcggactgcgcagggccgcggtctcctccttcggcgtcagcgggacgaacgcgcacgtcgtgctcgaagaggccccggcggccgaggagacccctgcctccgaggcgaccccggccgtcgagccgtcggtcggcgccggcctggtgccgtggctggtgtcggcgaagactccggccgcgctggacgcccagatcggacgcctcgccgcgttcgcctcgcagggccgtacggacgccgccgatccgggcgcggtcgctcgcgtactggccggcgggcgcgccgagttcgagcaccgggccgtcgtgctcggcaccggacaggacgatttcgcgcaggcgctgaccgctccggaaggactgatacgcggcacgccctcggacgtgggccgggtggcgttcgtgttccccggtcagggcacgcagtgggccgggatgggcgccgaactcctcgacgtgtcgaaggagttcgcggcggccatggccgagtgcgagagcgcgctctcccgctatgtcgactggtcgctggaggccgtcgtccggcaggcgccgggcgcgcccacgctggagcgggtcgacgtcgtccagcccgtgaccttcgctgtcatggtttcgctggcgaaggtctggcagcaccacggcgtgacgccgcaggccgtcgtcggccactcgcagggcgagatcgccgccgcgtacgtcgccggtgccctcaccctcgacgacgccgcccgcgtcgtcaccctgcgcagcaagtccatcgccgcccacctcgccggcaagggcggcatgatctccctcgccctcagcgaggaagccacccggcagcgcatcgagaacctccacggactgtcgatcgccgccgtcaacggccccaccgccaccgtggtttcgggcgaccccacccagatccaagagctcgctcaggcgtgtgaggccgacggggtccgcgcacggatcatccccgtcgactacgcctcccacagcgcccacgtcgagaccatcgagagcgaactcgccgaggtcctcgccgggctcagcccgcggacacctgaggtgccgttcttctcgacactcgaaggcgcctggatcaccgagccggtgctcgacggcacctactggtaccgcaacctccgccaccgcgtcggcttcgcccccgccgtcgagaccctcgccaccgacgaaggcttcacccacttcatcgaggtcagcgcccaccccgtcctcaccatgaccctccccgagaccgtcaccggcctcggcaccctccgccgcgaacagggaggccaggagcgtctggtcacctcactcgccgaagcctggaccaacggcctcaccatcgactgggcgcccgtcctccccaccgcaaccggccaccaccccgagctccccacctacgccttccagcgccgtcactactggctccacgactcccccgccgtccagggctccgtgcaggactcctggcgctaccgcatcgactggaagcgcctcgcggtcgccgacgcgtccgagcgcgccgggctgtccgggcgctggctcgtcgtcgtccccgaggaccgttccgccgaggccgccccggtgctcgccgcgctgtccggcgccggcgccgaccccgtacagctggacgtgtccccgctgggcgaccggcagcggctcgccgcgacgctgggcgaggccctggcggcggccggtggagccgtcgacggcgtcctctcgctgctcgcgtgggacgagagcgcgcaccccggccaccccgcccccttcacccggggcaccggcgccaccctcaccctggtgcaggcgctggaggacgccggcgtcgccgccccgctgtggtgcgtgacccacggcgcggtgtccgtcggccgggccgaccacgtcacctcccccgcccaggccatggtgtggggcatgggccgggtcgccgccctggagcaccccgagcggtggggcggcctgatcgacctgccctcggacgccgaccgggcggccctggaccgcatgaccacggtcctcgccggcggtacgggtgaggaccaggtcgcggtacgcgcctccgggctgctcgcccgccgcctcgtccgcgcctccctcccggcgcacggcacggcttcgccgtggtggcaggccgacggcacggtgctcgtcaccggtgccgaggagcctgcggccgccgaggccgcacgccggctggcccgcgacggcgccggacacctcctcctccacaccaccccctccggcagcgaaggcgccgaaggcacctccggtgccgccgaggactccggcctcgccgggctcgtcgccgaactcgcggacctgggcgcgacggccaccgtcgtgacctgcgacctcacggacgcggaggcggccgcccggctgctcgccggcgtctccgacgcgcacccgctcagcgccgtcctccacctgccgcccaccgtcgactccgagccgctcgccgcgaccgacgcggacgcgctcgcccgtgtcgtgaccgcgaaggccaccgccgcgctccacctggaccgcctcctgcgggaggccgcggctgccggaggccgtccgcccgtcctggtcctcttctcctcggtcgccgcgatctggggcggcgccggtcagggcgcgtacgccgccggtacggccttcctcgacgccctcgccggtcagcaccgggccgacggccccaccgtgacctcggtggcctggagcccctgggagggcagccgcgtcaccgagggtgcgaccggggagcggctgcgccgcctcggcctgcgccccctcgcccccgcgacggcgctcaccgccctggacaccgcgctcggccacggcgacaccgccgtcacgatcgccgacgtcgactggtcgagcttcgcccccggcttcaccacggcccggccgggcaccctcctcgccgatctgcccgaggcgcgccgcgcgctcgacgagcagcagtcgacgacggccgccgacgacaccgtcctgagccgcgagctcggtgcgctcaccggcgccgaacagcagcgccgtatgcaggagttggtccgcgagcacctcgccgtggtcctcaaccacccctcccccgaggccgtcgacacggggcgggccttccgtgacctcggattcgactcgctgacggcggtcgagctccgcaaccgcctcaagaacgccaccggcctggccctcccggccactctggtcttcgactacccgaccccccggacgctggcggagttcctcctcgcggagatcctgggcgagcaggccggtgccggcgagcagcttccggtggacggcggggtcgacgacgagcccgtcgcgatcgtcggcatggcgtgccgcctgccgggcggtgtcgcctcgccggaggacctgtggcggctggtggccggcggcgaggacgcgatctccggcttcccgcaggaccgcggctgggacgtggaggggctgtacgacccggacccggacgcgtccgggcggacgtactgccgtgccggtggcttcctcgacgaggcgggcgagttcgacgccgacttcttcgggatctcgccgcgcgaggccctcgccatggacccgcagcagcggctcctcctggagacctcctgggaggccgtcgaggacgccgggatcgacccgacctcccttcaggggcagcaggtcggcgtgttcgcgggcaccaacggcccccactacgagccgctgctccgcaacaccgccgaggatcttgagggttacgtcgggacgggcaacgccgccagcatcatgtcgggccgtgtctcgtacaccctcggcctggagggcccggccgtcacggtcgacaccgcctgctcctcctcgctggtcgccctgcacctcgccgtgcaggccctgcgcaagggcgaatgcggactggcgctcgcgggcggtgtgacggtcatgtcgacgcccacgacgttcgtggagttcagccggcagcgcgggctcgcggaggacggccggtcgaaggcgttcgccgcgtcggcggacggcttcggcccggcggagggcgtcggcatgctcctcgtcgagcgcctgtcggacgcccgccgcaacggacaccgtgtgctggcggtcgtgcgcggcagcgcggtcaaccaggacggcgcgagcaacggcctgaccgccccgaacgggccctcgcagcagcgcgtcatccggcgcgcgctcgcggacgcccgactgacgaccgccgacgtggacgtcgtcgaggcccacggcacgggcacgcgactcggcgacccgatcgaggcacaggccctcatcgccacctacggccaggggcgcgacaccgaacagccgctgcgcctggggtcgttgaagtccaacatcggacacacccaggccgccgccggtgtctccggcatcatcaagatggtccaggcgatgcgccacggcgtcctgccgaagacgctccacgtggaccggccgtcggaccagatcgactggtcggcgggcacggtcgagctgctcaccgaggccatggactggccgaggaagcaggagggcgggctgcgccgcgcggccgtctcctccttcggcatcagcggcacgaacgcgcacatcgtgctcgaagaagccccggtcgacgaggacgccccggcggacgagccgtcggtcggcggtgtggtgccgtggctcgtgtccgcgaagactccggccgcgctggacgcccagatcggacgcctcgccgcgttcgcctcgcagggccgtacggacgccgccgatccgggcgcggtcgctcgcgtactggccggcgggcgtgcgcagttcgagcaccgggccgtcgcgctcggcaccggacaggacgacctggcggccgcactggccgcgcctgagggtctggtccggggtgtggcctccggtgtgggtcgagtggcgttcgtgttcccgggacagggcacgcagtgggccgggatgggtgccgaactcctcgacgtgtcgaaggagttcgcggcggccatggccgagtgcgaggccgcgctcgctccgtacgtggactggtcgctggaggccgtcgtccgacaggcccccggcgcgcccacgctggagcgggtcgatgtcgtccagcccgtgacgttcgccgtcatggtctcgctggcgaaggtctggcagcaccacggggtgaccccgcaagccgtcgtcggccactcgcagggcgagatcgccgccgcgtacgtcgccggtgccctgagcctggacgacgccgctcgtgtcgtgaccctgcgcagcaagtccatcggcgcccacctcgcgggccagggcggcatgctgtccctcgcgctgagcgaggcggccgttgtggagcgactggccgggttcgacgggctgtccgtcgccgccgtcaacgggcctaccgccaccgtggtttcgggcgacccgacccagatccaagagctcgctcaggcgtgtgaggccgacggggtccgcgcacggatcatccccgtcgactacgcctcccacagcgcccacgtcgagaccatcgagagcgaactcgccgacgtcctggcggggttgtccccccagacaccccaggtccccttcttctccaccctcgaaggcgcctggatcaccgaacccgccctcgacggcggctactggtaccgcaacctccgccatcgtgtgggcttcgccccggccgtcgaaaccctggccaccgacgaaggcttcacccacttcgtcgaggtcagcgcccaccccgtcctcaccatggcgctgcccgagaccgtcaccggactcggcaccctccgccgtgacaacggcggacagcaccgcctcaccacctccctcgccgaggcctgggccaacggcctcaccgtcgactgggcctctctcctccccaccacgaccacccaccccgatctgcccacctacgccttccagaccgagcgctactggccgcagcccgacctctccgccgccggtgacatcacctccgccggtctcggggcggccgagcacccgctgctcggcgcggccgtggcgctcgcggactccgacggctgcctgctcacggggagcctctccctccgtacgcacccctggctggcggaccacgcggtggccggcaccgtgctgctgccgggaacggcgttcgtggagctggcgttccgagccggggaccaggtcggttgcgatctggtcgaggagctcaccctcgacgcgccgctcgtgctgccccgtcgtggcgcggtccgtgtgcagctgtccgtcggcgcgagcgacgagtccgggcgtcgtaccttcgggctctacgcgcacccggaggacgcgccgggcgaggcggagtggacgcggcacgccaccggtgtgctggccgcccgtgcggaccgcaccgcccccgtcgccgacccggaggcctggccgccgccgggcgccgagccggtggacgtggacggtctgtacgagcgcttcgcggcgaacggctacggctacggccccctcttccagggcgtccgtggtgtctggcggcgtggcgacgaggtgttcgccgacgtggccctgccggccgaggtcgccggtgccgagggcgcgcggttcggccttcacccggcgctgctcgacgccgccgtgcaggcggccggtgcgggccggggcgttcggcgcgggcacgcggctgccgttcgcctggagcgggatctcctgtacgcggtcggcgccaccgccctccgcgtgcggctggcccccgccggcccggacacggtgtccgtgagcgccgccgactcctccgggcagccggtgttcgccgcggactccctcacggtgctgcccgtcgaccccgcgcagctggcggccttcagcgacccgactctggacgcgctgcacctgctggagtggaccgcctgggacggtgccgcgcaggccctgcccggcgcggtcgtgctgggcggcgacgccgacggtctcgccgcggcgctgcgcgccggtggcaccgaggtcctgtccttcccggaccttacggacctggtggaggccgtcgaccggggcgagaccccggccccggcgaccgtcctggtggcctgccccgccgccggccccgatgggccggagcatgtccgcgaggccctgcacgggtcgctcgcgctgatgcaggcctggctggccgacgagcggttcaccgatgggcgcctggtgctcgtgacccgcgacgcggtcgccgcccgttccggcgacggcctgcggtccacgggacaggccgccgtctggggcctcggccggtccgcgcagacggagagcccgggccggttcgtcctgctcgacctcgccggggaagcccggacggccggggacgccaccgccggggacggcctgacgaccggggacgccaccgtcggcggcacctctggagacgccgccctcggcagcgccctcgcgaccgccctcggctcgggcgagccgcagctcgccctccgggacggggcgctcctcgtaccccgcctggcgcgggccgccgcgcccgccgcggccgacggcctcgccgcggccgacggcctcgccgctctgccgctgcccgccgctccggccctctggcgtctggagcccggtacggacggcagcctggagagcctcacggcggcgcccggcgacgccgagaccctcgccccggagccgctcggcccgggacaggtccgcatcgcgatccgggccaccggtctcaacttccgcgacgtcctgatcgccctcggcatgtaccccgatccggcgctgatgggcaccgagggagccggcgtggtcaccgcgaccggccccggcgtcacgcacctcgcccccggcgaccgggtcatgggcctgctctccggcgcgtacgccccggtcgtcgtggcggacgcgcggaccgtcgcgcggatgcccgaggggtggacgttcgcccagggcgcctccgtgccggtggtgttcctgacggccgtctacgccctgcgcgacctggcggacgtcaagcccggcgagcgcctcctggtccactccgccgccggtggcgtgggcatggccgccgtgcagctcgcccggcactggggcgtggaggtccacggcacggcgagtcacgggaagtgggacgccctgcgcgcgctcggcctggacgacgcgcacatcgcctcctcccgcaccctggacttcgagtccgcgttccgtgccgcttccggcggggcgggcatggacgtcgtactgaactcgctcgcccgcgagttcgtcgacgcctcgctgcgcctgctcgggccgggcggccggttcgtggagatggggaagaccgacgtccgcgacgcggagcgggtcgccgccgaccaccccggtgtcggctaccgcgccttcgacctgggcgaggccgggccggagcggatcggcgagatgctcgccgaggtcatcgccctcttcgaggacggggtgctccggcacctgcccgtcacgacctgggacgtgcgccgggcccgcgacgccttccggcacgtcagccaggcccgccacacgggcaaggtcgtcctcacgatgccgtcgggcctcgacccggagggtacggtcctgctgaccggcggcaccggtgcgctggggggcatcgtggcccggcacgtggtgggcgagtggggcgtacgacgcctgctgctcgtgagccggcggggcacggacgccccgggcgccggcgagctcgtgcacgagctggaggccctgggagccgacgtctcggtggccgcgtgcgacgtcgccgaccgcgaagccctcaccgccgtactcgactcgatccccgccgaacacccgctcaccgcggtcgtccacacggcaggcgtcctctccgacggcaccctcccctcgatgacagcggaggatgtggaacacgtactgcgtcccaaggtcgacgccgcgttcctcctcgacgaactcacctcgacgcccggctacgacctggcagcgttcgtcatgttctcctccgccgccgccgtcttcggtggcgcggggcagggcgcctacgccgccgccaacgccaccctcgacgccctcgcctggcgccgccggacagccggactccccgccctctccctcggctggggcctctgggccgagaccagcggcatgaccggcggactcagcgacaccgaccgctcgcggctggcccgttccggggcgacgcccatggacagcgagctgaccctgtccctcctggacgcggccatgcgccgcgacgacccggcgctcgtcccgatcgccctggacgtcgccgcgctccgcgcccagcagcgcgacggcatgctggcgccgctgctcagcgggctcacccgcggatcgcgggtcggcggcgcgccggtcaaccagcgcagggcagccgccggaggcgcgggcgaggcggacacggacctcggcgggcggctcgccgcgatgacaccggacgaccgggtcgcgcacctgcgggacctcgtccgtacgcacgtggcgaccgtcctgggacacggcaccccgagccgggtggacctggagcgggccttccgcgacaccggtttcgactcgctcaccgccgtcgaactccgcaaccgtctcaacgccgcgaccgggctgcggctgccggccacgctggtcttcgaccaccccaccccgggggagctcgccgggcacctgctcgacgaactcgccacggccgcgggcgggtcctgggcggaaggcaccgggtccggagacacggcctcggcgaccgatcggcagaccacggcggccctcgccgaactcgaccggctggaaggcgtgctcgcctccctcgcgcccgccgccggcggccgtccggagctcgccgcccggctcagggcgctggccgcggccctgggggacgacggcgacgacgccaccgacctggacgaggcgtccgacgacgacctcttctccttcatcgacaaggagctgggcgactccgacttctgacctgcccgacaccaccggcaccaccggcaccaccagcccccctcacacacggaacacggaacggacaggcgagaacgggagccatggcgaacaacgaagacaagctccgcgactacctcaagcgcgtcaccgccgagctgcagcagaacaccaggcgtctgcgcgagatcgagggacgcacgcacgagccggtggcgatcgtgggcatggcctgccgcctgccgggcggtgtcgcctcgcccgaggacctgtggcagctggtggccggggacggggacgcgatctcggagttcccgcaggaccgcggctgggacgtggaggggctgtacgaccccgacccggacgcgtccggcaggacgtactgccggtccggcggattcctgcacgacgccggcgagttcgacgccgacttcttcgggatctcgccgcgcgaggccctcgccatggacccgcagcagcgactgtccctcaccaccgcgtgggaggcgatcgagagcgcgggcatcgacccgacggccctgaagggcagcggcctcggcgtcttcgtcggcggctggcacaccggctacacctcggggcagaccaccgccgtgcagtcgcccgagctggagggccacctggtcagcggcgcggcgctgggcttcctgtccggccgtatcgcgtacgtcctcggtacggacggaccggccctgaccgtggacacggcctgctcgtcctcgctggtcgccctgcacctcgccgtgcaggccctccgcaagggcgagtgcgacatggccctcgccggtggtgtcacggtcatgcccaacgcggacctgttcgtgcagttcagccggcagcgcgggctggccgcggacggccggtcgaaggcgttcgccacctcggcggacggcttcggccccgcggagggcgccggagtcctgctggtggagcgcctgtcggacgcccgccgcaacggacaccggatcctcgcggtcgtccgcggcagcgcggtcaaccaggacggcgccagcaacggcctcacggctccgcacgggccctcccagcagcgcgtcatccgacgggccctggcggacgcccggctcgcgccgggtgacgtggacgtcgtcgaggcgcacggcacgggcacgcggctcggcgacccgatcgaggcgcaggccctcatcgccacctacggccaggagaagagcagcgaacagccgctgaggctgggcgcgttgaagtcgaacatcgggcacacgcaggccgcggccggtgtcgcaggtgtcatcaagatggtccaggcgatgcgccacggactgctgccgaagacgctgcacgtcgacgagccctcggaccagatcgactggtcggcgggcacggtggaactcctcaccgaggccgtcgactggccggagaagcaggacggcgggctgcgccgcgcggctgtctcctccttcggcatcagcgggacgaacgcgcacgtcgtcctggaggaggccccggcggtcgaggactccccggccgtcgagccgccggccggtggcggtgtggtgccgtggccggtgtccgcgaagactccggccgcgctggacgcccagatcgggcagctcgccgcgtacgcggacggtcgtacggacgtggatccggcggtggccgcccgcgccctggtcgacagccgtacggcgatggagcaccgcgcggtcgcggtcggcgacagccgggaggcactgcgggacgccctgcggatgccggaaggactggtacgcggcacgtcctcggacgtgggccgggtggcgttcgtcttccccggccagggcacgcagtgggccggcatgggcgccgaactccttgacagctcaccggagttcgctgcctcgatggccgaatgcgagaccgcgctctcccgctacgtcgactggtctcttgaagccgtcgtccgacaggaacccggcgcacccacgctcgaccgcgtcgacgtcgtccagcccgtgaccttcgctgtcatggtctcgctggcgaaggtctggcagcaccacggcatcaccccccaggccgtcgtcggccactcgcagggcgagatcgccgccgcgtacgtcgccggtgcactcaccctcgacgacgccgcccgcgtcgtcaccctgcgcagcaagtccatcgccgcccacctcgccggcaagggcggcatgatctccctcgccctcgacgaggcggccgtcctgaagcgactgagcgacttcgacggactctccgtcgccgccgtcaacggccccaccgccaccgtcgtctccggcgacccgacccagatcgaggaactcgcccgcacctgcgaggccgacggcgtccgtgcgcggatcatcccggtcgactacgcctcccacagccggcaggtcgagatcatcgagaaggagctggccgaggtcctcgccggactcgccccgcaggctccgcacgtgccgttcttctccaccctcgaaggcacctggatcaccgagccggtgctcgacggcacctactggtaccgcaacctgcgccatcgcgtgggcttcgcccccgccgtggagaccttggcggttgacggcttcacccacttcatcgaggtcagcgcccaccccgtcctcaccatgaccctccccgagaccgtcaccggcctcggcaccctccgccgcgaacagggaggccaggagcgtctggtcacctcactcgccgaagcctgggccaacggcctcaccatcgactgggcgcccatcctccccaccgcaaccggccaccaccccgagctccccacctacgccttccagaccgagcgcttctggctgcagagctccgcgcccaccagcgccgccgacgactggcgttaccgcgtcgagtggaagccgctgacggcctccggccaggcggacctgtccgggcggtggatcgtcgccgtcgggagcgagccagaagccgagctgctgggcgcgctgaaggccgcgggagcggaggtcgacgtactggaagccggggcggacgacgaccgtgaggccctcgccgcccggctcaccgcactgacgaccggcgacggcttcaccggcgtggtctcgctcctcgacgacctcgtgccacaggtcgcctgggtgcaggcactcggcgacgccggaatcaaggcgcccctgtggtccgtcacccagggcgcggtctccgtcggacgtctcgacacccccgccgaccccgaccgggccatgctctggggcctcggccgcgtcgtcgcccttgagcaccccgaacgctgggccggcctcgtcgacctccccgcccagcccgatgccgccgccctcgcccacctcgtcaccgcactctccggcgccaccggcgaggaccagatcgccatccgcaccaccggactccacgcccgccgcctcgcccgcgcacccctccacggacgtcggcccacccgcgactggcagccccacggcaccgtcctcatcaccggcggcaccggagccctcggcagccacgccgcacgctggatggcccaccacggagccgaacacctcctcctcgtcagccgcagcggcgaacaagcccccggagccacccaactcaccgccgaactcaccgcatcgggcgcccgcgtcaccatcgccgcctgcgacgtcgccgacccccacgccatgcgcaccctcctcgacgccatccccgccgagacgcccctcaccgccgtcgtccacaccgccggcgcaccgggcggcgatccgctggacgtcaccggcccggaggacatcgcccgcatcctgggcgcgaagacgagcggcgccgaggtcctcgacgacctgctccgcggcactccgctggacgccttcgtcctctactcctcgaacgccggggtctggggcagcggcagccagggcgtctacgcggcggccaacgcccacctcgacgcgctcgccgcccggcgccgcgcccggggcgagacggcgacctcggtcgcctggggcctctgggccggcgacggcatgggccggggcgccgacgacgcgtactggcagcgtcgcggcatccgtccgatgagccccgaccgcgccctggacgaactggccaaggccctgagccacgacgagaccttcgtcgccgtggccgatgtcgactgggagcggttcgcgcccgcgttcacggtgtcccgtcccagccttctgctcgacggcgtcccggaggcccggcaggcgctcgccgcacccgtcggtgccccggctcccggcgacgccgccgtggcgccgaccgggcagtcgtcggcgctggccgcgatcaccgcgctccccgagcccgagcgccggccggcgctcctcaccctcgtccgtacccacgcggcggccgtactcggccattcctcccccgaccgggtggcccccggccgtgccttcaccgagctcggcttcgactcgctgacggccgtgcagctccgcaaccagctctccacggtggtcggcaacaggctccccgccaccacggtcttcgaccacccgacgcccgccgcactcgccgcgcacctccacgaggcgtacctcgcaccggccgagccggccccgacggactgggaggggcgggtgcgccgggccctggccgaactgcccctcgaccggctgcgggacgcgggggtcctcgacaccgtcctgcgcctcaccggcatcgagcccgagccgggttccggcggttcggacggcggcgccgccgaccctggtgcggagccggaggcgtcgatcgacgacctggacgccgaggccctgatccggatggctctcggcccccgtaacacctgacccgaccgcggtcctgccccacgcgccgcaccccgcgcatcccgcgcaccacccgcccccacacgcccacaaccccatccacgagcggaagaccacacccagatgacgagttccaacgaacagttggtggacgctctgcgcgcctctctcaaggagaacgaagaactccggaaagagagccgtcgccgggccgaccgtcggcaggagcccatggcgatcgtcggcatgagctgccggttcgcgggcggaatccggtcccccgaggacctctgggacgccgtcgccgcgggcaaggacctggtctccgaggtaccggaggagcgcggctgggacatcgactccctctacgacccggtgcccgggcgcaagggcacgacgtacgtccgcaacgccgcgttcctcgacgacgccgccggattcgacgcggccttcttcgggatctcgccgcgcgaggccctcgccatggacccgcagcagcggcagctcctcgaagcctcctgggaggtcttcgagcgggccggcatcgaccccgcgtcggtccgcggcaccgacgtcggcgtgtacgtgggctgtggctaccaggactacgcgccggacatccgggtcgcccccgaaggcaccggcggttacgtcgtcaccggcaactcctccgccgtggcctccgggcgcatcgcgtactccctcggcctggagggacccgccgtgaccgtggacacggcgtgctcctcttcgctcgtcgccctgcacctcgccctgaagggcctgcggaacggcgactgctcgacggcactcgtgggcggcgtggccgtcctcgcgacgccgggcgcgttcatcgagttcagcagccagcaggccatggccgccgacggccggaccaagggcttcgcctcggcggcggacggcctcgcctggggcgagggcgtcgccgtactcctcctcgaacggctctccgacgcgcggcgcaagggccaccgggtcctggccgtcgtgcgcggcagcgccatcaaccaggacggcgcgagcaacggcctcacggctccgcacgggccctcccagcagcacctgatccgccaggccctggccgacgcgcggctcacgtcgagcgacgtggacgtcgtggagggccacggcacggggacccgtctcggcgacccgatcgaggcgcaggcgctgctcgccacgtacgggcaggggcgcgccccggggcagccgctgcggctggggacgctgaagtcgaacatcgggcacacgcaggccgcttcgggtgtcgccggtgtcatcaagatggtgcaggcgctgcgccacggggtgctgccgaagaccctgcacgtggacgagccgacggaccaggtcgactggtcggccggttcggtcgagctgctcaccgaggccgtggactggccggagcggccgggccggctccgccgggcgggcgtctccgcgttcggcgtgggcgggacgaacgcgcacgtcgtcctggaggaggccccggcggtcgaggagtcccctgccgtcgagccgccggccggtggcggcgtggtgccgtggccggtgtccgcgaagacctcggccgcactggacgcccagatcgggcagctcgccgcatacgcggaagaccgcacggacgtggatccggcggtggccgcccgcgccctggtcgacagccgtacggcgatggagcaccgcgcggtcgcggtcggcgacagccgggaggcactgcgggacgccctgcggatgccggaaggactggtacggggcacggtcaccgatccgggccgggtggcgttcgtcttccccggccagggcacgcagtgggccggcatgggcgccgaactcctcgacagctcacccgaattcgccgccgccatggccgaatgcgagaccgcactctccccgtacgtcgactggtctctcgaagccgtcgtccgacaggctcccagcgcaccgacactcgaccgcgtcgacgtcgtccagcccgtcaccttcgccgtcatggtctccctcgccaaggtctggcagcaccacggcatcacccccgaggccgtcatcggccactcccagggcgagatcgccgccgcgtacgtcgccggtgccctcaccctcgacgacgccgctcgtgtcgtgaccctccgcagcaagtccatcgccgcccacctcgccggcaagggcggcatgatctccctcgccctcagcgaggaagccacccggcagcgcatcgagaacctccacggactgtcgatcgccgccgtcaacgggcctaccgccaccgtggtttcgggcgaccccacccagatccaagaacttgctcaggcgtgtgaggccgacggcatccgcgcacggatcatccccgtcgactacgcctcccacagcgcccacgtcgagaccatcgagaacgaactcgccgacgtcctggcggggttgtccccccagacaccccaggtccccttcttctccaccctcgaaggcacctggatcaccgaacccgccctcgacggcggctactggtaccgcaacctccgccatcgtgtgggcttcgccccggccgtcgagaccctcgccaccgacgaaggcttcacccacttcatcgaggtcagcgcccaccccgtcctcaccatgaccctccccgacaaggtcaccggcctggccaccctccgacgcgaggacggcggacagcaccgcctcaccacctcccttgccgaggcctgggccaacggcctcgccctcgactgggcctccctcctgcccgccacgggcgccctcagccccgccgtccccgacctcccgacgtacgccttccagcaccgctcgtactggatcagccccgcgggtcccggcgaggcgcccgcgcacaccgcttccgggcgcgaggccgtcgccgagacggggctcgcgtggggcccgggtgccgaggacctcgacgaggagggccggcgcagcgccgtactcgcgatggtgatgcggcaggcggcctccgtgctccggtgcgactcgcccgaagaggtccccgtcgaccgcccgctgcgggagatcggcttcgactcgctgaccgccgtcgacttccgcaaccgcgtcaaccggctgaccggtctccagctgccgcccaccgtcgtgttccagcacccgacgcccgtcgcgctcgccgagcgcatcagcgacgagctggccgagcggaactgggccgtcgccgagccgtcggatcacgagcaggcggaggaggagaaggccgccgctccggcgggggcccgctccggggccgacaccggcgccggcgccgggatgttccgcgccctgttccggcaggccgtggaggacgaccggtacggcgagttcctcgacgtcctcgccgaagcctccgcgttccgcccgcagttcgcctcgcccgaggcctgctcggagcggctcgacccggtgctgctcgccggcggtccgacggaccgggcggaaggccgtgccgttctcgtcggctgcaccggcaccgcggcgaacggcggcccgcacgagttcctgcggctcagcacctccttccaggaggagcgggacttcctcgccgtacctctccccggctacggcacgggtacgggcaccggcacggccctcctcccggccgatctcgacaccgcgctcgacgcccaggcccgggcgatcctccgggccgccggggacgccccggtcgtcctgctcgggcactccggcggcgccctgctcgcgcacgagctggccttccgcctggagcgggcgcacggcgcgccgccggccgggatcgtcctggtcgacccctatccgccgggccatcaggagcccatcgaggtgtggagcaggcagctgggcgagggcctgttcgcgggcgagctggagccgatgtccgatgcgcggctgctggccatgggccggtacgcgcggttcctcgccggcccgcggccgggccgcagcagcgcgcccgtgcttctggtccgtgcctccgaaccgctgggcgactggcaggaggagcggggcgactggcgtgcccactgggaccttccgcacaccgtcgcggacgtgccgggcgaccacttcacgatgatgcgggaccacgcgccggccgtcgccgaggccgtcctctcctggctcgacgccatcgagggcatcgagggggcgggcaagtgaccgacagacctctgaacgtggacagcggactgtggatccggcgcttccaccccgcgccgaacagcgcggtgcggctggtctgcctgccgcacgccggcggctccgccagctacttcttccgcttctcggaggagctgcacccctccgtcgaggccctgtcggtgcagtatccgggccgccaggaccggcgtgccgagccgtgtctggagagcgtcgaggagctcgccgagcatgtggtcgcggccaccgaaccctggtggcaggagggccggctggccttcttcgggcacagcctcggcgcctccgtcgccttcgagacggcccgcatcctggaacagcggcacggggtacggcccgagggcctgtacgtctccggtcggcgcgccccgtcgctggcgccggaccggctcgtccaccagctggacgaccgggcgttcctggccgagatccggcggctcagcggcaccgacgagcggttcctccaggacgacgagctgctgcggctggtgctgcccgcgctgcgcagcgactacaaggcggcggagacgtacctgcaccggccgtccgccaagctcacctgcccggtgatggccctggccggcgaccgtgacccgaaggcgccgctgaacgaggtggccgagtggcgtcggcacaccagcgggccgttctgcctccgggcgtactccggcggccacttctacctcaacgaccagtggcacgagatctgcaacgacatctccgaccacctgctcgtcacccgcggcgcgcccgatgcccgcgtcgtgcagcccccgaccagccttatcgaaggagcggcgaagagatggcagaacccacggtgaccgacgacctgacgggggccctcacgcagcccccgctgggccgcaccgtccgcgcggtggccgaccgtgaactcggcacccacctcctggagacccgcggcatccactggatcc

SEQ ID NO: 6 - protein, 12199 aa, Streptomyces venezuelae
MAFSPQGGRHELGQNFLVDRSVIDEIDGLVARTKGPILEIGPGDGALTLPLSRHGRPITAVELDGRRAQRLGARTPGHVTVVHHDFLQYPLPRNPHVVVGNVPFHLTTAIMRRLLDAQHWHTAVLLVQWEVARRRAGVGGSTLLTAGWAPWYEFDLHSRVPARAFRPMPGVDGGVLAIRRRSAPLVGQVKTYQDFVRQVFTGKGNGLKEILRRTGRISQRDLATWLRRNEISPHALPKDLKPGQWASLWELTGGTADGSFDGTAGGGAAGSHGAARVGAGHPGGRVSASRRGVPQARRGRGHAVRSSTGTEPRWGRGRAESAMAMRDSIPRRADRDTLRRELGQNFLQDDRAVRNLVTHVEGDGRNVLEIGPGKGAITEELVRSFDTVTVVEMDPHWAAHVRRKFEGERVTVFQGDFLDFRIPRDIDTVVGNVPFGITTQILRSLLESTNWQSAALIVQWEVARKRAGRSGGSLLTTSWAPWYEFAVHDRVRASSFRPMPRVDGGVLTIRRRPQPLLPESASRAFQNFAEAVFTGPGRGLAEILRRHIPKRTYRSLADRHGIPDGGLPKDLTLTQWIALFQASQPSYAPGAPGTRMPGQGGGAGGRDYDSETSRAAVPGSRRYGPTRGGEPCAPRAQVRQTKGRQGARGSSYGRRTGRMSSAGITRTGARTPVTGRGAAAWDTGEVRVRRGLPPAGPDHAEHSFSRAPTGDVRAELIRGEMSTVSKSESEEFVSVSNDAGSAHGTAEPVAVVGISCRVPGARDPREFWELLAAGGQAVTDVPADRWNAGDFYDPDRSAPGRSNSRWGGFIEDVDRFDAAFFGISPREAAEMDPQQRLALELGWEALERAGIDPSSLTGTRTGVFAGAIWDDYATLKHRQGGAAITPHTVTGLHRGIIANRLSYTLGLRGPSMVVDSGQSSSLVAVHLACESLRRGESELALAGGVSLNLVPDSIIGASKFGGLSPDGRAYTFDARANGYVRGEGGGFVVLKRLSRAVADGDPVLAVIRGSAVNNGGAAQGMTTPDAQAQEAVLREAHERAGTAPADVRYVELHGTGTPVGDPIEAAALGAALGTGRPAGQPLLVGSVKTNIGHLEGAAGIAGLIKAVLAVRGRALPASLNYETPNPAIPFEELNLRVNTEYLPWEPEHDGQRMVVGVSSFGMGGTNAHVVLEEAPGGCRGASVVESTVGGSAVGGGVVPWVVSAKSAAALDAQIERLAAFASRDRTDGVDAGAVDAGAVDAGAVARVLAGGRAQFEHRAVVVGSGPDDLAAALAAPEGLVRGVASGVGRVAFVFPGQGTQWAGMGAELLDSSAVFAAAMAECEAALSPYVDWSLEAVVRQAPGAPTLERVDVVQPVTFAVMVSLARVWQHHGVTPQAVVGHSQGEIAAAYVAGALSLDDAARVVTLRSKSIAAHLAGKGGMLSLALSEDAVLERLAGFDGLSVAAVNGPTATVVSGDPVQIEELARACEADGVRARVIPVDYASHSRQVEIIESELAEVLAGLSPQAPRVPFFSTLEGAWITEPVLDGGYWYRNLRHRVGFAPAVETLATDEGFTHFVEVSAHPVLTMALPGTVTGLATLRRDNGGQDRLVASLAEAWANGLAVDWSPLLPSATGHHSDLPTYAFQTERHWLGEIEALAPAGEPAVQPAVLRTEAAEPAELDRDEQLRVILDKVRAQTAQVLGYATGGQIEVDRTFREAGCTSLTGVDLRNRINAAFGVRMAPSMIFDFPTPEALAEQLLLVVHGEAAANPAGAEPAPVAAAGAVDEPVAIVGMACRLPGGVASPEDLWRLVAGGGDAISEFPQDRGWDVEGLYHPDPEHPGTSYVRQGGFIENVAGFDAAFFGISPREALAMDPQQRLLLETSWEAVEDAGIDPTSLRGRQVGVFTGAMTHEYGPSLRDGGEGLDGYLLTGNTASVMSGRVSYTLGLEGPALTVDTACSSSLVALHLAVQALRKGEVDMALAGGVAVMPTPGMFVEFSRQRGLAGDGRSKAFAASADGTSWSEGVGVLLVERLSDARRNGHQVLAVVRGSALNQDGASNGLTAPNGPSQQRVIRRALADARLTTSDVDVVEAHGTGTRLGDPIEAQALIATYGQGRDDEQPLRLGSLKSNIGHTQAAAGVSGVIKMVQAMRHGLLPKTLHVDEPSDQIDWSAGAVELLTEAVDWPEKQDGGLRRAAVSSFGISGTNAHVVLEEAPVVVEGASVVEPSVGGSAVGGGVTPWVVSAKSAAALDAQIERLAAFASRDRTDDADAGAVDAGAVAHVLADGRAQFEHRAVALGAGADDLVQALADPDGLIRGTASGVGRVAFVFPGQGTQWAGMGAELLDSSAVFAAAMAECEAALSPYVDWSLEAVVRQAPGAPTLERVDVVQPVTFAVMVSLARVWQHHGVTPQAVVGHSQGEIAAAYVAGALPLDDAARVVTLRSKSIAAHLAGKGGMLSLALNEDAVLERLSDFDGLSVAAVNGPTATVVSGDPVQIEELAQACKADGFRARIIPVDYASHSRQVEIIESELAQVLAGLSPQAPRVPFFSTLEGTWITEPVLDGTYWYRNLRHRVGFAPAIETLAVDEGFTHFVEVSAHPVLTMTLPETVTGLGTLRREQGGQERLVTSLAEAWVNGLPVAWTSLLPATASRPGLPTYAFQAERYWLENTPAALATGDDWRYRIDWKRLPAAEGSERTGLSGRWLAVTPEDHSAQAAAVLTALVDAGAKVEVLTAGADDDREALAARLTALTTGDGFTGVVSLLDGLVPQVAWVQALGDAGIKAPLWSVTQGAVSVGRLDTPADPDRAMLWGLGRVVALEHPERWAGLVDLPAQPDAAALAHLVTALSGATGEDQIAIRTTGLHARRLARAPLHGRRPTRDWQPHGTVLITGGTGALGSHAARWMAHHGAEHLLLVSRSGEQAPGATQLTAELTASGARVTIAACDVADPHAMRTLLDAIPAETPLTAVVHTAGALDDGIVDTLTAEQVRRAHRAKAVGASVLDELTRDLDLDAFVLFSSVSSTLGIPGQGNYAPHNAYLDALAARRRATGRSAVSVAWGPWDGGGMAAGDGVAERLRNHGVPGMDPELALAALESALGRDETAITVADIDWDRFYLAYSSGRPQPLVEELPEVRRIIDARDSATSGQGGSSAQGANPLAERLAAAAPGERTEILLGLVRAQAAAVLRMRSPEDVAADRAFKDIGFDSLAGVELRNRLTRATGLQLPATLVFDHPTPLALVSLLRSEFLGDEETADARRSAALPATVGAGAGAGAGTDADDDPIAIVAMSCRYPGDIRSPEDLWRMLSEGGEGITPFPTDRGWDLDGLYDADPDALGRAYVREGGFLHDAAEFDAEFFGVSPREALAMDPQQRMLLTTSWEAFERAGIEPASLRGSSTGVFIGLSYQDYAARVPNAPRGVEGYLLTGSTPSVASGRIAYTFGLEGPATTVDTACSSSLTALHLAVRALRSGECTMALAGGVAMMATPHMFVEFSRQRALAPDGRSKAFSADADGFGAAEGVGLLLVERLSDARRNGHPVLAVVRGTAVNQDGASNGLTAPNGPSQQRVIRQALADARLAPGDIDAVETHGTGTSLGDPIEAQGLQATYGKERPAERPLAIGSVKSNIGHTQAAAGAAGIIKMVLAMRHGTLPKTLHADEPSPHVDWANSGLALVTEPIDWPAGTGPRRAAVSSFGISGTNAHVVLEQAPDAAGEVLGADEVPEVSETVAMAGTAGTSEVAEGSEASEAPAAPGSREASLPGHLPWVLSAKDEQSLRGQAAALHAWLSEPAADLSDADGPARLRDVGYTLATSRTAFAHRAAVTAADRDGFLDGLATLAQGGTSAHVHLDTARDGTTAFLFTGQGSQRPGAGRELYDRHPVFARALDEICAHLDGHLELPLLDVMFAAEGSAEAALLDETRYTQCALFALEVALFRLVESWGMRPAALLGHSVGEIAAAHVAGVFSLADAARLVAARGRLMQELPAGGAMLAVQAAEDEIRVWLETEERYAGRLDVAAVNGPEAAVLSGDADAAREAEAYWSGLGRRTRALRVSHAFHSAHMDGMLDGFRAVLETVEFRRPSLTVVSNVTGLAAGPDDLCDPEYWVRHVRGTVRFLDGVRVLRDLGVRTCLELGPDGVLTAMAADGLADTPADSAAGSPVGSPAGSPADSAAGALRPRPLLVALLRRKRSETETVADALGRAHAHGTGPDWHAWFAGSGAHRVDLPTYSFRRDRYWLDAPAADTAVDTAGLGLGTADHPLLGAVVSLPDRDGLLLTGRLSLRTHPWLADHAVLGSVLLPGAAMVELAAHAAESAGLRDVRELTLLEPLVLPEHGGVELRVTVGAPAGEPGGESAGDGARPVSLHSRLADAPAGTAWSCHATGLLATDRPELPVAPDRAAMWPPQGAEEVPLDGLYERLDGNGLAFGPLFQGLNAVWRYEGEVFADIALPATTNATAPATANGGGSAAAAPYGIHPALLDASLHAIAVGGLVDEPELVRVPFHWSGVTVHAAGAAAARVRLASAGTDAVSLSLTDGEGRPLVSVERLTLRPVTADQAAASRVGGLMHRVAWRPYALASSGEQDPHATSYGPTAVLGKDELKVAAALESAGVEVGLYPDLAALSQDVAAGAPAPRTVLAPLPAGPADGGAEGVRGTVARTLELLQAWLADEHLAGTRLLLVTRGAVRDPEGSGADDGGEDLSHAAAWGLVRTAQTENPGRFGLLDLADDASSYRTLPSVLSDAGLRDEPQLALHDGTIRLARLASVRPETGTAAPALAPEGTVLLTGGTGGLGGLVARHVVGEWGVRRLLLVSRRGTDAPGADELVHELEALGADVSVAACDVADREALTAVLDAIPAEHPLTAVVHTAGVLSDGTLPSMTTEDVEHVLRPKVDAAFLLDELTSTPAYDLAAFVMFSSAAAVFGGAGQGAYAAANATLDALAWRRRAAGLPALSLGWGLWAETSGMTGELGQADLRRMSRAGIGGISDAEGIALLDAALRDDRHPVLLPLRLDAAGLRDAAGNDPAGIPALFRDVVGARTVRARPSAASASTTAGTAGTPGTADGAAETAAVTLADRAATVDGPARQRLLLEFVVGEVAEVLGHARGHRIDAERGFLDLGFDSLTAVELRNRLNSAGGLALPATLVFDHPSPAALASHLDAELPRGASDQDGAGNRNGNENGTTASRSTAETDALLAQLTRLEGALVLTGLSDAPGSEEVLEHLRSLRSMVTGETGTGTASGAPDGAGSGAEDRPWAAGDGAGGGSEDGAGVPDFMNASAEELFGLLDQDPSTDMSTVNEEKYLDYLRRATADLHEARGRLRELEAKAGEPVAIVGMACRLPGGVASPEDLWRLVAGGEDAISEFPQDRGWDVEGLYDPNPEATGKSYAREAGFLYEAGEFDADFFGISPREALAMDPQQRLLLEASWEAFEHAGIPAATARGTSVGVFTGVMYHDYATRLTDVPEGIEGYLGTGNSGSVASGRVAYTLGLEGPAVTVDTACSSSLVALHLAVQALRKGEVDMALAGGVTVMSTPSTFVEFSRQRGLAPDGRSKSFSSTADGTSWSEGVGVLLVERLSDARRKGHRILAVVRGTAVNQDGASSGLTAPNGPSQQRVIRRALADARLTTSDVDVVEAHGTGTRLGDPIEAQAVIATYGQGRDGEQPLRLGSLKSNIGHTQAAAGVSGVIKMVQAMRHGVLPKTLHVEKPTDQVDWSAGAVELLTEAMDWPDKGDGGLRRAAVSSFGVSGTNAHVVLEEAPAAEETPASEATPAVEPSVGAGLVPWLVSAKTPAALDAQIGRLAAFASQGRTDAADPGAVARVLAGGRAEFEHRAVVLGTGQDDFAQALTAPEGLIRGTPSDVGRVAFVFPGQGTQWAGMGAELLDVSKEFAAAMAECESALSRYVDWSLEAVVRQAPGAPTLERVDVVQPVTFAVMVSLAKVWQHHGVTPQAVVGHSQGEIAAAYVAGALTLDDAARVVTLRSKSIAAHLAGKGGMISLALSEEATRQRIENLHGLSIAAVNGPTATVVSGDPTQIQELAQACEADGVRARIIPVDYASHSAHVETIESELAEVLAGLSPRTPEVPFFSTLEGAWITEPVLDGTYWYRNLRHRVGFAPAVETLATDEGFTHFIEVSAHPVLTMTLPETVTGLGTLRREQGGQERLVTSLAEAWTNGLTIDWAPVLPTATGHHPELPTYAFQRRHYWLHDSPAVQGSVQDSWRYRIDWKRLAVADASERAGLSGRWLVVVPEDRSAEAAPVLAALSGAGADPVQLDVSPLGDRQRLAATLGEALAAAGGAVDGVLSLLAWDESAHPGHPAPFTRGTGATLTLVQALEDAGVAAPLWCVTHGAVSVGRADHVTSPAQAMVWGMGRVAALEHPERWGGLIDLPSDADRAALDRMTTVLAGGTGEDQVAVRASGLLARRLVRASLPAHGTASPWWQADGTVLVTGAEEPAAAEAARRLARDGAGHLLLHTTPSGSEGAEGTSGAAEDSGLAGLVAELADLGATATVVTCDLTDAEAAARLLAGVSDAHPLSAVLHLPPTVDSEPLAATDADALARVVTAKATAALHLDRLLREAAAAGGRPPVLVLFSSVAAIWGGAGQGAYAAGTAFLDALAGQHRADGPTVTSVAWSPWEGSRVTEGATGERLRRLGLRPLAPATALTALDTALGHGDTAVTIADVDWSSFAPGFTTARPGTLLADLPEARRALDEQQSTTAADDTVLSRELGALTGAEQQRRMQELVREHLAVVLNHPSPEAVDTGRAFRDLGFDSLTAVELRNRLKNATGLALPATLVFDYPTPRTLAEFLLAEILGEQAGAGEQLPVDGGVDDEPVAIVGMACRLPGGVASPEDLWRLVAGGEDAISGFPQDRGWDVEGLYDPDPDASGRTYCRAGGFLDEAGEFDADFFGISPREALAMDPQQRLLLETSWEAVEDAGIDPTSLQGQQVGVFAGTNGPHYEPLLRNTAEDLEGYVGTGNAASIMSGRVSYTLGLEGPAVTVDTACSSSLVALHLAVQALRKGECGLALAGGVTVMSTPTTFVEFSRQRGLAEDGRSKAFAASADGFGPAEGVGMLLVERLSDARRNGHRVLAVVRGSAVNQDGASNGLTAPNGPSQQRVIRRALADARLTTADVDVVEAHGTGTRLGDPIEAQALIATYGQGRDTEQPLRLGSLKSNIGHTQAAAGVSGIIKMVQAMRHGVLPKTLHVDRPSDQIDWSAGTVELLTEAMDWPRKQEGGLRRAAVSSFGISGTNAHIVLEEAPVDEDAPADEPSVGGVVPWLVSAKTPAALDAQIGRLAAFASQGRTDAADPGAVARVLAGGRAQFEHRAVALGTGQDDLAAALAAPEGLVRGVASGVGRVAFVFPGQGTQWAGMGAELLDVSKEFAAAMAECEAALAPYVDWSLEAVVRQAPGAPTLERVDVVQPVTFAVMVSLAKVWQHHGVTPQAVVGHSQGEIAAAYVAGALSLDDAARVVTLRSKSIGAHLAGQGGMLSLALSEAAVVERLAGFDGLSVAAVNGPTATVVSGDPTQIQELAQACEADGVRARIIPVDYASHSAHVETIESELADVLAGLSPQTPQVPFFSTLEGAWITEPALDGGYWYRNLRHRVGFAPAVETLATDEGFTHFVEVSAHPVLTMALPETVTGLGTLRRDNGGQHRLTTSLAEAWANGLTVDWASLLPTTTTHPDLPTYAFQTERYWPQPDLSAAGDITSAGLGAAEHPLLGAAVALADSDGCLLTGSLSLRTHPWLADHAVAGTVLLPGTAFVELAFRAGDQVGCDLVEELTLDAPLVLPRRGAVRVQLSVGASDESGRRTFGLYAHPEDAPGEAEWTRHATGVLAARADRTAPVADPEAWPPPGAEPVDVDGLYERFAANGYGYGPLFQGVRGVWRRGDEVFADVALPAEVAGAEGARFGLHPALLDAAVQAAGAGRGVRRGHAAAVRLERDLLYAVGATALRVRLAPAGPDTVSVSAADSSGQPVFAADSLTVLPVDPAQLAAFSDPTLDALHLLEWTAWDGAAQALPGAVVLGGDADGLAAALRAGGTEVLSFPDLTDLVEAVDRGETPAPATVLVACPAAGPDGPEHVREALHGSLALMQAWLADERFTDGRLVLVTRDAVAARSGDGLRSTGQAAVWGLGRSAQTESPGRFVLLDLAGEARTAGDATAGDGLTTGDATVGGTSGDAALGSALATALGSGEPQLALRDGALLVPRLARAAAPAAADGLAAADGLAALPLPAAPALWRLEPGTDGSLESLTAAPGDAETLAPEPLGPGQVRIAIRATGLNFRDVLIALGMYPDPALMGTEGAGVVTATGPGVTHLAPGDRVMGLLSGAYAPVVVADARTVARMPEGWTFAQGASVPVVFLTAVYALRDLADVKPGERLLVHSAAGGVGMAAVQLARHWGVEVHGTASHGKWDALRALGLDDAHIASSRTLDFESAFRAASGGAGMDVVLNSLAREFVDASLRLLGPGGRFVEMGKTDVRDAERVAADHPGVGYRAFDLGEAGPERIGEMLAEVIALFEDGVLRHLPVTTWDVRRARDAFRHVSQARHTGKVVLTMPSGLDPEGTVLLTGGTGALGGIVARHVVGEWGVRRLLLVSRRGTDAPGAGELVHELEALGADVSVAACDVADREALTAVLDSIPAEHPLTAVVHTAGVLSDGTLPSMTAEDVEHVLRPKVDAAFLLDELTSTPGYDLAAFVMFSSAAAVFGGAGQGAYAAANATLDALAWRRRTAGLPALSLGWGLWAETSGMTGGLSDTDRSRLARSGATPMDSELTLSLLDAAMRRDDPALVPIALDVAALRAQQRDGMLAPLLSGLTRGSRVGGAPVNQRRAAAGGAGEADTDLGGRLAAMTPDDRVAHLRDLVRTHVATVLGHGTPSRVDLERAFRDTGFDSLTAVELRNRLNAATGLRLPATLVFDHPTPGELAGHLLDELATAAGGSWAEGTGSGDTASATDRQTTAALAELDRLEGVLASLAPAAGGRPELAARLRALAAALGDDGDDATDLDEASDDDLFSFIDKELGDSDFMANNEDKLRDYLKRVTAELQQNTRRLREIEGRTHEPVAIVGMACRLPGGVASPEDLWQLVAGDGDAISEFPQDRGWDVEGLYDPDPDASGRTYCRSGGFLHDAGEFDADFFGISPREALAMDPQQRLSLTTAWEAIESAGIDPTALKGSGLGVFVGGWHTGYTSGQTTAVQSPELEGHLVSGAALGFLSGRIAYVLGTDGPALTVDTACSSSLVALHLAVQALRKGECDMALAGGVTVMPNADLFVQFSRQRGLAADGRSKAFATSADGFGPAEGAGVLLVERLSDARRNGHRILAVVRGSAVNQDGASNGLTAPHGPSQQRVIRRALADARLAPGDVDVVEAHGTGTRLGDPIEAQALIATYGQEKSSEQPLRLGALKSNIGHTQAAAGVAGVIKMVQAMRHGLLPKTLHVDEPSDQIDWSAGTVELLTEAVDWPEKQDGGLRRAAVSSFGISGTNAHVVLEEAPAVEDSPAVEPPAGGGVVPWPVSAKTPAALDAQIGQLAAYADGRTDVDPAVAARALVDSRTAMEHRAVAVGDSREALRDALRMPEGLVRGTSSDVGRVAFVFPGQGTQWAGMGAELLDSSPEFAASMAECETALSRYVDWSLEAVVRQEPGAPTLDRVDVVQPVTFAVMVSLAKVWQHHGITPQAVVGHSQGEIAAAYVAGALTLDDAARVVTLRSKSIAAHLAGKGGMISLALDEAAVLKRLSDFDGLSVAAVNGPTATVVSGDPTQIEELARTCEADGVRARIIPVDYASHSRQVEIIEKELAEVLAGLAPQAPHVPFFSTLEGTWITEPVLDGTYWYRNLRHRVGFAPAVETLAVDGFTHFIEVSAHPVLTMTLPETVTGLGTLRREQGGQERLVTSLAEAWANGLTIDWAPILPTATGHHPELPTYAFQTERFWLQSSAPTSAADDWRYRVEWKPLTASGQADLSGRWIVAVGSEPEAELLGALKAAGAEVDVLEAGADDDREALAARLTALTTGDGFTGVVSLLDDLVPQVAWVQALGDAGIKAPLWSVTQGAVSVGRLDTPADPDRAMLWGLGRVVALEHPERWAGLVDLPAQPDAAALAHLVTALSGATGEDQIAIRTTGLHARRLARAPLHGRRPTRDWQPHGTVLITGGTGALGSHAARWMAHHGAEHLLLVSRSGEQAPGATQLTAELTASGARVTIAACDVADPHAMRTLLDAIPAETPLTAVVHTAGAPGGDPLDVTGPEDIARILGAKTSGAEVLDDLLRGTPLDAFVLYSSNAGVWGSGSQGVYAAANAHLDALAARRRARGETATSVAWGLWAGDGMGRGADDAYWQRRGIRPMSPDRALDELAKALSHDETFVAVADVDWERFAPAFTVSRPSLLLDGVPEARQALAAPVGAPAPGDAAVAPTGQSSALAAITALPEPERRPALLTLVRTHAAAVLGHSSPDRVAPGRAFTELGFDSLTAVQLRNQLSTVVGNRLPATTVFDHPTPAALAAHLHEAYLAPAEPAPTDWEGRVRRALAELPLDRLRDAGVLDTVLRLTGIEPEPGSGGSDGGAADPGAEPEASIDDLDAEALIRMALGPRNTMTSSNEQLVDALRASLKENEELRKESRRRADRRQEPMAIVGMSCRFAGGIRSPEDLWDAVAAGKDLVSEVPEERGWDIDSLYDPVPGRKGTTYVRNAAFLDDAAGFDAAFFGISPREALAMDPQQRQLLEASWEVFERAGIDPASVRGTDVGVYVGCGYQDYAPDIRVAPEGTGGYVVTGNSSAVASGRIAYSLGLEGPAVTVDTACSSSLVALHLALKGLRNGDCSTALVGGVAVLATPGAFIEFSSQQAMAADGRTKGFASAADGLAWGEGVAVLLLERLSDARRKGHRVLAVVRGSAINQDGASNGLTAPHGPSQQHLIRQALADARLTSSDVDVVEGHGTGTRLGDPIEAQALLATYGQGRAPGQPLRLGTLKSNIGHTQAASGVAGVIKMVQALRHGVLPKTLHVDEPTDQVDWSAGSVELLTEAVDWPERPGRLRRAGVSAFGVGGTNAHVVLEEAPAVEESPAVEPPAGGGVVPWPVSAKTSAALDAQIGQLAAYAEDRTDVDPAVAARALVDSRTAMEHRAVAVGDSREALRDALRMPEGLVRGTVTDPGRVAFVFPGQGTQWAGMGAELLDSSPEFAAAMAECETALSPYVDWSLEAVVRQAPSAPTLDRVDVVQPVTFAVMVSLAKVWQHHGITPEAVIGHSQGEIAAAYVAGALTLDDAARVVTLRSKSIAAHLAGKGGMISLALSEEATRQRIENLHGLSIAAVNGPTATVVSGDPTQIQELAQACEADGIRARIIPVDYASHSAHVETIENELADVLAGLSPQTPQVPFFSTLEGTWITEPALDGGYWYRNLRHRVGFAPAVETLATDEGFTHFIEVSAHPVLTMTLPDKVTGLATLRREDGGQHRLTTSLAEAWANGLALDWASLLPATGALSPAVPDLPTYAFQHRSYWISPAGPGEAPAHTASGREAVAETGLAWGPGAEDLDEEGRRSAVLAMVMRQAASVLRCDSPEEVPVDRPLREIGFDSLTAVDFRNRVNRLTGLQLPPTVVFQHPTPVALAERISDELAERNWAVAEPSDHEQAEEEKAAAPAGARSGADTGAGAGMFRALFRQAVEDDRYGEFLDVLAEASAFRPQFASPEACSERLDPVLLAGGPTDRAEGRAVLVGCTGTAANGGPHEFLRLSTSFQEERDFLAVPLPGYGTGTGTGTALLPADLDTALDAQARAILRAAGDAPVVLLGHSGGALLAHELAFRLERAHGAPPAGIVLVDPYPPGHQEPIEVWSRQLGEGLFAGELEPMSDARLLAMGRYARFLAGPRPGRSSAPVLLVRASEPLGDWQEERGDWRAHWDLPHTVADVPGDHFTMMRDHAPAVAEAVLSWLDAIEGIEGAGKMTDRPLNVDSGLWIRRFHPAPNSAVRLVCLPHAGGSASYFFRFSEELHPSVEALSVQYPGRQDRRAEPCLESVEELAEHVVAATEPWWQEGRLAFFGHSLGASVAFETARILEQRHGVRPEGLYVSGRRAPSLAPDRLVHQLDDRAFLAEIRRLSGTDERFLQDDELLRLVLPALRSDYKAAETYLHRPSAKLTCPVMALAGDRDPKAPLNEVAEWRRHTSGPFCLRAYSGGHFYLNDQWHEICNDISDHLLVTRGAPDARVVQPPTSLIEGAAKRWQNPR

SEQ ID NO: 7 - DNA, 1248 bp, Streptomyces venezuelae
gtgaaaagcgccttatccgacctcgcattcttcggcggccccgccgctttcgaccagccgctcctcgtggggcggcccaaccgcatcgaccgcgccaggctgtacgagcggctcgaccgggccctcgacagccagtggctgtccaacggcggcccgctcgtccgcgagttcgaggagcgcgtcgccgggctcgccggggtccggcatgccgtggccacctgcaacgccacggccgggctccagctcctcgcgcacgccgccggcctcaccggcgaagtgatcatgccgtcgatgacgttcgccgccaccccgcacgcactgcgctggatcggcctcaccccggtcttcgccgacatcgacccggacaccggcaacctcgacccggaccaggtggccgccgcggtcacaccccgcacctcggccgtcgtcggcgtccacctctggggccgcccctgcgccgccgaccagctgcggaaggtcgccgacgagcacggcctgcggctgtacttcgacgccgcgcacgccctcggctgcgcggtcgacggccggcccgccggcagcctcggcgacgccgaggtcttcagcttccacgccaccaaggccgtcaacgccttcgagggcggcgccgtcgtcaccgacgacgccgacctcgccgcccggatccgcgccctccacaacttcggcttcgacctgcccggcggcagccccgccggcgggaccaacgccaagatgagcgaggccgccgccgccatgggcctcacctccctcgacgcgtttcccgaggtcatcgaccggaaccggcgcaaccacgccgcctaccgcgagcacctcgcggacctccccggcgtcctcgtcgccgaccacgaccgccacggcctcaacaaccaccagtacgtgatcgtcgagatcgacgaggccaccaccggcatccaccgcgacctcgtcatggaggtcctgaaggccgaaggcgtgcacacccgcgcctacttctcgccgggctgccacgagctggagccgtaccgcgggcagccgcacgccccgctgccgcacaccgaacgcctcgccgcgcgcgtgctgtccctgccgaccggcaccgccatcggcgacgacgacatccgccgggtcgccgacctgctgcgtctctgcgcgacccgcggccgcgaactgaccgcgcgccaccgcgacacggcccccgccccgctcgcggccccccagacatccacgcccacgattggacgctcccgatga

SEQ ID NO: 8 - protein, 415 aa, Streptomyces venezuelae
MKSALSDLAFFGGPAAFDQPLLVGRPNRIDRARLYERLDRALDSQWLSNGGPLVREFEERVAGLAGVRHAVATCNATAGLQLLAHAAGLTGEVIMPSMTFAATPHALRWIGLTPVFADIDPDTGNLDPDQVAAAVTPRTSAVVGVHLWGRPCAADQLRKVADEHGLRLYFDAAHALGCAVDGRPAGSLGDAEVFSFHATKAVNAFEGGAVVTDDADLAARIRALHNFGFDLPGGSPAGGTNAKMSEAAAAMGLTSLDAFPEVIDRNRRNHAAYREHLADLPGVLVADHDRHGLNNHQYVIVEIDEATTGIHRDLVMEVLKAEGVHTRAYFSPGCHELEPYRGQPHAPLPHTERLAARVLSLPTGTAIGDDDIRRVADLLRLCATRGRELTARHRDTAPAPLAAPQTSTPTIGRSR

SEQ ID NO: 9 - DNA, 1458 bp, Streptomyces venezuelae
atgaccgcccccgccctttccgccaccgccccggccgaacgctgcgcgcaccccggagccgatctgggggcggcggtccacgccgtcggccagaccctcgccgccggcggcctcgtgccgcccgacgaggccggaacgaccgcccgccacctcgtccggctcgccgtgcgctacggcaacagccccttcaccccgctggaggaggcccgccacgacctgggcgtcgaccgggacgccttccggcgcctcctcgccctgttcgggcaggtcccggagctccgcaccgcggtcgagaccggccccgccggggcgtactggaagaacaccctgctcccgctcgaacagcgcggcgtcttcgacgcggcgctcgccaggaagcccgtcttcccgtacagcgtcggcctctaccccggcccgacctgcatgttccgctgccacttctgcgtccgtgtgaccggcgcccgctacgacccgtccgccctcgacgccggcaacgccatgttccggtcggtcatcgacgagatacccgcgggcaacccctcggcgatgtacttctccggcggcctggagccgctcaccaaccccggcctcgggagcctggccgcgcacgccaccgaccacggcctgcggcccaccgtctacacgaactccttcgcgctcaccgagcgcaccctggagcgccagcccggcctctggggcctgcacgccatccgcacctcgctctacggcctcaacgacgaggagtacgagcagaccaccggcaagaaggccgccttccgccgcgtccgcgagaacctgcgccgcttccagcagctgcgcgccgagcgcgagtcgccgatcaacctcggcttcgcctacatcgtgctcccgggccgtgcctcccgcctgctcgacctggtcgacttcatcgccgacctcaacgacgccgggcagggcaggacgatcgacttcgtcaacattcgcgaggactacagcggccgtgacgacggcaagctgccgcaggaggagcgggccgagctccaggaggccctcaacgccttcgaggagcgggtccgcgagcgcacccccggactccacatcgactacggctacgccctgaacagcctgcgcaccggggccgacgccgaactgctgcggatcaagcccgccaccatgcggcccaccgcgcacccgcaggtcgcggtgcaggtcgatctcctcggcgacgtgtacctgtaccgcgaggccggcttccccgacctggacggcgcgacccgctacatcgcgggccgcgtgacccccgacacctccctcaccgaggtcgtcagggacttcgtcgagcgcggcggcgaggtggcggccgtcgacggcgacgagtacttcatggacggcttcgatcaggtcgtcaccgcccgcctgaaccagctggagcgcgacgccgcggacggctgggaggaggcccgcggcttcctgcgctga

SEQ ID NO: 10 - protein, 485 aa, Streptomyces venezuelae
MTAPALSATAPAERCAHPGADLGAAVHAVGQTLAAGGLVPPDEAGTTARHLVRLAVRYGNSPFTPLEEARHDLGVDRDAFRRLLALFGQVPELRTAVETGPAGAYWKNTLLPLEQRGVFDAALARKPVFPYSVGLYPGPTCMFRCHFCVRVTGARYDPSALDAGNAMFRSVIDEIPAGNPSAMYFSGGLEPLTNPGLGSLAAHATDHGLRPTVYTNSFALTERTLERQPGLWGLHAIRTSLYGLNDEEYEQTTGKKAAFRRVRENLRRFQQLRAERESPINLGFAYIVLPGRASRLLDLVDFIADLNDAGQGRTIDFVNIREDYSGRDDGKLPQEERAELQEALNAFEERVRERTPGLHIDYGYALNSLRTGADAELLRIKPATMRPTAHPQVAVQVDLLGDVYLYREAGFPDLDGATRYIAGRVTPDTSLTEVVRDFVERGGEVAAVDGDEYFMDGFDQVVTARLNQLERDAADGWEEARGFLR

SEQ ID NO: 11 - DNA, 879 bp, Streptomyces venezuelae
atgaagggaatagtcctggccggcgggagcggaactcggctgcatccggcgacctcggtcatttcgaagcagattcttccggtctacaacaaaccgatgatctactatccgctgtcggttctcatgctcggcggtattcgcgagattcaaatcatctcgaccccccagcacatcgaactcttccagtcgcttctcggaaacggcaggcacctgggaatagaactcgactatgcggtccagaaagagcccgcaggaatcgcggacgcacttctcgtcggagccgagcacatcggcgacgacacctgcgccctgatcctgggcgacaacatcttccacgggcccggcctctacacgctcctgcgggacagcatcgcgcgcctcgacggctgcgtgctcttcggctacccggtcaaggaccccgagcggtacggcgtcgccgaggtggacgcgacgggccggctgaccgacctcgtcgagaagcccgtcaagccgcgctccaacctcgccgtcaccggcctctacctctacgacaacgacgtcgtcgacatcgccaagaacatccggccctcgccgcgcggcgagctggagatcaccgacgtcaaccgcgtctacctggagcggggccgggccgaactcgtcaacctgggccgcggcttcgcctggctggacaccggcacccacgactcgctcctgcgggccgcccagtacgtccaggtcctggaggagcggcagggcgtctggatcgcgggccttgaggagatcgccttccgcatgggcttcatcgacgccgaggcctgtcacggcctgggagaaggcctctcccgcaccgagtacggcagctatctgatggagatcgccggccgcgagggagccccgtga

SEQ ID NO: 12 - protein, 292 aa, Streptomyces venezuelae
MKGIVLAGGSGTRLHPATSVISKQILPVYNKPMIYYPLSVLMLGGIREIQIISTPQHIELFQSLLGNGRHLGIELDYAVQKEPAGIADALLVGAEHIGDDTCALILGDNIFHGPGLYTLLRDSIARLDGCVLFGYPVKDPERYGVAEVDATGRLTDLVEKPVKPRSNLAVTGLYLYDNDVVDIAKNIRPSPRGELEITDVNRVYLERGRAELVNLGRGFAWLDTGTHDSLLRAAQYVQVLEERQGVWIAGLEEIAFRMGFIDAEACHGLGEGLSRTEYGSYLMEIAGREGAP

SEQ ID NO: 13 - DNA, 1014 bp, Streptomyces venezuelae
gtgcggcttctggtgaccggaggtgcgggcttcatcggctcgcacttcgtgcggcagctcctcgccggggcgtaccccgacgtgcccgccgatgaggtgatcgtcctggacagcctcacctacgcgggcaaccgcgccaacctcgccccggtggacgcggacccgcgactgcgcttcgtccacggcgacatccgcgacgccggcctcctcgcccgggaactgcgcggcgtggacgccatcgtccacttcgcggccgagagccacgtggaccgctccatcgcgggcgcgtccgtgttcaccgagaccaacgtgcagggcacgcagacgctgctccagtgcgccgtcgacgccggcgtcggccgggtcgtgcacgtctccaccgacgaggtgtacgggtcgatcgactccggctcctggaccgagagcagcccgctggagcccaactcgccctacgcggcgtccaaggccggctccgacctcgttgcccgcgcctaccaccggacgtacggcctcgacgtacggatcacccgctgctgcaacaactacgggccgtaccagcaccccgagaagctcatccccctcttcgtgacgaacctcctcgacggcgggacgctcccgctgtacggcgacggcgcgaacgtccgcgagtgggtgcacaccgacgaccactgccggggcatcgcgctcgtcctcgcgggcggccgggccggcgagatctaccacatcggcggcggcctggagctgaccaaccgcgaactcaccggcatcctcctggactcgctcggcgccgactggtcctcggtccggaaggtcgccgaccgcaagggccacgacctgcgctactccctcgacggcggcgagatcgagcgcgagctcggctaccgcccgcaggtctccttcgcggacggcctcgcgcggaccgtccgctggtaccgggagaaccgcggctggtgggagccgctcaaggcgaccgccccgcagctgcccgccaccgccgtggaggtgtccgcgtga

SEQ ID NO: 14 - protein, 337 aa, Streptomyces venezuelae
MRLLVTGGAGFIGSHFVRQLLAGAYPDVPADEVIVLDSLTYAGNRANLAPVDADPRLRFVHGDIRDAGLLARELRGVDAIVHFAAESHVDRSIAGASVFTETNVQGTQTLLQCAVDAGVGRVVHVSTDEVYGSIDSGSWTESSPLEPNSPYAASKAGSDLVARAYHRTYGLDVRITRCCNNYGPYQHPEKLIPLFVTNLLDGGTLPLYGDGANVREWVHTDDHCRGIALVLAGGRAGEIYHIGGGLELTNRELTGILLDSLGADWSSVRKVADRKGHDLRYSLDGGEIERELGYRPQVSFADGLARTVRWYRENRGWWEPLKATAPQLPATAVEVSA

SEQ ID NO: 15 - DNA, 1140 bp, Streptomyces venezuelae
gtgagcagccgcgccgagaccccccgcgtccccttcctcgacctcaaggccgcctacgaggagctccgcgcggagaccgacgccgcgatcgcccgcgtcctcgactcggggcgctacctcctcggacccgaactcgaaggattcgaggcggagttcgccgcgtactgcgagacggaccacgccgtcggcgtgaacagcgggatggacgccctccagctcgccctccgcggcctcggcatcggacccggggacgaggtgatcgtcccctcgcacacgtacatcgccagctggctcgcggtgtccgccaccggcgcgacccccgtgcccgtcgagccgcacgaggaccaccccaccctggacccgctgctcgtcgagaaggcgatcaccccccgcacccgggcgctcctccccgtccacctctacgggcaccccgccgacatggacgccctccgcgagctcgcggaccggcacggcctgcacatcgtcgaggacgccgcgcaggcccacggcgcccgctaccggggccggcggatcggcgccgggtcgtcggtggccgcgttcagcttctacccgggcaagaacctcggctgcttcggcgacggcggcgccgtcgtcaccggcgaccccgagctcgccgaacggctccggatgctccgcaactacggctcgcggcagaagtacagccacgagacgaagggcaccaactcccgcctggacgagatgcaggccgccgtgctgcggatccggctcgcccacctggacagctggaacggccgcaggtcggcgctggccgcggagtacctctccgggctcgccggactgcccggcatcggcctgccggtgaccgcgcccgacaccgacccggtctggcacctcttcaccgtgcgcaccgagcgccgcgacgagctgcgcagccacctcgacgcccgcggcatcgacaccctcacgcactacccggtacccgtgcacctctcgcccgcctacgcgggcgaggcaccgccggaaggctcgctcccgcgggccgagagcttcgcgcggcaggtcctcagcctgccgatcggcccgcacctggagcgcccgcaggcgctgcgggtgatcgacgccgtgcgcgaatgggccgagcgggtcgaccaggcctag

SEQ ID NO: 16 - protein, 379 aa, Streptomyces venezuelae
MSSRAETPRVPFLDLKAAYEELRAETDAAIARVLDSGRYLLGPELEGFEAEFAAYCETDHAVGVNSGMDALQLALRGLGIGPGDEVIVPSHTYIASWLAVSATGATPVPVEPHEDHPTLDPLLVEKAITPRTRALLPVHLYGHPADMDALRELADRHGLHIVEDAAQAHGARYRGRRIGAGSSVAAFSFYPGKNLGCFGDGGAVVTGDPELAERLRMLRNYGSRQKYSHETKGTNSRLDEMQAAVLRIRLAHLDSWNGRRSALAAEYLSGLAGLPGIGLPVTAPDTDPVWHLFTVRTERRDELRSHLDARGIDTLTHYPVPVHLSPAYAGEAPPEGSLPRAESFARQVLSLPIGPHLERPQALRVIDAVREWAERVDQA

SEQ ID NO: 17 - DNA, 714 bp, Streptomyces venezuelae
gtgtacgaagtcgaccacgccgacgtctacgacctcttctacctgggtcgcggcaaggactacgccgccgaggcctccgacatcgccgacctggtgcgctcccgtacccccgaggcctcctcgctcctggacgtggcctgcggtacgggcacgcatctggagcacttcaccaaggagttcggcgacaccgccggcctggagctgtccgaggacatgctcacccacgcccgcaagcggctgcccgacgccacgctccaccagggcgacatgcgggacttccggctcggccggaagttctccgccgtggtcagcatgttcagctccgtcggctacctgaagacgaccgaggaactcggcgcggccgtcgcctcgttcgcggagcacctggagcccggtggcgtcgtcgtcgtcgagccgtggtggttcccggagaccttcgccgacggctgggtcagcgccgacgtcgtccgccgtgacgggcgcaccgtggcccgtgtctcgcactcggtgcgggaggggaacgcgacgcgcatggaggtccacttcaccgtggccgacccgggcaagggcgtgcggcacttctccgacgtccatctcatcaccctgttccaccaggccgagtacgaggccgcgttcacggccgccgggctgcgcgtcgagtacctggagggcggcccgtcgggccgtggcctcttcgtcggcgtccccgcctga

SEQ ID NO: 18 - protein, 237 aa, Streptomyces venezuelae
MYEVDHADVYDLFYLGRGKDYAAEASDIADLVRSRTPEASSLLDVACGTGTHLEHFTKEFGDTAGLELSEDMLTHARKRLPDATLHQGDMRDFRLGRKFSAVVSMFSSVGYLKTTEELGAAVASFAEHLEPGGVVVVEPWWFPETFADGWVSADVVRRDGRTVARVSHSVREGNATRMEVHFTVADPGKGVRHFSDVHLITLFHQAEYEAAFTAAGLRVEYLEGGPSGRGLFVGVPA

SEQ ID NO: 19 - DNA, 1281 bp, Streptomyces venezuelae
atgcgcgtcctgctgacctcgttcgcacatcacacgcactactacggcctggtgcccctggcctgggcgctgctcgccgccgggcacgaggtgcgggtcgccagccagcccgcgctcacggacaccatcaccgggtccgggctcgccgcggtgccggtcggcaccgaccacctcatccacgagtaccgggtgcggatggcgggcgagccgcgcccgaaccatccggcgatcgccttcgacgaggcccgtcccgagccgctggactgggaccacgccctcggcatcgaggcgatcctcgccccgtacttccatctgctcgccaacaacgactcgatggtcgacgacctcgtcgacttcgcccggtcctggcagccggacctggtgctgtgggagccgacgacctacgcgggcgccgtcgccgcccaggtcaccggtgccgcgcacgcccgggtcctgtgggggcccgacgtgatgggcagcgcccgccgcaagttcgtcgcgctgcgggaccggcagccgcccgagcaccgcgaggaccccaccgcggagtggctgacgtggacgctcgaccggtacggcgcctccttcgaagaggagctgctcaccggccagttcacgatcgacccgaccccgccgagcctgcgcctcgacacgggcctgccgaccgtcgggatgcgttatgttccgtacaacggcacgtcggtcgtgccggactggctgagtgagccgcccgcgcggccccgggtctgcctgaccctcggcgtctccgcgcgtgaggtcctcggcggcgacggcgtctcgcagggcgacatcctggaggcgctcgccgacctcgacatcgagctcgtcgccacgctcgacgcgagtcagcgcgccgagatccgcaactacccgaagcacacccggttcacggacttcgtgccgatgcacgcgctcctgccgagctgctcggcgatcatccaccacggcggggcgggcacctacgcgaccgccgtgatcaacgcggtgccgcaggtcatgctcgccgagctgtgggacgcgccggtcaaggcgcgggccgtcgccgagcagggggcggggttcttcctgccgccggccgagctcacgccgcaggccgtgcgggacgccgtcgtccgcatcctcgacgacccctcggtcgccaccgccgcgcaccggctgcgcgaggagaccttcggcgaccccaccccggccgggatcgtccccgagctggagcggctcgccgcgcagcaccgccgcccgccggccgacgcccggcactga

SEQ ID NO: 20 - protein, 426 aa, Streptomyces venezuelae
MRVLLTSFAHHTHYYGLVPLAWALLAAGHEVRVASQPALTDTITGSGLAAVPVGTDHLIHEYRVRMAGEPRPNHPAIAFDEARPEPLDWDHALGIEAILAPYFHLLANNDSMVDDLVDFARSWQPDLVLWEPTTYAGAVAAQVTGAAHARVLWGPDVMGSARRKFVALRDRQPPEHREDPTAEWLTWTLDRYGASFEEELLTGQFTIDPTPPSLRLDTGLPTVGMRYVPYNGTSVVPDWLSEPPARPRVCLTLGVSAREVLGGDGVSQGDILEALADLDIELVATLDASQRAEIRNYPKHTRFTDFVPMHALLPSCSAIIHHGGAGTYATAVINAVPQVMLAELWDAPVKARAVAEQGAGFFLPPAELTPQAVRDAVVRILDDPSVATAAHRLREETFGDPTPAGIVPELERLAAQHRRPPADARH

SEQ ID NO: 21 - DNA, 1209 bp, Streptomyces venezuelae
gtgaccgacgacctgacgggggccctcacgcagcccccgctgggccgcaccgtccgcgcggtggccgaccgtgaactcggcacccacctcctggagacccgcggcatccactggatccacgccgcgaacggcgacccgtacgccaccgtgctgcgcggccaggcggacgacccgtatcccgcgtacgagcgggtgcgtgcccgcggcgcgctctccttcagcccgacgggcagctgggtcaccgccgatcacgccctggcggcgagcatcctctgctcgacggacttcggggtctccggcgccgacggcgtcccggtgccgcagcaggtcctctcgtacggggagggctgtccgctggagcgcgagcaggtgctgccggcggccggtgacgtgccggagggcgggcagcgtgccgtggtcgaggggatccaccgggagacgctggagggtctcgcgccggacccgtcggcgtcgtacgccttcgagctgctgggcggtttcgtccgcccggcggtgacggccgctgccgccgccgtgctgggtgttcccgcggaccggcgcgcggacttcgcggatctgctggagcggctccggccgctgtccgacagcctgctggccccgcagtccctgcggacggtacgggcggcggacggcgcgctggccgagctcacggcgctgctcgccgattcggacgactcccccggggccctgctgtcggcgctcggggtcaccgcagccgtccagctcaccgggaacgcggtgctcgcgctcctcgcgcatcccgagcagtggcgggagctgtgcgaccggcccgggctcgcggcggccgcggtggaggagaccctccgctacgacccgccggtgcagctcgacgcccgggtggtccgcggggagacggagctggcgggccggcggctgccggccggggcgcatgtcgtcgtcctgaccgccgcgaccggccgggacccggaggtcttcacggacccggagcgcttcgacctcgcgcgccccgacgccgccgcgcacctcgcgctgcaccccgccggtccgtacggcccggtggcgtccctggtccggcttcaggcggaggtcgcgctgcggaccctggccgggcgtttccccgggctgcggcaggcgggggacgtgctccgcccccgccgcgcgcctgtcggccgcgggccgctgagcgtcccggtcagcagctcctga

SEQ ID NO: 22 - protein, 402 aa, Streptomyces venezuelae
MTDDLTGALTQPPLGRTVRAVADRELGTHLLETRGIHWIHAANGDPYATVLRGQADDPYPAYERVRARGALSFSPTGSWVTADHALAASILCSTDFGVSGADGVPVPQQVLSYGEGCPLEREQVLPAAGDVPEGGQRAVVEGIHRETLEGLAPDPSASYAFELLGGFVRPAVTAAAAAVLGVPADRRADFADLLERLRPLSDSLLAPQSLRTVRAADGALAELTALLADSDDSPGALLSALGVTAAVQLTGNAVLALLAHPEQWRELCDRPGLAAAAVEETLRYDPPVQLDARVVRGETELAGRRLPAGAHVVVLTAATGRDPEVFTDPERFDLARPDAAAHLALHPAGPYGPVASLVRLQAEVALRTLAGRFPGLRQAGDVLRPRRAPVGRGPLSVPVSSS

SEQ ID NO: 23 - DNA, 2430 bp, Streptomyces venezuelae
gtgacaggtaagacccgaataccgcgtgtccgccgcggccgcaccacgcccagggccttcaccctggccgtcgtcggcaccctgctggcgggcaccaccgtggcggccgccgctcccggcgccgccgacacggccaatgttcagtacacgagccgggcggcggagctcgtcgcccagatgacgctcgacgagaagatcagcttcgtccactgggcgctggaccccgaccggcagaacgtcggctaccttcccggcgtgccgcgtctgggcatcccggagctgcgtgccgccgacggcccgaacggcatccgcctggtggggcagaccgccaccgcgctgcccgcgccggtcgccctggccagcaccttcgacgacaccatggccgacagctacggcaaggtcatgggccgcgacggtcgcgcgctcaaccaggacatggtcctgggcccgatgatgaacaacatccgggtgccgcacggcggccggaactacgagaccttcagcgaggaccccctggtctcctcgcgcaccgcggtcgcccagatcaagggcatccagggtgcgggtctgatgaccacggccaagcacttcgcggccaacaaccaggagaacaaccgcttctccgtgaacgccaatgtcgacgagcagacgctccgcgagatcgagttcccggcgttcgaggcgtcctccaaggccggcgcggcctccttcatgtgtgcctacaacggcctcaacgggaagccgtcctgcggcaacgacgagctcctcaacaacgtgctgcgcacgcagtggggcttccagggctgggtgatgtccgactggctcgccaccccgggcaccgacgccatcaccaagggcctcgaccaggagatgggcgtcgagctccccggcgacgtcccgaagggcgagccctcgccgccggccaagttcttcggcgaggcgctgaagacggccgtcctgaacggcacggtccccgaggcggccgtgacgcggtcggcggagcggatcgtcggccagatggagaagttcggtctgctcctcgccactccggcgccgcggcccgagcgcgacaaggcgggtgcccaggcggtgtcccgcaaggtcgccgagaacggcgcggtgctcctgcgcaacgagggccaggccctgccgctcgccggtgacgccggcaagagcatcgcggtcatcggcccgacggccgtcgaccccaaggtcaccggcctgggcagcgcccacgtcgtcccggactcggcggcggcgccactcgacaccatcaaggcccgcgcgggtgcgggtgcgacggtgacgtacgagacgggtgaggagaccttcgggacgcagatcccggcggggaacctcagcccggcgttcaaccagggccaccagctcgagccgggcaaggcgggggcgctgtacgacggcacgctgaccgtgcccgccgacggcgagtaccgcatcgcggtccgtgccaccggtggttacgccacggtgcagctcggcagccacaccatcgaggccggtcaggtctacggcaaggtgagcagcccgctcctcaagctgaccaagggcacgcacaagctcacgatctcgggcttcgcgatgagtgccaccccgctctccctggagctgggctgggtgacgccggcggcggccgacgcgacgatcgcgaaggccgtggagtcggcgcggaaggcccgtacggcggtcgtcttcgcctacgacgacggcaccgagggcgtcgaccgtccgaacctgtcgctgccgggtacgcaggacaagctgatctcggctgtcgcggacgccaacccgaacacgatcgtggtcctcaacaccggttcgtcggtgctgatgccgtggctgtccaagacccgcgcggtcctggacatgtggtacccgggccaggcgggcgccgaggccaccgccgcgctgctctacggtgacgtcaacccgagcggcaagctcacgcagagcttcccggccgccgagaaccagcacgcggtcgccggcgacccgacaagctacccgggcgtcgacaaccagcagacgtaccgcgagggcatccacgtcgggtaccgctggttcgacaaggagaacgtcaagccgctgttcccgttcgggcacggcctgtcgtacacctcgttcacgcagagcgccccgaccgtcgtgcgtacgtccacgggtggtctgaaggtcacggtcacggtccgcaacagcgggaagcgcgccggccaggaggtcgtccaggcgtacctcggtgccagcccgaacgtgacggctccgcaggcgaagaagaagctcgtgggctacacgaaggtctcgctcgccgcgggcgaggcgaagacggtgacggtgaacgtcgaccgccgtcagctgcagaccggttcgtcctccgccgacctgcggggcagcgccacggtcaacgtctggtga

SEQ ID NO: 24 - protein, 809 aa, Streptomyces venezuelae
MTGKTRIPRVRRGRTTPRAFTLAVVGTLLAGTTVAAAAPGAADTANVQYTSRAAELVAQMTLDEKISFVHWALDPDRQNVGYLPGVPRLGIPELRAADGPNGIRLVGQTATALPAPVALASTFDDTMADSYGKVMGRDGRALNQDMVLGPMMNNIRVPHGGRNYETFSEDPLVSSRTAVAQIKGIQGAGLMTTAKHFAANNQENNRFSVNANVDEQTLREIEFPAFEASSKAGAASFMCAYNGLNGKPSCGNDELLNNVLRTQWGFQGWVMSDWLATPGTDAITKGLDQEMGVELPGDVPKGEPSPPAKFFGEALKTAVLNGTVPEAAVTRSAERIVGQMEKFGLLLATPAPRPERDKAGAQAVSRKVAENGAVLLRNEGQALPLAGDAGKSIAVIGPTAVDPKVTGLGSAHVVPDSAAAPLDTIKARAGAGATVTYETGEETFGTQIPAGNLSPAFNQGHQLEPGKAGALYDGTLTVPADGEYRIAVRATGGYATVQLGSHTIEAGQVYGKVSSPLLKLTKGTHKLTISGFAMSATPLSLELGWVTPAAADATIAKAVESARKARTAVVFAYDDGTEGVDRPNLSLPGTQDKLISAVADANPNTIVVLNTGSSVLMPWLSKTRAVLDMWYPGQAGAEATAALLYGDVNPSGKLTQSFPAAENQHAVAGDPTSYPGVDNQQTYREGIHVGYRWFDKENVKPLFPFGHGLSYTSFTQSAPTVVRTSTGGLKVTVTVRNSGKRAGQEVVQAYLGASPNVTAPQAKKKLVGYTKVSLAAGEAKTVTVNVDRRQLQTGSSSADLRGSATVNVW

SEQ ID NO: 25 - protein, 9 aa, Artificial Sequence
  A consensus sequence.
LLDVACGTG

SEQ ID NO: 26 - DNA, 1011 bp, Streptomyces venezuelae
atggcaatgcgcgactccataccgaggcgagcggaccgcgacacccttcgccgcgaattaggccagaacttccttcaggacgacagagccgtgcgcaatctcgtcacgcatgtcgagggggacggtaggaacgttctcgaaatcggccccggaaagggcgcgataaccgaggagttggtgcgctccttcgacaccgtgacggtcgtggagatggacccgcactgggccgcgcatgtgcggcggaaattcgaaggggagagggtcaccgtattccagggtgatttcctcgacttccgcattccgcgcgatatcgacaccgtcgtcggaaacgttcccttcggcatcacgacccagattctccggagtctcctggaatcgacgaactggcagtcggcggccctgatagtgcagtgggaggtcgcccgcaaacgcgccggtcgcagcggcggatcgctcctcacgacctcctgggccccctggtacgagttcgcggtccacgaccgcgtccgcgcctcgtcgttccgtccgatgccccgcgtcgacggcggcgtcctgacgatcaggcgacgcccccagcccctgctgcccgagagcgcgagccgcgccttccagaacttcgccgaagccgtcttcaccggccccggacggggcctcgcggagatcctccggcgccacatccccaagcggacctaccgttccctcgccgaccgccacggaattccggacggcggactgccgaaggacctcacgctcacccaatggatcgcccttttccaggcctcccagccgagttacgcgccgggggcgcccggcacgcgcatgccgggccagggcggtggcgccggcggcagggactatgactcggagacgagcagggccgccgtgcccgggagccgcagatacggccccacgcgcggcggcgaaccctgcgcaccccgcgcacaggtccggcagaccaagggccgccagggcgcgcgaggctcgtcgtacggacgccgcacgggccgttag

SEQ ID NO: 27 - protein, 336 aa, Streptomyces venezuelae
MAMRDSIPRRADRDTLRRELGQNFLQDDRAVRNLVTHVEGDGRNVLEIGPGKGAITEELVRSFDTVTVVEMDPHWAAHVRRKFEGERVTVFQGDFLDFRIPRDIDTVVGNVPFGITTQILRSLLESTNWQSAALIVQWEVARKRAGRSGGSLLTTSWAPWYEFAVHDRVRASSFRPMPRVDGGVLTIRRRPQPLLPESASRAFQNFAEAVFTGPGRGLAEILRRHIPKRTYRSLADRHGIPDGGLPKDLTLTQWIALFQASQPSYAPGAPGTRMPGQGGGAGGRDYDSETSRAAVPGSRRYGPTRGGEPCAPRAQVRQTKGRQGARGSSYGRRTGR

SEQ ID NO: 28 - DNA, 969 bp, Streptomyces venezuelae
atggcattttccccgcagggcggccgacacgagctcggtcagaacttcctcgtcgaccggtcagtgatcgacgagatcgacggcctggtggccaggaccaagggtccgatactggagatcggtccgggtgacggcgccctgaccctgccgctgagcaggcacggcaggccgatcaccgccgtcgagctcgacggccggcgcgcgcagcgcctcggtgcccgcacccccggtcatgtgaccgtggtgcaccacgacttcctgcagtacccgctgccgcgcaacccgcatgtggtcgtcggcaacgtccccttccatctgacgacggcgatcatgcggcggctgctcgacgcccagcactggcacaccgccgtcctcctcgtccagtgggaggtcgcccggcgccgggccggcgtcggcgggtcgacgctgctgacggccggctgggcgccctggtacgagttcgacctgcactcccgggtccccgcgcgggccttccgtccgatgccgggcgtggacggaggagtactggccatccggcggcggtccgcgccgctcgtgggccaggtgaagacgtaccaggacttcgtacgccaggtgttcaccggcaaggggaacgggctgaaggagatcctgcggcggaccgggcggatctcgcagcgggacctggcgacctggctgcggaggaacgagatctcgccgcacgcgctgcccaaggacctgaagcccgggcagtgggcgtcgctgtgggagctgaccggcggcacggccgacggatccttcgacggtacggcgggcggtggcgcggccggatcgcacggggcggctcgggtcggggccggtcacccgggcggccgggtgtccgcgagccggcggggcgtgccgcaggcgcggcgcggccgggggcatgcggtacggagctccacggggaccgagccgaggtggggcagggggcgggcggagagcgcgtga

SEQ ID NO: 29 - protein, 322 aa, Streptomyces venezuelae
MAFSPQGGRHELGQNFLVDRSVIDEIDGLVARTKGPILEIGPGDGALTLPLSRHGRPITAVELDGRRAQRLGARTPGHVTVVHHDFLQYPLPRNPHVVVGNVPFHLTTAIMRRLLDAQHWHTAVLLVQWEVARRRAGVGGSTLLTAGWAPWYEFDLHSRVPARAFRPMPGVDGGVLAIRRRSAPLVGQVKTYQDFVRQVFTGKGNGLKEILRRTGRISQRDLATWLRRNEISPHALPKDLKPGQWASLWELTGGTADGSFDGTAGGGAAGSHGAARVGAGHPGGRVSASRRGVPQARRGRGHAVRSSTGTEPRWGRGRAESA

SEQ ID NO: 30 - DNA, 13842 bp, Streptomyces venezuelae
atgtcttcagccggaattaccaggaccggtgcgagaacaccggtgacagggcgtggggcggcagcgtgggacacgggggaagtgcgggtccgacgggggttgccccctgccggccccgatcatgcggagcactccttctctcgtgctcctaccggtgatgtgcgcgccgaattgattcgtggagagatgtcgacagtgtccaagagtgagtccgaggaattcgtgtccgtgtcgaacgacgccggttccgcgcacggcacagcggaacccgtcgccgtcgtcggcatctcctgccgggtgcccggcgcccgggacccgagagagttctgggaactcctggcggcaggcggccaggccgtcaccgacgtccccgcggaccgctggaacgccggcgacttctacgacccggaccgctccgcccccggccgctcgaacagccggtggggcgggttcatcgaggacgtcgaccggttcgacgccgccttcttcggcatctcgccccgcgaggccgcggagatggacccgcagcagcggctcgccctggagctgggctgggaggccctggagcgcgccgggatcgacccgtcctcgctcaccggcacccgcaccggcgtcttcgccggcgccatctgggacgactacgccaccctgaagcaccgccagggcggcgccgcgatcaccccgcacaccgtcaccggcctccaccgcggcatcatcgcgaaccgactctcgtacacgctcgggctccgcggccccagcatggtcgtcgactccggccagtcctcgtcgctcgtcgccgtccacctcgcgtgcgagagcctgcggcgcggcgagtccgagctcgccctcgccggcggcgtctcgctcaacctggtgccggacagcatcatcggggcgagcaagttcggcggcctctcccccgacggccgcgcctacaccttcgacgcgcgcgccaacggctacgtacgcggcgagggcggcggtttcgtcgtcctgaagcgcctctcccgggccgtcgccgacggcgacccggtgctcgccgtgatccggggcagcgccgtcaacaacggcggcgccgcccagggcatgacgacccccgacgcgcaggcgcaggaggccgtgctccgcgaggcccacgagcgggccgggaccgcgccggccgacgtgcggtacgtcgagctgcacggcaccggcacccccgtgggcgacccgatcgaggccgctgcgctcggcgccgccctcggcaccggccgcccggccggacagccgctcctggtcggctcggtcaagacgaacatcggccacctggagggcgcggccggcatcgccggcctcatcaaggccgtcctggcggtccgcggtcgcgcgctgcccgccagcctgaactacgagaccccgaacccggcgatcccgttcgaggaactgaacctccgggtgaacacggagtacctgccgtgggagccggagcacgacgggcagcggatggtcgtcggcgtgtcctcgttcggcatgggcggcacgaacgcgcatgtcgtgctcgaagaggcccccgggggttgtcgaggtgcttcggtcgtggagtcgacggtcggcgggtcggcggtcggcggcggtgtggtgccgtgggtggtgtcggcgaagtccgctgccgcgctggacgcgcagatcgagcggcttgccgcgttcgcctcgcgggatcgtacggatggtgtcgacgcgggcgctgtcgatgcgggtgctgtcgatgcgggtgctgtcgctcgcgtactggccggcgggcgtgctcagttcgagcaccgggccgtcgtcgtcggcagcgggccggacgatctggcggcagcgctggccgcgcctgagggtctggtccggggcgtggcttccggtgtcgggcgagtggcgttcgtgttccccgggcagggcacgcagtgggccggcatgggtgccgaactgctggactcttccgcggtgttcgcggcggccatggccgaatgcgaggccgcactctccccgtacgtcgactggtcgctggaggccgtcgtacggcaggcccccggtgcgcccacgctggagcgggtcgatgtcgtgcagcctgtgacgttcgccgtcatggtctcgctggctcgcgtgtggcagcaccacggggtgacgccccaggcggtcgtcggccactcgcagggcgagatcgccgccgcgtacgtcgccggtgccctgagcctggacgacgccgctcgtgtcgtgaccctgcgcagcaagtccatcgccgcccacctcgccggcaagggcggcatgctgtccctcgcgctgagcgaggacgccgtcctggagcgactggccgggttcgacgggctgtccgtcgccgctgtgaacgggcccaccgccaccgtggtctccggtgaccccgtacagatcgaagagcttgctcgggcgtgtgaggccgatggggtccgtgcgcgggtcattcccgtcgactacgcgtcccacagccggcaggtcgagatcatcgagagcgagctcgccgaggtcctcgccgggctcagcccgcaggctccgcgcgtgccgttcttctcgacactcgaaggcgcctggatcaccgagcccgtgctcgacggcggctactggtaccgcaacctgcgccatcgtgtgggcttcgccccggccgtcgagaccctggccaccgacgagggcttcacccacttcgtcgaggtcagcgcccaccccgtcctcaccatggccctccccgggaccgtcaccggtctggcgaccctgcgtcgcgacaacggcggtcaggaccgcctagtcgcctccctcgccgaagcatgggccaacggactcgcggtcgactggagcccgctcctcccctccgcgaccggccaccactccgacctccccacctacgcgttccagaccgagcgccactggctgggcgagatcgaggcgctcgccccggcgggcgagccggcggtgcagcccgccgtcctccgcacggaggcggccgagccggcggagctcgaccgggacgagcagctgcgcgtgatcctggacaaggtccgggcgcagacggcccaggtgctggggtacgcgacaggcgggcagatcgaggtcgaccggaccttccgtgaggccggttgcacctccctgaccggcgtggacctgcgcaaccggatcaacgccgccttcggcgtacggatggcgccgtccatgatcttcgacttccccacccccgaggctctcgcggagcagctgctcctcgtcgtgcacggggaggcggcggcgaacccggccggtgcggagccggctccggtggcggcggccggtgccgtcgacgagccggtggcgatcgtcggcatggcctgccgcctgcccggtggggtcgcctcgccggaggacctgtggcggctggtggccggcggcggggacgcgatctcggagttcccgcaggaccgcggctgggacgtggaggggctgtaccacccggatccggagcaccccggcacgtcgtacgtccgccagggcggtttcatcgagaacgtcgccggcttcgacgcggccttcttcgggatctcgccgcgcgaggccctcgccatggacccgcagcagcggctcctcctcgaaacctcctgggaggccgtcgaggacgccgggatcgacccgacctccctgcggggacggcaggtcggcgtcttcactggggcgatgacccacgagtacgggccgagcctgcgggacggcggggaaggcctcgacggctacctgctgaccggcaacacggccagcgtgatgtcgggccgcgtctcgtacacactcggccttgagggccccgccctgacggtggacacggcctgctcgtcgtcgctggtcgccctgcacctcgccgtgcaggccctgcgcaagggcgaggtcgacatggcgctcgccggcggcgtggccgtgatgcccacgcccgggatgttcgtcgagttcagccggcagcgcgggctggccggggacggccggtcgaaggcgttcgccgcgtcggcggacggcaccagctggtccgagggcgtcggcgtcctcctcgtcgagcgcctgtcggacgcccgccgcaacggacaccaggtcctcgcggtcgtccgcggcagcgccttgaaccaggacggcgcgagcaacggcctcacggctccgaacgggccctcgcagcagcgcgtcatccggcgcgcgctggcggacgcccggctgacgacctccgacgtggacgtcgtcgaggcacacggcacgggcacgcgactcggcgacccgatcgaggcgcaggccctgatcgccacctacggccagggccgtgacgacgaacagccgctgcgcctcgggtcgttgaagtccaacatcgggcacacccaggccgcggccggcgtctccggtgtcatcaagatggtccaggcgatgcgccacggactgctgccgaagacgctgcacgtcgacgagccctcggaccagatcgactggtcggctggcgccgtggaactcctcaccgaggccgtcgactggccggagaagcaggacggcgggctgcgccgggccgccgtctcctccttcgggatcagcggcaccaatgcgcatgtggtgctcgaagaggccccggtggttgtcgagggtgcttcggtcgtcgagccgtcggttggcgggtcggcggtcggcggcggtgtgacgccttgggtggtgtcggcgaagtccgctgccgcgctcgacgcgcagatcgagcggcttgccgcattcgcctcgcgggatcgtacggatgacgccgacgccggtgctgtcgacgcgggcgctgtcgctcacgtactggctgacgggcgtgctcagttcgagcaccgggccgtcgcgctcggcgccggggcggacgacctcgtacaggcgctggccgatccggacgggctgatacgcggaacggcttccggtgtcgggcgagtggcgttcgtgttccccggtcagggcacgcagtgggctggcatgggtgccgaactgctggactcttccgcggtgttcgcggcggccatggccgagtgtgaggccgcgctgtccccgtacgtcgactggtcgctggaggccgtcgtacggcaggcccccggtgcgcccacgctggagcgggtcgatgtcgtgcagcctgtgacgttcgccgtcatggtctcgctggctcgcgtgtggcagcaccacggtgtgacgccccaggcggtcgtcggccactcgcagggcgagatcgccgccgcgtacgtcgccggagccctgcccctggacgacgccgcccgcgtcgtcaccctgcgcagcaagtccatcgccgcccacctcgccggcaagggcggcatgctgtccctcgcgctgaacgaggacgccgtcctggagcgactgagtgacttcgacgggctgtccgtcgccgccgtcaacgggcccaccgccactgtcgtgtcgggtgaccccgtacagatcgaagagcttgctcaggcgtgcaaggcggacggattccgcgcgcggatcattcccgtcgactacgcgtcccacagccggcaggtcgagatcatcgagagcgagctcgcccaggtcctcgccggtctcagcccgcaggccccgcgcgtgccgttcttctcgacgctcgaaggcacctggatcaccgagcccgtcctcgacggcacctactggtaccgcaacctccgtcaccgcgtcggcttcgcccccgccatcgagaccctggccgtcgacgagggcttcacgcacttcgtcgaggtcagcgcccaccccgtcctcaccatgaccctccccgagaccgtcaccggcctcggcaccctccgtcgcgaacagggaggccaagagcgtctggtcacctcgctcgccgaggcgtgggtcaacgggcttcccgtggcatggacttcgctcctgcccgccacggcctcccgccccggtctgcccacctacgccttccaggccgagcgctactggctcgagaacactcccgccgccctggccaccggcgacgactggcgctaccgcatcgactggaagcgcctcccggccgccgaggggtccgagcgcaccggcctgtccggccgctggctcgccgtcacgccggaggaccactccgcgcaggccgccgccgtgctcaccgcgctggtcgacgccggggcgaaggtcgaggtgctgacggccggggcggacgacgaccgtgaggccctcgccgcccggctcaccgcactgacgaccggtgacggcttcaccggcgtggtctcgctcctcgacggactcgtaccgcaggtcgcctgggtccaggcgctcggcgacgccggaatcaaggcgcccctgtggtccgtcacccagggcgcggtctccgtcggacgtctcgacacccccgccgaccccgaccgggccatgctctggggcctcggccgcgtcgtcgcccttgagcaccccgaacgctgggccggcctcgtcgacctccccgcccagcccgatgccgccgccctcgcccacctcgtcaccgcactctccggcgccaccggcgaggaccagatcgccatccgcaccaccggactccacgcccgccgcctcgcccgcgcacccctccacggacgtcggcccacccgcgactggcagccccacggcaccgtcctcatcaccggcggcaccggagccctcggcagccacgccgcacgctggatggcccaccacggagccgaacacctcctcctcgtcagccgcagcggcgaacaagcccccggagccacccaactcaccgccgaactcaccgcatcgggcgcccgcgtcaccatcgccgcctgcgacgtcgccgacccccacgccatgcgcaccctcctcgacgccatccccgccgagacgcccctcaccgccgtcgtccacaccgccggcgcgctcgacgacggcatcgtggacacgctgaccgccgagcaggtccggcgggcccaccgtgcgaaggccgtcggcgcctcggtgctcgacgagctgacccgggacctcgacctcgacgcgttcgtgctcttctcgtccgtgtcgagcactctgggcatccccggtcagggcaactacgccccgcacaacgcctacctcgacgccctcgcggctcgccgccgggccaccggccggtccgccgtctcggtggcctggggaccgtgggacggtggcggcatggccgccggtgacggcgtggccgagcggctgcgcaaccacggcgtgcccggcatggacccggaactcgccctggccgcactggagtccgcgctcggccgggacgagaccgcgatcaccgtcgcggacatcgactgggaccgcttctacctcgcgtactcctccggtcgcccgcagcccctcgtcgaggagctgcccgaggtgcggcgcatcatcgacgcacgggacagcgccacgtccggacagggcgggagctccgcccagggcgccaaccccctggccgagcggctggccgccgcggctcccggcgagcgtacggagatcctcctcggtctcgtacgggcgcaggccgccgccgtgctccggatgcgttcgccggaggacgtcgccgccgaccgcgccttcaaggacatcggcttcgactcgctcgccggtgtcgagctgcgcaacaggctgacccgggcgaccgggctccagctgcccgcgacgctcgtcttcgaccacccgacgccgctggccctcgtgtcgctgctccgcagcgagttcctcggtgacgaggagacggcggacgcccggcggtccgcggcgctgcccgcgactgtcggtgccggtgccggcgccggcgccggcaccgatgccgacgacgatccgatcgcgatcgtcgcgatgagctgccgctaccccggtgacatccgcagcccggaggacctgtggcggatgctgtccgagggcggcgagggcatcacgccgttccccaccgaccgcggctgggacctcgacggcctgtacgacgccgacccggacgcgctcggcagggcgtacgtccgcgagggcgggttcctgcacgacgcggccgagttcgacgcggagttcttcggcgtctcgccgcgcgaggcgctggccatggacccgcagcagcggatgctcctgacgacgtcctgggaggccttcgagcgggccggcatcgagccggcatcgctgcgcggcagcagcaccggtgtcttcatcggcctctcctaccaggactacgcggcccgcgtcccgaacgccccgcgtggcgtggagggttacctgctgaccggcagcacgccgagcgtcgcgtcgggccgtatcgcgtacaccttcggtctcgaagggcccgcgacgaccgtcgacaccgcctgctcgtcgtcgctgaccgccctgcacctggcggtgcgggcgctgcgcagcggcgagtgcacgatggcgctcgccggtggcgtggcgatgatggcgaccccgcacatgttcgtggagttcagccgtcagcgggcgctcgccccggacggccgcagcaaggccttctcggcggacgccgacgggttcggcgccgcggagggcgtcggcctgctgctcgtggagcggctctcggacgcgcggcgcaacggtcacccggtgctcgccgtggtccgcggtaccgccgtcaaccaggacggcgccagcaacgggctgaccgcgcccaacggaccctcgcagcagcgggtgatccggcaggcgctcgccgacgcccggctggcacccggcgacatcgacgccgtcgagacgcacggcacgggaacctcgctgggcgaccccatcgaggcccagggcctccaggccacgtacggcaaggagcggcccgcggaacggccgctcgccatcggctccgtgaagtccaacatcggacacacccaggccgcggccggtgcggcgggcatcatcaagatggtcctcgcgatgcgccacggcaccctgccgaagaccctccacgccgacgagccgagcccgcacgtcgactgggcgaacagcggcctggccctcgtcaccgagccgatcgactggccggccggcaccggtccgcgccgcgccgccgtctcctccttcggcatcagcgggacgaacgcgcacgtcgtgctggagcaggcgccggatgctgctggtgaggtgcttggggccgatgaggtgcctgaggtgtctgagacggtagcgatggctgggacggctgggacctccgaggtcgctgagggctctgaggcctccgaggcccccgcggcccccggcagccgtgaggcgtccctccccgggcacctgccctgggtgctgtccgccaaggacgagcagtcgctgcgcggccaggccgccgccctgcacgcgtggctgtccgagcccgccgccgacctgtcggacgcggacggaccggcccgcctgcgggacgtcgggtacacgctcgccacgagccgtaccgccttcgcgcaccgcgccgccgtgaccgccgccgaccgggacgggttcctggacgggctggccacgctggcccagggcggcacctcggcccacgtccacctggacaccgcccgggacggcaccaccgcgttcctcttcaccggccagggcagtcagcgccccggcgccggccgtgagctgtacgaccggcaccccgtcttcgcccgggcgctcgacgagatctgcgcccacctcgacggtcacctcgaactgcccctgctcgacgtgatgttcgcggccgagggcagcgcggaggccgcgctgctcgacgagacgcggtacacgcagtgcgcgctgttcgccctggaggtcgcgctcttccggctcgtcgagagctggggcatgcggccggccgcactgctcggtcactcggtcggcgagatcgccgccgcgcacgtcgccggtgtgttctcgctcgccgacgccgcccgcctggtcgccgcgcgcggccggctcatgcaggagctgcccgccggtggcgcgatgctcgccgtccaggccgcggaggacgagatccgcgtgtggctggagacggaggagcggtacgcgggacgtctggacgtcgccgccgtcaacggccccgaggccgccgtcctgtccggcgacgcggacgcggcgcgggaggcggaggcgtactggtccgggctcggccgcaggacccgcgcgctgcgggtcagccacgccttccactccgcgcacatggacggcatgctcgacgggttccgcgccgtcctggagacggtggagttccggcgcccctccctgaccgtggtctcgaacgtcaccggcctggccgccggcccggacgacctgtgcgaccccgagtactgggtccggcacgtccgcggcaccgtccgcttcctcgacggcgtccgtgtcctgcgcgacctcggcgtgcggacctgcctggagctgggccccgacggggtcctcaccgccatggcggccgacggcctcgcggacacccccgcggattccgctgccggctcccccgtcggctctcccgccggctctcccgccgactccgccgccggcgcgctccggccccggccgctgctcgtggcgctgctgcgccgcaagcggtcggagaccgagaccgtcgcggacgccctcggcagggcgcacgcccacggcaccggacccgactggcacgcctggttcgccggctccggggcgcaccgcgtggacctgcccacgtactccttccggcgcgaccgctactggctggacgccccggcggccgacaccgcggtggacaccgccggcctcggtctcggcaccgccgaccacccgctgctcggcgccgtggtcagccttccggaccgggacggcctgctgctcaccggccgcctctccctgcgcacccacccgtggctcgcggaccacgccgtcctggggagcgtcctgctccccggcgccgcgatggtcgaactcgccgcgcacgctgcggagtccgccggtctgcgtgacgtgcgggagctgaccctccttgaaccgctggtactgcccgagcacggtggcgtcgagctgcgcgtgacggtcggggcgccggccggagagcccggtggcgagtcggccggggacggcgcacggcccgtctccctccactcgcggctcgccgacgcgcccgccggtaccgcctggtcctgccacgcgaccggtctgctggccaccgaccggcccgagcttcccgtcgcgcccgaccgtgcggccatgtggccgccgcagggcgccgaggaggtgccgctcgacggtctctacgagcggctcgacgggaacggcctcgccttcggtccgctgttccaggggctgaacgcggtgtggcggtacgagggtgaggtcttcgccgacatcgcgctccccgccaccacgaatgcgaccgcgcccgcgaccgcgaacggcggcgggagtgcggcggcggccccctacggcatccaccccgccctgctcgacgcttcgctgcacgccatcgcggtcggcggtctcgtcgacgagcccgagctcgtccgcgtccccttccactggagcggtgtcaccgtgcacgcggccggtgccgcggcggcccgggtccgtctcgcctccgcggggacggacgccgtctcgctgtccctgacggacggcgagggacgcccgctggtctccgtggaacggctcacgctgcgcccggtcaccgccgatcaggcggcggcgagccgcgtcggcgggctgatgcaccgggtggcctggcgtccgtacgccctcgcctcgtccggcgaacaggacccgcacgccacttcgtacgggccgaccgccgtcctcggcaaggacgagctgaaggtcgccgccgccctggagtccgcgggcgtcgaagtcgggctctaccccgacctggccgcgctgtcccaggacgtggcggccggcgccccggcgccccgtaccgtccttgcgccgctgcccgcgggtcccgccgacggcggcgcggagggtgtacggggcacggtggcccggacgctggagctgctccaggcctggctggccgacgagcacctcgcgggcacccgcctgctcctggtcacccgcggtgcggtgcgggaccccgaggggtccggcgccgacgatggcggcgaggacctgtcgcacgcggccgcctggggtctcgtacggaccgcgcagaccgagaaccccggccgcttcggccttctcgacctggccgacgacgcctcgtcgtaccggaccctgccgtcggtgctctccgacgcgggcctgcgcgacgaaccgcagctcgccctgcacgacggcaccatcaggctggcccgcctggcctccgtccggcccgagaccggcaccgccgcaccggcgctcgccccggagggcacggtcctgctgaccggcggcaccggcggcctgggcggactggtcgcccggcacgtggtgggcgagtggggcgtacgacgcctgctgctggtgagccggcggggcacggacgccccgggcgccgacgagctcgtgcacgagctggaggccctgggagccgacgtctcggtggccgcgtgcgacgtcgccgaccgcgaagccctcaccgccgtactcgacgccatccccgccgaacacccgctcaccgcggtcgtccacacggcaggcgtcctctccgacggcaccctcccgtccatgacgacggaggacgtggaacacgtactgcggcccaaggtcgacgccgcgttcctcctcgacgaactcacctcgacgcccgcatacgacctggcagcgttcgtcatgttctcctccgccgccgccgtcttcggtggcgcggggcagggcgcctacgccgccgccaacgccaccctcgacgccctcgcctggcgccgccgggcagccggactccccgccctctccctcggctggggcctctgggccgagaccagcggcatgaccggcgagctcggccaggcggacctgcgccggatgagccgcgcgggcatcggcgggatcagcgacgccgagggcatcgcgctcctcgacgccgccctccgcgacgaccgccacccggtcctgctgcccctgcggctcgacgccgccgggctgcgggacgcggccgggaacgacccggccggaatcccggcgctcttccgggacgtcgtcggcgccaggaccgtccgggcccggccgtccgcggcctccgcctcgacgacagccgggacggccggcacgccggggacggcggacggcgcggcggaaacggcggcggtcacgctcgccgaccgggccgccaccgtggacgggcccgcacggcagcgcctgctgctcgagttcgtcgtcggcgaggtcgccgaagtactcggccacgcccgcggtcaccggatcgacgccgaacggggcttcctcgacctcggcttcgactccctgaccgccgtcgaactccgcaaccggctcaactccgccggtggcctcgccctcccggcgaccctggtcttcgaccacccaagcccggcggcactcgcctcccacctggacgccgagctgccgcgcggcgcctcggaccaggacggagccgggaaccggaacgggaacgagaacgggacgacggcgtcccggagcaccgccgagacggacgcgctgctggcacaactgacccgcctggaaggcgccttggtgctgacgggcctctcggacgcccccgggagcgaagaagtcctggagcacctgcggtccctgcgctcgatggtcacgggcgagaccgggaccgggaccgcgtccggagccccggacggcgccgggtccggcgccgaggaccggccctgggcggccggggacggagccgggggcgggagtgaggacggcgcgggagtgccggacttcatgaacgcctcggccgaggaactcttcggcctcctcgaccaggaccccagcacggactga

SEQ ID NO: 31 - protein, 4613 aa, Streptomyces venezuelae
MSSAGITRTGARTPVTGRGAAAWDTGEVRVRRGLPPAGPDHAEHSFSRAPTGDVRAELIRGEMSTVSKSESEEFVSVSNDAGSAHGTAEPVAVVGISCRVPGARDPREFWELLAAGGQAVTDVPADRWNAGDFYDPDRSAPGRSNSRWGGFIEDVDRFDAAFFGISPREAAEMDPQQRLALELGWEALERAGIDPSSLTGTRTGVFAGAIWDDYATLKHRQGGAAITPHTVTGLHRGIIANRLSYTLGLRGPSMVVDSGQSSSLVAVHLACESLRRGESELALAGGVSLNLVPDSIIGASKFGGLSPDGRAYTFDARANGYVRGEGGGFVVLKRLSRAVADGDPVLAVIRGSAVNNGGAAQGMTTPDAQAQEAVLREAHERAGTAPADVRYVELHGTGTPVGDPIEAAALGAALGTGRPAGQPLLVGSVKTNIGHLEGAAGIAGLIKAVLAVRGRALPASLNYETPNPAIPFEELNLRVNTEYLPWEPEHDGQRMVVGVSSFGMGGTNAHVVLEEAPGGCRGASVVESTVGGSAVGGGVVPWVVSAKSAAALDAQIERLAAFASRDRTDGVDAGAVDAGAVDAGAVARVLAGGRAQFEHRAVVVGSGPDDLAAALAAPEGLVRGVASGVGRVAFVFPGQGTQWAGMGAELLDSSAVFAAAMAECEAALSPYVDWSLEAVVRQAPGAPTLERVDVVQPVTFAVMVSLARVWQHHGVTPQAVVGHSQGEIAAAYVAGALSLDDAARVVTLRSKSIAAHLAGKGGMLSLALSEDAVLERLAGFDGLSVAAVNGPTATVVSGDPVQIEELARACEADGVRARVIPVDYASHSRQVEIIESELAEVLAGLSPQAPRVPFFSTLEGAWITEPVLDGGYWYRNLRHRVGFAPAVETLATDEGFTHFVEVSAHPVLTMALPGTVTGLATLRRDNGGQDRLVASLAEAWANGLAVDWSPLLPSATGHHSDLPTYAFQTERHWLGEIEALAPAGEPAVQPAVLRTEAAEPAELDRDEQLRVILDKVRAQTAQVLGYATGGQIEVDRTFREAGCTSLTGVDLRNRINAAFGVRMAPSMIFDFPTPEALAEQLLLVVHGEAAANPAGAEPAPVAAAGAVDEPVAIVGMACRLPGGVASPEDLWRLVAGGGDAISEFPQDRGWDVEGLYHPDPEHPGTSYVRQGGFIENVAGFDAAFFGISPREALAMDPQQRLLLETSWEAVEDAGIDPTSLRGRQVGVFTGAMTHEYGPSLRDGGEGLDGYLLTGNTASVMSGRVSYTLGLEGPALTVDTACSSSLVALHLAVQALRKGEVDMALAGGVAVMPTPGMFVEFSRQRGLAGDGRSKAFAASADGTSWSEGVGVLLVERLSDARRNGHQVLAVVRGSALNQDGASNGLTAPNGPSQQRVIRRALADARLTTSDVDVVEAHGTGTRLGDPIEAQALIATYGQGRDDEQPLRLGSLKSNIGHTQAAAGVSGVIKMVQAMRHGLLPKTLHVDEPSDQIDWSAGAVELLTEAVDWPEKQDGGLRRAAVSSFGISGTNAHVVLEEAPVVVEGASVVEPSVGGSAVGGGVTPWVVSAKSAAALDAQIERLAAFASRDRTDDADAGAVDAGAVAHVLADGRAQFEHRAVALGAGADDLVQALADPDGLIRGTASGVGRVAFVFPGQGTQWAGMGAELLDSSAVFAAAMAECEAALSPYVDWSLEAVVRQAPGAPTLERVDVVQPVTFAVMVSLARVWQHHGVTPQAVVGHSQGEIAAAYVAGALPLDDAARVVTLRSKSIAAHLAGKGGMLSLALNEDAVLERLSDFDGLSVAAVNGPTATVVSGDPVQIEELAQACKADGFRARIIPVDYASHSRQVEIIESELAQVLAGLSPQAPRVPFFSTLEGTWITEPVLDGTYWYRNLRHRVGFAPAIETLAVDEGFTHFVEVSAHPVLTMTLPETVTGLGTLRREQGGQERLVTSLAEAWVNGLPVAWTSLLPATASRPGLPTYAFQAERYWLENTPAALATGDDWRYRIDWKRLPAAEGSERTGLSGRWLAVTPEDHSAQAAAVLTALVDAGAKVEVLTAGADDDREALAARLTALTTGDGFTGVVSLLDGLVPQVAWVQALGDAGIKAPLWSVTQGAVSVGRLDTPADPDRAMLWGLGRVVALEHPERWAGLVDLPAQPDAAALAHLVTALSGATGEDQIAIRTTGLHARRLARAPLHGRRPTRDWQPHGTVLITGGTGALGSHAARWMAHHGAEHLLLVSRSGEQAPGATQLTAELTASGARVTIAACDVADPHAMRTLLDAIPAETPLTAVVHTAGALDDGIVDTLTAEQVRRAHRAKAVGASVLDELTRDLDLDAFVLFSSVSSTLGIPGQGNYAPHNAYLDALAARRRATGRSAVSVAWGPWDGGGMAAGDGVAERLRNHGVPGMDPELALAALESALGRDETAITVADIDWDRFYLAYSSGRPQPLVEELPEVRRIIDARDSATSGQGGSSAQGANPLAERLAAAAPGERTEILLGLVRAQAAAVLRMRSPEDVAADRAFKDIGFDSLAGVELRNRLTRATGLQLPATLVFDHPTPLALVSLLRSEFLGDEETADARRSAALPATVGAGAGAGAGTDADDDPIAIVAMSCRYPGDIRSPEDLWRMLSEGGEGITPFPTDRGWDLDGLYDADPDALGRAYVREGGFLHDAAEFDAEFFGVSPREALAMDPQQRMLLTTSWEAFERAGIEPASLRGSSTGVFIGLSYQDYAARVPNAPRGVEGYLLTGSTPSVASGRIAYTFGLEGPATTVDTACSSSLTALHLAVRALRSGECTMALAGGVAMMATPHMFVEFSRQRALAPDGRSKAFSADADGFGAAEGVGLLLVERLSDARRNGHPVLAVVRGTAVNQDGASNGLTAPNGPSQQRVIRQALADARLAPGDIDAVETHGTGTSLGDPIEAQGLQATYGKERPAERPLAIGSVKSNIGHTQAAAGAAGIIKMVLAMRHGTLPKTLHADEPSPHVDWANSGLALVTEPIDWPAGTGPRRAAVSSFGISGTNAHVVLEQAPDAAGEVLGADEVPEVSETVAMAGTAGTSEVAEGSEASEAPAAPGSREASLPGHLPWVLSAKDEQSLRGQAAALHAWLSEPAADLSDADGPARLRDVGYTLATSRTAFAHRAAVTAADRDGFLDGLATLAQGGTSAHVHLDTARDGTTAFLFTGQGSQRPGAGRELYDRHPVFARALDEICAHLDGHLELPLLDVMFAAEGSAEAALLDETRYTQCALFALEVALFRLVESWGMRPAALLGHSVGEIAAAHVAGVFSLADAARLVAARGRLMQELPAGGAMLAVQAAEDEIRVWLETEERYAGRLDVAAVNGPEAAVLSGDADAAREAEAYWSGLGRRTRALRVSHAFHSAHMDGMLDGFRAVLETVEFRRPSLTVVSNVTGLAAGPDDLCDPEYWVRHVRGTVRFLDGVRVLRDLGVRTCLELGPDGVLTAMAADGLADTPADSAAGSPVGSPAGSPADSAAGALRPRPLLVALLRRKRSETETVADALGRAHAHGTGPDWHAWFAGSGAHRVDLPTYSFRRDRYWLDAPAADTAVDTAGLGLGTADHPLLGAVVSLPDRDGLLLTGRLSLRTHPWLADHAVLGSVLLPGAAMVELAAHAAESAGLRDVRELTLLEPLVLPEHGGVELRVTVGAPAGEPGGESAGDGARPVSLHSRLADAPAGTAWSCHATGLLATDRPELPVAPDRAAMWPPQGAEEVPLDGLYERLDGNGLAFGPLFQGLNAVWRYEGEVFADIALPATTNATAPATANGGGSAAAAPYGIHPALLDASLHAIAVGGLVDEPELVRVPFHWSGVTVHAAGAAAARVRLASAGTDAVSLSLTDGEGRPLVSVERLTLRPVTADQAAASRVGGLMHRVAWRPYALASSGEQDPHATSYGPTAVLGKDELKVAAALESAGVEVGLYPDLAALSQDVAAGAPAPRTVLAPLPAGPADGGAEGVRGTVARTLELLQAWLADEHLAGTRLLLVTRGAVRDPEGSGADDGGEDLSHAAAWGLVRTAQTENPGRFGLLDLADDASSYRTLPSVLSDAGLRDEPQLALHDGTIRLARLASVRPETGTAAPALAPEGTVLLTGGTGGLGGLVARHVVGEWGVRRLLLVSRRGTDAPGADELVHELEALGADVSVAACDVADREALTAVLDAIPAEHPLTAVVHTAGVLSDGTLPSMTTEDVEHVLRPKVDAAFLLDELTSTPAYDLAAFVMFSSAAAVFGGAGQGAYAAANATLDALAWRRRAAGLPALSLGWGLWAETSGMTGELGQADLRRMSRAGIGGISDAEGIALLDAALRDDRHPVLLPLRLDAAGLRDAAGNDPAGIPALFRDVVGARTVRARPSAASASTTAGTAGTPGTADGAAETAAVTLADRAATVDGPARQRLLLEFVVGEVAEVLGHARGHRIDAERGFLDLGFDSLTAVELRNRLNSAGGLALPATLVFDHPSPAALASHLDAELPRGASDQDGAGNRNGNENGTTASRSTAETDALLAQLTRLEGALVLTGLSDAPGSEEVLEHLRSLRSMVTGETGTGTASGAPDGAGSGAEDRPWAAGDGAGGGSEDGAGVPDFMNASAEELFGLLDQDPSTD

SEQ ID NO: 32 - DNA, 11220 bp, Streptomyces venezuelae
gtgtccacggtgaacgaagagaagtacctcgactacctgcgtcgtgccacggcggacctccacgaggcccgtggccgcctccgcgagctggaggcgaaggcgggcgagccggtggcgatcgtcggcatggcctgccgcctgcccggcggcgtcgcctcgcccgaggacctgtggcggctggtggccggcggcgaggacgcgatctcggagttcccccaggaccgcggctgggacgtggagggcctgtacgacccgaacccggaggccacgggcaagagttacgcccgcgaggccggattcctgtacgaggcgggcgagttcgacgccgacttcttcgggatctcgccgcgcgaggccctcgccatggacccgcagcagcgtctcctcctggaggcctcctgggaggcgttcgagcacgccgggatcccggcggccaccgcgcgcggcacctcggtcggcgtcttcaccggcgtgatgtaccacgactacgccacccgtctcaccgatgtcccggagggcatcgagggctacctgggcaccggcaactccggcagtgtcgcctcgggccgcgtcgcgtacacgcttggcctggaggggccggccgtcacggtcgacaccgcctgctcgtcctcgctggtcgccctgcacctcgccgtgcaggccctgcgcaagggcgaggtcgacatggcgctcgccggcggcgtgacggtcatgtcgacgcccagcaccttcgtcgagttcagccgtcagcgcgggctggcgccggacggccggtcgaagtccttctcgtcgacggccgacggcaccagctggtccgagggcgtcggcgtcctcctcgtcgagcgcctgtccgacgcgcgtcgcaagggccatcggatcctcgccgtggtccggggcaccgccgtcaaccaggacggcgccagcagcggcctcacggctccgaacgggccgtcgcagcagcgcgtcatccgacgtgccctggcggacgcccggctcacgacctccgacgtggacgtcgtcgaggcccacggcacgggtacgcgactcggcgacccgatcgaggcgcaggccgtcatcgccacgtacgggcagggccgtgacggcgaacagccgctgcgcctcgggtcgttgaagtccaacatcggacacacccaggccgccgccggtgtctccggcgtgatcaagatggtccaggcgatgcgccacggcgtcctgccgaagacgctccacgtggagaagccgacggaccaggtggactggtccgcgggcgcggtcgagctgctcaccgaggccatggactggccggacaagggcgacggcggactgcgcagggccgcggtctcctccttcggcgtcagcgggacgaacgcgcacgtcgtgctcgaagaggccccggcggccgaggagacccctgcctccgaggcgaccccggccgtcgagccgtcggtcggcgccggcctggtgccgtggctggtgtcggcgaagactccggccgcgctggacgcccagatcggacgcctcgccgcgttcgcctcgcagggccgtacggacgccgccgatccgggcgcggtcgctcgcgtactggccggcgggcgcgccgagttcgagcaccgggccgtcgtgctcggcaccggacaggacgatttcgcgcaggcgctgaccgctccggaaggactgatacgcggcacgccctcggacgtgggccgggtggcgttcgtgttccccggtcagggcacgcagtgggccgggatgggcgccgaactcctcgacgtgtcgaaggagttcgcggcggccatggccgagtgcgagagcgcgctctcccgctatgtcgactggtcgctggaggccgtcgtccggcaggcgccgggcgcgcccacgctggagcgggtcgacgtcgtccagcccgtgaccttcgctgtcatggtttcgctggcgaaggtctggcagcaccacggcgtgacgccgcaggccgtcgtcggccactcgcagggcgagatcgccgccgcgtacgtcgccggtgccctcaccctcgacgacgccgcccgcgtcgtcaccctgcgcagcaagtccatcgccgcccacctcgccggcaagggcggcatgatctccctcgccctcagcgaggaagccacccggcagcgcatcgagaacctccacggactgtcgatcgccgccgtcaacggccccaccgccaccgtggtttcgggcgaccccacccagatccaagagctcgctcaggcgtgtgaggccgacggggtccgcgcacggatcatccccgtcgactacgcctcccacagcgcccacgtcgagaccatcgagagcgaactcgccgaggtcctcgccgggctcagcccgcggacacctgaggtgccgttcttctcgacactcgaaggcgcctggatcaccgagccggtgctcgacggcacctactggtaccgcaacctccgccaccgcgtcggcttcgcccccgccgtcgagaccctcgccaccgacgaaggcttcacccacttcatcgaggtcagcgcccaccccgtcctcaccatgaccctccccgagaccgtcaccggcctcggcaccctccgccgcgaacagggaggccaggagcgtctggtcacctcactcgccgaagcctggaccaacggcctcaccatcgactgggcgcccgtcctccccaccgcaaccggccaccaccccgagctccccacctacgccttccagcgccgtcactactggctccacgactcccccgccgtccagggctccgtgcaggactcctggcgctaccgcatcgactggaagcgcctcgcggtcgccgacgcgtccgagcgcgccgggctgtccgggcgctggctcgtcgtcgtccccgaggaccgttccgccgaggccgccccggtgctcgccgcgctgtccggcgccggcgccgaccccgtacagctggacgtgtccccgctgggcgaccggcagcggctcgccgcgacgctgggcgaggccctggcggcggccggtggagccgtcgacggcgtcctctcgctgctcgcgtgggacgagagcgcgcaccccggccaccccgcccccttcacccggggcaccggcgccaccctcaccctggtgcaggcgctggaggacgccggcgtcgccgccccgctgtggtgcgtgacccacggcgcggtgtccgtcggccgggccgaccacgtcacctcccccgcccaggccatggtgtggggcatgggccgggtcgccgccctggagcaccccgagcggtggggcggcctgatcgacctgccctcggacgccgaccgggcggccctggaccgcatgaccacggtcctcgccggcggtacgggtgaggaccaggtcgcggtacgcgcctccgggctgctcgcccgccgcctcgtccgcgcctccctcccggcgcacggcacggcttcgccgtggtggcaggccgacggcacggtgctcgtcaccggtgccgaggagcctgcggccgccgaggccgcacgccggctggcccgcgacggcgccggacacctcctcctccacaccaccccctccggcagcgaaggcgccgaaggcacctccggtgccgccgaggactccggcctcgccgggctcgtcgccgaactcgcggacctgggcgcgacggccaccgtcgtgacctgcgacctcacggacgcggaggcggccgcccggctgctcgccggcgtctccgacgcgcacccgctcagcgccgtcctccacctgccgcccaccgtcgactccgagccgctcgccgcgaccgacgcggacgcgctcgcccgtgtcgtgaccgcgaaggccaccgccgcgctccacctggaccgcctcctgcgggaggccgcggctgccggaggccgtccgcccgtcctggtcctcttctcctcggtcgccgcgatctggggcggcgccggtcagggcgcgtacgccgccggtacggccttcctcgacgccctcgccggtcagcaccgggccgacggccccaccgtgacctcggtggcctggagcccctgggagggcagccgcgtcaccgagggtgcgaccggggagcggctgcgccgcctcggcctgcgccccctcgcccccgcgacggcgctcaccgccctggacaccgcgctcggccacggcgacaccgccgtcacgatcgccgacgtcgactggtcgagcttcgcccccggcttcaccacggcccggccgggcaccctcctcgccgatctgcccgaggcgcgccgcgcgctcgacgagcagcagtcgacgacggccgccgacgacaccgtcctgagccgcgagctcggtgcgctcaccggcgccgaacagcagcgccgtatgcaggagttggtccgcgagcacctcgccgtggtcctcaaccacccctcccccgaggccgtcgacacggggcgggccttccgtgacctcggattcgactcgctgacggcggtcgagctccgcaaccgcctcaagaacgccaccggcctggccctcccggccactctggtcttcgactacccgaccccccggacgctggcggagttcctcctcgcggagatcctgggcgagcaggccggtgccggcgagcagcttccggtggacggcggggtcgacgacgagcccgtcgcgatcgtcggcatggcgtgccgcctgccgggcggtgtcgcctcgccggaggacctgtggcggctggtggccggcggcgaggacgcgatctccggcttcccgcaggaccgcggctgggacgtggaggggctgtacgacccggacccggacgcgtccgggcggacgtactgccgtgccggtggcttcctcgacgaggcgggcgagttcgacgccgacttcttcgggatctcgccgcgcgaggccctcgccatggacccgcagcagcggctcctcctggagacctcctgggaggccgtcgaggacgccgggatcgacccgacctcccttcaggggcagcaggtcggcgtgttcgcgggcaccaacggcccccactacgagccgctgctccgcaacaccgccgaggatcttgagggttacgtcgggacgggcaacgccgccagcatcatgtcgggccgtgtctcgtacaccctcggcctggagggcccggccgtcacggtcgacaccgcctgctcctcctcgctggtcgccctgcacctcgccgtgcaggccctgcgcaagggcgaatgcggactggcgctcgcgggcggtgtgacggtcatgtcgacgcccacgacgttcgtggagttcagccggcagcgcgggctcgcggaggacggccggtcgaaggcgttcgccgcgtcggcggacggcttcggcccggcggagggcgtcggcatgctcctcgtcgagcgcctgtcggacgcccgccgcaacggacaccgtgtgctggcggtcgtgcgcggcagcgcggtcaaccaggacggcgcgagcaacggcctgaccgccccgaacgggccctcgcagcagcgcgtcatccggcgcgcgctcgcggacgcccgactgacgaccgccgacgtggacgtcgtcgaggcccacggcacgggcacgcgactcggcgacccgatcgaggcacaggccctcatcgccacctacggccaggggcgcgacaccgaacagccgctgcgcctggggtcgttgaagtccaacatcggacacacccaggccgccgccggtgtctccggcatcatcaagatggtccaggcgatgcgccacggcgtcctgccgaagacgctccacgtggaccggccgtcggaccagatcgactggtcggcgggcacggtcgagctgctcaccgaggccatggactggccgaggaagcaggagggcgggctgcgccgcgcggccgtctcctccttcggcatcagcggcacgaacgcgcacatcgtgctcgaagaagccccggtcgacgaggacgccccggcggacgagccgtcggtcggcggtgtggtgccgtggctcgtgtccgcgaagactccggccgcgctggacgcccagatcggacgcctcgccgcgttcgcctcgcagggccgtacggacgccgccgatccgggcgcggtcgctcgcgtactggccggcgggcgtgcgcagttcgagcaccgggccgtcgcgctcggcaccggacaggacgacctggcggccgcactggccgcgcctgagggtctggtccggggtgtggcctccggtgtgggtcgagtggcgttcgtgttcccgggacagggcacgcagtgggccgggatgggtgccgaactcctcgacgtgtcgaaggagttcgcggcggccatggccgagtgcgaggccgcgctcgctccgtacgtggactggtcgctggaggccgtcgtccgacaggcccccggcgcgcccacgctggagcgggtcgatgtcgtccagcccgtgacgttcgccgtcatggtctcgctggcgaaggtctggcagcaccacggggtgaccccgcaagccgtcgtcggccactcgcagggcgagatcgccgccgcgtacgtcgccggtgccctgagcctggacgacgccgctcgtgtcgtgaccctgcgcagcaagtccatcggcgcccacctcgcgggccagggcggcatgctgtccctcgcgctgagcgaggcggccgttgtggagcgactggccgggttcgacgggctgtccgtcgccgccgtcaacgggcctaccgccaccgtggtttcgggcgacccgacccagatccaagagctcgctcaggcgtgtgaggccgacggggtccgcgcacggatcatccccgtcgactacgcctcccacagcgcccacgtcgagaccatcgagagcgaactcgccgacgtcctggcggggttgtccccccagacaccccaggtccccttcttctccaccctcgaaggcgcctggatcaccgaacccgccctcgacggcggctactggtaccgcaacctccgccatcgtgtgggcttcgccccggccgtcgaaaccctggccaccgacgaaggcttcacccacttcgtcgaggtcagcgcccaccccgtcctcaccatggcgctgcccgagaccgtcaccggactcggcaccctccgccgtgacaacggcggacagcaccgcctcaccacctccctcgccgaggcctgggccaacggcctcaccgtcgactgggcctctctcctccccaccacgaccacccaccccgatctgcccacctacgccttccagaccgagcgctactggccgcagcccgacctctccgccgccggtgacatcacctccgccggtctcggggcggccgagcacccgctgctcggcgcggccgtggcgctcgcggactccgacggctgcctgctcacggggagcctctccctccgtacgcacccctggctggcggaccacgcggtggccggcaccgtgctgctgccgggaacggcgttcgtggagctggcgttccgagccggggaccaggtcggttgcgatctggtcgaggagctcaccctcgacgcgccgctcgtgctgccccgtcgtggcgcggtccgtgtgcagctgtccgtcggcgcgagcgacgagtccgggcgtcgtaccttcgggctctacgcgcacccggaggacgcgccgggcgaggcggagtggacgcggcacgccaccggtgtgctggccgcccgtgcggaccgcaccgcccccgtcgccgacccggaggcctggccgccgccgggcgccgagccggtggacgtggacggtctgtacgagcgcttcgcggcgaacggctacggctacggccccctcttccagggcgtccgtggtgtctggcggcgtggcgacgaggtgttcgccgacgtggccctgccggccgaggtcgccggtgccgagggcgcgcggttcggccttcacccggcgctgctcgacgccgccgtgcaggcggccggtgcgggccggggcgttcggcgcgggcacgcggctgccgttcgcctggagcgggatctcctgtacgcggtcggcgccaccgccctccgcgtgcggctggcccccgccggcccggacacggtgtccgtgagcgccgccgactcctccgggcagccggtgttcgccgcggactccctcacggtgctgcccgtcgaccccgcgcagctggcggccttcagcgacccgactctggacgcgctgcacctgctggagtggaccgcctgggacggtgccgcgcaggccctgcccggcgcggtcgtgctgggcggcgacgccgacggtctcgccgcggcgctgcgcgccggtggcaccgaggtcctgtccttcccggaccttacggacctggtggaggccgtcgaccggggcgagaccccggccccggcgaccgtcctggtggcctgccccgccgccggccccgatgggccggagcatgtccgcgaggccctgcacgggtcgctcgcgctgatgcaggcctggctggccgacgagcggttcaccgatgggcgcctggtgctcgtgacccgcgacgcggtcgccgcccgttccggcgacggcctgcggtccacgggacaggccgccgtctggggcctcggccggtccgcgcagacggagagcccgggccggttcgtcctgctcgacctcgccggggaagcccggacggccggggacgccaccgccggggacggcctgacgaccggggacgccaccgtcggcggcacctctggagacgccgccctcggcagcgccctcgcgaccgccctcggctcgggcgagccgcagctcgccctccgggacggggcgctcctcgtaccccgcctggcgcgggccgccgcgcccgccgcggccgacggcctcgccgcggccgacggcctcgccgctctgccgctgcccgccgctccggccctctggcgtctggagcccggtacggacggcagcctggagagcctcacggcggcgcccggcgacgccgagaccctcgccccggagccgctcggcccgggacaggtccgcatcgcgatccgggccaccggtctcaacttccgcgacgtcctgatcgccctcggcatgtaccccgatccggcgctgatgggcaccgagggagccggcgtggtcaccgcgaccggccccggcgtcacgcacctcgcccccggcgaccgggtcatgggcctgctctccggcgcgtacgccccggtcgtcgtggcggacgcgcggaccgtcgcgcggatgcccgaggggtggacgttcgcccagggcgcctccgtgccggtggtgttcctgacggccgtctacgccctgcgcgacctggcggacgtcaagcccggcgagcgcctcctggtccactccgccgccggtggcgtgggcatggccgccgtgcagctcgcccggcactggggcgtggaggtccacggcacggcgagtcacgggaagtgggacgccctgcgcgcgctcggcctggacgacgcgcacatcgcctcctcccgcaccctggacttcgagtccgcgttccgtgccgcttccggcggggcgggcatggacgtcgtactgaactcgctcgcccgcgagttcgtcgacgcctcgctgcgcctgctcgggccgggcggccggttcgtggagatggggaagaccgacgtccgcgacgcggagcgggtcgccgccgaccaccccggtgtcggctaccgcgccttcgacctgggcgaggccgggccggagcggatcggcgagatgctcgccgaggtcatcgccctcttcgaggacggggtgctccggcacctgcccgtcacgacctgggacgtgcgccgggcccgcgacgccttccggcacgtcagccaggcccgccacacgggcaaggtcgtcctcacgatgccgtcgggcctcgacccggagggtacggtcctgctgaccggcggcaccggtgcgctggggggcatcgtggcccggcacgtggtgggcgagtggggcgtacgacgcctgctgctcgtgagccggcggggcacggacgccccgggcgccggcgagctcgtgcacgagctggaggccctgggagccgacgtctcggtggccgcgtgcgacgtcgccgaccgcgaagccctcaccgccgtactcgactcgatccccgccgaacacccgctcaccgcggtcgtccacacggcaggcgtcctctccgacggcaccctcccctcgatgacagcggaggatgtggaacacgtactgcgtcccaaggtcgacgccgcgttcctcctcgacgaactcacctcgacgcccggctacgacctggcagcgttcgtcatgttctcctccgccgccgccgtcttcggtggcgcggggcagggcgcctacgccgccgccaacgccaccctcgacgccctcgcctggcgccgccggacagccggactccccgccctctccctcggctggggcctctgggccgagaccagcggcatgaccggcggactcagcgacaccgaccgctcgcggctggcccgttccggggcgacgcccatggacagcgagctgaccctgtccctcctggacgcggccatgcgccgcgacgacccggcgctcgtcccgatcgccctggacgtcgccgcgctccgcgcccagcagcgcgacggcatgctggcgccgctgctcagcgggctcacccgcggatcgcgggtcggcggcgcgccggtcaaccagcgcagggcagccgccggaggcgcgggcgaggcggacacggacctcggcgggcggctcgccgcgatgacaccggacgaccgggtcgcgcacctgcgggacctcgtccgtacgcacgtggcgaccgtcctgggacacggcaccccgagccgggtggacctggagcgggccttccgcgacaccggtttcgactcgctcaccgccgtcgaactccgcaaccgtctcaacgccgcgaccgggctgcggctgccggccacgctggtcttcgaccaccccaccccgggggagctcgccgggcacctgctcgacgaactcgccacggccgcgggcgggtcctgggcggaaggcaccgggtccggagacacggcctcggcgaccgatcggcagaccacggcggccctcgccgaactcgaccggctggaaggcgtgctcgcctccctcgcgcccgccgccggcggccgtccggagctcgccgcccggctcagggcgctggccgcggccctgggggacgacggcgacgacgccaccgacctggacgaggcgtccgacgacgacctcttctccttcatcgacaaggagctgggcgactccgacttctga

SEQ ID NO: 33 - protein, 3739 aa, Streptomyces venezuelae
MSTVNEEKYLDYLRRATADLHEARGRLRELEAKAGEPVAIVGMACRLPGGVASPEDLWRLVAGGEDAISEFPQDRGWDVEGLYDPNPEATGKSYAREAGFLYEAGEFDADFFGISPREALAMDPQQRLLLEASWEAFEHAGIPAATARGTSVGVFTGVMYHDYATRLTDVPEGIEGYLGTGNSGSVASGRVAYTLGLEGPAVTVDTACSSSLVALHLAVQALRKGEVDMALAGGVTVMSTPSTFVEFSRQRGLAPDGRSKSFSSTADGTSWSEGVGVLLVERLSDARRKGHRILAVVRGTAVNQDGASSGLTAPNGPSQQRVIRRALADARLTTSDVDVVEAHGTGTRLGDPIEAQAVIATYGQGRDGEQPLRLGSLKSNIGHTQAAAGVSGVIKMVQAMRHGVLPKTLHVEKPTDQVDWSAGAVELLTEAMDWPDKGDGGLRRAAVSSFGVSGTNAHVVLEEAPAAEETPASEATPAVEPSVGAGLVPWLVSAKTPAALDAQIGRLAAFASQGRTDAADPGAVARVLAGGRAEFEHRAVVLGTGQDDFAQALTAPEGLIRGTPSDVGRVAFVFPGQGTQWAGMGAELLDVSKEFAAAMAECESALSRYVDWSLEAVVRQAPGAPTLERVDVVQPVTFAVMVSLAKVWQHHGVTPQAVVGHSQGEIAAAYVAGALTLDDAARVVTLRSKSIAAHLAGKGGMISLALSEEATRQRIENLHGLSIAAVNGPTATVVSGDPTQIQELAQACEADGVRARIIPVDYASHSAHVETIESELAEVLAGLSPRTPEVPFFSTLEGAWITEPVLDGTYWYRNLRHRVGFAPAVETLATDEGFTHFIEVSAHPVLTMTLPETVTGLGTLRREQGGQERLVTSLAEAWTNGLTIDWAPVLPTATGHHPELPTYAFQRRHYWLHDSPAVQGSVQDSWRYRIDWKRLAVADASERAGLSGRWLVVVPEDRSAEAAPVLAALSGAGADPVQLDVSPLGDRQRLAATLGEALAAAGGAVDGVLSLLAWDESAHPGHPAPFTRGTGATLTLVQALEDAGVAAPLWCVTHGAVSVGRADHVTSPAQAMVWGMGRVAALEHPERWGGLIDLPSDADRAALDRMTTVLAGGTGEDQVAVRASGLLARRLVRASLPAHGTASPWWQADGTVLVTGAEEPAAAEAARRLARDGAGHLLLHTTPSGSEGAEGTSGAAEDSGLAGLVAELADLGATATVVTCDLTDAEAAARLLAGVSDAHPLSAVLHLPPTVDSEPLAATDADALARVVTAKATAALHLDRLLREAAAAGGRPPVLVLFSSVAAIWGGAGQGAYAAGTAFLDALAGQHRADGPTVTSVAWSPWEGSRVTEGATGERLRRLGLRPLAPATALTALDTALGHGDTAVTIADVDWSSFAPGFTTARPGTLLADLPEARRALDEQQSTTAADDTVLSRELGALTGAEQQRRMQELVREHLAVVLNHPSPEAVDTGRAFRDLGFDSLTAVELRNRLKNATGLALPATLVFDYPTPRTLAEFLLAEILGEQAGAGEQLPVDGGVDDEPVAIVGMACRLPGGVASPEDLWRLVAGGEDAISGFPQDRGWDVEGLYDPDPDASGRTYCRAGGFLDEAGEFDADFFGISPREALAMDPQQRLLLETSWEAVEDAGIDPTSLQGQQVGVFAGTNGPHYEPLLRNTAEDLEGYVGTGNAASIMSGRVSYTLGLEGPAVTVDTACSSSLVALHLAVQALRKGECGLALAGGVTVMSTPTTFVEFSRQRGLAEDGRSKAFAASADGFGPAEGVGMLLVERLSDARRNGHRVLAVVRGSAVNQDGASNGLTAPNGPSQQRVIRRALADARLTTADVDVVEAHGTGTRLGDPIEAQALIATYGQGRDTEQPLRLGSLKSNIGHTQAAAGVSGIIKMVQAMRHGVLPKTLHVDRPSDQIDWSAGTVELLTEAMDWPRKQEGGLRRAAVSSFGISGTNAHIVLEEAPVDEDAPADEPSVGGVVPWLVSAKTPAALDAQIGRLAAFASQGRTDAADPGAVARVLAGGRAQFEHRAVALGTGQDDLAAALAAPEGLVRGVASGVGRVAFVFPGQGTQWAGMGAELLDVSKEFAAAMAECEAALAPYVDWSLEAVVRQAPGAPTLERVDVVQPVTFAVMVSLAKVWQHHGVTPQAVVGHSQGEIAAAYVAGALSLDDAARVVTLRSKSIGAHLAGQGGMLSLALSEAAVVERLAGFDGLSVAAVNGPTATVVSGDPTQIQELAQACEADGVRARIIPVDYASHSAHVETIESELADVLAGLSPQTPQVPFFSTLEGAWITEPALDGGYWYRNLRHRVGFAPAVETLATDEGFTHFVEVSAHPVLTMALPETVTGLGTLRRDNGGQHRLTTSLAEAWANGLTVDWASLLPTTTTHPDLPTYAFQTERYWPQPDLSAAGDITSAGLGAAEHPLLGAAVALADSDGCLLTGSLSLRTHPWLADHAVAGTVLLPGTAFVELAFRAGDQVGCDLVEELTLDAPLVLPRRGAVRVQLSVGASDESGRRTFGLYAHPEDAPGEAEWTRHATGVLAARADRTAPVADPEAWPPPGAEPVDVDGLYERFAANGYGYGPLFQGVRGVWRRGDEVFADVALPAEVAGAEGARFGLHPALLDAAVQAAGAGRGVRRGHAAAVRLERDLLYAVGATALRVRLAPAGPDTVSVSAADSSGQPVFAADSLTVLPVDPAQLAAFSDPTLDALHLLEWTAWDGAAQALPGAVVLGGDADGLAAALRAGGTEVLSFPDLTDLVEAVDRGETPAPATVLVACPAAGPDGPEHVREALHGSLALMQAWLADERFTDGRLVLVTRDAVAARSGDGLRSTGQAAVWGLGRSAQTESPGRFVLLDLAGEARTAGDATAGDGLTTGDATVGGTSGDAALGSALATALGSGEPQLALRDGALLVPRLARAAAPAAADGLAAADGLAALPLPAAPALWRLEPGTDGSLESLTAAPGDAETLAPEPLGPGQVRIAIRATGLNFRDVLIALGMYPDPALMGTEGAGVVTATGPGVTHLAPGDRVMGLLSGAYAPVVVADARTVARMPEGWTFAQGASVPVVFLTAVYALRDLADVKPGERLLVHSAAGGVGMAAVQLARHWGVEVHGTASHGKWDALRALGLDDAHIASSRTLDFESAFRAASGGAGMDVVLNSLAREFVDASLRLLGPGGRFVEMGKTDVRDAERVAADHPGVGYRAFDLGEAGPERIGEMLAEVIALFEDGVLRHLPVTTWDVRRARDAFRHVSQARHTGKVVLTMPSGLDPEGTVLLTGGTGALGGIVARHVVGEWGVRRLLLVSRRGTDAPGAGELVHELEALGADVSVAACDVADREALTAVLDSIPAEHPLTAVVHTAGVLSDGTLPSMTAEDVEHVLRPKVDAAFLLDELTSTPGYDLAAFVMFSSAAAVFGGAGQGAYAAANATLDALAWRRRTAGLPALSLGWGLWAETSGMTGGLSDTDRSRLARSGATPMDSELTLSLLDAAMRRDDPALVPIALDVAALRAQQRDGMLAPLLSGLTRGSRVGGAPVNQRRAAAGGAGEADTDLGGRLAAMTPDDRVAHLRDLVRTHVATVLGHGTPSRVDLERAFRDTGFDSLTAVELRNRLNAATGLRLPATLVFDHPTPGELAGHLLDELATAAGGSWAEGTGSGDTASATDRQTTAALAELDRLEGVLASLAPAAGGRPELAARLRALAAALGDDGDDATDLDEASDDDLFSFIDKELGDSDF

SEQ ID NO: 34 - DNA, 4689 bp, Streptomyces venezuelae
atggcgaacaacgaagacaagctccgcgactacctcaagcgcgtcaccgccgagctgcagcagaacaccaggcgtctgcgcgagatcgagggacgcacgcacgagccggtggcgatcgtgggcatggcctgccgcctgccgggcggtgtcgcctcgcccgaggacctgtggcagctggtggccggggacggggacgcgatctcggagttcccgcaggaccgcggctgggacgtggaggggctgtacgaccccgacccggacgcgtccggcaggacgtactgccggtccggcggattcctgcacgacgccggcgagttcgacgccgacttcttcgggatctcgccgcgcgaggccctcgccatggacccgcagcagcgactgtccctcaccaccgcgtgggaggcgatcgagagcgcgggcatcgacccgacggccctgaagggcagcggcctcggcgtcttcgtcggcggctggcacaccggctacacctcggggcagaccaccgccgtgcagtcgcccgagctggagggccacctggtcagcggcgcggcgctgggcttcctgtccggccgtatcgcgtacgtcctcggtacggacggaccggccctgaccgtggacacggcctgctcgtcctcgctggtcgccctgcacctcgccgtgcaggccctccgcaagggcgagtgcgacatggccctcgccggtggtgtcacggtcatgcccaacgcggacctgttcgtgcagttcagccggcagcgcgggctggccgcggacggccggtcgaaggcgttcgccacctcggcggacggcttcggccccgcggagggcgccggagtcctgctggtggagcgcctgtcggacgcccgccgcaacggacaccggatcctcgcggtcgtccgcggcagcgcggtcaaccaggacggcgccagcaacggcctcacggctccgcacgggccctcccagcagcgcgtcatccgacgggccctggcggacgcccggctcgcgccgggtgacgtggacgtcgtcgaggcgcacggcacgggcacgcggctcggcgacccgatcgaggcgcaggccctcatcgccacctacggccaggagaagagcagcgaacagccgctgaggctgggcgcgttgaagtcgaacatcgggcacacgcaggccgcggccggtgtcgcaggtgtcatcaagatggtccaggcgatgcgccacggactgctgccgaagacgctgcacgtcgacgagccctcggaccagatcgactggtcggcgggcacggtggaactcctcaccgaggccgtcgactggccggagaagcaggacggcgggctgcgccgcgcggctgtctcctccttcggcatcagcgggacgaacgcgcacgtcgtcctggaggaggccccggcggtcgaggactccccggccgtcgagccgccggccggtggcggtgtggtgccgtggccggtgtccgcgaagactccggccgcgctggacgcccagatcgggcagctcgccgcgtacgcggacggtcgtacggacgtggatccggcggtggccgcccgcgccctggtcgacagccgtacggcgatggagcaccgcgcggtcgcggtcggcgacagccgggaggcactgcgggacgccctgcggatgccggaaggactggtacgcggcacgtcctcggacgtgggccgggtggcgttcgtcttccccggccagggcacgcagtgggccggcatgggcgccgaactccttgacagctcaccggagttcgctgcctcgatggccgaatgcgagaccgcgctctcccgctacgtcgactggtctcttgaagccgtcgtccgacaggaacccggcgcacccacgctcgaccgcgtcgacgtcgtccagcccgtgaccttcgctgtcatggtctcgctggcgaaggtctggcagcaccacggcatcaccccccaggccgtcgtcggccactcgcagggcgagatcgccgccgcgtacgtcgccggtgcactcaccctcgacgacgccgcccgcgtcgtcaccctgcgcagcaagtccatcgccgcccacctcgccggcaagggcggcatgatctccctcgccctcgacgaggcggccgtcctgaagcgactgagcgacttcgacggactctccgtcgccgccgtcaacggccccaccgccaccgtcgtctccggcgacccgacccagatcgaggaactcgcccgcacctgcgaggccgacggcgtccgtgcgcggatcatcccggtcgactacgcctcccacagccggcaggtcgagatcatcgagaaggagctggccgaggtcctcgccggactcgccccgcaggctccgcacgtgccgttcttctccaccctcgaaggcacctggatcaccgagccggtgctcgacggcacctactggtaccgcaacctgcgccatcgcgtgggcttcgcccccgccgtggagaccttggcggttgacggcttcacccacttcatcgaggtcagcgcccaccccgtcctcaccatgaccctccccgagaccgtcaccggcctcggcaccctccgccgcgaacagggaggccaggagcgtctggtcacctcactcgccgaagcctgggccaacggcctcaccatcgactgggcgcccatcctccccaccgcaaccggccaccaccccgagctccccacctacgccttccagaccgagcgcttctggctgcagagctccgcgcccaccagcgccgccgacgactggcgttaccgcgtcgagtggaagccgctgacggcctccggccaggcggacctgtccgggcggtggatcgtcgccgtcgggagcgagccagaagccgagctgctgggcgcgctgaaggccgcgggagcggaggtcgacgtactggaagccggggcggacgacgaccgtgaggccctcgccgcccggctcaccgcactgacgaccggcgacggcttcaccggcgtggtctcgctcctcgacgacctcgtgccacaggtcgcctgggtgcaggcactcggcgacgccggaatcaaggcgcccctgtggtccgtcacccagggcgcggtctccgtcggacgtctcgacacccccgccgaccccgaccgggccatgctctggggcctcggccgcgtcgtcgcccttgagcaccccgaacgctgggccggcctcgtcgacctccccgcccagcccgatgccgccgccctcgcccacctcgtcaccgcactctccggcgccaccggcgaggaccagatcgccatccgcaccaccggactccacgcccgccgcctcgcccgcgcacccctccacggacgtcggcccacccgcgactggcagccccacggcaccgtcctcatcaccggcggcaccggagccctcggcagccacgccgcacgctggatggcccaccacggagccgaacacctcctcctcgtcagccgcagcggcgaacaagcccccggagccacccaactcaccgccgaactcaccgcatcgggcgcccgcgtcaccatcgccgcctgcgacgtcgccgacccccacgccatgcgcaccctcctcgacgccatccccgccgagacgcccctcaccgccgtcgtccacaccgccggcgcaccgggcggcgatccgctggacgtcaccggcccggaggacatcgcccgcatcctgggcgcgaagacgagcggcgccgaggtcctcgacgacctgctccgcggcactccgctggacgccttcgtcctctactcctcgaacgccggggtctggggcagcggcagccagggcgtctacgcggcggccaacgcccacctcgacgcgctcgccgcccggcgccgcgcccggggcgagacggcgacctcggtcgcctggggcctctgggccggcgacggcatgggccggggcgccgacgacgcgtactggcagcgtcgcggcatccgtccgatgagccccgaccgcgccctggacgaactggccaaggccctgagccacgacgagaccttcgtcgccgtggccgatgtcgactgggagcggttcgcgcccgcgttcacggtgtcccgtcccagccttctgctcgacggcgtcccggaggcccggcaggcgctcgccgcacccgtcggtgccccggctcccggcgacgccgccgtggcgccgaccgggcagtcgtcggcgctggccgcgatcaccgcgctccccgagcccgagcgccggccggcgctcctcaccctcgtccgtacccacgcggcggccgtactcggccattcctcccccgaccgggtggcccccggccgtgccttcaccgagctcggcttcgactcgctgacggccgtgcagctccgcaaccagctctccacggtggtcggcaacaggctccccgccaccacggtcttcgaccacccgacgcccgccgcactcgccgcgcacctccacgaggcgtacctcgcaccggccgagccggccccgacggactgggaggggcgggtgcgccgggccctggccgaactgcccctcgaccggctgcgggacgcgggggtcctcgacaccgtcctgcgcctcaccggcatcgagcccgagccgggttccggcggttcggacggcggcgccgccgaccctggtgcggagccggaggcgtcgatcgacgacctggacgccgaggccctgatccggatggctctcggcccccgtaacacctga

SEQ ID NO: 35 - protein, 1562 aa, Streptomyces venezuelae
MANNEDKLRDYLKRVTAELQQNTRRLREIEGRTHEPVAIVGMACRLPGGVASPEDLWQLVAGDGDAISEFPQDRGWDVEGLYDPDPDASGRTYCRSGGFLHDAGEFDADFFGISPREALAMDPQQRLSLTTAWEAIESAGIDPTALKGSGLGVFVGGWHTGYTSGQTTAVQSPELEGHLVSGAALGFLSGRIAYVLGTDGPALTVDTACSSSLVALHLAVQALRKGECDMALAGGVTVMPNADLFVQFSRQRGLAADGRSKAFATSADGFGPAEGAGVLLVERLSDARRNGHRILAVVRGSAVNQDGASNGLTAPHGPSQQRVIRRALADARLAPGDVDVVEAHGTGTRLGDPIEAQALIATYGQEKSSEQPLRLGALKSNIGHTQAAAGVAGVIKMVQAMRHGLLPKTLHVDEPSDQIDWSAGTVELLTEAVDWPEKQDGGLRRAAVSSFGISGTNAHVVLEEAPAVEDSPAVEPPAGGGVVPWPVSAKTPAALDAQIGQLAAYADGRTDVDPAVAARALVDSRTAMEHRAVAVGDSREALRDALRMPEGLVRGTSSDVGRVAFVFPGQGTQWAGMGAELLDSSPEFAASMAECETALSRYVDWSLEAVVRQEPGAPTLDRVDVVQPVTFAVMVSLAKVWQHHGITPQAVVGHSQGEIAAAYVAGALTLDDAARVVTLRSKSIAAHLAGKGGMISLALDEAAVLKRLSDFDGLSVAAVNGPTATVVSGDPTQIEELARTCEADGVRARIIPVDYASHSRQVEIIEKELAEVLAGLAPQAPHVPFFSTLEGTWITEPVLDGTYWYRNLRHRVGFAPAVETLAVDGFTHFIEVSAHPVLTMTLPETVTGLGTLRREQGGQERLVTSLAEAWANGLTIDWAPILPTATGHHPELPTYAFQTERFWLQSSAPTSAADDWRYRVEWKPLTASGQADLSGRWIVAVGSEPEAELLGALKAAGAEVDVLEAGADDDREALAARLTALTTGDGFTGVVSLLDDLVPQVAWVQALGDAGIKAPLWSVTQGAVSVGRLDTPADPDRAMLWGLGRVVALEHPERWAGLVDLPAQPDAAALAHLVTALSGATGEDQIAIRTTGLHARRLARAPLHGRRPTRDWQPHGTVLITGGTGALGSHAARWMAHHGAEHLLLVSRSGEQAPGATQLTAELTASGARVTIAACDVADPHAMRTLLDAIPAETPLTAVVHTAGAPGGDPLDVTGPEDIARILGAKTSGAEVLDDLLRGTPLDAFVLYSSNAGVWGSGSQGVYAAANAHLDALAARRRARGETATSVAWGLWAGDGMGRGADDAYWQRRGIRPMSPDRALDELAKALSHDETFVAVADVDWERFAPAFTVSRPSLLLDGVPEARQALAAPVGAPAPGDAAVAPTGQSSALAAITALPEPERRPALLTLVRTHAAAVLGHSSPDRVAPGRAFTELGFDSLTAVQLRNQLSTVVGNRLPATTVFDHPTPAALAAHLHEAYLAPAEPAPTDWEGRVRRALAELPLDRLRDAGVLDTVLRLTGIEPEPGSGGSDGGAADPGAEPEASIDDLDAEALIRMALGPRNT

SEQ ID NO: 36 - DNA, 4041 bp, Streptomyces venezuelae
atgacgagttccaacgaacagttggtggacgctctgcgcgcctctctcaaggagaacgaagaactccggaaagagagccgtcgccgggccgaccgtcggcaggagcccatggcgatcgtcggcatgagctgccggttcgcgggcggaatccggtcccccgaggacctctgggacgccgtcgccgcgggcaaggacctggtctccgaggtaccggaggagcgcggctgggacatcgactccctctacgacccggtgcccgggcgcaagggcacgacgtacgtccgcaacgccgcgttcctcgacgacgccgccggattcgacgcggccttcttcgggatctcgccgcgcgaggccctcgccatggacccgcagcagcggcagctcctcgaagcctcctgggaggtcttcgagcgggccggcatcgaccccgcgtcggtccgcggcaccgacgtcggcgtgtacgtgggctgtggctaccaggactacgcgccggacatccgggtcgcccccgaaggcaccggcggttacgtcgtcaccggcaactcctccgccgtggcctccgggcgcatcgcgtactccctcggcctggagggacccgccgtgaccgtggacacggcgtgctcctcttcgctcgtcgccctgcacctcgccctgaagggcctgcggaacggcgactgctcgacggcactcgtgggcggcgtggccgtcctcgcgacgccgggcgcgttcatcgagttcagcagccagcaggccatggccgccgacggccggaccaagggcttcgcctcggcggcggacggcctcgcctggggcgagggcgtcgccgtactcctcctcgaacggctctccgacgcgcggcgcaagggccaccgggtcctggccgtcgtgcgcggcagcgccatcaaccaggacggcgcgagcaacggcctcacggctccgcacgggccctcccagcagcacctgatccgccaggccctggccgacgcgcggctcacgtcgagcgacgtggacgtcgtggagggccacggcacggggacccgtctcggcgacccgatcgaggcgcaggcgctgctcgccacgtacgggcaggggcgcgccccggggcagccgctgcggctggggacgctgaagtcgaacatcgggcacacgcaggccgcttcgggtgtcgccggtgtcatcaagatggtgcaggcgctgcgccacggggtgctgccgaagaccctgcacgtggacgagccgacggaccaggtcgactggtcggccggttcggtcgagctgctcaccgaggccgtggactggccggagcggccgggccggctccgccgggcgggcgtctccgcgttcggcgtgggcgggacgaacgcgcacgtcgtcctggaggaggccccggcggtcgaggagtcccctgccgtcgagccgccggccggtggcggcgtggtgccgtggccggtgtccgcgaagacctcggccgcactggacgcccagatcgggcagctcgccgcatacgcggaagaccgcacggacgtggatccggcggtggccgcccgcgccctggtcgacagccgtacggcgatggagcaccgcgcggtcgcggtcggcgacagccgggaggcactgcgggacgccctgcggatgccggaaggactggtacggggcacggtcaccgatccgggccgggtggcgttcgtcttccccggccagggcacgcagtgggccggcatgggcgccgaactcctcgacagctcacccgaattcgccgccgccatggccgaatgcgagaccgcactctccccgtacgtcgactggtctctcgaagccgtcgtccgacaggctcccagcgcaccgacactcgaccgcgtcgacgtcgtccagcccgtcaccttcgccgtcatggtctccctcgccaaggtctggcagcaccacggcatcacccccgaggccgtcatcggccactcccagggcgagatcgccgccgcgtacgtcgccggtgccctcaccctcgacgacgccgctcgtgtcgtgaccctccgcagcaagtccatcgccgcccacctcgccggcaagggcggcatgatctccctcgccctcagcgaggaagccacccggcagcgcatcgagaacctccacggactgtcgatcgccgccgtcaacgggcctaccgccaccgtggtttcgggcgaccccacccagatccaagaacttgctcaggcgtgtgaggccgacggcatccgcgcacggatcatccccgtcgactacgcctcccacagcgcccacgtcgagaccatcgagaacgaactcgccgacgtcctggcggggttgtccccccagacaccccaggtccccttcttctccaccctcgaaggcacctggatcaccgaacccgccctcgacggcggctactggtaccgcaacctccgccatcgtgtgggcttcgccccggccgtcgagaccctcgccaccgacgaaggcttcacccacttcatcgaggtcagcgcccaccccgtcctcaccatgaccctccccgacaaggtcaccggcctggccaccctccgacgcgaggacggcggacagcaccgcctcaccacctcccttgccgaggcctgggccaacggcctcgccctcgactgggcctccctcctgcccgccacgggcgccctcagccccgccgtccccgacctcccgacgtacgccttccagcaccgctcgtactggatcagccccgcgggtcccggcgaggcgcccgcgcacaccgcttccgggcgcgaggccgtcgccgagacggggctcgcgtggggcccgggtgccgaggacctcgacgaggagggccggcgcagcgccgtactcgcgatggtgatgcggcaggcggcctccgtgctccggtgcgactcgcccgaagaggtccccgtcgaccgcccgctgcgggagatcggcttcgactcgctgaccgccgtcgacttccgcaaccgcgtcaaccggctgaccggtctccagctgccgcccaccgtcgtgttccagcacccgacgcccgtcgcgctcgccgagcgcatcagcgacgagctggccgagcggaactgggccgtcgccgagccgtcggatcacgagcaggcggaggaggagaaggccgccgctccggcgggggcccgctccggggccgacaccggcgccggcgccgggatgttccgcgccctgttccggcaggccgtggaggacgaccggtacggcgagttcctcgacgtcctcgccgaagcctccgcgttccgcccgcagttcgcctcgcccgaggcctgctcggagcggctcgacccggtgctgctcgccggcggtccgacggaccgggcggaaggccgtgccgttctcgtcggctgcaccggcaccgcggcgaacggcggcccgcacgagttcctgcggctcagcacctccttccaggaggagcgggacttcctcgccgtacctctccccggctacggcacgggtacgggcaccggcacggccctcctcccggccgatctcgacaccgcgctcgacgcccaggcccgggcgatcctccgggccgccggggacgccccggtcgtcctgctcgggcactccggcggcgccctgctcgcgcacgagctggccttccgcctggagcgggcgcacggcgcgccgccggccgggatcgtcctggtcgacccctatccgccgggccatcaggagcccatcgaggtgtggagcaggcagctgggcgagggcctgttcgcgggcgagctggagccgatgtccgatgcgcggctgctggccatgggccggtacgcgcggttcctcgccggcccgcggccgggccgcagcagcgcgcccgtgcttctggtccgtgcctccgaaccgctgggcgactggcaggaggagcggggcgactggcgtgcccactgggaccttccgcacaccgtcgcggacgtgccgggcgaccacttcacgatgatgcgggaccacgcgccggccgtcgccgaggccgtcctctcctggctcgacgccatcgagggcatcgagggggcgggcaagtga

SEQ ID NO: 37 - protein, 1346 aa, Streptomyces venezuelae
MTSSNEQLVDALRASLKENEELRKESRRRADRRQEPMAIVGMSCRFAGGIRSPEDLWDAVAAGKDLVSEVPEERGWDIDSLYDPVPGRKGTTYVRNAAFLDDAAGFDAAFFGISPREALAMDPQQRQLLEASWEVFERAGIDPASVRGTDVGVYVGCGYQDYAPDIRVAPEGTGGYVVTGNSSAVASGRIAYSLGLEGPAVTVDTACSSSLVALHLALKGLRNGDCSTALVGGVAVLATPGAFIEFSSQQAMAADGRTKGFASAADGLAWGEGVAVLLLERLSDARRKGHRVLAVVRGSAINQDGASNGLTAPHGPSQQHLIRQALADARLTSSDVDVVEGHGTGTRLGDPIEAQALLATYGQGRAPGQPLRLGTLKSNIGHTQAASGVAGVIKMVQALRHGVLPKTLHVDEPTDQVDWSAGSVELLTEAVDWPERPGRLRRAGVSAFGVGGTNAHVVLEEAPAVEESPAVEPPAGGGVVPWPVSAKTSAALDAQIGQLAAYAEDRTDVDPAVAARALVDSRTAMEHRAVAVGDSREALRDALRMPEGLVRGTVTDPGRVAFVFPGQGTQWAGMGAELLDSSPEFAAAMAECETALSPYVDWSLEAVVRQAPSAPTLDRVDVVQPVTFAVMVSLAKVWQHHGITPEAVIGHSQGEIAAAYVAGALTLDDAARVVTLRSKSIAAHLAGKGGMISLALSEEATRQRIENLHGLSIAAVNGPTATVVSGDPTQIQELAQACEADGIRARIIPVDYASHSAHVETIENELADVLAGLSPQTPQVPFFSTLEGTWITEPALDGGYWYRNLRHRVGFAPAVETLATDEGFTHFIEVSAHPVLTMTLPDKVTGLATLRREDGGQHRLTTSLAEAWANGLALDWASLLPATGALSPAVPDLPTYAFQHRSYWISPAGPGEAPAHTASGREAVAETGLAWGPGAEDLDEEGRRSAVLAMVMRQAASVLRCDSPEEVPVDRPLREIGFDSLTAVDFRNRVNRLTGLQLPPTVVFQHPTPVALAERISDELAERNWAVAEPSDHEQAEEEKAAAPAGARSGADTGAGAGMFRALFRQAVEDDRYGEFLDVLAEASAFRPQFASPEACSERLDPVLLAGGPTDRAEGRAVLVGCTGTAANGGPHEFLRLSTSFQEERDFLAVPLPGYGTGTGTGTALLPADLDTALDAQARAILRAAGDAPVVLLGHSGGALLAHELAFRLERAHGAPPAGIVLVDPYPPGHQEPIEVWSRQLGEGLFAGELEPMSDARLLAMGRYARFLAGPRPGRSSAPVLLVRASEPLGDWQEERGDWRAHWDLPHTVADVPGDHFTMMRDHAPAVAEAVLSWLDAIEGIEGAGK

SEQ ID NO: 38 - DNA, 1251 bp, Streptomyces venezuelae
gtgcgccgtacccagcagggaacgaccgcttctcccccggtactcgacctcggggccctggggcaggatttcgcggccgatccgtatccgacgtacgcgagactgcgtgccgagggtccggcccaccgggtgcgcacccccgagggggacgaggtgtggctggtcgtcggctacgaccgggcgcgggcggtcctcgccgatccccggttcagcaaggactggcgcaactccacgactcccctgaccgaggccgaggccgcgctcaaccacaacatgctggagtccgacccgccgcggcacacccggctgcgcaagctggtggcccgtgagttcaccatgcgccgggtcgagttgctgcggccccgggtccaggagatcgtcgacgggctcgtggacgccatgctggcggcgcccgacggccgcgccgatctgatggagtccctggcctggccgctgccgatcaccgtgatctccgaactcctcggcgtgcccgagccggaccgcgccgccttccgcgtctggaccgacgccttcgtcttcccggacgatcccgcccaggcccagaccgccatggccgagatgagcggctatctctcccggctcatcgactccaagcgcgggcaggacggcgaggacctgctcagcgcgctcgtgcggaccagcgacgaggacggctcccggctgacctccgaggagctgctcggtatggcccacatcctgctcgtcgcggggcacgagaccacggtcaatctgatcgccaacggcatgtacgcgctgctctcgcaccccgaccagctggccgccctgcgggccgacatgacgctcttggacggcgcggtggaggagatgttgcgctacgagggcccggtggaatccgcgacctaccgcttcccggtcgagcccgtcgacctggacggcacggtcatcccggccggtgacacggtcctcgtcgtcctggccgacgcccaccgcacccccgagcgcttcccggacccgcaccgcttcgacatccgccgggacaccgccggccatctcgccttcggccacggcatccacttctgcatcggcgcccccttggcccggttggaggcccggatcgccgtccgcgcccttctcgaacgctgcccggacctcgccctggacgtctcccccggcgaactcgtgtggtatccgaacccgatgattcgcgggctcaaggccctgccgatccgctggcggcgaggacgggaggcgggccgccgtaccggttga

SEQ ID NO: 39 - protein, 416 aa, Streptomyces venezuelae
MRRTQQGTTASPPVLDLGALGQDFAADPYPTYARLRAEGPAHRVRTPEGDEVWLVVGYDRARAVLADPRFSKDWRNSTTPLTEAEAALNHNMLESDPPRHTRLRKLVAREFTMRRVELLRPRVQEIVDGLVDAMLAAPDGRADLMESLAWPLPITVISELLGVPEPDRAAFRVWTDAFVFPDDPAQAQTAMAEMSGYLSRLIDSKRGQDGEDLLSALVRTSDEDGSRLTSEELLGMAHILLVAGHETTVNLIANGMYALLSHPDQLAALRADMTLLDGAVEEMLRYEGPVESATYRFPVEPVDLDGTVIPAGDTVLVVLADAHRTPERFPDPHRFDIRRDTAGHLAFGHGIHFCIGAPLARLEARIAVRALLERCPDLALDVSPGELVWYPNPMIRGLKALPIRWRRGREAGRRTG

SEQ ID NO: 40 - DNA, 2787 bp, Streptomyces venezuelae
atgaatctggtggaacgcgacggggagatagcccatctcagggccgttcttgacgcatccgccgcaggtgacgggacgctcttactcgtctccggaccggccggcagcgggaagacggagctgctgcggtcgctccgccggctggccgccgagcgggagacccccgtctggtcggtccgggcgctgccgggtgaccgcgacatccccctgggcgtcctctgccagttactccgcagcgccgaacaacacggtgccgacacctccgccgtccgcgacctgctggacgccgcctcgcggcgggccggaaacctcacctcccccgccgacgcgccgctccgcgtcgacgagacacaccgcctgcacgactggctgctctccgtctcccgccgcaccccgttcctcgtcgccgtcgacgacctgacccacgccgacaccgcgtccctgaggttcctcctgtactgcgccgcccaccacgaccagggcggcatcggcttcgtcatgaccgagcgggcctcgcagcgcgccggataccgggtgttccgcgccgagctgctccgccagccgcactgccgcaacatgtggctctccgggcttccccccagcggggtacgccagttactcgcccactactacggccccgaggccgccgagcggcgggcccccgcgtaccacgcgacgaccggcgggaacccgctgctcctgcgggcgctgacccaggaccggcaggcctcccacaccaccctcggcgcggccggcggcgacgagcccgtccacggcgacgccttcgcccaggccgtcctcgactgcctgcaccgcagcgccgagggcacactggagaccgcccgctggctcgcggtcctcgaacagtccgacccgctcctggtggagcggctcacgggaacgaccgccgccgccgtcgagcgccacatccaggagctcgccgccatcggcctcctggacgaggacggcaccctgggacagcccgcgatccgcgaggccgccctccaggacctgccggccggcgagcgcaccgaactgcaccggcgcgccgcggagcagctgcaccgggacggcgccgacgaggacaccgtggcccgccacctgctggtcggcggcgcccccgacgctccctgggcgctgcccctgctcgaacggggcgcgcagcaggccctgttcgacgaccgactcgacgacgccttccggatcctcgagttcgccgtgcggtcgagcaccgacaacacccagctggcccgcctcgccccacacctggtcgcggcctcctggcggatgaacccgcacatgacgacccgggccctcgcactcttcgaccggctcctgagcggtgaactgccgcccagccacccggtcatggccctgatccgctgcctcgtctggtacgggcggctgcccgaggccgccgacgcgctgtcccggctgcggcccagctccgacaacgatgccttggagctgtcgctcacccggatgtggctcgcggcgctgtgcccgccgctcctggagtccctgccggccacgccggagccggagcggggtcccgtccccgtacggctcgcgccgcggacgaccgcgctccaggcccaggccggcgtcttccagcggggcccggacaacgcctcggtcgcgcaggccgaacagatcctgcagggctgccggctgtcggaggagacgtacgaggccctggagacggccctcttggtcctcgtccacgccgaccggctcgaccgggcgctgttctggtcggacgccctgctcgccgaggccgtggagcggcggtcgctcggctgggaggcggtcttcgccgcgacccgggcgatgatcgcgatccgctgcggcgacctcccgacggcgcgggagcgggccgagctggcgctctcccacgcggcgccggagagctggggcctcgccgtgggcatgcccctctccgcgctgctgctcgcctgcacggaggccggcgagtacgaacaggcggagcgggtcctgcggcagccggtgccggacgcgatgttcgactcgcggcacggcatggagtacatgcacgcccggggccgctactggctggcgacgggccggctgcacgcggcgctgggcgagttcatgctctgcggggagatcctgggcagctggaacctcgaccagccctcgatcgtgccctggcggacctccgccgccgaggtgtacctgcggctcggcaaccgccagaaggccagggcgctggccgaggcccagctcgccctggtgcggcccgggcgctcccgcacccggggtctcaccctgcgggtcctggcggcggcggtggacggccagcaggcggagcggctgcacgccgaggcggtcgacatgctgcacgacagcggcgaccggctcgaacacgcccgcgcgctcgccgggatgagccgccaccagcaggcccagggggacaactaccgggcgaggatgacggcgcggctcgccggcgacatggcgtgggcctgcggcgcgtacccgctggccgaggagatcgtgccgggccgcggcggccgccgggcgaaggcggtgagcacggagctggaactgccgggcggcccggacgtcggcctgctctcggaggccgaacgccgggtggcggccctggcagcccgaggattgacgaaccgccagatagcgcgccggctctgcgtcaccgcgagcacggtcgaacagcacctgacgcgcgtctaccgcaaactgaacgtgacccgccgagcagacctcccgatcagcctcgcccaggacaagtccgtcacggcctga

SEQ ID NO: 41 - protein, 928 aa, Streptomyces venezuelae
MNLVERDGEIAHLRAVLDASAAGDGTLLLVSGPAGSGKTELLRSLRRLAAERETPVWSVRALPGDRDIPLGVLCQLLRSAEQHGADTSAVRDLLDAASRRAGNLTSPADAPLRVDETHRLHDWLLSVSRRTPFLVAVDDLTHADTASLRFLLYCAAHHDQGGIGFVMTERASQRAGYRVFRAELLRQPHCRNMWLSGLPPSGVRQLLAHYYGPEAAERRAPAYHATTGGNPLLLRALTQDRQASHTTLGAAGGDEPVHGDAFAQAVLDCLHRSAEGTLETARWLAVLEQSDPLLVERLTGTTAAAVERHIQELAAIGLLDEDGTLGQPAIREAALQDLPAGERTELHRRAAEQLHRDGADEDTVARHLLVGGAPDAPWALPLLERGAQQALFDDRLDDAFRILEFAVRSSTDNTQLARLAPHLVAASWRMNPHMTTRALALFDRLLSGELPPSHPVMALIRCLVWYGRLPEAADALSRLRPSSDNDALELSLTRMWLAALCPPLLESLPATPEPERGPVPVRLAPRTTALQAQAGVFQRGPDNASVAQAEQILQGCRLSEETYEALETALLVLVHADRLDRALFWSDALLAEAVERRSLGWEAVFAATRAMIAIRCGDLPTARERAELALSHAAPESWGLAVGMPLSALLLACTEAGEYEQAERVLRQPVPDAMFDSRHGMEYMHARGRYWLATGRLHAALGEFMLCGEILGSWNLDQPSIVPWRTSAAEVYLRLGNRQKARALAEAQLALVRPGRSRTRGLTLRVLAAAVDGQQAERLHAEAVDMLHDSGDRLEHARALAGMSRHQQAQGDNYRARMTARLAGDMAWACGAYPLAEEIVPGRGGRRAKAVSTELELPGGPDVGLLSEAERRVAALAARGLTNRQIARRLCVTASTVEQHLTRVYRKLNVTRRADLPISLAQDKSVTA

SEQ ID NO: 42 - DNA, 27 bp, Streptomyces venezuelae
cccgaattcgccgccgccatggccgaa

SEQ ID NO: 43 - DNA, 35 bp, Streptomyces venezuelae
gtgatgcatcggctcggcgacggcccagttccgct

SEQ ID NO: 44 - DNA, 45 bp, Streptomyces venezuelae
atgcatcaccaccaccaccactgagggggcgggcaagtgaccgac

SEQ ID NO: 45 - DNA, 33 bp, Streptomyces venezuelae
gggtctagagctgcaccggcgggtcgtagcgga

SEQ ID NO: 46 - DNA, 27 bp, Streptomyces venezuelae
gaattcatcgagggggcgggcaagtga

SEQ ID NO: 47 - DNA, 31 bp, Streptomyces venezuelae
atgcatcaggtcgtcggtcaccgtgggttct

SEQ ID NO: 48 - DNA, 30 bp, Streptomyces venezuelae
ggatccgcgccgggatgttccgcgccctgt

SEQ ID NO: 49 - DNA, 31 bp, Streptomyces venezuelae
aaaatgcatcagaggtctgtcggtcacttgc

SEQ ID NO: 50 - DNA, 41 bp, Streptomyces venezuelae
aaaagatcttgatggtgcaggcgctgcgccacggggtgctg

SEQ ID NO: 51 - DNA, 32 bp, Streptomyces venezuelae
aaaagatctccaacgaacagttggtggacgct

SEQ ID NO: 52 - DNA, 846 bp, Streptomyces venezuelae
gtgaccgacagacctctgaacgtggacagcggactgtggatccggcgcttccaccccgcgccgaacagcgcggtgcggctggtctgcctgccgcacgccggcggctccgccagctacttcttccgcttctcggaggagctgcacccctccgtcgaggccctgtcggtgcagtatccgggccgccaggaccggcgtgccgagccgtgtctggagagcgtcgaggagctcgccgagcatgtggtcgcggccaccgaaccctggtggcaggagggccggctggccttcttcgggcacagcctcggcgcctccgtcgccttcgagacggcccgcatcctggaacagcggcacggggtacggcccgagggcctgtacgtctccggtcggcgcgccccgtcgctggcgccggaccggctcgtccaccagctggacgaccgggcgttcctggccgagatccggcggctcagcggcaccgacgagcggttcctccaggacgacgagctgctgcggctggtgctgcccgcgctgcgcagcgactacaaggcggcggagacgtacctgcaccggccgtccgccaagctcacctgcccggtgatggccctggccggcgaccgtgacccgaaggcgccgctgaacgaggtggccgagtggcgtcggcacaccagcgggccgttctgcctccgggcgtactccggcggccacttctacctcaacgaccagtggcacgagatctgcaacgacatctccgaccacctgctcgtcacccgcggcgcgcccgatgcccgcgtcgtgcagcccccgaccagccttatcgaaggagcggcgaagagatggcagaacccacggtga

SEQ ID NO: 53 - protein, 281 aa, Streptomyces venezuelae
MTDRPLNVDSGLWIRRFHPAPNSAVRLVCLPHAGGSASYFFRFSEELHPSVEALSVQYPGRQDRRAEPCLESVEELAEHVVAATEPWWQEGRLAFFGHSLGASVAFETARILEQRHGVRPEGLYVSGRRAPSLAPDRLVHQLDDRAFLAEIRRLSGTDERFLQDDELLRLVLPALRSDYKAAETYLHRPSAKLTCPVMALAGDRDPKAPLNEVAEWRRHTSGPFCLRAYSGGHFYLNDQWHEICNDISDHLLVTRGAPDARVVQPPTSLIEGAAKRWQNPR